(19) United States
(12) Patent Application Publication
(10) Pub. No.: US 2007/0249670 A1
(43) Pub. Date: Oct. 25, 2007

(54) Glycogen Phosphorylase Inhibitor Compounds and Pharmaceutical Compositions Thereof

(75) Inventors: Karen Evans, Collegeville, PA (US); Maria Cichy-Knight, Collegeville, PA (US); Frank Teen Coppo, Collegeville, PA (US); Kate Ann Dwornik, Durham, NC (US); Jennifer Paul Gale, Durham, NC (US); Dulce Maria Garrido, Durham, NC (US); Yue Hu Li, Collegeville, PA (US); Mehul P. Patel, Collegeville, PA (US); Francis X. Tavares, Durham, NC (US); Stephen Andrew Thomson, Durham, NC (US); Scott Howard Dickerson, Durham, NC (US); Andrew James Peat, Durham, NC (US); Steven Meagher Sparks, Durham, NC (US); Pierette Banker, Durham, NC (US); Joel P. Cooper, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 11/718,804

(22) PCT Filed: Nov. 4, 2005
(86) PCT No.: PCT/US05/39956

§ 371 Date: May 8, 2007

Related U.S. Application Data
(60) Provisional application No. 60/626,389, filed on Nov. 9, 2004.

(51) Int. Cl. A61K 31/24; A61K 31/381; A61K 31/40; A61K 31/4164; A61K 31/445; A61K 31/47; C07C 229/72; C07D 207/16; C07D 211/18; C07D 215/38; C07D 233/64; C07D 333/50

(52) U.S. Cl. 514/313; 514/319; 514/400; 514/423; 514/438; 514/443; 514/539; 546/162; 546/205; 548/334.1; 548/533; 549/57; 560/34

(57) ABSTRACT

The invention relates to glycogen phosphorylase inhibitor compounds, pharmaceutical compositions of these compounds, methods of treatment using the pharmaceutical compositions to treat diabetes, conditions associated with diabetes, and/or tissue ischemia, including myocardial ischemia, and processes for making the compounds.

### FIELD OF THE INVENTION

The present invention relates to glycogen phosphorylase inhibitor compounds, pharmaceutical compositions of these compounds, the use of these compounds or pharmaceutical compositions containing them in the treatment of diabetes, conditions associated with diabetes, and/or tissue ischemia including myocardial ischemia, and processes for making the compounds.

### BACKGROUND OF THE INVENTION

Treatment of diabetes remains less than satisfactory. In addition, recently compiled data from the World Health Organization (WHO) show that approximately 150 million people have diabetes mellitus worldwide, and that this number may well double by the year 2025.

A number of drugs are available for the treatment of diabetes. These include injected insulin and drugs such as sulfonylureas, glipizide, tobutamide, acetohexamide, tolazimide, biguanides, and metformin (glucophage) which are ingested orally. Insulin self-injection is required in diabetic patients in which orally ingested drugs are not effective. Patients having Type 1 diabetes (also referred to as insulin dependent diabetes mellitus) are usually treated by self-injecting insulin. Patients suffering from Type 2 diabetes (also referred to as non-insulin dependent diabetes mellitus) are usually treated with a combination of diet, exercise, and an oral agent. When oral agents fail insulin may be prescribed. When diabetic drugs are taken orally usually multiple daily doses are often required.

Determination of the proper dosage of insulin requires frequent testing of the sugar in urine and/or blood. The administration of an excess dose of insulin generally causes hypoglycemia which has symptoms ranging from mild abnormalities in blood glucose to coma, or even death. Orally ingested drugs are likewise not without undesirable side effects. For example, such drugs can be ineffective in some patients and cause gastrointestinal disturbances or impair proper liver function in other individuals. There is a continuing need for drugs having fewer side effects and/or ones that succeed where others fail.

In Type 2 or non-insulin dependent diabetes mellitus, hepatic glucose production is an important target. The liver is the major regulator of plasma glucose levels in the fasting state. The rate of hepatic glucose production in Type 2 patients is typically significantly elevated when compared to normal (non-diabetic) individuals. For Type 2 diabetics, in the fed or postprandial state, the liver has a proportionately smaller role in the total plasma glucose supply, and hepatic glucose production is abnormally high.

The liver produces glucose by glycogenolysis (breakdown of the glucose polymer glycogen) and gluconeogenesis (synthesis of glucose from 2- and 3-carbon precursors). Glycogenolysis therefore is an important target for interruption of hepatic glucose production. There is some evidence to suggest that glycogenolysis may contribute to the inappropriate hepatic glucose output in Type 2 diabetic patients. Individuals having liver glycogen storage diseases such as Hers' disease or glycogen phosphorylase deficiency often display episodic hypoglycemia. Further, in normal post-absorptive humans up to about 75% of hepatic glucose production is estimated to result from glycogenolysis.

Glycogenolysis is catalyzed in liver, muscle, and brain by tissue-specific isoforms of the enzyme glycogen phosphorylase. This enzyme cleaves the glycogen macromolecule to release glucose-1-phosphate and a shortened glycogen macromolecule.

Glycogen phosphorylase inhibitors include glucose and its analogs, caffeine and other purine analogs, cyclic amines with various substitutents, and indole-like compounds. These compounds and glycogen phosphorylase inhibitors in general have been postulated to be of potential use in the treatment of Type 2 diabetes by decreasing hepatic glucose production and lowering lycemia. Furthermore, we believe it maybe desirable that a glycogen phosphorylase inhibitor be sensitive to glucose concentrations in blood. Several different types of glycogen phoshorylase inhibitors have been reported. These include glucose and glucose analogs, caffeine and other purine analogs, indole compounds and acyl ureas.

Accordingly, what is desired is a new compound and pharmaceutical composition thereof for the treatment of diabetes and/or conditions associated with diabetes.

### SUMMARY OF THE INVENTION

The present invention provides a novel glycogen phosphorylase inhibitor compound and a pharmaceutical composition thereof that binds to at least one site of the glycogen phosphorylase enzyme. We believe that this novel glycogen phosphorylase inhibitor compound and a pharmaceutical composition thereof bind to the AMP allosteric binding site, and are glucose sensitive.

In one embodiment of the invention there is provided a novel compound of Formula 1 comprising:

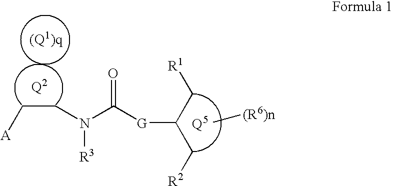

a pharmaceutically acceptable salt, solvate, or physiologically functional derivative thereof

wherein:

A is C(═O)NQ³Q⁴ or C(═O)OH;

Q¹ and Q² are fused together;

Q¹ is selected from the group consisting of (i) a 5- or 6-membered aromatic ring, (ii) a 5- or 6-membered cycloalkyl ring, (iii) a 5- or 6-membered heteroaromatic ring having at least one heteroatom selected from the group consisting of nitrogen, oxygen, or sulfur, and (iv) a 4- to 8-membered heterocyclic ring having at least one heteroatom selected from the group consisting of nitrogen, oxygen, or sulfur; and q is 0 or 1;

Q² is selected from the group consisting of (i) a 5- or 6-membered aromatic ring and (ii) a 5- or 6-membered heteroaromatic ring having at least one heteroatom selected from the group consisting of nitrogen, oxygen, or sulfur;

R¹ and R² are each independently selected from the group consisting of hydrogen, C₁₋₆ alkyl, halo (Cl, Br, I, and F), alkoxy, monoalkylamino, and dialkylamino;

R³ is hydrogen or a C₁₋₆ alkyl;

Q³ and Q⁴ are each independently selected from the group consisting of (i) hydrogen, (ii) C₁₋₆ alkyl, (iii) —CR⁴R⁵Z, where Z is a 5- or 6-membered heteroaryl having at least one heteroatom selected from the group consisting of nitrogen, oxygen, and sulfur, (iv) aryl, and (v) —CR⁴R⁵COOH;

R⁴ and R⁵ are each independently selected from the group consisting of (i) hydrogen, (ii) a C₁₋₆ alkyl, (iii) a 4- to 8-membered cycloalkyl, (iv) a 5- or 6-membered aryl, (v) a 5- or 6-membered heteroaryl, (vi) a 5- or 6-membered aralkyl, (vii) a 5- or 6-membered heteroaralkyl, having at least one heteroatom selected from the group consisting of nitrogen, oxygen and sulfur, (viii) a 4- to 8-membered cycloalkylalkyl, and (ix) a 4- to 8-membered heterocyclic ring;

R⁴ and R⁵ taken together can form a (i) 3-10 membered cycloalkyl or (ii) a 4-8 membered heterocyclic ring;

G is selected from the group consisting of carbon, nitrogen, oxygen, and sulfur;

Q⁵ is selected from the group consisting of (i) a 5- or 6-membered aromatic ring and (ii) a 5- or 6-membered heteroaromatic ring having at least one heteroatom selected from the group consisting of nitrogen, oxygen, and sulfur; and

R⁶ is selected from the group consisting of (i) C₁₋₆ alkyl, (ii) halogen, (iii) alkoxy, (iv) cyano, (v) hydroxyl, (vi) haloalkyl, (vii) mono- or dialkyl-amino, (viii) 3-5 membered cycloalkyl, (ix) 3-5 membered cycloalkylalkyl, (x) alkenyl, (xi) alkyny, and (xii) acyl; and n is 0 or 1.

Another embodiment of the invention provides a pharmaceutical composition comprising the above-identified compound of Formula 1, a pharmaceutically acceptable salt, solvate, or physiologically functional derivative thereof and at least one excipient.

In one aspect of the invention there is provided a method of treating a mammal, especially a human, suffering from diabetes, a condition associated with diabetes or both comprising the administration, preferably orally, of a compound of Formula 1, a pharmaceutically acceptable salt, solvate, or physiologically functional derivative thereof. There is further provided a method of treating a mammal, especially a human, suffering from diabetes, a condition associated with diabetes or both comprising the administration, preferably orally, of a pharmaceutical composition comprising a compound of Formula 1, a pharmaceutically acceptable salt, solvate, or physiologically functional derivative thereof to the mammal.

There is provided a method of treating a mammal, especially a human suffering from tissue ischemia, particularly myocardial ischemia, comprising administering to said mammal a compound of Formula 1, a pharmaceutically acceptable salt, solvate, or physiologically functional derivative thereof. There is further provided a method of treating a mammal, especially a human, suffering from tissue ischemia, particularly myocardial ischemia, comprising the administration of a pharmaceutical composition containing a compound of Formula 1, a pharmaceutically acceptable salt, solvate, or physiological functional derivative thereof to said mammal.

In another aspect of the invention, there is provided a process for preparing a compound of Formula 1.

In yet another aspect of the invention, the invention provides the use of a compound of Formula 1 or a pharmaceutically acceptable salt, solvate, or physiologically functional derivative thereof for the preparation or manufacture of a medicine for treating diabetes and/or a condition associated with diabetes in a mammal, including a human.

In still another aspect of the invention, the invention provides the use of a pharmaceutical composition of the compound of Formula I or a pharmaceutically acceptable salt, solvate, or physiologically functional derivative thereof for the preparation or manufacture of a medicine, such as a medicine for treating diabetes and/or a condition associated with diabetes in a mammal, including a human.

### DETAILED DESCRIPTION OF THE INVENTION

As used herein, “a compound of the invention” or “a compound of Formula 1” means a compound of Formula I or a pharmaceutically acceptable salt, solvate, or physiologically functional derivative thereof. Similarly, with respect to isolatable intermediates the phrase means a compound having the formula and pharmaceutically acceptable salts, solvates, and physiologically functional derivatives thereof.

As used herein, the terms “alkyl” (and “alkylene”) refer to straight or branched hydrocarbon chains containing from 1 to 8 carbon atoms. Examples of “alkyl” as used herein include, but are not limited to, methyl, ethyl, n-propyl, n-butyl, n-pentyl, isobutyl, isopropyl, and tert-butyl. Examples of “alkylene” as used herein include, but are not limited to, methylene, ethylene, propylene, butylenes, and isobutylene. “Alkyl” also includes substituted alkyl. The alkyl groups may be optionally substituted one or more times with hydroxyl, alkyl, alkoxy, halo, amino, thio, carboxyl, amido, guanidino, and cyano.

As used herein, the term “cycloalkyl” refers to a non-aromatic carbocyclic ring having from 3 to 8 carbon atoms (unless otherwise specified) and no carbon-carbon double bonds. “Cycloalkyl” includes by way of example cyclopropyl, cyclobutyl, cyclopentyl, cyclohexyl, cycloheptyl, and cyclooctyl. “Cycloalkyl” also includes substituted cycloalkyl. The cycloalkyl may be optionally substituted with substituents selected from the group consisting of hydroxyl, cyano, halo, alkoxy, and alkyl.

The term “cycloalkylalkyl” refers to a cycloalkyl group as defined hereinbefore attached to an alkyl group, for example, cyclopropylmethyl, cyclohexylethyl, and the like.

As used herein, unless otherwise specified, the term “alkenyl” refers to straight or branched hydrocarbon chains containing 2 to 8 carbon atoms and at least one and up to three carbon-carbon double bonds. Examples of “alkenyl” as used herein include, but are not limited to, ethenyl and propenyl. “Alkenyl” also includes substituted alkenyl. The alkenyl groups may be optionally substituted with alkyl, halo, hydroxyl, alkoxy, and cyano.

As used herein, unless otherwise specified, the term “alkynyl” refers to straight or branched hydrocarbon chains containing 2 to 8 carbon atoms and at least on and up to three carbon-carbon triple bonds. Examples of “alkynyl as used herein include, but are not limited to, ethynyl, propynyl and butynyl.

As used herein, unless otherwise specified, the term “cycloalkenyl” refers to a non-aromatic carbocyclic ring having 3 to 8 carbon atoms (unless otherwise specified) and up to 3 carbon-carbon double bonds. “Cycloalkenyl” includes, by way of example, cyclobutenyl, cyclopentenyl, and cyclohexenyl. “Cycloalkenyl” also includes substituted cycloalkenyl. The ring may be optionally substituted with at lease one substituent selected from the group consisting of cyano, halo, hydroxyl, NH₂, —N₃, —CN, —O—C₁₋₃alkyl, —NH(C₁₋₃alkyl), —N(C₁₋₃alkyl)₂, and C₁₋₃alkyl (including haloalkyl).

As used herein, the terms “halo” or “halogen” refer to fluorine, chlorine, bromine, and iodine. Preferred among these are chlorine (or chloro) and fluorine (or fluoro).

The term “alkoxy” includes both branched and straight chain alkyl groups attached to a terminal oxygen atom. Typical alkoxy groups include methoxy, ethoxy, n-propoxy, isopropoxy, tert-butoxy, trifluoromethoxy, and the like.

The term “monoalkylamino” refers to an alkyl group attached to a nitrogen atom, for example, methylamino, isopropylamino, and the like.

The term “dialkylamino” refers to two alkyl groups, which may be the same or different, attached to a nitrogen atom, for example, dimethylamino, N-ethyl-N-methylamino, and the like.

As used herein, unless otherwise specified, the term “aryl” refers to monocyclic carbocyclic groups and fused bicyclic carbocyclic groups having from 6 to 12 carbon atoms and having at least one aromatic ring. Examples of particular aryl groups include, but are not limited to, phenyl and naphthyl. “Aryl” also includes substituted aryl, especially substituted phenyl. Aryl rings may be optionally substituted with substituents selected from the group consisting of halo, alkyl (including haloalkyl), alkenyl, cycloalkyl, cycloalkenyl, alkoxy, amino, hydroxy, hydroxyalkyl, aminoalkyl, carboxy, carboxamide, sulfonamide, aryl, heteroaryl (abbreviated as “Het”), amidine, cyano, nitro, and azido. Preferred aryl groups include, but are not limited to, phenyl, substituted phenyl, substituted thienyl, and substituted pyridyl. Preferred substituted phenyl is a phenyl containing one or more halo groups, particularly chloro and fluoro groups. Preferred substituted thienyl is a thienyl containing one or more alkyl groups, particularly methyl. Preferred substituted pyridyl is a pyridyl containing one or more alkyl groups, particularly methyl.

The term “aralkyl” is used to describe a group wherein the alkyl chain can be branched or straight chain with the aryl portion, as defined hereinbefore, forming a terminal portion of the aralkyl moiety. Examples of aralkyl groups include, but are not limited to, optionally substituted benzyl and phenethyl such as 4-chlorobenzyl, 2,4-dibromobenzyl, 2-methylbenzyl, 2-(3-fluorophenyl)ethyl, 2-(4-methylphenyl)ethyl, 2-(4-trifluoromethyl)phenyl)ethyl, 2-(2-methoxyphenyl)ethyl, 2-(3,5-dimethoxyphenyl)ethyl, 4-(trifluoromethoxy)benzyl, 4-hydroxybenzyl, and the like.

As used herein, the term “heterocyclic,” unless otherwise specified, refers to monocyclic saturated or unsaturated non-aromatic groups and fused bicyclic non-aromatic groups, having the specified number of members (e.g., carbon and heteroatoms N and/or O and/or S) in a single ring and containing 1, 2, 3, or 4 heteroatoms selected from N, O, and S. Examples of particular heterocyclic groups include, but are not limited to, tetrahydrofuran, dihydropyran, tetrahydropyran, pyran, oxetane, thietane, 1,4-dioxane, 1,3-dioxane, 1,3-dioxalane, piperidine, piperazine, tetrahydropyrimidine, pyrrolidine, morpholine, thiomorpholine, thiazolidine, oxazolidine, tetrahydrothiopyran, hydrotiophene, and the like. “Heterocyclic” also includes substituted heterocyclic. The heterocyclic group may be optionally substituted with substituents selected from the group consisting of halo, alkyl (including haloalkyls), alkenyl, cycloakyl, cycloalkenyl, perfluoroalkyl, alkoxy, amino, hydroxyl, alkylhydroxy, alkylamine, carboxy, carboxamide, sulfonamide, heteroaryl, amidine, cyano, nitro, and azido. Preferred heterocyclic groups according to the invention include, but are not limited to, piperidine and tetrahydropyran.

As used herein, the term “heteroaryl,” unless otherwise specified refers to aromatic monocyclic groups and aromatic fused bicyclic groups having the specified number of members (e.g., carbon and heteroatoms N and/or O and/or S) and containing 1, 2, 3, or 4 heteroatoms selected from N, O, and S. Examples of particular heteroaryl groups include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, and indazole. “Heteroaryl” also includes substituted heteroaryl. The heteroaryl group may be optionally substituted with substituents selected from the group consisting of halo, alkyl (including perhalo alkyl, e.g., perfluoroalkyl), aryl, alkenyl, cycloalkyl, cycloalkenyl, alkoxy, amino, hydroxy, alkylhydroxy, alkylamine, carboxy, carboxamide, sulfonamide, heteroaryl, amidine, cyano, nitro, and azido. Preferred heteroaryl groups according to the invention include, but are not limited to substituted and unsubstituted pyridine, thiophene, thiazole, imidazole, isoxazole, and indole.

The term “heteroaralkyl” is used to describe a group wherein the alkyl chain can be branched or straight chain with the heteroaryl portion, as defined hereinbefore, forming a terminal portion of the heteroalkyl moiety, for example, 3-furylmethyl, thenyl (thienylmethyl), furfuryl, indolyl, imidazolyl, and the like.

A used herein, the term “optionally” means that the subsequently described event(s) may or may not occur, and includes both event(s) that occur and event(s) that do not occur.

The term “substituted” means that a hydrogen atom on a molecule has been replaced with a different atom or molecule. The atom or molecule replacing the hydrogen atom is denoated as “substituent.”

Formula 1 of the invention is set forth in detail as follows.

The invention is a compound of Formula 1 comprising:

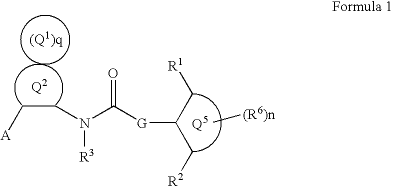

a pharmaceutically acceptable salt, solvate, or physiologically functional derivative thereof.

A in Formula 1 is selected from the group consisting of C(═O)NQ³Q⁴ and C(═O)OH.

In Formula 1, Q¹ and Q² are fused together. Q¹ is (i) a substituted or unsubstituted 5- or 6-membered aromatic ring, (ii) a substituted or unsubstituted 5- or 6-membered cycloalkyl ring, (iii) a substituted or unsubstituted 5- or 6-membered heteroaromatic ring having at least one heteroatom (and up to 4 heteroatoms) selected from the group consisting of nitrogen, oxygen, and sulfur, and (iv) a substituted or unsubstituted 4- to 8-membered heterocyclic ring having at least one heteroatom (and up to 4 heteroatoms) selected from the group consisting of nitrogen, oxygen, and sulfur. And, in Formula 1, q is 0 or 1. Preferably, Q¹ is a thienyl, a pyridyl, or a phenyl group. Most preferably, Q¹ is phenyl.

When Q¹ is (i) a 5- or 6-membered aromatic ring, (ii) a 5- or 6-membered cycloalkyl ring, (iii) a heteroaromatic ring having at least one heteroatom (and up to 4 heteroatoms) selected from the group consisting of nitrogen, oxygen, and sulfur, or (iv) a 4- to 8-membered heterocyclic ring having at least one heteroatom (and up to 4 heteroatoms) selected from the group consisting of nitrogen, oxygen, and sulfur, Q¹ can be substituted with a moiety selected from acyl; alkyl; alkenyl; alkynyl; alkylsulfonyl; alkoxy; cyano; halogen; haloalkyl; hydroxyl; —CO₂H; CO₂R^{a}; —R^{a}OH; —NR^{a}R^{b}; —CONR^{a}R^{b}; —NR^{a}SO₂R^{d}, —NR^{a}COR^{c}; —SO₂NR^{a}COR^{c}; —SO₂NR^{a}R^{b}; and —CONR^{a}SO₂R^{d} wherein each of R^{a}, R^{b}, R^{c}, and R^{d} independently are selected from the group consisting of hydrogen, alkyl, and cycloalkyl.

Q² of Formula 1 is (i) a substituted or unsubstituted 5- or 6-membered aromatic ring or (ii) a 5- or 6-membered substituted or unsubstituted heteroaromatic ring having at least one heteroatom (and up to 4 heteroatoms) selected from the group consisting of nitrogen, oxygen, and sulfur. When Q² is substituted, and q is 1, the preferred substituted moiety is alkoxy or halo. Preferably, Q² is an unsubstituted aromatic ring, a methoxysubstituted aromatic ring, or a mono- or dihalosubstituted aromatic ring. Preferably, when Q² is a heteroaromatic ring, the preferred heteroatom is N or S. Most preferred is an unsubstituted aromatic ring, in which Q² is phenyl and q is 1;

When Q² is (i) a 5- or 6-membered aromatic ring, or (ii) a 5- or 6-membered heteroaromatic ring having at least one heteroatom (and up to 4 heteroatoms) selected from the group consisting of nitrogen, oxygen, and sulfur, it can be substituted with halogen; alkoxy; alkyl; acyl; alkenyl; alkynyl; alkylsulfonyl; cyano; haloalkyl; hydroxyl; cycloalkyl, which may be further substituted with acyl, alkoxy, alkyl, alkylsulfonyl, cyano, halogen, haloalkyl, hydroxyl; heterocyclyl, which may be further substituted with acyl, alkoxy, alkyl, alkylsulfonyl, cyano, halogen, haloalkyl, hydroxyl, or nitro; aryl, which may be further substituted with acyl, alkoxy, alkyl, alkylsulfonyl, cyano, halogen, haloalkyl, hydroxyl, or nitro; heteroaryl which may be further substituted with acyl, alkoxy, alkyl, alkysulfonyl, cyano, halogen, haloalkyl, hydroxyl, or nitro; —CO₂H; —CO₂R^{a}; —R^{a}OH; —NR^{a}R^{b}; —CONR^{a}R^{b}; —NR^{a}SO₂R^{d}, —NR^{a}COR^{c}; —SO₂NR^{a}COR^{c}; —SO₂NR^{a}R^{b}; and —CONR^{a}SO₂R^{d} where each of R^{a}, R^{b}, R^{c}, and R^{d} independently are selected from the group consisting of hydrogen, alkyl, cycloalkyl, aryl, heteroaryl, and heterocyclyl.

When Q² is (i) a 5- or 6-membered aromatic ring, or (ii) a 5- or 6-membered heteroaromatic ring having at least one heteroatom (and up to 4 heteroatoms) selected from the group consisting of nitrogen, oxygen, and sulfur, and q is 0, Q² can be substituted with a moiety selected from acyl; alkyl; alkenyl; alkynyl; aryl; heteroaryl; cycloalkyl, heterocyclic; alkylsulfonyl; alkoxy; cyano; halogen; haloalkyl; hydroxyl; —CO₂H; CO₂R^{a}; —R^{a}OH; —NR^{a}R^{b}; —CONR^{a}R^{b}; —NR^{a}SO₂R^{d}, —NR^{a}COR^{c}; —SO₂NR^{a}COR^{c}; —SO₂NR^{a}R^{b}; and —CONR^{a}SO₂R^{d} wherein each of R^{a}, R^{b}, R^{c}, and R^{d} independently are selected from the group consisting of hydrogen, alkyl, cycloalkyl, aryl, heteroaryl, and aryloxy. Preferably, when q is 0, Q² is an substituted phenyl, pyridyl, or thienyl containing one or more halo groups, particularly chloro and fluoro groups, or a substituted phenyl, pyridyl, or thienyl containing one or more alkyl groups, particularly methyl, or a substituted phenyl, pyridyl, or thienyl containing one aryl group, particularly a substituted phenyl.

In Formula 1, R¹ and R² are each independently selected from the group consisting of (i) hydrogen, (ii) substituted or unsubstituted C₁₋₆ alkyl, (iii) halo (Cl, Br, I, and F), (iv) substituted or unsubstituted alkoxy, (v) monoalkylamino, and (vi) dialkylamino. Preferably, R¹ and R² are each independently selected from the group consisting of halo and C₁₋₆ alkyl. When R¹ or R² is a C₁₋₆ alkyl or alkoxy, said alkyl or alkoxy may contain a halogen group. A most preferred combination occurs when R¹ is chloro and R² is methyl or vice versa, when R¹ and R² are both chloro or both methyl. In Formula 1, R¹ and R² are each in the ortho position with respect to G.

R³ of Formula I can be (i) hydrogen or (ii) a substituted or unsubstituted C₁₋₆ alkyl. Preferably, R³ is hydrogen.

In Formula 1, Q³ and Q⁴ are each independently selected from the group consisting of (i) hydrogen, (ii) a substituted or unsubstituted C₁₋₆ alkyl, (iii) —CR⁴R⁵Z, where Z is a 5- or 6-membered substituted or unsubstituted heteroaryl having at least one heteroatom (and up to 4 heteroatoms) selected from the group consisting of nitrogen, oxygen, and sulfur, (iv) substituted or unsubstituted aryl, and (v) —CR⁴R⁵COOH. Preferably, Q³ and Q⁴ are each independently selected from the group consisting of (i) —CR⁴R⁵COOH and (ii) hydrogen. Most preferred combinations are when Q³ is —CR⁴R⁵COOH and Q⁴ is hydrogen in which R⁴ and R⁵ are as defined herein.

When Q³ or Q⁴ is (ii) a C₁₋₆ alkyl, it can be substituted with alkyl; acyl; alkenyl, alkynyl, alkylsulfonyl; alkoxy; cyano; halogen; haloalkyl; hydroxyl; alkylthio; guanidino; cycloalkyl, which may be further substituted with acyl, alkoxy, alkyl, alkylsulfonyl, cyano, halogen, haloalkyl, hydroxyl; heterocyclyl, which may be further substituted with acyl, alkoxy, alkyl, cyano, halogen, haloalkyl, hydroxyl, or nitro; aryl, which may be further substituted with acyl, alkoxy, alkyl, alkylsulfonyl, cyano, halogen, haloalkyl, hydroxyl; heteroaryl which may be further substituted with acyl, alkoxy, alkyl, alkysulfonyl, cyano, halogen, haloalkyl, hydroxyl, or nitro; —CO₂H; CO₂R^{a}, —R^{a}OH; —NR^{a}R^{b}, —CONR^{a}R^{b}; —NR^{a}SO₂R^{d}, —NR^{a}COR^{c}; —SO₂NR^{a}COR^{c}; —SO₂NR^{a}R^{b}; and —CONR^{a}SO₂R^{d} where each of R^{a}, R^{b}, R^{c}, and R^{d} independently are selected from the group consisting of hydrogen, or alkyl.

When Q³ or Q⁴ is (iii) —CR⁴R⁵Z, where Z is a 5- or 6-membered heteroaryl having at least one heteroatom (and up to 4 heteroatom) selected from the group consisting of nitrogen, oxygen, and sulfur, or (iv) aryl, said Q³ and Q⁴ can be substituted with alkyl; acyl; alkenyl, alkynyl, alkylsulfonyl; alkoxy; cyano; halogen; haloalkyl; hydroxyl; alkylthio; —CO₂H; —R^{a}OH; —CO₂R^{a}; —NR^{a}R^{b}; —CONR^{a}R^{b}; —NR^{a}SO₂R^{d}, —NR^{a}COR^{c}; —SO₂NR^{a}COR^{c}; —SO₂NR^{a}R^{b}; —CONR^{a}SO₂R^{d} or —NR^{a}R^{b} where each of R^{a}, R^{b}, R^{c}, and R^{d} independently are selected from the group consisting of hydrogen, or alkyl.

In Formula 1, R⁴ and R⁵ are each independently selected from the group consisting of (i) hydrogen, (ii) a substituted or unsubstituted C₁₋₆ alkyl, (iii) a 4- to 8-membered substituted or unsubstituted cycloalkyl, (iv) a 5- or 6-membered substituted or unsubstituted aryl, (v) a 5- or 6-membered substituted or unsubstituted heteroaryl, (vi) a 5- or 6-membered substituted or unsubstituted aralkyl, (vii) a 5- or 6-membered substituted or unsubstituted heteroaralkyl, having at least one heteroatom (and up to 4 heteroatoms) selected from the group consisting of nitrogen, oxygen, and sulfur, (viii) a 4- to 8-membered substituted or unsubstituted cycloalkylalkyl, and (ix) a 4- to 8-membered substituted or unsubstituted heterocyclic ring. Preferably, R⁴ and R⁵ are selected from the group consisting of (i) hydrogen, (ii) cycloalkyl, (iii) aryl, (iv) substituted or unsubstituted C₁₋₆ alkyl, and (v) aralkyl. Most preferably, R⁴ and R⁵ are selected from the group consisting of hydrogen, aryl, cycloalkyl, and substituted C₁₋₆ alkyl, which alkyl is optionally substituted with alkoxy or —CO₂H.

When R⁴ or R⁵ is (ii) a substituted or unsubstituted C₁₋₆ alkyl, said R⁴ and R⁵ can be substituted with alkyl; acyl; alkenyl, alkynyl, alkylsulfonyl; alkoxy; cyano; halogen; haloalkyl; hydroxyl; alkylthio; guanidino; cycloalkyl, which may be further substituted with acyl, alkoxy, alkyl, alkylsulfonyl, cyano, halogen, haloalkyl, hydroxyl; heterocyclyl, which may be further substituted with acyl, alkoxy, alkyl, cyano, halogen, haloalkyl, hydroxyl, or nitro; aryl, which may be further substituted with acyl, alkoxy, alkyl, alkylsulfonyl, cyano, halogen, haloalkyl, hydroxyl; heteroaryl which may be further substituted with acyl, alkoxy, alkyl, alkysulfonyl, cyano, halogen, haloalkyl, hydroxyl, or nitro; —CO₂H; CO₂R^{a}, —R^{a}OH; —NR^{a}R^{b}, —CONR^{a}R^{b}; —NR^{a}SO₂R^{d}, —NR^{a}COR^{c}; —SO₂NR^{a}R^{b}; —SO₂NR^{a}COR^{c}; and —CONR^{a}SO₂R^{d} where each of R^{a}, R^{b}, R^{c}, and R^{d} independently are selected from the group consisting of hydrogen and alkyl.

When R⁴ or R⁵ is (iii) a 4- to 8-membered cycloalkyl, (iv) a 5- or 6-membered aryl, (v) a 5- or 6-membered heteroaryl, (vi) a 5- or 6-membered aralkyl, (vii) a 5- or 6-membered heteroaralkyl, having at least one heteroatom selected from the group consisting of nitrogen, oxygen, and sulfur, (viii) a 4- to 8-membered cycloalkylalkyl, or (ix) a 4- to 8-membered heterocyclic ring, said R⁴ and said R⁵ can be substituted with hydroxyl; halogen; alkyl; acyl; alkylsulfonyl; alkoxy; cyano; haloalkyl; alkylthio; —CO₂H; CO₂R^{a}, —R^{a}OH; —NR^{a}R^{b}, —CONR^{a}R^{b}; —NR^{a}SO₂R^{d}, —NR^{a}COR^{c}; —SO₂NR^{a}COR^{c}; —SO₂NR^{a}R^{b}; and —CONR^{a}SO₂R^{d} where each of R^{a}, R^{b}, R^{c}, and R^{d} independently are selected from the group consisting of hydrogen and alkyl.

In Formula 1, R⁴ and R⁵ taken together can form a (i) 3-10 membered cycloalkyl or (ii) a 4-8 membered heterocyclic ring.

When R⁴ and R⁵ taken together form a (i) 3-10 membered cycloalkyl or (ii) a 4-8 membered heterocyclic ring, said ring can be substituted with hydroxyl; halogen; alkyl; acyl; alkylsulfonyl; alkoxy; cyano; haloalkyl; alkylthio; —CO₂H; CO₂R^{a}, —R^{a}OH; —NR^{a}R^{b}, —CONR^{a}R^{b}; —NR^{a}SO₂R^{d}, —NR^{a}COR^{c}; —SO₂NR^{a}COR^{c}; —SO₂NR^{a}R^{b}; and —CONR^{a}SO₂R^{d}, where each of R^{a}, R^{b}, R^{c}, and R^{d} independently are selected from the group consisting of hydrogen and alkyl.

G is selected from the group consisting of carbon, nitrogen, oxygen, and sulfur. Preferably in Formula 1, G is carbon or nitrogen.

Q⁵ of Formula I is (i) a substituted or unsubstituted 5- or 6-membered aromatic ring, or (ii) a 5- or 6-membered substituted or unsubstituted heteroaromatic ring having at least one heteroatom (and up to 4 heteroatoms) selected from the group consisting of nitrogen, oxygen, and sulfur. When Q⁵ is (i) a 5- or 6-membered aromatic ring or (ii) a 5- or 6-membered heteroaromatic ring having at least one heteroatom (and up to 4 heteroatoms) selected from the group consisting of nitrogen, oxygen, and sulfur, said Q⁵ can be substituted with R¹, R² and/or R⁶ as defined herein. Preferably Q⁵ is a substituted or unsubstituted 6-membered aromatic ring. Most preferably, Q⁵ is substituted phenyl.

Optionally, Q5 can have an additional substituent R6 in any of the remaining positions (that is, the non-ortho positions relative to G). This is denoted by (R6)n, where n is 0 or 1. In Formula 1, when R6 is present (that is when n equals 1), R6 is selected from the group consisting of (i) substituted or unsubstituted CI-6 alkyl, (ii) halogen, (iii) alkoxy, (iv) cyano, (v) hydroxyl, (vi) haloalkyl, (vii) mono- or dialkylamino, (viii) 3-5 membered cycloalkyl, (ix) 3-5 membered cycloalkylalkyl, (x) alkenyl, (xi) alkynyl, and (xii) acyl;. When R6 is a C1-6 alkyl, it can be substituted with halogen. Preferably, R6 is C1-3 alkyl, trihalomethyl, cycloalkylalkyl, trifluoromethoxy or halo. Most preferably R6 is in the para position with respect to G.

As used herein throughout the present specification, the phrase “optionally substituted” or variations thereof denote an optional substitution, including multiple degrees of substitution, with one or more substituent groups. The phrase should not be interpreted so as to be imprecise or duplicative of substitution patterns herein described or depicted specifically. Rather, those of ordinary skill in the art will appreciate that the phrase is included to provide for obvious modifications, which are encompassed within the scope of the appended claims.

Specific compounds of Formula 1 include but are not limited to those set forth in Table 1 below and/or those prepared in the examples herein.

Preferred compounds according to the invention are
N-[3-({[(2,6-dimethylphenyl)amino]carbonyl}amino)-2-naphthoyl]glycine;
Phenyl({[3-({[(2,4,6-trimethylphenyl)amino]carbonyl}amino)-2-naphthalenyl]carbonyl}amino)acetic acid;
(2S)-Cyclohexyl({[3-({[(2,6-dichlorophenyl)amino]carbonyl}amino)-2-naphthalenyl]carbonyl}amino)acetic acid;
(2S)({[4-chloro-2-({[(2,6-dichlorophenyl)amino]carbonyl}amino)phenyl]carbonyl}amino)(cyclohexyl)ethanoic acid;
(2S)-Cyclohexyl{[3-({[(2,4,6-trichlorophenyl)amino]carbonyl}amino)-2-naphthoyl]amino}ethanoic acid;
(2S)-Cyclohexyl{[3-({[(2-ethyl-6-methylphenyl)amino]carbonyl}amino)-2-naphthoyl]amino}ethanoic acid;
(2S)-({3-[({[2-Chloro-6-(trifluoromethyl)phenyl]amino}carbonyl)amino]-2-naphthoyl}amino)(cyclohexyl)ethanoic acid;
(2S)-Cyclohexyl[(3-{[(2,4,6-trichlorophenyl)acetyl]amino}-2-naphthoyl)amino]ethanoic acid
(2S)-Cyclohexyl[(3-{[(mesitylamino)carbonyl]amino}-2-naphthoyl)amino]ethanoic acid;
(2S)-Cyclohexyl({[4,5-dichloro-2-({[(2,6-dichlorophenyl)amino]carbonyl}amino)phenyl]carbonyl}amino)ethanoic acid; and
(2S)-({[4-Chloro-2-({[(2,4,6-trimethylphenyl)amino]carbonyl}amino)phenyl]carbonyl}amino)(cyclohexyl)-ethanoic acid;
(2S)-Cyclohexyl({[4,5-dichloro-2-({[(2,6-dimethylphenyl)amino]carbonyl}amino)phenyl]carbonyl}amino)ethanoic acid; and
(2S)-Cyclohexyl({[2-({[(2,6-dimethylphenyl)amino]carbonyl}amino)-4-(3-pyridinyl)phenyl]carbonyl}amino)ethanoic acid;
(2S)-Cyclohexyl({[3-({[(2,4,6-trimethylphenyl)amino]carbonyl}amino)-4-biphenylyl]carbonyl}amino)ethanoic acid;
(2S)-Cyclohexyl({[2-({[(2,6-dimethylphenyl)amino]carbonyl}amino)-4-(2-thienyl)phenyl]carbonyl}amino)ethanoic acid;
(2S)-Cyclohexyl({[3-({[(2,6-dimethylphenyl)amino]carbonyl}amino)-4′-hydroxy-4-biphenylyl]carbonyl}amino)ethanoic acid;
(2S)-Cyclohexyl({[3-({[(2,6-dimethylphenyl)amino]carbonyl}amino)-3′,4′-difluoro-4-biphenylyl]carbonyl}amino)ethanoic acid;
(2S)-Cyclohexyl({[3-({[(2,6-dimethylphenyl)amino]carbonyl}amino)-4′-(methyloxy)-4-biphenylyl]carbonyl}amino)ethanoic acid;
(2S)-Cyclohexyl({[4′-(methyloxy)-3-({[(2,4,6-trimethylphenyl)amino]carbonyl}amino)-4-biphenylyl]carbonyl}amino)ethanoic acid;
(2S)-Cyclohexyl({[4′-hydroxy-3-({[(2,4,6-trimethylphenyl)amino]carbonyl}amino)-4-biphenylyl]carbonyl}amino)ethanoic acid;
(2S)-Cyclohexyl({[4′-nitro-3-({[(2,4,6-trimethylphenyl)amino]carbonyl}amino)-4-biphenylyl]carbonyl}amino)ethanoic acid;
(2S)-Cyclohexyl({[4′-(hydroxymethyl)-3-({[(2,4,6-trimethylphenyl)amino]carbonyl}amino)-4-biphenylyl]carbonyl}amino)ethanoic acid;
(2S)-({[4′-Amino-3-({[(2,4,6-trimethylphenyl)amino]carbonyl}amino)-4-biphenylyl]carbonyl}amino)(cyclohexyl)ethanoic acid;
(2S)-Cyclohexyl({[3-({[(2,6-dichlorophenyl)amino]carbonyl}amino)-4-biphenylyl]carbonyl}amino)ethanoic acid;
(2S)-Cyclohexyl({[4-{[(methylamino)carbonyl]amino}-2-({[(2,4,6-trimethylphenyl)amino]carbonyl}amino)phenyl]carbonyl}amino)ethanoic acid;
(2S)-Cyclohexyl({[3′,4′-difluoro-3-({[(2,4,6-trimethylphenyl)amino]carbonyl}amino)-4-biphenylyl]carbonyl}amino)ethanoic acid;
(2S)-Cyclopentyl({[4′-(methyloxy)-3-({[(2,4,6-trimethylphenyl)amino]carbonyl}amino)-4-biphenylyl]carbonyl}amino)ethanoic acid;
(2S)-Cyclohexyl{[(3-{[({2,6-dichloro-4-[(trifluoromethyl)oxy]phenyl}amino)carbonyl]amino}-3′,4′-difluoro-4-biphenylyl)carbonyl]amino}ethanoic acid;
(2S)-Cyclohexyl({[4′-[(dimethylamino)methyl]-3-({[(2,4,6-trimethylphenyl)amino]carbonyl}amino)-4-biphenylyl]carbonyl}amino)ethanoic acid;
(2S)-Cyclohexyl{[(3-{[({2,6-dichloro-4-[(trifluoromethyl)oxy]phenyl}amino)carbonyl]amino}-4-biphenylyl)carbonyl]amino}ethanoic acid;
(2S)-Cyclohexyl({[3-{[({2,6-dichloro-4-[(trifluoromethyl)oxy]phenyl}amino)carbonyl]amino}-4′-(methyloxy)-4-biphenylyl]carbonyl}amino)ethanoic acid;
(2S)-Cyclohexyl({[4′-(1-pyrrolidinylmethyl)-3-({[(2,4,6-trimethylphenyl)amino]carbonyl}amino)-4-biphenylyl]carbonyl}amino)ethanoic acid;
(2S)-cyclohexyl({[4′-(4-morpholinylmethyl)-3-({[(2,4,6-trimethylphenyl)amino]carbonyl}amino)-4-biphenylyl]carbonyl}amino)ethanoic acid;
(2S)-Cyclohexyl({[4′-(ethyloxy)-3-({[(2,4,6-trimethylphenyl)amino]carbonyl}amino)-4-biphenylyl]carbonyl}amino)ethanoic acid;
N-{[4′-(methyloxy)-3-({[(2,4,6-trimethylphenyl)amino]carbonyl}amino)-4-biphenylyl]carbonyl}-L-norleucine;
1-({[4′-(methyloxy)-3-({[(2,4,6-trimethylphenyl)amino]carbonyl}amino)-4-biphenylyl]carbonyl}amino)cycloheptanecarboxylic acid;
(2S)-Cyclohexyl({[4′-fluoro-3-({[(2,4,6-trimethylphenyl)amino]carbonyl}amino)-4-biphenylyl]carbonyl}amino)ethanoic acid;
(2S)-({[4-(1,3-Benzodioxol-5-yl)-2-({[(2,4,6-trimethylphenyl)amino]carbonyl}amino)phenyl]carbonyl}amino)(cyclohexyl)-ethanoic acid;
O-(1,1-Dimethylethyl)-N-{[4′-(methyloxy)-3-({[(2,4,6-trimethylphenyl)amino]carbonyl}amino)-4-biphenylyl]carbonyl}-L-threonine; and
1-({[3′,4′-Difluoro-3-({[(2,4,6-trimethylphenyl)amino]carbonyl}amino)-4-biphenylyl]carbonyl}amino)cyclooctanecarboxylic acid;
(2S)-Cyclohexyl({[4-(2,3-dihydro-1,4-benzodioxin-6-yl)-2-({[(2,4,6-trimethylphenyl)amino]carbonyl}amino)phenyl]carbonyl}amino)ethanoic acid;
(2S)-({[3′,4′-Bis(methyloxy)-3-({[(2,4,6-trimethylphenyl)amino]carbonyl}amino)-4-biphenylyl]carbonyl}amino)(cyclohexyl)ethanoic acid;
(2S)-Cyclohexyl({[4,5-difluoro-2-({[(2,4,6-trimethylphenyl)amino]carbonyl}amino)phenyl]carbonyl}amino)ethanoic acid;
1-({[4′-(Methyloxy)-3-({[(2,4,6-trimethylphenyl)amino]carbonyl}amino)-4-biphenylyl]carbonyl}amino)cyclooctanecarboxylic acid;
N-{[3-{[({2,6-Dichloro-4-[(trifluoromethyl)oxy]phenyl}amino)carbonyl]amino}-4′-(methyloxy)-4-biphenylyl]carbonyl}-O-(1,1-dimethylethyl)-L-threonine;
O-(1,1-Dimethylethyl)-N-{[3′-fluoro-3-({[(2,4,6-trimethylphenyl)amino]carbonyl}amino)-4-biphenylyl]carbonyl}-L-threonine;
(2S)-Cyclohexyl({[3′-fluoro-3-({[(2,4,6-trimethylphenyl)amino]carbonyl}amino)-4-biphenylyl]carbonyl}amino)ethanoic acid;
O-(1,1-Dimethylethyl)-N-{[3′-fluoro-4′-(methyloxy)-3-({[(2,4,6-trimethylphenyl)amino]carbonyl}amino)-4-biphenylyl]carbonyl}-L-threonine;
O-(1,1-Dimethylethyl)-N-{[3-({[(2,6-dimethyl-4-propylphenyl)amino]carbonyl}amino)-4′-(methyloxy)-4-biphenylyl]carbonyl}-L-threonine;
(2S)-Cyclohexyl({[3′-fluoro-4′-(methyloxy)-3-({[(2,4,6-trimethylphenyl)amino]carbonyl}amino)-4-biphenylyl]carbonyl}amino)ethanoic acid;
1-({[3′-Fluoro-4′-(methyloxy)-3-({[(2,4,6-trimethylphenyl)amino]carbonyl}amino)-4-biphenylyl]carbonyl}amino)cyclooctanecarboxylic acid;
N-{[3-({[(2,4,6-trimethylphenyl)amino]carbonyl}amino)-2-naphthalenyl]carbonyl}-L-norleucine;
O-(1,1-dimethylethyl)-N-{[3-({[(2,4,6-trimethylphenyl)amino]carbonyl}amino)-2-naphthalenyl]carbonyl}-L-serine;
5-methyl-N-{[3-({[(2,4,6-trimethylphenyl)amino]carbonyl}amino)-2-naphthalenyl]carbonyl}norleucine;
6,6,6-trifluoro-N-{[3-({[(2,4,6-trimethylphenyl)amino]carbonyl}amino)-2-naphthalenyl]carbonyl}norleucine;
O-(1,1-dimethylethyl)-N-{[3-({[(2,4,6-trimethylphenyl)amino]carbonyl}amino)-2-naphthalenyl]carbonyl}-L-threonine;
N-{[3-({[(2,4,6-trimethylphenyl)amino]carbonyl}amino)-2-naphthalenyl]carbonyl}-L-leucine;
N-{[3-({[(2,4,6-trimethylphenyl)amino]carbonyl}amino)-2-naphthalenyl]carbonyl}-L-isoleucine;
N-{[3-({[(2,4,6-trimethylphenyl)amino]carbonyl}amino)-2-naphthalenyl]carbonyl}-L-norvaline;
O-(1,1-dimethylethyl)-N-[(3-{[(2,4,6-trimethylphenyl)acetyl]amino}-2-naphthalenyl)carbonyl]-L-threonine;
O-butyl-N-{[3-({[(2,4,6-trimethylphenyl)amino]carbonyl}amino)-2-naphthalenyl]carbonyl}-L-serine;
O-[2-(methyloxy)ethyl]-N-{[3-({[(2,4,6-trimethylphenyl)amino]carbonyl}amino)-2-naphthalenyl]carbonyl}-L-serine;
O-ethyl-N-{[3-({[(2,4,6-trimethylphenyl)amino]carbonyl}amino)-2-naphthalenyl]carbonyl}-L-serine;
O-(1-methylethyl)-N-{[3-({[(2,4,6-trimethylphenyl)amino]carbonyl}amino)-2-naphthalenyl]carbonyl}-L-serine;
O-(2,2-dimethylpropyl)-N-{[3-({[(2,4,6-trimethylphenyl)amino]carbonyl}amino)-2-naphthalenyl]carbonyl}-L-serine;
O-(tetrahydro-2H-pyran-4-yl)-N-{[3-({[(2,4,6-trimethylphenyl)amino]carbonyl}amino)-2-naphthalenyl]carbonyl}-L-serine;
O-(1-methylethyl)-N-{[3-({[(2,4,6-trimethylphenyl)amino]carbonyl}amino)-2-naphthalenyl]carbonyl}-L-threonine;
(2S)-Cyclohexyl({[3-({[(2,4,6-trimethylphenyl)amino]carbonyl}amino)-2-quinolinyl]carbonyl}amino)ethanoic acid;
1-({[3-({[(2,4,6-trimethylphenyl)amino]carbonyl}amino)-2-naphthalenyl]carbonyl}amino)cycloheptanecarboxylic acid;
1-({[3-({[(2,4,6-trichlorophenyl)amino]carbonyl}amino)-2-naphthalenyl]carbonyl}amino)cyclooctanecarboxylic acid;
1-({[3-({[(2,4,6-trimethylphenyl)amino]carbonyl}amino)-2-naphthalenyl]carbonyl}amino)cyclooctanecarboxylic acid;
1-({[3-({[(2,4,6-trimethylphenyl)amino]carbonyl}amino)-2-naphthalenyl]carbonyl}amino)cyclodecanecarboxylic acid;
1-({[3-({[(2,4,6-trimethylphenyl)amino]carbonyl}amino)-2-quinolinyl]carbonyl}amino)cycloheptanecarboxylic acid;
1-({[3-({[(2,4,6-trimethylphenyl)amino]carbonyl}amino)-2-quinolinyl]carbonyl}amino)cyclooctanecarboxylic acid;
1-({[3-({[(2,6-dimethyl-4-propylphenyl)amino]carbonyl}amino)-2-naphthalenyl]carbonyl}amino)cycloheptanecarboxylic acid;
2-({[3-({[(2,4,6-trimethylphenyl)amino]carbonyl}amino)-2-naphthalenyl]carbonyl}amino)-2,3-dihydro-1H-indene-2-carboxylic acid;
2-({[3-({[(2,4,6-trimethylphenyl)amino]carbonyl}amino)-2-naphthalenyl]carbonyl}amino)-1,2,3,4-tetrahydro-2-naphthalenecarboxylic acid;
1-({[5-Chloro-3-({[(2,6-dimethyl-4-propylphenyl)amino]carbonyl}amino)-2-pyridinyl]carbonyl}amino)cyclooctanecarboxylic acid;
(2S)-Cyclohexyl({[5-[4-(methyloxy)phenyl]-3-({[(2,4,6-trimethylphenyl)amino]carbonyl}amino)-2-pyridinyl]carbonyl}amino)ethanoic acid;
1-({[5-[4-(methyloxy)phenyl]-3-({[(2,4,6-trimethylphenyl)amino]carbonyl}amino)-2-pyridinyl]carbonyl}amino)cycloheptanecarboxylic acid;
O-(phenylmethyl)-N-{[3-({[(2,4,6-trimethylphenyl)amino]carbonyl}amino)-2-naphthalenyl]carbonyl}-L-threonine;
(3R)-3-[(phenylmethyl)oxy]-N-{[3-({[(2,4,6-trimethylphenyl)amino]carbonyl}amino)-2-naphthalenyl]carbonyl}-L-norvaline;
(2S)-(4,4-difluorocyclohexyl)({[3-({[(2,4,6-trimethylphenyl)amino]carbonyl}amino)-2-naphthalenyl]carbonyl}amino)ethanoic acid;
(2S)-cyclopentyl({[3-({[(2,4,6-trimethylphenyl)amino]carbonyl}amino)-2-naphthalenyl]carbonyl}amino)ethanoic acid;
1,4-dioxaspiro[4.5]dec-8-yl({[3-({[(2,4,6-trimethylphenyl)amino]carbonyl}amino)-2-naphthalenyl]carbonyl}amino)acetic acid;
(cis and trans)-[4-({[(1,1-dimethylethyl)oxy]carbonyl}amino)cyclohexyl]({[3-({[(2,4,6-trimethylphenyl)amino]carbonyl}amino)-2-naphthalenyl]carbonyl}amino)acetic acid;
(cis and trans)-(4-{[(methylamino)carbonyl]amino}cyclohexyl)({[3-({[(2,4,6-trimethylphenyl)amino]carbonyl}amino)-2-naphthalenyl]carbonyl}amino)acetic acid;
N-{[3-({[(2,4,6-trimethylphenyl)amino]carbonyl}amino)-2-naphthalenyl]carbonyl}-L-aspartic acid;
N-[(3-{[({2,6-dichloro-4-[(trifluoromethyl)oxy]phenyl}amino)carbonyl]amino}-2-naphthalenyl)carbonyl]-L-aspartic acid;
N-{[3-({[(2,4,6-trimethylphenyl)amino]carbonyl}amino)-2-naphthalenyl]carbonyl}-D-aspartic acid;
(2S)-[(1S)-3-oxocyclohexyl]({[3-({[(2,4,6-trimethylphenyl)amino]carbonyl}amino)-2-naphthalenyl]carbonyl}amino)ethanoic acid;
(2S)-[(1S)-3-hydroxycyclohexyl]({[3-({[(2,4,6-trimethylphenyl)amino]carbonyl}amino)-2-naphthalenyl]carbonyl}amino)ethanoic acid;
(2S)-{(1S)-3-[(trifluoroacetyl)oxy]cyclohexyl}({[3-({[(2,4,6-trimethylphenyl)amino]carbonyl}amino)-2-naphthalenyl]carbonyl}amino)ethanoic acid;
N-{[4′-(methyloxy)-3-({[(2,4,6-trimethylphenyl)amino]carbonyl}amino)-4-biphenylyl]carbonyl}-L-aspartic acid;
(2S)-2-({[3-({[(2,6-dimethyl-4-propylphenyl)amino]carbonyl}amino)-4′-(methyloxy)-4-biphenylyl]carbonyl}amino)-4-(ethyloxy)-4-oxobutanoic acid;
N-{[3-({[(2,6-dimethyl-4-propylphenyl)amino]carbonyl}amino)-4′-(methyloxy)-4-biphenylyl]carbonyl}-L-aspartic acid;
N-{[3′,4′-difluoro-3-({[(2,4,6-trimethylphenyl)amino]carbonyl}amino)-4-biphenylyl]carbonyl}-O-(1,1-dimethylethyl)-L-threonine;
N-{[3′,4′-difluoro-3-({[(2,4,6-trimethylphenyl)amino]carbonyl}amino)-4-biphenylyl]carbonyl}-L-aspartic acid;

N²-{[4′-(methyloxy)-3-({[(2,4,6-trimethylphenyl)amino]carbonyl}amino)-4-biphenylyl]carbonyl}-L-asparagine;
N-{[3′,4′-difluoro-3-({[(2,4,6-trimethylphenyl)amino]carbonyl}amino)-4-biphenylyl]carbonyl}-L-glutamic acid;
(2S)-cyclohexyl[({3-({[(2,6-dichlorophenyl)amino]carbonyl}amino)-5-[4-(methyloxy)phenyl]-2-thienyl}carbonyl)amino]ethanoic acid;
(2S)-cyclohexyl({[5-[4-(methyloxy)phenyl]-2-({[(2,4,6-trimethylphenyl)amino]carbonyl}amino)-3-thienyl]carbonyl}amino)ethanoic acid;
(2S)-cyclohexyl[({2-{[({2,6-dichloro-4-[(trifluoromethyl)oxy]phenyl}amino)carbonyl]amino}-5-[4-(methyloxy)phenyl]-3-thienyl}carbonyl)amino]ethanoic acid;
(2S)-cyclohexyl{[(2-{[({2,6-dichloro-4-[(trifluoromethyl)oxy]phenyl}amino)carbonyl]amino}-5-{4-[(trifluoromethyl)oxy]phenyl}-3-thienyl)carbonyl]amino}ethanoic acid;
(2S)-cyclohexyl[({3-{[({2,6-dichloro-4-[(trifluoromethyl)oxy]phenyl}amino)carbonyl]amino}-5-[4-(methyloxy)phenyl]-2-thienyl}carbonyl)amino]ethanoic acid;
(2S)-cyclohexyl({[5-[4-(methyloxy)phenyl]-3-({[(2,4,6-trimethylphenyl)amino]carbonyl}amino)-2-thienyl]carbonyl}amino)ethanoic acid;
N-{[5-[4-(methyloxy)phenyl]-3-({[(2,4,6-trimethylphenyl)amino]carbonyl}amino)-2-thienyl]carbonyl}-L-valine;
N-{[5-[4-(methyloxy)phenyl]-3-({[(2,4,6-trimethylphenyl)amino]carbonyl}amino)-2-thienyl]carbonyl}-L-isoleucine;
N-{[5-[4-(methyloxy)phenyl]-3-({[(2,4,6-trimethylphenyl)amino]carbonyl}amino)-2-thienyl]carbonyl}-L-norleucine;
O-(1,1-dimethylethyl)-N-{[5-[4-(methyloxy)phenyl]-3-({[(2,4,6-trimethylphenyl)amino]carbonyl}amino)-2-thienyl]carbonyl}-L-serine;
O-(1,1-dimethylethyl)-N-{[5-[4-(methyloxy)phenyl]-3-({[(2,4,6-trimethylphenyl)amino]carbonyl}amino)-2-thienyl]carbonyl}-L-threonine;
1-{[5-[4-(methyloxy)phenyl]-3-({[(2,4,6-trimethylphenyl)amino]carbonyl}amino)-2-thienyl]carbonyl}-L-proline;
1-({[5-[4-(methyloxy)phenyl]-3-({[(2,4,6-trimethylphenyl)amino]carbonyl}amino)-2-thienyl]carbonyl}amino)cyclopentanecarboxylic acid;
1-({[5-[4-(methyloxy)phenyl]-3-({[(2,4,6-trimethylphenyl)amino]carbonyl}amino)-2-thienyl]carbonyl}amino)cyclohexanecarboxylic acid;
1-({[5-[4-(methyloxy)phenyl]-3-({[(2,4,6-trimethylphenyl)amino]carbonyl}amino)-2-thienyl]carbonyl}amino)cycloheptanecarboxylic acid;
1-({[5-[4-(methyloxy)phenyl]-3-({[(2,4,6-trimethylphenyl)amino]carbonyl}amino)-2-thienyl]carbonyl}amino)cyclooctanecarboxylic acid;
(2S)-cyclohexyl({[3-({[(2,6-dichloro-4-fluorophenyl)amino]carbonyl}amino)-2-naphthalenyl]carbonyl}amino)ethanoic acid;
(2S)-cyclohexyl{[(3-{[(2,4,6-trimethylphenyl)acetyl]amino}-2-naphthalenyl)carbonyl]amino}ethanoic acid;
(2S)-cyclohexyl({[3-({[(4-ethyl-2,6-dimethylphenyl)amino]carbonyl}amino)-2-naphthalenyl]carbonyl}amino)ethanoic acid;
(2S)-cyclohexyl{[(3-{[({2,6-dichloro-4-[(trifluoromethyl)oxy]phenyl}amino)carbonyl]amino}-2-naphthalenyl)carbonyl]amino}ethanoic acid;
(2S)-(trans-4-methylcyclohexyl)({[3-({[(2,4,6-trichlorophenyl)amino]carbonyl}amino)-2-naphthalenyl]carbonyl}amino)ethanoic acid;
2-cyclohexyl-N-{[3-({[(2,4,6-trimethylphenyl)amino]carbonyl}amino)-2-naphthalenyl]carbonyl}-L-alanine;
2-cyclohexyl-N-[(3-{[({2,6-dichloro-4-[(trifluoromethyl)oxy]phenyl}amino)carbonyl]amino}-2-naphthalenyl)carbonyl]-L-alanine;
{[(3-{[({2,6-dichloro-4-[(trifluoromethyl)oxy]phenyl}amino)carbonyl]amino}-2-naphthalenyl)carbonyl]amino}[trans-4-(trifluoromethyl)cyclohexyl]acetic acid;
{[(3-{[({2,6-dichloro-4-[(trifluoromethyl)oxy]phenyl}amino)carbonyl]amino}-2-naphthalenyl)carbonyl]amino}[cis-4-(trifluoromethyl)cyclohexyl]acetic acid;
{[(3-{[({2,6-dichloro-4-[(trifluoromethyl)oxy]phenyl}amino)carbonyl]amino}-2-naphthalenyl)carbonyl]amino}(tetrahydro-2H-pyran-4-yl)acetic acid;
tetrahydro-2H-pyran-4-yl({[3-({[(2,4,6-trimethylphenyl)amino]carbonyl}amino)-2-naphthalenyl]carbonyl}amino)acetic acid;
(2S)-cyclohexyl({[3-({[(2,6-dimethyl-4-propylphenyl)amino]carbonyl}amino)-2-naphthalenyl]carbonyl}amino)ethanoic acid;
(2S)-cyclohexyl[({3-[({[2,6-dimethyl-4-(2-propyn-1-yl)phenyl]amino}carbonyl)amino]-2-naphthalenyl}carbonyl)amino]ethanoic acid;
(2S)-cyclohexyl[({3-[({[2,6-dimethyl-4-(propyloxy)phenyl]amino}carbonyl)amino]-2-naphthalenyl}carbonyl)amino]ethanoic acid;
(2S)-cyclohexyl({[2-({[(4-ethyl-2,6-dimethylphenyl)amino]carbonyl}amino)-4-fluorophenyl]carbonyl}amino)ethanoic acid;
(2S)-cyclohexyl[({2-[({[2,6-dimethyl-4-(2-propen-1-yl)phenyl]amino}carbonyl)amino]-4-fluorophenyl}carbonyl)amino]ethanoic acid;
(2S)-cyclohexyl({[2-({[(2,6-dimethyl-4-propylphenyl)amino]carbonyl}amino)-4-fluorophenyl]carbonyl}amino)ethanoic acid;
(2S)-cyclohexyl({[2-({[(2,6-dimethyl-4-pentylphenyl)amino]carbonyl}amino)-4-fluorophenyl]carbonyl}amino)ethanoic acid;
2-cyclohexyl-N-{[2-({[(2,6-dimethyl-4-propylphenyl)amino]carbonyl}amino)-4-fluorophenyl]carbonyl}-L-alanine;
(2S)-({[2-({[(4-butyl-2,6-dimethylphenyl)amino]carbonyl}amino)-4-fluorophenyl]carbonyl}amino)(cyclohexyl)ethanoic acid;
O-(1,1-dimethylethyl)-N-{[3-({[(2,6-dimethyl-4-propylphenyl)amino]carbonyl}amino)-3′,4′-difluoro-4-biphenylyl]carbonyl}-L-threonine;
(2S)-cyclohexyl[({2-[({[4-(cyclopropylmethyl)-2,6-dimethylphenyl]amino}carbonyl)amino]-4-fluorophenyl}carbonyl)amino]ethanoic acid;
N-({3-[({[4-(cyclopropylmethyl)-2,6-dimethylphenyl]amino}carbonyl)amino]-3′,4′-difluoro-4-biphenylyl}carbonyl)-O-(1,1-dimethylethyl)-L-threonine;
1-({[2-[4-(Methyloxy)phenyl]-5-({[(2,4,6-trimethylphenyl)amino]carbonyl}amino)-1,3-thiazol-4-yl]carbonyl}amino)cyclohexanecarboxylic acid;
(2S)-(4-hydroxyphenyl)({[3-({[(2,4,6-trimethylphenyl)amino]carbonyl}amino)-2-naphthalenyl]carbonyl}amino)ethanoic acid;
(2S)-(4-hydroxycyclohexyl)({[3-({[(2,4,6-trimethylphenyl)amino]carbonyl}amino)-2-naphthalenyl]carbonyl}amino)ethanoic acid;

N⁴,N⁴-dimethyl-N²-{[4′-(methyloxy)-3-({[(2,4,6-trimethylphenyl)amino]carbonyl}amino)-4-biphenylyl]carbonyl}-L-asparagine;
N-({3-[({[4-(cyclopropylmethyl)-2,6-dimethylphenyl]amino}carbonyl)amino]-3′-fluoro-4-biphenylyl}carbonyl)-O-(1,1-dimethylethyl)-L-threonine;
N-{[3′-fluoro-3-({[(2,4,6-trimethylphenyl)amino]carbonyl}amino)-4-biphenylyl]carbonyl}-L-aspartic acid;
O-(Phenylmethyl)-N-{[3-({[(2,4,6-trimethylphenyl)amino]carbonyl}amino)-2-naphthalenyl]carbonyl}-L-serine;
N-{[3′,4′-Difluoro-3-({[(2,4,6-trimethylphenyl)amino]carbonyl}amino)-4-biphenylyl]carbonyl}-O-(phenylmethyl)-L-serine;
(3R)-5-Methyl-3-[(phenylmethyl)oxy]-N-{[3-({[(2,4,6-trimethylphenyl)amino]carbonyl}amino)-2-naphthalenyl]carbonyl}-L-norleucine;
O-cyclobutyl-N-{[3-({[(2,4,6-trimethylphenyl)amino]carbonyl}amino)-2-naphthalenyl]carbonyl}-L-threonine;
N-{[3-({[(2,4,6-trimethylphenyl)amino]carbonyl}amino)-2-naphthalenyl]carbonyl}-L-phenylalanine;
(2S)-4-({[(1,1-dimethylethyl)oxy]carbonyl}amino)-2-({[3-({[(2,4,6-trimethylphenyl)amino]carbonyl}amino)-2-naphthalenyl]carbonyl}amino)butanoic acid;
5,5-dimethyl-N-{[3-({[(2,4,6-trimethylphenyl)amino]carbonyl}amino)-2-naphthalenyl]carbonyl}norleucine;
O-cyclobutyl-N-{[3′,4′-difluoro-3-({[(2,4,6-trimethylphenyl)amino]carbonyl}amino)-4-biphenylyl]carbonyl}-L-threonine;
O-(1-methylcyclopentyl)-N-{[3-({[(2,4,6-trimethylphenyl)amino]carbonyl}amino)-2-naphthalenyl]carbonyl}-L-threonine;
(2S)-cyclohexyl({[2′-(methyloxy)-3-({[(2,4,6-trimethylphenyl)amino]carbonyl}amino)-4-biphenylyl]carbonyl}amino)ethanoic acid;
O-(1,1-Dimethylethyl)-N-{[2′-(methyloxy)-3-({[(2,4,6-trimethylphenyl)amino]carbonyl}amino)-4-biphenylyl]carbonyl}-L-threonine;
N-{[3′,5′-Difluoro-3-({[(2,4,6-trimethylphenyl)amino]carbonyl}amino)-4-biphenylyl]carbonyl}-O-(1,1-dimethylethyl)-L-threonine;
(2S)-Cyclohexyl({[3′,5′-difluoro-3-({[(2,4,6-trimethylphenyl)amino]carbonyl}amino)-4-biphenylyl]carbonyl}amino)ethanoic acid;
O-(1,1-Dimethylethyl)-N-{[4′-fluoro-3-({[(2,4,6-trimethylphenyl)amino]carbonyl}amino)-4-biphenylyl]carbonyl}-L-threonine;
O-(1,1-Dimethylethyl)-N-{[3-({[(2,4,6-trimethylphenyl)amino]carbonyl}amino)-4-biphenylyl]carbonyl}-L-threonine;
1-({[3-({[(2,4,6-Trimethylphenyl)amino]carbonyl}amino)-4-biphenylyl]carbonyl}amino)cyclooctanecarboxylic acid;
N-{[3-({[(4-Cyclopropyl-2,6-dimethylphenyl)amino]carbonyl}amino)-3′-fluoro-4-biphenylyl]carbonyl}-O-(1,1-dimethylethyl)-L-threonine;
(2S)-cyclohexyl({[3-({[(4-cyclopropylphenyl)amino]carbonyl}amino)-2-naphthalenyl]carbonyl}amino)ethanoic acid;
N-{[3-({[(4-cyclopropyl-2,6-dimethylphenyl)amino]carbonyl}amino)-4′-(methyloxy)-4-biphenylyl]carbonyl}-O-(1,1-dimethylethyl)-L-threonine;
1-({[5-(4-chlorophenyl)-3-({[(2,4,6-trimethylphenyl)amino]carbonyl}amino)-2-thienyl]carbonyl}amino)cyclohexanecarboxylic acid; and
1-({[5-(3,4-difluorophenyl)-3-({[(2,4,6-trimethylphenyl)amino]carbonyl}amino)-2-thienyl]carbonyl}amino)cyclohexanecarboxylic acid.

It will be appreciated by those skilled in the art that the compounds of the present invention may also be utilized in the form of a pharmaceutically acceptable salt, or solvate, or physiologically functional derivative thereof.

The pharmaceutically acceptable salts of the compounds of Formula 1 include conventional salts formed from pharmaceutically acceptable inorganic or organic acids or bases as well as quaternary ammonium salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumic, toluenesulfonic, methanesulfonic (mesylate), naphthalene-2-sulfonic, benzenesulfonic, hydroxynaphthoic, hydroidic, malic, steroicc, tannic, and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine, and procaine salts.

The term “physiologically functional derivative” as used herein refers to any pharmaceutically acceptable derivative of a compound of the present invention, for example, an ester or an amide of a compound of Formula 1, which upon administration to an animal, particularly a mammal, such as a human, is capable of providing (directly or indirectly) a compound of the present invention or an active metabolite thereof. See, for example, Burger's Medicinal Chemistry and Drug Discovery, 5^{th} Edition, Volume 1: Principles and Practice.

Processes for preparing pharmaceutically acceptable salts, solvates, and physiologically functional derivative of a compound of Formula 1 are conventional in the art. See, for example, Burger's Medicinal Chemistry and Drug Discovery, 5^{th} Edition, Volume 1: Principles and Practice.

As will be apparent to those skilled in the art, the processes described herein for the preparation of compounds of Formula 1, certain intermediates, may be in the form of pharmaceutically acceptable salts, solvates, or physiologically functional derivatives of the compound. Those terms as applied to any intermediate employed in a process of preparing compounds of the invention have the same meanings as noted above with respect to compounds of Formula 1. Processes for preparing pharmaceutically acceptable salts, solvates, and physiologically functional derivatives of such intermediates are known in the art and are analogous to the process for preparing pharmaceutically acceptable salts, solvates, and physiologically functional derivatives of the compounds of Formula 1.

Certain compounds of Formula 1 may exist in stereoisomeric forms (e.g., they may contain one or more asymmetric carbon atoms or may exhibit cis-trans isomerism). The individual stereoisomers (enantiomers and diastereomers), and mixtures of these are included within the scope of the present invention. The present invention also covers the individual isomers of the compounds represented by Formula 1 as mixtures with isomers thereof in which one or more chiral centers are inverted. Certain compounds of Formula 1 may be prepared as a mixture of regioisomers. The present invention covers both the mixture of regioisomers as well as the individual compounds. Likewise, it is understood that compounds of Formula 1 may exist in tautomeric forms other than that shown in the formula and these are also included within the scope of the present invention.

It is to be understood that the present invention includes all combinations and subsets of the particular groups defined hereinabove.

Compounds of Formula 1 may be conveniently prepared by the processes outlined below. The order of the foregoing steps is not critical to the practice of the invention and the processes may be practiced by performing the steps in any suitable order based on the knowledge of those skilled in the art. The compounds of the invention can be prepared using Methods A through F described below.

Method A (Solid-Phase Synthesis of Compounds of Formula 1 using Intermediate 1 and/or 2 and/or 3 and/or 4 and/or 5.)

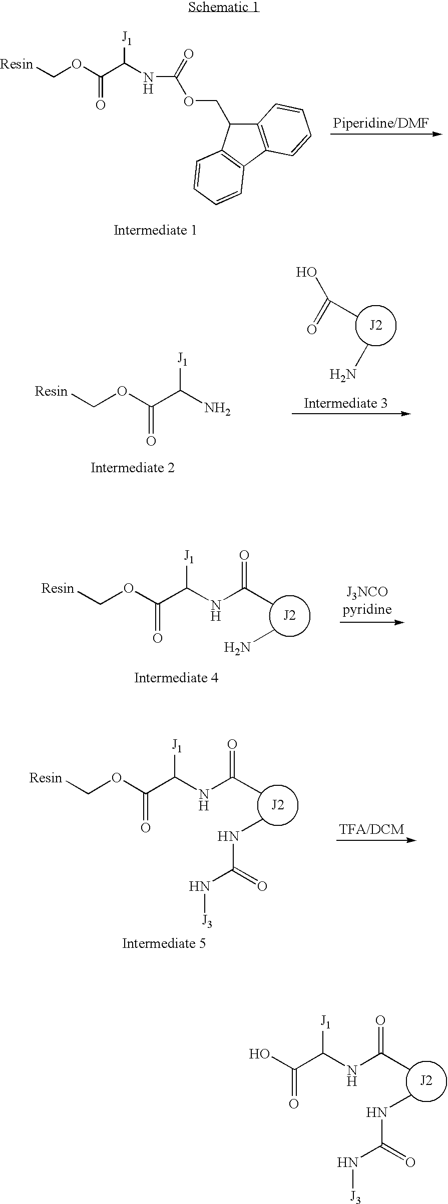

Fmoc (9-fluorenylmethoxycarbonyl) protected resin bound amino acids (e.g., Intermediate 1 in which J₁ represents various amino acid side chains) can be purchased commercially or formed by standard methods. (See, for example, Sieber, P. Tetrahedron Letters 1987, 28, 6147-6150 and references therein; and Blankemeyer-Menge, B.; Nimtz, M.; Frank, R. Tetrahedron Letters 1990, 31, 1701-1704 and references therein.) The reactions to form intermediate 2 are typically run in DMF (N,N-dimethylformamide) as a solvent, in which intermediate 1 is mixed with 20% piperidine at room temperature. Intermediate 3 (with variations at J2) can be purchased commercially or formed by standard methods. Intermediate 4 (with variations at J₁ and J2) can be formed by mixing intermediate 3 and intermediate 2 using standard coupling methods. These methods include the use of DIC (N,N′-diisopropylcarbodiimide), PyBop (Benzotriazole-1-yl-oxy-tris-pyrrolidino-phosphonium hexafluorophosphate), PyBrOP (Bromo-tris-pyrrolidino-phosphonium hexafluorophosphate), HATU (2-(1H-9-Azabenzotriazxole-1-yl)-1,1,3,3-tetramethyluronium hexafluorophosphate, or HOBT (N-hydroxybenzotriaole) at room or elevated temperature. Preferably EDC (1-ethyl-3-(3-dimethylaminopropyl)carbodiimide hydrochloride), DIEA (N,N-diisopropylethylamine) and/or HOAT (N-hydroxy-9-azabenzotriaole) are employed at room temperature. Solvents can include DMF, methylene chloride (DCM), or preferably NMP (N-methylpyrrolidinone). Intermediate 4 is then mixed with an isocyanate (e.g., J₃NCO, in which J₃ represents various side chains) in methylene chloride, diisopropylethylamine, triethylamine, or pyridine, preferably with pyridine to form intermediate 5. The reactions can be heated, but are preferably mixed at room temperature. The final product is formed by cleavage of intermediate 5 from the resin using a mixture of TFA (trifluoroacetic acid) in methylene chloride, preferably 50% TFA in DCM.

Method B (Solid-Phase Synthesis of Compounds of Formula I using Intermediate 1 and/or 2 and/or 5 and/or 6.)

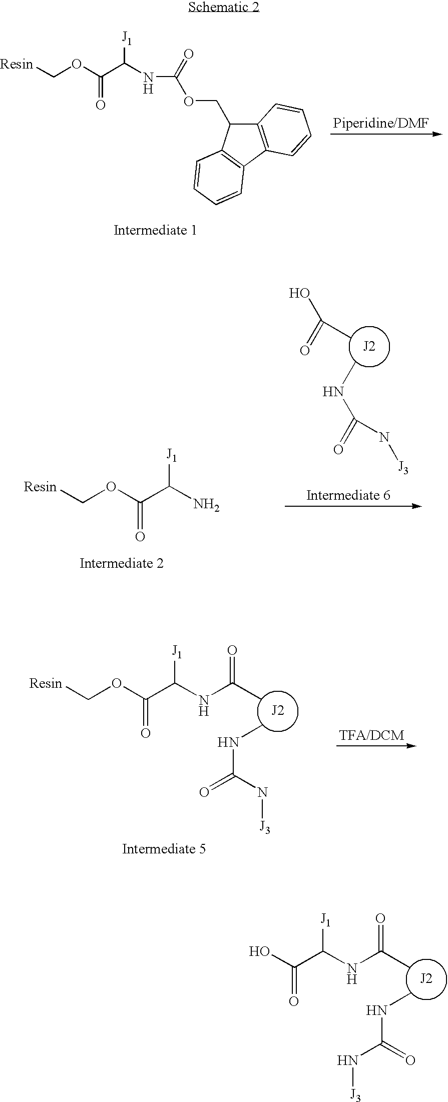

In Method B, compounds of Formula 1 can be made according to Method A, except that Intermediate 6 is used in place of intermediates 3 and 4 to form intermediate 5. Intermediate 6 can be formed by standard methods from intermediate 3 as described in Method C below.

Method C (Solution-Phase Synthesis of Compounds of Formula 1 from corresponding Intermediates 3 and/or 6.

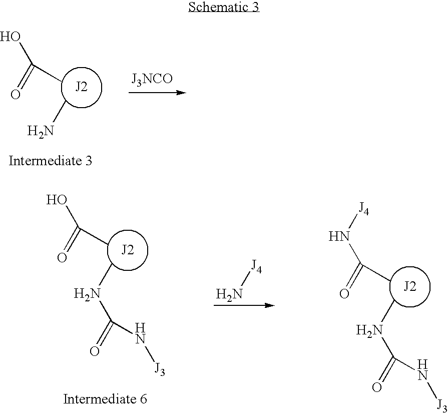

Intermediate 6 is formed by mixing intermediate 3 with an isocyanate (J₃NCO in which J₃ represents various groups) in diisopropylethylamine (DIEA), triethylamine, pyridine, DMF, or preferably DMSO (dimethylsulfoxide). The reaction is heated or preferably run at room temperature. The final product is formed using standard coupling methods by mixing intermediate 6 with an amine (J₄-NH₂ in which J₄ represents various groups) and a reagent such as EDC (1-ethyl-3-(3-dimethylaminopropyl)carbodiimide hydrochloride), PyBop (Benzotriazole-1-yl-oxy-tris-pyrrolidino-phosphonium hexafluorophosphate), PyBrOP (Bromo-tris-pyrrolidino-phosphonium hexafluorophosphate), HOBT (N-hydroxybenzotriaole), HOAT (N-hydroxy-9-azabenzotriaole), or preferably HATU (2-(1H-9-Azabenzotriazxole-1-yl)-1,1,3,3-tetramethyluronium hexafluorophosphate) or DIC (N,N′-diisopropylcarbodiimide) and DIEA (N,N-diisopropylethylamine) at room temperature. Solvents that can be used include DMF, NMP or preferably DMSO. When J₄-NH₂ contains a methyl ester in the J₄ side chain, the ester can be hydrolysed to the corresponding carboxylic acid by adding lithium hydroxide (LiOH) in solvents which include tetrahydrofuran (THF) and/or methanol (MeOH) and/or water and/or 1,4-dioxane as described in Method E.

Method D (Solid-Phase Synthesis of Compounds of Formula 1 from Intermediates 1 and/or 2 and/or 3 and/or 4). Method D is conducted according to Method A, except that acid chlorides are employed in place of isocyanates.

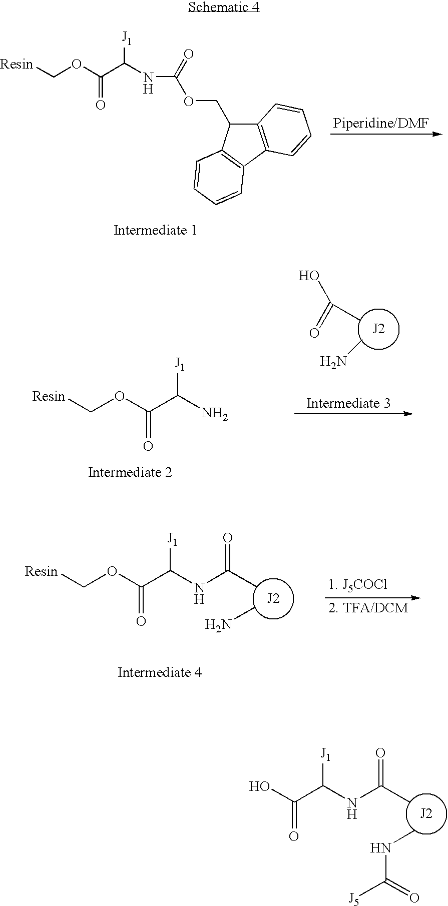

In Method D, the Fmoc (9-fluorenylmethoxycarbonyl) protected resin-bound amino acids (e.g, Intermediate 1 in which J₁ represents various amino acid side chains) can be purchased commercially or formed by standard methods. (See, for example, Sieber, P. Tetrahedron Letters 1987, 28, 6147-6150 and references therein; and Blankemeyer-Menge, B.; Nimtz, M.; Frank, R. Tetrahedron Letters 1990, 31, 1701-1704 and references therein.) The reactions to form intermediate 2 are typically run in DMF (N,N-dimethylformamide) as a solvent, in which intermediate 1 is mixed with 20% piperidine at room temperature. Intermediate 3 (with variations at J2) can be purchased commercially or formed by standard methods. Intermediate 4 (with variations at J₁ and J2) can be formed by mixing intermediate 3 and intermediate 2 using standard coupling methods. These methods include the use of DIC (N,N′-diisopropylcarbodiimide), PyBop (Benzotriazole-1-yl-oxy-tris-pyrrolidino-phosphonium hexafluorophosphate), PyBrOP (Bromo-tris-pyrrolidino-phosphonium hexafluorophosphate), HATU (2-(1H-9-Azabenzotriazxole-1-yl)-1,1,3,3-tetramethyluronium hexafluorophosphate), or HOBT (N-hydroxybenzotriaole). Preferably EDC (1-ethyl-3-(3-dimethylaminopropyl)carbodiimide hydrochloride), DIEA (N,N-diisopropylethylamine) and), and/or HOAT (N-hydroxy-9-azabenzotriaole) are employed at room temperature. Solvents can include DMF, methylene chloride (DCM), or preferably NMP (N-methylpyrrolidinone). Intermediate 4 is then mixed with an acid chloride (J₅COCl in which J₅ represents various groups) in methylene chloride, diisopropylethylamine, triethylamine, or preferably pyridine in methylene chloride. The reactions can be heated, but are preferably mixed at room temperature. The final product is then isolated by cleavage from the resin using a mixture of TFA (trifluoroacetic acid) in methylene chloride, preferably 50% TFA in DCM.

Method E (Solution-Phase Synthesis of Compounds of Formula 1 from corresponding Intermediates 3 and/or 7 and/or 8 and/or 9 and/or 10).

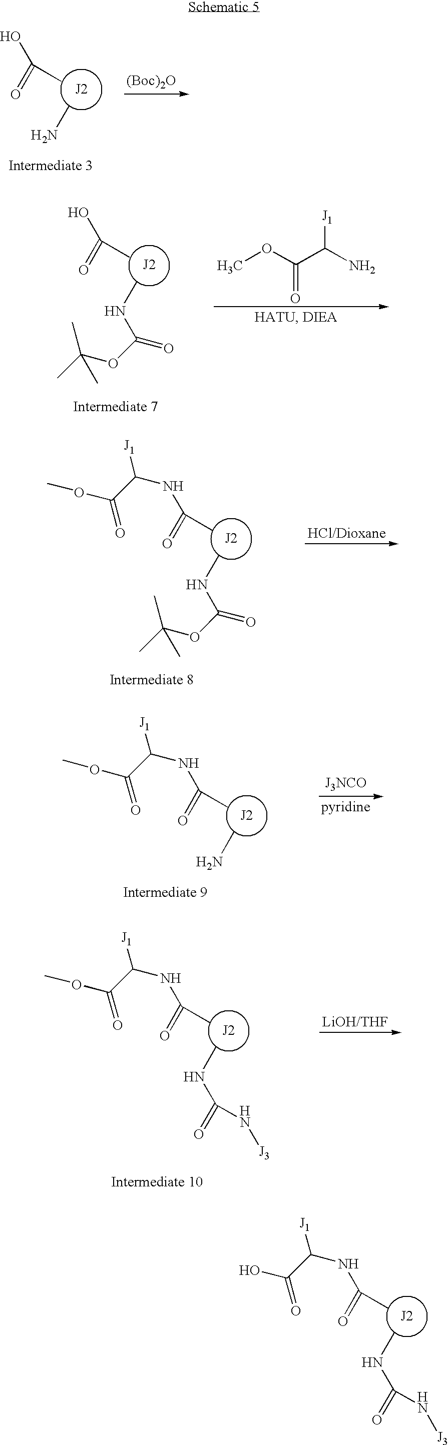

Intermediate 7 is formed by mixing Intermediate 3 with di-tert-butyl-dicarbonate ((Boc)₂O) or equivalent with an appropriate base which can include potassium hydroxide or preferably sodium hydroxide. Solvents that can be used include diethylether, dioxane, or preferably THF. The reaction is preferably run at room temperature. Intermediate 8 is formed by mixing intermediate 7 with an appropriate amine or its hydrochloride salt (NH₂CHJ₁CO₂Me in which J₁ represents various side chains) using standard coupling conditions. These conditions include the use of EDC (1-ethyl-3-(3-dimethylaminopropyl)carbodiimide hydrochloride), PyBop (Benzotriazole-1-yl-oxy-tris-pyrrolidino-phosphonium hexafluorophosphate), PyBrOP (Bromo-tris-pyrrolidino-phosphonium hexafluorophosphate), HOBT (N-hydroxybenzotriaole), HOAT (N-hydroxy-9-azabenzotriaole), or DIC (N,N′-diisopropylcarbodiimide), or preferably HATU (2-(1H-9-Azabenzotriazxole-1-yl)-1,1,3,3-tetramethyluronium hexafluorophosphate) and DIEA (N,N-diisopropylethylamine) at room temperature. Solvents that can be used include DMSO, NMP or preferably DMF. Intermediate 9 is formed by removal of the tert-butoxycarbonyl protecting group by mixing Intermediate 8 with an appropriate acid which is preferably hydrochloric acid (HCl). Solvents can include dichloromethane, diethylether, tetrahydrofuran, or preferably dioxane. The reaction is preferably mixed at room temperature.

Intermediate 9 can also be prepared directly by the reaction of intermediate 3 with an appropriate amine or its hydrochloride salt (NH₂CHJ₁CO₂Me in which J₁ represents various side chains) using standard coupling conditions. These conditions include the use of EDC (1-ethyl-3-(3-dimethylaminopropyl)carbodiimide hydrochloride), PyBop (Benzotriazole-1-yl-oxy-tris-pyrrolidino-phosphonium hexafluorophosphate), PyBrOP (Bromo-tris-pyrrolidino-phosphonium hexafluorophosphate), HOBT (N-hydroxybenzotriaole), HOAT (N-hydroxy-9-azabenzotriaole), or DIC (N,N′-diisopropylcarbodiimide), or preferably HATU (2-(1H-9-Azabenzotriazxole-1-yl)-1,1,3,3-tetramethyluronium hexafluorophosphate) and DIEA (N,N-diisopropylethylamine) at room temperature. Solvents that can be used include DMSO, NMP or preferably DMF.

Intermediate 10 is formed by mixing Intermediate 9 with an isocyanate (J₃NCO in which J₃ represents various groups) in diisopropylethylamine (DIEA), triethylamine, DMSO, DMF, or preferably pyridine. The reaction is heated or preferably mixed at room temperature.

The final product is formed by mixing Intermediate 10 with lithium hydroxide (LiOH) in solvents which include tetrahydrofuran (THF) and/or methanol (MeOH) and/or water and/or 1,4-dioxane. The reaction is preferably run at room temperature.

A simple modification of Method E is to prepare intermediate 9 directly from intermediate 3 by coupling with an appropriate amine or its hydrochloride salt (NH₂CHJ₁CO₂tBu in which J₁ represents various side chains) using standard coupling conditions. The resulting intermediate 9 as the t-butyl ester is converted to intermediate 10 (as the t-butyl ester) by treatment with an isocyanate (J₃NCO in which J₃ represents various groups) in diisopropylethylamine (DIEA), triethylamine, DMSO, DMF, or preferably pyridine. The reaction is heated or preferably mixed at room temperature. The final product is obtained by treatment with triflouroacetic acid or hydrogen chloride in solvents which include dichloromethane, tetrahydrofuran, 1,4-dioxane or ether.

Method F (Solution-Phase Synthesis of Compounds of Formula 1 from corresponding Intermediates 9 and/or 11).

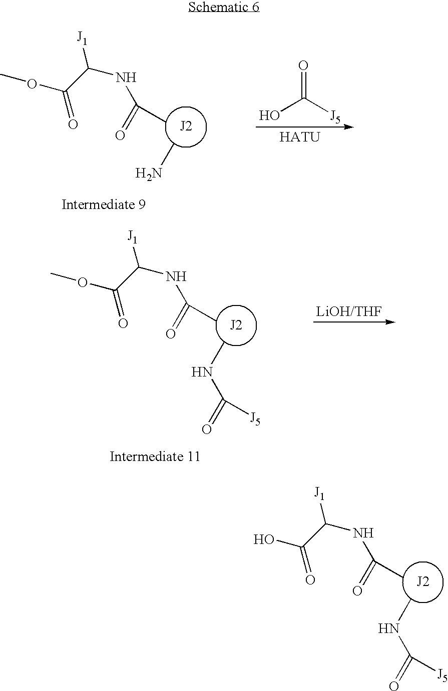

Intermediate 11 is formed by mixing Intermediate 9 (formed as described in Method E) with a carboxylic acid (J₅CO₂H in which J₅ represents various groups) using standard coupling methods (as described in Method E for the formation of intermediate 8). The carboxylic acid (J₅CO₂H in which J₅ represents various groups) can be converted to the acid chloride under standard conditions and reacted with intermediate 9 to yield intermediate 11. The final product is formed by mixing Intermediate 11 with lithium hydroxide (LiOH) as described in Method E.

In the methods outlined above (methods A-F) examples of group J₁ can be but are not limited to, side chains of natural and unnatural amino acids, modified side chains of natural amino acids such as alkyl serine and threonine, alkyl groups, cycloalkyl such as cyclohexyl and cyclopentyl, aryl groups such as phenyl, heteroaryl, alkylaryl groups such as benzyl, and spirocyclic alkyl groups. Examples of J₂ include but are not limited to aryl groups such as phenyl and substituted phenyl, naphthyl and substituted naphthyl, biphenyl and substituted biphenyl, heteroaryl such as thienyl and pyridyl, and substituted heteroaryl. Examples of J₃ include but are not limited to aryl such as phenyl and substituted phenyl such as 2,6-disubstituted phenyl and 2,4,6-trisubstituted phenyl. Examples J₄-NH₂ (method C, Schematic 3) include but are not limited to natural or unnatural amino acids containing the side chains defined by J₁, and alkyl aminobenzoates. Examples of J₅ include but are not limited to benzyl and substituted benzyl groups such as 2,6-disubstituted benzyl.

While it is possible that, for use in therapy, a therapeutically effective amount of a compound of Formula 1 may be administered as the raw chemical, it is typically presented as the active ingredient of a pharmaceutical composition or formulation. Accordingly, the invention further provides a pharmaceutical composition comprising a compound of Formula 1. The pharmaceutical composition may further comprise one or more pharmaceutically acceptable carriers, diluents, and/or excipients. The carrier(s), diluent(s), and/or excipient(s) must be acceptable in the sense of being compatible with the other ingredients of the formulation and not deleterious to the recipient thereof (that is, the patient). In accordance with another aspect of the invention there is also provided a process for the preparation of a pharmaceutical composition comprising mixing (or admixing) a compound of Formula 1 with one or more pharmaceutically acceptable carriers, diluents, and/or excipients.

Pharmaceutical compositions may be in unit dose form containing a predetermined amount of active ingredient per unit dose. Such a unit may contain a therapeutically effective dose of the compound of Formula 1 or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by the oral (including bucccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the carrier(s), diluent(s), and/or excipient(s).

When adapted for oral administration, pharmaceutical compositions may be in discrete units such as capsules or tablets; powders or granules; solutions or suspensions in aqueous or non-aqueous liquids; edible foams or whips; oil-in-water liquid emulsions or water-in-oil liquid emulsions. The compounds of the invention or pharmaceutical compositions thereof may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt, and/or an absorption agent such as bentonite, kaolin or dicalcium phosphate. The powder mixture can be granulated by wetting with a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers, such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as, for example, by coating or embedding particulate material in polymers, wax, or the like.

The present invention provides a method of treatment in a mammal, especially a human, suffering from diabetes or a related condition such as obesity, syndrome X, insulin resistance, diabetic nephropathy, diabetic neuropathy, diabetic retinopathy, hyperglycemia, hypercholesterolemia, hyperinsulinemia, hyperlipidemia, cardiovascular disease, stroke, atherosclerosis, lipoprotein disorders, hypertension, tissue ischemia, myocardial ischemia, and depression. Such treatment comprises the step of administering a therapeutically effective amount of a compound of Formula 1, including a salt, solvate, or physiologically functional derivative thereof to said mammal. Treatment can also comprise the step of administering a therapeutically effective amount of a pharmaceutical composition containing a compound of Formula 1, including a salt, solvate, or physiologically functional derivative thereof to said mammal. As used herein, the term “treatment” refers to alleviating the specified condition, eliminating or reducing the symptoms of the condition, preventing or delaying the onset of a condition, or preventing or delaying the recurrence of the condition in a previously afflicted patient or subject such as a mammal, particularly a human.

As used herein, the term “therapeutically effective amount” means an amount of a compound of Formula 1, including a salt, solvate, or physiologically functional derivative thereof or an amount of a pharmaceutical composition containing said compound of Formula 1 and/or salt, solvate, or physiologically functional derivative thereof, which amount is sufficient, in the subject or patient to which it is administered, to elicit the biological or medical response of a cell, culture, tissue, system, animal (including human), that is being sought, for instance by a researcher or clinician.

The precise therapeutically effective amount of the compounds of the invention will depend on a number of factors, including, but not limited to, the age and weight of the subject being treated, the precise disorder requiring treatment and its severity, the nature of the pharmaceutical formulation/composition, and route of administration, and will ultimately be at the discretion of the attendant physician or veterinarian. Typically, a compound of Formula 1 will be given for treatment in the range of 0.1 to 200 mg/kg body weight of recipient (animal) per day and more usually in the range of 1 to 100 mg/kg body weight per day. Acceptable daily dosages may be from about 0.1 to about 200 mg/day, and preferably from about 0.1 to about 100 mg/day.

The administration of a compound of the invention or a pharmaceutical composition containing a compound of the invention to an animal, particularly a mammal such as a human, may be by way of oral (including buccal or sub-lingual), parenteral (including subcutaneous, intramuscular, intravenous or intradermal), nasal, rectal, vaginal, or transdermal administration. Preferably, oral administration is employed.

A pharmaceutical composition of a compound of the invention may be prepared by any method known in the art of pharmacy, for example, by bringing into association the active ingredient (e.g., a compound of the invention) with one or more carriers, diluents, and/or excipients.

Additionally, the present invention comprises a compound of Formula 1, a salt, solvate, physiologically functional derivative thereof, or a pharmaceutical composition thereof with at least one other diabetic drug. Such diabetic drugs can include, for example, injected insulin and drugs such as sulfonylureas, thiazolidinediones, glipizide, glimepiride, tobutamide, acetohexamide, tolazimide, biguanides, rosiglitazone, and metformin (glucophage) and salts or combinations thereof which are ingested orally. When a compound of the invention is employed in combination with another diabetic drug, it is to be appreciated by those skilled in the art that the dose of each compound or drug of the combination may differ from that when the drug or compound is used alone. Appropriate doses will be readily appreciated and determined by those skilled in the art. The appropriate dose of the compound(s) of Formula 1 and the other therapeutically active agent(s) and the relative timings of administration will be selected in order to achieve the desired combined therapeutic effect, and are within the expertise and discretion of the attendant clinician.

### EXPERIMENTAL

The following examples are intended for illustration only and are not intended to limit the scope of the invention in any way, the invention being defined by the claims. Unless otherwise noted, reagents are commercially available or are prepared according to procedures in the literature.

Section 1: Preparation of Specific Compounds of the Invention

Chromatographic purifications of final products were carried out using reverse phase high pressure liquid chromatography, or standard silica gel chromatography unless otherwise specified. Chromatographic purification of intermediates, when necessary, was carried out using standard silica gel chromatography. Reactions were carried out in suitable containers, which can include IRORI vessels, polypropylene or teflon tubes, or glass vessels.

### Example 1

### Preparation of N-[3-({[(2,6-dimethylphenyl)amino]carbonyl}amino)-2-naphthoyl]-L-aspartic acid (Method A, Schematic 1)

### a). Preparation of L-ASP-Wang Resin

Fmoc-L-Aspartic acid tert-butyl (Asp(tBu))-Wang resin (0.8 mmol/g, obtained from Polymer Lab) (80 mg, 64 umol) in an IRORI minikan was shaken in excess 20% piperdine/DMF solution at room temperature overnight. The resin was drained, washed with DMSO (3×10 mL), DCM (3×10 mL), acetonitrile (3×10 mL), DMSO (1×10 mL), and DCM (3×10 mL). The resin was then dried in vacuo overnight to obtain L-Asp(tBu)-Wang resin as free amine.

### b). Preparation of N-[3-({[(2,6-dimethylphenyl) amino]carbonyl}amino)-2-naphthoyl]-L-aspartic acid

Diisopropyl ethyl amine (0.057 ml, 0.32 mmol) was added to the solution of EDC (1-(3-dimethylaminopropyl)-3-ethylcarbodiimide hydrochloride, 0.061 g, 0.32 mmol), HOAt (1-hydroxybenzotriazole, 0.043 g, 0.32 mmol) in N-methylpyrrolidinone, followed by the addition of IRORI minikan containing L-Asp(tBu)-Wang Resin (from Example 1a). After the reaction mixture was shaken at room temperature for 10 min, 3-amino-2-naphthalenecarboxylic acid (0.059 g, 0.32 mmol) was added to the reaction solution. The resulting reaction mixture was shaken at room temperature for 24 hours. The resin was drained, washed with DMSO (3×10 mL), DCM (3×10 mL), acetonitrile (3×10 mL), DMSO (3×10 mL), and DCM (6×10 mL), and dried in vacuo. A small portion of resin was taken out, cleaved by 1:1 TFA: DCM for 30 min. at room temperature. LC-MS showed >90% formation of desired coupling intermediate product.

The resin in the minikan was added to the solution of 2,6-dimethyl phenyl isocyanate (0.094 g, 0.64 mmol) in pyridine (20 mL). The reaction mixture was shaken at room temperature for 24 hours. The resin was drained, washed with DMSO (3×10 mL), DCM (3×10 mL), acetonitrile (3×10 mL), DMSO (3×10 mL), and DCM (6×10 mL), and dried in vacuo. The resin was then cleaved in 1:1 TFA: DCM for 30 minutes. The crude product was dried in vacuo over night, taken up by DMSO (0.6 mL), purified by HPLC, and dried in vacuo to give the title compound as a light brown solid. ESMS [M+H]+m/z 450.4.

### Example 2

### Preparation of (2S)-{[3-({[(2-chloro-6-methylphenyl)amino]carbonyl}amino)-2-naphthoyl]amino}(cyclohexyl)acetic acid (Method A, Schematic 1)

### a). Preparation of Fmoc-L-CHG-Wang Resin (Example of Intermediate 1)

Wang resin (loading 1.7 mmol/g obtained from Polymer Lab) (7.8 g, 13.3 mmol) was swollen in DMF (85 mL) for 10 minutes. To the above reaction solution, Fmoc-L-cyclohexylglycine(CHG) (10.0 g, 26.35 mmol) was added, followed by the addition of pyridine (3.4 g, 43.5 mmol), 2,6-dichlorophenyl acid chloride (5.5 g, 26.34 mmol). The reaction solution was then shaken at room temperature overnight. The resin was drained, washed with DMSO (3×100 mL), DCM (3×100 mL), acetonitrile (3×100 mL), DMSO (1×100 mL), and DCM (3×100 mL).). The resin was then dried in vacuo overnight.

### b). Preparation of L-CHG-Wang Resin (Example of Intermediate 2)

Fmoc-L-CHG-Wang resin from 2a (80 mg, 64 umol) in an IRORI minikan was shaken in excess 20% piperdine/DMF solution at room temperature overnight. The resin was drained, washed with DMSO (3×10 mL), DCM (3×10 mL), acetonitrile (3×10 mL), DMSO 1×10 mL), and DCM (3×10 mL). The resin was then dried in vacuo overnight to obtain L-CHG-Wang resin as free amine.

### c). Preparation of (2S)-{[3-({[(2-chloro-6-methylphenyl)amino]carbonyl}amino)-2-naphthoyl]amino}(cyclohexyl)acetic acid

The title compound was prepared by the same procedure as example 1 b except that L-Asp(tBu)-Wang resin was replaced with L-CHG-Wang resin (obtained from 2b) and 2,6-dimethyl phenyl isocyanate was replaced with 2-chloro-6-methyl phenyl isocyanate to give the title compound. ESMS [M+H]+m/z 494.4.

### Example 3

### Preparation of N-{[2-({[(2,6-dimethylphenyl)amino]carbonyl}amino)-4,5,6,7-tetrahydro-1-benzothien-3-yl]carbonyl}-L-valine

### a). Preparation of 2-amino-4,5,6,7-tetrahydro-1-benzothiophene-3-carboxylic acid (Example of Intermediate 3)

A suspension of ethyl 2-amino-4,5,6,7-tetrahydro-1-benzothiophene-3-carboxylate (10 g) in 100 mL 2.5 N sodium hydroxide (NaOH) solution (1:1 water/ethanol) was heated at 800° C. overnight. After the mixture was cooled and filtered to remove unreacted starting material, the solution was concentrated in vacuo. The residue was acidified to pH 3 by addition of 6N HCl and then filtered, resulting in a light brown solid (7.43 grams, ESMS [M+H]+m/z 198.3).

### b). Preparation of N-{[2-({[(2,6-dimethylphenyl)amino]carbonyl}amino)-4,5,6,7-tetrahydro-1-benzothien-3-yl]carbonyl}-L-valine

The title compound was prepared by the same procedure as in Example 1 except that L-Asp(tBu)-Wang resin was replaced with L-Val-Wang resin (obtained from Polymer Lab, 0.8 mmol/g) and 3-amino-2-naphthalenecarboxylic acid was replaced with 2-amino-4,5,6,7-tetrahydro-I-benzothiophene-3-carboxylic acid (obtained from 3a). to give the title compound. ESMS [M+H]+m/z 444.6

### Example 4

### Preparation of (2S)-cyclohexyl({[2-({[(2,6-dimethylphenyl)amino]carbonyl}amino)-1-benzothien-3-yl]carbonyl}amino)acetic acid

### a). Preparation of 2-amino-1-benzothiophene-3-carboxylic acid

Ethyl 2-amino-I-benzothiophene-3-carboxylate (obtained according to procedures described in Hallas, G.; Towns, A. D., Dyes and Pigments (1997), 35, 219-237) (10 g, 40.0 mmol) was suspended in ethanol (100 mL), and heated to reflux. A solution of potassium hydroxide (KOH, 8.4 g) in water (100 mL) was added over the period of 10 minutes. The reaction mixture was refluxed for another 10 minutes, cooled to room temperature, and filtered. The collected solid was washed with water to pH neutral to give the title compound as a brown solid (1.0 g, 13.0% yield). ESMS [M+H]+m/z 194.2.

### b). Preparation of (2S)-cyclohexyl({[2-({[(2,6-dimethylphenyl)amino]carbonyl}amino)-1-benzothien-3-yl]carbonyl}amino)ethanoic acid

The title compound was prepared by the same procedure as in Example 2 except that 3-amino-2-naphthalenecarboxylic acid was replaced by 2-amino-1-benzothiophene-3-carboxylic acid (obtained from 4a) and 2-chloro-6-methyl phenylisocyanate was replaced with 2,6-dimethyl phenylisocyanate to give the title compound. ESMS [M+H]+m/z 480.6.

### Example 5

### Preparation of {[3-({[(2-chloro-6-methylphenyl)amino]carbonyl}amino)-2-naphthoyl]amino}(piperidin-3-yl)acetic acid trifluoroacetate (Method B)

### a). Preparation of 3-({[(2-chloro-6-methylphenyl)amino]carbonyl}amino)-2-naphthoic acid (Example of Intermediate 6)

3-Amino-2-naphthoic acid (0.56 g, 85% technical grade, 2.5 mmol) was dissolved in 8 mL DMSO and treated with 2-chloro-6-methylphenylisocyanate (440 uL, 3.2 mmol) then shaken at room temperature for 20 hours. The reaction mixture was diluted with 25 mL H₂O and the resulting tan solid precipitate filtered, rinsed with H₂O (3×10 mL), dioxane (1×3 mL), and acetonitrile (1×3 mL) and dried in vacuo. Yield=0.58 g (64%). ESMS [M+H]+m/z 355.2.

### b). Preparation of {[3-({[(2-chloro-6-methylphenyl)amino]carbonyl}amino)-2-naphthoyl]amino}(piperidin-3-yl)acetic acid trifluoroacetate

N-Fmoc-(1-Boc-piperidin-3-yl)-D,L-glycine-Wang resin was prepared as described in Example 2a. Fmoc deprotection was achieved as in Example 2b. Approximately 90 mg (90 umol) of the resulting (1-Boc-piperidin-3-yl)-D,L-glycine-Wang resin was loaded into an IRORI Minikan and treated with a solution of 5 eq. (0.159 g, 450 umol) 3-({[(2-chloro-6-methylphenyl)amino]carbonyl}amino)-2-naphthoic acid (obtained as in example 5a) and 6 eq. HOAt (1-Hydroxy-7-aza-benzotriazole, 0.073 g, 540 umol) dissolved in 3 mL NMP (N-methylpyrrolidinone). To this mixture was added 7 eq. DIC (1,3-diisopropylcarbodiimide, 100uL, 630 umol) and the resulting reaction mixture shaken at room temperature for 20 hours. The solution was removed and the resin-filled Minikan washed with NMP (1×5 mL), CH₂Cl₂ (1×5 mL), MeOH (2×5 mL), CH₂Cl₂ (1×5 mL), MeOH (1×5 mL), and CH₂Cl₂ (3×5 mL). The resin was then cleaved in 1:1 TFA/CH₂Cl₂ (2 mL) for 2 hours. The cleavage solution was removed and the Minikan washed with CH₂Cl₂ (2×2 mL). Cleavage solution and washes were combined, dried, taken up in 0.5 mL DMSO and subjected to HPLC purification to provide the title compound as a tan solid film. ESMS [M+H]+m/z 495.6.

### Example 6

### Preparation of 3-({[(2-chloro-6-methylphenyl)amino]carbonyl}amino)-N-[(3-methylisoxazol-5-yl)methyl]-2-naphthamide (Method C)

3-({[(2-chloro-6-methylphenyl)amino]carbonyl}amino)-2-naphthoic acid (from Example 5a) (0.044 g, 125 umol) was dissolved in 0.25 mL DMSO and added to a mixture of (3-methyl-isoxazol-5-yl)-methylamine HCl (0.020 g, 134 umol) and DIEA (diisopropylethylamine, 87 uL, 500 umol) dissolved in 0.25 mL DMSO. DIC (1,3-diisopropylcarbodiimide, 60 uL, 375 umol) was added and the reaction mixture shaken at room temperature for 20 hours. The reaction mixture was directly subjected to HPLC purification to provide the title compound as a white solid. Yield=0.019 g (34%). ESMS [M+H]⁺ m/z 449.2.

### Example 7

### Preparation of (2S)-cyclohexyl{[(3-{[(2-methylphenyl)acetyl]amino}-2-naphthalenyl)carbonyl]amino}ethanoic acid (Method D)

### a). Preparation of Resin Bound (2S)-{[(3-amino-2-naphthalenyl)carbonyl]amino}(cyclohexyl)ethanoic acid on Wang Resin

The title compound was prepared by the same procedure as in Example 1b except that L-Asp(tBu)-Wang resin was replaced with L-CHG-Wang resin in a polypropylene tube (resin prepared as in 2b) to give the title compound. The resin was drained and washed with NMP until the yellow color is gone. The resin was then washed with DCM (3×100 mL), methanol (3×100 mL), DCM (3×100 mL), acetonitrile (3×100 mL), and DCM (3×100 mL) and dried in vacuo. A small portion of resin was taken out, cleaved by 1:1 TFA: DCM for 30 min at room temperature. LC-MS showed 100% formation of desired coupling intermediate product.

### b). Preparation of (2S)-cyclohexyl{[(3-{[(2-methylphenyl)acetyl]amino}-2-naphthalenyl)carbonyl]amino}ethanoic acid

To the resin from 7a (100 mg) in 1 mL pyridine was added a solution of (2-methylphenyl)acetyl chloride (25 mg, 2 eq) in a minimal amount of DCM for transfer (0.5 mL). After 4 hours at room temperature, the resin was washed and the reaction progress checked by LCMS. The resin was retreated under these conditions, until the reaction was complete by LCMS. The resin was drained, and then washed with DCM (3×5 mL), acetonitrile (3×5 mL), DCM (3×5 mL), acetonitrile (3×5 mL), and DCM (3×5 mL), and then dried in vacuo. The resin was then cleaved in 1:1 TFA: DCM for 30 minutes. The crude product solution was concentrated in vacuo, taken up by DMSO (0.6 mL), purified by HPLC, and dried in vacuo to give the title compound as a white solid (16.4 mg). ESMS [M+H]+m/z 459.6.

By similar methods, the following compounds were prepared as shown, with characterization data given in Table 1.

### Example 8

N-[3-({[(2,6-dimethylphenyl)amino]carbonyl}amino)-2-naphthoyl]-L-aspartic acid. This compound was prepared as described in Example 1 except that 2-methylphenylisocyanate was substituted for 2,6-dimethylphenylisocyanate.

### Example 9

N-[3-({[(2-chlorophenyl)amino]carbonyl}amino)-2-naphthoyl]-L-aspartic acid. This compound was prepared as described in Example 1 except that 2-chlorophenylisocyanate was substituted for 2,6-dimethylphenylisocyanate.

### Example 10

N-[3-({[(2-methylphenyl)amino]carbonyl}amino)-2-naphthoyl]glycine. This compound was prepared as described in Example 1 except that 2-methylphenylisocyanate was substituted for 2,6-dimethylphenylisocyanate, and Fmoc-glycine Wang resin was substituted for Fmoc-L-Aspartic acid(Asp)(tBu)-Wang resin.

### Example 11

N-[3-({[(2,6-dimethylphenyl)amino]carbonyl}amino)-2-naphthoyl]glycine. This compound was prepared as described in Example 1 except Fmoc-glycine Wang resin was substituted for Fmoc-L-Asp(tBu)-Wang resin.

### Example 12

N-[3-({[(2-chlorophenyl)amino]carbonyl}amino)-2-naphthoyl]glycine. This compound was prepared as described in Example 1 except that 2-chlorophenylisocyanate was substituted for 2,6-dimethylphenylisocyanate, and Fmoc-glycine Wang resin was substituted for Fmoc-L-Asp(tBu)-Wang resin.

### Example 13

N-[3-({[(2-methylphenyl)amino]carbonyl}amino)-2-naphthoyl]-L-alanine. This compound was prepared as described in Example 1 except that 2-methylphenylisocyanate was substituted for 2,6-dimethylphenylisocyanate, and Fmoc-L-alanine Wang resin was substituted for Fmoc-L-Asp(tBu)-Wang resin.

### Example 14

N-[3-({[(2-methylphenyl)amino]carbonyl}amino)-2-naphthoyl]-L-threonine. This compound was prepared as described in Example 1 except that 2-methylphenylisocyanate was substituted for 2,6-dimethylphenylisocyanate, and Fmoc-L-threonine(tBu)-Wang resin was substituted for Fmoc-L-Asp(tBu)-Wang resin.

### Example 15

N-[3-({[(2-methylphenyl)amino]carbonyl}amino)-2-naphthoyl]-L-isoleucine. This compound was prepared as described in Example 1 except that 2-methylphenylisocyanate was substituted for 2,6-dimethylphenylisocyanate, and Fmoc-L-isoleucine-Wang resin was substituted for Fmoc-L-Asp(tBu)-Wang resin.

### Example 16

N-[3-({[(2-methylphenyl)amino]carbonyl}amino)-2-naphthoyl]-L-leucine. This compound was prepared as described in Example 1 except that 2-methylphenylisocyanate was substituted for 2,6-dimethylphenylisocyanate, and Fmoc-L-leucine-Wang resin was substituted for Fmoc-L-Asp(tBu)-Wang resin.

### Example 17

N-[3-({[(2-methylphenyl)amino]carbonyl}amino)-2-naphthoyl]-L-asparagine. This compound was prepared as described in Example 1 except that 2-methylphenylisocyanate was substituted for 2,6-dimethylphenylisocyanate, and Fmoc-L-asparagine(Trityl(Trt))-Wang resin was substituted for Fmoc-L-Asp(tBu)-Wang resin.

### Example 18

N-[3-({[(2,6-dimethylphenyl)amino]carbonyl}amino)-2-naphthoyl]-L-alanine. This compound was prepared as described in Example 1 except that Fmoc-L-alanine Wang resin was substituted for Fmoc-L-Asp(tBu)-Wang resin.

### Example 19

N-[3-({[(2,6-dimethylphenyl)amino]carbonyl}amino)-2-naphthoyl]-L-serine. This compound was prepared as described in Example 1 except that Fmoc-L-serine(tBu)-Wang resin was substituted for Fmoc-L-Asp(tBu)-Wang resin.

### Example 20

1-[3-({[(2,6-dimethylphenyl)amino]carbonyl}amino)-2-naphthoyl]-L-proline. This compound was prepared as described in Example 1 except that Fmoc-L-proline-Wang resin was substituted for Fmoc-L-Asp(tBu)-Wang resin.

### Example 21

N-[3-({[(2,6-dimethylphenyl)amino]carbonyl}amino)-2-naphthoyl]-L-valine. This compound was prepared as described in Example 1 except that Fmoc-L-valine-Wang resin was substituted for Fmoc-L-Asp(tBu)-Wang resin.

### Example 22

N-[3-({[(2,6-dimethylphenyl)amino]carbonyl}amino)-2-naphthoyl]-L-threonine. This compound was prepared as described in Example 1 except that Fmoc-L-threonine(tBu)-Wang resin was substituted for Fmoc-L-Asp(tBu)-Wang resin.

### Example 23

N-[3-({[(2,6-dimethylphenyl)amino]carbonyl}amino)-2-naphthoyl]-L-isoleucine. This compound was prepared as described in Example 1 except that Fmoc-L-isoleucine-Wang resin was substituted for Fmoc-L-Asp(tBu)-Wang resin.

### Example 24

N-[3-({[(2,6-dimethylphenyl)amino]carbonyl}amino)-2-naphthoyl]-L-leucine. This compound was prepared as described in Example 1 except that Fmoc-L-leucine-Wang resin was substituted for Fmoc-L-Asp(tBu)-Wang resin.

### Example 25

N-[3-({[(2,6-dimethylphenyl)amino]carbonyl}amino)-2-naphthoyl]-L-asparagine. This compound was prepared as described in Example 1 except that Fmoc-L-asparagine(Trt)-Wang resin was substituted for Fmoc-L-Asp(tBu)-Wang resin.

### Example 26

N-[3-({[(2,6-dimethylphenyl)amino]carbonyl}amino)-2-naphthoyl]-L-glutamine. This compound was prepared as described in Example 1 except that Fmoc-L-glutamine(Trt)-Wang resin was substituted for Fmoc-L-Asp(tBu)-Wang resin.

### Example 27

N-[3-({[(2-chlorophenyl)amino]carbonyl}amino)-2-naphthoyl]-L-alanine. This compound was prepared as described in Example 1 except that 2-chlorophenylisocyanate was substituted for 2,6-dimethylphenylisocyanate, and Fmoc-L-alanine-Wang resin was substituted for Fmoc-L-Asp(tBu)-Wang resin.

### Example 28

N-[3-({[(2-chlorophenyl)amino]carbonyl}amino)-2-naphthoyl]-L-serine. This compound was prepared as described in Example 1 except that 2-chlorophenylisocyanate was substituted for 2,6-dimethylphenylisocyanate, and Fmoc-L-serine(tBu)-Wang resin was substituted for Fmoc-L-Asp(tBu)-Wang resin.

### Example 29

N-[3-({[(2-chlorophenyl)amino]carbonyl}amino)-2-naphthoyl]-L-threonine. This compound was prepared as described in Example 1 except that 2-chlorophenylisocyanate was substituted for 2,6-dimethylphenylisocyanate, and Fmoc-L-threonine(tBu)-Wang resin was substituted for Fmoc-L-Asp(tBu)-Wang resin.

### Example 30

N-[3-({[(2-chlorophenyl)amino]carbonyl}amino)-2-naphthoyl]-L-isoleucine. This compound was prepared as described in Example 1 except that 2-chlorophenylisocyanate was substituted for 2,6-dimethylphenylisocyanate, and Fmoc-L-isoleucine-Wang resin was substituted for Fmoc-L-Asp(tBu)-Wang resin.

### Example 31

N-[3-({[(2-chlorophenyl)amino]carbonyl}amino)-2-naphthoyl]-L-asparagine. This compound was prepared as described in Example 1 except that 2-chlorophenylisocyanate was substituted for 2,6-dimethylphenylisocyanate, and Fmoc-L-asparagine(Trt)-Wang resin was substituted for Fmoc-L-Asp(tBu)-Wang resin.

### Example 32

N-[3-({[(2-chlorophenyl)amino]carbonyl}amino)-2-naphthoyl]-L-glutamine. This compound was prepared as described in Example 1 except that 2-chlorophenylisocyanate was substituted for 2,6-dimethylphenylisocyanate, and Fmoc-L-glutamine(Trt)-Wang resin was substituted for Fmoc-L-Asp(tBu)-Wang resin.

### Example 33

N-[3-({[(2-chloro-6-methylphenyl)amino]carbonyl}amino)-2-naphthoyl]-L-alanine. This compound was prepared as described in Example 1 except that 2-chloro-6-methylphenylisocyanate was substituted for 2,6-dimethylphenylisocyanate, and Fmoc-L-alanine-Wang resin was substituted for Fmoc-L-Asp(tBu)-Wang resin.

### Example 34

N-[3-({[(2-chloro-6-methylphenyl)amino]carbonyl}amino)-2-naphthoyl]-L-serine. This compound was prepared as described in Example 1 except that 2-chloro-6-methylphenylisocyanate was substituted for 2,6-dimethylphenylisocyanate, and Fmoc-L-serine(tBu)-Wang resin was substituted for Fmoc-L-Asp(tBu)-Wang resin.

### Example 35

1-[3-({[(2-chloro-6-methylphenyl)amino]carbonyl}amino)-2-naphthoyl]-L-proline. This compound was prepared as described in Example 1 except that 2-chloro-6-methylphenylisocyanate was substituted for 2,6-dimethylphenylisocyanate, and Fmoc-L-proline-Wang resin was substituted for Fmoc-L-Asp(tBu)-Wang resin.

### Example 36

N-[3-({[(2-chloro-6-methylphenyl)amino]carbonyl}amino)-2-naphthoyl]-L-valine. This compound was prepared as described in Example 1 except that 2-chloro-6-methylphenylisocyanate was substituted for 2,6-dimethylphenylisocyanate, and Fmoc-L-valine-Wang resin was substituted for Fmoc-L-Asp(tBu)-Wang resin.

### Example 37

N-[3-({[(2-chloro-6-methylphenyl)amino]carbonyl}amino)-2-naphthoyl]-L-threonine. This compound was prepared as described in Example 1 except that 2-chloro-6-methylphenylisocyanate was substituted for 2,6-dimethylphenylisocyanate, and Fmoc-L-threonine(tBu)-Wang resin was substituted for Fmoc-L-Asp(tBu)-Wang resin.

### Example 38

N-[3-({[(2-chloro-6-methylphenyl)amino]carbonyl}amino)-2-naphthoyl]-L-isoleucine. This compound was prepared as described in Example 1 except that 2-chloro-6-methylphenylisocyanate was substituted for 2,6-dimethylphenylisocyanate, and Fmoc-L-isoleucine-Wang resin was substituted for Fmoc-L-Asp(tBu)-Wang resin.

### Example 39

N-[3-({[(2-chloro-6-methylphenyl)amino]carbonyl}amino)-2-naphthoyl]-L-leucine. This compound was prepared as described in Example 1 except that 2-chloro-6-methylphenylisocyanate was substituted for 2,6-dimethylphenylisocyanate, and Fmoc-L-leucine-Wang resin was substituted for Fmoc-L-Asp(tBu)-Wang resin.

### Example 40

N-[3-({[(2-chloro-6-methylphenyl)amino]carbonyl}amino)-2-naphthoyl]-L-asparagine. This compound was prepared as described in Example 1 except that 2-chloro-6-methylphenylisocyanate was substituted for 2,6-dimethylphenylisocyanate, and Fmoc-L-asparagine(Trt)-Wang resin was substituted for Fmoc-L-Asp(tBu)-Wang resin.

### Example 41

N-[3-({[(2-chloro-6-methylphenyl)amino]carbonyl}amino)-2-naphthoyl]-L-glutamine. This compound was prepared as described in Example 1 except that 2-chloro-6-methylphenylisocyanate was substituted for 2,6-dimethylphenylisocyanate, and Fmoc-L-glutamine(Trt)-Wang resin was substituted for Fmoc-L-Asp(tBu)-Wang resin.

### Example 42

N-[3-({[(2-methylphenyl)amino]carbonyl}amino)-2-naphthoyl]-L-glutamic acid. This compound was prepared as described in Example 1 except that 2-methylphenylisocyanate was substituted for 2,6-dimethylphenylisocyanate, and Fmoc-L-glutamic acid(tBu)-Wang resin was substituted for Fmoc-L-Asp(tBu)-Wang resin.

### Example 43

N-[3-({[(2-methylphenyl)amino]carbonyl}amino)-2-naphthoyl]-L-methionine. This compound was prepared as described in Example 1 except that 2-methylphenylisocyanate was substituted for 2,6-dimethylphenylisocyanate, and Fmoc-L-methionine-Wang resin was substituted for Fmoc-L-Asp(tBu)-Wang resin.

### Example 44

N-[3-({[(2-methylphenyl)amino]carbonyl}amino)-2-naphthoyl]-L-histidine trifluoroacetate. This compound was prepared as described in Example 1 except that 2-methylphenylisocyanate was substituted for 2,6-dimethylphenylisocyanate, and Fmoc-L-histidine(Trt)-Wang resin was substituted for Fmoc-L-Asp(tBu)-Wang resin.

### Example 45

N-[3-({[(2-methylphenyl)amino]carbonyl}amino)-2-naphthoyl]-L-phenylalanine. This compound was prepared as described in Example 1 except that 2-methylphenylisocyanate was substituted for 2,6-dimethylphenylisocyanate, and Fmoc-L-phenylalanine-Wang resin was substituted for Fmoc-L-Asp(tBu)-Wang resin.

### Example 46

N-[3-({[(2-methylphenyl)amino]carbonyl}amino)-2-naphthoyl]-L-tryptophan. This compound was prepared as described in Example 1 except that 2-methylphenylisocyanate was substituted for 2,6-dimethylphenylisocyanate, and Fmoc-L-tryptophan(Boc)-Wang resin was substituted for Fmoc-L-Asp(tBu)-Wang resin.

### Example 47

N-[3-({[(2,6-dimethylphenyl)amino]carbonyl}amino)-2-naphthoyl]-L-lysine trifluoroacetate. This compound was prepared as described in Example I except Fmoc-L-lysine-Wang resin was substituted for Fmoc-L-Asp(tBu)-Wang resin.

### Example 48

N-[3-({[(2,6-dimethylphenyl)amino]carbonyl}amino)-2-naphthoyl]-L-glutamic acid. This compound was prepared as described in Example 1 except Fmoc-L-glutamic acid(tBu)-Wang resin was substituted for Fmoc-L-Asp(tBu)-Wang resin.

### Example 49

N-[3-({[(2,6-dimethylphenyl)amino]carbonyl}amino)-2-naphthoyl]-L-methionine. This compound was prepared as described in Example 1 except Fmoc-L-methionine-Wang resin was substituted for Fmoc-L-Asp(tBu)-Wang resin.

### Example 50

N-[3-({[(2,6-dimethylphenyl)amino]carbonyl}amino)-2-naphthoyl]-L-histidine trifluoroacetate. This compound was prepared as described in Example 1 except Fmoc-L-histidine(Trt)-Wang resin was substituted for Fmoc-L-Asp(tBu)-Wang resin.

### Example 51

N-[3-({[(2,6-dimethylphenyl)amino]carbonyl}amino)-2-naphthoyl]-L-phenylalanine. This compound was prepared as described in Example 1 except Fmoc-L-phenylalanine-Wang resin was substituted for Fmoc-L-Asp(tBu)-Wang resin.

### Example 52

N-[3-({[(2,6-dimethylphenyl)amino]carbonyl}amino)-2-naphthoyl]-L-arginine. This compound was prepared as described in Example 1 except Fmoc-L-arginine(2,2,4,6,7-pentamethyldihydrobenzofuran-5-sulfonyl(Pbf))-Wang resin was substituted for Fmoc-L-Asp(tBu)-Wang resin.

### Example 53

N-[3-({[(2,6-dimethylphenyl)amino]carbonyl}amino)-2-naphthoyl]-L-tyrosine. This compound was prepared as described in Example 1 except Fmoc-L-tyrosine(tBu)-Wang resin was substituted for Fmoc-L-Asp(tBu)-Wang resin.

### Example 54

N-[3-({[(2,6-dimethylphenyl)amino]carbonyl}amino)-2-naphthoyl]-L-tryptophan trifluoroacetate. This compound was prepared as described in Example 1 except Fmoc-L-tryptophan(Boc)-Wang resin was substituted for Fmoc-L-Asp(tBu)-Wang resin.

### Example 55

N-[3-({[(2-chlorophenyl)amino]carbonyl}amino)-2-naphthoyl]-L-glutamic acid. This compound was prepared as described in Example 1 except that 2-chlorophenylisocyanate was substituted for 2,6-dimethylphenylisocyanate, and Fmoc-L-glutamic acid(tBu)-Wang resin was substituted for Fmoc-L-Asp(tBu)-Wang resin.

### Example 56

N-[3-({[(2-chlorophenyl)amino]carbonyl}amino)-2-naphthoyl]-L-histidine trifluoroacetate. This compound was prepared as described in Example 1 except that 2-chlorophenylisocyanate was substituted for 2,6-dimethylphenylisocyanate, and Fmoc-L-histidine(Trt)-Wang resin was substituted for Fmoc-L-Asp(tBu)-Wang resin.

### Example 57

N-[3-({[(2-chlorophenyl)amino]carbonyl}amino)-2-naphthoyl]-L-phenylalanine. This compound was prepared as described in Example 1 except that 2-chlorophenylisocyanate was substituted for 2,6-dimethylphenylisocyanate, and Fmoc-L-phenylalanine-Wang resin was substituted for Fmoc-L-Asp(tBu)-Wang resin.

### Example 58

N-[3-({[(2-chlorophenyl)amino]carbonyl}amino)-2-naphthoyl]-L-tryptophan trifluoroacetate. This compound was prepared as described in Example 1 except that 2-chlorophenylisocyanate was substituted for 2,6-dimethylphenylisocyanate, and Fmoc-L-tryptophan(Boc)-Wang resin was substituted for Fmoc-L-Asp(tBu)-Wang resin.

### Example 59

N-[3-({[(2-chloro-6-methylphenyl)amino]carbonyl}amino)-2-naphthoyl]-L-lysine trifluoroacetate. This compound was prepared as described in Example 1 except that 2-chloro-6-methylphenylisocyanate was substituted for 2,6-dimethylphenylisocyanate, and Fmoc-L-lysine(Boc)-Wang resin was substituted for Fmoc-L-Asp(tBu)-Wang resin.

### Example 60

N-[3-({[(2-chloro-6-methylphenyl)amino]carbonyl}amino)-2-naphthoyl]-L-methionine. This compound was prepared as described in Example 1 except that 2-chloro-6-methylphenylisocyanate was substituted for 2,6-dimethylphenylisocyanate, and Fmoc-L-methionine-Wang resin was substituted for Fmoc-L-Asp(tBu)-Wang resin.

### Example 61

N-[3-({[(2-chloro-6-methylphenyl)amino]carbonyl}amino)-2-naphthoyl]-L-histidine trifluoroacetate. This compound was prepared as described in Example 1 except that 2-chloro-6-methylphenylisocyanate was substituted for 2,6-dimethylphenylisocyanate, and Fmoc-L-histidine(Trt)-Wang resin was substituted for Fmoc-L-Asp(tBu)-Wang resin.

### Example 62

N-[3-({[(2-chloro-6-methylphenyl)amino]carbonyl}amino)-2-naphthoyl]-L-phenylalanine. This compound was prepared as described in Example 1 except that 2-chloro-6-methylphenylisocyanate was substituted for 2,6-dimethylphenylisocyanate, and Fmoc-L-phenylalanine-Wang resin was substituted for Fmoc-L-Asp(tBu)-Wang resin.

### Example 63

N-[3-({[(2-chloro-6-methylphenyl)amino]carbonyl}amino)-2-naphthoyl]-L-arginine. This compound was prepared as described in Example 1 except that 2-chloro-6-methylphenylisocyanate was substituted for 2,6-dimethylphenylisocyanate, and Fmoc-L-arginine(Pbf)-Wang resin was substituted for Fmoc-L-Asp(tBu)-Wang resin.

### Example 64

N-[3-({[(2-chloro-6-methylphenyl)amino]carbonyl}amino)-2-naphthoyl]-L-tyrosine. This compound was prepared as described in Example 1 except that 2-chloro-6-methylphenylisocyanate was substituted for 2,6-dimethylphenylisocyanate, and Fmoc-L-tyrosine(tBu)-Wang resin was substituted for Fmoc-L-Asp(tBu)-Wang resin.

### Example 65

N-[3-({[(2-chloro-6-methylphenyl)amino]carbonyl}amino)-2-naphthoyl]-L-tryptophan trifluoroacetate. This compound was prepared as described in Example 1 except that 2-chloro-6-methylphenylisocyanate was substituted for 2,6-dimethylphenylisocyanate, and Fmoc-L-tryptophan(Boc)-Wang resin was substituted for Fmoc-L-Asp(tBu)-Wang resin.

### Example 66

N-[2-({[(2-chloro-6-methylphenyl)amino]carbonyl}amino)-4,5-difluorobenzoyl]-L-aspartic acid. This compound was prepared as described in Example 1 except that 2-chloro-6-methylphenylisocyanate was substituted for 2,6-dimethylphenylisocyanate, and 2-amino-4,5-difluorobenzoic acid was substituted for 3-amino-2-naphthalenecarboxylic acid.

### Example 67

N-[2-({[(2-chloro-6-methylphenyl)amino]carbonyl}amino)-4,5-dimethoxybenzoyl]-L-aspartic acid. This compound was prepared as described in Example 1 except that 2-chloro-6-methylphenylisocyanate was substituted for 2,6-dimethylphenylisocyanate, and 2-amino-4,5-dimethoxybenzoic acid was substituted for 3-amino-2-naphthalenecarboxylic acid.

### Example 68

N-[2-({[(2-chloro-6-methylphenyl)amino]carbonyl}amino)-4,5-difluorobenzoyl]-L-leucine. This compound was prepared as described in Example 1 except that 2-chloro-6-methylphenylisocyanate was substituted for 2,6-dimethylphenylisocyanate, 2-amino-4,5-difluorobenzoic acid was substituted for 3-amino-2-naphthalenecarboxylic acid, and Fmoc-L-leucine-Wang resin was substituted for Fmoc-L-Asp(tBu)-Wang resin.

### Example 69

N-[2-({[(2-chloro-6-methylphenyl)amino]carbonyl}amino)-4,5-dimethoxybenzoyl]-L-leucine. This compound was prepared as described in Example 1 except that 2-chloro-6-methylphenylisocyanate was substituted for 2,6-dimethylphenylisocyanate, 2-amino-4,5-dimethoxybenzoic acid was substituted for 3-amino-2-naphthalenecarboxylic acid, and Fmoc-L-leucine-Wang resin was substituted for Fmoc-L-Asp(tBu)-Wang resin.

### Example 70

N-[2-({[(2-chloro-6-methylphenyl)amino]carbonyl}amino)-4,5-difluorobenzoyl]-L-isoleucine. This compound was prepared as described in Example 1 except that 2-chloro-6-methylphenylisocyanate was substituted for 2,6-dimethylphenylisocyanate, 2-amino-4,5-difluorobenzoic acid was substituted for 3-amino-2-naphthalenecarboxylic acid, and Fmoc-L-isoleucine-Wang resin was substituted for Fmoc-L-Asp(tBu)-Wang resin.

### Example 71

N-[2-({[(2-chloro-6-methylphenyl)amino]carbonyl}amino)-4,5-dimethoxybenzoyl]-L-isoleucine. This compound was prepared as described in Example 1 except that 2-chloro-6-methylphenylisocyanate was substituted for 2,6-dimethylphenylisocyanate, 2-amino-4,5-dimethoxybenzoic acid was substituted for 3-amino-2-naphthalenecarboxylic acid, and Fmoc-L-isoleucine-Wang resin was substituted for Fmoc-L-Asp(tBu)-Wang resin.

### Example 72

N-[2-({[(2-chloro-6-methylphenyl)amino]carbonyl}amino)-4,5-difluorobenzoyl]-L-phenylalanine. This compound was prepared as described in Example 1 except that 2-chloro-6-methylphenylisocyanate was substituted for 2,6-dimethylphenylisocyanate, 2-amino-4,5-difluorobenzoic acid was substituted for 3-amino-2-naphthalenecarboxylic acid, and Fmoc-L-phenylalanine-Wang resin was substituted for Fmoc-L-Asp(tBu)-Wang resin.

### Example 73

N-[2-({[(2-chloro-6-methylphenyl)amino]carbonyl}amino)-4,5-dimethoxybenzoyl]-L-phenylalanine. This compound was prepared as described in Example 1 except that 2-chloro-6-methylphenylisocyanate was substituted for 2,6-dimethylphenylisocyanate, 2-amino-4,5-dimethoxybenzoic acid was substituted for 3-amino-2-naphthalenecarboxylic acid, and Fmoc-L-phenylalanine-Wang resin was substituted for Fmoc-L-Asp(tBu)-Wang resin.

### Example 74

N-[2-({[(2-chloro-6-methylphenyl)amino]carbonyl}amino)-4,5-difluorobenzoyl]-L-tryptophan trifluoroacetate. This compound was prepared as described in Example 1 except that 2-chloro-6-methylphenylisocyanate was substituted for 2,6-dimethylphenylisocyanate, 2-amino-4,5-difluorobenzoic acid was substituted for 3-amino-2-naphthalenecarboxylic acid, and Fmoc-L-tryptophan(Boc)-Wang resin was substituted for Fmoc-L-Asp(tBu)-Wang resin.

### Example 75

N-[2-({[(2-chloro-6-methylphenyl)amino]carbonyl}amino)-4,5-dimethoxybenzoyl]-L-tryptophan trifluoroacetate. This compound was prepared as described in Example 1 except that 2-chloro-6-methylphenylisocyanate was substituted for 2,6-dimethylphenylisocyanate, 2-amino-4,5-dimethoxybenzoic acid was substituted for 3-amino-2-naphthalenecarboxylic acid, and Fmoc-L-tryptophan(Boc)-Wang resin was substituted for Fmoc-L-Asp(tBu)-Wang resin.

### Example 76

N-[2-({[(2-chlorophenyl)amino]carbonyl}amino)-4,5-difluorobenzoyl]-L-tryptophan trifluoroacetate. This compound was prepared as described in Example 1 except that 2-chlorophenylisocyanate was substituted for 2,6-dimethylphenylisocyanate, 2-amino-4,5-difluorobenzoic acid was substituted for 3-amino-2-naphthalenecarboxylic acid, and Fmoc-L-tryptophan(Boc)-Wang resin was substituted for Fmoc-L-Asp(tBu)-Wang resin.

### Example 77

N-[2-({[(2-chlorophenyl)amino]carbonyl}amino)-4,5-dimethoxybenzoyl]-L-tryptophan trifluoroacetate. This compound was prepared as described in Example 1 except that 2-chlorophenylisocyanate was substituted for 2,6-dimethylphenylisocyanate, 2-amino-4,5-dimethoxybenzoic acid was substituted for 3-amino-2-naphthalenecarboxylic acid, and Fmoc-L-tryptophan(Boc)-Wang resin was substituted for Fmoc-L-Asp(tBu)-Wang resin.

### Example 78

N-[2-({[(2,6-dimethylphenyl)amino]carbonyl}amino)-4,5-difluorobenzoyl]-L-aspartic acid. This compound was prepared as described in Example 1 except that 2-amino-4,5-difluorobenzoic acid was substituted for 3-amino-2-naphthalenecarboxylic acid.

### Example 79

N-[2-({[(2,6-dimethylphenyl)amino]carbonyl}amino)-4,5-difluorobenzoyl]-L-leucine. This compound was prepared as described in Example 1 except that 2-amino-4,5-difluorobenzoic acid was substituted for 3-amino-2-naphthalenecarboxylic acid, and Fmoc-L-leucine-Wang resin was substituted for Fmoc-L-Asp(tBu)-Wang resin.

### Example 80

N-[2-({[(2,6-dimethylphenyl)amino]carbonyl}amino)-4,5-difluorobenzoyl]-L-isoleucine. This compound was prepared as described in Example 1 except that 2-amino-4,5-difluorobenzoic acid was substituted for 3-amino-2-naphthalenecarboxylic acid, and Fmoc-L-isoleucine-Wang resin was substituted for Fmoc-L-Asp(tBu)-Wang resin.

### Example 81

N-[2-({[(2,6-dimethylphenyl)amino]carbonyl}amino)-4,5-difluorobenzoyl]-L-phenylalanine. This compound was prepared as described in Example 1 except that 2-amino-4,5-difluorobenzoic acid was substituted for 3-amino-2-naphthalenecarboxylic acid, and Fmoc-L-phenylalanine-Wang resin was substituted for Fmoc-L-Asp(tBu)-Wang resin.

### Example 82

N-[2-({[(2,6-dimethylphenyl)amino]carbonyl}amino)-4,5-dimethoxybenzoyl]-L-tryptophan trifluoroacetate. This compound was prepared as described in Example 1 except that 2-amino-4,5-dimethoxybenzoic acid was substituted for 3-amino-2-naphthalenecarboxylic acid, and Fmoc-L-tryptophan(Boc)-Wang resin was substituted for Fmoc-L-Asp(tBu)-Wang resin.

### Example 83

N-[2-({[(2,6-diethylphenyl)amino]carbonyl}amino)benzoyl]-L-aspartic acid. This compound was prepared as described in Example 1 except that 2,6-diethylphenylisocyanate was substituted for 2,6-dimethylphenylisocyanate, and 2-aminobenzoic acid was substituted for 3-amino-2-naphthalenecarboxylic acid.

### Example 84

N-[3-({[(2-chloro-6-methylphenyl)amino]carbonyl}amino)-2-naphthoyl]-L-aspartic acid. This compound was prepared as described in Example 1 except that 2-chloro-6-methylphenylisocyanate was substituted for 2,6-dimethylphenylisocyanate.

### Example 85

N-[3-({[(2-chloro-6-methylphenyl)amino]carbonyl}amino)-2-naphthoyl]glycine. This compound was prepared as described in Example 1 except that 2-chloro-6-methylphenylisocyanate was substituted for 2,6-dimethylphenylisocyanate, and Fmoc-glycine-Wang resin was substituted for Fmoc-L-Asp(tBu)-Wang resin.

### Example 86

N-[3-({[(2-chloro-6-methylphenyl)amino]carbonyl}amino)-2-naphthoyl]-L-glutamic acid. This compound was prepared as described in Example 1 except that 2-chloro-6-methylphenylisocyanate was substituted for 2,6-dimethylphenylisocyanate, and Fmoc-glutamic acid(tBu)-Wang resin was substituted for Fmoc-L-Asp(tBu)-Wang resin.

### Example 87

N-[2-({[(2-chloro-6-methylphenyl)amino]carbonyl}amino)benzoyl]-L-aspartic acid. This compound was prepared as described in Example 1 except that 2-chloro-6-methylphenylisocyanate was substituted for 2,6-dimethylphenylisocyanate, and 2-aminobenzoic acid was substituted for 3-amino-2-naphthalenecarboxylic acid.

### Example 88

N-[4-chloro-2-({[(2-chloro-6-methylphenyl)amino]carbonyl}amino)benzoyl]-L-aspartic acid. This compound was prepared as described in Example 1 except that 2-chloro-6-methylphenylisocyanate was substituted for 2,6-dimethylphenylisocyanate, and 2-amino-4-chlorobenzoic acid was substituted for 3-amino-2-naphthalenecarboxylic acid.

### Example 89

N-[2-({[(2-chloro-6-methylphenyl)amino]carbonyl}amino)-5-iodobenzoyl]-L-aspartic acid. This compound was prepared as described in Example 1 except that 2-chloro-6-methylphenylisocyanate was substituted for 2,6-dimethylphenylisocyanate, and 2-amino-5-iodobenzoic acid was substituted for 3-amino-2-naphthalenecarboxylic acid.

### Example 90

N-[3-({[(2-bromophenyl)amino]carbonyl}amino)-2-naphthoyl]-L-aspartic acid. This compound was prepared as described in Example 1 except that 2-bromophenylisocyanate was substituted for 2,6-dimethylphenylisocyanate.

### Example 91

4-bromo-N-[3-({[(2-chloro-6-methylphenyl)amino]carbonyl}amino)-2-naphthoyl]-L-phenylalanine. This compound was prepared as described in Example 2 except that and Fmoc-L-4-bromophenylalanine was substituted for Fmoc-L-cyclohexylglycine.

### Example 92

(2S)-cyclohexyl{[3-({[(2,6-dimethylphenyl)amino]carbonyl}amino)-2-naphthoyl]amino}acetic acid. This compound was prepared as described in Example 2 except that 2,6-dimethylphenylisocyanate was substituted for 2-chloro-6-methylphenylisocyanate.

### Example 93

(2S)-cyclohexyl({[3-({[(2,6-diethylphenyl)amino]carbonyl}amino)-2-naphthalenyl]carbonyl}amino)ethanoic acid. This compound was prepared as described in Example 2 except that 2,6-diethylphenylisocyanate was substituted for 2-chloro-6-methylphenylisocyanate.

### Example 94

(2S)-cyclohexyl{[2-({[(2,6-dimethylphenyl)amino]carbonyl}amino)benzoyl]amino}acetic acid. This compound was prepared as described in Example 2 except that 2,6-dimethylphenylisocyanate was substituted for 2-chloro-6-methylphenylisocyanate, and 2-2-aminobenzoic acid was substituted for 3-amino-2-naphthalenecarboxylic acid.

### Example 95

{[3-({[(2-methylphenyl)amino]carbonyl}amino)-2-naphthoyl]amino}(phenyl)acetic acid. This compound was prepared as described in Example 2 except that 2-methylphenylisocyanate was substituted for 2-chloro-6-methylphenylisocyanate, and Fmoc-L-phenylglycine was substituted for Fmoc-L-cyclohexylglycine.

### Example 96

N-{[3-({[(2-methylphenyl)amino]carbonyl}amino)-2-naphthalenyl]carbonyl}-3-(2-thienyl)-L-alanine. This compound was prepared as described in Example 2 except that 2-methylphenylisocyanate was substituted for 2-chloro-6-methylphenylisocyanate, and Fmoc-L-2-thienylalanine was substituted for Fmoc-L-cyclohexylglycine.

### Example 97

{[3-({[(2,6-dimethylphenyl)amino]carbonyl}amino)-2-naphthoyl]amino}(phenyl)acetic acid. This compound was prepared as described in Example 2 except that 2,6-dimethylphenylisocyanate was substituted for 2-chloro-6-methylphenylisocyanate, and Fmoc-L-phenylglycine was substituted for Fmoc-L-cyclohexylglycine.

### Example 98

N-{[3-({[(2,6-dimethylphenyl)amino]carbonyl}amino)-2-naphthalenyl]carbonyl}-3-(2-thienyl)-L-alanine. This compound was prepared as described in Example 2 except that 2,6-dimethylphenylisocyanate was substituted for 2-chloro-6-methylphenylisocyanate, and Fmoc-L-2-thienylalanine was substituted for Fmoc-L-cyclohexylglycine.

### Example 99

3-cyclohexyl-N-[3-({[(2,6-dimethylphenyl)amino]carbonyl}amino)-2-naphthoyl]-L-alanine. This compound was prepared as described in Example 2 except that 2,6-dimethylphenylisocyanate was substituted for 2-chloro-6-methylphenylisocyanate, and Fmoc-L-cyclohexylalanine was substituted for Fmoc-L-cyclohexylglycine.

### Example 100

{[3-({[(2-chlorophenyl)amino]carbonyl}amino)-2-naphthoyl]amino}(phenyl)acetic acid. This compound was prepared as described in Example 2 except that 2-chlorophenylisocyanate was substituted for 2-chloro-6-methylphenylisocyanate, and Fmoc-L-phenylglycine was substituted for Fmoc-L-cyclohexylglycine.

### Example 101

N-{[3-({[(2-chlorophenyl)amino]carbonyl}amino)-2-naphthalenyl]carbonyl}-3-(2-thienyl)-L-alanine. This compound was prepared as described in Example 2 except that 2-chlorophenylisocyanate was substituted for 2-chloro-6-methylphenylisocyanate, and Fmoc-L-2-thienylalanine was substituted for Fmoc-L-cyclohexylglycine.

### Example 102

N-{[3-({[(2-chloro-6-methylphenyl)amino]carbonyl}amino)-2-naphthalenyl]carbonyl}-3-(2-thienyl)-L-alanine. This compound was prepared as described in Example 2 except that Fmoc-L-2-thienylalanine was substituted for Fmoc-L-cyclohexylglycine.

### Example 103

Phenyl({[3-({[(2,4,6-trimethylphenyl)amino]carbonyl}amino)-2-naphthalenyl]carbonyl}amino)acetic acid. This compound was prepared as described in Example 2 except that 2,4,6-trimethylphenylisocyanate was substituted for 2-chloro-6-methylphenylisocyanate, and Fmoc-D-phenylglycine was substituted for Fmoc-L-cyclohexylglycine.

### Example 104

{[3-({[(2-isopropyl-6-methylphenyl)amino]carbonyl}amino)-2-naphthoyl]amino}(phenyl)acetic acid. This compound was prepared as described in Example 2 except that 2-isopropyl-6-methylphenylisocyanate was substituted for 2-chloro-6-methylphenylisocyanate, and Fmoc-D-phenylglycine was substituted for Fmoc-L-cyclohexylglycine.

### Example 105

{[2-({[(2,6-dimethylphenyl)amino]carbonyl}amino)-4,5-dimethoxybenzoyl]amino}(phenyl)acetic acid. This compound was prepared as described in Example 2 except that 2,6-dimethylphenylisocyanate was substituted for 2-chloro-6-methylphenylisocyanate, 2-amino-4,5-dimethoxybenzoic acid was substituted for 3-amino-2-naphthalenecarboxylic acid, and Fmoc-D-phenylglycine was substituted for Fmoc-L-cyclohexylglycine.

### Example 106

[(2-{[(mesitylamino)carbonyl]amino}-4,5-dimethoxybenzoyl)amino](phenyl)acetic acid. This compound was prepared as described in Example 2 except that 2,4,6-trimethylphenylisocyanate was substituted for 2-chloro-6-methylphenylisocyanate, 2-amino-4,5-dimethoxybenzoic acid was substituted for 3-amino-2-naphthalenecarboxylic acid, and Fmoc-D-phenylglycine was substituted for Fmoc-L-cyclohexylglycine.

### Example 107

{[2-({[(2-chloro-6-methylphenyl)amino]carbonyl}amino)-4,5-dimethoxybenzoyl]amino}(phenyl)acetic acid. This compound was prepared as described in Example 2 except that 2-amino-4,5-dimethoxybenzoic acid was substituted for 3-amino-2-naphthalenecarboxylic acid, and Fmoc-D-phenylglycine was substituted for Fmoc-L-cyclohexylglycine.

### Example 108

(2R)-cyclohexyl[(3-{[(mesitylamino)carbonyl]amino}-2-naphthoyl)amino]acetic acid. This compound was prepared as described in Example 2 except that 2,4,6-trimethylphenylisocyanate was substituted for 2-chloro-6-methylphenylisocyanate, and Fmoc-D-cyclohexylglycine was substituted for Fmoc-L-cyclohexylglycine.

### Example 109

(2R)-cyclohexyl{[3-({[(2-isopropyl-6-methylphenyl)amino]carbonyl}amino)-2-naphthoyl]amino}acetic acid. This compound was prepared as described in Example 2 except that 2-isopropyl-6-methylphenylisocyanate was substituted for 2-chloro-6-methylphenylisocyanate, and Fmoc-D-cyclohexylglycine was substituted for Fmoc-L-cyclohexylglycine.

### Example 110

(2R)-cyclohexyl{[3-({[(2,6-dimethylphenyl)amino]carbonyl}amino)-2-naphthoyl]amino}acetic acid. This compound was prepared as described in Example 2 except that 2,6-dimethylphenylisocyanate was substituted for 2-chloro-6-methylphenylisocyanate, and Fmoc-D-cyclohexylglycine was substituted for Fmoc-L-cyclohexylglycine.

### Example 111

(2R)-{[3-({[(2-chloro-6-methylphenyl)amino]carbonyl}amino)-2-naphthoyl]amino}(cyclohexyl)acetic acid. This compound was prepared as described in Example 2 except that Fmoc-D-cyclohexylglycine was substituted for Fmoc-L-cyclohexylglycine.

### Example 112

(2S)-{[2-({[(2-chloro-6-methylphenyl)amino]carbonyl}amino)-4,5-difluorobenzoyl]amino}(cyclohexyl)acetic acid. This compound was prepared as described in Example 2 except that 2-amino-4,5-difluorobenzoic acid was substituted for 3-amino-2-naphthalenecarboxylic acid.

### Example 113

(2S)-{[2-({[(2-chloro-6-methylphenyl)amino]carbonyl}amino)-4,5-dimethoxybenzoyl]amino}(cyclohexyl)acetic acid. This compound was prepared as described in Example 2 except that 2-amino-4,5-dimethoxybenzoic acid was substituted for 3-amino-2-naphthalenecarboxylic acid.

### Example 114

N-[2-({[(2-chloro-6-methylphenyl)amino]carbonyl}amino)-4,5-difluorobenzoyl]-3-cyclohexyl-L-alanine. This compound was prepared as described in Example 2 except that 2-amino-4,5-difluorobenzoic acid was substituted for 3-amino-2-naphthalenecarboxylic acid, and Fmoc-L-cyclohexylalanine was substituted for Fmoc-L-cyclohexylglycine.

### Example 115

N-[2-({[(2-chloro-6-methylphenyl)amino]carbonyl}amino)-4,5-dimethoxybenzoyl]-3-cyclohexyl-L-alanine. This compound was prepared as described in Example 2 except that 2-amino-4,5-dimethoxybenzoic acid was substituted for 3-amino-2-naphthalenecarboxylic acid, and Fmoc-L-cyclohexylalanine was substituted for Fmoc-L-cyclohexylglycine.

### Example 116

(2S)-cyclohexyl{[2-({[(2,6-dimethylphenyl)amino]carbonyl}amino)-4,5-difluorobenzoyl]amino}acetic acid. This compound was prepared as described in Example 2 except that 2,6-dimethylphenylisocyanate was substituted for 2-chloro-6-methylphenylisocyanate, and 2-amino-4,5-difluorobenzoic acid was substituted for 3-amino-2-naphthalenecarboxylic acid.

### Example 117

(2S)-cyclohexyl{[2-({[(2,6-dimethylphenyl)amino]carbonyl}amino)-4,5-dimethoxybenzoyl]amino}acetic acid. This compound was prepared as described in Example 2 except that 2,6-dimethylphenylisocyanate was substituted for 2-chloro-6-methylphenylisocyanate, and 2-amino-4,5-dimethoxybenzoic acid was substituted for 3-amino-2-naphthalenecarboxylic acid.

### Example 118

3-cyclohexyl-N-[2-({[(2,6-dimethylphenyl)amino]carbonyl}amino)-4,5-difluorobenzoyl]-L-alanine. This compound was prepared as described in Example 2 except that 2,6-dimethylphenylisocyanate was substituted for 2-chloro-6-methylphenylisocyanate, 2-amino-4,5-difluorobenzoic acid was substituted for 3-amino-2-naphthalenecarboxylic acid, and Fmoc-L-cyclohexylalanine was substituted for Fmoc-L-cyclohexylglycine.

### Example 119

3-cyclohexyl-N-[2-({[(2,6-dimethylphenyl)amino]carbonyl}amino)-4,5-dimethoxybenzoyl]-L-alanine. This compound was prepared as described in Example 2 except that 2,6-dimethylphenylisocyanate was substituted for 2-chloro-6-methylphenylisocyanate, 2-amino-4,5-dimethoxybenzoic acid was substituted for 3-amino-2-naphthalenecarboxylic acid, and Fmoc-L-cyclohexylalanine was substituted for Fmoc-L-cyclohexylglycine.

### Example 120

{[3-({[(2,6-diethylphenyl)amino]carbonyl}amino)-2-naphthoyl]amino}(phenyl)acetic acid. This compound was prepared as described in Example 2 except that 2,6-diethylphenylisocyanate was substituted for 2-chloro-6-methylphenylisocyanate, and Fmoc-L-phenylglycine was substituted for Fmoc-L-cyclohexylglycine.

### Example 121

N-[4-chloro-2-({[(2,6-diethylphenyl)amino]carbonyl}amino)benzoyl]-2-fluoro-D-phenylalanine. This compound was prepared as described in Example 2 except that 2,6-diethylphenylisocyanate was substituted for 2-chloro-6-methylphenylisocyanate, 2-amino-4-chlorobenzoic acid was substituted for 3-amino-2-naphthalenecarboxylic acid, and Fmoc-D-2-fluorophenylalanine was substituted for Fmoc-L-cyclohexylglycine.

### Example 122

N-[3-({[(2-chloro-6-methylphenyl)amino]carbonyl}amino)-2-naphthoyl]-3-cyclohexyl-L-alanine. This compound was prepared as described in Example 2 except that Fmoc-L-cyclohexylalanine was substituted for Fmoc-L-cyclohexylglycine.

### Example 123

{[3-({[(2-chloro-6-methylphenyl)amino]carbonyl}amino)-2-naphthalenyl]carbonyl}amino)(phenyl)acetic acid. This compound was prepared as described in Example 2 except that Fmoc-L-phenylglycine was substituted for Fmoc-L-cyclohexylglycine.

### Example 124

N-[3-({[(2-chloro-6-methylphenyl)amino]carbonyl}amino)-2-naphthoyl]-2-fluoro-D-phenylalanine. This compound was prepared as described in Example 2 except that Fmoc-D-2-fluorophenylalanine was substituted for Fmoc-L-cyclohexylglycine.

### Example 125

N-[4-chloro-2-({[(2-chloro-6-methylphenyl)amino]carbonyl}amino)benzoyl]-3-cyclohexyl-L-alanine. This compound was prepared as described in Example 2 except that 2-amino-4-chlorobenzoic acid was substituted for 3-amino-2-naphthalenecarboxylic acid, and Fmoc-L-cyclohexylalanine was substituted for Fmoc-L-cyclohexylglycine.

### Example 126

N-{[2-({[(2,6-dimethylphenyl)amino]carbonyl}amino)-4,5,6,7-tetrahydro-1-benzothien-3-yl]carbonyl}-L-isoleucine. This compound was prepared as described in Example 3 except that Fmoc-L-isoleucine-Wang resin was substituted for Fmoc-L-valine-Wang resin.

### Example 127

N-[(2-{[(mesitylamino)carbonyl]amino}-4,5,6,7-tetrahydro-1-benzothien-3-yl)carbonyl]-L-isoleucine. This compound was prepared as described in Example 3 except that 2,4,6-trimethylphenylisocyanate was substituted for 2,6-dimethylphenylisocyanate, and Fmoc-L-isoleucine-Wang resin was substituted for Fmoc-L-valine-Wang resin.

### Example 128

N-{[2-({[(2-chloro-6-methylphenyl)amino]carbonyl}amino)-4,5,6,7-tetrahydro-1-benzothien-3-yl]carbonyl}-L-isoleucine. This compound was prepared as described in Example 3 except that 2-chloro-6-methylphenylisocyanate was substituted for 2,6-dimethylphenylisocyanate, and Fmoc-L-isoleucine-Wang resin was substituted for Fmoc-L-valine-Wang resin.

### Example 129

N-{[2-({[(2,6-dichlorophenyl)amino]carbonyl}amino)-4,5,6,7-tetrahydro-1-benzothien-3-yl]carbonyl}-L-isoleucine. This compound was prepared as described in Example 3 except that 2,6-dichlorophenylisocyanate was substituted for 2,6-dimethylphenylisocyanate, and Fmoc-L-isoleucine-Wang resin was substituted for Fmoc-L-valine-Wang resin.

### Example 130

N-{[2-({[(2-chloro-6-methylphenyl)amino]carbonyl}amino)-4,5,6,7-tetrahydro-1-benzothien-3-yl]carbonyl}-L-leucine. This compound was prepared as described in Example 3 except that 2-chloro-6-methylphenylisocyanate was substituted for 2,6-dimethylphenylisocyanate, and Fmoc-L-leucine-Wang resin was substituted for Fmoc-L-valine-Wang resin.

### Example 131

N-{[2-({[(2,6-dichlorophenyl)amino]carbonyl}amino)-4,5,6,7-tetrahydro-1-benzothien-3-yl]carbonyl}-L-leucine. This compound was prepared as described in Example 3 except that 2,6-dichlorophenylisocyanate was substituted for 2,6-dimethylphenylisocyanate, and Fmoc-L-leucine-Wang resin was substituted for Fmoc-L-valine-Wang resin.

### Example 132

N-{[2-({[(2,6-dimethylphenyl)amino]carbonyl}amino)-4,5,6,7-tetrahydro-1-benzothien-3-yl]carbonyl}-L-aspartic acid. This compound was prepared as described in Example 3 except that Fmoc-L-Aspartic acid(tBu)-Wang resin was substituted for Fmoc-L-valine-Wang resin.

### Example 133

N-[(2-{[(mesitylamino)carbonyl]amino}-4,5,6,7-tetrahydro-1-benzothien-3-yl)carbonyl]-L-aspartic acid. This compound was prepared as described in Example 3 except that 2,4,6-trimethylphenylisocyanate was substituted for 2,6-dimethylphenylisocyanate, and Fmoc-L-Aspartic acid(tBu)-Wang resin was substituted for Fmoc-L-valine-Wang resin.

### Example 134

N-{[2-({[(2-chloro-6-methylphenyl)amino]carbonyl}amino)-4,5,6,7-tetrahydro-1-benzothien-3-yl]carbonyl}-L-aspartic acid. This compound was prepared as described in Example 3 except that 2-chloro-6-methylphenylisocyanate was substituted for 2,6-dimethylphenylisocyanate, and Fmoc-L-Aspartic acid(tBu)-Wang resin was substituted for Fmoc-L-valine-Wang resin.

### Example 135

N-{[2-({[(2-isopropyl-6-methylphenyl)amino]carbonyl}amino)-4,5,6,7-tetrahydro-1-benzothien-3-yl]carbonyl}-L-aspartic acid. This compound was prepared as described in Example 3 except that 2-isopropyl-6-methylphenylisocyanate was substituted for 2,6-dimethylphenylisocyanate, and Fmoc-L-Aspartic acid(tBu)-Wang resin was substituted for Fmoc-L-valine-Wang resin.

### Example 136

N-{[2-({[(2,6-diethylphenyl)amino]carbonyl}amino)-4,5,6,7-tetrahydro-1-benzothien-3-yl]carbonyl}-L-aspartic acid. This compound was prepared as described in Example 3 except that 2,6-diethylphenylisocyanate was substituted for 2,6-dimethylphenylisocyanate, and Fmoc-L-Aspartic acid(tBu)-Wang resin was substituted for Fmoc-L-valine-Wang resin.

### Example 137

N-{[2-({[(2,6-dichlorophenyl)amino]carbonyl}amino)-4,5,6,7-tetrahydro-1-benzothien-3-yl]carbonyl}-L-aspartic acid. This compound was prepared as described in Example 3 except that 2,6-dichlorophenylisocyanate was substituted for 2,6-dimethylphenylisocyanate, and Fmoc-L-Aspartic acid(tBu)-Wang resin was substituted for Fmoc-L-valine-Wang resin.

### Example 138

(2S)-cyclohexyl({[2-({[(2,6-dimethylphenyl)amino]carbonyl}amino)-4,5,6,7-tetrahydro-1-benzothien-3-yl]carbonyl}amino)acetic acid. This compound was prepared as described in Example 3 except that Fmoc-L-cyclohexylglycine-Wang resin (prepared as in Example 2a) was substituted for Fmoc-L-valine-Wang resin.

### Example 139

(2S)-({[2-({[(2-chloro-6-methylphenyl)amino]carbonyl}amino)-4,5,6,7-tetrahydro-1-benzothien-3-yl]carbonyl}amino)(cyclohexyl)acetic acid. This compound was prepared as described in Example 3 except that 2-chloro-6-methylphenylisocyanate was substituted for 2,6-dimethylphenylisocyanate, and Fmoc-L-cyclohexylglycine-Wang resin (prepared as in Example 2a) was substituted for Fmoc-L-valine-Wang resin.

### Example 140

(2S)-cyclohexyl({[2-({[(2,6-dichlorophenyl)amino]carbonyl}amino)-4,5,6,7-tetrahydro-1-benzothien-3-yl]carbonyl}amino)acetic acid. This compound was prepared as described in Example 3 except that 2,6-dichlorophenylisocyanate was substituted for 2,6-dimethylphenylisocyanate, and Fmoc-L-cyclohexylglycine-Wang resin (prepared as in Example 2a) was substituted for Fmoc-L-valine-Wang resin.

### Example 141

(2S)-cyclohexyl({[2-({[(2-methylphenyl)amino]carbonyl}amino)-1-benzothien-3-yl]carbonyl}amino)acetic acid. This compound was prepared as described in Example 4 except that 2-methylphenylisocyanate was substituted for 2,6-dimethylphenylisocyanate.

### Example 142

(2S)-({[2-({[(2-chloro-6-methylphenyl)amino]carbonyl}amino)-1-benzothien-3-yl]carbonyl}amino)(cyclohexyl)acetic acid. This compound was prepared as described in Example 4 except that 2-chloro-6-methylphenylisocyanate was substituted for 2,6-dimethylphenylisocyanate.

### Example 143

(2S)-cyclohexyl{[3-({[(2-methylphenyl)amino]carbonyl}amino)-2-naphthoyl]amino}acetic acid. This compound was prepared as described in Example 5 except that 2-methylphenylisocyanate was substituted for 2-chloro-6-methylphenylisocyanate, and Fmoc-L-cyclohexylglycine-Wang resin was substituted for Fmoc-(1-Boc-piperidin-3-yl)-D,L-glycine-Wang resin.

### Example 144

3-cyclohexyl-N-{[3-({[(2-methylphenyl)amino]carbonyl}amino)-2-naphthalenyl]carbonyl}-L-alanine. This compound was prepared as described in Example 5 except that 2-methylphenylisocyanate was substituted for 2-chloro-6-methylphenylisocyanate, and Fmoc-L-cyclohexylalanine-Wang resin was substituted for Fmoc-(1-Boc-piperidin-3-yl)-D,L-glycine-Wang resin.

### Example 145

3-cyclohexyl-N-{[3-({[(2,6-dimethylphenyl)amino]carbonyl}amino)-2-naphthalenyl]carbonyl}-L-alanine. This compound was prepared as described in Example 5 except that 2,6-dimethylphenylisocyanate was substituted for 2-chloro-6-methylphenylisocyanate, and Fmoc-L-cyclohexylalanine-Wang resin was substituted for Fmoc-(1-Boc-piperidin-3-yl)-D,L-glycine-Wang resin.

### Example 146

3-cyclohexyl-N-{[3-({[(2,6-dichlorophenyl)amino]carbonyl}amino)-2-naphthalenyl]carbonyl}-L-alanine. This compound was prepared as described in Example 5 except that 2,6-dichlorophenylisocyanate was substituted for 2-chloro-6-methylphenylisocyanate, and Fmoc-L-cyclohexylalanine-Wang resin was substituted for Fmoc-(1-Boc-piperidin-3-yl)-D,L-glycine-Wang resin.

### Example 147

N-{[3-({[(3,5-dimethyl-4-isoxazolyl)amino]carbonyl}amino)-2-naphthalenyl]carbonyl}-L-aspartic acid. This compound was prepared as described in Example 5 except that 3,5-dimethylisoxazole-4-isocyanate was substituted for 2-chloro-6-methylphenylisocyanate, and Fmoc-L-Aspartic acid(tBu)-Wang resin was substituted for Fmoc-(1-Boc-piperidin-3-yl)-D,L-glycine-Wang resin.

### Example 148

N-{[3-({[(2,6-dichlorophenyl)amino]carbonyl}amino)-2-naphthalenyl]carbonyl}-L-aspartic acid. This compound was prepared as described in Example 5 except that 2,6-dichlorophenylisocyanate was substituted for 2-chloro-6-methylphenylisocyanate, and Fmoc-L-Aspartic acid(tBu)-Wang resin was substituted for Fmoc-(1-Boc-piperidin-3-yl)-D,L-glycine-Wang resin.

### Example 149

N-{[3-({[(2,6-difluorophenyl)amino]carbonyl}amino)-2-naphthalenyl]carbonyl}-L-aspartic acid. This compound was prepared as described in Example 5 except that 2,6-difluorophenylisocyanate was substituted for 2-chloro-6-methylphenylisocyanate, and Fmoc-L-Aspartic acid(tBu)-Wang resin was substituted for Fmoc-(1-Boc-piperidin-3-yl)-D,L-glycine-Wang resin.

### Example 150

N-{[3-({[(2,6-dimethylphenyl)amino]carbonyl}amino)-2-naphthalenyl]carbonyl}-L-aspartic acid. This compound was prepared as described in Example 5 except that 2,6-dimethylphenylisocyanate was substituted for 2-chloro-6-methylphenylisocyanate, and Fmoc-L-Aspartic acid(tBu)-Wang resin was substituted for Fmoc-(1-Boc-piperidin-3-yl)-D,L-glycine-Wang resin.

### Example 151

N-{[3-({[(2-chlorophenyl)amino]carbonyl}amino)-2-naphthalenyl]carbonyl}-L-aspartic acid. This compound was prepared as described in Example 5 except that 2,6-chlorophenylisocyanate was substituted for 2-chloro-6-methylphenylisocyanate, and Fmoc-L-Aspartic acid(tBu)-Wang resin was substituted for Fmoc-(1-Boc-piperidin-3-yl)-D,L-glycine-Wang resin.

### Example 152

N-{[3-({[(2-methylphenyl)amino]carbonyl}amino)-2-naphthalenyl]carbonyl}-L-aspartic acid. This compound was prepared as described in Example 5 except that 2-methylphenylisocyanate was substituted for 2-chloro-6-methylphenylisocyanate, and Fmoc-L-Aspartic acid(tBu)-Wang resin was substituted for Fmoc-(1-Boc-piperidin-3-yl)-D,L-glycine-Wang resin.

### Example 153

(2S)-cyclohexyl({[3-({[(2,6-difluorophenyl)amino]carbonyl}amino)-2-naphthalenyl]carbonyl}amino)ethanoic acid. This compound was prepared as described in Example 5 except that 2,6-difluorophenylisocyanate was substituted for 2-chloro-6-methylphenylisocyanate, and Fmoc-L-cyclohexylalanine-Wang resin was substituted for Fmoc-(1-Boc-piperidin-3-yl)-D,L-glycine-Wang resin.

### Example 154

(2S)-cyclohexyl({[3-({[(2,6-dichlorophenyl)amino]carbonyl}amino)-2-naphthalenyl]carbonyl}amino)acetic acid. This compound was prepared as described in Example 5 except that 2,6-dichlorophenylisocyanate was substituted for 2-chloro-6-methylphenylisocyanate, and Fmoc-L-cyclohexylalanine-Wang resin was substituted for Fmoc-(1-Boc-piperidin-3-yl)-D,L-glycine-Wang resin.

### Example 155

(2S)-cyclohexyl{[(3-{[(2,6-dichlorophenyl)acetyl]amino}-2-naphthalenyl)carbonyl]amino}ethanoic acid. This compound was prepared as described in Example 7 except that (2,6-dichlorophenyl)acetyl chloride was substituted for (2-methylphenyl)acetyl chloride.

### Example 156

(2S)({[4chloro2({[(2,6dichlorophenyl)amino]carbonyl}amino) phenyl]carbonyl}amino)(cyclohexyl)ethanoic acid.

### Step 1

### 4-chloro-2-({[(2,6-dichlorophenyl)amino]carbonyl}amino)benzoic acid

2,6-Dichlorophenyl isocyanate (0.60 g, 3.21 mmol) was added to a solution of 4-chloroanthranilic acid (0.50 g, 2.91 mmol) and triethylamine (0.59 g, 5.82 mmol) in 20 mL of DMF. The mixture was heated at 70° C. for 2 hours. The cooled reaction mixture was acidified with 10 mL of 1N HCl, and filtered to collect the precipitated white solid. After washing with water and drying under vacuum 0.616 g (59% yield) of desired product was obtained. ES-MS m/z 358

### Step 2

### Methyl(2S)({[4chloro2({[(2,6dichlorophenyl)amino]carbonyl}amino)phenyl]carbonyl}amino)(cyclohexyl)ethanoate

HATU (0.319 g, 0.84 mmol) was added to a solution of 4-chloro-2-({[(2,6-dichlorophenyl)amino]carbonyl}amino)benzoic acid (0.200 g, 0.56 mmol), methyl(2S) -amino(cyclohexyl)ethanoate hydrochloride (0.115 g, 0.56 mmol) and diisopropylethylamine (0.11 g, 0.84 mmol) in 10 mL of DMF. After stirring at RT (room temperature) overnight, the mixture was diluted with ethyl acetate and water. The organic layer was washed with water and brine, dried over sodium sulfate, filtered, the solvent evaporated. Chromatography on silica gel with hexane/ethyl acetate gave 0.195 g of 80% pure product.

### Step 3

### (2S)({[4chloro2({[(2,6dichlorophenyl)amino]carbonyl}amino) phenyl]carbonyl}amino)(cyclohexyl)ethanoic acid

Lithium hydroxide (0.089 g, 3.70 mmol) was added to a solution of methyl(2S)({[4chloro2({[(2,6dichlorophenyl)amino]carbonyl}amino)phenyl]carbonyl}amino)(cyclohexyl)ethanoate (0.190 g, 0.37 mmol) in THF:MeOH:water/4:1:1. The mixture was stirred at RT overnight. The reaction mixture was acidified with 1N aqueous HCl and evaporated to dryness. The residue was extracted between dichloromethane and water. The organic phase was dried over sodium sulfate and concentrated to dryness. The residue was purified by chromatography on silica gel with dichloromethane/methanol to give 12 mg (6.5% yield) of pure desired product as a white solid. ES MS m/z 496 (M−H).

### Example 157

### (2S)-({[4-chloro-2-({[(2,6-dimethylphenyl)amino]carbonyl}amino) phenyl]carbonyl}amino)(cyclohexyl)ethanoic acid

This compound was synthesized by methods similar to those described as in Example 156 in 1% overall yield using 2,6-dimethylphenyl isocyanate in place of 2,6-dichlorophenyl isocyanate. ES MS m/z 456 (M−H)

### Example 158

### (2S)-cyclohexyl{[3({[(2,4,6trichlorophenyl)amino]carbonyl}amino)-2-naphthoyl]amino}ethanoic acid

### Step 1

### 3-[(tert-butoxycarbonyl)amino]-2-naphthoic acid

Di-tert-butyl-dicarbonate (1.75 g, 8.01 mmol) was added to 3-amino-2-naphthoic acid (1.0 g, 5.34 mmol) in 20 mL of THF and 20 mL of 1N aqueous sodium hydroxide. The mixture was stirred at RT for ca. 20 h. The THF was removed under reduced pressure and the aqueous phase as acidified with 1N aqueous NaHSO₄. The resulting solution was extracted with ethyl acetate. The organic phase was dried over sodium sulfate and concentrated to dryness. The residue was purified by chromatography on silica gel with dichloromethane/methanol to give 1.1 g (71% yield) of desired product as a off white solid. ES MS m/z 286 (M−H).

### Step 2

### Methyl(2S)-({3-[(tert-butoxycarbonyl)amino]-2-naphthoyl}amino)(cyclohexyl)ethanoate

HATU (0.595 g, 1.56 mmol) was added to a solution of 3-[(tert-butoxycarbonyl)amino]-2-naphthoic acid (0.390 g, 1.36 mmol), methyl(2S)-amino(cyclohexyl)ethanoate hydrochloride (0.325 g, 1.56 mmol) and diisopropylethylamine (0.263 g, 2.04 mmol) in 10 mL of DMF. The mixture was stirred at RT for ca. 3 h. The DMF was removed under reduced pressure and the residue was diluted with ethyl acetate and water. The organic layer was washed with water and brine, dried over sodium sulfate, filtered, and the solvent evaporated. Chromatography on silica gel with hexane/ethyl acetate gave 0.443 g of product.

### Step 3

### Methyl(2S)-[(3-amino-2-naphthoyl)amino](cyclohexyl)ethanoate hydrochloride

Methyl(2S)-({3-[(tert-butoxycarbonyl)amino]-2-naphthoyl}amino)(cyclohexyl)ethanoate (0.44 g, 1.0 mmol) in 10 mL of CH₂Cl₂ was treated with 5 mL of 4 N HCl in dioxane. The mixture as stirred at RT for ca. 1.5 h and the solvents were removed under reduced pressure to give the 0.376 g (100%) of the product.

### Step 4

### Methyl(2S)-cyclohexyl{[3-({[(2,4,6-trichlorophenyl)amino]carbonyl}amino)-2-naphthoyl]amino}ethanoate

Methyl(2S)-[(3-amino-2-naphthoyl) amino](cyclohexyl)ethanoate hydrochloride (0.05 g, 0.133 mmol) in 5 mL of pyridine was treated with 2,4,6-trichlorophenyl isocyanate (0.15 g, 0.67 mmol) for ca. 4 h at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.052 g of product.

### Step 5

### (2S)-cyclohexyl{[3-({[(2,4,6-trichlorophenyl)amino]carbonyl}amino)-2-naphthoyl]amino}ethanoic acid

Lithium hydroxide monohydrate (0.0.018 g, 3.70 mmol) was added to a solution of methyl(2S)-cyclohexyl{[3-({[(2,4,6-trichlorophenyl)amino]carbonyl}amino)-2-naphthoyl]amino}ethanoate (0.052 g, 0.09 mmol) in THF:MeOH:water/3:1:1. The mixture was stirred at RT overnight. The reaction mixture was acidified with 1N aqueous HCl and extracted with ethyl acetate. The organic phase was dried over sodium sulfate and concentrated to dryness to give 42 mg (82% yield) of desired product as a white solid. ES MS m/z 546 (M−H).

### Example 159

### (2S)-cyclohexyl{[3-({[(2-ethyl-6-methylphenyl)amino]carbonyl}amino)-2-naphthoyl]amino}ethanoic acid

This compound was synthesized by methods similar to those described as in Example 158 in 65% overall yield using 2-ethyl-6-methylphenyl isocyanate in place of 2,4,6-trichlorophenylisocyanate. ES MS m/z 486 (M−H).

### Example 160

### (2S)-({3-[({[2-chloro-6-(trifluoromethyl)phenyl]amino}carbonyl) amino]-2-naphthoyl}amino)(cyclohexyl)ethanoic acid

This compound was synthesized by methods similar to those described as in Example 158 in 70% overall yield using 2-chloro-6-trifluoromethylphenyl isocyanate in place of 2,4,6-trichlorophenylisocyanate. ES MS m/z 546 (M−H).

### Example 161

### (2S)-cyclohexyl{[3-({[2,6-dichloro-4-(trifluoromethyl) phenyl]acetyl}amino)-2-naphthoyl]amino}ethanoic acid

### Step 1

### Methyl(2S)-cyclohexyl{[3-({[2,6-dichloro-4-(trifluoromethyl)phenyl]acetyl}amino)-2-naphthoyl]amino}ethanoate

HATU (0.058 g, 0.15 mmol) was added to a solution of methyl(2S)-[(3-amino-2-naphthoyl)amino](cyclohexyl)ethanoate hydrochloride (prepared as described in Example 158) (0.0.05 g, 0.133 mmol), [2,6-dichloro-4-(trifluoromethyl) phenyl]acetic acid (0.042 g, 0.15 mmol) and diisopropylethylamine (0.03 g, 0.20 mmol) in 3 mL of DMF. The mixture was stirred at RT for ca. 20 h. The DMF was removed under reduced pressure and the residue was diluted with ethyl acetate. The organic layer was washed with NaHCO₃ and brine, dried over sodium sulfate, filtered, and the solvent evaporated. Chromatography on silica gel with hexane/ethyl acetate gave 0.042 g of product.

### Step 2

### (2S)-cyclohexyl{[3-({[2,6-dichloro-4-(trifluoromethyl)phenyl]acetyl}amino)-2-naphthoyl]amino}ethanoic acid

Lithium hydroxide monohydrate (0.0.009 g, 0.2 mmol) was added to a solution methyl(2S)-cyclohexyl{[3-({[2,6-dichloro-4-(trifluoromethyl)phenyl]acetyl}amino)-2-naphthoyl]amino}ethanoate (0.040 g, 0.07 mmol) in THF:MeOH:water/3:1:1. The mixture was stirred at RT overnight. The reaction mixture was acidified with 1N aqueous HCl and extracted with ethyl acetate. The organic phase was dried over sodium sulfate and concentrated to dryness to give 38 mg (97% yield) of desired product as a white solid. ES MS m/z 579 (M−H).

### Example 162

### (2S)-cyclohexyl[(3-{[(2,4,6-trichlorophenyl)acetyl]amino}-2-naphthoyl)amino]ethanoic acid

This compound was synthesized by similar methods to those described as in Example 161 in 45% overall yield using (2,4,6-trichlorophenyl)acetic acid in place of [2,6-dichloro-4-(trifluoromethyl)phenyl]acetic acid. ES MS m/z 546 (M−H).

### Example 163

### N-[3-({[(2,6-dimethylphenyl)amino]carbonyl}amino)-2-naphthoyl]-beta-alanine

### Step 1

### 3-({[(2,6-dimethylphenyl)amino]carbonyl}amino)-2-naphthoic acid

To a DMF solution (50 mL) containing 3-amino-2 napthoic acid (2 g, 10.68 mmol) was added TEA (3 mL, 21.36 mmol). After stirring for 30 min, 2,6-dimethylphenyl isocyanate (1.72 mL, 11.75 mmol) was added and the solution heated at 75° C. for 2 h. After cooling to RT the mixture was acidified with 1.0M HCl and extracted with ethyl acetate. A white precipitate was observed in the organic layer and was separated by filtration. The resulting solid was identified as the product by proton NMR and was taken on without further purification. The product was isolated as a white solid in a 94% yield.

### Step 2

### Methyl N-[3-({[(2,6-dimethylphenyl)amino]carbonyl}amino)-2-naphthoyl]-beta-alaninate

To a DMF solution (5 mL) of 3-({[(2,6-dimethylphenyl)amino]carbonyl}amino)-2-naphthoic acid (0.2 g, 0.597 mmol) was added HATU (0.27 g, 0.717 mmol) and DIEA (0.124 mL, 0.717 mmol). After stirring for 30 min, beta-alanine methylester hydrochloride (0.1 g, 0.717 mmol) in DMF (2 mL) was added. After 2 h at RT the reaction was poured in sat. NaHCO₃ and extracted with ethyl acetate. The combined organics were then washed with water, dried over MgSO₄, filtered and reduced in vacuo to yield a yellow solid. The solid was purified using flash chromotography (EtOAc/Hexanes). The product was isolated as a white solid in a 62% yield.

### Step 3

### N-[3-({[(2,6-dimethylphenyl)amino]carbonyl}amino)-2-naphthoyl]-beta-alanine

To a THF soln (5 mL) containing methyl N-[3-({[(2,6-dimethylphenyl)amino]carbonyl}amino)-2-naphthoyl]-beta-alaninate (0.15 g, 0.357 mmol) was added LiOH (0.085 g, 3.57 mmol) in a 2 mL soln of H₂O+1 mL of MeOH. The soln was allowed to stir at RT for 2 h. To the mixture was added 1.0 M HCl and extracted with ethyl acetate. The combined organics were dried over MgSO₄, filtered and reduced in vacuo to yield a white solid. The solution was triturated with ether and filtered to yield the product as a white solid in a 35% yield. ES MS m/z 404 (M−H).

### Example 164

### (2S)-cyclohexyl[(3-{[(mesitylamino)carbonyl]amino}-2-naphthoyl)amino]ethanoic acid

### Step 1

### 3-{[(mesitylamino)carbonyl]amino}-2-naphthoic acid

The title compound was prepared in 65% yield as described in Example 163, Step 1, except that 2,4,6-trimethylphenylisoycanate was substituted for 2,6-dimethylphenyl isocyanate.

### Step 2

### Methyl(2S)-cyclohexyl[(3-{[(mesitylamino)carbonyl]amino}-2-naphthoyl)amino]ethanoate

The title compound was prepared in 65% yield as described in Example 163, Step 2, except that 3-{[(mesitylamino)carbonyl]amino}-2-naphthoic acid was substituted for 3-({[(2,6-dimethylphenyl)amino]carbonyl}amino)-2-naphthoic acid and methyl(2S)-amino(cyclohexyl)ethanoate hydrochloride was substituted for beta-alanine methyl ester hydrochloride.

### Step 3

### (2S)-cyclohexyl[(3-{[(mesitylamino)carbonyl]amino}-2-naphthoyl)amino]ethanoic acid

The title compound was prepared in 40% yield as described in Example 163, Step 3, except that methyl(2S)-cyclohexyl[(3-{[(mesitylamino)carbonyl]amino}-2-naphthoyl)amino]ethanoate was substituted for methyl N-[3-({[(2,6-dimethylphenyl)amino]carbonyl}amino)-2-naphthoyl]-beta-alaninate and 1,4-dioxane was substituted for THF. ESMS m/z 486 (M−H).

### Example 165

### 4-Chloro-2-({[(2,6-dichlorophenyl)amino]carbonyl}amino)benzoic acid

Triethylamine (0.81 mL, 5.82 mmol) was added to a solution of 2-amino-4-chlorobenzoic acid (0.50 g, 2.91 mmol) in 20 mL of DMF. After stirring at room temperature for 15 minutes, 2,6-dichlorophenylisocyanate (0.60 g, 3.21 mmol) was added. The mixture was heated at 75° C. for 2 hours. After cooling to room temperature, 1N HCl (10 mL) was added, and the mixture was extracted with ethyl acetate. The organic layer was concentrated under vacuum to give 0.616 g (59% yield) of desired product as a white powder. ES MS m/z 358 (M−H).

### Example 166

### 2-({[(2,6-Dimethylphenyl)amino]carbonyl}amino)benzoic acid

Triethylamine (1.6 mL, 11.7 mmol) was added to a solution of 2-amino-4-chlorobenzoic acid (1.00 g, 5.83 mmol) in 30 mL of DMF. After stirring at room temperature for 30 minutes, 2,6-dimethylphenylisocyanate (0.94 g, 6.41 mmol) was added. The mixture was heated at 75° C. for 1 hour. After cooling to room temperature, 1N HCl (15 mL) was added. The precipitated solid was poorly soluble in ethyl acetate. The solid was collected by filtration, washed with water and dried under vacuum to give 1.58 g (85% yield) of desired product. ES MS m/z 317 (M−H).

### Example 167

### (2S)-({[4-Chloro-2-({[(2,6-dimethylphenyl)amino]carbonyl}amino)phenyl]carbonyl}amino)(cyclohexyl)-ethanoic acid

### Step 1

### Methyl(2S)-({[4-chloro-2-({[(2,6-dimethylphenyl)amino]carbonyl}amino)phenyl]carbonyl}amino)(cyclohexyl)ethanoate

HATU (0.179 g, 0.47 mmol) was added to a solution of 2-({[(2,6-dimethylphenyl)amino]carbonyl}amino)benzoic acid (0.100 g, 0.31 mmol), in 5 mL of DMF. After stirring for 30 minutes, methyl(2S)-amino(cyclohexyl)ethanoate hydrochloride (0.064 g, 0.31 mmol) and diisopropylethylamine (0.081 mL, 0.46 mmol) was added. The mixture was stirred at room temperature overnight. The DMF was removed under vacuum and the residue was extracted between ethyl acetate and water. The organic phase was dried over anhydrous sodium sulfate and the solvent was removed under vacuum. Chromatography on silica gel with hexane/ethyl acetate gave 0.079 g (54% yield) of desired product as a colorless gum.

### Step 2

### (2S)-({[4-Chloro-2-({[(2,6-dimethylphenyl)amino]carbonyl}amino)phenyl]carbonyl}amino)(cyclohexyl)-ethanoic acid

A solution of lithium hydroxide (0.040 g, 1.70 mmol) in 0.5 mL of water was added to a solution of methyl(2S)-({[4-chloro-2-({[(2,6-dimethylphenyl)amino]carbonyl}amino)phenyl]carbonyl}amino)(cyclohexyl)ethanoate (0.079 g, 0.17 mmol) in THF:methanol/4:1. The mixture was heated at 50° C. overnight. The reaction mixture was acidified with 1N aqueous HCl and the solvent was evaporated to dryness. The residue was extracted between water and dichloromethane. The organic phase was dried over sodium sulfate and the solvent was removed under vacuum to give 0.025 g (32% yield) of desired product as a white solid. ES MS m/z 456 (M−H).

### Example 168

### (2S)-({[4-Chloro-2-({[(2,6-dichlorophenyl)amino]carbonyl}amino)phenyl]carbonyl}amino)(cyclohexyl)-ethanoic acid

### Step 1

### Methyl(2S)-({[4-chloro-2-({[(2,6-dichlorophenyl)amino]carbonyl}amino)phenyl]carbonyl}amino)(cyclohexyl)ethanoate

HATU (0.319 g, 0.84 mmol) was added to a solution of 2-({[(2,6-dichlorophenyl)amino]carbonyl}amino)benzoic acid (0.200 g, 0.56 mmol), methyl(2S)-amino(cyclohexyl)ethanoate hydrochloride (0.115 g, 0.56 mmol) and diisopropylethylamine (0.15 mL, 0.84 mmol) in 10 mL of DMF. The mixture was stirred at room temperature overnight. The reaction mixture was extracted between ethyl acetate and water. The organic phase was washed with water and brine and dried over anhydrous sodium sulfate and the solvent was removed under vacuum. Chromatography on silica gel with hexane/ethyl acetate gave a mixture containing 80% of desired product. This material was carried on to the next step without additional purification.

### Step 2

### (2S)-({[4-Chloro-2-({[(2,6-dichlorophenyl)amino]carbonyl}amino)phenyl]carbonyl}amino)(cyclohexyl)-ethanoic acid

A solution of lithium hydroxide (0.089 g, 3.70 mmol) in 1 mL of water was added to a solution of methyl(2S)-({[4-chloro-2-({[(2,6-dichlorophenyl)amino]carbonyl}amino)phenyl]carbonyl}amino)(cyclohexyl)ethanoate (0.190 g, 0.37 mmol) in 5 ml of THF:methanol/4:1. The mixture was heated at 50° C. for 2 hours. The solvent was evaporated and the residue was treated with aqueous 1N hydrochloric acid and extracted with dichloromethane. The organic phase was dried over sodium sulfate and the solvent was evaporated. Chromatography on silica gel with dichloromethane/methanol gave 0.012 g (6.5% yield) ES MS m/z 496 (M−H).

### Example 169

### (2S)Cyclohexyl({[2({[(2,6dimethylphenyl)amino]carbonyl}amino)-5-methylphenyl]carbonyl}amino)ethanoic acid

### Step 1

### 2-({[(2,6-dimethylphenyl)amino]carbonyl}amino)-5-methylbenzoic acid

Triethylamine (0.81 mL, 5.82 mmol) and 2,6-dimethylphenylisocyanate (0.47 g, 3.21 mmol) were added to a solution of 2-amino-5-methylbenzoic acid (0.500 g, 3.3 mmol) in anhydrous DMF (15 mL). The mixture was heated at 70° C. for one hour. After cooling to room temperature, 2 mL of 6N aqueous HCl was added and the mixture was diluted with water. The precipitated solid was filtered, washed with water and dried under vacuum overnight to give 0.96 g of desired product as a white powder.

### Step 2

### Methyl(2S)-cyclohexyl({[2({[(2,6dimethylphenyl)amino]carbonyl}amino)-5-methylphenyl]carbonyl}amino)ethanoate

HATU (0.191 g, 0.50 mmol) was added to a solution of 2-({[(2,6-dimethylphenyl)amino]carbonyl}amino)-5-methylbenzoic acid (0.100 g, 0.33 mmol), methyl(2S)-amino(cyclohexyl)ethanoate hydrochloride (0.070 g, 0.33 mmol) and diisopropylethylamine (0.087 mL, 0.50 mmol) in 5 mL of DMF. After stirring at room temperature overnight, the reaction mixture was diluted with ethyl acetate and washed with water and brine. The organic phase was dried over sodium sulfate and the solvent was evaporated. Chromatography on silica gel with hexane/ethyl acetate gave 0.066 g (44% yield) of desired product as a white solid.

### Step 3

### (2S)Cyclohexyl({[2({[(2,6dimethylphenyl)amino]carbonyl}amino)-5-methylphenyl]carbonyl}amino)ethanoic acid

Lithium hydroxide (0.034 g, 1.41 mmol) was added to a solution of methyl(2S)-cyclohexyl({[2({[(2,6dimethylphenyl)amino]carbonyl}amino)-5-methylphenyl]carbonyl}amino)ethanoate (0.064 g, 0.14 mmol) in 3 mL of THF:methanol:water/4:1:1. The mixture was stirred at room temperature for 4 hours and acidified with 1N aqueous HCl. The solvents were evaporated and the residue was extracted between dichloromethane and water. An insoluble white solid remained in suspension which was filtered and dried under vacuum to give 0.039 g (64% yield) of desired product. ES MS m/z 436 (M−H).

### Example 170

### N{[4chloro2({[(2,6dimethylphenyl)amino]carbonyl}amino)phenyl]carbonyl}glycine

### Step 1

### 1,1-Dimethylethyl N-{[4-chloro-2-({[(2,6-dimethylphenyl)amino]carbonyl}amino)phenyl]carbonyl}glycinate

HATU (0.177 g, 0.46 mmol) was added to a solution of 2-({[(2,6-dimethylphenyl)amino]carbonyl}amino)-4-chlorobenzoic acid (0.100 g, 0.31 mmol), 1,1-dimethylethyl glycinate (0.061 g, 0.46 mmol) and diisopropylethylamine (0.11 mL, 0.62 mmol) in 5 mL of DMF. After stirring at room temperature for 2 hours, the reaction mixture was diluted with ethyl acetate and washed with water and brine. The organic phase was dried over sodium sulfate and the solvent was evaporated. Chromatography on silica gel with hexane/ethyl acetate gave 0.076 g (57% yield) of desired product as a white solid.

### Step 2

### N-{[4-chloro-2-({[(2,6-dimethylphenyl)amino]carbonyl}amino)phenyl]carbonyl}glycine

Trifluoroacetic acid (0.040 mL, 0.53 mmol) was added to a solution of 1,1-Dimethylethyl N-{[4-chloro-2-({[(2,6-dimethylphenyl)amino]carbonyl}amino)phenyl]carbonyl}glycinate (0.076 g, 0.18 mmol) in 1 mL of dichloromethane. The solution was stirred at room temperature for 60 hours. The crude product was purified by chromatography on silica gel with hexane/ethyl acetate to give 0.037 g (55% yield) of the desired product as a white solid. ES MS m/z 374 (M−H).

### Example 171

### (2S)-({[4-Chloro-2-({[(2,4,6-trichlorophenyl)amino]carbonyl}amino)phenyl]carbonyl}amino)(cyclohexyl)-ethanoic acid

### Step 1

### Methyl(2S)-{[(2-amino-4-chlorophenyl)carbonyl]amino}-(cyclohexyl)ethanoate

HATU (1.66 g, 4.36 mmol) was added to a solution of 2-amino-4-chlorobenzoic acid (0.50 g, 2.91 mmol), methyl(2S)-amino(cyclohexyl)ethanoate hydrochloride (2.54 g, 12.2 mmol) and diisopropylethylamine (0.76 mL, 4.36 mmol) in 25 mL of DMF. The mixture was stirred at room temperature overnight, diluted with ethyl acetate and washed with water and brine. The organic phase was dried over anhydrous sodium sulfate and the solvent was removed under vacuum. Chromatography on silica gel with hexane/ethyl acetate gave 0.66 g (70% yield) of a white solid.

### Step 2

### Methyl(2S)-({[4-chloro-2-({[(2,4,6-trichlorophenyl)amino]carbonyl}amino)phenyl]carbonyl}amino)(cyclohexyl)ethanoate

2,4,6-Trichlorophenylisocyanate (0.343 g, 1.54 mmol) was added to a solution of methyl(2S)-{[(2-amino-4-chlorophenyl)carbonyl]amino}(cyclohexyl)ethanoate (0.100 g, 0.31 mmol) in anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent was removed under reduced pressure. Chromatography on silica gel with hexane/ethyl acetate gave 0.160 g of desired product.

### Step 3

### 2S)-({[4-Chloro-2-({[(2,4,6-trichlorophenyl)amino]carbonyl}amino)phenyl]carbonyl}amino)(cyclohexyl)-ethanoic acid

Lithium hydroxide (0.068 g, 2.8 mmol) was added to a solution of methyl(2S)-({[4-chloro-2-({[(2,4,6-trichlorophenyl)amino]carbonyl}amino)phenyl]carbonyl}amino)(cyclohexyl)ethanoate (0.155 g, 0.28 mmol) in THF:methanol:water/4:1:1. The mixture was stirred at room temperature overnight, acidified with 1N aqueous HCl, and the solvent was removed under vacuum. The residue was extracted between ethyl acetate and water. The organic phase was dried over anhydrous sodium sulfate and the solvent was removed under vacuum. Chromatography on silica gel with hexane/ethyl acetate gave 0.026 g (17% yield) of desired product as a white solid. ES MS m/z 532 (M−H).

### Example 172

### (2S)-({[4-Chloro-2-({[(2-chloro-6-methylphenyl)amino]carbonyl}-amino)phenyl]carbonyl}amino)(cyclohexyl)ethanoic acid

### Step 1

### Methyl(2S)-({[4-chloro-2-({[(2-chloro-6-methylphenyl)amino]carbonyl}-amino)phenyl]carbonyl}amino)(cyclohexyl)ethanoate

2-Chloro-6-methylphenylisocyanate (0.26 g, 1.54 mmol) was added to a solution of methyl(2S)-{[(2-amino-4-chlorophenyl)carbonyl]amino}(cyclohexyl)ethanoate (0.100 g, 0.31 mmol) in anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent was evaporated under reduced pressure. Chromatography on silica gel with hexane/ethyl acetate gave 0.158 g of desired product as a clear resin.

### Step 2

### (2S)-({[4-chloro-2-({[(2-chloro-6-methylphenyl)amino]carbonyl}-amino)phenyl]carbonyl}amino)(cyclohexyl)ethanoic acid

Lithium hydroxide (0.073 g, 3.0 mmol) was added to a solution of methyl(2S)-({[4-chloro-2-({[(2-chloro-6-methylphenyl)amino]carbonyl}amino)phenyl]-carbonyl}amino)(cyclohexyl)ethanoate (0.150 g, 0.30 mmol) in THF:methanol:water/4:1:1. The mixture was stirred at room temperature overnight, acidified with 1N aqueous HCl, and the solvent was removed under vacuum. The residue was extracted between ethyl acetate and water. The organic phase was dried over anhydrous sodium sulfate and the solvent was removed under vacuum. Chromatography on silica gel with hexane/ethyl acetate gave 0.038 g (26% yield) of desired product as a white solid. ES MS m/z 476 (M−H).

### Example 173

### (2S)-({[4-Bromo-2-({[(2,6-dimethylphenyl)amino]carbonyl}amino)phenyl]carbonyl}amino)(cyclohexyl)-ethanoic acid

### Step 1

### 4-Bromo-2-nitrobenzoic acid

Sodium carbonate (4.53 g, 42 mmol) was added to a suspension of 4-bromo-2-nitrotoluene (2.00 g, 9.26 mmol) in 140 mL of water. The mixture was heated to 80° C. Potassium permanganate (5.85 g, 37 mmol) was added and the temperature was raised to 105° C. and heating was continued under a reflux condenser overnight. The reaction mixture was cooled to room temperature and filtered through Celite. The filtrate was acidified with 6N aqueous HCl and extracted with ethyl acetate. The organic layer was dried over anhydrous sodium sulfate and the solvent was evaporated to give 0.59 g (26% yield) of desired product as a beige solid.

### Step 2

### Methyl(2S)-{[(4-bromo-2-nitrophenyl)carbonyl]amino}(cyclohexyl)ethanoate

HATU (1.35 g, 3.55 mmol) was added to a solution of 4-bromo-2-nitrobenzoic acid (0.585 g, 2.37 mmol), methyl(2S)-amino(cyclohexyl)ethanoate hydrochloride (0.592 g, 2.85 mmol) and diisopropylethylamine (0.62 mL, 3.55 mmol) in 25 mL of DMF. The mixture was stirred at room temperature overnight, diluted with ethyl acetate and washed with water and brine. The organic phase was dried over anhydrous sodium sulfate and the solvent was removed under vacuum. Chromatography on silica gel with hexane/ethyl acetate gave 0.704 g (74% yield) of desired product as a white solid.

### Step 3

### Methyl(2S)-{[(2-amino-4-bromophenyl)carbonyl]amino}(cyclohexyl)ethanoate

Tin(IV) chloride dihydrate (3.37 g, 14.9 mmol) was added to a suspension of methyl(2S)-{[(4-bromo-2-nitrophenyl)carbonyl]amino}(cyclohexyl)ethanoate (0.595 g, 1.49 mmol) in 20 mL of methanol. The mixture was heated at reflux for 5 hours. The solvent was evaporated, the residue was shaken with ethyl acetate and water and filtered through Celite. The organic layer was washed with water and brine and dried over sodium sulfate. The solvent was removed under vacuum to give 0.370 g (67% yield) of desired product.

### Step 4

### Methyl(2S)-({[4-bromo-2-({[(2,6-dimethylphenyl)amino]carbonyl}amino)phenyl]carbonyl}amino)(cyclohexyl)ethanoate

2,6-Dimethylphenylisocyanate (0.49 g, 4.92 mmol) was added to a solution of methyl(2S)-{[(2-amino-4-bromophenyl)carbonyl]amino}(cyclohexyl)ethanoate (0.363 g, 0.98 mmol) in 15 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent was removed under reduced pressure. Chromatography on silica gel with hexane/ethyl acetate gave 0.409 g (81% yield) of desired product as a white solid.

### Step 5

### (2S)-({[4-Bromo-2-({[(2,6-dimethylphenyl)amino]carbonyl}amino)phenyl]carbonyl}amino)(cyclohexyl)ethanoic acid

Lithium hydroxide (0.046 g, 1.90 mmol) was added to a solution of methyl(2S)-({[4-bromo-2-({[(2,6-dimethylphenyl)amino]carbonyl}amino)phenyl]-carbonyl}amino)(cyclohexyl)ethanoate (0.100 g, 0.19 mmol) in THF:methanol:water/4:1:1. The mixture was stirred at room temperature overnight, acidified with 1N aqueous HCl, and the solvent was removed under vacuum. The residue was extracted between ethyl acetate and water. The organic phase was dried over anhydrous sodium sulfate and the solvent was removed under vacuum to give 0.069 g (72% yield) of desired product as a white solid. ES MS m/z 502, 504 (M, M+2).

### Example 174

### N-{[4-Chloro-2-({[(2,6-dimethylphenyl)amino]carbonyl}amino)phenyl]carbonyl}-L-aspartic acid

### Step 1

### Dimethyl N-{[4-chloro-2-({[(2,6-dimethylphenyl)amino]carbonyl}-amino)phenyl]carbonyl}-L-aspartate

HATU (0.268 g, 0.705 mmol) was added to a solution 4-chloro-2-({[(2,6-dimethylphenyl)amino]carbonyl}amino)benzoic acid (0.150 g, 0.47 mmol), dimethyl L-aspartate hydrochloride (0.102 g, 0.52 mmol) and diisopropylethylamine (0.12 mL, 0.705 mmol) in 10 mL of DMF. The mixture was stirred at room temperature overnight, diluted with ethyl acetate and washed with water and brine. The organic phase was dried over anhydrous sodium sulfate and the solvent was removed under vacuum. Chromatography on silica gel with hexane/ethyl acetate gave 0.120 g (55% yield) of desired product as a colorless gum.

### Step 2

### N-{[4-Chloro-2-({[(2,6-dimethylphenyl)amino]carbonyl}amino)phenyl]carbonyl}-L-aspartic acid

Lithium hydroxide (0.062 g, 2.60 mmol) was added to a solution of dimethyl N-{[4-chloro-2-({[(2,6-dimethylphenyl)amino]carbonyl}amino)phenyl]carbonyl}-L-aspartate (0.120 g, 0.26 mmol) in THF:methanol:water/4:1:1. The mixture was stirred at room temperature overnight, acidified with 1N aqueous HCl, and the solvent was removed under vacuum. The residue was extracted between ethyl acetate and water. The organic phase was dried over anhydrous sodium sulfate and the solvent was removed under vacuum to give 0.022 g (20% yield) of desired product as a white solid. ES MS m/z 432 (M−H)

### Example 175

### (2S)-Cyclohexyl({[3-({[(2,6-dimethylphenyl)amino]carbonyl}amino)-4-biphenylyl]carbonyl}amino)ethanoic acid

### Step 1

### Methyl(2S)-cyclohexyl({[3-({[(2,6-dimethylphenyl)amino]carbonyl}-amino)-4-biphenylyl]carbonyl}amino)ethanoate

A mixture of methyl(2S)-({[4-Bromo-2-({[(2,6-dimethylphenyl)amino]carbonyl}-amino)phenyl]carbonyl}amino)(cyclohexyl)ethanoate (0.185 g, 0.36 mmol), phenylboronic acid (0.044 g, 0.36 mmol), transdichlorobis(triphenylphosphine)palladium(II) (0.013 g, 0.018 mmol), and 0.70 mL of 1M aqueous sodium carbonate in 1.5 mL of acetonitrile was heated to 150° C. in a microwave reactor for 5 minutes. The reaction mixture was diluted with ethyl acetate and washed with saturated aqueous sodium bicarbonate. The organic phase was dried over anhydrous sodium sulfate and the solvent was evaporated. Chromatography on silica gel with hexane/ethyl acetate gave 0.116 g (63% yield) of desired product as a white solid.

### Step 2

### (2S)-Cyclohexyl({[3-({[(2,6-dimethylphenyl)amino]carbonyl}amino)-4-biphenylyl]carbonyl}amino)ethanoic acid

Lithium hydroxide (0.054 g, 2.30 mmol) was added to a solution of methyl(2S)-cyclohexyl({[3-({[(2,6-dimethylphenyl)amino]carbonyl}amino)-4-biphenylyl]carbonyl}amino)ethanoate (0.116 g, 0.23 mmol) in THF:methanol:water/4:1:1. The mixture was stirred at room temperature overnight, acidified with 1N aqueous HCl, and the solvent was removed under vacuum. The residue was extracted between ethyl acetate and water. The organic phase was dried over anhydrous sodium sulfate and the solvent was removed under vacuum to give 0.068 g (59% yield) of desired product as a white solid. ES MS m/z 498 (M−H).

### Example 176

### (2S)-cyclohexyl({[2-({[(2,6-dimethylphenyl)amino]carbonyl}amino)-4-methylphenyl]carbonyl}amino)ethanoic acid

### Step 1

### Methyl(2S)-{[(2-amino-4-methylphenyl)carbonyl]amino}(cyclohexyl)ethanoate

HATU (1.13 g, 2.98 mmol) was added to a solution 2-amino-4-methylbenzoic acid (0.300 g, 1.99 mmol), methyl(2S)-cyclohexyl(methylamino)ethanoate hydrochloride (0.495 g, 2.38 mmol) and diisopropylethylamine (0.52 mL, 2.98 mmol) in 20 mL of DMF. The mixture was stirred at room temperature overnight, diluted with ethyl acetate and washed with water and brine. The organic phase was dried over anhydrous sodium sulfate and the solvent was removed under vacuum. Chromatography on silica gel with hexane/ethyl acetate gave 0.270 g (45% yield) of desired product as a colorless gum.

### Step 2

### Methyl(2S)-cyclohexyl({[2-({[(2,6-dimethylphenyl)amino]carbonyl}-amino)-4-methylphenyl]carbonyl}amino)ethanoate

2,6-Dimethylphenylisocyanate (0.27 g, 1.87 mmol) was added to a solution of methyl(2S)-{[(2-amino-4-methylphenyl)carbonyl]amino}(cyclohexyl)ethanoate (0.114 g, 0.37 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent was removed under reduced pressure. Chromatography on silica gel with hexane/ethyl acetate gave 0.153 g (90% yield) of desired product as a white solid.

### Step 3

### 2S)-cyclohexyl({[2-({[(2,6-dimethylphenyl)amino]carbonyl}amino)-4-methylphenyl]carbonyl}amino)ethanoic acid

Lithium hydroxide (0.081 g, 3.40 mmol) was added to a solution of methyl(2S)-cyclohexyl({[2-({[(2,6-dimethylphenyl)amino]carbonyl}amino)-4-methylphenyl]carbonyl}amino)ethanoate (0.153 g, 0.34 mmol) in THF:methanol:water/4:1:1. The mixture was stirred at room temperature overnight, acidified with 1N aqueous HCl, and the solvent was removed under vacuum. The residue was extracted between ethyl acetate and water. The organic phase was dried over anhydrous sodium sulfate and the solvent was removed under vacuum to give 0.092 g (62% yield) of desired product as a white solid. ES MS m/z 436 (M−H).

### Example 177

### (2S)-Cyclohexyl({[4,5-dichloro-2-({[(2,6-dichlorophenyl)amino]-carbonyl}amino)phenyl]carbonyl}amino)ethanoic acid

### Step 1

### 2-Amino-4,5-dichlorobenzoic acid

Azidotrimethylsilane (2.34 g, 20.7 mmol) was added to a suspension of 5,6-dichloro-2-benzofuran-1,3-dione (3.00 g, 13.8 mmol) in 60 mL of toluene. The mixture was heated at 800° C. for 3 hours. The temperature was raised to 100° C. and heating was continued overnight. Toluene was evaporated under reduced pressure and 30 mL of ethanol was added to the residue, and the solvent again removed under vacuum. The resulting white solid was suspended in 50 mL of concentrated HCl and heated to 1000° C. for 1 hour. The mixture was cooled to room temperature and evaporated to dryness to give 3.3 g of an off-white powder. This crude product was carried on to the next step without further purification.

### Step 2

### 1,1-Dimethylethyl (2S)-cyclohexyl({[4,5-dichloro-2-({[(2,6-dichlorophenyl)amino]carbonyl}amino)phenyl]carbonyl}amino)ethanoate

HATU (7.87 g, 20.7 mmol) was added to a solution of 2-amino-4,5-dichlorobenzoic acid (0.300 g, 1.99 mmol), 1,1-dimethylethyl (2S)-amino(cyclohexyl)ethanoate hydrochloride (3.78 g, 15.2 mmol) and diisopropylethylamine (3.6 mL, 20.7 mmol) in 100 mL of DMF. The mixture was stirred at room temperature overnight, then concentrated under vacuum, diluted with ethyl acetate and washed with water and brine. The organic phase was dried over anhydrous sodium sulfate and the solvent was evaporated under vacuum. Chromatography on silica gel with hexane/ethyl acetate gave 2.06 g (37% yield) of desired product as a yellow solid

### Step 3

### Methyl(2S)-cyclohexyl({[4,5-dichloro-2-({[(2,6-dichlorophenyl)amino]carbonyl}amino)phenyl]carbonyl}amino)ethanoate

2,6-Dichlorophenylisocyanate (1.17 g, 6.23 mmol) was added to a solution of 1,1-dimethylethyl (2S)-cyclohexyl({[4,5-dichloro-2-({[(2,6-dichlorophenyl)amino]carbonyl}amino)phenyl]carbonyl}amino)ethanoate (0.500 g, 1.25 mmol) in 20 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent was evaporated under reduced pressure. Chromatography on silica gel with hexane/ethyl acetate gave 0.164 g (22% yield) of desired product as a white solid.

### Step 4

### (2S)-Cyclohexyl({[4,5-dichloro-2-({[(2,6-dichlorophenyl)amino]carbonyl}amino)phenyl]carbonyl}amino)ethanoic acid

Trifluoroacetic acid (0.5 mL, 6.5 mmol) was added to a solution of methyl(2S)-cyclohexyl({[4,5-dichloro-2-({[(2,6-dichlorophenyl)amino]carbonyl}amino)phenyl]carbonyl}amino)ethanoate (0.164 g, 0.28 mmol) in 5 mL of dichloromethane. The mixture was stirred at room temperature overnight. The solvent was evaporated to give 0.155 g (100% yield) of desired product as a white solid. ES MS m/z 531 (M−H).

### Example 178

### (2S)-cyclohexyl({[2-({[(2,6-dimethylphenyl)amino]carbonyl}amino)-4-(trifluoromethyl)phenyl]carbonyl}amino)ethanoic acid

### Step 1

### Methyl(2S)-cyclohexyl({[2-nitro-4-(trifluoromethyl)phenyl]carbonyl}-amino)ethanoate

HATU (0.730 g, 1.92 mmol) was added to a solution 2-nitro-3-trifluoromethylbenzoic acid (0.300 g, 1.28 mmol), (2S)-amino(cyclohexyl)ethanoate hydrochloride (0.265 g, 1.28 mmol) and diisopropylethylamine (0.33 mL, 1.92 mmol) in DMF. The mixture was stirred at room temperature overnight, then diluted with ethyl acetate and washed with water and brine. The organic phase was dried over anhydrous sodium sulfate and the solvent was evaporated under vacuum to give 0.442 g (88% yield) of desired product as a white solid.

### Step 2

### Methyl(2S)-({[2-amino-4-(trifluoromethyl)phenyl]carbonyl}-amino)(cyclohexyl)ethanoate

A mixture of methyl (2S)-cyclohexyl({[2-nitro-4-(trifluoromethyl)phenyl]carbonyl}-amino)ethanoate (0.374 g, 0.96 mmol) and 5% palladium on carbon (0.102 g, 0.048 mmol) in ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.176 g (49% yield) of desired product.

### Step 3

### (2S)-Cyclohexyl({[2-({[(2,6-dimethylphenyl)amino]carbonyl}amino)-4-(trifluoromethyl)phenyl]carbonyl}amino)ethanoic acid

2,6-Methylphenylisocyanate (0.36 g, 2.45 mmol) was added to a solution of methyl(2S)-({[2-amino-4-(trifluoromethyl)phenyl]carbonyl}amino)(cyclohexyl)-ethanoate (0.176 g, 0.49 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.265 g of desired product as a white solid.

### Step 4

### (2S)-cyclohexyl({[2-({[(2,6-dimethylphenyl)amino]carbonyl}amino)-4-(trifluoromethyl)phenyl]carbonyl}amino)ethanoic acid

Lithium hydroxide (0.123 g, 5.1 mmol) was added to a solution of (2S)-cyclohexyl({[2-({[(2,6-dimethylphenyl)amino]carbonyl}amino)-4-(trifluoromethyl)phenyl]carbonyl}amino)ethanoic acid (0.260 g, 0.51 mmol) in THF:methanol:water/4:1:1. The mixture was stirred at room temperature overnight, acidified with 1N aqueous HCl, and the solvent was removed under vacuum. The residue was extracted between ethyl acetate and water. The organic phase was dried over anhydrous sodium sulfate and the solvent was removed under vacuum to give 0.204 g (81% yield) of desired product as a white solid. ES MS m/z 490 (M−H).

### Example 179

### (2S)-({[4-Chloro-2-({[(2,4,6-trimethylphenyl)amino]carbonyl}-amino)phenyl]carbonyl}amino)(cyclohexyl)ethanoic acid

### Step 1

### Methyl(2S)-({[4-chloro-2-({[(2,4,6-trimethylphenyl)amino]carbonyl}-amino)phenyl]carbonyl}amino)(cyclohexyl)ethanoate

2,4,6-Trimethylphenylisocyanate (0.587 g, 3.65 mmol) was added to a solution of methyl(2S)-{[(2-amino-4-chlorophenyl)carbonyl]amino}(cyclohexyl)ethanoate, (0.237 g, 0.73 mmol) in anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent was evaporated under reduced pressure. Chromatography on silica gel with hexane/ethyl acetate gave 0.321 g (90% yield) of desired product as a white solid.

### Step 2

### (2S)-({[4-Chloro-2-({[(2,4,6-trimethylphenyl)amino]carbonyl}amino)phenyl]carbonyl}amino)(cyclohexyl)ethanoic acid

Lithium hydroxide (0.155 g, 6.50 mmol) was added to a solution of methyl(2S)-({[4-chloro-2-({[(2,4,6-trimethylphenyl)amino]carbonyl}amino)phenyl]carbonyl}-amino)(cyclohexyl)ethanoate (0.314 g, 0.65 mmol) in THF:methanol:water/4:1:1. The mixture was stirred at room temperature overnight, acidified with IN aqueous HCl, and the solvent was removed under vacuum. The residue was extracted between ethyl acetate and water. The organic phase was dried over anhydrous sodium sulfate and the solvent was removed under vacuum to give 0.250 g (81% yield) of desired product as a white solid. ES MS m/z 470 (M−H).

### Example 180

### (2S)-Cyclohexyl({[4,5-dichloro-2-({[(2,6-dimethylphenyl)amino]carbonyl}amino)phenyl]carbonyl}amino)ethanoic acid

### Step 1

### 2-Amino-4,5-dichlorobenzoic acid

Azidotrimethylsilane (0.53 g, 6.9 mmol) was added to a suspension of 5,6-dichloro-2-benzofuran-1,3-dione (1.00 g, 4.6 mmol) in 20 mL of toluene. The mixture was heated at 80° C. for 3 hours. The temperature was raised to 100° C. and heating was continued overnight. Toluene was evaporated under reduced pressure and 10 mL of ethanol was added to the residue, and the solvent was again removed under vacuum. The resulting white solid was suspended in 10 mL of concentrated HCl and heated to 100° C. for 1 hour. The mixture was cooled to room temperature and evaporated to dryness to give 0.491 g of an off-white powder. This crude product was carried on to the next step without further purification.

### Step 2

### Methyl(2S)-{[(2-amino-4,5-dichlorophenyl)carbonyl]amino}(cyclohexyl)ethanoate

HATU (1.33 g, 3.49 mmol) was added to a solution 2-amino-4,5-dichlorobenzoic acid (0.480 g, 2.33 mmol), methyl(2S)-amino(cyclohexyl)ethanoate hydrochloride (0.531 g, 2.56 mmol) and diisopropylethylamine (0.61 mL, 3.49 mmol) in 20 mL of DMF. The mixture was stirred at room temperature overnight, then diluted with ethyl acetate and washed with water and brine. The organic phase was dried over anhydrous sodium sulfate and the solvent was evaporated under vacuum. Chromatography on silica gel with hexane/ethyl acetate gave 0.283 g (34% yield) of desired product as a white solid.

### Step 3

### Methyl(2S)-cyclohexyl({[4,5-dichloro-2-({[(2,6-dimethylphenyl)amino]-carbonyl}amino)phenyl]carbonyl}amino)ethanoate

2,6-Dimethylphenylisocyanate (0.34 g, 2.28 mmol) was added to a solution of methyl(2S)-{[(2-amino-4,5-dichlorophenyl)carbonyl]amino}(cyclohexyl)ethanoate (0.164 g, 0.46 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.191 g (82% yield) of desired product as a white solid.

### Step 4

### (2S)-Cyclohexyl({[4,5-dichloro-2-({[(2,6-dimethylphenyl)amino]carbonyl}-amino)phenyl]carbonyl}amino)ethanoic acid

Lithium hydroxide (0.084 g, 3.50 mmol) was added to a solution of methyl(2S)-cyclohexyl({[4,5-dichloro-2-({[(2,6-dimethylphenyl)amino]carbonyl}amino)phenyl]carbonyl}amino)ethanoate (0.178 g, 0.35 mmol) in THF:methanol:water/4:1:1. The mixture was stirred at room temperature overnight, acidified with 1N aqueous HCl, and the solvent was removed under vacuum. The residue was extracted between ethyl acetate and water. The organic phase was dried over anhydrous sodium sulfate and the solvent was removed under vacuum to give 0.141 g (82% yield) of desired product as a white solid. ES MS m/z 490 (M−H).

### Example 181

### (2S)-Cyclohexyl({[2-({[(2,6-dimethylphenyl)amino]carbonyl}amino)-4-(3-pyridinyl)phenyl]carbonyl}amino)ethanoic acid

### Step 1

### Methyl(2S)-({[4-chloro-2-({[(2,6-dimethylphenyl)amino]carbonyl}amino)phenyl]carbonyl}amino)(cyclohexyl)ethanoate

HATU (0.97 g, 2.56 mmol) was added to a solution 4-chloro-2-({[(2,6-dimethylphenyl)amino]carbonyl}amino)benzoic acid (0.546 g, 1.71 mmol), methyl(2S)-amino(cyclohexyl)ethanoate hydrochloride (0.427 g, 2.06 mmol) and diisopropylethylamine (0.44 mL, 2.56 mmol) in DMF. The mixture was stirred at room temperature overnight, then diluted with ethyl acetate and washed with water and brine. The organic phase was dried over anhydrous sodium sulfate and the solvent was evaporated under vacuum. Chromatography on silica gel with hexane/ethyl acetate gave 0.575 g (71% yield) of desired product as a white solid.

### Step 2

### Methyl(2S)-cyclohexyl({[2-({[(2,6-dimethylphenyl)amino]carbonyl}-amino)-4-(3-pyridinyl)phenyl]carbonyl}amino)ethanoate

A mixture of methyl(2S)-({[4-chloro-2-({[(2,6-dimethylphenyl)amino]carbonyl}-amino)phenyl]carbonyl}amino)(cyclohexyl)ethanoate, (0.151 g, 0.32 mmol), 3-pyridinylboronic acid (0.047 g, 0.38 mmol), trans-dichlorobis(tricyclohexylphosphine)palladium(II) (0.012 g, 0.016 mmol), and 2M aqueous sodium carbonate (0.48 mL, 0.96 mmol) in 1.5 mL of acetonitrile was heated in a microwave reactor at 150° C. for 5 minutes. The reaction mixture was cooled to room temperature and diluted with ethyl acetate, washed with saturated aqueous sodium bicarbonate and dried over anhydrous sodium sulfate. The solvent was evaporated and the residue was purified by chromatography on silica gel with hexane/ethyl acetate to give 0.060 g of a white solid containing 70% desired product.

### Step 3

### (2S)-Cyclohexyl({[2-({[(2,6-dimethylphenyl)amino]carbonyl}amino)-4-(3-pyridinyl)phenyl]carbonyl}amino)ethanoic acid

Lithium hydroxide (0.028 g, 1.2 mmol) was added to a solution of methyl(2S)-cyclohexyl({[2-({[(2,6-dimethylphenyl)amino]carbonyl}amino)-4-(3-pyridinyl)phenyl]carbonyl}amino)ethanoate (0.060 g, 0.12 mmol) in THF:methanol:water/2:1:1. The mixture was stirred at room temperature overnight. The solvent was evaporated and 1N aqueous HCl was added to the residue. Aqueous sodium hydroxide was then added to adjust the pH to 5, and the mixture was extracted between ethyl acetate and water. The organic phase was dried over anhydrous sodium sulfate and the solvent was removed under vacuum. The residue was purified by reverse phase HPLC with acetonitrile/water with 0.1% formic acid to give 0.007 g (12% yield) of desired product as a white solid. ES MS m/z 499 (M−H).

### Example 182

### (2S)-Cyclohexyl({[3-({[(2,4,6-trimethylphenyl)amino]carbonyl}-amino)-4-biphenylyl]carbonyl}amino)ethanoic acid

### Step 1

### Methyl 3-nitro-4-biphenylcarboxylate

Methyl 4-chloro-2-nitrobenzoate (0.50 g, 2.32 mmol), phenylboronic acid (0.31 g, 2.55 mmol), trans-dichlorobis(tricyclohexylphosphine)palladium(II) (0.084 g, 0.115 mmol) and cesium fluoride (1.06 g, 6.95 mmol) were mixed in 13 mL of acetonitrile:water/3:1 in each of two microwave reaction vials and heated in a microwave reactor at 150° C. for 5 minutes. The cooled reaction mixtures were combined and filtered through Celite, diluted with ethyl acetate and washed with water and brine. The organic phase was dried over anhydrous sodium sulfate and the solvent was evaporated. Chromatography on silica gel with hexane/ethyl acetate gave 0.95 g (80% yield) of desired product as a yellow oil.

### Step 2

### 3-Nitro-4-biphenycarboxylic acid

Lithium hydroxide (0.259 g, 10.78 mmol) was added to a solution of methyl 3-nitro-4-biphenylcarboxylate (0.924 g, 3.59 mmol) in THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was removed under vacuum to give 0.854 g (98% yield) of the desired acid as a white solid.

### Step 3

### 1,1-Dimethylethyl (2S)-cyclohexyl{[(3-nitro-4-biphenylyl)carbonyl]amino}ethanoate

HATU (1.97 g, 5.17 mmol) was added to a solution of 3-nitro-4-biphenylcarboxylic acid (0.838 g, 3.45 mmol), 1,1-dimethylethyl (2S)-amino(cyclohexyl)ethanoate hydrochloride (0.861 g, 3.45 mmol) and diisopropylethylamine (0.90 mL, 5.17 mmol) in 40 mL of DMF. The mixture was stirred at room temperature overnight, then concentrated under vacuum, diluted with ethyl acetate and washed with water and brine. The organic phase was dried over anhydrous sodium sulfate and the solvent was evaporated. Chromatography on silica gel with hexane/ethyl acetate gave 1.13 g (75% yield) of desired product as a white solid.

### Step 4

### 1,1-Dimethylethyl (2S)-{[(3-amino-4-biphenylyl)carbonyl]amino}-(cyclohexyl)ethanoate

A mixture of 1,1-dimethylethyl (2S)-cyclohexyl{[(3-nitro-4-biphenylyl)carbonyl]amino}ethanoate (1.10 g, 2.51 mmol) and 5% palladium on charcoal (0.267 g, 0.125 mmol), in a pressure reaction vessel, was evacuated and flushed with nitrogen three times, then evacuated and filled with hydrogen and stirred at 50 psi for one hour. Filtration through Celite and evaporation of the solvent gave 0.864 g (84% yield) of desired product as an off-white solid.

### Step 5

### 1,1-Dimethylethyl (2S)-cyclohexyl({[3-({[(2,4,6-trimethylphenyl)amino]-carbonyl}amino)-4-biphenylyl]carbonyl}amino)ethanoate

2,4,6-Trimethylphenylisocyanate (0.598 g, 3.71 mmol) was added to a solution of 1,1-dimethylethyl (2S)-{[(3-amino-4-biphenylyl)carbonyl]amino}(cyclohexyl)ethanoate (0.303 g, 0.74 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent was evaporated under reduced pressure. Chromatography on silica gel with hexane/ethyl acetate gave 0.300 g (71% yield) of desired product as a white solid.

### Step 6

### (2S)-Cyclohexyl({[3-({[(2,4,6-trimethylphenyl)amino]carbonyl}amino)-4-biphenylyl]carbonyl}amino)ethanoic acid

Trifluoroacetic acid (1.5 mL) was added to a solution of 1,1-dimethylethyl (2S)-cyclohexyl({[3-({[(2,4,6-trimethylphenyl)amino]carbonyl}amino)-4-biphenylyl]carbonyl}amino)ethanoate (0.300 g, 0.53 mmol) in 5 mL of dichloromethane. The mixture was stirred at room temperature overnight. The solvent was evaporated and the residue was purified by chromatography on silica gel with hexane/ethyl acetate and triturated with ethyl acetate, to give 0.127 g (47% yield) of desired product as a white solid. ES MS m/z 512 (M−H).

### Example 183

### (2S)-Cyclohexyl({[2-({[(2,6-dimethylphenyl)amino]carbonyl}amino)-4-(2-thienyl)phenyl]carbonyl}amino)ethanoic acid

### Step 1

### Methyl(2S)-{[(2-amino-4-chlorophenyl)carbonyl]amino}(cyclohexyl)ethanoate

HATU (4.56 g, 12.0 mmol) was added to a solution 2-amino-4-chlorobenzoic acid (1.38 g, 8.0 mmol), methyl(2S)-amino(cyclohexyl)ethanoate hydrochloride (2.00 g, 9.6 mmol) and diisopropylethylamine (2.1 mL, 12.0 mmol) in 20 mL of DMF. The mixture was stirred at room temperature overnight, then diluted with ethyl acetate and washed with water and brine. The organic phase was dried over anhydrous sodium sulfate and the solvent was evaporated under vacuum. Chromatography on silica gel with hexane/ethyl acetate gave 1.50 g (58% yield) of desired product as a white solid.

### Step 2

### Methyl(2S)-({[4-chloro-2-({[(2,6-dimethylphenyl)amino]carbonyl}amino)phenyl]carbonyl}amino)(cyclohexyl)ethanoate

2,6-Dimethylphenylisocyanate (3.28 g, 22.6 mmol) was added to a solution of methyl(2S)-{[(2-amino-4-chlorophenyl)carbonyl]amino}(cyclohexyl)ethanoate (1.47 g, 4.53 mmol) in anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent was evaporated under reduced pressure. Chromatography on silica gel with hexane/ethyl acetate gave 1.80 g (84% yield) of desired product as a white solid

### Step 3

### Methyl(2S)-cyclohexyl({[2-({[(2,6-dimethylphenyl)amino]carbonyl}-amino)-4-(2-thienyl)phenyl]carbonyl}amino)ethanoate

A mixture of methyl(2S)-({[4-chloro-2-({[(2,6-dimethylphenyl)amino]carbonyl}-amino)phenyl]carbonyl}amino)(cyclohexyl)ethanoate (0.200 g, 0.42 mmol), 2-thienylboronic acid (0.065 g, 0.51 mmol), trans-dichlorobis(tricyclohexylphosphine)palladium(II) (0.015 g, 0.021 mmol), 0.6 mL of 2M aqueous sodium carbonate and 1.5 mL of acetonitrile was heated in a microwave reactor at 150° C. for 5 minutes. The cooled reaction mixture was diluted with ethyl acetate, washed with saturated aqueous sodium bicarbonate, water and brine, and dried over sodium sulfate. The solvent was evaporated and the residue was purified by chromatography on silica gel with hexane/ethyl acetate to give 0.133 g (61% yield) of desired product as a white solid.

### Step 4

### (2S)-Cyclohexyl({[2-({[(2,6-dimethylphenyl)amino]carbonyl}amino)-4-(2-thienyl)phenyl]carbonyl}amino)ethanoic acid

Lithium hydroxide (0.055 g, 2.3 mmol) was added to a solution of methyl(2S)-cyclohexyl({[2-({[(2,6-dimethylphenyl)amino]carbonyl}amino)-4-(2-thienyl)phenyl]carbonyl}amino)ethanoate (0.119 g, 0.23 mmol) in 5 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated and the residue was purified by chromatography on silica gel with hexane/ethyl acetate to give 0.045 g (39% yield) of the desired product as a white solid. ES MS m/z 504 (M−H).

### Example 184

### (2S)-Cyclohexyl({[2-({[(2,6-dimethylphenyl)amino]carbonyl}amino)-4-(3-thienyl)phenyl]carbonyl}amino)ethanoic acid

### Step 1

### Methyl(2S)-cyclohexyl({[2-({[(2,6-dimethylphenyl)amino]carbonyl}-amino)-4-(3-thienyl)phenyl]carbonyl}amino)ethanoate

A mixture of methyl(2S)-({[4-chloro-2-({[(2,6-dimethylphenyl)amino]carbonyl}-amino)phenyl]carbonyl}amino)(cyclohexyl)ethanoate (0.200 g, 0.42 mmol), 3-thienylboronic acid (0.065 g, 0.51 mmol), trans-dichlorobis(tricyclohexylphosphine)palladium(II) (0.015 g, 0.021 mmol), 0.6 mL of 2M aqueous sodium carbonate and 1.5 mL of acetonitrile was heated in a microwave reactor at 150° C. for 5 minutes. The cooled reaction mixture was diluted with ethyl acetate, washed with saturated aqueous sodium bicarbonate, water and brine, and dried over sodium sulfate. The solvent was evaporated and the residue was purified by chromatography on silica gel with hexane/ethyl acetate to give 0.155 g (71% yield) of desired product as a white solid

### Step 2

### (2S)-Cyclohexyl({[2-({[(2,6-dimethylphenyl)amino]carbonyl}amino)-4-(3-thienyl)phenyl]carbonyl}amino)ethanoic acid

Lithium hydroxide (0.065 g, 2.7 mmol) was added to a solution of methyl(2S)-cyclohexyl({[2-({[(2,6-dimethylphenyl)amino]carbonyl}amino)-4-(3-thienyl)phenyl]carbonyl}amino)ethanoate (0.141 g, 0.27 mmol) in 6 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated and the residue was purified by chromatography on silica gel with hexane/ethyl acetate to give 0.066 g (48% yield) of the desired product as a white solid. ES MS m/z 504 (M−H).

### Example 185

### (2S)-Cyclohexyl({[2-({[(2,6-dimethylphenyl)amino]carbonyl}amino)-4-(4-pyridinyl)phenyl]carbonyl}amino)ethanoic acid

### Step 1

### Methyl(2S)-cyclohexyl({[2-({[(2,6-dimethylphenyl)amino]carbonyl}-amino)-4-(4-pyridinyl)phenyl]carbonyl}amino)ethanoate

A mixture of methyl(2S)-({[4-chloro-2-({[(2,6-dimethylphenyl)amino]carbonyl}-amino)phenyl]carbonyl}amino)(cyclohexyl)ethanoate (0.200 g, 0.42 mmol), 4-pyridinylboronic acid (0.063 g, 0.51 mmol), trans-dichlorobis(tricyclohexylphosphine)palladium(II) (0.015 g, 0.021 mmol), 0.6 mL of 2M aqueous sodium carbonate and 1.5 mL of acetonitrile was heated in a microwave reactor at 150° C. for 5 minutes. The cooled reaction mixture was diluted with ethyl acetate, washed with saturated aqueous sodium bicarbonate, water and brine, and dried over sodium sulfate. The solvent was evaporated and the residue was purified by chromatography on silica gel with hexane/ethyl acetate to give 0.081 g of a white solid containing about 75% of desired product. This material was carried further without additional purification.

### Step 2

### (2S)-Cyclohexyl({[2-({[(2,6-dimethylphenyl)amino]carbonyl}amino)-4-(4-pyridinyl)phenyl]carbonyl}amino)ethanoic acid

Lithium hydroxide (0.035 g, 1.50 mmol) was added to a solution of crude methyl(2S) -cyclohexyl({[2-({[(2,6-dimethylphenyl)amino]carbonyl}amino)-4-(4-pyridinyl)phenyl]carbonyl}amino)ethanoate (0.076 g, approx 0.15 mmol) in 5 mL of THF:methanol:water/4:1:1. The mixture was stirred at room temperature overnight. The solvent was evaporated, and 1N aqueous HCl was added to the residue followed by addition of aqueous sodium hydroxide to a pH of 5. The mixture was extracted with ethyl acetate and the organic layer was dried over sodium sulfate. The solvent was evaporated and the residue was purified by chromatography on silica gel with dichloromethane/methanol to give 0.010 g (13% yield) of the desired product as a white solid. ES MS m/z 501 (M+H).

### Example 186

### (2S)-Cyclohexyl{[(3-{[(2,4,6-trichlorophenyl)acetyl]amino}-4-biphenylyl)carbonyl]amino}ethanoic acid

### Step 1

### Methyl(2S)-{[(4-chloro-2-nitrophenyl)carbonyl]amino}(cyclohexyl)ethanoate

HATU (1.41 g, 3.72 mmol) was added to a solution of 4-chloro-2-nitrobenzoic acid (0.50 g, 2.48 mmol), methyl(2S)-amino(cyclohexyl)ethanoate hydrochloride (0.515 g, 2.48 mmol) and diisopropylethylamine (0.65 mL, 3.72 mmol) in 20 mL of DMF. The mixture was stirred at room temperature for 3.5 hours, then diluted with ethyl acetate and washed with water and brine. The organic phase was dried over anhydrous sodium sulfate and the solvent was evaporated under vacuum. Chromatography on silica gel with hexane/ethyl acetate gave 0.656 g (75% yield) of desired product as a white solid.

### Step 2

### Methyl(2S)-cyclohexyl{[(3-nitro-4-biphenylyl)carbonyl]amino}ethanoate

A mixture of methyl methyl(2S)-{[(4-chloro-2-nitrophenyl)carbonyl]amino}(cyclohexyl)ethanoate (0.294 g, 0.83 mmol), phenylboronic acid (0.121 g, 0.99 mmol), trans-dichlorobis(tricyclohexylphosphine)palladium(II) (0.031 g, 0.041 mmol),1.25 mL of 2M aqueous sodium carbonate and 3.0 mL of acetonitrile was heated in a microwave reactor at 150° C. for 5 minutes. The cooled reaction mixture was diluted with ethyl acetate, washed with saturated aqueous sodium bicarbonate, water and brine, and dried over sodium sulfate. The solvent was evaporated and the residue was purified by chromatography on silica gel with hexane/ethyl acetate to give 0.126 g (38% yield) of desired product as an off-white solid.

### Step 3

### Methyl(2S)-{[(3-amino-4-biphenylyl)carbonyl]amino}(cyclohexyl)ethanoate

5% Palladium on charcoal (0.032 g, 0.015 mmol) was added to a solution of methyl(2S)-cyclohexyl{[(3-nitro-4-biphenylyl)carbonyl]amino}ethanoate (0.121 g, 0.305 mmol), in absolute ethanol. The mixture was evacuated and flushed with nitrogen three times, then evacuated and flushed with hydrogen three times, and stirred at 50 psi for one hour. The reaction mixture was filtered through Celite and the filtrate was evaporated to give 0.116 g of desired product as a yellow solid.

### Step 4

### Methyl(2S)-cyclohexyl{[(3-{[(2,4,6-trichlorophenyl)acetyl]amino}-4-biphenylyl)carbonyl]amino}ethanoate

HATU (0.175 g, 0.46 mmol) was added to a solution of methyl(2S)-{[(3-amino-4-biphenylyl)carbonyl]amino}(cyclohexyl)ethanoate (0.112 g, 0.31 mmol), (2,4,6-trichlorophenyl)acetic acid (0.073 g, 0.31 mmol) and diisopropylethylamine (0.081 mL, 0.46 mmol) in 5 mL of DMF. The mixture was stirred at room temperature overnight. An additional 0.050 g (0.21 mmol) of (2,4,6-trichlorophenyl)acetic acid and 0.100 g (0.26 mmol) of HATU was added and stirring was continued at room temperature for ca. 18 hours. The reaction mixture was diluted with ethyl acetate and washed with water and brine. The organic phase was dried over anhydrous sodium sulfate and the solvent was removed under vacuum. Chromatography on silica gel with hexane/ethyl acetate gave 0.056 g (31% yield) of desired product as a white solid.

### Step 5

### (2S)-Cyclohexyl{[(3-{[(2,4,6-trichlorophenyl)acetyl]amino}-4-biphenylyl)carbonyl]amino}ethanoic acid

Lithium hydroxide (0.022 g, 0.94 mmol) was added to a solution of methyl(2S)-cyclohexyl{[(3-{[(2,4,6-trichlorophenyl)acetyl]amino}-4-biphenylyl)carbonyl]amino}ethanoate (0.055 g, 0.094 mmol) in 1.5 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated to give 0.030 g (56% yield) of the desired product as a white solid. ES MS m/z 573 (M).

### Example 187

### (2S)-Cyclohexyl({[3-({[(2,6-dimethylphenyl)amino]carbonyl}amino)-4′-hydroxy-4-biphenylyl]carbonyl}amino)ethanoic acid

### Step 1

### Methyl(2S)-cyclohexyl({[3-({[(2,6-dimethylphenyl)amino]carbonyl}-amino)-4′-hydroxy-4-biphenylyl]carbonyl}amino)ethanoate

A mixture of methyl(2S)-({[4-chloro-2-({[(2,6-dimethylphenyl)amino]carbonyl}amino)phenyl]carbonyl}amino)(cyclohexyl)ethanoate (0.200 g, 0.42 mmol), 4-hydroxyphenylboronic acid (0.070 g, 0.51 mmol), trans-dichlorobis(tricyclohexylphosphine)palladium(II) (0.015 g, 0.021 mmol), 0.6 mL of 2M aqueous sodium carbonate and 1.5 mL of acetonitrile was heated in a microwave reactor at 150° C. for 5 minutes. The cooled reaction mixture was diluted with ethyl acetate, washed with saturated aqueous sodium bicarbonate, water and brine, and dried over sodium sulfate. The solvent was evaporated and the residue was purified by chromatography on silica gel with hexane/ethyl acetate to give 0.100 g of a white solid containing about 80% of desired product. This material was carried further without additional purification.

### Step 2

### (2S)-Cyclohexyl({[3-({[(2,6-dimethylphenyl)amino]carbonyl}amino)-4′-hydroxy-4-biphenylyl]carbonyl}amino)ethanoic acid

Lithium hydroxide (0.045 g, 1.90 mmol) was added to a solution of methyl(2S)-cyclohexyl({[3-({[(2,6-dimethylphenyl)amino]carbonyl}amino)-4′-hydroxy-4-biphenylyl]carbonyl}amino)ethanoate (0.100 g, approx 0.19 mmol) in 3 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated and the residue was purified by chromatography on silica gel with hexane/ethyl acetate and by reverse phase HPLC with methanol/water with 0.1% formic acid to give 0.035 g (36% yield) of the desired product as a white solid. ES MS m/z 516 (M+H).

### Example 188

### (2S)-Cyclohexyl({[3-({[(2,6-dimethylphenyl)amino]carbonyl}amino)-3′,4′-difluoro-4-biphenylyl]carbonyl}amino)ethanoic acid

### Step 1

### Methyl(2S)-cyclohexyl({[3-({[(2,6-dimethylphenyl)amino]carbonyl}-amino)-3′,4′-difluoro-4-biphenylyl]carbonyl}amino)ethanoate

A mixture of methyl(2S)-({[4-chloro-2-({[(2,6-dimethylphenyl)amino]carbonyl}amino)phenyl]carbonyl}amino)(cyclohexyl)ethanoate (0.200 g, 0.42 mmol), 3,4-difluorophenylboronic acid (0.081 g, 0.51 mmol), trans-dichlorobis(tricyclohexylphosphine)palladium(II) (0.015 g, 0.021 mmol), 0.6 mL of 2M aqueous sodium carbonate and 1.5 mL of acetonitrile was heated in a microwave reactor at 150° C. for 5 minutes. The cooled reaction mixture was diluted with ethyl acetate, washed with saturated aqueous sodium bicarbonate, water and brine, and dried over sodium sulfate. The solvent was evaporated and the residue was purified by chromatography on silica gel with hexane/ethyl acetate to give 0.135 g of a white solid containing about 85% of desired product. This material was carried further without additional purification.

### Step 2

### (2S)-Cyclohexyl({[3-({[(2,6-dimethylphenyl)amino]carbonyl}amino)-3′,4′-difluoro-4-biphenylyl]carbonyl}amino)ethanoic acid

Lithium hydroxide (0.059 g, 2.4 mmol) was added to a solution of methyl(2S)-cyclohexyl({[3-({[(2,6-dimethylphenyl)amino]carbonyl}amino)-3′,4′-difluoro-4-biphenylyl]carbonyl}amino)ethanoate (0.135 g, approx 0.24 mmol) in 3 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated and the residue was purified by reverse phase HPLC with acetonitrile/water with 0.1% formic acid to give 0.037 g (29% yield) of the desired product as a white solid. ES MS m/z 534 (M−H).

### Example 189

### (2S)-Cyclohexyl({[2-({[(2,6-dimethylphenyl)amino]carbonyl}amino)-4-(5-pyrimidinyl)phenyl]carbonyl}amino)ethanoic acid

### Step 1

### Methyl(2S)-cyclohexyl({[2-({[(2,6-dimethylphenyl)amino]carbonyl}-amino)-4-(5-pyrimidinyl)phenyl]carbonyl}amino)ethanoate

A mixture of methyl(2S)-({[4-chloro-2-({[(2,6-dimethylphenyl)amino]carbonyl}amino)phenyl]carbonyl}amino)(cyclohexyl)ethanoate (0.211 g, 0.45 mmol), 5-pyrimidinylboronic acid (0.066 g, 0.54 mmol), trans-dichlorobis(tricyclohexylphosphine)palladium(II) (0.017 g, 0.022 mmol), 0.68 mL of 2M aqueous sodium carbonate and 1.5 mL of acetonitrile was heated in a microwave reactor at 150° C. for 5 minutes. The cooled reaction mixture was diluted with ethyl acetate, washed with saturated aqueous sodium bicarbonate, water and brine, and dried over sodium sulfate. The solvent was evaporated and the residue was purified by chromatography on silica gel with hexane/ethyl acetate to give 0.051 g of a white solid containing about 80% of desired product. This material was carried further without additional purification.

### Step 2

### (2S)-Cyclohexyl({[2-({[(2,6-dimethylphenyl)amino]carbonyl}amino)-4-(5-pyrimidinyl)phenyl]carbonyl}amino)ethanoic acid

Lithium hydroxide (0.024 g, 0.99 mmol) was added to a solution methyl(2S)-cyclohexyl({[2-({[(2,6-dimethylphenyl)amino]carbonyl}amino)-4-(5-pyrimidinyl)phenyl]carbonyl}amino)ethanoate (0.051 g, approx 0.099 mmol) in 3 mL of THF:methanol:water/4:1:1. The mixture was stirred at room temperature overnight. The solvent was evaporated, and 1N aqueous HCl was added to the residue followed by aqueous sodium hydroxide to adjust to pH 5. The mixture was extracted with ethyl acetate and the organic layer was dried over sodium sulfate. The solvent was evaporated and the residue was purified by reverse phase HPLC with acetonitrile/water with 0.1% formic acid to give 0.007 g (14% yield) of the desired product as a white solid. ES MS m/z 500 (M−H).

### Example 190

### (2S)-Cyclohexyl({[2-({[(2,6-dimethylphenyl)amino]carbonyl}amino)-4-fluorophenyl]carbonyl}amino)ethanoic acid

### Step 1

### Methyl(2S)-cyclohexyl{[(4-fluoro-2nitrophenyl)carbonyl]amino}ethanoate

HATU (1.54 g, 4.05 mmol) was added to a solution 4-fluoro-2-nitrobenzoic acid (0.50 g, 2.70 mmol), methyl(2S)-amino(cyclohexyl)ethanoate hydrochloride (0.561 g, 2.70 mmol) and diisopropylethylamine (0.70 mL, 4.05 mmol) in 20 mL of DMF. The mixture was stirred at room temperature overnight. The reaction mixture was diluted with ethyl acetate and washed with water and brine. The organic phase was dried over anhydrous sodium sulfate and the solvent was removed under vacuum to give 0.795 g (87% yield) of desired product as a white solid.

### Step 2

### Methyl(2S)-{[(2-amino-4-fluorophenyl)carbonyl]amino}(cyclohexyl)ethanoate

5% Palladium on charcoal (0.249 g, 0.12 mmol) was added to a solution of methyl(2S)-cyclohexyl{[(4-fluoro-2nitrophenyl)carbonyl]amino}ethanoate (0.791 g, 2.34 mmol) in 20 mL of absolute ethanol. The mixture was evacuated and flushed with nitrogen three times, then evacuated and flushed with hydrogen three times, and stirred at 50 psi for one hour. The reaction mixture was filtered through Celite and the filtrate was evaporated to give 0.600 g (83% yield) of desired product as an off-white solid.

### Step 3

### Methyl(2S)-cyclohexyl({[2-({[(2,6-dimethylphenyl)amino]carbonyl}-amino)-4-fluorophenyl]carbonyl}amino)ethanoate

2,6-Dimethylphenylisocyanate (0.23 g, 1.62 mmol) was added to a solution of methyl(2S)-{[(2-amino-4-fluorophenyl)carbonyl]amino}(cyclohexyl)ethanoate (0.100 g, 0.32 mmol) in anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.103 g (71% yield) of desired product as a white solid.

### Step 4

### (2S)-Cyclohexyl({[2-({[(2,6-dimethylphenyl)amino]carbonyl}amino)-4-fluorophenyl]carbonyl}amino)ethanoic acid

Lithium hydroxide (0.054 g, 2.3 mmol) was added to a solution of methyl(2S)-cyclohexyl({[2-({[(2,6-dimethylphenyl)amino]carbonyl}amino)-4-fluorophenyl]carbonyl}amino)ethanoate (0.103 g, 0.23 mmol) in 5 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated to give 0.070 g (69% yield) of desired product as a white solid. ES MS m/z 440 (M−H).

### Example 191

### (2S)-Cyclohexyl({[3-({[(2,6-dimethylphenyl)amino]carbonyl}amino)-4′-(methyloxy)-4-biphenylyl]carbonyl}amino)ethanoic acid

### Step 1

### Methyl(2S)-cyclohexyl({[3-({[(2,6-dimethylphenyl)amino]carbonyl}-amino)-4′-(methyloxy)-4-biphenylyl]carbonyl}amino)ethanoate

A mixture of methyl(2S)-({[4-chloro-2-({[(2,6-dimethylphenyl)amino]carbonyl}-amino)phenyl]carbonyl}amino)(cyclohexyl)ethanoate (0.195 g, 0.41 mmol), 4-methoxyphenylboronic acid (0.075 g, 0.50 mmol), trans-dichlorobis(tricyclohexylphosphine)palladium(II) (0.015 g, 0.020 mmol), 0.62 mL of 2M aqueous sodium carbonate and 1.5 mL of acetonitrile was heated in a microwave reactor at 150° C. for 5 minutes. The cooled reaction mixture was diluted with ethyl acetate, washed with saturated aqueous sodium bicarbonate, water and brine, and dried over sodium sulfate. The solvent was evaporated and the residue was purified by chromatography on silica gel with hexane/ethyl acetate to give 0.077 g (34% yield) of desired product as a white solid.

### Step 2

### (2S)-Cyclohexyl({[3-({[(2,6-dimethylphenyl)amino]carbonyl}amino)-4′-(methyloxy)-4-biphenylyl]carbonyl}amino)ethanoic acid

Lithium hydroxide (0.034 g, 1.42 mmol) was added to a solution of methyl(2S)-cyclohexyl({[3-({[(2,6-dimethylphenyl)amino]carbonyl}amino)-4′-(methyloxy)-4-biphenylyl]carbonyl}amino)ethanoate (0.077 g, 0.142 mmol) in 3 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated and the residue was purified by chromatography on silica gel with hexane/ethyl acetate to give 0.042 g (56% yield) of desired product as a white solid. ES MS m/z 530 (M+H).

### Example 192

### (2S)-Cyclohexyl({[4-fluoro-2-({[(2,4,6-trimethylphenyl)amino]carbonyl}amino)phenyl]carbonyl}amino)ethanoic acid

### Step 1

### Methyl(2S)-cyclohexyl({[4-fluoro-2-({[(2,4,6-trimethylphenyl)amino]carbonyl}amino)phenyl]carbonyl}amino)ethanoate

2,4,6-Trimethylphenylisocyanate (0.528 g, 3.28 mmol) was added to a solution of methyl(2S)-{[(2-amino-4-fluorophenyl)carbonyl]amino}(cyclohexyl)ethanoate (0.202 g, 0.65 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.272 g (89% yield) of desired product as a white solid.

### Step 2

### (2S)-Cyclohexyl({[4-fluoro-2-({[(2,4,6-trimethylphenyl)amino]carbonyl}amino)phenyl]carbonyl}amino)ethanoic acid

Lithium hydroxide (0.135 g, 5.6 mmol) was added to a solution of methyl(2S)-cyclohexyl({[4-fluoro-2-({[(2,4,6-trimethylphenyl)amino]carbonyl}amino)phenyl]carbonyl}amino)ethanoate (0.264 g, 0.56 mmol) in 3 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated to give 0.181 g (71% yield) of desired product as a white solid. ES MS m/z 456 (M+H).

### Example 193

### (2S)-Cyclohexyl({[4′-(methyloxy)-3-({[(2,4,6-trimethylphenyl)amino]carbonyl}amino)-4-biphenylyl]carbonyl}amino)ethanoic acid

### Step 1

### Methyl(2S)-{[(2-amino-4-chlorophenyl)carbonyl]amino}(cyclohexyl)ethanoate

HATU (16.6 g, 43.6 mmol) was added to a solution 2-amino-4-chlorobenzoic acid (5.00 g, 29.1 mmol), methyl(2S)-amino(cyclohexyl)ethanoate hydrochloride (6.05 g, 29.1 mmol) and diisopropylethylamine (7.6 mL, 43.6 mmol) in DMF. The mixture was stirred at room temperature overnight. The reaction mixture was diluted with ethyl acetate and washed with water and brine. The organic phase was dried over anhydrous sodium sulfate and the solvent was removed under vacuum. The residue was purified by chromatography on silica gel with hexane/ethyl acetate to give 3.31 g (35% yield) of desired product.

### Step 2

### Methyl(2S)-({[4-chloro-2-({[(2,4,6-trimethylphenyl)amino]carbonyl}-amino)phenyl]carbonyl}amino)(cyclohexyl)ethanoate

2,4,6-Trimethylphenylisocyanate (8.13 g, 50.5 mmol) was added to a solution of methyl(2S)-{[(2-amino-4-chlorophenyl)carbonyl]amino}(cyclohexyl)ethanoate (3.28 g, 10.1 mmol) in anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent was evaporated under reduced pressure. Chromatography on silica gel with hexane/ethyl acetate gave 3.70 g (75% yield) of desired product as a white solid.

### Step 3

### Methyl(2S)-cyclohexyl({[4′-(methyloxy)-3-({[(2,4,6-trimethylphenyl)amino]carbonyl}amino)-4-biphenylyl]carbonyl}amino)ethanoate

A mixture methyl(2S)-({[4-chloro-2-({[(2,4,6-trimethylphenyl)amino]carbonyl}amino)phenyl]carbonyl}amino)(cyclohexyl)ethanoate (0.500 g, 1.03 mmol), 4-methoxyphenylboronic acid (0.172 g, 1.13 mmol), trans-dichlorobis(tricyclohexylphosphine)palladium(II) (0.038 g, 0.051 mmol), cesium fluoride (0.469 g, 3.09 mmol), 3 mL of water and 8 mL of acetonitrile was heated in a microwave reactor at 150° C. for 5 minutes. The cooled reaction mixture was filtered through Celite, diluted with ethyl acetate, washed with water, and dried over sodium sulfate. Chromatography on silica gel with hexane/ethyl acetate gave 0.278 g of a white solid containing about 85% desired product.

### Step 4

### (2S)-Cyclohexyl({[4′-(methyloxy)-3-({[(2,4,6-trimethylphenyl)amino]carbonyl}amino)-4-biphenylyl]carbonyl}amino)ethanoic acid

Lithium hydroxide (0.118 g, 5.0 mmol) was added to a solution of methyl(2S)-cyclohexyl({[4′-(methyloxy)-3-({[(2,4,6-trimethylphenyl)amino]carbonyl}amino)-4-biphenylyl]carbonyl}amino)ethanoate (0.278 g, 0.50 mmol) in 9 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated and the residue was purified by chromatography on silica gel with hexane/ethyl acetate to give 0.143 g (53% yield) of desired product as a white solid. ES MS m/z 542 (M−H).

### Example 194

### (2S)-Cyclohexyl({[4′-hydroxy-3-({[(2,4,6-trimethylphenyl)amino]carbonyl}amino)-4-biphenylyl]carbonyl}amino)ethanoic acid

### Step 1

### Methyl(2S)-cyclohexyl({[4′-hydroxy-3-({[(2,4,6-trimethylphenyl)amino]carbonyl}amino)-4-biphenylyl]carbonyl}amino)ethanoate

A mixture of methyl(2S)-({[4-chloro-2-({[(2,4,6-trimethylphenyl)amino]carbonyl}amino)phenyl]carbonyl}amino)(cyclohexyl)ethanoate (0.500 g, 1.03 mmol), 4-hydroxyphenylboronic acid (0.156 g, 1.13 mmol), trans-dichlorobis(tricyclohexylphosphine)palladium(II) (0.038 g, 0.051 mmol), cesium fluoride (0.469 g, 3.09 mmol), 3 mL of water and 8 mL of acetonitrile was heated in a microwave reactor at 150° C. for 5 minutes. The cooled reaction mixture was filtered through Celite, diluted with ethyl acetate, washed with water, and dried over sodium sulfate. Chromatography on silica gel with hexane/ethyl acetate gave 0.181 g (32% yield) of desired product as a white solid.

### Step 2

### Methyl(2S)-cyclohexyl({[4′-hydroxy-3-({[(2,4,6-trimethylphenyl)amino]carbonyl}amino)-4-biphenylyl]carbonyl}amino)ethanoate

Lithium hydroxide (0.080 g, 3.3 mmol) was added to a solution of methyl(2S)-cyclohexyl({[4′-hydroxy-3-({[(2,4,6-trimethylphenyl)amino]carbonyl}amino)-4-biphenylyl]carbonyl}amino)ethanoate (0.181 g, 0.33 mmol) in 6 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated to give 0.130 g (74% yield) of desired product as a white solid. ES MS m/z 530 (M+H).

### Example 195

### (2S)-Cyclohexyl({[4-nitro-2-({[(2,4,6-trimethylphenyl)amino]carbonyl}amino)phenyl]carbonyl}amino)ethanoic acid

### Step 1

### 4-Nitro-2-({[(2,4,6-trimethylphenyl)amino]carbonyl}amino)benzoic acid

2,4,6-Trimethylphenylisocyanate (0.292 g, 1.81 mmol) was added to a mixture of 2-amino-4-nitrobenzoic acid (0.300 g, 1.65 mmol) and triethylamine (0.46 mL, 3.3 mmol) in 10 mL of anhydrous DMF. The mixture was heated to 75° C. for 2 hours. After cooling to room temperature, 2 mL of 6N hydrochloric acid was added and the mixture was diluted with water. The precipitated solid was collected by filtration, washed with water and dried under vacuum to give 0.576 g of a light brown solid containing about 80% of the desired product.

### Step 2

### Methyl(2S)-cyclohexyl({[4-nitro-2-({[(2,4,6-trimethylphenyl)amino]carbonyl}amino)phenyl]carbonyl}amino)ethanoate

HATU (0.929 g, 2.44 mmol) was added to a mixture of 4-Nitro-2-({[(2,4,6-trimethylphenyl)amino]carbonyl}amino)benzoic acid (0.560 g, approx 1.63 mmol), methyl(2S)-amino(cyclohexyl)ethanoate (0.339 g, 1.63 mmol) and diisopropylethylamine (0.42 mL, 2.44 mmol). The mixture was stirred at room temperature for 2.5 hours, diluted with ethyl acetate and washed with water and brine. The organic layer was dried over anhydrous sodium sulfate and the solvent was removed under vacuum. Chromatography on silica gel with hexane/ethyl acetate gave 0.546 g (67% yield) of desired product as a yellow solid.

### Step 3

### (2S)-Cyclohexyl({[4-nitro-2-({[(2,4,6-trimethylphenyl)amino]carbonyl}amino)phenyl]carbonyl}amino)ethanoic acid

Lithium hydroxide (0.048 g, 2.01 mmol) was added to a solution of methyl(2S)-cyclohexyl({[4-nitro-2-({[(2,4,6-trimethylphenyl)amino]carbonyl}amino)phenyl]carbonyl}amino)ethanoate (0.100 g, 0.201 mmol) in 6 mL of THF:methanol:water/4:1:1. The mixture was stirred at room temperature overnight. The solvent was evaporated, and 1N aqueous HCl was added to the residue. The mixture was extracted with ethyl acetate and the organic layer was dried over sodium sulfate. The solvent was evaporated and the residue was purified by chromatography on silica gel with hexane/ethyl acetate to give 0.056 g (58% yield) of desired product as a yellow solid. ES MS m/z 483 (M+H).

### Example 196

### (2S)-({[4-Amino-2-({[(2,4,6-trimethylphenyl)amino]carbonyl}-amino)phenyl]carbonyl}amino)(cyclohexyl)ethanoic acid

### Step 1

### Methyl(2S)-({[4-amino-2-({[(2,4,6-trimethylphenyl)amino]carbonyl}-amino)phenyl]carbonyl}amino)(cyclohexyl)ethanoate

5% Palladium on charcoal (0.085 g, 0.040 mmol) was added to a solution of methyl(2S)-cyclohexyl({[4-nitro-2-({[(2,4,6-trimethylphenyl)amino]carbonyl}amino)phenyl]carbonyl}amino)ethanoate (0.399 g, 0.83 mmol) in 20 mL of absolute ethanol in a pressure vessel. The vessel was evacuated and filled with nitrogen three times, then evacuated and filled with hydrogen three times, and the reaction mixture was stirred at 50 psi for one hour. The reaction mixture was filtered through Celite and the filtrate was evaporated to give 0.284 g (76% yield) of desired product as a light yellow solid.

### Step 2

### (2S)-({[4-Amino-2-({[(2,4,6-trimethylphenyl)amino]carbonyl}amino)phenyl]carbonyl}amino)(cyclohexyl)ethanoic acid

Lithium hydroxide (0.052 g, 2.1 mmol) was added to a solution of methyl(2S)-({[4-amino-2-({[(2,4,6-trimethylphenyl)amino]carbonyl}amino)phenyl]carbonyl}amino)(cyclohexyl)ethanoate (0.100 g, 0.21 mmol) in 6 mL of THF:methanol:water/4:1:1. The mixture was stirred at room temperature overnight. The solvent was evaporated, and 1N aqueous HCl was added to the residue. Aqueous sodium hydroxide was added to adjust the pH to 6. The mixture was extracted with ethyl acetate. An insoluble solid remained between the aqueous and organic layer and was collected by filtration. The organic phase was evaporated. The residue was combined with the solid collected and the mixture was purified by chromatography on silica gel with hexane/ethyl acetate to give 0.033 g (35% yield) of desired product as a yellow solid. ES MS m/z 453 (M+H).

### Example 197

### (2S)-Cyclohexyl({[4′-nitro-3-({[(2,4,6-trimethylphenyl)amino]carbonyl}amino)-4-biphenylyl]carbonyl}amino)ethanoic acid

### Step 1

### 1,1-Dimethylethyl (2S)-{[(2-amino-4-chlorophenyl)carbonyl]amino}(cyclohexyl)ethanoate

HATU (11.42 g, 30.06 mmol) was added to a solution of 2-amino-4-chlorobenzoic acid (3.44 g, 20.04 mmol), 1,1-dimethylethyl (2S)-amino(cyclohexyl)ethanoate hydrochloride (5.00 g, 20.04 mmol) and diisopropylethylamine (5.2 mL, 30.06 mmol) in DMF. The mixture was stirred at room temperature overnight. The reaction mixture was diluted with ethyl acetate and water. The organic phase was washed with water and brine and dried over anhydrous sodium sulfate and the solvent was removed under vacuum. Chromatography on silica gel with hexane/ethyl acetate gave 4.26 g (58% yield of desired product as a white solid.

### Step 2

### 1,1-Dimethylethyl (2S)-({[4-chloro-2-({[(2,4,6-trimethylphenyl)amino]carbonyl}amino)phenyl]carbonyl}amino)(cyclohexyl)ethanoate

2,4,6-Trimethylphenylisocyanate (4.39 g, 27.3 mmol) was added to a solution of 1,1-Dimethylethyl (2S)-{[(2-amino-4-chlorophenyl)carbonyl]amino}(cyclohexyl)ethanoate (2.00 g, 5.45 mmol) in 30 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent was evaporated under reduced pressure. Chromatography on silica gel with hexane/ethyl acetate gave 2.72 g (95% yield) of desired product as a white solid.

### Step 3

### 1,1-Dimethylethyl (2S)-cyclohexyl({[4′-nitro-3-({[(2,4,6-trimethylphenyl)amino]carbonyl}amino)-4-biphenylyl]carbonyl}amino)ethanoate

A mixture of 1,1-dimethylethyl (2S)-({[4-chloro-2-({[(2,4,6-trimethylphenyl)amino]carbonyl}amino)phenyl]carbonyl}amino)(cyclohexyl)ethanoate (0.200 g, 0.38 mmol), 4-nitrophenylboronic acid (0.076 g, 0.45 mmol), trans-dichlorobis(tricyclohexylphosphine)palladium(II) (0.014 g, 0.019 mmol) and 2M aqueous sodium carbonate (0.6 mL) in 1.5 mL of acetonitrile was heated in a microwave reactor at 150° C. for 5 minutes. The cooled reaction mixture was diluted with water and ethyl acetate. The organic phase was dried over sodium sulfate and the solvent was evaporated. The residue was purified by chromatography on silica gel with hexane/ethyl acetate to give 0.174 g of a yellow solid containing about 85% of desired product.

### Step 4

### (2S)-Cyclohexyl({[4′-nitro-3-({[(2,4,6-trimethylphenyl)amino]carbonyl}amino)-4-biphenylyl]carbonyl}amino)ethanoic acid

Trifluoroacetic acid (0.73 mL, 9.47 mmol) was added to a solution of 1,1-dimethylethyl (2S)-cyclohexyl({[4′-nitro-3-({[(2,4,6-trimethylphenyl)amino]carbonyl}amino)-4-biphenylyl]carbonyl}amino)ethanoate (0.174 g, 0.28 mmol) in 5 mL of dichloromethane. The mixture was stirred at room temperature for 48 hours. The solvent was evaporated and the residue was purified by chromatography on silica gel with hexane/ethyl acetate to give 0.117 g (75% yield) of desired product as a yellow solid. ES MS m/z 559 (M+H).

### Example 198

### (2S)-Cyclohexyl({[4′-(hydroxymethyl)-3-({[(2,4,6-trimethylphenyl)amino]carbonyl}amino)-4-biphenylyl]carbonyl}amino)ethanoic acid

### Step 1

### 1,1-Dimethylethyl (2S)-cyclohexyl({[4′-(hydroxymethyl)-3-({[(2,4,6-trimethylphenyl)amino]carbonyl}amino)-4-biphenylyl]carbonyl}amino)ethanoate

A mixture of 1,1-Dimethylethyl (2S)-({[4-chloro-2-({[(2,4,6-trimethylphenyl)amino]carbonyl}amino)phenyl]carbonyl}amino)(cyclohexyl)ethanoate (0.200 g, 0.38 mmol), [4-(hydroxymethyl)phenyl]boronic acid (0.068 g, 0.45 mmol), trans-dichlorobis(tricyclohexylphosphine)palladium(II) (0.014 g, 0.019 mmol) and 2M aqueous sodium carbonate (0.6 mL) in 1.5 mL of acetonitrile was heated in a microwave reactor at 150° C. for 5 minutes. The cooled reaction mixture was diluted with water and ethyl acetate. The organic phase was dried over sodium sulfate and the solvent was evaporated. The residue was purified by chromatography on silica gel with hexane/ethyl acetate to give 0.166 g (73% yield) of desired product as a yellow solid.

### Step 2

### (2S)-Cyclohexyl({[4′-{[(trifluoroacetyl)oxy]methyl}-3-({[(2,4,6-trimethylphenyl)amino]carbonyl}amino)-4-biphenylyl]carbonyl}amino)ethanoic acid

Trifluoroacetic acid (0.73 mL) was added to a solution of 1,1-dimethylethyl (2S)-cyclohexyl({[4′-(hydroxymethyl)-3-({[(2,4,6-trimethylphenyl)amino]carbonyl}amino)-4-biphenylyl]carbonyl}amino)ethanoate (0.166 g, 0.28 mmol) in 5 mL of dichloromethane. The mixture was stirred at room temperature for 48 hours. The solvent was evaporated and the residue was purified by chromatography on silica gel with hexane/ethyl acetate to give 0.123 g of a white solid.

### Step 3

### (2S)-Cyclohexyl({[4′-(hydroxymethyl)-3-({[(2,4,6-trimethylphenyl)amino]carbonyl}amino)-4-biphenylyl]carbonyl}amino)ethanoic acid

Lithium hydroxide (0.025 g, 1.05 mmol) was added to a solution of (2S)-Cyclohexyl({[4′-{[(trifluoroacetyl)oxy]methyl}-3-({[(2,4,6-trimethylphenyl)amino]carbonyl}amino)-4-biphenylyl]carbonyl}amino)ethanoic acid (0.067 g, 0.105 mmol) in 3 mL of THF:methanol:water/4:1:1. The mixture was stirred at room temperature for one hour. The solvent was evaporated, and 1N aqueous HCl was added to the residue. The mixture was extracted with ethyl acetate. The organic phase was evaporated to give 0.050 g (87% yield) of desired product as a white solid. ES MS m/z 542 (M−H).

### Example 199

### (2S)-({[4′-Amino-3-({[(2,4,6-trimethylphenyl)amino]carbonyl}amino)-4-biphenylyl]carbonyl}amino)(cyclohexyl)ethanoic acid

### Step 1

### 1-Dimethylethyl (2S)-({[4′-nitro-3-({[(2,4,6-trimethylphenyl)amino]carbonyl}amino)-4-biphenylyl]carbonyl}amino)(cyclohexyl)ethanoate

A mixture of 1,1-Dimethylethyl (2S)-({[4-chloro-2-({[(2,4,6-trimethylphenyl)amino]carbonyl}amino)phenyl]carbonyl}amino)(cyclohexyl)ethanoate (0.200 g, 0.38 mmol), 4-nitrophenylboronic acid (0.076 g, 0.45 mmol), trans-dichlorobis(tricyclohexylphosphine)palladium(II) (0.014 g, 0.019 mmol) and 2M aqueous sodium carbonate (0.6 mL) in 1.5 mL of acetonitrile was heated in a microwave reactor at 150° C. for 5 minutes. The cooled reaction mixture was diluted with water and ethyl acetate. The organic phase was dried over sodium sulfate and the solvent was evaporated. The residue was purified by chromatography on silica gel with hexane/ethyl acetate to give 0.170 g (73% yield) of desired product as a yellow solid.

### Step 2

### 1,1-Dimethylethyl (2S)-({[4′-amino-3-({[(2,4,6-trimethylphenyl)amino]carbonyl}amino)-4-biphenylyl]carbonyl}amino)(cyclohexyl)ethanoate

5% Palladium on charcoal (0.029 g, 0.013 mmol) was added to a solution of 1,1-dimethylethyl (2S)-cyclohexyl({[4′-nitro-3-({[(2,4,6-trimethylphenyl)amino]carbonyl}amino)-4-biphenylyl]carbonyl}amino)ethanoate (0.166 g, 0.27 mmol) in 10 mL of absolute ethanol in a pressure vessel. The vessel was evacuated and filled with nitrogen three times, then evacuated and filled with hydrogen three times, and stirred at 50 psi for one hour. The reaction mixture was filtered through Celite and the filtrate was evaporated to give 0.108 g (68% yield) of desired product as a white solid.

### Step 3

### (2S)-({[4′-Amino-3-({[(2,4,6-trimethylphenyl)amino]carbonyl}amino)-4-biphenylyl]carbonyl}amino)(cyclohexyl)ethanoic acid

Trifluoroacetic acid (0.5 mL, 6.49 mmol) was added to a solution of 1,1-dimethylethyl (2S)-({[4′-amino-3-({[(2,4,6-trimethylphenyl)amino]carbonyl}amino)-4-biphenylyl]carbonyl}amino)(cyclohexyl)ethanoate (0.105 g, 0.18 mmol) in 4 mL of dichloromethane. The mixture was stirred at room temperature for 18 hours and the solvent was evaporated to give 0.070 g (60% yield) of the trifluoroacetic acid salt of the desired product as a beige solid. ES MS 529 (M+H).

### Example 200

### (2S)-Cyclohexyl({[3-({[(2,4,6-trichlorophenyl)amino]carbonyl}amino)-4-biphenylyl]carbonyl}amino)ethanoic acid

### Step 1

### 3-Nitro-4-biphenylcarboxylic acid

Methyl 4-chloro-2-nitrobenzoate (0.200 g, 0.93 mmol), phenylboronic acid (0.113 g, 0.93 mmol), trans-dichlorobis(tricyclohexylphosphine)palladium(II) (0.034 g, 0.046 mmol) and 2M aqueous sodium carbonate (1.4 mL) were combined in 1 mL of acetonitrile in each of two microwave reaction vials and heated in a microwave reactor at 150° C. for 5 minutes. The cooled reaction mixtures were combined and acidified with concentrated hydrochloric acid and extracted with ethyl acetate. The organic phase was dried over anhydrous sodium sulfate and the solvent was removed under vacuum. The residue was purified by chromatography on silica gel with hexane/ethyl acetate to give 0.283 g (63% yield) of desired product as an off-white solid.

### Step 2

### 1,1-Dimethylethyl (2S)-cyclohexyl{[(3-nitro-4-biphenylyl)carbonyl]amino}ethanoate

HATU (0.644 g, 1.69 mmol) was added to a solution 3-nitro-4-biphenylcarboxylic acid (0.276 g, 1.13 mmol), 1,1-dimethylethyl (2S)-amino(cyclohexyl)ethanoate hydrochloride (0.283 g, 1.13 mmol) and diisopropylethylamine (0.29 mL, 1.69 mmol) in 15 mL of DMF. The mixture was stirred at room temperature overnight. The reaction mixture was extracted between ethyl acetate and water. The organic phase was washed with water and brine and dried over anhydrous sodium sulfate and the solvent was removed under vacuum. Chromatography on silica gel with hexane/ethyl acetate gave 0.355 g of a white solid containing about 80% of the desired product.

### Step 3

### 1,1-Dimethylethyl (2S)-{[(3-amino-4-biphenylyl)carbonyl]amino}(cyclohexyl)ethanoate

5% Palladium on charcoal (0.085 g, 0.040 mmol) was added to a solution of 1,1-dimethylethyl (2S)-cyclohexyl{[(3-nitro-4-biphenylyl)carbonyl]amino}ethanoate (0.350 g, 0.80 mmol) in 20 mL of absolute ethanol in a pressure vessel. The vessel was evacuated and filled with nitrogen three times, then evacuated and filled with hydrogen three times, and stirred at 50 psi for one hour. The reaction mixture was filtered through Celite and the filtrate was evaporated to give 0.310 g (68% yield) of a gray solid containing about 85% of desired product.

### Step 4

### 1,1-Dimethylethyl (2S)-cyclohexyl({[3-({[(2,4,6-trichlorophenyl)amino]carbonyl}amino)-4-biphenylyl]carbonyl}amino)ethanoate

2,4,6-Trichlorophenylisocyanate (0.500 g, 2.25 mmol) was added to a solution of 1,1-dimethylethyl (2S)-{[(3-amino-4-biphenylyl)carbonyl]amino}(cyclohexyl)ethanoate (0.184 g, 0.45 mmol) in 10 mL anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.216 g (76%) of desired product as a yellow solid.

### Step 5

### 2S)-Cyclohexyl({[3-({[(2,4,6-trichlorophenyl)amino]carbonyl}amino)-4-biphenylyl]carbonyl}amino)ethanoic acid

Trifluoroacetic acid (0.5 mL, 6.5 mmol) was added to a solution of 1,1-dimethylethyl (2S)-cyclohexyl({[3-({[(2,4,6-trichlorophenyl)amino]carbonyl}amino)-4-biphenylyl]carbonyl}amino)ethanoate (0.210 g, 0.33 mmol) in 5 mL of dichloromethane. The mixture was stirred at room temperature for ca. 18 hours. The solvent was evaporated and the residue was purified by reverse phase HPLC, on a C18 column with a gradient of acetonitrile/water containing 0.1% formic acid, to give 0.030 g (16% yield) of desired product as a white powder. ES MS m/z 574 (M).

### Example 201

### 3-Methyl-N-{[4′-(methyloxy)-3-({[(2,4,6-trichlorophenyl)amino]carbonyl}amino)-4-biphenylyl]carbonyl}-L-valine

### Step 1

### Methyl 4′-(methyloxy)-3-nitro-4-biphenylcarboxylate

A mixture of methyl 4-chloro-2-nitrobenzoate (0.700 g, 3.25 mmol), 4-methoxyphenylboronic acid (0.494 g, 3.25 mmol), trans-dichlorobis(tricyclohexylphosphine)palladium(II) (0.120 g, 0.16 mmol), 5 mL of 2M aqueous sodium carbonate and 5 mL of acetonitrile, in each of three microwave reaction vials, was heated in a microwave reactor at 150° C. for 5 minutes. After cooling to room temperature, the three reaction mixtures were combined, acidified with concentrated hydrochloric acid and extracted with ethyl acetate. The organic phase was dried over anhydrous sodium sulfate and the solvent was removed under vacuum. The crude product (a mixture of desired product and the corresponding carboxylic acid) was purified by chromatography on silica gel with hexane/ethyl acetate to give 0.96 g (34% yield) of desired product as a yellow solid.

### Step 2

### 4′-(Methyloxy)-3-nitro-4-biphenylcarboxylic acid

Lithium hydroxide (0.238 g, 9.93 mmol) was added to a solution of methyl 4′-(methyloxy)-3-nitro-4-biphenylcarboxylate (0.95 g, 3.31 mmol) in 24 mL of THF:methanol:water/4:1:1. The mixture was stirred at room temperature for 2 hours. The solvent was evaporated and 1N aqueous hydrochloric acid was added to the residue. The resulting suspension was extracted with ethyl acetate, dried over anhydrous sodium sulfate and the solvent removed under vacuum to give 0.854 g (91% yield) of desired product as a yellow solid.

### Step 3

### 3-Methyl-N-{[4′-(methyloxy)-3-nitro-4-biphenylyl]carbonyl}-L-valine HATU (0.627 g, 1.65 mmol) was added to a solution of 4′-(Methyloxy)-3-nitro-4-biphenylcarboxylic acid (0.300 g, 1.10 mmol), methyl 3-methyl-L-valinate hydrochloride (0.199 g, 1.10 mmol), and diisopropylethylamine (0.29 mL, 1.65 mmol) in 15 mL of DMF.

The mixture was stirred at room temperature overnight. The reaction mixture was extracted between ethyl acetate and water. The organic phase was washed with water and brine, dried over anhydrous sodium sulfate and the solvent was removed under vacuum. Chromatography on silica gel with hexane/ethyl acetate gave 0.356 g (81% yield) of desired product as an off-white solid.

### Step 4

### Methyl N-{[3-amino-4′-(methyloxy)-4-biphenylyl]carbonyl}-3-methyl-L-valinate

A mixture of 3-Methyl-N-{[4′-(methyloxy)-3-nitro-4-biphenylyl]carbonyl}-L-valine (0.348 g, 0.87 mmol) and 5% palladium on carbon (0.92 g, 0.043 mmol) in 20 mL of ethanol in a pressure reaction vessel was evacuated and filled with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.300 g (81% yield) of desired

### Step 5

### Methyl 3-methyl-N-{[4′-(methyloxy)-3-({[(2,4,6-trichlorophenyl)amino]carbonyl}amino)-4-biphenylyl]carbonyl}-L-valinate

2,4,6-Trichlorophenylisocyanate (0.394 g, 1.75 mmol) was added to a solution of methyl N-{[3-amino-4′-(methyloxy)-4-biphenylyl]carbonyl}-3-methyl-L-valinate (0.131 g, 0.35 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.091 g (44% yield) of desired product as a white solid.

### Step 6

### 3-Methyl-N-{[4′-(methyloxy)-3-({[(2,4,6-trichlorophenyl)amino]carbonyl}amino)-4-biphenylyl]carbonyl}-L-valine

Lithium hydroxide (0.037 g, 1.5 mmol) was added to a solution of methyl 3-methyl-N-{[4′-(methyloxy)-3-({[(2,4,6-trichlorophenyl)amino]carbonyl}amino)-4-biphenylyl]carbonyl}-L-valinate (0.091 g, 0.15 mmol) in 3 mL of THF:methanol:water/4:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.065 g (75% yield) of desired product as a white solid. ES MS m/z 577 (M−H).

### Example 202

### 3-Methyl-N-{[4′-(methyloxy)-3-({[(2,4,6-trimethylphenyl)amino]carbonyl}amino)-4-biphenylyl]carbonyl}-L-valine

### Step 1

### Methyl 3-methyl-N-{[4′-(methyloxy)-3-({[(2,4,6-trimethylphenyl)amino]carbonyl}amino)-4-biphenylyl]carbonyl}-L-valinate

2,4,6-Trimethylphenylisocyanate (0.344 g, 2.13 mmol) was added to a solution of methyl N-{[3-amino-4′-(methyloxy)-4-biphenylyl]carbonyl}-3-methyl-L-valinate (0.158 g, 0.43 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.191 g (84% yield) of desired product as a white solid.

### Step 2

### 3-Methyl-N-{[4′-(methyloxy)-3-({[(2,4,6-trimethylphenyl)amino]carbonyl}amino)-4-biphenylyl]carbonyl}-L-valine

Lithium hydroxide (0.086 g, 3.60 mmol) was added to a solution of methyl 3-methyl-N-{[4′-(methyloxy)-3-({[(2,4,6-trimethylphenyl)amino]carbonyl}amino)-4-biphenylyl]carbonyl}-L-valinate (0.191 g, 0.36 mmol) in 5 mL of THF:methanol:water/4:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.190 g (100% yield) of desired product as a white solid. ES MS m/z 518 (M+H).

### Example 203

### (2S)-Cyclohexyl({[3-({[(2,6-dichlorophenyl)amino]carbonyl}amino)-4-biphenylyl]carbonyl}amino)ethanoic acid

### Step 1

### 1,1-Dimethylethyl (2S)-cyclohexyl({[3-({[(2,6-dichlorophenyl)amino]carbonyl}amino)-4-biphenylyl]carbonyl}amino)ethanoate

2,6-Dichlorophenylisocyanate (0.276 g, 1.47 mmol) was added to a solution of 1,1-dimethylethyl (2S)-{[(3-amino-4-biphenylyl)carbonyl]amino}(cyclohexyl)ethanoate (0.120 g, 0.29 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.135 g (84% yield) of a white solid containing about 85% of desired product.

### Step 2

### (2S)-Cyclohexyl({[3-({[(2,6-dichlorophenyl)amino]carbonyl}amino)-4-biphenylyl]carbonyl}amino)ethanoic acid

Trifluoroacetic acid (0.5 mL, 6.5 mmol) was added to a solution of 1,1-dimethylethyl (2S)-cyclohexyl({[3-({[(2,6-dichlorophenyl)amino]carbonyl}amino)-4-biphenylyl]carbonyl}amino)ethanoate (0.130 g, 0.22 mmol) in 5 mL of dichloromethane. The mixture was stirred at room temperature for 18 hours and the solvent was removed under vacuum. The residue was triturated with methanol to give 0.030 g (25% yield) of desired product as a white solid. ES MS m/z 538 (M−H).

### Example 204

### (2S)-Cyclohexyl({[4′-[(trifluoromethyl)oxy]-3-({[(2,4,6-trimethylphenyl)amino]carbonyl}amino)-4-biphenylyl]carbonyl}amino)ethanoic acid

### Step 1

### 1,1-Dimethylethyl (2S)-({[4-chloro-2-({[(2,4,6-trimethylphenyl)amino]carbonyl}amino)phenyl]carbonyl}amino)(cyclohexyl)ethanoate

2,4,6-Trimethylphenylisocyanate (4.19 g, 26.0 mmol) was added to a solution of 1,1-dimethylethyl (2S)-{[(2-amino-4-chlorophenyl)carbonyl]amino}(cyclohexyl)ethanoate (1.906 g, 5.20 mmol) in 20 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 2.54 g (92% yield) of desired product as a white solid.

### Step 2

### 1,1-Dimethylethyl (2S)-cyclohexyl({[4′-[(trifluoromethyl)oxy]-3-({[(2,4,6-trimethylphenyl)amino]carbonyl}amino)-4-biphenylyl]carbonyl}amino)ethanoate

A mixture of 1,1-dimethylethyl (2S)-({[4-chloro-2-({[(2,4,6-trimethylphenyl)amino]carbonyl}amino)phenyl]carbonyl}amino)(cyclohexyl)ethanoate (0.150 g, 0.28 mmol), {4-[(trifluoromethyl)oxy]phenyl}boronic acid (0.064 g, 0.31 mmol), trans-dichlorobis(tricyclohexylphosphine)palladium(II) (0.010 g, 0.014 mmol), cesium fluoride (0.128 g, 0.84 mmol), 0.5 mL of water and 1.5 mL of acetonitrile was heated in a microwave reactor at 150° C. for 5 minutes. The cooled reaction mixture was filtered through Celite, diluted with ethyl acetate, washed with water and dried over sodium sulfate. Chromatography on silica gel with hexane/ethyl acetate gave 0.136 g (74% yield) of desired product as a white solid.

### Step 3

### (2S)-Cyclohexyl({[4′-[(trifluoromethyl)oxy]-3-({[(2,4,6-trimethylphenyl)amino]carbonyl}amino)-4-biphenylyl]carbonyl}amino)ethanoic acid

Trifluoroacetic acid (0.5 mL, 6.5 mmol) was added to a solution of 1,1-dimethylethyl (2S)-cyclohexyl({[4′-[(trifluoromethyl)oxy]-3-({[(2,4,6-trimethylphenyl)amino]carbonyl}amino)-4-biphenylyl]carbonyl}amino)ethanoate (0.133 g, 0.203 mmol) in 2 mL of dichloromethane. The mixture was stirred at room temperature for 18 hours and the solvent was removed under vacuum. The residue was purified by reverse phase HPLC on a C18 column with a gradient of acetonitrile/water with 0.1% formic acid to give 0.074 g (61% yield) of desired product as a white powder. ES MS m/z 598 (M+H).

### Example 205

### N—[(S)-cyclohexyl(1H-tetrazol-5-yl)methyl]-4′-(methyloxy)-3-({[(2,4,6-trimethylphenyl)amino]carbonyl}amino)-4-biphenylcarboxamide

### Step 1

### (S)-1-cyclohexyl-1-(1H-tetrazol-5-yl)methanamine

Trifluoroacetic acid (1.5 mL, 19.4 mmol) was added to a solution of 1,1-dimethylethyl[(S)-cyclohexyl(1H-tetrazol-5-yl)methyl]carbamate (0.500 g, 1.78 mmol) in dichloromethane. The mixture was stirred at room temperature for 3 hours and the solvent was removed under vacuum to give a yellow oil. The crude product was taken on to the next step without further purification.

### Step 2

### N—[(S)-Cyclohexyl(1H-tetrazol-5-yl)methyl]-4′-(methyloxy)-3-nitro-4-biphenylcarboxamide

HATU (0.519 g, 0.47 mmol) was added to a solution of (4′-(methyloxy)-3-nitro-4-biphenylcarboxylic acid (0.300, 1.09 mmol), (S)-1-cyclohexyl-1-(1H-tetrazol-5-yl)methanamine (approx. 1.7 mmol) and diisopropylethylamine (0.24 mL, 1.37 mmol) in 20 mL of DMF. The mixture was stirred at room temperature overnight. DMF was evaporated under vacuum and the residue was extracted between ethyl acetate and water. The organic phase was washed with water and brine, dried over anhydrous sodium sulfate and the solvent was removed under vacuum. Chromatography on silica gel with hexane/ethyl acetate gave 0.168 g (35% yield) of desired product as a white solid.

### Step 3

### 3-Amino-N—[(S)-cyclohexyl(1H-tetrazol-5-yl)methyl]-4′-(methyloxy)-4-biphenylcarboxamide

A mixture of N—[(S)-cyclohexyl(1H-tetrazol-5-yl)methyl]-4′-(methyloxy)-3-nitro-4-biphenylcarboxamide (0.165 g, 0.38 mmol) and 5% palladium on carbon (0.040 g, 0.019 mmol) in 30 mL of ethanol in a pressure reaction vessel was evacuated and filled with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.145 g of a yellow solid containing mainly the desired product.

### Step 4

### N—[(S)-cyclohexyl(1H-tetrazol-5-yl)methyl]-4′-(methyloxy)-3-({[(2,4,6-trimethylphenyl)amino]carbonyl}amino)-4-biphenylcarboxamide

2,4,6-Trimethylphenylisocyanate (0.287 g, 1.78 mmol) was added to a solution of 3-Amino-N—[(S)-cyclohexyl(1H-tetrazol-5-yl)methyl]-4′-(methyloxy)-4-biphenylcarboxamide (0.145 g, 0.36 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. The residue was purified by chromatography on silica gel with hexane/ethyl acetate and reverse phase HPLC on C18 with acetonitrile/water containing 0.1% formic acid to give 0.015 g (7% yield) of desired product as a white solid. ES MS m/z 566 (M−H).

### Example 206

### 2S)-Cyclohexyl({[4-{[(methylamino)carbonyl]amino}-2-({[(2,4,6-trimethylphenyl)amino]carbonyl}amino)phenyl]carbonyl}amino)ethanoic acid

### Step 1

### 4-Nitro-2-({[(2,4,6-trimethylphenyl)amino]carbonyl}amino)benzoic acid

2,4,6-Trimethylphenylisocyanate (2.92 g, 18.1 mmol) was added to a mixture of 2-amino-4-nitrobenzoic acid (3.00 g, 16.5 mmol) and triethylamine (4.6 mL, 33.0 mmol) in 100 mL of anhydrous DMF. The mixture was heated to 75° C. for 2 hours. After cooling to room temperature, 20 mL of 6N hydrochloric acid was added and the mixture was diluted with water. The precipitated solid was collected by filtration, washed with water and dried under vacuum to give 5.97 g of a yellow solid. This crude product was carried further without additional purification.

### Step 2

### 1,1-Dimethylethyl (2S)-cyclohexyl({[4-nitro-2-({[(2,4,6-trimethylphenyl)amino]carbonyl}amino)phenyl]carbonyl}amino)ethanoate

HATU (9.40 g, 24.75 mmol) was added to a solution of 4-Nitro-2-({[(2,4,6-trimethylphenyl)amino]carbonyl}amino)benzoic acid (5.66 g, 16.5 mmol), 1,1-dimethylethyl (2S)-amino(cyclohexyl)ethanoate hydrochloride (4.12 g, 16.5 mmol) and diisopropylethylamine (6.4 mL, 24.75 mmol) in 200 mL of DMF. The mixture was stirred at room temperature overnight. The reaction mixture was diluted with ethyl acetate and washed with water and brine. The organic phase was dried over anhydrous sodium sulfate and the solvent was removed under vacuum. Chromatography on silica gel with hexane/ethyl acetate gave 5.97 g (67% yield) of desired product as a light yellow solid.

### Step 3

### 1,1-Dimethylethyl (2S)-({[4-amino-2-({[(2,4,6-trimethylphenyl)amino]carbonyl}amino)phenyl]carbonyl}amino)(cyclohexyl)ethanoate

A mixture of 1,1-dimethylethyl (2S)-cyclohexyl({[4-nitro-2-({[(2,4,6-trimethylphenyl)amino]carbonyl}amino)phenyl]carbonyl}amino)ethanoate (3.00 g, 5.58 mmol) and 5% palladium on carbon (0.59 g, 0.28 mmol) in 150 mL of ethanol in a pressure reaction vessel was evacuated and filled with nitrogen three times, then evacuated and filled with 50 psi of hydrogen and stirred for one hour. The reaction vessel was then evacuated and flushed with nitrogen. The mixture was filtered through Celite and the filtrate was evaporated to give 2.49 g (84% yield) of desired product as a light yellow solid

### Step 4

### 1,1-Dimethylethyl (2S)-cyclohexyl({[4-{[(methylamino)carbonyl]amino}-2-({[(2,4,6-trimethylphenyl)amino]carbonyl}amino)phenyl]carbonyl}amino)ethanoate

Methylisocyanate (0.084 g, 1.48 mmol) was added to a solution of 1,1-dimethylethyl (2S)-({[4-amino-2-({[(2,4,6-trimethylphenyl)amino]carbonyl}amino)phenyl]carbonyl}amino)(cyclohexyl)ethanoate (0.150 g, 0.29 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.134 g (82% yield) of desired product as a white solid.

### Step 5

### 2S)-Cyclohexyl({[4-{[(methylamino)carbonyl]amino}-2-({[(2,4,6-trimethylphenyl)amino]carbonyl}amino)phenyl]carbonyl}amino)ethanoic acid

Trifluoracetic acid (0.5 mL, 6.49 mmol) was added to a solution of 1-dimethylethyl (2S)-cyclohexyl({[4-{[(methylamino)carbonyl]amino}-2-({[(2,4,6-trimethylphenyl)amino]carbonyl}amino)phenyl]carbonyl}amino)ethanoate (0.132 g, 0.23 mmol) in 2 mL of dichloromethane. The mixture was stirred at room temperature overnight and the solvent was evaporated. The residue was purified by reverse phase HPLC on a C18 column with a water/acetonitrile gradient with 0.1% formic acid to give 0.052 g (44% yield) of desired product as a white solid. ES MS m/z 510 (M+H).

### Example 207

### (2S)-Cyclohexyl({[4-(dibutylamino)-2-({[(2,4,6-trimethylphenyl)amino]carbonyl}amino)phenyl]carbonyl}amino)ethanoic acid

### Step 1

### 1,1-Dimethylethyl (2S)-cyclohexyl({[4-(dibutylamino)-2-({[(2,4,6-trimethylphenyl)amino]carbonyl}amino)phenyl]carbonyl}amino)ethanoate

Butyraldehyde (0.021 g, 0.29 mmol) was added to a solution of 1,1-dimethylethyl (2S)-({[4-amino-2-({[(2,4,6-trimethylphenyl)amino]carbonyl}amino)phenyl]carbonyl}amino)(cyclohexyl)ethanoate (0.150 g, 0.29 mmol) in 5 mL of 1,2-dichloroethane. Sodium triacetoxyborohydride (0.154 g, 0.725 mmol) was added after a few minutes and the mixture was stirred at room temperature for ca. 18 hours. The reaction mixture was diluted with ethyl acetate, washed with saturated aqueous sodium bicarbonate and dried over sodium sulfate. The solvent was evaporated and the residue was purified by chromatography on silica gel with hexane/ethyl acetate to give 0.101 g of product as a white solid.

### Step 2

### (2S)-Cyclohexyl({[4-(dibutylamino)-2-({[(2,4,6-trimethylphenyl)amino]carbonyl}amino)phenyl]carbonyl}amino)ethanoic acid

Trifluoroacetic acid (0.5 mL, 6.49 mmol) was added to a solution of 1,1-dimethylethyl (2S)-cyclohexyl({[4-(dibutylamino)-2-({[(2,4,6-trimethylphenyl)amino]carbonyl}amino)phenyl]carbonyl}amino)ethanoate (0.101 g, 0.18 mmol) in 5 mL of dichloromethane. The mixture was stirred at room temperature overnight and the solvent was evaporated. The residue was purified by chromatography on silica gel with hexane/ethyl acetate to give 0.077 g (63% yield) of the trifluoroacetic acid salt of the desired product as a white solid. ES MS m/z 565 (M+H).

### Example 208

### (2S)-Cyclohexyl{[(2-{[({2,6-dichloro-4-[(trifluoromethyl)oxy]phenyl}amino)carbonyl]amino}-4-fluorophenyl)carbonyl]amino}ethanoic acid

### Step 1

### Methyl(2S)-cyclohexyl{[(2-{[({2,6-dichloro-4-[(trifluoromethyl)oxy]phenyl}amino)carbonyl]amino}-4-fluorophenyl)carbonyl]amino}ethanoate

1,3-Dichloro-2-isocyanato-5-[(trifluoromethyl)oxy]benzene (0.177 g, 0.65 mmol) was added to a solution of 1 methyl(2S)-{[(2-amino-4-fluorophenyl)carbonyl]amino}(cyclohexyl)ethanoate (0.100 g, 0.325 mmol) in anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.163 g (86% yield) of desired product as a white solid.

### Step 2

### (2S)-Cyclohexyl{[(2-{[({2,6-dichloro-4-[(trifluoromethyl)oxy]phenyl}amino)carbonyl]amino}-4-fluorophenyl)carbonyl]amino}ethanoic acid

Lithium hydroxide (0.065 g, 2.70 mmol) was added to a solution of methyl(2S)-cyclohexyl{[(2-{[({2,6-dichloro-4-[(trifluoromethyl)oxy]phenyl}amino)carbonyl]amino}-4-fluorophenyl)carbonyl]amino}ethanoate (0.157 g, 0.27 mmol) in THF:methanol:water/4:1:1. The mixture was stirred at room temperature for ca. 18 hours. The solvent was evaporated, 1N aqueous hydrochloric acid was added, and the resulting suspension was extracted with ethyl acetate. The organic phase was dried over sodium sulfate and the solvent was evaporated. Chromatography on silica gel with hexane/ethyl acetate gave 0.065 g (45% yield) of desired product as a white solid. ES MS m/z 564 (M−H).

### Example 209

### (2S)-Cyclohexyl({[3′,4′-difluoro-3-({[(2,4,6-trimethylphenyl)amino]carbonyl}amino)-4-biphenylyl]carbonyl}amino)ethanoic acid

### Step 1

### Methyl 3′,4′-difluoro-3-nitro-4-biphenylcarboxylate

In each of two microwave reaction vials, a mixture of methyl 4-chloro-2-nitrobenzoate (0.500 g, 2.62 mmol), 3,4-difluorophenylboronic acid (0.403 g, 2.55 mmol), trans-dichlorobis(tricyclohexylphosphine)palladium(II) (0.086 g, 0.115 mmol), cesium fluoride (1.05 g, 6.95 mmol), 2.5 mL of water and 7.5 mL of acetonitrile was heated in a microwave reactor at 150° C. for 5 minutes. The cooled reaction mixture was diluted with ethyl acetate, washed with water and dried over sodium sulfate. Chromatography on silica gel with hexane/ethyl acetate gave 0.916 g (67% yield) of desired product as a white solid.

### Step 2

### 3′,4′-Difluoro-3-nitro-4-biphenylcarboxylic acid

Lithium hydroxide (0.219 g, 9.13 mmol) was added to a solution of methyl 3′,4′-difluoro-3-nitro-4-biphenylcarboxylate (0.892 g, 3.04 mmol) in THF:methanol:water/3:1:1. The mixture was stirred at room temperature for ca. 18 hours. The solvent was evaporated, 1N aqueous hydrochloric acid was added, and the resulting suspension was extracted with ethyl acetate. The organic phase was dried over sodium sulfate and the solvent evaporated to give 0.810 g (95% yield) of desired product as a white solid.

### Step 3

### 1,1-Dimethylethyl (2S)-cyclohexyl{[(3′,4′-difluoro-3-nitro-4-biphenylyl)carbonyl]amino}ethanoate

HATU (1.12 g, 2.95 mmol) was added to a solution of 3′,4′-Difluoro-3-nitro-4-biphenylcarboxylic acid (0.550 g, 1.97 mmol), 1,1-dimethylethyl (2S)-amino(cyclohexyl)ethanoate hydrochloride (0.541 g, 2.17 mmol) and diisopropylethylamine (0.52 mL, 2.95 mmol) in 20 mL of DMF. The mixture was stirred at room temperature overnight. The reaction mixture was extracted between ethyl acetate and water. The organic phase was washed with water and brine and dried over anhydrous sodium sulfate and the solvent was removed under vacuum. Chromatography on silica gel with hexane/ethyl acetate gave 0.739 g (79% yield) of desired product as a white solid.

### Step 4

### 1,1-Dimethylethyl (2S)-{[(3-amino-3′,4′-difluoro-4-biphenylyl)carbonyl]amino}(cyclohexyl)ethanoate

A mixture of 1,1-dimethylethyl (2S)-cyclohexyl{[(3′,4′-difluoro-3-nitro-4-biphenylyl)carbonyl]amino}ethanoate (0.711 g, 1.50 mmol) and 5% palladium on carbon (0.160 g, 0.075 mmol) in 25 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.652 g (97% yield) of desired product as a beige solid.

### Step 5

### 1,1-Dimethylethyl (2S)-cyclohexyl({[3′,4′-difluoro-3-({[(2,4,6-trimethylphenyl)amino]carbonyl}amino)-4-biphenylyl]carbonyl}amino)ethanoate

2,4,6-Trimethylphenylisocyanate (0.362 g, 2.25 mmol) was added to a solution of 1,1-dimethylethyl (2S)-{[(3-amino-3′,4′-difluoro-4-biphenylyl)carbonyl]amino}(cyclohexyl)ethanoate (0.200 g, 0.45 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.249 g of a white solid containing mainly desired product and some unknown impurity. This material was carried on to the next step without further purification.

### Step 6

### (2S)-Cyclohexyl({[3′,4′-difluoro-3-({[(2,4,6-trimethylphenyl)amino]carbonyl}amino)-4-biphenylyl]carbonyl}amino)ethanoic acid

Trifluoroacetic acid (0.5 mL, 6.49 mmol) was added to a solution of 1,1-dimethylethyl (2S)-cyclohexyl({[3′,4′-difluoro-3-({[(2,4,6-trimethylphenyl)amino]carbonyl}amino)-4-biphenylyl]carbonyl}amino)ethanoate (0.239 g, 0.39 mmol) in 5 mL of dichloromethane. The mixture was stirred at room temperature overnight. The solvent was evaporated and the residue was purified by chromatography on silica gel with hexane/ethyl acetate to give 0.173 g (81% yield) of desired product as a white solid. ES MS m/z 550 (M+H).

### Example 210

### (2S)-Cyclopentyl({[4′-(methyloxy)-3-({[(2,4,6-trimethylphenyl)amino]carbonyl}amino)-4-biphenylyl]carbonyl}amino)ethanoic acid

### Step 1

### Methyl(2S)-cyclopentyl({[4′-(methyloxy)-3-nitro-4-biphenylyl]carbonyl}amino)ethanoate

HATU (0.515 g, 1.36 mmol) was added to a solution of 4′-(methyloxy)-3-nitro-4-biphenylcarboxylic acid (0.247 g, 0.904 mmol), methyl(2S)-amino(cyclopentyl)ethanoate trifluoroacetate (0.245 g, 0.904 mmol) and diisopropylethylamine (0.24 mL, 1.36 mmol) in 10 mL of DMF. The mixture was stirred at room temperature overnight, then diluted with ethyl acetate and washed with water and brine. The organic phase was dried over anhydrous sodium sulfate and the solvent was removed under vacuum. Chromatography on silica gel with hexane/ethyl acetate gave 0.235 g (63% yield) of desired product as a white solid.

### Step 2

### Methyl(2S)-({[3-amino-4′-(methyloxy)-4-biphenylyl]carbonyl}amino)(cyclopentyl)ethanoate

A mixture of methyl(2S)-cyclopentyl({[4′-(methyloxy)-3-nitro-4-biphenylyl]carbonyl}amino)ethanoate (0.201 g, 0.49 mmol) and 5% palladium on carbon (0.052 g, 0.024 mmol) in 15 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.177 g (94% yield) of desired product as a white solid.

### Step 3

### Methyl(2S)-cyclopentyl({[4′-(methyloxy)-3-({[(2,4,6-trimethylphenyl)amino]carbonyl}amino)-4-biphenylyl]carbonyl}amino)ethanoate

2,4,6-Trimethylphenylisocyanate (0.339 g, 2.11 mmol) was added to a solution of methyl(2S)-({[3-amino-4′-(methyloxy)-4-biphenylyl]carbonyl}amino)(cyclopentyl)ethanoate (0.161 g, 0.42 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.116 g of a white solid containing 85-90% desired product. This material was carried on to the next step without further purification.

### Step 4

### (2S)-Cyclopentyl({[4′-(methyloxy)-3-({[(2,4,6-trimethylphenyl)amino]carbonyl}amino)-4-biphenylyl]carbonyl}amino)ethanoic acid

Lithium hydroxide (0.050 g, 2.08 mmol) was added to a solution of methyl(2S)-cyclopentyl({[4′-(methyloxy)-3-({[(2,4,6-trimethylphenyl)amino]carbonyl}amino)-4-biphenylyl]carbonyl}amino)ethanoate (0.113 g, 0.21 mmol) in 3 mL of THF:methanol:water/4:1:1. The mixture was stirred at room temperature for 2 hours and 1N aqueous HCl was added. The solvents were evaporated and the residue was extracted between ethyl acetate and water. The organic phase was dried over sodium sulfate and the solvent was evaporated to give 0.066 g (59% yield) of desired product as a white solid. ES MS m/z 528 (M−H).

### Example 211

### (2S)-Cyclopentyl({[4-fluoro-2-({[(2,4,6-trimethylphenyl)amino]carbonyl}amino)phenyl]carbonyl}amino)ethanoic acid

### Step 1

### Methyl(2S)-cyclopentyl{[(4-fluoro-2-nitrophenyl)carbonyl]amino}ethanoate

HATU (1.54 g, 4.05 mmol) was added to a solution of 4-fluoro-2-nitrobenzoic acid (0.500 g, 2.70 mmol), methyl(2S)-amino(cyclopentyl)ethanoate trifluoroacetate (0.732 g, 2.70 mmol) and diisopropylethylamine (0.70 mL, 4.05 mmol) in DMF. The mixture was stirred at room temperature overnight, then diluted with ethyl acetate and washed with water and brine. The organic phase was dried over anhydrous sodium sulfate and the solvent was removed under vacuum. Chromatography on silica gel with hexane/ethyl acetate gave 0.519 g (59% yield) of desired product as a white solid.

### Step 2

### Methyl(2S)-{[(2-amino-4-fluorophenyl)carbonyl]amino}(cyclopentyl)ethanoate

A mixture of methyl(2S)-cyclopentyl{[(4-fluoro-2-nitrophenyl)carbonyl]amino}ethanoate (0.473 g, 1.46 mmol) and 5% palladium on carbon (0.155 g, 0.073 mmol) in 25 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.360 g (84% yield) of desired product as a white solid.

### Step 3

### Methyl(2S)-cyclopentyl({[4-fluoro-2-({[(2,4,6-trimethylphenyl)amino]carbonyl}amino)phenyl]carbonyl}amino)ethanoate

2,4,6-Trimethylphenylisocyanate (0.548 g, 3.40 mmol) was added to a solution of methyl(2S)-{[(2-amino-4-fluorophenyl)carbonyl]amino}(cyclopentyl)ethanoate (0.200 g, 0.68 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.266 g (85% yield) of desired product as a white solid.

### Step 4

### (2S)-Cyclopentyl({[4-fluoro-2-({[(2,4,6-trimethylphenyl)amino]carbonyl}amino)phenyl]carbonyl}amino)ethanoic acid

Lithium hydroxide (0.128 g, 5.31 mmol) was added to a solution of methyl(2S)-cyclopentyl({[4-fluoro-2-({[(2,4,6-trimethylphenyl)amino]carbonyl}amino)phenyl]carbonyl}amino)ethanoate (0.242 g, 0.53 mmol) in 6 mL of THF:methanol:water/4:1:1. The mixture was stirred at room temperature for 2 hours and 1N aqueous HCl was added. The solvents were evaporated and the residue was extracted between ethyl acetate and water. The organic phase was dried over sodium sulfate and the solvent was evaporated to give 0.201 g (86% yield) of desired product as a white solid. ES MS m/z 440 (M−H).

### Example 212

### (2S)-Cyclohexyl{[(3-{[({2,6-dichloro-4-[(trifluoromethyl)oxy]phenyl}amino)carbonyl]amino}-3′,4′-difluoro-4-biphenylyl)carbonyl]amino}ethanoic acid

### Step 1

### 1,1-Dimethylethyl (2S)-cyclohexyl{[(3-{[({2,6-dichloro-4-[(trifluoromethyl)oxy]phenyl}amino)carbonyl]amino}-3′,4′-difluoro-4-biphenylyl)carbonyl]amino}ethanoate

1,3-Dichloro-2-isocyanato-5-[(trifluoromethyl)oxy]benzene (0.177 g, 0.65 mmol) was added to a solution of 1,1-dimethylethyl (2S)-{[(3-amino-3′,4′-difluoro-4-biphenylyl)carbonyl]amino}(cyclohexyl)ethanoate (0.150 g, 0.29 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.229 g (59% yield) of desired product as a white solid.

### Step 2

### (2S)-Cyclohexyl{[(3-{[({2,6-dichloro-4-[(trifluoromethyl)oxy]phenyl}amino)carbonyl]amino}-3′,4′-difluoro-4-biphenylyl)carbonyl]amino}ethanoic acid

Trifluoroacetic acid (0.5 mL, 6.5 mmol) was added to a solution 1,1-dimethylethyl (2S)-cyclohexyl{[(3-{[({2,6-dichloro-4-[(trifluoromethyl)oxy]phenyl}amino)carbonyl]amino}-3′,4′-difluoro-4-biphenylyl)carbonyl]amino}ethanoate (0.222 g, 0.31 mmol) in 5 mL of dichloromethane. The mixture was stirred at room temperature overnight. The solvent was evaporated and the residue was purified by chromatography on silica gel with hexane/ethyl acetate to give 0.155 g (76% yield) of desired product as a white solid. ES MS m/z 658 (M−H).

### Example 213

### (2S)-Cyclohexyl({[4′-[(dimethylamino)methyl]-3-({[(2,4,6-trimethylphenyl)amino]carbonyl}amino)-4-biphenylyl]carbonyl}amino)ethanoic acid

### Step 1

### 1,1-Dimethylethyl (2S)-cyclohexyl({[4′-(hydroxymethyl)-3-({[(2,4,6-trimethylphenyl)amino]carbonyl}amino)-4-biphenylyl]carbonyl}amino)ethanoate

In each of two microwave reaction vials, a mixture of 1,1-Dimethylethyl (2S)-({[4-chloro-2-({[(2,4,6-trimethylphenyl)amino]carbonyl}amino)phenyl]carbonyl}amino)(cyclohexyl)ethanoate (0.500 g, 0.94 mmol), [4-(hydroxymethyl)phenyl]boronic acid (0.158 g, 1.04 mmol), trans-dichlorobis(tricyclohexylphosphine)palladium(II) (0.035 g, 0.047 mmol), cesium fluoride (0.43 g, 2.83 mmol), 2.5 mL of water and 7.5 mL of acetonitrile was heated in a microwave reactor at 150° C. for 5 minutes. The cooled reaction mixtures were combined, filtered through Celite, diluted with ethyl acetate, washed with water and dried over sodium sulfate. Chromatography on silica gel with hexane/ethyl acetate gave 0.583 g of a white solid containing about 85% of desired product. This product was carried on to the next step without further purification.

### Step 2

### 1,1-Dimethylethyl (2S)-cyclohexyl({[4′-formyl-3-({[(2,4,6-trimethylphenyl)amino]carbonyl}amino)-4-biphenylyl]carbonyl}amino)ethanoate

Manganese dioxide (1.67 g, 19.3 mmol) was added to a solution of 1,1-dimethylethyl (2S)-cyclohexyl({[4′-(hydroxymethyl)-3-({[(2,4,6-trimethylphenyl)amino]carbonyl}amino)-4-biphenylyl]carbonyl}amino)ethanoate (0.577 g, 0.96 mmol) in 50 mL of dichloromethane. The mixture was stirred at room temperature for 18 hours, filtered through Celite and the solvent evaporated. Chromatography on silica gel gave 0.446 g of a white solid containing 90% desired product.

### Step 3

### 1,1-Dimethylethyl (2S)-cyclohexyl({[4′-[(dimethylamino)methyl]-3-({[(2,4,6-trimethylphenyl)amino]carbonyl}amino)-4-biphenylyl]carbonyl}amino)ethanoate

Dimethylamine (0.85 mL of a 2M solution in THF) was added to a solution of 1,1-dimethylethyl (2S)-cyclohexyl({[4′-formyl-3-({[(2,4,6-trimethylphenyl)amino]carbonyl}amino)-4-biphenylyl]carbonyl}amino)ethanoate (0.206 g, 0.34 mmol) in 15 mL of 1,2-dichloroethane. Sodium triacetoxyborohydride (0.216 g, 1.02 mmol) was added and the mixture was stirred at room temperature under nitrogen for ca. 18 hours. Ethyl acetate was added and the reaction mixture was washed with saturated aqueous sodium bicarbonate. The organic phase was dried over sodium sulfate and the solvent was evaporated. Chromatography on silica gel with dichloromethane/methanol gave 0.111 g (52% yield) of desired product as a white solid.

### Step 4

### 1-Dimethylethyl (2S)-({[4-chloro-2-({[(2,4,6-trimethylphenyl)amino]carbonyl}amino)phenyl]carbonyl}amino)(cyclohexyl)ethanoate

Trifluoroacetic acid (0.5 mL, 6.5 mmol) was added to a solution of 1,1-dimethylethyl (2S)-cyclohexyl({[4′-[(dimethylamino)methyl]-3-({[(2,4,6-trimethylphenyl)amino]carbonyl}amino)-4-biphenylyl]carbonyl}amino)ethanoate (0.111 g, 0.18 mmol) in 5 mL of dichloromethane. The mixture was stirred at room temperature overnight. The solvent was evaporated and the residue was purified by chromatography on silica gel with dichloromethane/methanolic ammonia to give 0.057 g (46% yield) of desired product as the trifluoroacetic acid salt. ES MS m/z 571 (M+H).

### Example 214

### (2S)-Cyclohexyl{[(3-{[({2,6-dichloro-4-[(trifluoromethyl)oxy]phenyl}amino)carbonyl]amino}-4-biphenylyl)carbonyl]amino}ethanoic acid

### Step 1

### 1,1-Dimethylethyl (2S)-cyclohexyl{[(3-{[({2,6-dichloro-4-[(trifluoromethyl)oxy]phenyl}amino)carbonyl]amino}-4-biphenylyl)carbonyl]amino}ethanoate

1,3-Dichloro-2-isocyanato-5-[(trifluoromethyl)oxy]benzene (0.266 g, 0.98 mmol) was added to a solution of 1,1-dimethylethyl (2S)-{[(3-amino-4-biphenylyl)carbonyl]amino}(cyclohexyl)ethanoate (0.200 g, 0.49 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.251 g (75% yield) of desired product as a white solid.

### Step 2

### (2S)-Cyclohexyl{[(3-{[({2,6-dichloro-4-[(trifluoromethyl)oxy]phenyl}amino)carbonyl]amino}-4-biphenylyl)carbonyl]amino}ethanoic acid

Trifluoroacetic acid (0.5 mL, 6.5 mmol) was added to a solution of 1,1-dimethylethyl (2S)-cyclohexyl({[4′-[(dimethylamino)methyl]-3-({[(2,4,6-trimethylphenyl)amino]carbonyl}amino)-4-biphenylyl]carbonyl}amino)ethanoate (0.238 g, 0.35 mmol) in 5 mL of dichloromethane. The mixture was stirred at room temperature for 3 hours. The solvent was evaporated and the residue was triturated with methanol to give 0.120 g (55% yield) of desired product as a white solid. ES MS m/z 622 (M−H).

### Example 215

### (2S)-Cyclohexyl({[3-{[({2,6-dichloro-4-[(trifluoromethyl)oxy]phenyl}amino)carbonyl]amino}-4′-(methyloxy)-4-biphenylyl]carbonyl}amino)ethanoic acid

### Step 1

### Methyl 4′-(methyloxy)-3-nitro-4-biphenylcarboxylate

In each of two microwave reaction vials, a mixture of methyl 4-chloro-2-nitrobenzoate (0.500 g, 2.62 mmol), 4-methoxyphenyl]boronic acid (0.385 g, 2.55 mmol), trans-dichlorobis(tricyclohexylphosphine)palladium(II) (0.084 g, 0.115 mmol), cesium fluoride (1.95 g, 6.95 mmol), 2.5 mL of water and 7.5 mL of acetonitrile was heated in a microwave reactor at 150° C. for 5 minutes. The cooled reaction mixtures were combined, filtered through Celite, diluted with ethyl acetate, washed with water and dried over sodium sulfate. Chromatography on silica gel with hexane/ethyl acetate gave 1.02 g (76% yield) of an off-white solid.

### Step 2

### 4′-(Methyloxy)-3-nitro-4-biphenylcarboxylic acid

Lithium hydroxide (0.248 g, 10.33 mmol) was added to a solution of methyl 4′-(methyloxy)-3-nitro-4-biphenylcarboxylate (0.988 g, 3.44 mmol) in 30 mL of THF:methanol:water/4:1:1. The mixture was stirred at room temperature overnight and 1N aqueous HCl was added. The solvents were evaporated and the residue was extracted between ethyl acetate and water. The organic phase was dried over sodium sulfate and the solvent was evaporated to give 0.910 g (97% yield) of desired product as a yellow solid.

### Step 3

### 1,1-Dimethylethyl (2S)-cyclohexyl({[4′-(methyloxy)-3-nitro-4-biphenylyl]carbonyl}amino)ethanoate

HATU (0.570 g, 1.50 mmol) was added to a solution of 4′-(methyloxy)-3-nitro-4-biphenylcarboxylic acid (0.300 g, 1.10 mmol), 1,1-dimethylethyl (2S)-amino(cyclohexyl)ethanoate hydrochloride (0.274 g, 1.10 mmol) and diisopropylethylamine (0.29 mL, 1.65 mmol) in 15 mL of DMF. The mixture was stirred at room temperature overnight. The reaction mixture was extracted between ethyl acetate and water. The organic phase was washed with water and brine and dried over anhydrous sodium sulfate and the solvent was removed under vacuum. Chromatography on silica gel with hexane/ethyl acetate gave 0.467 g of desired product as a yellow solid.

### Step 4

### 1,1-Dimethylethyl (2S)-({[3-amino-4′-(methyloxy)-4-biphenylyl]carbonyl}amino)(cyclohexyl)ethanoate

A mixture of 1,1-dimethylethyl (2S)-cyclohexyl({[4′-(methyloxy)-3-nitro-4-biphenylyl]carbonyl}amino)ethanoate (0.461 g, 0.98 mmol) and 5% palladium on carbon (0.105 g, 0.049 mmol) in 25 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.422 g (98% yield) of desired product as a beige solid.

### Step 5

### 1,1-Dimethylethyl (2S)-cyclohexyl({[3-{[({2,6-dichloro-4-[(trifluoromethyl)oxy]phenyl}amino)carbonyl]amino}-4′-(methyloxy)-4-biphenylyl]carbonyl}amino)ethanoate

1,3-Dichloro-2-isocyanato-5-[(trifluoromethyl)oxy]benzene (0.266 g, 0.98 mmol) was added to a solution of 1,1-dimethylethyl (2S)-({[3-amino-4′-(methyloxy)-4-biphenylyl]carbonyl}amino)(cyclohexyl)ethanoate (0.220 g, 0.502 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.180 g (50% yield) of desired product as a white solid.

### Step 6

### (2S)-Cyclohexyl({[3-{[({2,6-dichloro-4-[(trifluoromethyl)oxy]phenyl}amino)carbonyl]amino}-4′-(methyloxy)-4-biphenylyl]carbonyl}amino)ethanoic acid

Trifluoroacetic acid (0.5 mL, 6.5 mmol) was added to a solution of. 1,1-dimethylethyl (2S)-cyclohexyl({[3-{[({2,6-dichloro-4-[(trifluoromethyl)oxy]phenyl}amino)carbonyl]amino}-4′-(methyloxy)-4-biphenylyl]carbonyl}amino)ethanoate (0.172 g, 0.24 mmol) in dichloromethane. The mixture was stirred at room temperature for overnight. The solvent was evaporated and the residue was triturated with methanol to give 0.040 g (25% yield) of desired product as a white solid. ES MS m/z 652 (M−H).

### Example 216

### (2S)-Cyclohexyl({[4′-(1-pyrrolidinylmethyl)-3-({[(2,4,6-trimethylphenyl)amino]carbonyl}amino)-4-biphenylyl]carbonyl}amino)ethanoic acid

### Step 1

### 1,1-Dimethylethyl (2S)-cyclohexyl ({[4′-(hydroxymethyl)-3-({[(2,4,6-trimethylphenyl)amino]carbonyl}amino)-4-biphenylyl]carbonyl}amino)ethanoate

A mixture of 1,1-dimethylethyl (2S)-({[4-chloro-2-({[(2,4,6-trimethylphenyl)amino]carbonyl}amino)phenyl]carbonyl}amino)(cyclohexyl)ethanoate (0.652 g, 1.24 mmol), [4-(hydroxymethyl)phenyl]boronic acid (0.207 g, 1.36 mmol), trans-dichlorobis(tricyclohexylphosphine)palladium(II) (0.046 g, 0.062 mmol), cesium fluoride (0.565 g, 3.72 mmol), 3 mL of water and 8 mL of acetonitrile was heated in a microwave reactor at 150° C. for 5 minutes. The cooled reaction mixture was filtered through Celite, diluted with ethyl acetate, washed with water and dried over sodium sulfate. Chromatography on silica gel with hexane/ethyl acetate gave 0.341 g (46% yield) of desired product as a white solid.

### Step 2

### 1,1-Dimethylethyl (2S)-cyclohexyl({[4′-formyl-3-({[(2,4,6-trimethylphenyl)amino]carbonyl}amino)-4-biphenylyl]carbonyl}amino)ethanoate

Manganese dioxide (0.98 g, 11.3 mmol) was added to a solution of 1,1-dimethylethyl (2S)-cyclohexyl({[4′-(hydroxymethyl)-3-({[(2,4,6-trimethylphenyl)amino]carbonyl}amino)-4-biphenylyl]carbonyl}amino)ethanoate (0.338 g, 0.56 mmol) in dichloromethane. The mixture was stirred at room temperature for 18 hours, filtered through Celite and the solvent was evaporated. Chromatography on silica gel gave 0.247 g (74% yield) of desired product as a white solid.

### Step 3

### 1,1-Dimethylethyl (2S)-cyclohexyl({[4′-(1-pyrrolidinylmethyl)-3-({[(2,4,6-trimethylphenyl)amino]carbonyl}amino)-4-biphenylyl]carbonyl}amino)ethanoate

Sodium triacetoxyborohydride (0.140 g, 0.66 mmol) was added to a solution of 1,1-dimethylethyl (2S)-cyclohexyl({[4′-formyl-3-({[(2,4,6-trimethylphenyl)amino]carbonyl}amino)-4-biphenylyl]carbonyl}amino)ethanoate (0.132 g, 0.22 mmol) and pyrrolidine (0.078 g, 1.10 mmol) in 5 mL of 1,2-dichloroethane. The mixture was stirred at room temperature for 1.5 hours. The reaction mixture was diluted with ethyl acetate and washed with saturated aqueous sodium bicarbonate. The organic phase was dried over sodium sulfate and the solvent was removed under vacuum. The residue was purified by chromatography on silica gel with dichloromethane/methanol to give 0.091 g (63% yield) of desired product as a colorless resin.

### Step 4

### (2S)-Cyclohexyl({[4′-(l-pyrrolidinylmethyl)-3-({[(2,4,6-trimethylphenyl)amino]carbonyl}amino)-4-biphenylyl]carbonyl}amino)ethanoic acid

Trifluoroacetic acid (0.5 mL, 6.5 mmol) was added to a solution of 1,1-dimethylethyl (2S)-cyclohexyl({[4′-(1-pyrrolidinylmethyl)-3-({[(2,4,6-trimethylphenyl)amino]carbonyl}amino)-4-biphenylyl]carbonyl}amino)ethanoate (0.091 g, 0.14 mmol) in dichloromethane. The mixture was stirred at room temperature overnight. The solvent was evaporated and the residue was purified by chromatography on silica gel with dichloromethane/methanol to give 0.026 g (31% yield) of desired product as a white solid. ES MS m/z 595 (M−H).

### Example 217

### (2S)-cyclohexyl({[4′-(4-morpholinylmethyl)-3-({[(2,4,6-trimethylphenyl)amino]carbonyl}amino)-4-biphenylyl]carbonyl}amino)ethanoic acid

### Step 1

### 1,1-Dimethylethyl (2S)-cyclohexyl({[4′-(tetrahydro-2H-pyran-4-ylmethyl)-3-({[(2,4,6-trimethylphenyl)amino]carbonyl}amino)-4-biphenylyl]carbonyl}amino)ethanoate

Sodium triacetoxyborohydride (0.114 g, 0.54 mmol) was added to a solution of 1,1-dimethylethyl (2S)-cyclohexyl({[4′-formyl-3-({[(2,4,6-trimethylphenyl)amino]carbonyl}amino)-4-biphenylyl]carbonyl}amino)ethanoate (0.108 g, 0.18 mmol) and morpholine (0.079 g, 0.90 mmol) in 5 mL of 1,2-dichloroethane. The mixture was stirred at room temperature for 1.5 hours. The reaction mixture was diluted with ethyl acetate and washed with saturated aqueous sodium bicarbonate. The organic phase was dried over sodium sulfate and the solvent was removed under vacuum. The residue was purified by chromatography on silica gel with dichloromethane/methanol to give 0.127 g desired product as a colorless gum.

### Step 2

### (2S)-cyclohexyl({[4′-(4-morpholinylmethyl)-3-({[(2,4,6-trimethylphenyl)amino]carbonyl}amino)-4-biphenylyl]carbonyl}amino)ethanoic acid

Trifluoroacetic acid (0.5 mL, 6.5 mmol) was added to a solution of 1,1-dimethylethyl (2S)-cyclohexyl({[4′-(tetrahydro-2H-pyran-4-ylmethyl)-3-({[(2,4,6-trimethylphenyl)amino]carbonyl}amino)-4-biphenylyl]carbonyl}amino)ethanoate (0.120 g, 0.18 mmol) in 4 mL of dichloromethane. The mixture was stirred at room temperature overnight. The solvent was evaporated and the residue was purified by chromatography on silica gel with dichloromethane/methanol to give 0.046 g (35% yield) of desired product as a white solid. ES MS m/z 611 (M−H).

### Example 218

### (2S)-Cyclohexyl({[4′-(ethyloxy)-3-({[(2,4,6-trimethylphenyl)amino]carbonyl}amino)-4-biphenylyl]carbonyl}amino)ethanoic acid

### Step 1

### Methyl(2S)-cyclohexyl({[4′-(ethyloxy)-3-({[(2,4,6-trimethylphenyl)amino]carbonyl}amino)-4-biphenylyl]carbonyl}amino)ethanoate

A mixture of methyl(2S)-({[4-chloro-2-({[(2,4,6-trimethylphenyl)amino]carbonyl}amino)phenyl]carbonyl}amino)(cyclohexyl)ethanoate (0.200 g, 0.41 mmol), [4-(ethyloxy)phenyl]boronic acid (0.075 g, 0.45 mmol), trans-dichlorobis(tricyclohexylphosphine)palladium(II) (0.015 g, 0.021 mmol), cesium fluoride (0.187 g, 1.23 mmol), 1.5 mL of water and 4 mL of acetonitrile was heated in a microwave reactor at 150° C. for 5 minutes. The cooled reaction mixture was diluted with ethyl acetate, washed with water and dried over sodium sulfate. Chromatography on silica gel with hexane/ethyl acetate gave 0.078 g (33% yield) of a white solid containing 85-90% desired product.

### Step 2

### (2S)-Cyclohexyl({[4′-(ethyloxy)-3-({[(2,4,6-trimethylphenyl)amino]carbonyl}amino)-4-biphenylyl]carbonyl}amino)ethanoic acid

Lithium hydroxide (0.033 g, 1.37 mmol) was added to a solution of methyl(2S)-cyclohexyl ({[4′-(ethyloxy)-3-({[(2,4,6-trimethylphenyl)amino]carbonyl}amino)-4-biphenylyl]carbonyl}amino)ethanoate (0.078 g, 0.14 mmol) in 3 mL of THF:methanol:water/4:1:1. The mixture was stirred at room temperature overnight and 1N aqueous HCl was added. The solvent was evaporated and the residue was extracted between ethyl acetate and water. The organic phase was dried over sodium sulfate and the solvent was evaporated. The residue was purified by chromatography on silica gel with hexane/ethyl acetate to give 0.041 g (52% yield) of desired product as a white solid. ES MS m/z 556 (M−H).

### Example 219

### N-{[4′-(methyloxy)-3-({[(2,4,6-trimethylphenyl)amino]carbonyl}amino)-4-biphenylyl]carbonyl}-L-norleucine

### Step 1

### Methyl 4′-(methyloxy)-3-nitro-4-biphenylcarboxylate

Methyl 4-chloro-2-nitrobenzoate (1.79 g, 8.31 mmol), 4-methoxyphenylboronic acid (1.39 g, 9.14 mmol), trans-dichlorobis(tricyclohexylphosphine)palladium(II) (0.306 g, 0.41 mmol) and cesium fluoride (3.79 g, 24.9 mmol) were mixed in 40 mL of acetonitrile:water/3:1 was heated in a microwave reactor at 150° C. for 5 minutes. The cooled reaction mixture was filtered through Celite, diluted with ethyl acetate and washed with water and brine. The organic phase was dried over anhydrous sodium sulfate and the solvent was evaporated. Chromatography on silica gel with hexane/ethyl acetate gave 0.604 g (25% yield) of desired product.

### Step 2

### 4′-(Methyloxy)-3-nitro-4-biphenylcarboxylic acid

Lithium hydroxide (0.135 g, 5.64 mmol) was added to a solution of methyl 4′-(methyloxy)-3-nitro-4-biphenylcarboxylate (0.540 g, 1.88 mmol) in THF:methanol:water/5:1:1. The mixture was stirred at room temperature overnight. The solvent was evaporated and 1N aqueous HCl was added to the residue. The resulting suspension was extracted between ethyl acetate and water. The organic phase was dried over anhydrous sodium sulfate and the solvent was removed under vacuum to give 0.328 g (64% yield) of desired product.

### Step 3

### Methyl N-{[4′-(methyloxy)-3-nitro-4-biphenylyl]carbonyl}-L-norleucinate

HATU (0.334 g, 0.88 mmol) was added to a solution of 4′-(methyloxy)-3-nitro-4-biphenylcarboxylic acid (0.162 g, 0.59 mmol), methyl L-norleucinate hydrochloride (0.118 g, 0.65 mmol), and diisopropylamine (0.15 mL, 0.88 mmol) in DMF. The mixture was stirred at room temperature overnight, and then diluted with ethyl acetate, and washed with water and brine. The organic phase was dried over anhydrous sodium sulfate and the solvent was removed under vacuum to give 0.203 g (86% yield) of desired product as an off-white solid.

### Step 4

### Methyl N-{[3-amino-4′-(methyloxy)-4-biphenylyl]carbonyl}-L-norleucinate

A mixture of methyl N-{[4′-(methyloxy)-3-nitro-4-biphenylyl]carbonyl}-L-norleucinate (0.203 g, 0.51 mmol) and 5% palladium on carbon (0.054 g, 0.025 mmol) in ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.159 g (84% yield) of desired product.

### Step 5

### Methyl N-{[4′-(methyloxy)-3-({[(2,4,6-trimethylphenyl)amino]carbonyl}amino)-4-biphenylyl]carbonyl}-L-norleucinate

2,4,6-Trimethylphenylisocyanate (0.198 g, 1.23 mmol) was added to a solution of methyl N-{[3-amino-4′-(methyloxy)-4-biphenylyl]carbonyl}-L-norleucinate (0.152 g, 0.41 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.173 g (79% yield) of desired product as a white solid.

### Step 6

### N-{[4′-(methyloxy)-3-({[(2,4,6-trimethylphenyl)amino]carbonyl}amino)-4-biphenylyl]carbonyl}-L-norleucine

Lithium hydroxide (0.074 g, 3.1 mmol) was added to a solution of methyl N-{[4′-(methyloxy)-3-({[(2,4,6-trimethylphenyl)amino]carbonyl}amino)-4-biphenylyl]carbonyl}-L-norleucinate (0.164 g, 0.31 mmol) in THF:methanol:water/4:1:1. The mixture was stirred at room temperature overnight.

The solvent was evaporated and 1N aqueous HCl was added to the residue. The resulting suspension was extracted between ethyl acetate and water. The organic phase was dried over anhydrous sodium sulfate and the solvent was removed under vacuum to give 0.124 g (77% yield) of desired product. ES MS m/z 516 (M−H).

### Example 220

### (2S)-Cyclohexyl({[4-(methylsulfonyl)-2-({[(2,4,6-trimethylphenyl)amino]carbonyl}amino)phenyl]carbonyl}amino)ethanoic acid

### Step 1

### 1,1-Dimethylethyl (2S)-cyclohexyl({[4-(methylsulfonyl)-2-nitrophenyl]carbonyl}amino)ethanoate

HATU (1.16 g, 3.06 mmol) was added to a solution of 4-(methylsulfonyl)-2-nitrobenzoic acid (0.500 g, 2.04 mmol), 1,1-dimethylethyl (2S)-amino(cyclohexyl)ethanoate hydrochloride (0.509 g, 2.04 mmol), and diisopropylamine (0.53 mL, 3.06 mmol) in DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.668 g (74% yield) of desired product as a white solid.

### Step 2

### 1,1-Dimethylethyl (2S)-({[2-amino-4-(methylsulfonyl)phenyl]carbonyl}amino)(cyclohexyl)ethanoate

A mixture of 1,1-dimethylethyl (2S)-cyclohexyl({[4-(methylsulfonyl)-2-nitrophenyl]carbonyl}amino)ethanoate (0.641 g, 1.46 mmol) and 5% palladium on carbon (0.155 g, 0.073 mmol) in 120 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.557 g (93% yield) of desired product as a gray solid.

### Step 3

### 1,1-Dimethylethyl (2S)-cyclohexyl({[4-(methylsulfonyl)-2-({[(2,4,6-trimethylphenyl)amino]carbonyl}amino)phenyl]carbonyl}amino)ethanoate

2,4,6-Trimethylphenylisocyanate (0.353 g, 2.19 mmol) was added to a solution of 1,1-Dimethylethyl (2S)-({[2-amino-4-(methylsulfonyl)phenyl]carbonyl}amino)(cyclohexyl)ethanoate (0.300 g, 0.73 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.274 g (66% yield) of desired product as a white solid.

### Step 4

### 2S)-Cyclohexyl({[4-(methylsulfonyl)-2-({[(2,4,6-trimethylphenyl)amino]carbonyl}amino)phenyl]carbonyl}amino)ethanoic acid

Trifluoroacetic acid (0.50 mL, 6.49 mmol) was added to a solution of, 1-dimethylethyl (2S)-cyclohexyl({[4-(methylsulfonyl)-2-({[(2,4,6-trimethylphenyl)amino]carbonyl}amino)phenyl]carbonyl}amino)ethanoate (0.270 g, 0.47 mmol) in 10 mL of dichloromethane. The mixture was stirred at room temperature overnight and the solvent was evaporated. The residue was purified by chromatography on silica gel with hexane/ethyl acetate to give 0.115 g (48% yield) of desired product as a white solid. ES MS m/z 514 (M−H).

### Example 221

### 1-({[4-Fluoro-2-({[(2,4,6-trimethylphenyl)amino]carbonyl}amino)phenyl]carbonyl}amino)cycloheptane-carboxylic acid

### Step 1

### Methyl 1-{[(4-fluoro-2-nitrophenyl)carbonyl]amino}cycloheptanecarboxylate

HATU (1.54 g, 4.05 mmol) was added to a solution of 4-fluoro-2-nitrobenzoic acid (0.500 g, 2.70 mmol), methyl 1-aminocycloheptanecarboxylate hydrochloride (0.560 g, 2.70 mmol), and diisopropyethylamine (0.70 mL, 4.05 mmol) in 20 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.475 g (52% yield) of desired product as a white solid.

### Step 2

### Methyl 1-{[(2-amino-4-fluorophenyl)carbonyl]amino}cycloheptanecarboxylate

A mixture of methyl 1-{[(4-fluoro-2-nitrophenyl)carbonyl]amino}cycloheptanecarboxylate (0.468 g, 1.38 mmol) and 5% palladium on carbon (0.147 g, 0.069 mmol) in 30 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.400 g (94% yield) of desired product as an off-white solid.

### Step 3

### Methyl 1-({[4-fluoro-2-({[(2,4,6-trimethylphenyl)amino]carbonyl}amino)phenyl]carbonyl}amino)cycloheptane-carboxylate

2,4,6-Trimethylphenylisocyanate (0.624 g, 3.88 mmol) was added to a solution of methyl 1-{[(2-amino-4-fluorophenyl)carbonyl]amino}cycloheptanecarboxylate (0.398 g, 1.29 mmol) in 10 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.439 g (72% yield) of desired product as a white solid.

### Step 4

### 1-({[4-Fluoro-2-({[(2,4,6-trimethylphenyl)amino]carbonyl}amino)phenyl]carbonyl}amino)cycloheptanecarboxylic acid

Lithium hydroxide (0.226 g, 9.4 mmol) was added to a solution of methyl 1-({[4-fluoro-2-({[(2,4,6-trimethylphenyl)amino]carbonyl}amino)phenyl]carbonyl}amino)cycloheptanecarboxylate (0.439 g, 0.94 mmol) in THF:methanol:water/2.5:1:1. The mixture was heated to 50° C. for one hour. The solvent was evaporated, and 1N aqueous HCl was added to the residue. The resulting suspension was extracted with ethyl acetate and the organic layer was dried over sodium sulfate. The solvent was removed under vacuum to give 0.401 g (94% yield) of the desired product as a white solid. ES MS m/z 454 (M−H).

### Example 222

### 1-({[4′-(methyloxy)-3-({[(2,4,6-trimethylphenyl)amino]carbonyl}amino)-4-biphenylyl]carbonyl}amino)cycloheptanecarboxylic acid

### Step 1

### Methyl 4′-(methyloxy)-3-nitro-4-biphenylcarboxylate

In each of two microwave reaction vials, a mixture of methyl 4-chloro-2-nitrobenzoate (0.700 g, 3.25 mmol), 4-methoxyphenylboronic acid (0.543 g, 3.57 mmol), trans-dichlorobis(tricyclohexylphosphine)palladium(II) (0.120 g, 0.16 mmol), cesium fluoride (1.48 g, 9.75 mmol), 3 mL of water and 8 mL of acetonitrile was heated in a microwave reactor at 150° C. for 5 minutes. The cooled reaction mixtures were combined, diluted with ethyl acetate, washed with water and dried over sodium sulfate. Chromatography on silica gel with hexane/ethyl acetate gave 1.44 g (77% yield) of desired product as an off-white solid.

### Step 2

### 4′-(Methyloxy)-3-nitro-4-biphenylcarboxylic acid

Lithium hydroxide (0.36 g, 14.8 mmol) was added to a solution of methyl 4′-(methyloxy)-3-nitro-4-biphenylcarboxylate (1.42 g, 4.94 mmol) in 25 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 1.26 g (93% yield) of desired product as a yellow solid.

### Step 3

### Methyl 1-({[4′-(methyloxy)-3-nitro-4-biphenylyl]carbonyl}amino)cycloheptanecarboxylate

HATU (1.04 g, 2.74 mmol) was added to a solution of 4′-(methyloxy)-3-nitro-4-biphenylcarboxylic acid (0.500 g, 1.83 mmol), methyl 1-aminocycloheptanecarboxylate hydrochloride (0.380 g, 1.83 mmol), and diisopropylethylamine (0.48 mL, 2.74 mmol) in 20 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.588 g (75% yield) of desired product as a yellow solid.

### Step 4

### Methyl 1-({[3-amino-4′-(methyloxy)-4-biphenylyl]carbonyl}amino)cycloheptanecarboxylate

A mixture of methyl 1-({[4′-(methyloxy)-3-nitro-4-biphenylyl]carbonyl}amino)cycloheptanecarboxylate (0.584 g, 1.37 mmol) and 5% palladium on carbon (0.146 g, 0.069 mmol) in 35 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.516 g (95% yield) of desired product as an off-white solid.

### Step 5

### Methyl 1-({[4′-(methyloxy)-3-({[(2,4,6-trimethylphenyl)amino]carbonyl}amino)-4-biphenylyl]carbonyl}amino)cycloheptanecarboxylate

2,4,6-Trimethylphenylisocyanate (0.244 g, 1.51 mmol) was added to a solution of methyl 1-({[3-amino-4′-(methyloxy)-4-biphenylyl]carbonyl}amino)cycloheptanecarboxylate (0.200 g, 0.505 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.179 g (64% yield) of desired product as a white solid.

### Step 6

### 1-({[4′-(methyloxy)-3-({[(2,4,6-trimethylphenyl)amino]carbonyl}amino)-4-biphenylyl]carbonyl}amino)cycloheptanecarboxylic acid

Lithium hydroxide (0.076 g, 3.2 mmol) was added to a solution of methyl 1-({[4′-(methyloxy)-3-({[(2,4,6-trimethylphenyl)amino]carbonyl}amino)-4-biphenylyl]carbonyl}amino)cycloheptanecarboxylate (0.176 g, 0.32 mmol) in 3 mL of THF:methanol:water/4:1:1. The mixture was heated at 50° C. overnight. The solvent was evaporated and 1N aqueous hydrochloric acid was added to the residue. The resulting suspension was extracted with ethyl acetate, dried over anhydrous sodium sulfate and the solvent was removed under vacuum to give 0.155 g (89% yield) of desired product as a yellow solid. ES MS m/z 542 (M−H).

### Example 223

### (2S)-Cyclohexyl({[4′-fluoro-3-({[(2,4,6-trimethylphenyl)amino]carbonyl}amino)-4-biphenylyl]carbonyl}amino)ethanoic acid

### Step 1

### Methyl(2S)-cyclohexyl({[4′-fluoro-3-({[(2,4,6-trimethylphenyl)amino]carbonyl}amino)-4-biphenylyl]carbonyl}amino)ethanoate

A mixture of methyl(2S)-({[4-chloro-2-({[(2,4,6-trimethylphenyl)amino]carbonyl}amino)phenyl]carbonyl}amino)(cyclohexyl)ethanoate (0.200 g, 0.41 mmol), 4-fluorophenylboronic acid (0.063 g, 0.45 mmol), trans-dichlorobis(tricyclohexylphosphine)palladium(II) (0.015 g, 0.0205 mmol), cesium fluoride (0.187 g, 1.23 mmol), 1 mL of water and 3 mL of acetonitrile was heated in a microwave reactor at 150° C. for 5 minutes. The cooled reaction mixture was filtered through Celite, diluted with ethyl acetate, washed with water, and dried over sodium sulfate. Chromatography on silica gel with hexane/ethyl acetate gave 0.165 g of a white solid containing about 85% desired product.

### Step 2

### (2S)-Cyclohexyl({[4′-fluoro-3-({[(2,4,6-trimethylphenyl)amino]carbonyl}amino)-4-biphenylyl]carbonyl}amino)ethanoic acid

Lithium hydroxide (0.071 g, 2.95 mmol) was added to a solution of methyl(2S)-cyclohexyl({[4′-fluoro-3-({[(2,4,6-trimethylphenyl)amino]carbonyl}amino)-4-biphenylyl]carbonyl}amino)ethanoate (0.161 g, 0.29 mmol) in 3 mL of THF:methanol:water/4:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum. Chromatography on silica gel with hexane/ethyl acetate gave 0.075 g (49% yield) of desired product as a white solid. ES MS m/z 530 (M−H).

### Example 224

### (2S)-({[4-(1,3-Benzodioxol-5-yl)-2-({[(2,4,6-trimethylphenyl)amino]carbonyl}amino)phenyl]carbonyl}amino)(cyclohexyl)-ethanoic acid

### Step 1

### Methyl(2S)-({[4-(1,3-benzodioxol-5-yl)-2-({[(2,4,6-trimethylphenyl)amino]carbonyl}amino)phenyl]carbonyl}amino)(cyclohexyl)ethanoate

A mixture methyl(2S)-({[4-chloro-2-({[(2,4,6-trimethylphenyl)amino]carbonyl}amino)phenyl]carbonyl}amino)(cyclohexyl)ethanoate (0.200 g, 0.41 mmol), 1,3-benzodioxol-5-ylboronic acid (0.075 g, 0.45 mmol), trans-dichlorobis(tricyclohexylphosphine)palladium(II) (0.015 g, 0.0205 mmol), cesium fluoride (0.187 g, 1.23 mmol), 1 mL of water and 3 mL of acetonitrile was heated in a microwave reactor at 150° C. for 5 minutes. The cooled reaction mixture was filtered through Celite, diluted with ethyl acetate, washed with water, and dried over sodium sulfate. Chromatography on silica gel with hexane/ethyl acetate gave 0.182 g of a white solid containing about 85% desired product.

### Step 2

### (2S)-({[4-(1,3-Benzodioxol-5-yl)-2-({[(2,4,6-trimethylphenyl)amino]carbonyl}amino)phenyl]carbonyl}amino)(cyclohexyl)-ethanoic acid

Lithium hydroxide (0.069 g, 2.88 mmol) was added to a solution of methyl(2S)-({[4-(1,3-benzodioxol-5-yl)-2-({[(2,4,6-trimethylphenyl)amino]carbonyl}amino)phenyl]carbonyl}amino)(cyclohexyl)ethanoate (0.165 g, 0.29 mmol) in 3 mL of THF:methanol:water/4:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum. Chromatography on silica gel with hexane/ethyl acetate gave 0.103 g (64% yield) of desired product as a white solid. ES MS m/z 556 (M−H).

### Example 225

### O-(1,1-Dimethylethyl)-N-{[4′-(methyloxy)-3-({[(2,4,6-trimethylphenyl)amino]carbonyl}amino)-4-biphenylyl]carbonyl}-L-threonine

### Step 1

### Methyl O-(1,1-dimethylethyl)-N-{[4′-(methyloxy)-3-nitro-4-biphenylyl]carbonyl}-L-threoninate

HATU (0.627 g, 1.65 mmol) was added to a solution of 4′-(Methyloxy)-3-nitro-4-biphenylcarboxylic acid (0.300 g, 1.10 mmol), methyl O-(1,1-dimethylethyl)-L-threoninate hydrochloride (0.248 g, 1.10 mmol), and diisopropylethylamine (0.29 mL, 1.65 mmol) in 15 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.329 g (67% yield) of desired product as a light yellow solid.

### Step 2

### Methyl N-{[3-amino-4′-(methyloxy)-4-biphenylyl]carbonyl}-O-(1,1-dimethylethyl)-L-threoninate

A mixture of methyl O-(1,1-dimethylethyl)-N-{[4′-(methyloxy)-3-nitro-4-biphenylyl]carbonyl}-L-threoninate (0.324 g, 0.73 mmol) and 5% palladium on carbon (0.078 g, 0.036 mmol) in 20 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.297 g (98% yield) of desired product as an off-white solid.

### Step 3

### Methyl O-(1,1-dimethylethyl)-N-{[4′-(methyloxy)-3-({[(2,4,6-trimethylphenyl)amino]carbonyl}amino)-4-biphenylyl]carbonyl}-L-threoninate

2,4,6-Trimethylphenylisocyanate (0.334 g, 2.07 mmol) was added to a solution of methyl N-{[3-amino-4′-(methyloxy)-4-biphenylyl]carbonyl}-O-(1,1-dimethylethyl)-L-threoninate (0.286 g, 0.69 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.327 g (82% yield) of desired product as a white solid.

### Step 4

### O-(1,1-Dimethylethyl)-N-{[4′-(methyloxy)-3-({[(2,4,6-trimethylphenyl)amino]carbonyl}amino)-4-biphenylyl]carbonyl}-L-threonine

Lithium hydroxide (0.133 g, 5.5 mmol) was added to a solution of methyl O-(1,1-dimethylethyl)-N-{[4′-(methyloxy)-3-({[(2,4,6-trimethylphenyl)amino]carbonyl}amino)-4-biphenylyl]carbonyl}-L-threoninate (0.319 g, 0.55 mmol) in 6 mL of THF:methanol:water/4:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.266 g (86% yield) of desired product as a white solid. ES MS m/z 560 (M−H).

### Example 226

### O-(1,1-Dimethylethyl)-N-{[4-fluoro-2-({[(2,4,6-trimethylphenyl)amino]carbonyl}amino)phenyl]carbonyl}-L-threonine

### Step 1

### Methyl O-(1 μl-dimethylethyl)-N-[(4-fluoro-2-nitrophenyl)carbonyl]-L-threoninate

HATU (1.54 g, 4.05 mmol) was added to a solution of 4-fluoro-2-nitrobenzoic acid (0.500 g, 2.70 mmol), methyl O-(1,1-dimethylethyl)-L-threoninate hydrochloride (0.609 g, 2.70 mmol), and diisopropylethylamine (0.70 mL, 4.05 mmol) in 20 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.621 g (65% yield) of desired product as a colorless gum.

### Step 2

### Methyl N-[(2-amino-4-fluorophenyl)carbonyl]-O-(1,1-dimethylethyl)-L-threoninate

A mixture of methyl O-(1,1-dimethylethyl)-N-[(4-fluoro-2-nitrophenyl)carbonyl]-L-threoninate (0.586 g, 1.65 mmol) and 5% palladium on carbon (0.175 g, 0.0825 mmol) in 35 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.534 g (99% yield) of desired product as a colorless gum.

### Step 3

### Methyl O-(1,1-dimethylethyl)-N-{[4-fluoro-2-({[(2,4,6-trimethylphenyl)amino]carbonyl}amino)phenyl]carbonyl}-L-threoninate

2,4,6-Trimethylphenylisocyanate (0.345 g, 2.14 mmol) was added to a solution of methyl N-[(2-amino-4-fluorophenyl)carbonyl]-O-(1,1-dimethylethyl)-L-threoninate (0.233 g, 0.71 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.292 g (84% yield) of desired product as a white solid.

### Step 4

### O-(1,1-Dimethylethyl)-N-{[4-fluoro-2-({[(2,4,6-trimethylphenyl)amino]carbonyl}amino)phenyl]carbonyl}-L-threonine

Lithium hydroxide (0.140 g, 5.85 mmol) was added to a solution of methyl O-(1,1-dimethylethyl)-N-{[4-fluoro-2-({[(2,4,6-trimethylphenyl)amino]carbonyl}amino)phenyl]carbonyl}-L-threoninate (0.285 g, 0.585 mmol) in 6 mL of THF:methanol:water/4:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum. Chromatography on silica gel with hexane/ethyl acetate gave 0.110 g (40% yield) of desired product as a white solid. APCI MS m/z 472 (M−H).

### Example 227

### 1-({[3′,4′-Difluoro-3-({[(2,4,6-trimethylphenyl)amino]carbonyl}amino)-4-biphenylyl]carbonyl}amino)cyclooctanecarboxylic acid

### Step 1

### Methyl 1-{[(3′,4′-difluoro-3-nitro-4-biphenylyl)carbonyl]amino}cyclooctanecarboxylate

HATU (0.467 g, 1.23 mmol) was added to a solution of 3′,4′-difluoro-3-nitro-4-biphenylcarboxylic acid (0.230 g, 0.82 mmol), methyl 2-amino-2-ethyloctanoate (0.152 g, 0.82 mmol), and diisopropylethylamine (0.21 mL, 1.23 mmol) in 10 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.248 g (68% yield) of desired product as a white solid.

### Step 2

### Methyl 1-{[(3-amino-3′,4′-difluoro-4-biphenylyl)carbonyl]amino}cyclooctanecarboxylate

A mixture of methyl 1-{[(3′,4′-difluoro-3-nitro-4-biphenylyl)carbonyl]amino}cyclooctanecarboxylate (0.243 g, 0.54 mmol) and 5% palladium on carbon (0.058 g, 0.027 mmol) in 15 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.231 g of desired product as a white solid.

### Step 3

### Methyl 1-({[3′,4′-difluoro-3-({[(2,4,6-trimethylphenyl)amino]carbonyl}amino)-4-biphenylyl]carbonyl}amino)cyclooctanecarboxylate

2,4,6-Trimethylphenylisocyanate (0.258 g, 1.60 mmol) was added to a solution of methyl 1-{[(3-amino-3′,4′-difluoro-4-biphenylyl)carbonyl]amino}cyclooctanecarboxylate (0.222 g, 0.53 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.235 g (77% yield) of desired product as a white solid.

### Step 4

### 1-({[3′,4′-Difluoro-3-({[(2,4,6-trimethylphenyl)amino]carbonyl}amino)-4-biphenylyl]carbonyl}amino)cyclooctanecarboxylic acid

Lithium hydroxide (0.096 g, 4.0 mmol) was added to a solution of methyl 1-({[3′,4′-difluoro-3-({[(2,4,6-trimethylphenyl)amino]carbonyl}amino)-4-biphenylyl]carbonyl}amino)cyclooctanecarboxylate (0.231 g, 0.40 mmol) in 3 mL of THF:methanol:water/4:1:1. The mixture was heated at 50° C. overnight. The solvent was evaporated and 1N aqueous hydrochloric acid was added to the residue. The resulting suspension was extracted with ethyl acetate, dried over anhydrous sodium sulfate and the solvent removed under vacuum to give 0.220 g (98% yield) of desired product as a white solid. ES MS m/z 564 (M+H).

### Example 228

### (2S)-Cyclohexyl({[4-(2,3-dihydro-1,4-benzodioxin-6-yl)-2-({[(2,4,6-trimethylphenyl)amino]carbonyl}amino)phenyl]carbonyl}amino)ethanoic acid

### Step 1

### Methyl(2S)-cyclohexyl({[4-(2,3-dihydro-1,4-benzodioxin-6-yl)-2-({[(2,4,6-trimethylphenyl)amino]carbonyl}amino)phenyl]carbonyl}amino)ethanoate

A mixture of methyl(2S)-({[4-chloro-2-({[(2,4,6-trimethylphenyl)amino]carbonyl}amino)phenyl]carbonyl}amino)(cyclohexyl)ethanoate (0.200 g, 0.41 mmol), 2,3-dihydro-1,4-benzodioxin-6-ylboronic acid (0.0815 g, 0.45 mmol), trans-dichlorobis(tricyclohexylphosphine)palladium(II) (0.015 g, 0.0205 mmol), cesium fluoride (0.186 g, 1.23 mmol), 0.5 mL of water and 3 mL of acetonitrile was heated in a microwave reactor at 150° C. for 5 minutes. The cooled reaction mixture was filtered through Celite, diluted with ethyl acetate, washed with water, and dried over sodium sulfate. Chromatography on silica gel with hexane/ethyl acetate gave 0.187 g (78% yield) of desired product as a white solid.

### Step 2

### (2S)-Cyclohexyl({[4-(2,3-dihydro-1,4-benzodioxin-6-yl)-2-({[(2,4,6-trimethylphenyl)amino]carbonyl}amino)phenyl]carbonyl}amino)ethanoic acid

Lithium hydroxide (0.077 g, 3.2 mmol) was added to a solution of methyl(2S)-cyclohexyl({[4-(2,3-dihydro-1,4-benzodioxin-6-yl)-2-({[(2,4,6-trimethylphenyl)amino]carbonyl}amino)phenyl]carbonyl}amino)ethanoate (0.187 g, 0.32 mmol) in 3 mL of THF:methanol:water/4:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum. Chromatography on silica gel with hexane/ethyl acetate gave 0.040 g (22% yield) of desired product as a white solid. ES MS m/z 572 (M+H).

### Example 229

### (2S)-({[3′,4′-Bis(methyloxy)-3-({[(2,4,6-trimethylphenyl)amino]carbonyl}amino)-4-biphenylyl]carbonyl}amino)(cyclohexyl)ethanoic acid

### Step 1

### Methyl(2S)-({[3′,4′-bis(methyloxy)-3-({[(2,4,6-trimethylphenyl)amino]carbonyl}amino)-4-biphenylyl]carbonyl}amino)(cyclohexyl)ethanoate

A mixture of methyl(2S)-({[4-chloro-2-({[(2,4,6-trimethylphenyl)amino]carbonyl}amino)phenyl]carbonyl}amino)(cyclohexyl)ethanoate (0.200 g, 0.41 mmol), [3,4-bis(methyloxy)phenyl]boronic acid (0.082 g, 0.45 mmol), trans-dichlorobis(tricyclohexylphosphine)palladium(II) (0.015 g, 0.0205 mmol), cesium fluoride (0.186 g, 1.23 mmol), 0.5 mL of water and 3 mL of acetonitrile was heated in a microwave reactor at 150° C. for 5 minutes. The cooled reaction mixture was filtered through Celite, diluted with ethyl acetate, washed with water, and dried over sodium sulfate. Chromatography on silica gel with hexane/ethyl acetate gave 0.086 g (36% yield) of desired product as a white solid.

### Step 2

### (2S)-({[3′,4′-Bis(methyloxy)-3-({[(2,4,6-trimethylphenyl)amino]carbonyl}amino)-4-biphenylyl]carbonyl}amino)(cyclohexyl)ethanoic acid

Lithium hydroxide (0.035 g, 1.5 mmol) was added to a solution of methyl(2S)-({[3′,4′-bis(methyloxy)-3-({[(2,4,6-trimethylphenyl)amino]carbonyl}amino)-4-biphenylyl]carbonyl}amino)(cyclohexyl)ethanoate (0.086 g, 0.15 mmol) in 2.5 mL of THF:methanol:water/4:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum. Chromatography on silica gel with hexane/ethyl acetate gave 0.016 g (19% yield) of desired product as a white solid. ES MS m/z 574 (M+H).

### Example 230

### (2S)-Cyclohexyl({[4,5-difluoro-2-({[(2,4,6-trimethylphenyl)amino]carbonyl}amino)phenyl]carbonyl}amino)ethanoic acid

### Step 1

### Methyl(2S)-cyclohexyl{[(4,5-difluoro-2-nitrophenyl)carbonyl]amino}ethanoate

HATU (1.402 g, 3.69 mmol) was added to a solution of 4,5-difluoro-2-nitrobenzoic acid (0.500 g, 2.46 mmol), methyl(2S)-amino(cyclohexyl)ethanoate hydrochloride(0.510 g, 2.46 mmol), and diisopropylethylamine (0.64 mL, 3.69 mmol) in 20 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.853 g of crude desired product as a yellow oil. This material was carried on to the next step without further purification.

### Step 2

### Methyl(2S)-{[(2-amino-4,5-difluorophenyl)carbonyl]amino}(cyclohexyl)ethanoate

A mixture of methyl(2S)-cyclohexyl{[(4,5-difluoro-2-nitrophenyl)carbonyl]amino}ethanoate (0.850 g, 2.39 mmol) and 5% palladium on carbon (0.254 g, 0.119 mmol) in 30 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated. The residue was purified by chromatography on silica gel with hexane/ethyl acetate to give 0.333 g (43% yield) of desired product as a white solid.

### Step 3

### Methyl(2S)-cyclohexyl({[4,5-difluoro-2-({[(2,4,6-trimethylphenyl)amino]carbonyl}amino)phenyl]carbonyl}amino)ethanoate

2,4,6-Trimethylphenylisocyanate (0.148 g, 0.921 mmol) was added to a solution of methyl(2S)-{[(2-amino-4,5-difluorophenyl)carbonyl]amino}(cyclohexyl)ethanoate (0.100 g, 0.307 mmol) in 3 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.152 g of desired product as a white solid.

### Step 4

### (2S)-Cyclohexyl({[4,5-difluoro-2-({[(2,4,6-trimethylphenyl)amino]carbonyl}amino)phenyl]carbonyl}amino)ethanoic acid

Lithium hydroxide (0.057 g, 2.4 mmol) was added to a solution of methyl(2S)-cyclohexyl({[4,5-difluoro-2-({[(2,4,6-trimethylphenyl)amino]carbonyl}amino)phenyl]carbonyl}amino)ethanoate (0.116 g, 0.24 mmol) in 3 mL of THF:methanol:water/4:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.095 g (84% yield) of desired product as a white solid. ES MS m/z 474 (M+H).

### Example 231

### 1-({[4′-(Methyloxy)-3-({[(2,4,6-trimethylphenyl)amino]carbonyl}amino)-4-biphenylyl]carbonyl}amino)cyclooctanecarboxylic acid

### Step 1

### Methyl 1-({[3-nitro-4′-(methyloxy)-4-biphenylyl]carbonyl}amino)cyclooctanecarboxylate

HATU (0.524 g, 1.38 mmol) was added to a solution of 4′-(methyloxy)-3-nitro-4-biphenylcarboxylic acid (0.250 g, 0.92 mmol), methyl 2-amino-2-ethyloctanoate (0.169 g, 0.92 mmol), and diisopropylethylamine (0.24 mL, 1.38 mmol) in 10 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.276 (68% yield) g of desired product as a yellow solid.

### Step 2

### Methyl 1-({[3-amino-4′-(methyloxy)-4-biphenylyl]carbonyl}amino)cyclooctanecarboxylate

A mixture of methyl 1-({[3-amino-4′-(methyloxy)-4-biphenylyl]carbonyl}amino)cyclooctanecarboxylate (0.274 g, 0.62 mmol) and 5% palladium on carbon (0.066 g, 0.031 mmol) in 15 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated. The residue was purified by chromatography on silica gel with hexane/ethyl acetate to give 0.260 g of desired product as an off-white solid.

### Step 3

### Methyl 1-({[4′-(methyloxy)-3-({[(2,4,6-trimethylphenyl)amino]carbonyl}amino)-4-biphenylyl]carbonyl}amino)cyclooctanecarboxylate

2,4,6-Trimethylphenylisocyanate (0.304 g, 1.89 mmol) was added to a solution of methyl 1-({[3-amino-4′-(methyloxy)-4-biphenylyl]carbonyl}amino)cyclooctanecarboxylate (0.258 g, 0.63 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.238 g (66% yield) of desired product as a white solid.

### Step 4

### 1-({[4′-(Methyloxy)-3-({[(2,4,6-trimethylphenyl)amino]carbonyl}amino)-4-biphenylyl]carbonyl}amino)cyclooctanecarboxylic acid

Lithium hydroxide (0.094 g, 3.9 mmol) was added to a solution of methyl 1-({[4′-(methyloxy)-3-({[(2,4,6-trimethylphenyl)amino]carbonyl}amino)-4-biphenylyl]carbonyl}amino)cyclooctanecarboxylate (0.223 g, 0.39 mmol) in 6 mL of THF:methanol:water/4:1:1. The mixture was heated at 50° C. overnight. The solvent was evaporated and 1N aqueous hydrochloric acid was added to the residue. The resulting suspension was extracted with ethyl acetate, dried over anhydrous sodium sulfate and the solvent removed under vacuum to give 0.088 g (40% yield) of desired product as a white solid. ES MS m/z 558 (M+H).

### Example 232

### N-{[3-{[({2,6-Dichloro-4-[(trifluoromethyl)oxy]phenyl}amino)carbonyl]amino}-4′-(methyloxy)-4-biphenylyl]carbonyl}-O-(1,1-dimethylethyl)-L-threonine

### Step 1

### Methyl 4′-(methyloxy)-3-nitro-4-biphenylcarboxylate

Methyl 4-chloro-2-nitrobenzoate (1.00 g, 4.64 mmol), 4-methoxyphenylboronic acid (0.77 g, 5.10 mmol), trans-dichlorobis(tricyclohexylphosphine)palladium(II) (0.171 g, 0.23 mmol) and cesium fluoride (2.11 g, 13.9 mmol) were mixed in 13 mL of acetonitrile:water/3:1 in each of four microwave reaction vials and heated in a microwave reactor at 150° C. for 5 minutes. The cooled reaction mixtures were combined and filtered through Celite, diluted with ethyl acetate and washed with water and brine. The organic phase was dried over anhydrous sodium sulfate and the solvent was evaporated. Chromatography on silica gel with hexane/ethyl acetate gave 4.59 g (86% yield) of desired product.

### Step 2

### 4′-(Methyloxy)-3-nitro-4-biphenylcarboxylic acid

Lithium hydroxide (3.81 g, 158.8 mmol) was added to a solution of methyl 4′-(methyloxy)-3-nitro-4-biphenylcarboxylate (4.56 g, 15.98 mmol) in 50 ml of THF:methanol:water/3:1:1. The mixture was stirred at room temperature for 2.5 hours. The solvent was evaporated and the residue was treated with aqueous 1N hydrochloric acid, and extracted with ethyl acetate. The organic phase was dried over sodium sulfate and the solvent was evaporated to give 4.37 g (100% yield) of desired product as a yellow solid.

### Step 3

### Methyl O-(1,1-dimethylethyl)-N-{[4′-(methyloxy)-3-nitro-4-biphenylyl]carbonyl}-L-threoninate

HATU (7.07 g, 18.6 mmol) was added to a solution of 4′-(methyloxy)-3-nitro-4-biphenylcarboxylic acid (3.37 g, 12.4 mmol), methyl O-(1,1-dimethylethyl)-L-threoninate hydrochloride (2.79 g, 12.4 mmol), and diisopropylethylamine (3.2 mL, 18.6 mmol) in 100 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 3.79 (69% yield) g of desired product as a white solid.

### Step 4

### Methyl N-{[3-amino-4′-(methyloxy)-4-biphenylyl]carbonyl}-O-(1,1-dimethylethyl)-L-threoninate

A mixture of methyl O-(1,1-dimethylethyl)-N-{[4′-(methyloxy)-3-nitro-4-biphenylyl]carbonyl}-L-threoninate (3.77 g, 8.49 mmol) and 5% palladium on carbon (0.894 g, 0.42 mmol) in ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated. The residue was purified by chromatography on silica gel with hexane/ethyl acetate to give 3.38 g (96% yield) of desired product as an off-white solid.

### Step 5

### Methyl N-{[3-{[({2,6-dichloro-4-[(trifluoromethyl)oxy]phenyl}amino)carbonyl]amino}-4′-(methyloxy)-4-biphenylyl]carbonyl}-O-(1,1-dimethylethyl)-L-threoninate

1,3-Dichloro-2-isocyanato-5-[(trifluoromethyl)oxy]benzene (0.316 g, 1.21 mmol) was added to a solution of methyl N-{[3-amino-4′-(methyloxy)-4-biphenylyl]carbonyl}-O-(1,1-dimethylethyl)-L-threoninate (0.200 g, 0.48 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.243 g (74% yield) of desired product as a white solid.

### Step 6

### N-{[3-{[({2,6-Dichloro-4-[(trifluoromethyl)oxy]phenyl}amino)carbonyl]amino}-4′-(methyloxy)-4-biphenylyl]carbonyl}-O-(1,1-dimethylethyl)-L-threonine

Lithium hydroxide (0.085 g, 3.4 mmol) was added to a solution of methyl 1N-{[3-{[({2,6-dichloro-4-[(trifluoromethyl)oxy]phenyl}amino)carbonyl]amino}-4′-(methyloxy)-4-biphenylyl]carbonyl}-O-(1,1-dimethylethyl)-L-threoninate (0.236 g, 0.34 mmol) in 5 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.222 g (97% yield) of desired product as a white solid. ES MS m/z 672 (M+H).

### Example 233

### O-(1,1-Dimethylethyl)-N-{[3′-fluoro-3-({[(2,4,6-trimethylphenyl)amino]carbonyl}amino)-4-biphenylyl]carbonyl}-L-threonine

### Step 1

### Methyl 3′-fluoro-3-nitro-4-biphenylcarboxylate

A mixture methyl 4-chloro-2-nitrobenzoate (0.500 g, 2.32 mmol), 3-fluorophenylboronic acid (0.357 g, 2.55 mmol), trans-dichlorobis(tricyclohexylphosphine)palladium(II) (0.087 g, 0.116 mmol), cesium fluoride (1.06 g, 6.96 mmol), 1 mL of water and 6 mL of acetonitrile was heated in a microwave reactor at 150° C. for 5 minutes. The cooled reaction mixture was filtered through Celite, diluted with ethyl acetate, washed with water, and dried over sodium sulfate. Chromatography on silica gel with hexane/ethyl acetate gave 0.525 g (82% yield) of desired product as a white solid.

### Step 2

### 3′-Fluoro-3-nitro-4-biphenylcarboxylic acid

Lithium hydroxide (0.440 g, 18.3 mmol) was added to a solution of methyl 3′-fluoro-3-nitro-4-biphenylcarboxylate (0.504 g, 1.83 mmol) in 10 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.454 g (95% yield) of desired product as a white solid.

### Step 3

### Methyl O-(1,1-dimethylethyl)-N-[(3′-fluoro-3-nitro-4-biphenylyl)carbonyl]-L-threoninate

HATU (0.483 g, 1.27 mmol) was added to a solution of 3′-Fluoro-3-nitro-4-biphenylcarboxylic acid (0.221 g, 0.85 mmol), methyl O-(1,1-dimethylethyl)-L-threoninate hydrochloride (0.191 g, 0.85 mmol), and diisopropylethylamine (0.22 mL, 1.27 mmol) in 10 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.302 g (82% yield) g of desired product as a white solid.

### Step 4

### Methyl N-[(3-amino-3′-fluoro-4-biphenylyl)carbonyl]-O-(1,1-dimethylethyl)-L-threoninate

A mixture of methyl O-(1,1-dimethylethyl)-N-[(3′-fluoro-3-nitro-4-biphenylyl)carbonyl]-L-threoninate (0.293 g, 0.69 mmol) and 5% palladium on carbon (0.072 g, 0.034 mmol) in 25 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.264 g (95% yield) of desired product as a white solid.

### Step 5

### Methyl O-(1,1-dimethylethyl)-N-{[3′-fluoro-3-({[(2,4,6-trimethylphenyl)amino]carbonyl}amino)-4-biphenylyl]carbonyl}-L-threoninate

2,4,6-Trimethylphenylisocyanate (0.307 g, 1.91 mmol) was added to a solution of methyl N-[(3-amino-3′-fluoro-4-biphenylyl)carbonyl]-O-(1,1-dimethylethyl)-L-threoninate (0.256 g, 0.64 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.304 g (84% yield) of desired product as a white solid.

### Step 6

### O-(1,1-Dimethylethyl)-N-{[3′-fluoro-3-({[(2,4,6-trimethylphenyl)amino]carbonyl}amino)-4-biphenylyl]carbonyl}-L-threonine

Lithium hydroxide (0.124 g, 5.2 mmol) was added to a solution of methyl O-(1,1-dimethylethyl)-N-{[3′-fluoro-3-({[(2,4,6-trimethylphenyl)amino]carbonyl}amino)-4-biphenylyl]carbonyl}-L-threoninate (0.292 g, 0.52 mmol) in 5 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.260 g (91% yield) of desired product as a white solid. ES MS m/z 550 (M+H).

### Example 234

### (2S)-Cyclohexyl({[3′-fluoro-3-({[(2,4,6-trimethylphenyl)amino]carbonyl}amino)-4-biphenylyl]carbonyl}amino)ethanoic acid

### Step 1

### Methyl(2S)-cyclohexyl{[(3′-fluoro-3-nitro-4-biphenylyl)carbonyl]amino}ethanoate

HATU (0.471 g, 1.24 mmol) was added to a solution of 3′-Fluoro-3-nitro-4-biphenylcarboxylic acid (0.216 g, 0.83 mmol), methyl(2S)-amino(cyclohexyl)ethanoate hydrochloride (0.172 g, 0.83 mmol), and diisopropylethylamine (0.22 mL, 1.27 mmol) in 10 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.216 g (63% yield) of desired product as a white solid.

### Step 2

### Methyl(2S)-{[(3-amino-3′-fluoro-4-biphenylyl)carbonyl]amino}(cyclohexyl)ethanoate

A mixture of methyl(2S)-cyclohexyl{[(3′-fluoro-3-nitro-4-biphenylyl)carbonyl]amino}ethanoate (0.215 g, 0.52 mmol) and 5% palladium on carbon (0.055 g, 0.026 mmol) in ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.192 g (96% yield) of desired product as a light tan solid.

### Step 3

### Methyl(2S)-cyclohexyl({[3′-fluoro-3-({[(2,4,6-trimethylphenyl)amino]carbonyl}amino)-4-biphenylyl]carbonyl}amino)ethanoate

2,4,6-Trimethylphenylisocyanate (0.241 g, 1.5 mmol) was added to a solution of methyl(2S)-{[(3-amino-3′-fluoro-4-biphenylyl)carbonyl]amino}(cyclohexyl)ethanoate (0.192 g, 0.50 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.213 g (78% yield) of desired product as a white solid.

### Step 4

### (2S)-Cyclohexyl({[3′-fluoro-3-({[(2,4,6-trimethylphenyl)amino]carbonyl}amino)-4-biphenylyl]carbonyl}amino)ethanoic acid

Lithium hydroxide (0.090 g, 3.8 mmol) was added to a solution of methyl(2S)-cyclohexyl({[3′-fluoro-3-({[(2,4,6-trimethylphenyl)amino]carbonyl}amino)-4-biphenylyl]carbonyl}amino)ethanoate (0.205 g, 0.38 mmol) in 5 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent was removed under vacuum to give 0.136 g (67% yield) of desired product as a white solid. ES MS m/z 532 (M+H).

### Example 235

### O-(1,1-Dimethylethyl)-N-{[3′-fluoro-4′-(methyloxy)-3-({[(2,4,6-trimethylphenyl)amino]carbonyl}amino)-4-biphenylyl]carbonyl}-L-threonine

### Step 1

### Methyl 3′-fluoro-4′-(methyloxy)-3-nitro-4-biphenylcarboxylate

In each of two microwave reaction vials, a mixture of methyl 4-chloro-2-nitrobenzoate (1.00 g, 4.64 mmol), 3-fluoro-4-methoxyphenylboronic acid (0.87 g, 5.10 mmol), trans-dichlorobis(tricyclohexylphosphine)palladium(II) (0.171 g, 0.23 mmol), cesium fluoride (2.11 g, 13.9 mmol), 2 mL of water and 12 mL of acetonitrile was heated in a microwave reactor at 150° C. for 5 minutes. The cooled reaction mixtures were combined, diluted with ethyl acetate, filtered through Celite, washed with water and dried over sodium sulfate. Chromatography on silica gel with hexane/ethyl acetate gave 2.24 g (79% yield) of desired product as an off-white solid.

### Step 2

### 3′-Fluoro-4′-(methyloxy)-3-nitro-4-biphenylcarboxylic acid

Lithium hydroxide (0.53 g, 21.9 mmol) was added to a solution of methyl 3′-fluoro-4′-(methyloxy)-3-nitro-4-biphenylcarboxylate (2.23 g, 7.31 mmol) in 50 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 1.87 g (88% yield) of desired product as a white solid.

### Step 3

### Methyl O-(1,1-dimethylethyl)-N-{[3′-fluoro-4′-(methyloxy)-3-nitro-4-biphenylyl]carbonyl}-L-threoninate

HATU (0.585 g, 1.54 mmol) was added to a solution of 3′-Fluoro-4′-(methyloxy)-3-nitro-4-biphenylcarboxylic acid (0.300 g, 1.03 mmol), methyl O-(1,1-dimethylethyl)-L-threoninate hydrochloride (0.232 g, 1.03 mmol), and diisopropylethylamine (0.27 mL, 1.54 mmol) in 10 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.392 g (82% yield) g of desired product as a white solid.

### Step 4

### Methyl N-{[3-amino-3′-fluoro-4′-(methyloxy)-4-biphenylyl]carbonyl}-O-(1,1-dimethylethyl)-L-threoninate

A mixture of methyl O-(1,1-dimethylethyl)-N-{[3′-fluoro-4′-(methyloxy)-3-nitro-4-biphenylyl]carbonyl}-L-threoninate (0.388 g, 0.84 mmol) and 5% palladium on carbon (0.089 g, 0.042 mmol) in 25 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.340 g (94% yield) of desired product as an off-white solid.

### Step 5

### Methyl O-(1,1-dimethylethyl)-N-{[3′-fluoro-4′-(methyloxy)-3-({[(2,4,6-trimethylphenyl)amino]carbonyl}amino)-4-biphenylyl]carbonyl}-L-threoninate

2,4,6-Trimethylphenylisocyanate (0.376 g, 0.78 mmol) was added to a solution of methyl N-{[3-amino-3′-fluoro-4′-(methyloxy)-4-biphenylyl]carbonyl}-O-(1,1-dimethylethyl)-L-threoninate (0.336 g, 0.78 mmol) in 7 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl and saturated aqueous sodium bicarbonate, dried over anhydrous sodium sulfate and the solvent evaporated under reduced pressure. Chromatography on silica gel with hexane/ethyl acetate gave 0.359 g (60% yield) of desired product as a white solid.

### Step 6

### O-(1,I-Dimethylethyl)-N-{[3′-fluoro-4′-(methyloxy)-3-({[(2,4,6-trimethylphenyl)amino]carbonyl}amino)-4-biphenylyl]carbonyl}-L-threonine

Lithium hydroxide (0.144 g, 6.0 mmol) was added to a solution of methyl O-(1,1-dimethylethyl)-N-{[3′-fluoro-4′-(methyloxy)-3-({[(2,4,6-trimethylphenyl)amino]carbonyl}amino)-4-biphenylyl]carbonyl}-L-threoninate (0.357 g, 0.60 mmol) in 10 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.319 g (92% yield) of desired product as a white solid. ES MS m/z 580 (M+H).

### Example 236

### O-(1,1-Dimethylethyl)-N-{[3-({[(2,6-dimethyl-4-propylphenyl)amino]carbonyl}amino)-4′-(methyloxy)-4-biphenylyl]carbonyl}-L-threonine

### Step 1

### Methyl O-(1,1-dimethylethyl)-N-{[4′-(methyloxy)-3-nitro-4-biphenylyl]carbonyl}-L-threoninate

HATU (1.06 g, 2.79 mmol) was added to a solution of 4′-(methyloxy)-3-nitro-4-biphenylcarboxylic acid (0.509 g, 1.86 mmol), methyl O-(1,1-dimethylethyl)-L-threoninate hydrochloride (0.420 g, 1.86 mmol), and diisopropylethylamine (0.48 mL, 2.79 mmol) in 15 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.589 g (71% yield) g of desired product as a white solid.

### Step 2

### Methyl N-{[3-amino-4′-(methyloxy)-4-biphenylyl]carbonyl}-O-(1,1-dimethylethyl)-L-threoninate

A mixture of methyl O-(1,1-dimethylethyl)-N-{[4′-(methyloxy)-3-nitro-4-biphenylyl]carbonyl}-L-threoninate (0.581 g, 1.31 mmol) and 5% palladium on carbon (0.139 g, 0.065 mmol) in ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.521 g (96% yield) of desired product as a beige solid.

### Step 3

### Methyl N-{[3-({[(4-bromo-2,6-dimethylphenyl)amino]carbonyl}amino)-4′-(methyloxy)-4-biphenylyl]carbonyl}-O-(1,1-dimethylethyl)-L-threoninate

5-bromo-2-isocyanato-1,3-dimethylbenzene (0.205 g, 0.91 mmol) was added to a solution of N-{[3-amino-4′-(methyloxy)-4-biphenylyl]carbonyl}-O-(1,1-dimethylethyl)-L-threoninate (0.150 g, 0.36 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.226 g of desired product as a white solid.

### Step 4

### Methyl O-(1,1-dimethylethyl)-N-{[3-[({[2,6-dimethyl-4-(2-propen-1-yl)phenyl]amino}carbonyl)amino]-4′-(methyloxy)-4-biphenylyl]carbonyl}-L-threoninate

Tributyl(2-propen-1-yl)stannane (0.138 g, 0.41 mmol) was added to a suspension of methyl N-{[3-({[(4-bromo-2,6-dimethylphenyl)amino]carbonyl}amino)-4′-(methyloxy)-4-biphenylyl]carbonyl}-O-(1,1-dimethylethyl)-L-threoninate (0.226 g, 0.35 mmol) and tetrakis(triphenylphosphine)palladium(0) (0.024 g, 0.021 mmol) in 4.5 mL of acetonitrile. The mixture was heated to 150° C. in a microwave reactor for 30 minutes. The solvent was removed under vacuum and the residue was purified by chromatography on silica gel with hexane/ethyl acetate to give 0.166 g of a white solid containing about 75% of desired product. This material was carried on to the next step without further purification.

### Step 5

### Methyl O-(1,1-dimethylethyl)-N-{[3-({[(2,6-dimethyl-4-propylphenyl)amino]carbonyl}amino)-4′-(methyloxy)-4-biphenylyl]carbonyl}-L-threoninate

A mixture of methyl O-(1,1-dimethylethyl)-N-{[3-[({[2,6-dimethyl-4-(2-propen-1-yl)phenyl]amino}carbonyl)amino]-4′-(methyloxy)-4-biphenylyl]carbonyl}-L-threoninate (0.164 g, 0.27 mmol) and 5% palladium on carbon (0.058 g, 0.027 mmol) in 10 mL of ethyl acetate in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.153 g of a mixture containing 85% desired product.

### Step 6

### O-(1,1-Dimethylethyl)-N-{[3-({[(2,6-dimethyl-4-propylphenyl)amino]carbonyl}amino)-4′-(methyloxy)-4-biphenylyl]carbonyl}-L-threonine

Lithium hydroxide (0.061 g, 2.55 mmol) was added to a solution of methyl O-(1,1-dimethylethyl)-N-{[3-({[(2,6-dimethyl-4-propylphenyl)amino]carbonyl}amino)-4′-(methyloxy)-4-biphenylyl]carbonyl}-L-threoninate (0.154 g, 0.255 mmol) in 5 mL of THF:methanol:water/3:1:1. The mixture was stirred at room temperature overnight. The solvent was evaporated and IN aqueous hydrochloric acid was added to the residue. The resulting suspension was extracted with ethyl acetate, dried over anhydrous sodium sulfate and the solvent removed under vacuum. Chromatography on silica gel with hexane/ethyl acetate gave 0.089 g (59% yield) of desired product as a white solid. ES MS m/z 590 (M+H).

### Example 237

### (2S)-Cyclohexyl({[3′-fluoro-4′-(methyloxy)-3-({[(2,4,6-trimethylphenyl)amino]carbonyl}amino)-4-biphenylyl]carbonyl}amino)ethanoic acid

### Step 1

### Methyl(2S)-cyclohexyl({[3′-fluoro-4′-(methyloxy)-3-nitro-4-biphenylyl]carbonyl}amino)ethanoate

HATU (0.585 g, 1.54 mmol) was added to a solution of 3′-fluoro-4′-(methyloxy)-3-nitro-4-biphenylcarboxylic acid (0.300 g, 1.03 mmol), methyl(2S)-amino(cyclohexyl)ethanoate hydrochloride (0.214 g, 1.03 mmol), and diisopropylethylamine (0.27 mL, 1.54 mmol) in 10 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.395 g (86% yield) g of desired product as a white solid.

### Step 2

### Methyl(2S)-({[3-amino-3′-fluoro-4′-(methyloxy)-4-biphenylyl]carbonyl}amino)(cyclohexyl)ethanoate

A mixture of methyl(2S)-cyclohexyl({[3′-fluoro-4′-(methyloxy)-3-nitro-4-biphenylyl]carbonyl}amino)ethanoate (0.391 g, 0.88 mmol) and 5% palladium on carbon (0.094 g, 0.044 mmol) in ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.330 g (90% yield) of desired product as a beige solid.

### Step 3

### Methyl(2S)-cyclohexyl({[3′-fluoro-4′-(methyloxy)-3-({[(2,4,6-trimethylphenyl)amino]carbonyl}amino)-4-biphenylyl]carbonyl}amino)ethanoate

2,4,6-Trimethylphenylisocyanate (0.379 g, 2.35 mmol) was added to a solution of methyl(2S)-({[3-amino-3′-fluoro-4′-(methyloxy)-4-biphenylyl]carbonyl}amino)(cyclohexyl)ethanoate (0.325 g, 0.78 mmol) in 7 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.378 g (84% yield) of desired product as a white solid.

### Step 4

### (2S)-Cyclohexyl({[3′-fluoro-4′-(methyloxy)-3-({[(2,4,6-trimethylphenyl)amino]carbonyl}amino)-4-biphenylyl]carbonyl}amino)ethanoic acid

Lithium hydroxide (0.156 g, 6.5 mmol) was added to a solution of methyl(2S)-cyclohexyl({[3′-fluoro-4′-(methyloxy)-3-({[(2,4,6-trimethylphenyl)amino]carbonyl}amino)-4-biphenylyl]carbonyl}amino)ethanoate (0.375 g, 0.65 mmol) in 10 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum. Chromatography on silica gel with hexane/ethyl acetate gave 0.158 g (43% yield) of desired product as a white solid. APCI MS m/z 562 (M+H).

### Example 238

### 1-({[3′-Fluoro-4′-(methyloxy)-3-({[(2,4,6-trimethylphenyl)amino]carbonyl}amino)-4-biphenylyl]carbonyl}amino)cyclooctanecarboxylic acid

### Step 1

### Methyl 1-({[3′-fluoro-4′-(methyloxy)-3-nitro-4-biphenylyl]carbonyl}amino)cyclooctanecarboxylate

HATU (0.585 g, 1.54 mmol) was added to a solution of 3′-fluoro-4′-(methyloxy)-3-nitro-4-biphenylcarboxylic acid (0.300 g, 1.03 mmol), methyl 1-aminocyclooctanecarboxylate hydrochloride (0.228 g, 1.03 mmol), and diisopropylethylamine (0.27 mL, 1.54 mmol) in 10 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.243 g (51% yield) g of desired product as an off-white solid.

### Step 2

### Methyl 1-({[3-amino-3′-fluoro-4′-(methyloxy)-4-biphenylyl]carbonyl}amino)cyclooctanecarboxylate

A mixture of methyl 1-({[3′-fluoro-4′-(methyloxy)-3-nitro-4-biphenylyl]carbonyl}amino)cyclooctanecarboxylate (0.240 g, 0.52 mmol) and 5% palladium on carbon (0.056 g, 0.026 mmol) in 25 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.212 g (95% yield) of desired product as an off-white solid.

### Step 3

### Methyl 1-({[3′-fluoro-4′-(methyloxy)-3-({[(2,4,6-trimethylphenyl)amino]carbonyl}amino)-4-biphenylyl]carbonyl}amino)cyclooctanecarboxylate

2,4,6-Trimethylphenylisocyanate (0.232 g, 1.44 mmol) was added to a solution of methyl 1-({[3-amino-3′-fluoro-4′-(methyloxy)-4-biphenylyl]carbonyl}amino)cyclooctanecarboxylate (0.206 g, 0.48 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.214 g (76% yield) of desired product as a white solid.

### Step 4

### Methyl 1-({[3′-fluoro-4′-(methyloxy)-3-({[(2,4,6-trimethylphenyl)amino]carbonyl}amino)-4-biphenylyl]carbonyl}amino)cyclooctanecarboxylate

Lithium hydroxide (0.082 g, 3.4 mmol) was added to a solution of methyl 1-({[3′-fluoro-4′-(methyloxy)-3-({[(2,4,6-trimethylphenyl)amino]carbonyl}amino)-4-biphenylyl]carbonyl}amino)cyclooctanecarboxylate (0.201 g, 0.34 mmol) in 5 mL of THF:methanol:water/3:1:1. The mixture was heated at 50° C. overnight. The solvent was evaporated and 1N aqueous hydrochloric acid was added to the residue. The resulting suspension was extracted with ethyl acetate, dried over anhydrous sodium sulfate and the solvent removed under vacuum. Chromatography on silica gel with hexane/ethyl acetate gave 0.170 g (43% yield) of desired product as a white solid. ES MS m/z 574 (M−H).

### Example 239

### (2S)-[({3-[({[2,4-bis(methyloxy)phenyl]amino}carbonyl)amino]-2-naphthalenyl}carbonyl)amino](cyclohexyl)ethanoic acid

### Step 1

### Methyl(2S)-{[(3-amino-2-naphthalenyl)carbonyl]amino}(cyclohexyl)ethanoate

HATU (6.55 g, 17.23 mmol) was added to a solution of 3-amino-2-naphthalenecarboxylic acid (5.0 g, 14.35 mmol), methyl(2S)-amino(cyclohexyl)ethanoate hydrochloride (3.53 g, 17 mmol) and diisopropylethylamine (2.22 g, 17.21 mmol) in 100 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 5.01 g of light yellow solid.

### Step 2

### Methyl(2S)-[({3-[({[2,4-bis(methyloxy)phenyl]amino}carbonyl)amino]-2-naphthalenyl}carbonyl)amino](cyclohexyl)ethanoate

Methyl(2S)-[(3-amino-2-naphthoyl) amino](cyclohexyl)ethanoate (0.2 g, 0.588 mmol) in 3 mL of DMF was treated with triethylamine (0.16 g, 1.58 mmol) and 1-isocyanato-2,4-bis(methyloxy)benzene (0.13 g, 0.73 mmol) and was heated to 70° C. for ca. 15 h overnight. The reaction was quenched with 1N HCl and extracted with ethyl acetate. The organic layer was dried over magnesium sulfate, filtered, and the solvent evaporated. Chromatography on silica gel with hexane/ethyl acetate gave 0.035 g of product.

### Step 3

### (2S)-[({3-[({[2,4-bis(methyloxy)phenyl]amino}carbonyl)amino]-2-naphthalenyl}carbonyl)amino](cyclohexyl)ethanoic acid

Lithium hydroxide monohydrate (0.016 g, 0.67 mmol) was added to a solution of Methyl(2S)-[({3-[({[2,4-bis(methyloxy)phenyl]amino}carbonyl)amino]-2-naphthalenyl}carbonyl)amino](cyclohexyl)ethanoate (0.035 g, 0.067 mmol) in dioxane:water/10:1(5 ml). The mixture was stirred at RT overnight. The reaction mixture was acidified with 1N aqueous HCl and extracted with ethyl acetate. The organic phase was filtered through Varian chem-elut tubes and concentrated to dryness to give 6.3 mg (18% yield) of desired product as a light orange solid. ES MS m/z 506 (M+H).

### Example 240

### (2S)-[({3-[({[3,5-bis(trifluoromethyl)phenyl]amino}carbonyl)amino]-2-naphthalenyl}carbonyl)amino](cyclohexyl)ethanoic acid

### Step 1

### Methyl(2S)-[({3-[({[3,5-bis(trifluoromethyl)phenyl]amino}carbonyl)amino]-2-naphthalenyl}carbonyl)amino](cyclohexyl)ethanoate

Methyl(2S)-[(3-amino-2-naphthoyl) amino](cyclohexyl)ethanoate (0.2 g, 0.588 mmol) in 3 mL of DMF was treated with triethylamine (0.16 g, 1.58 mmol) and 1-isocyanato-3,5-bis(trifluoromethyl)benzene (0.18 g, 0.71 mmol) and was heated to 70° C. for ca. 15 h overnight. The reaction was quenched with 1N HCl and extracted with ethyl acetate. The organic layer was dried over magnesium sulfate, filtered, and the solvent evaporated. Chromatography on silica gel with hexane/ethyl acetate gave 0.264 g of product.

### Step 2

### (2S)-[({3-[({[3,5-bis(trifluoromethyl)phenyl]amino}carbonyl)amino]-2-naphthalenyl}carbonyl)amino](cyclohexyl)ethanoic acid

Lithium hydroxide monohydrate (0.11 g, 4.43 mmol) was added to a solution of Methyl(2S)-[({3-[(([3,5-bis(trifluoromethyl)phenyl]aminocarbonyl)amino]-2-naphthalenyl}carbonyl)amino](cyclohexyl)ethanoate (0.264 g, 0.44 mmol) in dioxane:water/10:1(5 ml). The mixture was stirred at RT overnight. The reaction mixture was acidified with 1N aqueous HCl and extracted with ethyl acetate. The organic phase was filtered through Varian chem-elut tubes and concentrated to dryness to give 103 mg (40% yield) of desired product as a light orange solid. ES MS m/z 582 (M+H).

### Example 241

### N-{[3-({[(2,6-dimethylphenyl)amino]carbonyl}amino)-2-naphthalenyl]carbonyl}-N-(2-pyridinylmethyl)glycine

### Step 1

### Ethyl N-[(3-amino-2-naphthalenyl)carbonyl]-N-(2-pyridinylmethyl)glycinate

HATU (0.27 g, 0.71 mmol) was added to a solution of 3-amino-2-naphthalenecarboxylic acid (0.2 g, 0.57 mmol), ethyl N-(2-pyridinylmethyl)glycinate hydrochloride (0.15 g, 77 mmol) and diisopropylethylamine (0.09 g, 0.70 mmol) in 3 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.311 g of amber oil.

### Step 2

### Ethyl N-{[3-({[(2,6-dimethylphenyl)amino]carbonyl}amino)-2-naphthalenyl]carbonyl}-N-(2-pyridinylmethyl)glycinate

Ethyl N-[(3-amino-2-naphthalenyl)carbonyl]-N-(2-pyridinylmethyl)glycinate (0.15 g, 0.413 mmol) in 3 mL of DMF was treated with triethylamine (0.087 g, 0.86 mmol) and 2-isocyanato-1,3-dimethylbenzene (0.067 g, 0.455 mmol) and was heated to 70° C. for ca. 3 h and then stirred at RT for 48 hours. The reaction was quenched with 1N HCl and extracted with ethyl acetate. The organic layer was dried over magnesium sulfate, filtered, and the solvent evaporated. Chromatography on silica gel with hexane/ethyl acetate gave 0.05 g of light yellow semi-solid.

### Step 3

### N-{[3-({[(2,6-dimethylphenyl)amino]carbonyl}amino)-2-naphthalenyl]carbonyl}-N-(2-pyridinylmethyl)glycine

Lithium hydroxide monohydrate (0.023 g, 0.96 mmol) was added to a solution of ethyl N-{[3-({[(2,6-dimethylphenyl)amino]carbonyl}amino)-2-naphthalenyl]carbonyl}-N-(2-pyridinylmethyl)glycinate (0.05 g, 0.098 mmol) in dioxane:water/10:1(5 ml). The mixture was stirred at RT overnight. The reaction mixture was acidified with 1N aqueous HCl and extracted with ethyl acetate. The organic phase was filtered through Varian tubes and concentrated to dryness to give 42 mg (89% yield) of desired product as a light orange solid. ES MS m/z 483 (M+H).

### Example 242

### N-(2-pyridinylmethyl)-N-{[3-({[(2,4,6-trichlorophenyl)amino]carbonyl}amino)-2-naphthalenyl]carbonyl}glycine

### Step 1

### ethyl N-(2-pyridinylmethyl)-N-{[3-({[(2,4,6-trichlorophenyl)amino]carbonyl}amino)-2-naphthalenyl]carbonyl}glycinate

Ethyl N-[(3-amino-2-naphthalenyl)carbonyl]-N-(2-pyridinylmethyl)glycinate (0.15 g, 0.413 mmol) in 3 mL of DMF was treated with triethylamine (0.087 g, 0.86 mmol) and 1,3,5-trichloro-2-isocyanatobenzene (0.100 g, 0.449 mmol) and was heated to 70° C. for ca. 3 h and then stirred at RT for 48 hours. The reaction was quenched with 1N HCl and extracted with ethyl acetate. The organic layer was dried over magnesium sulfate, filtered, and the solvent evaporated. Chromatography on silica gel with hexane/ethyl acetate gave 0.077 g of product.

### Step 2

### N-(2-pyridinylmethyl)-N-{[3-({[(2,4,6-trichlorophenyl)amino]carbonyl}amino)-2-naphthalenyl]carbonyl}glycine

Lithium hydroxide monohydrate (0.031 g, 1.29 mmol) was added to a solution of ethyl N-(2-pyridinylmethyl)-N-{[3-({[(2,4,6-trichlorophenyl)amino]carbonyl}amino)-2-naphthalenyl]carbonyl}glycinate (0.077 g, 0.131 mmol) in dioxane:water/10:1 (5 ml). The mixture was stirred at RT overnight. The reaction mixture was acidified with 1N aqueous HCl and extracted with ethyl acetate. The organic phase was filtered through Varian tubes and concentrated to dryness to give 65 mg (89% yield) of desired product as a cream solid. ES MS m/z 557 (M+H).

### Example 243

### N-(cyclohexylmethyl)-N-{[3-({[(2,6-dimethylphenyl)amino]carbonyl}amino)-2-naphthalenyl]carbonyl}glycine

### Step 1

### Phenylmethyl N-(cyclohexylmethyl)glycinate

Triethylamine (5.02 g, 49.65 mmol) was added to a solution of phenylmethyl glycinate hydrochloride (5.0 g, 24.81 mmol) in 15 ml of MeOH to which was then added cyclohexanecarbaldehyde (2.79 g, 24.93 mmol). The reaction was stirred at RT for 2 hours and then sodium borohydride (1.89 g, 49.96 mmol) was added portionwise, and the reaction was then allowed to stir at RT for 15 hours. The reaction was quenched with 5% sodium bicarbonate solution and diluted with ethyl acetate. The organic layer was dried over magnesium sulfate, filtered, and the solvent evaporated. Chromatography on silica gel with hexane/ethyl acetate gave 0.202 g of clear oil.

### Step 2

### Phenylmethyl N-[(3-amino-2-naphthalenyl)carbonyl]-N-(cyclohexylmethyl)glycinate

HATU (0.27 g, 0.71 mmol) was added to a solution of 3-amino-2-naphthalenecarboxylic acid (0.2 g, 0.57 mmol), phenylmethyl N-(cyclohexylmethyl)glycinate (0.18 g, 0.689 mmol) and diisopropylethylamine (0.089 g, 0.69 mmol) in 3 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.159 g of product.

### Step 3

### Phenylmethyl N-(cyclohexylmethyl)-N-{[3-({[(2,6-dimethylphenyl)amino]carbonyl}amino)-2-naphthalenyl]carbonyl}glycinate

Phenylmethyl N-[(3-amino-2-naphthalenyl)carbonyl]-N-(cyclohexylmethyl)glycinate (0.159 g, 0.369 mmol) in 3 mL of DMF was treated with triethylamine (0.074 g, 0.73 mmol) and 2-isocyanato-1,3-dimethylbenzene (0.59 g, 4.01 mmol) and was heated to 70° C. for ca. 15 hours. The reaction was quenched with 1N HCl and extracted with ethyl acetate. The organic layer was dried over magnesium sulfate, filtered, and the solvent evaporated. Chromatography on silica gel with hexane/ethyl acetate gave 0.072 g of product.

### Step 4

### N-(cyclohexylmethyl)-N-{[3-({[(2,6-dimethylphenyl)amino]carbonyl}amino)-2-naphthalenyl]carbonyl}glycine

Palladium (10% weight on activated carbon, catalytic amount) was added to a solution of phenylmethyl N-(cyclohexylmethyl)-N-{[3-({[(2,6-dimethylphenyl)amino]carbonyl}amino)-2-naphthalenyl]carbonyl}glycinate (0.072 g, 0.125 mmol) in 5 ml of EtOH in a flask under nitrogen. A balloon of H₂ was then affixed to the reaction flask and the reaction was stirred for 2 hours at RT. The reaction was then filtered through a pad of Celite and the solvent evaporated to give 0.023 g (38% yield) of product as a cream solid ES MS m/z 488 (M+H).

### Example 244

### N-cyclopentyl-N-{[3-({[(2,6-dimethylphenyl)amino]carbonyl}amino)-2-naphthalenyl]carbonyl}glycine

### Step 1

### Phenylmethyl N-cyclopentylglycinate

Triethylamine (5.02 g, 49.65 mmol) was added to a solution of phenylmethyl glycinate hydrochloride (5.0 g, 24.81 mmol) in 15 ml of MeOH to which was then added cyclopentanone (2.74 g, 32.53 mmol). The reaction was stirred at RT for 2 hours and then sodium borohydride (1.89 g, 49.96 mmol) was added portionwise, and the reaction was then allowed to stir at RT for 15 hours. The reaction was quenched with 5% sodium bicarbonate solution and diluted with ethyl acetate. The organic layer was dried over magnesium sulfate, filtered, and the solvent evaporated. Chromatography on silica gel with hexane/ethyl acetate gave 0.363 g of clear oil.

### Step 2

### Phenylmethyl N-[(3-amino-2-naphthalenyl)carbonyl]-N-cyclopentylglycinate

HATU (0.27 g, 0.71 mmol) was added to a solution of 3-amino-2-naphthalenecarboxylic acid (0.2 g, 0.57 mmol), phenylmethyl N-cyclopentylglycinate (0.16 g, 0.686 mmol) and diisopropylethylamine (0.089 g, 0.69 mmol) in 3 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.166 g of product.

### Step 3

### Phenylmethyl N-cyclopentyl-N-{[3-({[(2,6-dimethylphenyl)amino]carbonyl}amino)-2-naphthalenyl]carbonyl}glycinate

Phenylmethyl N-[(3-amino-2-naphthalenyl)carbonyl]-N-cyclopentylglycinate (0.166 g, 0.412 mmol) in 3 mL of DMF was treated with triethylamine (0.074 g, 0.73 mmol) and 2-isocyanato-1,3-dimethylbenzene (0.59 g, 4.01 mmol) and was heated to 70° C. for ca. 15 hours. The reaction was quenched with 1N HCl and extracted with ethyl acetate. The organic layer was dried over magnesium sulfate, filtered, and the solvent evaporated. Chromatography on silica gel with hexane/ethyl acetate gave 0.092 g of product.

### Step 4

### N-cyclopentyl-N-{[3-({[(2,6-dimethylphenyl)amino]carbonyl}amino)-2-naphthalenyl]carbonyl}glycine

Palladium (10% weight on activated carbon, catalytic amount) was added to a solution of phenylmethyl N-(cyclohexylmethyl)-N-{[3-({[(2,6-dimethylphenyl)amino]carbonyl}amino)-2-naphthalenyl]carbonyl}glycinate (0.072 g, 0.125 mmol) in 5 ml of EtOH in a flask under nitrogen. A balloon of H₂ was then affixed to the reaction flask and the reaction was stirred for 2 hours at RT. The reaction was then filtered through a pad of Celite and the solvent evaporated to give 0.021 g (27% yield) of product as a cream solid ES MS m/z 460 (M+H).

### Example 245

### N-cyclohexyl-N-{[3-({[(2,6-dimethylphenyl)amino]carbonyl}amino)-2-naphthalenyl]carbonyl}glycine

### Step 1

### Phenylmethyl N-cyclohexylglycinate

Triethylamine (5.02 g, 49.65 mmol) was added to a solution of phenylmethyl glycinate hydrochloride (5.0 g, 24.81 mmol) in 15 ml of MeOH to which was then added cyclohexanone (2.45 g, 24.96 mmol). The reaction was stirred at RT for 2 hours and then sodium borohydride (1.89 g, 49.96 mmol) was added portionwise, and the reaction was then allowed to stir at RT for 15 hours. The reaction was quenched with 5% sodium bicarbonate solution and diluted with ethyl acetate. The organic layer was dried over magnesium sulfate, filtered, and the solvent evaporated. Chromatography on silica gel with hexane/ethyl acetate gave 3.65 g of light amber oil.

### Step 2

### Phenylmethyl N-[(3-amino-2-naphthalenyl)carbonyl]-N-cyclohexylglycinate

HATU (2.28 g, 5.99 mmol) was added to a solution of 3-amino-2-naphthalenecarboxylic acid (1.0 g, 5.34 mmol), phenylmethyl N-cyclohexylglycinate (1.59 g, 6.43 mmol) and diisopropylethylamine (0.78 g, 6.02 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.240 g of product.

### Step 3

### Phenylmethyl N-cyclohexyl-N-{[3-({[(2,6-dimethylphenyl)amino]carbonyl}amino)-2-naphthalenyl]carbonyl}glycinate

Phenylmethyl N-[(3-amino-2-naphthalenyl)carbonyl]-N-cyclohexylglycinate (0.240 g, 0.576 mmol) in 10 mL of DMF was treated with triethylamine (0.12 g, 1.15 mmol) and 2-isocyanato-1,3-dimethylbenzene (0.09 g, 0.61 mmol) and was heated to 70° C. for ca. 48 hours. The reaction was quenched with 1N HCl and extracted with ethyl acetate. The organic layer was dried over magnesium sulfate, filtered, and the solvent evaporated. Chromatography on silica gel with hexane/ethyl acetate gave 0.077 g of product.

### Step 4

### N-cyclohexyl-N-{[3-({[(2,6-dimethylphenyl)amino]carbonyl}amino)-2-naphthalenyl]carbonyl}glycine

Palladium (10% weight on activated carbon, catalytic amount) was added to a solution of Phenylmethyl N-cyclohexyl-N-{[3-({[(2,6-dimethylphenyl)amino]carbonyl}amino)-2-naphthalenyl]carbonyl}glycinate (0.077 g, 0.137 mmol) in 5 ml of EtOH in a flask under nitrogen. A balloon of H₂ was then affixed to the reaction flask and the reaction was stirred for 2 hours at RT. The reaction was then filtered through a pad of Celite and the solvent evaporated to give 0.011 g (17% yield) of product as a cream solid ES MS m/z 474 (M+H).

### Example 246

### 2,2′-({[3-({[(2,6-dimethylphenyl)amino]carbonyl}amino)-2-naphthalenyl]carbonyl}imino)diacetic acid

### Step 1

### Bis(1,1-dimethylethyl) 2,2′-{[(3-amino-2-naphthalenyl)carbonyl]imino}diacetate

HATU (2.44 g, 6.41 mmol) was added to a solution of 3-amino-2-naphthalenecarboxylic acid (1.0 g, 5.34 mmol), bis(1,1-dimethylethyl) 2,2′-iminodiacetate (1.57 g, 6.40 mmol) and diisopropylethylamine (0.83 g, 6.42 mmol) in 10 mL of DMF. The mixture was stirred at RT for ca. 48 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 1.66 g of product as a light orange solid.

### Step 2

### Bis(1,1-dimethylethyl) 2,2′-({[3-({[(2,6-dimethylphenyl)amino]carbonyl}amino)-2-naphthalenyl]carbonyl}imino)diacetate

Bis(1,1-dimethylethyl) 2,2′-{[(3-amino-2-naphthalenyl)carbonyl]imino}diacetate (1.66 g, 4.00 mmol) in 50 mL of DMF was treated with triethylamine (0.81 g, 8.03 mmol) and 2-isocyanato-1,3-dimethylbenzene (0.65 g, 4.42 mmol) and was heated to 70° C. for ca. 15 hours. The reaction was quenched with 1N HCl and extracted with ethyl acetate. The organic layer was dried over magnesium sulfate, filtered, and the solvent evaporated to give 1.41 g of crude product. Chromatography on silica gel of 1.0 g of crude product with hexane/ethyl acetate gave 0.44 g of product as a fluffy yellow semi-solid.

### Step 3

### 2,2′-({[3-({[(2,6-dimethylphenyl)amino]carbonyl}amino)-2-naphthalenyl]carbonyl}imino)diacetic acid

Bis(1,I-dimethylethyl) 2,2′-({[3-({[(2,6-dimethylphenyl)amino]carbonyl}amino)-2-naphthalenyl]carbonyl}imino)diacetate (0.44 g, 0.78 mmol) was dissolved in 2:1 (v/v) of TFA (2 ml) and CH₂Cl₂ (1 ml) and stirred at RT for 30 minutes. The reaction was concentrated by a nitrogen stream and dried further in vacuo to give 252 mg (72% yield) of product as a fluffy amber solid. ES MS m/z 450 (M+H).

### Example 247

### (2S)-({[3-({[(4-butylphenyl)amino]carbonyl}amino)-2-naphthalenyl]carbonyl}amino)(cyclohexyl)ethanoic acid

### Step 1

### Methyl(2S)-({[3-({[(4-butylphenyl)amino]carbonyl}amino)-2-naphthalenyl]carbonyl}amino)(cyclohexyl)ethanoate

Methyl(2S)-{[(3-amino-2-naphthalenyl)carbonyl]amino}(cyclohexyl)ethanoate (0.2 g, 0.588 mmol) in 3 mL of DMF was treated with triethylamine (0.12 g, 1.15 mmol) and 1-butyl-4-isocyanatobenzene (0.12 g, 0.685 mmol) and was heated to 70° C. for ca. 3 hours and then allowed to stir at RT for ca. 15 hours. The reaction was quenched with 1N HCl and extracted with ethyl acetate. The organic layer was dried over magnesium sulfate, filtered, and the solvent evaporated. The crude material was then triturated with ethyl acetate and then filtered to give 0.135 g of product.

### Step 2

### (2S)-({[3-({[(4-butylphenyl)amino]carbonyl}amino)-2-naphthalenyl]carbonyl}amino)(cyclohexyl)ethanoic acid

Lithium hydroxide monohydrate (0.063 g, 2.61 mmol) was added to a solution of methyl(2S)-({[3-({[(4-butylphenyl)amino]carbonyl}amino)-2-naphthalenyl]carbonyl}amino)(cyclohexyl)ethanoate (0.135 g, 0.262 mmol) in dioxane:water/10:1 (3 ml). The mixture was stirred at RT overnight. The reaction mixture was acidified with 1N aqueous HCl and extracted with ethyl acetate. The organic phase was filtered through Varian Chem-elut tubes and concentrated to dryness and purified by Agilent prep-HPLC to give 11.2 mg (8% yield) of desired product as a cream solid. ES MS m/z 502 (M+H).

### Example 248

### (2S)-cyclohexyl{[(3-{[(2,3-dihydro-1-benzofuran-5-ylamino)carbonyl]amino}-2-naphthalenyl)carbonyl]amino}ethanoic acid

### Step 1

### Methyl(2S)-cyclohexyl{[(3-{[(2,3-dihydro-1-benzofuran-5-ylamino)carbonyl]amino}-2-naphthalenyl)carbonyl]amino}ethanoate

Methyl(2S)-{[(3-amino-2-naphthalenyl)carbonyl]amino}(cyclohexyl)ethanoate (0.2 g, 0.588 mmol) in 3 mL of DMF was treated with triethylamine (0.12 g, 1.15 mmol) and 5-isocyanato-2,3-dihydro-1-benzofuran (0.11 g, 0.682 mmol) and was heated to 70° C. for ca. 3 hours and then allowed to stir at RT for ca. 15 hours. The reaction was quenched with 1N HCl and extracted with ethyl acetate. The organic layer was dried over magnesium sulfate, filtered, and the solvent evaporated. The crude material was then triturated with ethyl acetate and then filtered to give 0.192 g of product.

### Step 2

### (2S)-cyclohexyl{[(3-{[(2,3-dihydro-1-benzofuran-5-ylamino)carbonyl]amino}-2-naphthalenyl)carbonyl]amino}ethanoic acid

Lithium hydroxide monohydrate (0.092 g, 3.83 mmol) was added to a solution of methyl(2S)-cyclohexyl{[(3-{[(2,3-dihydro-1-benzofuran-5-ylamino)carbonyl]amino}-2-naphthalenyl)carbonyl]amino}ethanoate (0.192 g, 0.383 mmol) in dioxane:water/10:1 (3 ml). The mixture was stirred at RT overnight. The reaction mixture was acidified with 1N aqueous HCl and extracted with ethyl acetate. The organic phase was filtered through Varian Chem-elut tubes and concentrated to dryness to give 98 mg (53% yield) of desired product as a tan solid. ES MS m/z 488 (M+H).

### Example 249

### (2S)-cyclohexyl({[3-({[(5-methyl-3-phenyl-4-isoxazolyl)amino]carbonyl}amino)-2-naphthalenyl]carbonyl}amino)ethanoic acid

### Step 1

### Methyl(2S)-cyclohexyl({[3-({[(5-methyl-3-phenyl-4-isoxazolyl)amino]carbonyl}amino)-2-naphthalenyl]carbonyl}amino)ethanoate

Methyl(2S)-{[(3-amino-2-naphthalenyl)carbonyl]amino}(cyclohexyl)ethanoate (0.2 g, 0.588 mmol) in 3 mL of DMF was treated with triethylamine (0.12 g, 1.15 mmol) and 4-isocyanato-5-methyl-3-phenylisoxazole (0.14 g, 0.699 mmol) and was heated to 70° C. for ca. 3 hours and then allowed to stir at RT for ca. 15 hours. The reaction was quenched with 1N HCl and extracted with ethyl acetate. The organic layer was dried over magnesium sulfate, filtered, and the solvent evaporated. The crude material was then chromatographed with ethyl acetate/hexanes to give 0.168 g of product.

### Step 2

### (2S)-cyclohexyl({[3-({[(5-methyl-3-phenyl-4-isoxazolyl)amino]carbonyl}amino)-2-naphthalenyl]carbonyl}amino)ethanoic acid

Lithium hydroxide monohydrate (0.075 g, 3.14 mmol) was added to a solution of methyl(2S)-cyclohexyl({[3-({[(5-methyl-3-phenyl-4-isoxazolyl)amino]carbonyl}amino)-2-naphthalenyl]carbonyl}amino)ethanoate (0.168 g, 0.311 mmol) in dioxane:water/10:1 (3 ml). The mixture was stirred at RT overnight. The reaction mixture was acidified with 1N aqueous HCl and extracted with ethyl acetate. The organic phase was dried over MgSO4, filtered and concentrated in vacuo to give 66 mg (42% yield) of product. ES MS m/z 527 (M+H).

### Example 250

### N-{[3-({[(2,6-dichlorophenyl)amino]carbonyl}amino)-2-naphthalenyl]carbonyl}-N-(phenylmethyl)-L-alanine

### Step 1

### Methyl N-[(3-amino-2-naphthalenyl)carbonyl]-N-(phenylmethyl)-L-alaninate

HATU (0.61 g, 1.60 mmol) was added to a solution of 3-amino-2-naphthalenecarboxylic acid (0.25 g, 1.34 mmol), methyl N-(phenylmethyl)-L-alaninate hydrochloride (0.37 g, 1.61 mmol) and diisopropylethylamine (0.21 g, 1.61 mmol) in 3 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.085 g of product.

### Step 2

### Methyl N-{[3-({[(2,6-dichlorophenyl)amino]carbonyl}amino)-2-naphthalenyl]carbonyl}-N-(phenylmethyl)-L-alaninate

Methyl N-[(3-amino-2-naphthalenyl)carbonyl]-N-(phenylmethyl)-L-alaninate (0.085 g, 0.235 mmol) in 3 mL of DMF was treated with triethylamine (0.047 g, 0.466 mmol) and 1,3-dichloro-2-isocyanatobenzene (0.049 g, 0.261 mmol) and was heated to 70° C. for ca. 3 hours and then allowed to stir at RT for ca. 48 hours. The reaction was quenched with 1N HCl and extracted with ethyl acetate and then filtered through a Varian Chem-elut tube. Chromatography on silica gel with hexane/ethyl acetate gave 0.077 g of product.

### Step 3

### N-{[3-({[(2,6-dichlorophenyl)amino]carbonyl}amino)-2-naphthalenyl]carbonyl}-N-(phenylmethyl)-L-alanine

Lithium hydroxide monohydrate (0.034 g, 1.42 mmol) was added to a solution of methyl N-{[3-({[(2,6-dichlorophenyl)amino]carbonyl}amino)-2-naphthalenyl]carbonyl}-N-(phenylmethyl)-L-alaninate (0.077 g, 0.140 mmol) in dioxane:water/10:1 (3 ml). The mixture was stirred at RT overnight. The reaction mixture was acidified with 1N aqueous HCl and extracted with ethyl acetate. The organic phase was dried over MgSO4, filtered and concentrated in vacuo, followed by purification on Agilent prep-HPLC to give 12.8 mg (16% yield) of product. ES MS m/z 536 (M+H).

### Example 251

### N-{[3-({[(2,6-dichlorophenyl)amino]carbonyl}amino)-2-naphthalenyl]carbonyl}-N-(phenylmethyl)phenylalanine

### Step 1

### Methyl N-[(3-amino-2-naphthalenyl)carbonyl]-N-(phenylmethyl)phenylalaninate

HATU (0.61 g, 1.60 mmol) was added to a solution of 3-amino-2-naphthalenecarboxylic acid (0.25 g, 1.34 mmol), methyl N-(phenylmethyl)phenylalaninate hydrochloride (0.49 g, 1.60 mmol) and diisopropylethylamine (0.21 g, 1.61 mmol) in 3 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.32 g of product.

### Step 2

### Methyl N-{[3-({[(2,6-dichlorophenyl)amino]carbonyl}amino)-2-naphthalenyl]carbonyl}-N-(phenylmethyl)phenylalaninate

Methyl N-[(3-amino-2-naphthalenyl)carbonyl]-N-(phenylmethyl)phenylalaninate (0.32 g, 0.730 mmol) in 3 mL of DMF was treated with triethylamine (0.145 g, 1.43 mmol) and 1,3-dichloro-2-isocyanatobenzene (0.15 g, 0.798 mmol) and was heated to 70° C. for ca. 3 hours and then allowed to stir at RT for ca. 48 hours. The reaction was quenched with 1N HCl and extracted with ethyl acetate and then filtered through a Varian Chem-elut tube. Chromatography on silica gel with hexane/ethyl acetate gave 0.030 g of product.

### Step 3

### N-{[3-({[(2,6-dichlorophenyl)amino]carbonyl}amino)-2-naphthalenyl]carbonyl}-N-(phenylmethyl)phenylalanine

Lithium hydroxide monohydrate (0.011 g, 0.459 mmol) was added to a solution of methyl N-{[3-({[(2,6-dichlorophenyl)amino]carbonyl}amino)-2-naphthalenyl]carbonyl}-N-(phenylmethyl)phenylalaninate (0.030 g, 0.0479 mmol) in dioxane:water/10:1 (3 ml). The mixture was stirred at RT overnight. The reaction mixture was acidified with 1N aqueous HCl and extracted with ethyl acetate. The organic phase was dried over magnesium sulfate, filtered and concentrated in vacuo to give 10.4 mg (32% yield) of product. ES MS m/z 611 (M−H).

### Example 252

### N-butyl-N-{[3-({[(2,6-dichlorophenyl)amino]carbonyl}amino)-2-naphthalenyl]carbonyl}glycine

### Step 1

### Phenylmethyl N-butylglycinate

Triethylamine (5.01 g, 49.50 mmol) was added to a solution of phenylmethyl glycinate hydrochloride (5.0 g, 24.81 mmol) in 100 ml of MeOH to which was then added butanal (1.80 g, 24.93 mmol). The reaction was stirred at RT for 2 hours and then sodium borohydride (1.89 g, 49.96 mmol) was added portionwise, and the reaction was then allowed to stir at RT for 15 hours. The reaction was quenched with 5% sodium bicarbonate solution and diluted with ethyl acetate. The organic layer was dried over magnesium sulfate, filtered, and the solvent evaporated. Chromatography on silica gel with hexane/ethyl acetate gave 0.454 g of clear oil.

### Step 2

### Phenylmethyl N-[(3-amino-2-naphthalenyl)carbonyl]-N-butylglycinate

HATU (0.61 g, 1.60 mmol) was added to a solution of 3-amino-2-naphthalenecarboxylic acid (0.25 g, 1.34 mmol), phenylmethyl N-butylglycinate (0.35 g, 1.58 mmol) and diisopropylethylamine (0.21 g, 1.61 mmol) in 3 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.155 g of product.

### Step 3

### Phenylmethyl N-butyl-N-{[3-({[(2,6-dichlorophenyl)amino]carbonyl}amino)-2-naphthalenyl]carbonyl}glycinate

Phenylmethyl N-[(3-amino-2-naphthalenyl)carbonyl]-N-butylglycinate (0.155 g, 0.397 mmol) in 3 mL of DMF was treated with triethylamine (0.080 g, 0.789 mmol) and 1,3-dichloro-2-isocyanatobenzene (0.082 g, 0.436 mmol) and was heated to 70° C. for ca. 3 hours and then allowed to stir at RT for ca. 48 hours. The reaction was quenched with 1N HCl and extracted with ethyl acetate and then filtered through a Varian Chem-elut tube: Chromatography on silica gel with hexane/ethyl acetate gave 0.0725 g of product.

### Step 4

### N-butyl-N-{[3-({[(2,6-dichlorophenyl)amino]carbonyl}amino)-2-naphthalenyl]carbonyl}glycine

Palladium (10% weight on activated carbon, catalytic amount) was added to a solution of phenylmethyl N-butyl-N-{[3-({[(2,6-dichlorophenyl)amino]carbonyl}amino)-2-naphthalenyl]carbonyl}glycinate (0.0725 g, 0.125 mmol) in 5 ml of EtOH in a flask under nitrogen. A balloon of H₂ was then affixed to the reaction flask and the reaction was stirred for 2 hours at RT. The reaction was then filtered through a pad of Celite and the solvent evaporated and then crude material purified by Agilent prep-HPLC to give 0.0235 g (38% yield) of product as a cream solid ES MS m/z 488 (M+H).

### Example 253

### N-{[3-({[(2,4,6-trimethylphenyl)amino]carbonyl}amino)-2-naphthalenyl]carbonyl}-L-norleucine

### Step 1

### Methyl N-[(3-amino-2-naphthalenyl)carbonyl]-L-norleucinate

HATU (1.14 g, 3.00 mmol) was added to a solution of 3-amino-2-naphthalenecarboxylic acid (0.5 g, 2.67 mmol), methyl L-norleucinate (0.47 g, 3.24 mmol) and diisopropylethylamine (0.38 g, 2.95 mmol) in 15 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 1.0 g of product as a yellow oil.

### Step 2

### Methyl N-{[3-({[(2,4,6-trimethylphenyl)amino]carbonyl}amino)-2-naphthalenyl]carbonyl}-L-norleucinate

Methyl N-[(3-amino-2-naphthalenyl)carbonyl]-L-norleucinate (1.0 g, 3.18 mmol) in 10 mL of pyridine was treated with 2-isocyanato-1,3,5-trimethylbenzene (2.6 g, 16.13 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.5 g of product as a white solid.

### Step 3

### N-{[3-({[(2,4,6-trimethylphenyl)amino]carbonyl}amino)-2-naphthalenyl]carbonyl}-L-norleucine

Lithium hydroxide monohydrate (0.25 g, 10.44 mmol) was added to a solution of methyl N-{[3-({[(2,4,6-trimethylphenyl)amino]carbonyl}amino)-2-naphthalenyl]carbonyl}-L-norleucinate (0.5 g, 1.05 mmol) in dioxane:water/10:1 (20 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.47 g (92% yield) of product. ES MS m/z 462 (M+H).

### Example 254

### (2S)-4-{[(1,1-dimethylethyl)oxy]carbonyl}-1-{[3-({[(2,4,6-trimethylphenyl)amino]carbonyl}amino)-2-naphthalenyl]carbonyl}-2-piperazinecarboxylic acid

### Step 1

### 1-(1,1-dimethylethyl) 3-methyl(3S)-4-[(3-amino-2-naphthalenyl)carbonyl]-1,3-piperazinedicarboxylate

HATU (1.14 g, 3.00 mmol) was added to a solution of 3-amino-2-naphthalenecarboxylic acid (0.5 g, 2.67 mmol), 1-(1,1-dimethylethyl) 3-methyl(3S)-1,3-piperazinedicarboxylate (0.78 g, 3.19 mmol) and diisopropylethylamine (0.38 g, 2.95 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.58 g of product as a tan solid.

### Step 2

### 1-(1,1-dimethylethyl) 3-methyl(3S)-4-{[3-({[(2,4,6-trimethylphenyl)amino]carbonyl}amino)-2-naphthalenyl]carbonyl}-1,3-piperazinedicarboxylate

1-(1,1-dimethylethyl) 3-methyl(3S)-4-[(3-amino-2-naphthalenyl)carbonyl]-1,3-piperazinedicarboxylate (0.58 g, 1.40 mmol) in 10 mL of pyridine was treated with 2-isocyanato-1,3,5-trimethylbenzene (1.13 g, 7.01 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.74 g of product as a light yellow semi-solid.

### Step 3

### (2S)-4-{[(1,1-dimethylethyl)oxy]carbonyl}-1-{[3-({[(2,4,6-trimethylphenyl)amino]carbonyl}amino)-2-naphthalenyl]carbonyl}-2-piperazinecarboxylic acid

Lithium hydroxide monohydrate (0.08 g, 3.34 mmol) was added to a solution of 1-(1,1-dimethylethyl) 3-methyl(3S)-4-{[3-({[(2,4,6-trimethylphenyl)amino]carbonyl}amino)-2-naphthalenyl]carbonyl}-1,3-piperazinedicarboxylate (0.2 g, 0.348 mmol) in dioxane:water/10:1 (4 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.144 g (73% yield) of product. ES MS m/z 561 (M+H).

### Example 255

### (2S)-cyclohexyl({[3-({[5-(2,4-dichlorophenyl)-2-furanyl]carbonyl}amino)-2-naphthalenyl]carbonyl}amino)ethanoic acid

### Step 1

### Methyl(2S)-{[(3-amino-2-naphthalenyl)carbonyl]amino}(cyclohexyl)ethanoate

HATU (6.55 g, 17.23 mmol) was added to a solution of 3-amino-2-naphthalenecarboxylic acid (5.0 g, 14.35 mmol), methyl(2S)-amino(cyclohexyl)ethanoate hydrochloride (3.53 g, 17 mmol) and diisopropylethylamine (2.22 g, 17.21 mmol) in 100 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 2.82 g of amber oil.

### Step 2

### Methyl(2S)-cyclohexyl({[3-({[5-(2,4-dichlorophenyl)-2-furanyl]carbonyl}amino)-2-naphthalenyl]carbonyl}amino)ethanoate

To a solution of methyl(2S)-{[(3-amino-2-naphthalenyl)carbonyl]amino}(cyclohexyl)ethanoate (0.2 g, 0.588 mmol) and 5-(2,4-dichlorophenyl)-2-furancarbonyl chloride (0.16 g, 0.581 mmol) was added triethylamine (0.058 g, 0.574 mmol). The reaction was allowed to stir for ca. 15 hours and then reaction mixture partitioned between water and CH₂Cl₂. The CH₂Cl₂ layer was dried over magnesium sulfate, filtered and the solvent evaporated. Chromatography on silica gel with hexane/ethyl acetate gave 0.31 g of light yellow solid.

### Step 3

### (2S)-cyclohexyl({[3-({[5-(2,4-dichlorophenyl)-2-furanyl]carbonyl}amino)-2-naphthalenyl]carbonyl}amino)ethanoic acid

Lithium hydroxide monohydrate (0.13 g, 5.43 mmol) was added to a solution of methyl(2S)-cyclohexyl({[3-({[5-(2,4-dichlorophenyl)-2-furanyl]carbonyl}amino)-2-naphthalenyl]carbonyl}amino)ethanoate (0.31 g, 0.535 mmol) in dioxane:water/10:1 (7 ml). The mixture was stirred at RT overnight. The reaction mixture was acidified with 1N aqueous HCl and extracted with ethyl acetate. The organic phase was dried over sodium sulfate, filtered and concentrated in vacuo, triturated with hexanes/ethyl acetate to give 0.120 g (42% yield) of product. ES MS m/z 563 (M−H).

### Example 256

### (2S)-4-(methylsulfonyl)-1-{[3-({[(2,4,6-trimethylphenyl)amino]carbonyl}amino)-2-naphthalenyl]carbonyl}-2-piperazinecarboxylic acid

### Step 1

### Methyl(2S)-1-{[3-({[(2,4,6-trimethylphenyl)amino]carbonyl}amino)-2-naphthalenyl]carbonyl}-2-piperazinecarboxylate

1-(1,1-dimethylethyl) 3-methyl(3S)-4-{[3-({[(2,4,6-trimethylphenyl)amino]carbonyl}amino)-2-naphthalenyl]carbonyl}-1,3-piperazinedicarboxylate (0.5 g, 0.870 mmol) in 15 mL of CH₂Cl₂:TFA (2:1). The mixture as stirred at RT for ca. 15 h and the solvents were removed under reduced pressure to give the 0.8 g of the product.

### Step 2

### Methyl(2S)-4-(methylsulfonyl)-1-{[3-({[(2,4,6-trimethylphenyl)amino]carbonyl}amino)-2-naphthalenyl]carbonyl}-2-piperazinecarboxylate

A solution of methyl(2S)-1-{[3-({[(2,4,6-trimethylphenyl)amino]carbonyl}amino)-2-naphthalenyl]carbonyl}-2-piperazinecarboxylate (0.1 g, 0.211 mmol) in 3 ml of CH₂Cl₂ was cooled in an ice bath and to which was added triethylamine (0.032 g, 0.316 mmol) and methanesulfonyl chloride (0.027 g, 0.233 mmol). The reaction was allowed to come to room temperature stirring for 15 hours. The reaction was quenched with saturated sodium bicarbonate and the organics were dried over sodium sulfate, filtered and loaded directly onto silica for chromatography. Chromatography on silica gel with 5% MeOH in CH₂Cl₂ gave 0.2 g of product which still contained impurities. Material was further purified on Agilent semi-prep HPLC to give 33 mg of light yellow oil.

### Step 3

### (2S)-4-(methylsulfonyl)-1-{[3-({[(2,4,6-trimethylphenyl)amino]carbonyl}amino)-2-naphthalenyl]carbonyl}-2-piperazinecarboxylic acid

Lithium hydroxide monohydrate (0.014 g, 0.585 mmol) was added to a solution of methyl(2S)-4-(methylsulfonyl)-1-{[3-({[(2,4,6-trimethylphenyl)amino]carbonyl}amino)-2-naphthalenyl]carbonyl}-2-piperazinecarboxylate (0.033 g, 0.060 mmol) in dioxane:water/10:1 (3 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.012 g (19% yield) of product. ES MS m/z 539 (M+H).

### Example 257

### (2S)-1-{[3-({[(2,4,6-trimethylphenyl)amino]carbonyl}amino)-2-naphthalenyl]carbonyl}-2-piperidinecarboxylic acid

### Step 1

### Methyl(2S)-1-[(3-amino-2-naphthalenyl)carbonyl]-2-piperidinecarboxylate

HATU (1.14 g, 3.00 mmol) was added to a solution of 3-amino-2-naphthalenecarboxylic acid (0.5 g, 2.67 mmol), methyl(2S)-2-piperidinecarboxylate hydrochloride (0.58 g, 3.23 mmol) and diisopropylethylamine (0.38 g, 2.95 mmol) in 15 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.49 g of product as yellow oil.

### Step 2

### Methyl(2S)-1-{[3-({[(2,4,6-trimethylphenyl)amino]carbonyl}amino)-2-naphthalenyl]carbonyl}-2-piperidinecarboxylate

Methyl(2S)-1-[(3-amino-2-naphthalenyl)carbonyl]-2-piperidinecarboxylate (0.49 g, 1.57 mmol) in 10 mL of pyridine was treated with 2-isocyanato-1,3,5-trimethylbenzene (1.27 g, 7.86 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.18 g of product as a fluffy yellow foam.

### Step 3

### (2S)-1-{[3-({[(2,4,6-trimethylphenyl)amino]carbonyl}amino)-2-naphthalenyl]carbonyl}-2-piperidinecarboxylic acid

Lithium hydroxide monohydrate (0.091 g, 3.80 mmol) was added to a solution of methyl(2S)-1-{[3-({[(2,4,6-trimethylphenyl)amino]carbonyl}amino)-2-naphthalenyl]carbonyl}-2-piperidinecarboxylate (0.18 g, 0.380 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.23 g (100% yield) of product. ES MS m/z 460 (M+H).

### Example 258

### O-(1,1-dimethylethyl)-N-{[3-({[(2,4,6-trimethylphenyl)amino]carbonyl}amino)-2-naphthalenyl]carbonyl}-L-serine

### Step 1

### Methyl N-[(3-amino-2-naphthalenyl)carbonyl]-O-(1,1-dimethylethyl)-L-serinate

HATU (1.14 g, 3.00 mmol) was added to a solution of 3-amino-2-naphthalenecarboxylic acid (0.5 g, 2.67 mmol), methyl O-(1,1-dimethylethyl)-L-serinate hydrochloride (0.68 g, 3.22 mmol) and diisopropylethylamine (0.38 g, 2.95 mmol) in 15 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 1.16 g of product as yellow solid.

### Step 2

### Methyl O-(1,1-dimethylethyl)-N-{[3-({[(2,4,6-trimethylphenyl)amino]carbonyl}amino)-2-naphthalenyl]carbonyl}-L-serinate

Methyl N-[(3-amino-2-naphthalenyl)carbonyl]-O-(1,1-dimethylethyl)-L-serinate (1.16 g, 3.37 mmol) in 15 mL of pyridine was treated with 2-isocyanato-1,3,5-trimethylbenzene (2.72 g, 16.83 mmol) for ca. 15 h at RT. The reaction was quenched with 1N HCl and extracted with ethyl acetate. The organic layer dried over magnesium sulfate, filtered, and the solvent evaporated to give 1.01 g of product as amber oil.

### Step 3

### O-(1,1-dimethylethyl)-N-{[3-({[(2,4,6-trimethylphenyl)amino]carbonyl}amino)-2-naphthalenyl]carbonyl}-L-serine

Lithium hydroxide monohydrate (0.062 g, 2.59 mmol) was added to a solution of methyl O-(1,1-dimethylethyl)-N-{[3-({[(2,4,6-trimethylphenyl)amino]carbonyl}amino)-2-naphthalenyl]carbonyl}-L-serinate (0.130 g, 0.257 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.037 g (25% yield) of product. ES MS m/z 492 (M+H).

### Example 259

### 5-methyl-N-{[3-({[(2,4,6-trimethylphenyl)amino]carbonyl}amino)-2-naphthalenyl]carbonyl}norleucine

### Step 1

### Methyl(2E)-5-methyl-2-({[(phenylmethyl)oxy]carbonyl}amino)-2-hexenoate

To a solution of methyl[bis(methyloxy)phosphoryl]({[(phenylmethyl)oxy]carbonyl}amino)acetate (2.12 g, 6.40 mmol) in CH₂Cl₂ was added DBU (0.92 g, 6.02 mmol) and the resulting solution was stirred at RT for 10 minutes. To that was then added 3-methylbutanal (0.5 g, 5.81 mmol), and the reaction was stirred for 16 hours at RT. The reaction was quenched with 1N HCl and the CH₂Cl₂ layer dried over sodium sulfate, filtered, and the solvent evaporated. Chromatography on silica gel with hexane/ethyl acetate gave 1.34 g of product as clear oil.

### Step 2

### Methyl 5-methylnorleucinate

Palladium (10% weight on activated carbon, catalytic amount) was added to a solution of methyl(2E)-5-methyl-2-({[(phenylmethyl)oxy]carbonyl}amino)-2-hexenoate (1.34 g, 4.60 mmol) in 25 ml of EtOH in a flask under nitrogen. A balloon of H₂ was then affixed to the reaction flask and the reaction was stirred for 16 hours at RT. The reaction was then filtered through a pad of Celite and the solvent evaporated to give 0.46 g of yellow oil.

### Step 3

### Methyl N-[(3-amino-2-naphthalenyl)carbonyl]-5-methylnorleucinate

HATU (1.10 g, 2.89 mmol) was added to a solution of 3-amino-2-naphthalenecarboxylic acid (0.45 g, 2.40 mmol), methyl 5-methylnorleucinate (0.46 g, 2.89 mmol) and diisopropylethylamine (0.29 g, 2.24 mmol) in 20 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.44 g of product as yellow oil.

### Step 4

### Methyl 5-methyl-N-{[3-({[(2,4,6-trimethylphenyl)amino]carbonyl}amino)-2-naphthalenyl]carbonyl}norleucinate

Methyl N-[(3-amino-2-naphthalenyl)carbonyl]-5-methylnorleucinate (0.44 g, 1.34 mmol) in 20 mL of pyridine was treated with 2-isocyanato-1,3,5-trimethylbenzene (1.08 g, 6.68 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.34 g of product as a light tan semi-solid.

### Step 5

### 5-methyl-N-{[3-({[(2,4,6-trimethylphenyl)amino]carbonyl}amino)-2-naphthalenyl]carbonyl}norleucine

Lithium hydroxide monohydrate (0.17 g, 7.10 mmol) was added to a solution of methyl 5-methyl-N-{[3-({[(2,4,6-trimethylphenyl)amino]carbonyl}amino)-2-naphthalenyl]carbonyl}norleucinate (0.34 g, 0.694 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.34 g (100% yield) of product. ES MS m/z 476 (M+H).

### Example 260

### 6,6,6-trifluoro-N-{[3-({[(2,4,6-trimethylphenyl)amino]carbonyl}amino)-2-naphthalenyl]carbonyl}norleucine

### Step 1

### Methyl(2E)-6,6,6-trifluoro-2-({[(phenylmethyl)oxy]carbonyl}amino)-2-hexenoate

To a solution of methyl[bis(methyloxy)phosphoryl]({[(phenylmethyl)oxy]carbonyl}amino)acetate (1.44 g, 4.35 mmol) in CH₂Cl₂ was added DBU (0.64 g, 4.21 mmol) and the resulting solution was stirred at RT for 10 minutes. To that was then added 4,4,4-trifluorobutanal (0.5 g, 3.97 mmol), and the reaction was stirred for 16 hours at RT. The reaction was quenched with 1N HCl and the CH₂Cl₂ layer dried over sodium sulfate, filtered, and the solvent evaporated. Chromatography on silica gel with hexane/ethyl acetate gave 0.39 g of product as a white solid.

### Step 2

### Methyl 6,6,6-trifluoronorleucinate

Palladium (10% weight on activated carbon, catalytic amount) was added to a solution of methyl(2E)-6,6,6-trifluoro-2-({[(phenylmethyl)oxy]carbonyl}amino)-2-hexenoate (0.39 g, 1.18 mmol) in 25 ml of EtOH in a flask under nitrogen. A balloon of H₂ was then affixed to the reaction flask and the reaction was stirred for 16 hours at RT. The reaction was then filtered through a pad of Celite and the solvent evaporated to give 0.16 g of white semi-solid.

### Step 3

### Methyl N-[(3-amino-2-naphthalenyl)carbonyl]-6,6,6-trifluoronorleucinate

HATU (0.30 g, 0.789 mmol) was added to a solution of 3-amino-2-naphthalenecarboxylic acid (0.125 g, 0.668 mmol), methyl 6,6,6-trifluoronorleucinate (0.16 g, 0.803 mmol) and diisopropylethylamine (0.104 g, 0.803 mmol) in 12 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.13 g of product as orange oil.

### Step 4

### Methyl 6,6,6-trifluoro-N-{[3-({[(2,4,6-trimethylphenyl)amino]carbonyl}amino)-2-naphthalenyl]carbonyl}norleucinate

Methyl N-[(3-amino-2-naphthalenyl)carbonyl]-6,6,6-trifluoronorleucinate (0.13 g, 0.353 mmol) in 20 mL of pyridine was treated with 2-isocyanato-1,3,5-trimethylbenzene (0.29 g, 1.79 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.17 g of product as a light yellow solid.

### Step 5

### 6,6,6-trifluoro-N-{[3-({[(2,4,6-trimethylphenyl)amino]carbonyl}amino)-2-naphthalenyl]carbonyl}norleucine

Lithium hydroxide monohydrate (0.038 g, 1.59 mmol) was added to a solution of methyl 6,6,6-trifluoro-N-{[3-({[(2,4,6-trimethylphenyl)amino]carbonyl}amino)-2-naphthalenyl]carbonyl}norleucinate (0.17 g, 0.321 mmol) in dioxane:water/10:1 (5 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.109 g (66% yield) of product. ES MS m/z 516 (M+H).

### Example 260

### O-(1,1-dimethylethyl)-N-{[3-({[(2,4,6-trimethylphenyl)amino]carbonyl}amino)-2-naphthalenyl]carbonyl}-L-threonine

### Step 1

### Methyl N-[(3-amino-2-naphthalenyl)carbonyl]-O-(1,1-dimethylethyl)-L-threoninate

HATU (1.22 g, 3.21 mmol) was added to a solution of 3-amino-2-naphthalenecarboxylic acid (0.5 g, 2.67 mmol), methyl O-(1,1-dimethylethyl)-L-threoninate hydrochloride (0.72 g, 3.19 mmol) and diisopropylethylamine (0.41 g, 3.21 mmol) in 20 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.72 g of product as amber oil.

### Step 2

### Methyl O-(1,1-dimethylethyl)-N-{[3-({[(2,4,6-trimethylphenyl)amino]carbonyl}amino)-2-naphthalenyl]carbonyl}-L-threoninate

Methyl N-[(3-amino-2-naphthalenyl)carbonyl]-O-(1,1-dimethylethyl)-L-threoninate (0.72 g, 2.01 mmol) in 12 mL of pyridine was treated with 2-isocyanato-1,3,5-trimethylbenzene (1.62 g, 10.02 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.85 g of product as a yellow fluffy tar.

### Step 3

### O-(1,1-dimethylethyl)-N-{[3-({[(2,4,6-trimethylphenyl)amino]carbonyl}amino)-2-naphthalenyl]carbonyl}-L-threonine

Lithium hydroxide monohydrate (0.39 g, 16.28 mmol) was added to a solution of methyl O-(1,1-dimethylethyl)-N-{[3-({[(2,4,6-trimethylphenyl)amino]carbonyl}amino)-2-naphthalenyl]carbonyl}-L-threoninate (0.85 g, 1.64 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.523 g (65% yield) of product. ES MS m/z 506 (M+H).

### Example 262

### 2-({[3-({[(2,4,6-trimethylphenyl)amino]carbonyl}amino)-2-naphthalenyl]carbonyl}amino)heptanoic acid

### Step 1

### Methyl(2E)-2-({[(phenylmethyl)oxy]carbonyl}amino)-2-heptenoate

To a solution of methyl[bis(methyloxy)phosphoryl]({[(phenylmethyl)oxy]-carbonyl}amino)acetate (2.12 g, 6.40 mmol) in CH₂Cl₂ was added DBU (0.93 g, 6.08 mmol) and the resulting solution was stirred at RT for 10 minutes. To that was then added pentanal (0.5 g, 5.81 mmol), and the reaction was stirred for 16 hours at RT. The reaction was quenched with 1N HCl and the CH₂Cl₂ layer dried over sodium sulfate, filtered, and the solvent evaporated. Chromatography on silica gel with hexane/ethyl acetate gave 0.92 g of product as a colorless oil.

### Step 2

### Methyl 2-aminoheptanoate

Palladium (10% weight on activated carbon, catalytic amount) was added to a solution of methyl(2E)-2-({[(phenylmethyl)oxy]carbonyl}amino)-2-heptenoate (0.92 g, 3.16 mmol) in 30 ml of EtOH in a flask under nitrogen. A balloon of H₂ was then affixed to the reaction flask and the reaction was stirred for 16 hours at RT. The reaction was then filtered through a pad of Celite and the solvent evaporated to give 0.32 g of light yellow oil.

### Step 3

### Methyl 2-{[(3-amino-2-naphthalenyl)carbonyl]amino}heptanoate

HATU (0.76 g, 2.00 mmol) was added to a solution of 3-amino-2-naphthalenecarboxylic acid (0.31 g, 1.66 mmol), methyl 2-aminoheptanoate (0.32 g, 2.01 mmol) and diisopropylethylamine (0.26 g, 2.01 mmol) in 20 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.21 g of product as a light amber oil.

### Step 4

### Methyl 2-({[3-({[(2,4,6-trimethylphenyl)amino]carbonyl}amino)-2-naphthalenyl]carbonyl}amino)heptanoate

Methyl 2-{[(3-amino-2-naphthalenyl)carbonyl]amino}heptanoate (0.21 g, 0.639 mmol) in 20 mL of pyridine was treated with 2-isocyanato-1,3,5-trimethylbenzene (0.52 g, 3.22 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.220 g of product as a light orange solid.

### Step 5

### 2-({[3-({[(2,4,6-trimethylphenyl)amino]carbonyl}amino)-2-naphthalenyl]carbonyl}amino)heptanoic acid

Lithium hydroxide monohydrate (0.11 g, 4.59 mmol) was added to a solution of methyl 2-({[3-({[(2,4,6-trimethylphenyl)amino]carbonyl}amino)-2-naphthalenyl]carbonyl}amino)heptanoate (0.22 g, 0.449 mmol) in dioxane:water/10:1 (20 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.277 g (100% yield) of product. ES MS m/z 476 (M+H).

### Example 263

### 3-({[(2,4,6-trimethylphenyl)amino]carbonyl}amino)-2-naphthalenecarboxylic acid

3-amino-2-naphthalenecarboxylic acid (5.00 g, 26.71 mmol) in 100 mL of DMF was treated with triethylamine (5.40 g, 53.37 mmol) and 2-isocyanato-1,3,5-trimethylbenzene (4.7 g, 29.16 mmol) and was heated to 70° C. for ca. 3 hours. The reaction was quenched with 1N HCl and extracted with ethyl acetate. The organic layer was dried over magnesium sulfate, filtered, and the solvent evaporated to give 7.95 g (84%) of product. ES MS m/z 349 (M+H).

### Example 264

### 5,7,7-trimethyl-2-({[3-({[(2,4,6-trimethylphenyl)amino]carbonyl}amino)-2-naphthalenyl]carbonyl}amino)octanoic acid

### Step 1

### Methyl(2E)-5,7,7-trimethyl-2-({[(phenylmethyl)oxy]carbonyl}amino)-2-octenoate

To a solution of methyl[bis(methyloxy)phosphoryl]({[(phenylmethyl)oxy]carbonyl}amino)acetate (1.28 g, 3.86 mmol) in CH₂Cl₂ was added DBU (0.57 g, 3.74 mmol) and the resulting solution was stirred at RT for 10 minutes. To that was then added 3,5,5-trimethylhexanal (0.5 g, 3.52 mmol), and the reaction was stirred for 16 hours at RT. The reaction was quenched with 1N HCl and the CH₂Cl₂ layer dried over sodium sulfate, filtered, and the solvent evaporated. Chromatography on silica gel with hexane/ethyl acetate gave 1.36 g of product as a colorless oil.

### Step 2

### methyl 2-amino-5,7,7-trimethyloctanoate

Palladium (10% weight on activated carbon, catalytic amount) was added to a solution of methyl(2E)-5,7,7-trimethyl-2-({[(phenylmethyl)oxy]carbonyl}amino)-2-octenoate (1.36 g, 3.91 mmol) in 25 ml of EtOH in a flask under nitrogen. A balloon of H₂ was then affixed to the reaction flask and the reaction was stirred for 16 hours at RT. The reaction was then filtered through a pad of Celite and the solvent evaporated to give 0.85 g of light yellow oil.

### Step 3

### Methyl 2-{[(3-amino-2-naphthalenyl)carbonyl]amino}-5,7,7-trimethyloctanoate

HATU (1.48 g, 3.89 mmol) was added to a solution of 3-amino-2-naphthalenecarboxylic acid (0.62 g, 3.31 mmol), methyl 2-amino-5,7,7-trimethyloctanoate (0.85 g, 3.95 mmol) and diisopropylethylamine (0.50 g, 3.90 mmol) in 20 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 1.04 g of product as amber oil.

### Step 4

### Methyl 5,7,7-trimethyl-2-({[3-({[(2,4,6-trimethylphenyl)amino]carbonyl}amino)-2-naphthalenyl]carbonyl}amino)octanoate

Methyl 2-{[(3-amino-2-naphthalenyl)carbonyl]amino}-5,7,7-trimethyloctanoate (1.04 g, 2.70 mmol) in 20 mL of pyridine was treated with 2-isocyanato-1,3,5-trimethylbenzene (2.19 g, 13.55 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.91 g of product as amber oil.

### Step 5

### 5,7,7-trimethyl-2-({[3-({[(2,4,6-trimethylphenyl)amino]carbonyl}amino)-2-naphthalenyl]carbonyl}amino)octanoic acid

Lithium hydroxide monohydrate (0.20 g, 8.35 mmol) was added to a solution of methyl 5,7,7-trimethyl-2-({[3-({[(2,4,6-trimethylphenyl)amino]carbonyl}amino)-2-naphthalenyl]carbonyl}amino)octanoate (0.91 g, 1.67 mmol) in dioxane:water/10:1 (20 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.87 g (97% yield) of product. ES MS m/z 532 (M+H).

### Example 265

### N-{[3-({[(2,4,6-trimethylphenyl)amino]carbonyl}amino)-2-naphthalenyl]carbonyl}-L-leucine

### Step 1

### Methyl N-[(3-amino-2-naphthalenyl)carbonyl]-L-leucinate

HATU (1.14 g, 3.00 mmol) was added to a solution of 3-amino-2-naphthalenecarboxylic acid (0.5 g, 2.67 mmol), methyl L-leucinate hydrochloride (0.58 g, 3.19 mmol) and diisopropylethylamine (0.38 g, 2.98 mmol) in 20 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.76 g of product as yellow oil.

### Step 2

### Methyl N-{[3-({[(2,4,6-trimethylphenyl)amino]carbonyl}amino)-2-naphthalenyl]carbonyl}-L-leucinate

Methyl N-[(3-amino-2-naphthalenyl)carbonyl]-L-leucinate (0.76 g, 2.42 mmol) in 20 mL of pyridine was treated with 2-isocyanato-1,3,5-trimethylbenzene (1.96 g, 12.13 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.75 g of product as amber semi-solid.

### Step 3

### N-{[3-({[(2,4,6-trimethylphenyl)amino]carbonyl}amino)-2-naphthalenyl]carbonyl}-L-leucine

Lithium hydroxide monohydrate (0.38 g, 15.87 mmol) was added to a solution of methyl N-{[3-({[(2,4,6-trimethylphenyl)amino]carbonyl}amino)-2-naphthalenyl]carbonyl}-L-leucinate (0.75 g, 1.58 mmol) in dioxane:water/10:1 (20 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.35 g (47% yield) of product. ES MS m/z 462 (M+H).

### Example 266

### N-{[3-({[(2,4,6-trimethylphenyl)amino]carbonyl}amino)-2-naphthalenyl]carbonyl}-L-isoleucine

### Step 1

### Methyl N-[(3-amino-2-naphthalenyl)carbonyl]-L-isoleucinate

HATU (1.14 g, 3.00 mmol) was added to a solution of 3-amino-2-naphthalenecarboxylic acid (0.5 g, 2.67 mmol), methyl L-alloisoleucinate hydrochloride (0.58 g, 3.19 mmol) and diisopropylethylamine (0.38 g, 2.98 mmol) in 20 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.92 g of product as yellow oil.

### Step 2

### Methyl N-{[3-({[(2,4,6-trimethylphenyl)amino]carbonyl}amino)-2-naphthalenyl]carbonyl}-L-isoleucinate

Methyl N-[(3-amino-2-naphthalenyl)carbonyl]-L-isoleucinate (0.92 g, 2.93 mmol) in 20 mL of pyridine was treated with 2-isocyanato-1,3,5-trimethylbenzene (2.37 g, 14.67 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.63 g of product as amber semi-solid.

### Step 3

### N-{[3-({[(2,4,6-trimethylphenyl)amino]carbonyl}amino)-2-naphthalenyl]carbonyl}-L-isoleucine

Lithium hydroxide monohydrate (0.32 g, 13.36 mmol) was added to a solution of methyl N-{[3-({[(2,4,6-trimethylphenyl)amino]carbonyl}amino)-2-naphthalenyl]carbonyl}-L-isoleucinate (0.63 g, 1.32 mmol) in dioxane:water/10:1 (20 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.314 g (52% yield) of product as a light yellow fluffy solid. ES MS m/z 462 (M+H).

### Example 267

### N-{[3-({[(2,4,6-trimethylphenyl)amino]carbonyl}amino)-2-naphthalenyl]carbonyl}-L-norvaline

### Step 1

### Methyl N-[(3-amino-2-naphthalenyl)carbonyl]-L-norvalinate

HATU (1.14 g, 3.00 mmol) was added to a solution of 3-amino-2-naphthalenecarboxylic acid (0.5 g, 2.67 mmol), methyl L-norvalinate hydrochloride (0.5 g, 2.98 mmol) and diisopropylethylamine (0.38 g, 2.98 mmol) in 20 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.72 g of product as yellow oil.

### Step 2

### Methyl N-{[3-({[(2,4,6-trimethylphenyl)amino]carbonyl}amino)-2-naphthalenyl]carbonyl}-L-norvalinate

Methyl N-[(3-amino-2-naphthalenyl)carbonyl]-L-norvalinate (0.72 g, 2.4 mmol) in 20 mL of pyridine was treated with 2-isocyanato-1,3,5-trimethylbenzene (1.94 g, 12.00 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.8 g of product as light yellow semi-solid.

### Step 3

### N-{[3-({[(2,4,6-trimethylphenyl)amino]carbonyl}amino)-2-naphthalenyl]carbonyl}-L-norvaline

Lithium hydroxide monohydrate (0.41 g, 17.12 mmol) was added to a solution of methyl N-{[3-({[(2,4,6-trimethylphenyl)amino]carbonyl}amino)-2-naphthalenyl]carbonyl}-L-norvalinate (0.8 g, 1.73 mmol) in dioxane:water/10:1 (25 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.77 g (100% yield) of product as a tan fluffy solid. ES MS m/z 448 (M+H).

### Example 268

### O-(1,1-dimethylethyl)-N-[(3-{[(2,4,6-trimethylphenyl)acetyl]amino}-2-naphthalenyl)carbonyl]-L-threonine

### Step 1

### Methyl N-[(3-amino-2-naphthalenyl)carbonyl]-O-(1,1-dimethylethyl)-L-threoninate

HATU (2.44 g, 6.42 mmol) was added to a solution of 3-amino-2-naphthalenecarboxylic acid (1.0 g, 5.34 mmol), methyl O-(1,1-dimethylethyl)-L-threoninate hydrochloride (1.44 g, 6.38 mmol) and diisopropylethylamine (0.83 g, 6.42 mmol) in 40 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 1.3 g of product as amber oil.

### Step 2

### Methyl O-(1,1-dimethylethyl)-N-[(3-{[(2,4,6-trimethylphenyl)acetyl]amino}-2-naphthalenyl)carbonyl]-L-threoninate

HATU (0.68 g, 1.79 mmol) was added to a solution of methyl N-[(3-amino-2-naphthalenyl)carbonyl]-O-(1,1-dimethylethyl)-L-threoninate (0.32 g, 0.89 mmol), (2,4,6-trimethylphenyl)acetic acid (0.19 g, 1.07 mmol) and diisopropylethylamine (0.14 g, 1.09 mmol) in 20 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.41 g of product as yellow oil.

### Step 3

### O-(1,1-dimethylethyl)-N-[(3-{[(2,4,6-trimethylphenyl)acetyl]amino}-2-naphthalenyl)carbonyl]-L-threonine

Lithium hydroxide monohydrate (0.19 g, 7.93 mmol) was added to a solution of methyl O-(1,1-dimethylethyl)-N-[(3-{[(2,4,6-trimethylphenyl)acetyl]amino}-2-naphthalenyl)carbonyl]-L-threoninate (0.41 g, 0.79 mmol) in dioxane:water/10:1 (20 ml). The mixture was stirred at RT overnight. The reaction mixture was acidified with 1N aqueous HCl and extracted with ethyl acetate. The organic phase was dried over magnesium sulfate, filtered and concentrated in vacuo. Chromatography on silica gel with CH₂Cl₂/MeOH gave 0.0705 g (18% yield) of product as a cream fluffy solid. ES MS m/z 505 (M+H).

### Example 269

### 2-methyl-N-{[3-({[(2,4,6-trimethylphenyl)amino]carbonyl}amino)-2-naphthalenyl]carbonyl}leucine

### Step 1

### N-{[(1,1-dimethylethyl)oxy]carbonyl}-2-methylleucine

To a solution of 2-methylleucine (0.5 g, 3.44 mmol) and triethylamine (1.05 g, 10.33 mmol) in CH₂Cl₂ (10 ml) cooled to 0° C. was added di-tert-butyl dicarbonate (1.65 g, 7.59 mmol) portionwise, followed by DMAP (0.51 g, 4.18 mmol). The reaction was allowed to warm to RT and stir 16 hours. The reaction was then quenched with 0.1N HCl and extracted with CH₂Cl₂. The CH₂Cl₂ layer was dried over sodium sulfate, filtered and stripped to give 0.825 g of product as a light yellow solid.

### Step 2

### Methyl N-{[(1,1-dimethylethyl)oxy]carbonyl}-2-methylleucinate

To a solution of N-{[(1,1-dimethylethyl)oxy]carbonyl}-2-methylleucine (0.8 g, 3.26 mmol) in MeOH (25 ml) was added TMS-diazomethane (0.74 g, 6.5 mmol), and the rection was allowed to stir for 16 hours at RT. The organic phase was concentrated in vacuo to give 1.17 g of product as a dark brown semi-solid.

### Step 3

### Methyl 2-methylleucinate

Methyl N-{[(1,1-dimethylethyl)oxy]carbonyl}-2-methylleucinate was dissolved in 2:1 CH2Cl2:TFA (30 ml) and allowed to stir at RT for 1 hour. The organic phase was concentrated in vacuo and then dissolved in EtOAc and washed with 1N NaOH, dried over magnesium sulfate, filtered and concentrated in vacuo to give 0.43 g of product as a brown solid.

### Step 4

### Methyl N-[(3-amino-2-naphthalenyl)carbonyl]-2-methylleucinate

HATU (0.48 g, 1.26 mmol) was added to a solution of 3-amino-2-naphthalenecarboxylic acid (0.2 g, 1.07 mmol), methyl 2-methylleucinate (0.2 g, 1.26 mmol) and diisopropylethylamine (0.16 g, 1.26 mmol) in 20 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.078 g of product as amber oil.

### Step 5

### Methyl 2-methyl-N-{[3-({[(2,4,6-trimethylphenyl)amino]carbonyl}amino)-2-naphthalenyl]carbonyl}leucinate

Methyl N-[(3-amino-2-naphthalenyl)carbonyl]-2-methylleucinate (0.078 g, 0.238 mmol) in 10 mL of pyridine was treated with 2-isocyanato-1,3,5-trimethylbenzene (0.19 g, 1.18 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.066 g of product as yellow oil.

### Step 6

### 2-methyl-N-{[3-({[(2,4,6-trimethylphenyl)amino]carbonyl}amino)-2-naphthalenyl]carbonyl}leucine

Lithium hydroxide monohydrate (0.032 g, 1.34 mmol) was added to a solution of methyl 2-methyl-N-{[3-({[(2,4,6-trimethylphenyl)amino]carbonyl}amino)-2-naphthalenyl]carbonyl}leucinate (0.066 g, 0.135 mmol) in dioxane:water/10:1 (1 0 ml). The mixture was stirred at RT overnight. The reaction mixture was acidified with 1N aqueous HCl and extracted with ethyl acetate. The organic phase was dried over magnesium sulfate, filtered and concentrated in vacuo. Trituration with hexanes gave 0.033 g (53% yield) of product as a light yellow solid. ES MS m/z 476 (M+H).

### Example 270

### (2S)-cyclohexyl({[3-({[5-(2,4,6-trimethylphenyl)-2-furanyl]carbonyl}amino)-2-naphthalenyl]carbonyl}amino)ethanoic acid

### Step 1

### Methyl(2S)-{[(3-{[(5-bromo-2-furanyl)carbonyl]amino}-2-naphthalenyl)carbonyl]amino}(cyclohexyl)ethanoate

Triethylamine (0.17 g, 1.72 mmol) was added to a solution of methyl(2S)-{[(3-amino-2-naphthalenyl)carbonyl]amino}(cyclohexyl)ethanoate (0.57 g, 1.67 mmol) and 5-bromo-2-furancarbonyl chloride (0.35 g, 1.67 mmol) in CH₂Cl₂ (20 ml) and allowed to stir at RT for 16 hours. The reaction was then partiotioned between 1N HCl and EtOAc and the EtOAc layer dried over magnesium sulfate, filtered and concentrated in vacuo to give 0.68 g of fluffy orange glassy solid.

### Step 2

### Methyl(2S)-cyclohexyl({[3-({[5-(2,4,6-trimethylphenyl)-2-furanyl]carbonyl}amino)-2-naphthalenyl]carbonyl}amino)ethanoate

To a solution of methyl(2S)-{[(3-{[(5-bromo-2-furanyl)carbonyl]amino}-2-naphthalenyl)carbonyl]amino}(cyclohexyl)ethanoate (0.11 g, 0.214 mmol) in DME (3 ml) was added tetrakis(triphenylmethyl)palladium (0.007 g, 0.006 mmol), (2,4,6-trimethylphenyl)boronic acid (0.053 g, 0.323 mmol) and 2M Na2CO3 (0.2 ml). The reaction was heated to 110oC for 16 hours and then loaded directly onto silica. Chromatography on silica gel with hexane/ethyl acetate gave 0.07 g of product as yellow oil.

### Step 3

### (2S)-cyclohexyl({[3-({[5-(2,4,6-trimethylphenyl)-2-furanyl]carbonyl}amino)-2-naphthalenyl]carbonyl}amino)ethanoic acid

Lithium hydroxide monohydrate (0.030 g, 1.25 mmol) was added to a solution of methyl(2S)-cyclohexyl({[3-({[5-(2,4,6-trimethylphenyl)-2-furanyl]carbonyl}amino)-2-naphthalenyl]carbonyl}amino)ethanoate (0.07 g, 0.127 mmol) in dioxane:water/10:1 (5 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.033 g (48% yield) of product as a light yellow solid. ES MS m/z 539 (M+H).

### Example 271

### O-butyl-N-{[3-({[(2,4,6-trimethylphenyl)amino]carbonyl}amino)-2-naphthalenyl]carbonyl}-L-serine

### Step 1

### 2-methyl 1-(phenylmethyl) (2S)-1,2-aziridinedicarboxylate

To a solution of methyl(2S)-1-(triphenylmethyl)-2-aziridinecarboxylate (5.0 g, 14.56 mmol) in CHCl₃ (50 ml) and MeOH (12 ml) cooled to −10° C. was added 24 ml of TFA and allowed to stir at −10° C. for 2 hours. The reaction was then concentrated in vacuo and taken up in another 50 ml of CHCl₃. The resulting solution was then cooled to 0° C. and triethylamine (3.69 g, 36.51 mmol) was added followed by benzyl chloroformate (2.49 g, 14.6 mmol). After stirring for 2 hours at 0° C., the reaction was diluted with H₂O and the pH was adjusted to 8 with saturated NaHCO₃. The organic layer was dried over magnesium sulfate, filtered and loaded onto silica for purification. Chromatography on silica gel with hexane/ethyl acetate gave 2.7 g of product as a clear oil.

### Step 2

### Methyl O-butyl-N-{[(phenylmethyl)oxy]carbonyl}-L-serinate

To a solution of 2-methyl 1-(phenylmethyl) (2S)-1,2-aziridinedicarboxylate (0.5 g, 2.26 mmol) in CHCl₃ (5 ml) was added 1-butanol (6.49 g, 87.53 mmol) and boron trifluoride diethyl etherate (5 drops) and stirred for 16 hours. The reaction was quenched with H₂O and extracted with CH₂Cl2. The CH2Cl2 layer was dried over magnesium sulfate, filtered and concentrated in vacuo to give 0.6 g of product as a clear oil.

### Step 3

### Methyl O-butyl-L-serinate

Palladium (10% weight on activated carbon, catalytic amount) was added to a solution of methyl O-butyl-N-{[(phenylmethyl)oxy]carbonyl}-L-serinate (0.6 g, 1.94 mmol) in 12 ml of EtOH in a flask under nitrogen. A balloon of H₂ was then affixed to the reaction flask and the reaction was stirred for 2 hours at RT. The reaction was then filtered through a filter paper and the solvent evaporated to give 0.26 g of clear oil.

### Step 4

### Methyl N-[(3-amino-2-naphthalenyl)carbonyl]-O-butyl-L-serinate

HATU (0.61 g, 1.60 mmol) was added to a solution of 3-amino-2-naphthalenecarboxylic acid (0.24 g, 1.28 mmol), methyl O-butyl-L-serinate (0.26 g, 1.61 mmol) and diisopropylethylamine (0.21 g, 1.61 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.2 g of amber oil.

### Step 5

### Methyl O-butyl-N-{[3-({[(2,4,6-trimethylphenyl)amino]carbonyl}amino)-2-naphthalenyl]carbonyl}-L-serinate

Methyl N-[(3-amino-2-naphthalenyl)carbonyl]-O-butyl-L-serinate (0.2 g, 0.581 mmol) in 8 mL of pyridine was treated with 2-isocyanato-1,3,5-trimethylbenzene (0.47 g, 2.91 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.3 g of product as a cream solid.

### Step 6

### O-butyl-N-{[3-({[(2,4,6-trimethylphenyl)amino]carbonyl}amino)-2-naphthalenyl]carbonyl}-L-serine

Lithium hydroxide monohydrate (0.14 g, 5.85 mmol) was added to a solution of methyl O-butyl-N-{[3-({[(2,4,6-trimethylphenyl)amino]carbonyl}amino)-2-naphthalenyl]carbonyl}-L-serinate (0.3 g, 0.593 mmol) in dioxane:water/10:1 (5 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.22 g (76% yield) of product as a cream solid. ES MS m/z 492 (M+H).

### Example 272

### O-[2-(methyloxy)ethyl]-N-{[3-({[(2,4,6-trimethylphenyl)amino]carbonyl}amino)-2-naphthalenyl]carbonyl}-L-serine

### Step 1

### Methyl O-[2-(methyloxy)ethyl]-N-{[(phenylmethyl)oxy]carbonyl}-L-serinate

To a solution of 2-methyl 1-(phenylmethyl) (2S)-1,2-aziridinedicarboxylate (0.5 g, 2.26 mmol) in CHCl₃ (5 ml) was added 2-(methyloxy)ethanol (7.72 g, 101.45 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 0.63 g of product as a clear oil.

### Step 2

### Methyl O-[2-(methyloxy)ethyl]-L-serinate

Palladium (10% weight on activated carbon, catalytic amount) was added to a solution of methyl O-[2-(methyloxy)ethyl]-N-{[(phenylmethyl)oxy]carbonyl}-L-serinate (0.63 g, 2.02 mmol) in 12 ml of EtOH in a flask under nitrogen. A balloon of H₂ was then affixed to the reaction flask and the reaction was stirred for 2 hours at RT. The reaction was then filtered through a filter paper and the solvent evaporated to give 0.27 g of clear oil.

### Step 3

### Methyl N-[(3-amino-2-naphthalenyl)carbonyl]-O-[2-(methyloxy)ethyl]-L-serinate

HATU (0.57 g, 1.49 mmol) was added to a solution of 3-amino-2-naphthalenecarboxylic acid (0.24 g, 1.28 mmol), methyl O-[2-(methyloxy)ethyl]-L-serinate (0.27 g, 1.52 mmol) and diisopropylethylamine (0.19 g, 1.49 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.31 g of amber oil.

### Step 4

### Methyl O-[2-(methyloxy)ethyl]-N-{[3-({[(2,4,6-trimethylphenyl)amino]carbonyl}amino)-2-naphthalenyl]carbonyl}-L-serinate

Methyl N-[(3-amino-2-naphthalenyl)carbonyl]-O-[2-(methyloxy)ethyl]-L-serinate (0.3 g, 0.87 mmol) in 8 mL of pyridine was treated with 2-isocyanato-1,3,5-trimethylbenzene (0.7 g, 4.33 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.18 g of product as a tan semi-solid.

### Step 5

### O-[2-(methyloxy)ethyl]-N-{[3-({[(2,4,6-trimethylphenyl)amino]carbonyl}amino)-2-naphthalenyl]carbonyl}-L-serine

Lithium hydroxide monohydrate (0.085 g, 3.55 mmol) was added to a solution of methyl O-[2-(methyloxy)ethyl]-N-{[3-({[(2,4,6-trimethylphenyl)amino]carbonyl}amino)-2-naphthalenyl]carbonyl}-L-serinate (0.18 g, 0.355 mmol) in dioxane:water/10:1 (8 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.098 g (58% yield) of product as a cream solid. ES MS m/z 494 (M+H).

### Example 273

### O-ethyl-N-{[3-({[(2,4,6-trimethylphenyl)amino]carbonyl}amino)-2-naphthalenyl]carbonyl}-L-serine

### Step 1

### 2-methyl 1-(phenylmethyl) (2S)-1,2-aziridinedicarboxylate

To a solution of methyl(2S)-1-(triphenylmethyl)-2-aziridinecarboxylate (5.0 g, 14.56 mmol) in CHCl₃ (50 ml) and MeOH (12 ml) cooled to −10° C. was added 24 ml of TFA and allowed to stir at −10° C. for 2 hours. The reaction was then concentrated in vacuo and taken up in another 50 ml of CHCl₃. The resulting solution was then cooled to 0° C. and triethylamine (3.69 g, 36.51 mmol) was added followed by benzyl chloroformate (2.49 g, 14.6 mmol). After stirring for 2 hours at 0° C., the reaction was diluted with H₂O and the pH was adjusted to 8 with saturated NaHCO₃. The organic layer was dried over magnesium sulfate, filtered and loaded onto silica for purification. Chromatography on silica gel with hexane/ethyl acetate gave 2.53 g of product as a clear oil.

### Step 2

### Methyl O-ethyl-N-{[(phenylmethyl)oxy]carbonyl}-L-serinate

To a solution of 2-methyl 1-(phenylmethyl) (2S)-1,2-aziridinedicarboxylate (0.5 g, 2.26 mmol) in CHCl₃ (5 ml) was added ethanol (6.32 g, 137.18 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 0.52 g of product as a clear oil.

### Step 3

### Methyl O-ethyl-L-serinate

Palladium (10% weight on activated carbon, catalytic amount) was added to a solution of methyl O-ethyl-N-{[(phenylmethyl)oxy]carbonyl}-L-serinate. (0.52 g, 1.85 mmol) in 10 ml of EtOH in a flask under nitrogen. A balloon of H₂ was then affixed to the reaction flask and the reaction was stirred for 2 hours at RT. The reaction was then filtered through a filter paper and the solvent evaporated to give 0.16 g of clear oil.

### Step 4

### Methyl N-[(3-amino-2-naphthalenyl)carbonyl]-O-ethyl-L-serinate

HATU (0.34 g, 0.89 mmol) was added to a solution of 3-amino-2-naphthalenecarboxylic acid (0.17 g, 0.91 mmol), methyl O-ethyl-L-serinate (0.16 g, 1.09 mmol) and diisopropylethylamine (0.12 g, 0.92 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.18 g of amber oil.

### Step 5

### Methyl O-ethyl-N-{[3-({[(2,4,6-trimethylphenyl)amino]carbonyl}amino)-2-naphthalenyl]carbonyl}-L-serinate

Methyl N-[(3-amino-2-naphthalenyl)carbonyl]-O-ethyl-L-serinate (0.18 g, 0.57 mmol) in 10 mL of pyridine was treated with 2-isocyanato-1,3,5-trimethylbenzene (0.46 g, 2.85 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.16 g of product as a white solid.

### Step 6

### O-ethyl-N-{[3-({[(2,4,6-trimethylphenyl)amino]carbonyl}amino)-2-naphthalenyl]carbonyl}-L-serine

Lithium hydroxide monohydrate (0.080 g, 3.34 mmol) was added to a solution of methyl O-ethyl-N-{[3-({[(2,4,6-trimethylphenyl)amino]carbonyl}amino)-2-naphthalenyl]carbonyl}-L-serinate (0.16 g, 0.335 mmol) in dioxane:water/10:1 (8 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.036 g (23% yield) of product as a fluffy tan solid. ES MS m/z 464 (M+H).

### Example 274

### O-(1-methylethyl)-N-{[3-({[(2,4,6-trimethylphenyl)amino]carbonyl}amino)-2-naphthalenyl]carbonyl}-L-serine

### Step 1

### Methyl O-(1-methylethyl)-N-{[(phenylmethyl)oxy]carbonyl}-L-serinate

To a solution of 2-methyl 1-(phenylmethyl) (2S)-1,2-aziridinedicarboxylate (0.5 g, 2.26 mmol) in CHCl₃ (5 ml) was added 2-propanol (6.28 g, 104.49 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 0.6 g of product as a clear oil.

### Step 2

### Methyl O-(1-methylethyl)-L-serinate

Palladium (10% weight on activated carbon, catalytic amount) was added to a solution of methyl O-(1-methylethyl)-N-{[(phenylmethyl)oxy]carbonyl}-L-serinate (0.6 g, 2.03 mmol) in 10 ml of EtOH in a flask under nitrogen. A balloon of H₂ was then affixed to the reaction flask and the reaction was stirred for 2 hours at RT. The reaction was then filtered through a filter paper and the solvent evaporated to give 0.27 g of clear oil.

### Step 3

### Methyl N-[(3-amino-2-naphthalenyl)carbonyl]-O-(l-methylethyl)-L-serinate

HATU (0.53 g, 1.39 mmol) was added to a solution of 3-amino-2-naphthalenecarboxylic acid (0.26 g, 1.39 mmol), methyl O-(1-methylethyl)-L-serinate (0.27 g, 1.67 mmol) and diisopropylethylamine (0.18 g, 1.38 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.23 g of amber oil.

### Step 4

### Methyl O-(1-methylethyl)-N-{[3-({[(2,4,6-trimethylphenyl)amino]carbonyl}amino)-2-naphthalenyl]carbonyl}-L-serinate

Methyl N-[(3-amino-2-naphthalenyl)carbonyl]-O-(1-methylethyl)-L-serinate (0.23 g, 0.696 mmol) in 10 mL of pyridine was treated with 2-isocyanato-1,3,5-trimethylbenzene (0.56 g, 3.47 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.25 g of product as light yellow semi-solid.

### Step 5

### O-(1-methylethyl)-N-{[3-({[(2,4,6-trimethylphenyl)amino]carbonyl}amino)-2-naphthalenyl]carbonyl}-L-serine

Lithium hydroxide monohydrate (0.12 g, 5.01 mmol) was added to a solution of methyl O-(1-methylethyl)-N-{[3-({[(2,4,6-trimethylphenyl)amino]carbonyl}amino)-2-naphthalenyl]carbonyl}-L-serinate (0.25 g, 0.508 mmol) in dioxane:water/10:1 (8 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.088 g (36% yield) of product as a fluffy tan solid. ES MS m/z 478 (M+H).

### Example 275

### O-(2,2-dimethylpropyl)-N-{[3-({[(2,4,6-trimethylphenyl)amino]carbonyl}amino)-2-naphthalenyl]carbonyl}-L-serine

### Step 1

### Methyl O-(2,2-dimethylpropyl)-N-{[(phenylmethyl)oxy]carbonyl}-L-serinate

To a solution of 2-methyl 1-(phenylmethyl) (2S)-1,2-aziridinedicarboxylate (0.5 g, 2.26 mmol) in CHCl₃ (5 ml) was added 2,2-dimethyl-1-propanol (1.87 g, 21.21 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 11.0 g of product as a clear oil.

### Step 2

### Methyl O-(2,2-dimethylpropyl)-L-serinate

Palladium (10% weight on activated carbon, catalytic amount) was added to a solution of methyl O-(2,2-dimethylpropyl)-N-{[(phenylmethyl)oxy]carbonyl}-L-serinate (1.0 g, 3.09 mmol) in 10 ml of EtOH in a flask under nitrogen. A balloon of H₂ was then affixed to the reaction flask and the reaction was stirred for 2 hours at RT. The reaction was then filtered through a filter paper and the solvent evaporated to give 0.47 g of clear oil.

### Step 3

### Methyl N-[(3-amino-2-naphthalenyl)carbonyl]-O-(2,2-dimethylpropyl)-L-serinate

HATU (0.8 g, 2.10 mmol) was added to a solution of 3-amino-2-naphthalenecarboxylic acid (0.39 g, 2.08 mmol), methyl O-(2,2-dimethylpropyl)-L-serinate (0.47 g, 2.48 mmol) and diisopropylethylamine (0.27 g, 2.09 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.51 g of orange solid.

### Step 4

### Methyl O-(2,2-dimethylpropyl)-N-{[3-({[(2,4,6-trimethylphenyl)amino]carbonyl}amino)-2-naphthalenyl]carbonyl}-L-serinate

Methyl N-[(3-amino-2-naphthalenyl)carbonyl]-O-(2,2-dimethylpropyl)-L-serinate (0.51 g, 1.42 mmol) in 10 mL of pyridine was treated with 2-isocyanato-1,3,5-trimethylbenzene (1.15 g, 7.12 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.47 g of product as a cream solid.

### Step 5

### O-(2,2-dimethylpropyl)-N-{[3-({[(2,4,6-trimethylphenyl)amino]carbonyl}amino)-2-naphthalenyl]carbonyl}-L-serine

Lithium hydroxide monohydrate (0.19 g, 7.93 mmol) was added to a solution of methyl O-(2,2-dimethylpropyl)-N-{[3-({[(2,4,6-trimethylphenyl)amino]carbonyl}amino)-2-naphthalenyl]carbonyl}-L-serinate (0.47 g, 0.905 mmol) in dioxane:water/10:1 (8 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.173 g (43% yield) of product as a fluffy tan solid. ES MS m/z 506 (M+H).

### Example 276

### O-(tetrahydro-2H-pyran-4-yl)-N-{[3-({[(2,4,6-trimethylphenyl)amino]carbonyl}amino)-2-naphthalenyl]carbonyl}-L-serine

### Step 1

### Methyl N-{[(phenylmethyl)oxy]carbonyl}-O-(tetrahydro-2H-pyran-4-yl)-L-serinate

To a solution of 2-methyl 1-(phenylmethyl) (2S)-1,2-aziridinedicarboxylate (0.5 g, 2.26 mmol) in CHCl₃ (5 ml) was added tetrahydro-2H-pyran-4-ol (2.30 g, 22.55 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 1.01 g of product as a clear oil.

### Step 2

### Methyl O-(tetrahydro-2H-pyran-4-yl)-L-serinate

Palladium (10% weight on activated carbon, catalytic amount) was added to a solution of methyl N-{[(phenylmethyl)oxy]carbonyl}-O-(tetrahydro-2H-pyran-4-yl)-L-serinate (1.01 g, 3.09 mmol) in 10 ml of EtOH in a flask under nitrogen. A balloon of H₂ was then affixed to the reaction flask and the reaction was stirred for 2 hours at RT. The reaction was then filtered through a filter paper and the solvent evaporated to give 0.63 g of light yellow oil.

### Step 3

### Methyl N-[(3-amino-2-naphthalenyl)carbonyl]-O-(tetrahydro-2H-pyran-4-yl)-L-serinate

HATU (0.99 g, 2.60 mmol) was added to a solution of 3-amino-2-naphthalenecarboxylic acid (0.48 g, 2.56 mmol), methyl O-(tetrahydro-2H-pyran-4-yl)-L-serinate (0.63 g, 3.10 mmol) and diisopropylethylamine (0.33 g, 2.58 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.06 g of amber oil.

### Step 4

### Methyl O-(tetrahydro-2H-pyran-4-yl)-N-{[3-({[(2,4,6-trimethylphenyl)amino]carbonyl}amino)-2-naphthalenyl]carbonyl}-L-serinate

Methyl N-[(3-amino-2-naphthalenyl)carbonyl]-O-(tetrahydro-2H-pyran-4-yl)-L-serinate (0.06 g, 0.16 mmol) in 5 mL of pyridine was treated with 2-isocyanato-1,3,5-trimethylbenzene (0.13 g, 0.804 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.06 g of product as amber oil.

### Step 5

### O-(tetrahydro-2H-pyran-4-yl)-N-{[3-({[(2,4,6-trimethylphenyl)amino]carbonyl}amino)-2-naphthalenyl]carbonyl}-L-serine

Lithium hydroxide monohydrate (0.027 g, 1.13 mmol) was added to a solution of methyl O-(tetrahydro-2H-pyran-4-yl)-N-{[3-({[(2,4,6-trimethylphenyl)amino]carbonyl}amino)-2-naphthalenyl]carbonyl}-L-serinate (0.06 g, 0.112 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo. Purification on Agilent gave 0.012 g (21% yield) of product as a fluffy amber solid. ES MS m/z 520 (M+H).

### Example 277

### O-methyl-N-{[3-({[(2,4,6-trimethylphenyl)amino]carbonyl}amino)-2-naphthalenyl]carbonyl}-L-threonine

### Step 1

### Methyl N-(triphenylmethyl)-L-threoninate

To a cooled (0° C.) solution of methyl L-threoninate hydrochloride (5.0 g, 29.48 mmol) and triethylamine (5.97 g, 58.97 mmol) in chloroform (100 ml) was added trityl chloride as a solid (8.22 g, 29.49 mmol). The reaction was stirred for 12 hours and allowed to come to RT. The reaction was concentrated in vacuo and then dissolved in ethyl acetate and washed with saturated sodium chloride, 10% citric acid, saturated NaHCO₃, and saturated sodium chloride. The organic layer was dried over MgSO₄, filtered and stripped to give 10.16 g of product as a fluffy cream solid. ES MS m/z 398 (M+Na).

### Step 2

### Methyl(2R,3S)-3-methyl-1-(triphenylmethyl)-2-aziridinecarboxylate

To a cooled (0° C.) solution of methyl N-(triphenylmethyl)-L-threoninate (10.16 g, 27.95 mmol) in anhydrous pyridine was added methanesulfonyl chloride (9.61 g, 83.85 mmol) and the reaction was allowed to stir for 12 hours and allowed to come to RT. The solvent was removed in vacuo and the residue dissolved in ethyl acetate. The organic layer was washed with saturated sodium chloride and then dried over MgSO₄, filtered and stripped to 12.33 g of amber oil which was then dissolved in 80 ml of anhydrous THF and to which was added triethylamine (8.50 g, 84.01 mmol) and heated to 80° C. and allowed to reflux for 48 hours. The heat was removed and the reaction was concentrated in vacuo and the residue dissolved in ethyl acetate and washed successively with saturated sodium chloride, 10% citric acid, saturated NaHCO₃ and saturated sodium chloride. The ethyl acetate layer was dried over MgSO₄, filtered and stripped to give 9.04 g of amber oil. Chromatography on silica gel with hexane/ethyl acetate gave 5.26 g of product fluffy cream solid. ES MS m/z 380 (M+Na).

### Step 3

### 2-methyl 1-(phenylmethyl) (2R,3S)-3-methyl-1,2-aziridinedicarboxylate

To a solution of methyl(2R,3S)-3-methyl-1-(triphenylmethyl)-2-aziridinecarboxylate (5.26 g, 14.72 mmol) in CHCl₃ (12 ml) and MeOH (12 ml) cooled to 0° C. was added 11.6 ml of TFA and allowed to stir at 0° C. for 2.5 hours. The reaction was then concentrated in vacuo and evaporated with ether newly added several times to remove TFA. The residue was dissolved in ether which was extracted with water three times. To the aqueous extract at 0° C. was added NaHCO₃ (5.84 g, 69.52 mmol), benzyl chloroformate (2.51 g, 14.71 mmol) and 50 ml of ethyl acetate under vigorous stirring for 1.5 hours. The ethyl acetate layer was separated and the water layer back-extracted. The organics were dried over MgSO₄, filtered and concentrated to give 2.96 g of light yellow oil. Chromatography on silica gel with hexane/ethyl acetate gave 2.45 g of product as a clear oil. ES MS m/z 250 (M+H).

### Step 4

### Methyl O-methyl-N-{[(phenylmethyl)oxy]carbonyl}-L-allothreoninate

To a solution of 2-methyl 1-(phenylmethyl) (2R,3S)-3-methyl-1,2-aziridinedicarboxylate (0.5 g, 2.06 mmol) in CHCl₃ (10 ml) was added methanol (0.64 g, 20.00 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 0.68 g of product as a clear oil.

### Step 5

### Methyl O-methyl-L-allothreoninate

Palladium (10% weight on activated carbon, catalytic amount) was added to a solution of methyl O-methyl-N-{[(phenylmethyl)oxy]carbonyl}-L-allothreoninate (0.68 g, 2.42 mmol) in 10 ml of EtOH in a flask under nitrogen. A balloon of H₂ was then affixed to the reaction flask and the reaction was stirred for 3 hours at RT. The reaction was then filtered through a filter paper and the solvent evaporated to give 0.21 g of clear oil.

### Step 6

### Methyl N-[(3-amino-2-naphthalenyl)carbonyl]-O-methyl-L-threoninate

HATU (0.53 g, 1.39 mmol) was added to a solution of 3-amino-2-naphthalenecarboxylic acid (0.22 g, 1.18 mmol), methyl O-methyl-L-allothreoninate (0.21 g, 1.43 mmol) and diisopropylethylamine (0.18 g, 1.38 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.4 g of amber oil.

### Step 7

### Methyl O-methyl-N-{[3-({[(2,4,6-trimethylphenyl)amino]carbonyl}amino)-2-naphthalenyl]carbonyl}-L-threoninate

Methyl N-[(3-amino-2-naphthalenyl)carbonyl]-O-methyl-L-threoninate (0.4 g, 1.27 mmol) in 10 mL of pyridine was treated with 2-isocyanato-1,3,5-trimethylbenzene (1.02 g, 6.31 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.36 g of product as cream solid.

### Step 8

### O-methyl-N-{[3-({[(2,4,6-trimethylphenyl)amino]carbonyl}amino)-2-naphthalenyl]carbonyl}-L-threonine

Lithium hydroxide monohydrate (0.18 g, 7.52 mmol) was added to a solution of methyl O-methyl-N-{[3-({[(2,4,6-trimethylphenyl)amino]carbonyl}amino)-2-naphthalenyl]carbonyl}-L-threoninate (0.36 g, 0.754 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.124 g (36% yield) of product as a fluffy cream solid. ES MS m/z 464 (M+H).

### Example 278

### O-(1-methylethyl)-N-{[3-({[(2,4,6-trimethylphenyl)amino]carbonyl}amino)-2-naphthalenyl]carbonyl}-L-threonine

### Step 1

### 2-methyl 1-(phenylmethyl) (2R,3S)-3-methyl-1,2-aziridinedicarboxylate

To a solution of methyl(2R,3S)-3-methyl-1-(triphenylmethyl)-2-aziridinecarboxylate (5.26 g, 14.72 mmol) in CHCl₃ (12 ml) and MeOH (12 ml) cooled to 0° C. was added 11.6 ml of TFA and allowed to stir at 0° C. for 2.5 hours. The reaction was then concentrated in vacuo and evaporated with ether newly added several times to remove TFA. The residue was dissolved in ether which was extracted with water three times. To the aqueous extract at 0° C. was added NaHCO₃ (5.84 g, 69.52 mmol), benzyl chloroformate (2.51 g, 14.71 mmol) and 50 ml of ethyl acetate under vigorous stirring for 1.5 hours. The ethyl acetate layer was separated and the water layer back-extracted. The organics were dried over MgSO₄, filtered and concentrated to give 2.96 g of light yellow oil. Chromatography on silica gel with hexane/ethyl acetate gave 2.45 g of product as a clear oil. ES MS m/z 250 (M+H).

### Step 2

### Methyl O-(1-methylethyl)-N-{[(phenylmethyl)oxy]carbonyl}-L-allothreoninate

To a solution of 2-methyl 1-(phenylmethyl) (2R,3S)-3-methyl-1,2-aziridinedicarboxylate (0.5 g, 2.06 mmol) in CHCl₃ (10 ml) was added isopropanol (1.20 g, 19.97 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 0.8 g of product as a clear oil.

### Step 3

### Methyl O-(1-methylethyl)-L-allothreoninate

Palladium (10% weight on activated carbon, catalytic amount) was added to a solution of methyl O-(1-methylethyl)-N-{[(phenylmethyl)oxy]carbonyl}-L-allothreoninate (0.8 g, 2.59 mmol) in 10 ml of EtOH in a flask under nitrogen. A balloon of H₂ was then affixed to the reaction flask and the reaction was stirred for 3 hours at RT. The reaction was then filtered through a filter paper and the solvent evaporated to give 0.28 g of clear oil.

### Step 4

### Methyl N-[(3-amino-2-naphthalenyl)carbonyl]-O-(1-methylethyl)-L-threoninate

HATU (0.61 g, 1.60 mmol) was added to a solution of 3-amino-2-naphthalenecarboxylic acid (0.25 g, 1.34 mmol), methyl O-(1-methylethyl)-L-allothreoninate (0.28 g, 1.60 mmol) and diisopropylethylamine (0.21 g, 1.61 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.27 g of amber oil.

### Step 5

### Methyl O-(1-methylethyl)-N-{[3-({[(2,4,6-trimethylphenyl)amino]carbonyl}amino)-2-naphthalenyl]carbonyl}-L-threoninate

Methyl N-[(3-amino-2-naphthalenyl)carbonyl]-O-(1-methylethyl)-L-threoninate (0.27 g, 0.78 mmol) in 10 mL of pyridine was treated with 2-isocyanato-1,3,5-trimethylbenzene (0.63 g, 3.90 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.39 g of product as amber semi-solid.

### Step 6

### O-(1-methylethyl)-N-{[3-({[(2,4,6-trimethylphenyl)amino]carbonyl}amino)-2-naphthalenyl]carbonyl}-L-threonine

Lithium hydroxide monohydrate (0.185 g, 7.72 mmol) was added to a solution of methyl O-(1-methylethyl)-N-{[3-({[(2,4,6-trimethylphenyl)amino]carbonyl}amino)-2-naphthalenyl]carbonyl}-L-threoninate (0.39 g, 0.771 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.067 g (18% yield) of product as a fluffy cream solid. ES MS m/z 492 (M+H).

### Example 279

### 1-({[3-({[(2,4,6-Trichlorophenyl)amino]carbonyl}amino)-2-naphthalenyl]carbonyl}amino)cyclopentanecarboxylic acid

### Step 1

### Methyl 1-{[(3-amino-2-naphthalenyl)carbonyl]amino}cyclopentanecarboxylate

3-Amino-2-naphthoic acid (0.2 g, 1.0 mmol) and methyl 1-aminocyclopentanecarboxylate hydrochloride (0.21 g, 1.17 mmol) were dissolved in DMF (10 mL) and diisopropylethylamine (0.41 g, 3.20 mmol) and HATU (0.45 g, 1.17 mmol) were added. The solution was heated to 50° C. for 1 h and stirred overnight. The reaction was diluted with water and extracted with ethyl acetate. The extracts were washed with brine, dried (MgSO₄) and concentrated onto SiO₂. Chromatography on SiO₂ eluting with ethyl acetate/hexanes provided 0.17 g of product as a yellow solid.

### Step 2

### Methyl 1-({[3-({[(2,4,6-trichlorophenyl)amino]carbonyl}amino)-2-naphthalenyl]carbonyl}amino)cyclopentanecarboxylate

Methyl 1-{[(3-amino-2-naphthalenyl)carbonyl]amino}cyclopentanecarboxylate (47 mg, 0.15 mmol) was dissolved in DMF (1 mL) and triethylamine (30 mg, 0.30 mmol) and 2,4,6-trichlorophenyl isocyanate (40 mg, 0.18 mmol) were added. The reaction was heated to 70° C. for 2 h and cooled. The reaction was diluted with water and extracted with ethyl acetate. The extracts were dried (MgSO₄) and concentrated onto SiO₂. Chromatography on SiO₂ eluting with ethyl acetate/hexanes provided 50 mg of product.

### Step 3

### 1-({[3-({[(2,4,6-Trichlorophenyl)amino]carbonyl}amino)-2-naphthalenyl]carbonyl}amino)cyclopentanecarboxylic acid

Methyl 1-({[3-({[(2,4,6-trichlorophenyl)amino]carbonyl}amino)-2-naphthalenyl]carbonyl}amino)cyclopentanecarboxylate (50 mg, 0.093 mmol) was dissolved in 1:1 THF/MeOH (1 mL) and 1 M NaOH (0.47 mL) was added. The solution was heated to 50° C. for 2 h and cooled. The reaction was diluted with water, acidified with 1 M HCl (0.5 mL), and extracted with ethyl acetate. The extracts were dried (MgSO₄) and concentrated to afford 46 mg of product as a tan solid. ES MS m/z 521 (M+H).

### Example 280

### 1-({[3-({[(2,4,6-trimethylphenyl)amino]carbonyl}amino)-2-naphthalenyl]carbonyl}amino)cyclohexanecarboxylic acid

### Step 1

### Methyl 1-{[(3-amino-2-naphthalenyl)carbonyl]amino}cyclohexanecarboxylate

Methyl 1-aminocyclohexanecarboxylate hydrochloride (0.28 g, 1.45 mmol) and 3-amino-2-naphthoic acid (0.3 g, 1.60 mmol) were dissolved in DMF (10 mL) and diisopropylethylamine (0.56 g, 4.33 mmol) and HATU (0.60 g, 1.59 mmol) were added. The reaction was heated to 50° C. for ca. 30 min and cooled. The reaction was diluted with ethyl acetate and washed with water and brine. The organic layer was dried (MgSO₄) and concentrated onto SiO₂. Chromatography on SiO₂ eluting with ethyl acetate/hexanes provided 0.42 g of product as a yellow solid.

### Step 2

### 1-{[(3-amino-2-naphthalenyl)carbonyl]amino}cyclohexanecarboxylic acid

Methyl 1-{[(3-amino-2-naphthalenyl)carbonyl]amino}cyclohexanecarboxylate (0.42 g, 1.28 mmol) was dissolved in THF (5 mL) and MeOH (5 mL) and 5 M NaOH (2.6 mL, 13 mmol) and water (2.5 mL) were added. The solution was heated to 55° C. for ca. 3 h and cooled. The solution was acidified with 5 M HCl (3 mL), diluted with water, and extracted with ethyl acetate. The extracts were dried (MgSO₄) and concentrated to afford 0.23 g of product as a gold solid.

### Step 3

### 1-({[3-({[(2,4,6-trimethylphenyl)amino]carbonyl}amino)-2-naphthalenyl]carbonyl}amino)cyclohexanecarboxylic acid

1-{[(3-Amino-2-naphthalenyl)carbonyl]amino}cyclohexanecarboxylic acid (0.1 g, 0.32 mmol) was dissolved in DMF (3 mL) and 2,4,6-trimethylphenyl isocyanate (57 mg, 0.35 mmol) and triethylamine (65 mg, 0.64 mmol) were added. The solution was heated to 70° C. for ca. 90 min and cooled. The reaction was diluted with water and 5 M HCl (1 mL) was added. The solution was extracted with ethyl acetate and the extracts were dried (MgSO₄) and concentrated onto SiO₂. Chromatography on SiO₂ eluting with ethyl acetate/hexanes afforded 97 mg of product as a gold solid. ES MS m/z 474 (M+H).

### Example 281

### 1-({[3-({[(2,4,6-trichlorophenyl)amino]carbonyl}amino)-2-naphthalenyl]carbonyl}amino)cyclohexanecarboxylic acid

### Step 1

### 1-({[3-({[(2,4,6-trichlorophenyl)amino]carbonyl}amino)-2-naphthalenyl]carbonyl}amino)cyclohexanecarboxylic acid

1-{[(3-Amino-2-naphthalenyl)carbonyl]amino}cyclohexanecarboxylic acid (50 mg, 0.16 mmol) was dissolved in DMF (1 mL) and 2,4,6-trichlorophenyl isocyanate (39 mg, 0.17 mmol) and triethylamine (33 mg, 0.32 mmol) were added. The solution was heated to 70° C. for ca. 90 min and stirred overnight. The reaction was diluted with water and 5 M HCl (1 mL) was added. The solution was extracted with ethyl acetate and the extracts were dried (MgSO₄) and concentrated onto SiO₂. Chromatography on SiO₂ eluting with ethyl acetate/hexanes afforded 45 mg of product as a gold solid. ES MS m/z 534 (M+H).

### Example 282

### 4-({[3-({[(2,4,6-Trimethylphenyl)amino]carbonyl}amino)-2-naphthalenyl]carbonyl}amino)tetrahydro-2H-pyran-4-carboxylic acid

### Step 1

### Methyl 4-({[(1,1-dimethylethyl)oxy]carbonyl}amino)tetrahydro-2H-pyran-4-carboxylate

4-({[(1,1-Dimethylethyl)oxy]carbonyl}amino)tetrahydro-2H-pyran-4-carboxylic acid (0.5 g, 2.03 mmol) was dissolved in MeOH (6 mL) and the solution was cooled to 0° C. Trimethylsilyidiazomethane (3.5 mL of a 2 M solution) was added dropwise until a yellow color persisted and the reaction was stirred for 60 min and concentrated to afford 0.52 g of product as a viscous oil.

### Step 2

### Methyl 4-aminotetrahydro-2H-pyran-4-carboxylate

Methyl 4-({[(1,1-dimethylethyl)oxy]carbonyl}amino)tetrahydro-2H-pyran-4-carboxylate (0.52 g, 1.69 mmol) was dissolved in CH₂Cl₂ (10 mL) and TFA (0.75 mL) was added. The mixture was stirred overnight and concentrated to afford product as a viscous oil which was used without further purification.

### Step 3

### Methyl 4-{[(3-amino-2-naphthalenyl)carbonyl]amino}tetrahydro-2H-pyran-4-carboxylate

3-Amino-2-napthoic acid (0.18 g, 0.80 mmol) was dissolved in DMF (3 mL) and diisopropylethylamine (0.28 g, 2.2 mmol) and HATU (0.31 g, 0.80 mmol) were added. The solution was stirred 20 min and methyl 4-aminotetrahydro-2H-pyran-4-carboxylate (0.20 g, 0.73 mmol) was added. The reaction was heated to 500° C. for 1 h, cooled, diluted with water, and extracted with ethyl acetate. The extracts were dried (MgSO₄) and concentrated onto SiO₂. Chromatography on SiO₂ eluting with ethyl acetate/hexanes afforded 0.13 g of product.

### Step 4

### Methyl 4-({[3-({[(2,4,6-trimethylphenyl)amino]carbonyl}amino)-2-naphthalenyl]carbonyl}amino)tetrahydro-2H-pyran-4-carboxylate

Methyl 4-{[(3-amino-2-naphthalenyl)carbonyl]amino}tetrahydro-2H-pyran-4-carboxylate (76 mg, 0.23 mmol) was dissolved in pyridine (1 mL) and 2,4,6-trimethylphenyl isocyanate (0.19 g, 1.15 mmol) was added. The reaction was stirred 6 h and then diluted with ethyl acetate and concentrated onto SiO₂. Chromatography on SiO₂ eluting with ethyl acetate/hexanes afforded 100 mg of product.

### Step 5

### 4-({[3-({[(2,4,6-Trimethylphenyl)amino]carbonyl}amino)-2-naphthalenyl]carbonyl}amino)tetrahydro-2H-pyran-4-carboxylic acid

Methyl 4-({[3-({[(2,4,6-trimethylphenyl)amino]carbonyl}amino)-2-naphthalenyl]carbonyl}amino)tetrahydro-2H-pyran-4-carboxylate (110 mg, 0.22 mmol) was dissolved in 1:1 THF/MeOH (2 mL) and 2 M LiOH (1.1 mL) was added. The reaction was heated to 500° C. for 1 h, acidified with 1 M HCl (2.2 mL), and extracted with ethyl acetate. The extracts were dried (MgSO4), concentrated, redissolved in diethyl ether, and reconcentrated to afford 100 mg of product as a foam. ES MS m/z 476 (M+H)

### Example 283

### 4-({[3-({[(2,6-Dichlorophenyl)amino]carbonyl}amino)-2-naphthalenyl]carbonyl}amino)tetrahydro-2H-pyran-4-carboxylic acid

### Step 1

### Methyl 4-({[3-({[(2,6-dichlorophenyl)amino]carbonyl}amino)-2-naphthalenyl]carbonyl}amino)tetrahydro-2H-pyran-4-carboxylate

1 (47 mg, 0.14 mmol) was dissolved in pyridine (1 mL) and 2,6-dichlorophenyl isocyanate (0.13 g, 0.71 mmol) was added. The reaction was stirred 90 min and concentrated onto SiO₂. Chromatography on SiO₂ eluting with ethyl acetate/hexanes afforded 46 mg of product.

### Step 2

### 4-({[3-({[(2,6-Dichlorophenyl)amino]carbonyl}amino)-2-naphthalenyl]carbonyl}amino)tetrahydro-2H-pyran-4-carboxylic acid

Methyl 4-({[3-({[(2,6-dichlorophenyl)amino]carbonyl}amino)-2-naphthalenyl]carbonyl}amino)tetrahydro-2H-pyran-4-carboxylate (56 mg, 0.22 mmol) was dissolved in 1:1 THF/MeOH (mL) and 2 M LiOH (0.54 mL) was added. The reaction was heated to 500° C. for 1 h, cooled, diluted with ethyl acetate and acidified with 1 M HCl (2.2 mL). The organic layer was dried (MgSO₄) and concentrated. The residue was taken up in CH₂Cl₂ and a colorless solid formed. The solid was dried under vacuum to provide 36 mg of product. ES MS m/z 502 (M+H)

### Example 284

### 4-({[3-({[(2,4,6-Trichlorophenyl)amino]carbonyl}amino)-2-naphthalenyl]carbonyl}amino)tetrahydro-2H-thiopyran-4-carboxylic acid

### Step 1

### Methyl 4-aminotetrahydro-2H-thiopyran-4-carboxylate hydrochloride

4-Aminotetrahydro-2H-thiopyran-4-carboxylic acid hydrochloride (0.5 g, mmol) was dissolved in MeOH (20 mL) and HCl (g) was bubbled through the solution for 20 min. The solution was heated to reflux for 4 h, cooled, and concentrated afford product as a colorless solid which was used without further purification.

### Step 2

### Methyl 4-{[(3-amino-2-naphthalenyl)carbonyl]amino}tetrahydro-2H-thiopyran-4-carboxylate

3-Amino-2-naphthoic acid (0.3 g, 1.36 mmol) was dissolved in DMF (5 mL) and diisopropylethylamine (0.53 g, 4.08 mmol) and HATU (0.57 g, 1.50 mmol) were added and stirred for 15 min. Methyl 4-aminotetrahydro-2H-thiopyran-4-carboxylate hydrochloride (0.35 g, 1.63 mmol) was added and the mixture was stirred overnight. The reaction was diluted with ethyl acetate, washed with water, dried (MgSO₄), and concentrated onto SiO₂. Chromatography on SiO₂ eluting with ethyl acetate/hexanes afforded 92 mg of product.

### Step 3

### Methyl 4-({[3-({[(2,4,6-trichlorophenyl)amino]carbonyl}amino)-2-naphthalenyl]carbonyl}amino)tetrahydro-2H-thiopyran-4-carboxylate

Methyl 4-{[(3-amino-2-naphthalenyl)carbonyl]amino}tetrahydro-2H-thiopyran-4-carboxylate (92 mg, 0.26 mmol) was dissolved in DMF (2 mL) and triethylamine (81 mg, 0.11 mL) and 2,4,6-trichlorophenyl isocyanate (0.12 g, 0.53 mmol) was added. The reaction was heated to 600° C. for 4 h and then stirred at RT overnight. The reaction was diluted with water and extracted with ethyl acetate. The extracts were dried (MgSO₄) and concentrated onto SiO₂. Chromatography on SiO₂ eluting with ethyl acetate/hexanes afforded 30 mg of product.

### Step 4

### 4-({[3-({[(2,4,6-Trichlorophenyl)amino]carbonyl}amino)-2-naphthalenyl]carbonyl}amino)tetrahydro-2H-thiopyran-4-carboxylic acid

Methyl 4-({[3-({[(2,4,6-trichlorophenyl)amino]carbonyl}amino)-2-naphthalenyl]carbonyl}amino)tetrahydro-2H-thiopyran-4-carboxylate (30 mg, 0.053 mmol) was dissolved in 1:1 THF/MeOH (2 mL) and 2 M LiOH (0.26 mL) was added. The reaction was heated to 600° C. for 4 h, cooled, diluted with water, and acidified with 1 M HCl (0.55 mL). The mixture was extracted with ethyl acetate and the extracts were dried (MgSO₄) and concentrated. The residue was dissolved in MeOH (1 mL) and purified on the reverse-phase HPLC to afford 20 mg of product. MS m/z 553 (M+H)

### Example 285

### 4-({[3-({[(2,4,6-Trimethylphenyl)amino]carbonyl}amino)-2-naphthalenyl]carbonyl}amino)tetrahydro-2H-thiopyran-4-carboxylic acid 1,1-dioxide

### Step 1

### Methyl 4-({[(1,1-dimethylethyl)oxy]carbonyl}amino)tetrahydro-2H-thiopyran-4-carboxylate 1,1-dioxide

4-({[(1,1-Dimethylethyl)oxy]carbonyl}amino)tetrahydro-2H-thiopyran-4-carboxylic acid 1,1-dioxide (0.5 g) was suspended in MeOH (6 mL) and cooled to 0° C. Trimethylsilyidiazomethane (4 mL of a 2 M solution) was added dropwise and stirred 60 min. The reaction was concentrated to afford 0.52 g of product.

### Step 2

### 4-Aminotetrahydro-2H-thiopyran-4-carboxylic acid 1,1-dioxide trifluoroacetate

Methyl 4-({[(1,1-dimethylethyl)oxy]carbonyl}amino)tetrahydro-2H-thiopyran-4-carboxylate 1,1-dioxide (0.52 g, 1.69 mmol) was dissolved in CH₂Cl₂ and TFA (0.75 mL) was added and the reaction was stirred for 15 h. The solution was concentrated to afford product as a solid.

### Step 3

### Methyl 4-{[(3-amino-2-naphthalenyl)carbonyl]amino}tetrahydro-2H-thiopyran-4-carboxylate 1,1-dioxide

3-Amino-2-naphthoic acid (0.19 g, 0.85 mmol) was dissolved in DMF (3 mL) and diisopropylethylamine (0.30 g, 0.41 mmol) and HATU (0.32 g, 0.85 mmol) were added and stirred for 15 min. 4-Aminotetrahydro-2H-thiopyran-4-carboxylic acid 1,1-dioxide trifluoroacetate (0.25 g, 0.78 mmol) was added and the mixture was heated to 50° C. for 60 min and cooled. Water was added and the mixture was extracted with ethyl acetate. The extracts were dried (MgSO₄) and concentrated onto SiO₂. Chromatography on SiO₂ eluting with ethyl acetate/hexanes afforded 150 mg of product.

### Step 4

### Methyl 4-({[3-({[(2,4,6-trimethylphenyl)amino]carbonyl}amino)-2-naphthalenyl]carbonyl}amino)tetrahydro-2H-thiopyran-4-carboxylate 1,1-dioxide

Methyl 4-{[(3-amino-2-naphthalenyl)carbonyl]amino}tetrahydro-2H-thiopyran-4-carboxylate 1,1-dioxide (0.14 g, 0.37 mmol) was dissolved in pyridine (5 mL) and 2,4,6-trimethylphenylisocyanate (0.30 g, 1.86 mmol) was added. The reaction was stirred 6 h, diluted with ethyl acetate, and concentrated onto SiO₂. Chromatography on SiO₂ eluting with ethyl acetate/hexanes provided 0.50 g of product as a solid.

### Step 5

### 4-({[3-({[(2,4,6-Trimethylphenyl)amino]carbonyl}amino)-2-naphthalenyl]carbonyl}amino)tetrahydro-2H-thiopyran-4-carboxylic acid 1,1-dioxide

Methyl 4-({[3-({[(2,4,6-trimethylphenyl)amino]carbonyl}amino)-2-naphthalenyl]carbonyl}amino)tetrahydro-2H-thiopyran-4-carboxylate 1,1-dioxide (136 mg, 0.25 mmol) was dissolved in 1:1 THF/MeOH (1 mL) and 2 M LiOH (1.26 mL) was added. The reaction was heated to 500° C. for 1 h and stirred overnight. The reaction was acidified with 1 M HCl (2.5 mL). The mixture was extracted with ethyl acetate and the extracts were dried (MgSO₄) and concentrated. The resulting solid was triturated with CH₂Cl₂ to provide 104 mg of product. MS m/z 524 (M+H)

### Example 286

### 4-({[3-({[(2,6-Dichlorophenyl)amino]carbonyl}amino)-2-naphthalenyl]carbonyl}amino)-1-(phenylmethyl)-4-piperidinecarboxylic acid

### Step 1

### 8-(Phenylmethyl)-1,3,8-triazaspiro[4.5]decane-2,4-dione

1-(Phenylmethyl)-4-piperidinone (10.2 g, 53.9 mmol) was added to a suspension of NaCN (7.1 g, 144 mmol) and (NH₄)₂CO₃ (49.6 g, 518 mmol) in 1:1EtOH/water (140 mL). The mixture was heated to 60° C. overnight and cooled. The solution was allowed to stand 2 days and the resulting solid was filtered off and dried under vacuum to afford 13.0 g of product.

### Step 2

### 4-Amino-1-(phenylmethyl)-4-piperidinecarboxylic acid

8-(Phenylmethyl)-1,3,8-triazaspiro[4.5]decane-2,4-dione (13.0 g, 50.1 mmol)was suspended in water (160 mL) and LiOH (6.0 g, 250 mmol) was added. The solution was heated to reflux for 48 h and cooled. The solution was filtered and the filtrate was concentrated to a residue and the pH was adjusted to 5 by addition of conc HCl. The resulting solid was filtered, suspended in MeOH, refiltered and dried under vacuum to afford 8 g of product.

### Step 3

### Ethyl 4-amino-1-(phenylmethyl)-4-piperidinecarboxylate

4-Amino-1-(phenylmethyl)-4-piperidinecarboxylic acid (4 g, 17 mmol) was suspended in EtOH (40 mL) and the solution was cooled to 0° C. and SOCl₂ (7.5 mL) was added. The solution was warmed to RT and heated to reflux for 5 h. The solution was concentrated to an oil, which was dissolved in water and neutralized to pH=7 with 1 M NaOH and extracted with CH₂Cl₂. The extracts were dried (MgSO₄) and concentrated to afford 0.78 g of product as an oil.

### Step 4

### Ethyl 4-{[(3-amino-2-naphthalenyl)carbonyl]amino}-1-(phenylmethyl)-4-piperidinecarboxylate

3-Amino-2-napthoic acid (0.66 g, 3.01 mmol) was dissolved in DMF (8 mL) and diisopropylethylamine (0.89 g, 6.86 mmol) and HATU (1.14 g, 3.01 mmol) were added and stirred 15 min. Ethyl 4-amino-1-(phenylmethyl)-4-piperidinecarboxylate (0.72 g, 2.74 mmol) was dissolved in DMF (2 mL) and the solution was added to the reaction and heated to 50° C. for 45 min and cooled. The mixture was diluted with ethyl acetate and washed with water. The extracts were dried (MgSO₄) and concentrated onto SiO₂. Chromatography on SiO₂ eluting with ethyl acetate/hexanes provided 1.07 g of product.

### Step 5

### Ethyl 4-({[3-({[(2,6-dichlorophenyl)amino]carbonyl}amino)-2-naphthalenyl]carbonyl}amino)-1-(phenylmethyl)-4-piperidinecarboxylate

Ethyl 4-{[(3-amino-2-naphthalenyl)carbonyl]amino}-1-(phenylmethyl)-4-piperidinecarboxylate (40 mg, 0.092 mmol) was dissolved in pyridine (1.5 mL) and 2,6-dichlorophenylisocyanate (87 mg, 0.46 mmol) was added. The reaction was stirred overnight, diluted with ethyl acetate, and concentrated onto SiO₂. Chromatography on SiO2 eluting with ethyl acetate/hexanes provided 50 mg of product as a solid.

### Step 6

### 4-({[3-({[(2,6-Dichlorophenyl)amino]carbonyl}amino)-2-naphthalenyl]carbonyl}amino)-1-(phenylmethyl)-4-piperidinecarboxylic acid

Ethyl 4-({[3-({[(2,6-dichlorophenyl)amino]carbonyl}amino)-2-naphthalenyl]carbonyl}amino)-1-(phenylmethyl)-4-piperidinecarboxylate (40 mg, 0.08 mmol) was dissolved in 1:1 THF/MeOH (2 mL) and 2 M LiOH (0.4 mL) was added. The reaction was heated to 60° C. overnight, cooled, acidified with 1 M HCl (0.9 mL) and a solid formed. The solid was filtered off, dissolved in MeOH (1 mL), and purified by reverse-phase HPLC to afford 13 mg of product ES MS m/z 492 (M+H)

### Example 287

### 1-{[(1,1-Dimethylethyl)oxy]carbonyl}-4-({[3-({[(2,4,6-trimethylphenyl)amino]carbonyl}amino)-2-naphthalenyl]carbonyl}amino)-4-piperidinecarboxylic acid

### Step 1

### 1-(1,1-Dimethylethyl) 4-methyl 4-({[(9H-fluoren-9-ylmethyl)oxy]carbonyl}amino)-1,4-piperidinedicarboxylate

1-{[(1,1-Dimethylethyl)oxy]carbonyl}-4-({[(9H-fluoren-9-ylmethyl)oxy]carbonyl}amino)-4-piperidinecarboxylic acid (1 g, 2.14 mmol) was dissolved in MeOH (9 mL) and the solution was cooled to 0° C. A solution of TMSCHN₂ (6 mL of a 2 M solution) was added dropwise and the reaction was stirred overnight. The solution was concentrated to provide 1.0 g of product.

### Step 2

### 1-(1,1-Dimethylethyl) 4-methyl 4-amino-1,4-piperidinedicarboxylate

1-(1,1-Dimethylethyl) 4-methyl 4-({[(9H-fluoren-9-ylmethyl)oxy]carbonyl}amino)-1,4-piperidinedicarboxylate (1.0 g, 2.1 mmol) was dissolved in dioxane and polymer supported piperidine (2.1 g of PL-PPZ (5 mmol/g loading)) was added and stirred 24 h at RT and then heated to 50° C. for an additional 15 h. The solution was cooled, filtered, and concentrated to afford an oil which was used in the next step without purification.

### Step 3

### 1-(1,1-Dimethylethyl) 4-methyl 4-{[(3-amino-2-naphthalenyl)carbonyl]amino}-1,4-piperidinedicarboxylate

3-Amino-2-napthoic acid (0.43 g, 2.3 mmol) was dissolved in DMF (8 mL) and diisopropylethylamine (0.89 g, 6.86 mmol) and HATU (1.14 g, 3.01 mmol) were added and stirred 20 min. 1-(1,1-Dimethylethyl) 4-methyl 4-amino-1,4-piperidinedicarboxylate (0.54 g, 2.1 mmol) was dissolved in DMF (2 mL) and the solution was added to the reaction and heated to 50° C. for 60 min and cooled. The mixture was poured onto water and extracted with ethyl acetate. The extracts were washed with brine, dried (MgSO₄) and concentrated onto SiO₂. Chromatography on SiO₂ eluting with ethyl acetate/hexanes provided 0.75 g of product.

### Step 4

### 1-(1,1-Dimethylethyl) 4-methyl 4-({[3-({[(2,4,6-trimethylphenyl)amino]carbonyl}amino)-2-naphthalenyl]carbonyl}amino)-1,4-piperidinedicarboxylate

1-(1,1-Dimethylethyl) 4-methyl 4-{[(3-amino-2-naphthalenyl)carbonyl]amino}-1,4-piperidinedicarboxylate (0.4 g, 0.93 mmol) was dissolved in pyridine (5 mL) and 2,4,6-trimethylphenylisocyanate (0.75 g, 4.68 mmol) was added. The reaction was stirred overnight, diluted with ethyl acetate, and concentrated onto SiO₂. Chromatography on SiO2 eluting with ethyl acetate/hexanes provided 0.50 g of product as a solid.

### Step 5

### 1-{[(11,1-Dimethylethyl)oxy]carbonyl}-4-({[3-({[(2,4,6-trimethylphenyl)amino]carbonyl}amino)-2-naphthalenyl]carbonyl}amino)-4-piperidinecarboxylic acid

1-(1,1-Dimethylethyl) 4-methyl 4-({[3-({[(2,4,6-trimethylphenyl)amino]carbonyl}amino)-2-naphthalenyl]carbonyl}amino)-1,4-piperidinedicarboxylate (50 mg, 0.085 mmol) was dissolved in 1:1 THF/MeOH (1 mL) and 2 M LiOH (0.42 mL) was added. The reaction was heated to 55° C. for 2 h, cooled, acidified with 1 M HCl (0.84 mL) and extracted with ethyl acetate. The extracts were dried (MgSO₄), concentrated, redissolved in CH₂Cl₂, filtered and concentrated to afford 39 mg of product as a tan foam. ES MS m/z 575 (M+H)

### Example 288

### 4-({[3-({[(2,4,6-trimethylphenyl)amino]carbonyl}amino)-2-naphthalenyl]carbonyl}amino)-4-piperidinecarboxylic acid trifluoroacetate

### Step 1

### Methyl 4-({[3-({[(2,4,6-trimethylphenyl)amino]carbonyl}amino)-2-naphthalenyl]carbonyl}amino)-4-piperidinecarboxylate trifluroroacetate

1-(1,1-Dimethylethyl) 4-methyl 4-({[3-({[(2,4,6-trimethylphenyl)amino]carbonyl}amino)-2-naphthalenyl]carbonyl}amino)-1,4-piperidinedicarboxylate (0.44 g, 0.75 mmol) was dissolved in CH₂Cl₂ (5 mL) and TFA (0.5 mL) was added and stirred overnight. The solution was concentrated to provide product as a solid which was used without further purification.

### Step 2

### 4-({[3-({[(2,4,6-trimethylphenyl)amino]carbonyl}amino)-2-naphthalenyl]carbonyl}amino)-4-piperidinecarboxylic acid trifluoroacetate

Methyl 4-({[3-({[(2,4,6-trimethylphenyl)amino]carbonyl}amino)-2-naphthalenyl]carbonyl}amino)-4-piperidinecarboxylate trifluoroacetate (50 mg, 0.083 mmol) was dissolved in 1:1 THF/MeOH (0.8 mL) and 2 M LiOH (0.42 mL) was added. The reaction was heated to 55° C. for 2.5 h, cooled, acidified with 1 M HCl (0.84 mL) and extracted with ethyl acetate. The extracts were dried (MgSO₄), concentrated, and redissolved in MeOH. The solution was purified by reverse-phase HPLC to afford 13 mg of product as the trifluoroacetate salt. ES MS m/z 475 (M+H).

### Example 289

### 1-Butyl-4-({[3-({[(2,4,6-trimethylphenyl)amino]carbonyl}amino)-2-naphthalenyl]carbonyl}amino)-4-piperidinecarboxylic acid

### Step 1

### Methyl 1-butyl-4-({[3-({[(2,4,6-trimethylphenyl)amino]carbonyl}amino)-2-naphthalenyl]carbonyl}amino)-4-piperidinecarboxylate

Methyl 4-({[3-({[(2,4,6-trimethylphenyl)amino]carbonyl}amino)-2-naphthalenyl]carbonyl}amino)-4-piperidinecarboxylate trifluroroacetate (57 mg, 0.095 mmol) was dissolved in DMF (1 mL) and K₂CO₃ (39 mg, 0.28 mmol) and n-butylbromide (19 mg, 0.14 mmol) were added and the reaction was heated to 50° C. overnight and cooled. The reaction was diluted with water and a solid precipitated which was dissolved in ethyl acetate. The aqueous was extracted with ethyl acetate and the combined organics were dried (MgSO₄) and concentrated to afford 50 mg of product.

### Step 2

### 1-Butyl-4-({[3-({[(2,4,6-trimethylphenyl)amino]carbonyl}amino)-2-naphthalenyl]carbonyl}amino)-4-piperidinecarboxylic acid

Methyl 1-butyl-4-({[3-({[(2,4,6-trimethylphenyl)amino]carbonyl}amino)-2-naphthalenyl]carbonyl}amino)-4-piperidinecarboxylate (50 mg, 0.092 mmol) was dissolved in 1:1 THF/MeOH (1 mL) and 2 M LiOH (0.46 mL) was added. The reaction was heated to 50° C. for 4 h, cooled, and stirred overnight. The solution was acidified with 1 M HCl (0.9 mL) and extracted with ethyl acetate. The extracts were dried (MgSO₄) and concentrated to afford 31 mg of product. ES MS m/z 531 (M+H).

### Example 290

### 1-Butanoyl-4-({[3-({[(2,4,6-trimethylphenyl)amino]carbonyl}amino)-2-naphthalenyl]carbonyl}amino)-4-piperidinecarboxylic acid

### Step 1

### Methyl 1-butanoyl-4-({[3-({[(2,4,6-trimethylphenyl)amino]carbonyl}amino)-2-naphthalenyl]carbonyl}amino)-4-piperidinecarboxylate

Methyl 4-({[3-({[(2,4,6-trimethylphenyl)amino]carbonyl}amino)-2-naphthalenyl]carbonyl}amino)-4-piperidinecarboxylate trifluroroacetate (50 mg, 0.083 mmol) was dissolved in CH₂Cl₂ (1.5 mL) and diisopropylethylamine (10 mg, 0.091 mmol) was added followed by butyryl chloride (10 mg, 0.091 mmol). The solution was stirred 15 h and then concentrated onto SiO₂ and purified by chromatography on SiO2 eluting with ethyl acetate/hexanes to afford 32 mg of product.

### Step 2

### 1-Butanoyl-4-({[3-({[(2,4,6-trimethylphenyl)amino]carbonyl}amino)-2-naphthalenyl]carbonyl}amino)-4-piperidinecarboxylic acid

Methyl 1-butanoyl-4-({[3-({[(2,4,6-trimethylphenyl)amino]carbonyl}amino)-2-naphthalenyl]carbonyl}amino)-4-piperidinecarboxylate (32 mg, 0.057 mmol) was dissolved in 1:1 THF/MeOH (1 mL) and 2 M LiOH (0.29 mL) was added. The reaction was heated to 50° C. for 30 min, cooled, acidified with 1 M HCl (0.6 mL) and extracted with ethyl acetate. The extracts were dried (MgSO₄) and concentrated to afford 31 mg of a foam. ES MS m/z 545 (M+H).

### Example 291

### 1-[(Ethyloxy)carbonyl]-4-({[3-({[(2,4,6-trimethylphenyl)amino]carbonyl}amino)-2-naphthalenyl]carbonyl}amino)-4-piperidinecarboxylic acid

### Step 1

### 1-Ethyl 4-methyl 4-({[3-({[(2,4,6-trimethylphenyl)amino]carbonyl}amino)-2-naphthalenyl]carbonyl}amino)-1,4-piperidinedicarboxylate

Methyl 4-({[3-({[(2,4,6-trimethylphenyl)amino]carbonyl}amino)-2-naphthalenyl]carbonyl}amino)-4-piperidinecarboxylate trifluroroacetate (50 mg, 0.083 mmol) was dissolved in CH₂Cl₂ (1.5 mL) and diisopropylethylamine (10 mg, 0.091 mmol) was added followed by ethyl chloroformate (10 mg, 0.091 mmol). The solution was stirred 15 h and then concentrated onto SiO₂ and purified by chromatography on SiO₂ eluting with ethyl acetate/hexanes to afford 31 mg of product.

### Step 2

### 1-[(Ethyloxy)carbonyl]-4-({[3-({[(2,4,6-trimethylphenyl)amino]carbonyl}amino)-2-naphthalenyl]carbonyl}amino)-4-piperidinecarboxylic acid

1-Ethyl 4-methyl 4-({[3-({[(2,4,6-trimethylphenyl)amino]carbonyl}amino)-2-naphthalenyl]carbonyl}amino)-1,4-piperidinedicarboxylate (31 mg, 0.055 mmol) was dissolved in 1:1 THF/MeOH (1 mL) and 2 M LiOH (0.29 mL) was added. The reaction was heated to 50° C. for 30 min, cooled, acidified with 1 M HCl (0.6 mL) and extracted with ethyl acetate. The extracts were dried (MgSO₄) and concentrated to afford 31 mg of a foam. ES MS m/z 547 (M+H).

### Example 292

### 1-({[3-({[(2,6-dichlorophenyl)amino]carbonyl}amino)-2-naphthalenyl]carbonyl}amino)-4-oxocyclohexanecarboxylic acid

### Step 1

### Methyl 2-({[(phenylmethyl)oxy]carbonyl}amino)-2-propenoate

Methyl O-[(4-methylphenyl)sulfonyl]-N-{[(phenylmethyl)oxy]carbonyl}serinate (11 g, 27 mmol) was dissolved in CHCl₃ (70 mL) and triethylamine (5.46 g, 54 mmol) was added in one portion. The solution was stirred overnight and then concentrated and the residue was resuspended in Et₂O. The solution was cooled to 0° C. and the precipitated solid was filtered off. The filtrate was concentrated, redissolved in CHCl₃, washed with 1 M HCl and water, dried (MgSO₄), and concentrated to provide product as an oil which was immediately used in the next reaction.

### Step 2

### Methyl 4-oxo-1-({[(phenylmethyl)oxy]carbonyl}amino)-2-cyclohexene-1-carboxylate

Methyl 2-({[(phenylmethyl)oxy]carbonyl}amino)-2-propenoate (6.3 g, 26.7 mmol) and Danishefsky's diene (9.3 g, 53.5 mmol) were dissolved in toluene (100 mL) and heated to reflux for 5 days. The solution was cooled, concentrated, and redissolved in THF (75 mL). 1 M HCl (25 mL) was added and the mixture was stirred 15 h and concentrated. The residue was redissolved in CH₂Cl₂ and concentrated onto SiO2. Chromatography on SiO2 eluting with ethyl acetate/hexanes afforded 4.4 g of an oil. The oil was dissolved in CH₂Cl₂ (100 mL) and DBU (1.5 g, 9.8 mmol) was added. The reaction was stirred overnight and then washed with saturated NaHCO₃ solution, dried (MgSO₄) and concentrated onto SiO2. Chromatography on SiO2 eluting with ethyl acetate/hexanes provided 3.9 g of product as a clear oil.

### Step 3

### Methyl 1-amino-4-oxocyclohexanecarboxylate

Methyl 4-oxo-1-({[(phenylmethyl)oxy]carbonyl}amino)-2-cyclohexene-1-carboxylate (3 g, 9.9 mmol) was dissolved in CH₂Cl₂ (30 mL) and 0.5 g of 10% Pd/C was added. A H₂ atmosphere was established and the reaction was stirred overnight, filtered through celite, concentrated, and redissolved in CH₂Cl₂. 10% Pd/C (0.5 g) was added and an H₂ atmosphere was established and stirred overnight. The reaction was then filtered through celite and concentrated to afford 1.62 g of product as an oil.

### Step 4

### Methyl 1-{[(3-amino-2-naphthalenyl)carbonyl]amino}-4-oxocyclohexanecarboxylate

3-Amino-2-napthoic acid (155 mg, 0.69 mmol) was dissolved in DMF (4 mL) and diisopropylethylamine (0.20 g, 1.58 mmol) and HATU (0.26 g, 0.69 mmol) were added and stirred 20 min. Methyl 1-amino-4-oxocyclohexanecarboxylate (108 mg, 0.63 mmol) was dissolved in DMF (1 mL) and the solution was added to the reaction and heated to 50° C. for 60 min, cooled, and stirred 3 d. The mixture was diluted with ethyl acetate, washed with water and brine, dried (MgSO₄) and concentrated onto SiO₂. Chromatography on SiO₂ eluting with ethyl acetate/hexanes provided 148 mg of product.

### Step 5

### Methyl 1-({[3-({[(2,6-dichlorophenyl)amino]carbonyl}amino)-2-naphthalenyl]carbonyl}amino)-4-oxocyclohexanecarboxylate

Methyl 1-{[(3-amino-2-naphthalenyl)carbonyl]amino}-4-oxocyclohexanecarboxylate (0.14 g, 0.41 mmol) was dissolved in pyridine (3 mL) and 2,6-dichlorophenylisocyanate (0.39 g, 2.0 mmol) was added. After 30 min an additional 1 mL of pyridine was added and the reaction was diluted with ethyl acetate and concentrated onto SiO₂. Chromatography on SiO₂ eluting with ethyl acetate/hexanes afforded 210 mg of product.

### Step 6

### 1-({[3-({[(2,6-dichlorophenyl)amino]carbonyl}amino)-2-naphthalenyl]carbonyl}amino)-4-oxocyclohexanecarboxylic acid

Methyl 1-({[3-({[(2,6-dichlorophenyl)amino]carbonyl}amino)-2-naphthalenyl]carbonyl}amino)-4-oxocyclohexanecarboxylate (0.2 g, 0.37 mmol) was dissolved in 1:1 THF/MeOH (2 mL) and 2 M LiOH (0.95 mL) was added and the reaction was heated to 50° C. for 90 min and cooled. The solution was acidified with 1 M HCl (1.9 mL) and extracted with ethyl acetate. The extracts were dried (MgSO₄), concentrated, redissolved in CH₂Cl₂, and reconcentrated to provide a solid. Trituration of the solid with CH₂Cl₂ provided 25 mg of product. ES MS m/z 515 (M+H).

### Example 293

### 4-Oxo-1-({[3-({[(2,4,6-trimethylphenyl)amino]carbonyl}amino)-2-naphthalenyl]carbonyl}amino)cyclohexanecarboxylic acid

### Step 1

### Methyl 4-oxo-1-({[3-({[(2,4,6-trimethylphenyl)amino]carbonyl}amino)-2-naphthalenyl]carbonyl}amino)cyclohexanecarboxylate

Methyl 1-{[(3-amino-2-naphthalenyl)carbonyl]amino}-4-oxocyclohexanecarboxylate (0.99 g, 2.9 mmol) was dissolved in pyridine (13 mL) and 2,4,6-trimethylphenylisocyanate (2.34 g, 14.5 mmol) was added. After 4 h, the reaction was concentrated onto SiO₂. Chromatography on SiO₂ eluting with MeOH/CH₂Cl₂ afforded 0.84 g of product.

### Step 2

### 4-Oxo-1-({[3-({[(2,4,6-trimethylphenyl)amino]carbonyl}amino)-2-naphthalenyl]carbonyl}amino)cyclohexanecarboxylic acid

Methyl 4-oxo-1-({[3-({[(2,4,6-trimethylphenyl)amino]carbonyl}amino)-2-naphthalenyl]carbonyl}amino)cyclohexanecarboxylate (0.5 g, 0.99 mmol) was dissolved in 1:1 THF/MeOH (6 mL) and 2 M LiOH (2.5 mL) was added and the reaction stirred overnight. The solution was acidified with 5 M HCl (1 mL) and extracted with ethyl acetate. The extracts were dried (Na₂SO₄) and concentrated to 0.49 g of solid. 40 mg of the solid was purified by reverse-phase HPLC to provide 11 mg of product. ES MS m/z 488 (M+H).

### Example 294 & 295

### cis and trans 4-[(phenylmethyl)amino]-1-({[3-({[(2,4,6-trimethylphenyl)amino]carbonyl}amino)-2-naphthalenyl]carbonyl}amino)cyclohexanecarboxylic acid

### Step 1

### 4-[(phenylmethyl)amino]-1-({[3-({[(2,4,6-trimethylphenyl)amino]carbonyl}amino)-2-naphthalenyl]carbonyl}amino)cyclohexanecarboxylic acid

4-Oxo-1-({[3-({[(2,4,6-trimethylphenyl)amino]carbonyl}amino)-2-naphthalenyl]carbonyl}amino)cyclohexanecarboxylic acid (53 mg, 0.10 mmol) was dissolved in MeOH (1 mL) and polymer bound cyanoborohydride (80 mg, 0.32 mmol) and benzylamine (26 mg, 0.23 mmol) were added. The reaction was stirred overnight, filtered, and the solution was purified by reverse-phase HPLC to afford 5 mg each of the cis and trans products. Compound 1: ES MS m/z 579 (M+H). Compound 2: ES MS m/z 579 (M+H).

### Example 296

### 4-(hydroxyimino)-1-({[3-({[(2,4,6-trimethylphenyl)amino]carbonyl}amino)-2-naphthalenyl]carbonyl}amino)cyclohexanecarboxylic acid

A solution of hydroxylamine hydrochloride (11 mg, 0.15 mmol) and K₂CO₃ (20 mg, 0.18 mmol) in water (0.5 mL) were cooled to 5° C. and a solution of 4-Oxo-1-({[3-({[(2,4,6-trimethylphenyl)amino]carbonyl}amino)-2-naphthalenyl]carbonyl}amino)cyclohexanecarboxylic acid (50 mg, 0.10 mmol) dissolved in MeOH (0.5 mL) was added. The solution was stirred 1 h, diluted with water, and extracted with ethyl acetate. The aqueous layer was acidified with 1 M HCl (0.18 mL) and extracted with ethyl acetate. The combined extracts were dried (Na₂SO₄) and concentrated. The residue was redissolved in MeOH and purified by revere-phase HPLC to afford 3 mg of product. ES MS m/z 503 (M+H)

### Example 297

### (2S)-cyclohexyl({[2-({[(2,4,6-trimethylphenyl)amino]carbonyl}amino)-3-quinolinyl]carbonyl}amino)ethanoic acid

### Step 1

### Ethyl 2-cyano-3-(2-nitrophenyl)-2-propenoate

2-Nitrobenzaldehyde (5 g, 33.1 mmol) and n-Hexyltrimethylammonium bromide (1.2 g, 33.1 mmol) were suspended in water (290 mL) and stirred for 24 h. The stirring was stopped and the reaction stood for an additional 24 h. The resulting solid ethyl 2-cyano-3-(2-nitrophenyl)-2-propenoate was filtered off and dried under vacuum.

### Step 2

### Ethyl 2-amino-3-quinolinecarboxylate

Titanium tetrachloride (2.2 mL, 20 mmol) was added slowly to a stirring suspension of zinc (2.6 g, 40 mmol) in THF. When the addition was complete, the solution was refluxed for 2 h and cooled to RT. A solution of ethyl 2-cyano-3-(2-nitrophenyl)-2-propenoate (2.46 g, 10 mmol) in THF (20 mL) was added dropwise to the reaction. After 90 min the reaction was concentrated and the residue was poured onto 10% potassium carbonate and extracted with chloroform. The chloroform layer was filtered through celite, dried (MgSO4) and concentrated. The solids were concentrated onto SiO2 and purified by silica gel chromatography eluting with ethyl acetate/hexanes to afford 0.33 g of ethyl 2-amino-3-quinolinecarboxylate.

### Step 3

### 2-Amino-3-quinolinecarboxylic acid

Ethyl 2-amino-3-quinolinecarboxylate (0.23 g, 1.0 mmol) was dissolved in 1:1 THF/MeOH (5 mL) and 1 M NaOH (5.3 mL) was added. The reaction was stirred for 90 mins and then 5 M HCl (1 mL) was added. A colorless solid precipitated out of solution and was collected. After drying under vacuum, 0.11 g of 2-amino-3-quinolinecarboxylic acid was obtained.

### Step 4

### Methyl(2S)-{[(2-amino-3-quinolinyl)carbonyl]amino}(cyclohexyl)ethanoate

2-Amino-3-quinolinecarboxylic acid (0.11 g, 0.58 mmol) was dissolved in DMF (5 mL) and diisopropylethylamine (0.13 mL, 0.70 mmol) was added followed by HATU (0.27 g, 0.70 mmol). The reaction was heated to 50° C. for 30 min, the heating was removed and methyl(2S)-amino(cyclohexyl)ethanoate hydrochloride (0.15 g, 0.70 mmol) was added. After ca. 1 h, the reaction was diluted with ethyl acetate and washed water. The organic layer was dried over MgSO₄ and concentrated onto SiO₂. Chromatography on SiO₂ eluting with ethyl acetate/hexanes provided a yellow oil, which was redissolved in methylene chloride, filtered and concentrated to provide methyl(2S)-{[(2-amino-3-quinolinyl)carbonyl]amino}(cyclohexyl)ethanoate (0.12 g) as a yellow oil.

### Step 5

### Methyl(2S)-cyclohexyl({[2-({[(2,4,6-trimethylphenyl)amino]carbonyl}amino)-3-quinolinyl]carbonyl}amino)ethanoate

Methyl(2S)-{[(2-amino-3-quinolinyl)carbonyl]amino}(cyclohexyl)ethanoate (50 mg, 0.14 mmol) was dissolved in DMF (2 mL) and triethylamine (30 mg, 0.29 mmol) was added followed by 2,4,6-trimethylphenyl isocyanate (26 mg, 0.16 mmol). The solution was heated to 75° C. for ca. 90 min and cooled. The reaction was diluted with water and a solid precipitated out of solution. The solid was collected and dried under vacuum to provide 44 mg of product.

### Step 6

### (2S)-Cyclohexyl({[2-({[(2,4,6-trimethylphenyl)amino]carbonyl}amino)-3-quinolinyl]carbonyl}amino)ethanoic acid

A solution of LiOH (10 mg, 0.43 mmol) in water (0.5 mL) was added to a suspension of methyl(2S)-cyclohexyl({[2-({[(2,4,6-trimethylphenyl)amino]carbonyl}amino)-3-quinolinyl]carbonyl}amino)ethanoate (44 mg, 0.087 mmol) in THF (1 mL) and MeOH (1 mL) and stirred for ca. 3 h. 1 M HCl (0.43 mL) was added and a tan solid formed, which was filtered off and dried under vacuum to afford 23 mg of (2S)-cyclohexyl({[2-({[(2,4,6-trimethylphenyl)amino]carbonyl}amino)-3-quinolinyl]carbonyl}amino)ethanoic acid. ES MS m/z 489 (M+H).

### Example 298

### (2S)-Cyclohexyl({[3-({[(2,6-dichlorophenyl)amino]carbonyl}amino)-2-quinolinyl]carbonyl}amino)ethanoic acid

### Step 1

### N-[2-cyano-1-(phenylcarbonyl)-1,2-dihydro-3-quinolinyl]benzamide

3-Aminoquinoline (7.56 g, 52.4 mmol) was dissolved in CH₂Cl₂ (100 mL) and a solution of KCN (10.2 g, 157 mmol) in water (40 mL) was added. Benzoyl chloride (14.7 g, 105 mmol) was added dropwise and the solution was stirred for 3 h. The layers were separated and the organic layer was washed with saturated NaHCO₃, dried (Na₂SO₄) and concentrated to a foam. The foam was redissolved in CH₂Cl₂ and triturated with hexanes. The resulting solid was collected to afford 14.2 g of product.

### Step 2

### 3-Amino-2-quinolinecarboxylic acid

A suspension of -[2-cyano-1-(phenylcarbonyl)-1,2-dihydro-3-quinolinyl]benzamide (5 g, 13.2 mmol) in AcOH (10 mL) and 48% HBr (5 mL) was heated to 100° C. for 5 min and cooled. Ice water (10 mL) was added and the solution was cooled in an ice bath for 15 min. The resulting solid was collected by filtration and dried under vacuum. The solid was suspended in EtOH (60 mL) and 5 M NaOH (115 mL) and heated to reflux for ca. 18 h. The solution was cooled and concentrated to −50 mL and extracted with CH₂Cl₂. The pH of the aqueous phase was adjusted to 4 and the aqueous layer was reextracted with CH₂Cl₂. The extracts were concentrated and the resulting solid was washed with ethyl acetate to provide 0.6 g of product.

### Step 3

### Methyl(2S)-{[(3-amino-2-quinolinyl)carbonyl]amino}(cyclohexyl)ethanoate

Methyl(2S)-amino(cyclohexyl)ethanoate hydrochloride (0.33 g, 1.59 mmol) and 3-amino-2-quinolinecarboxylic acid (0.25 g, 1.32 mmol) were dissolved in DMF (6 mL) and diisopropylethylamine (0.38 g, 2.92 mmol) and HATU (0.60 g, 1.59 mmol) were added. The reaction was stirred for ca. 18 h and diluted with ethyl acetate and washed with water. The organic layer was dried (MgSO₄) and concentrated onto SiO₂. Chromatography on SiO₂ eluting with ethyl acetate/hexanes provided 0.24 g of product.

### Step 4

### Methyl(2S)-cyclohexyl({[3-({[(2,6-dichlorophenyl)amino]carbonyl}amino)-2-quinolinyl]carbonyl}amino)ethanoate

Methyl(2S)-{[(3-amino-2-quinolinyl)carbonyl]amino}(cyclohexyl)ethanoate (50 mg, 0.15 mmol) was dissolved in DMF (1 mL) and triethylamine (29 mg, 0.29 mmol) and 2,6-dichlorophenyl isocyanate (33 mg, 0.17 mmol) were added. The reaction was heated to 70° C. for ca. 90 min and cooled. The solution was diluted with ethyl acetate and washed with water. The extracts were dried (MgSO₄) and concentrated on SiO₂. Chromatography on SiO₂ eluting with ethyl acetate/hexanes provided 61 mg of product as a yellow solid.

### Step 5

### (2S)-Cyclohexyl({[3-({[(2,6-dichlorophenyl)amino]carbonyl}amino)-2-quinolinyl]carbonyl}amino)ethanoic acid

Methyl(2S)-cyclohexyl({[3-({[(2,6-dichlorophenyl)amino]carbonyl}amino)-2-quinolinyl]carbonyl}amino)ethanoate (61 mg, 0.11 mmol) was dissolved in 1:1 THF/MeOH (2 mL) and 2 M LiOH (0.28 ml, 0.57 mmol) was added and the reaction was stirred ca. 18 h. The solution was diluted with water, acidified with 1 M HCl (0.66 mL), and extracted with ethyl acetate. The extracts were dried (MgSO₄) and concentrated to afford 60 mg of product as a yellow foam. ES MS m/z 515 (M+H).

### Example 299

### (2S)-Cyclohexyl({[3-({[(2,4,6-trichlorophenyl)amino]carbonyl}amino)-2-quinolinyl]carbonyl}amino)ethanoic acid

### Step 1

### Methyl(2S)-cyclohexyl({[3-({[(2,4,6-trichlorophenyl)amino]carbonyl}amino)-2-quinolinyl]carbonyl}amino)ethanoate

Methyl(2S)-{[(3-amino-2-quinolinyl)carbonyl]amino}(cyclohexyl)ethanoate (50 mg, 0.15 mmol) was dissolved in DMF (1 mL) and triethylamine (30 mg, 0.29 mmol) and 2,4,6-trichlorophenyl isocyanate (39 mg, 0.17 mmol) were added. The reaction was heated to 70° C. for ca. 3 h, cooled and stirred overnight. The solution was diluted with water and extracted with ethyl acetate. The extracts were dried (MgSO₄) and concentrated on SiO2. Chromatography on SiO₂ eluting with ethyl acetate/hexanes provided 50 mg of product as a yellow solid.

### Step 2

### (2S)-Cyclohexyl({[3-({[(2,4,6-trichlorophenyl)amino]carbonyl}amino)-2-quinolinyl]carbonyl}amino)ethanoic acid

Methyl(2S)-cyclohexyl({[3-({[(2,4,6-trichlorophenyl)amino]carbonyl}amino)-2-quinolinyl]carbonyl}amino)ethanoate (50 mg, 0.088 mmol) was dissolved in 1:1 THF/MeOH (3 mL) and 2 M LiOH (0.44 mmol, 0.88 mmol) was added and the reaction was stirred 2 h. The solution was diluted with water, acidified with 1 M HCl (0.88 mL), and extracted with ethyl acetate. The extracts were dried (MgSO₄) and concentrated to afford 30 mg of product. ES MS m/z 551 (M+H).

### Example 300

### (2S)-Cyclohexyl({[3-({[(2,4,6-trimethylphenyl)amino]carbonyl}amino)-2-quinolinyl]carbonyl}amino)ethanoic acid

### Step 1

### Methyl(2S)-cyclohexyl({[3-({[(2,4,6-trimethylphenyl)amino]carbonyl}amino)-2-quinolinyl]carbonyl}amino)ethanoate

Methyl(2S)-{[(3-amino-2-quinolinyl)carbonyl]amino}(cyclohexyl)ethanoate (0.17 g, 0.50 mmol) was dissolved in pyridine (4 mL) and 2,4,6-trimethylphenyl isocyanate (0.41 g, 2.5 mmol) was added. The reaction was stirred for 3 h and then filtered, diluted with ethyl acetate, and washed with 1 M HCl. The extracts were dried (MgSO4), concentrated onto SiO2, and purified by chromatography on SiO2 eluting with ethyl acetate/hexanes to afford 0.21 g of product.

### Step 2

### (2S)-Cyclohexyl({[3-({[(2,4,6-trimethylphenyl)amino]carbonyl}amino)-2-quinolinyl]carbonyl}amino)ethanoic acid

Methyl(2S)-cyclohexyl({[3-({[(2,4,6-trimethylphenyl)amino]carbonyl}amino)-2-quinolinyl]carbonyl}amino)ethanoate (0.21 g, 0.41 mmol) was dissolved in 1:1 THF/MeOH (3 mL) and 2 M LiOH (1.0 mL) was added. The reaction was heated to 40° C. for 6 h, cooled, acidified with 5 M HCl (0.41 mL) and extracted with ethyl acetate. The extracts were dried (MgSO₄) and concentrated and the residue was redissolved in CH₂Cl₂. The organics were concentrated onto SiO₂ and purified by chromatography on SiO₂ eluting with ethyl acetate/hexanes to afford 130 mg of product. ES MS m/z 489 (M+H).

### Example 301

### 1-({[3-({[(2,4,6-Trichlorophenyl)amino]carbonyl}amino)-2-naphthalenyl]carbonyl}amino)cycloheptanecarboxylic acid

### Step 1

### Methyl 1-{[(3-amino-2-naphthalenyl)carbonyl]amino}cycloheptanecarboxylate

3-Amino-2-naphthoic acid (0.2 g, 1.0 mmol) and methyl 1-aminocycloheptanecarboxylate hydrochloride (0.25 g, 1.17 mmol) were dissolved in DMF (10 mL) and diisopropylethylamine (0.41 g, 3.20 mmol) and HATU (0.45 g, 1.17 mmol) were added. The solution was heated to 50° C. for 1 h and stirred overnight. The reaction was diluted with water and extracted with ethyl acetate. The extracts were washed with brine, dried (MgSO₄) and concentrated onto SiO₂. Chromatography on SiO₂ eluting with ethyl acetate/hexanes provided 0.29 g of product as a yellow solid.

### Step 2

### Methyl 1-({[3-({[(2,4,6-trichlorophenyl)amino]carbonyl}amino)-2-naphthalenyl]carbonyl}amino)cycloheptanecarboxylate

Methyl 1-{[(3-amino-2-naphthalenyl)carbonyl]amino}cycloheptanecarboxylate (0.1 g, 0.29 mmol) was dissolved in DMF (1 mL) and triethylamine (59 mg, 0.58 mmol) and 2,4,6-trichlorophenyl isocyanate (78 mg, 0.35 mmol) were added. The reaction was heated to 70° C. for 2 h and cooled. The reaction was diluted with water and extracted with ethyl acetate. The extracts were dried (MgSO₄) and concentrated onto SiO₂. Chromatography on SiO₂ eluting with ethyl acetate/hexanes provided 100 mg of product.

### Step 3

### 1-({[3-({[(2,4,6-Trichlorophenyl)amino]carbonyl}amino)-2-naphthalenyl]carbonyl}amino)cycloheptanecarboxylic acid

Methyl 1-({[3-({[(2,4,6-trichlorophenyl)amino]carbonyl}amino)-2-naphthalenyl]carbonyl}amino)cycloheptanecarboxylate (85 mg, 0.15 mmol) was dissolved in 1:1 THF/MeOH (2 mL) and 1 M NaOH (0.76 mL) was added. The solution was heated to 60° C. for 2 h and cooled. The reaction was stirred at RT for 15 h and then 0.8 mL of 1 M NaOH was added and heated to 60° C. for 4 h and cooled. The reaction was diluted with water, acidified with 1 M HCl (1.7 mL), and extracted with ethyl acetate. The extracts were dried (MgSO₄) and concentrated to afford 70 mg of product as a solid. ES MS m/z 549 (M+H).

### Example 302

### 1-({[3-({[(2,4,6-trimethylphenyl)amino]carbonyl}amino)-2-naphthalenyl]carbonyl}amino)cycloheptanecarboxylic acid

### Step 1

### Methyl 1-({[3-({[(2,4,6-trimethylphenyl)amino]carbonyl}amino)-2-naphthalenyl]carbonyl}amino)cycloheptanecarboxylate

Methyl 1-{[(3-amino-2-naphthalenyl)carbonyl]amino}cycloheptanecarboxylate (40 mg, 0.12 mmol) was dissolved in pyridine (2 mL) and 2,4,6-trimethylphenyl isocyanate (95 mg, 0.58 mmol) was added. The solution was stirred overnight and then concentrated onto SiO₂. Chromatography on SiO₂ eluting with ethyl acetate/hexanes provided 55 mg of product as an oil.

### Step 2

### Methyl 1-({[3-({[(2,4,6-trimethylphenyl)amino]carbonyl}amino)-2-naphthalenyl]carbonyl}amino)cycloheptanecarboxylate

Methyl 1-({[3-({[(2,4,6-trimethylphenyl)amino]carbonyl}amino)-2-naphthalenyl]carbonyl}amino)cycloheptanecarboxylate (55 mg, 0.11 mmol) was dissolved in 1:1 THF/MeOH (2 mL) and 1 M NaOH (1.1 mL) was added. The solution was heated to 60° C. for 2 h and cooled. The reaction was acidified with 1 M HCl (1.1 mL), and a solid precipitate formed. The solid was collected and dried under vacuum to provide 31 mg of product. ES MS m/z 488 (M+H).

### Example 303

### 1-({[3-({[(2,4,6-trichlorophenyl)amino]carbonyl}amino)-2-naphthalenyl]carbonyl}amino)cyclooctanecarboxylic acid

### Step 1

### Methyl 1-{[(3-amino-2-naphthalenyl)carbonyl]amino}cyclooctanecarboxylate

3-Amino-2-naphthoic acid (0.35 g, 1.58 mmol) and methyl 1-aminocyclooctanecarboxylate hydrochloride (0.32 g, 1.74 mmol) were dissolved in DMF (10 mL) and diisopropylethylamine (0.62 g, 4.76 mmol) and HATU (0.66 g, 1.74 mmol) were added. The solution was heated to 50° C. for 1 h and stirred overnight. The reaction was diluted with water and extracted with ethyl acetate. The extracts were washed with brine, dried (MgSO₄) and concentrated onto SiO₂. Chromatography on SiO₂ eluting with ethyl acetate/hexanes provided 0.29 g of product as a yellow solid.

### Step 2

### Methyl 1-({[3-({[(2,4,6-trichlorophenyl)amino]carbonyl}amino)-2-naphthalenyl]carbonyl}amino)cyclooctanecarboxylate

Methyl 1-{[(3-amino-2-naphthalenyl)carbonyl]amino}cyclooctanecarboxylate (40 mg, 0.11 mmol) was dissolved in pyridine (2 mL) and 2,4,6-trichlorophenylisocyanate (125 mg, 0.56 mmol) was added. The reaction was stirred for ca. 15 h and concentrated onto SiO2. Chromatography on SiO2 eluting with ethyl acetate/hexanes provided 65 mg of product.

### Step 3

### 1-({[3-({[(2,4,6-trichlorophenyl)amino]carbonyl}amino)-2-naphthalenyl]carbonyl}amino)cyclooctanecarboxylic acid

Methyl 1-({[3-({[(2,4,6-trichlorophenyl)amino]carbonyl}amino)-2-naphthalenyl]carbonyl}amino)cyclooctanecarboxylate (65 mg, 0.11 mmol) was dissolved in 1:1 THF/MeOH (2 mL) and 1 M NaOH (1.1 mL) was added. The reaction was heated to 90° C. for 6 h, cooled to RT and stirred overnight. 1 M HCl (1.2 mL) was added and the solution was extracted with ethyl acetate. The extracts were concentrated and the residue was dissolved in MeOH and purified by reverse phase HPLC to afford 22 mg of product. ES MS m/z 563 (M+H)

### Example 304

### 1-({[3-({[(2,4,6-trimethylphenyl)amino]carbonyl}amino)-2-naphthalenyl]carbonyl}amino)cyclooctanecarboxylic acid

### Step 1

### Methyl 1-({[3-({[(2,4,6-trimethylphenyl)amino]carbonyl}amino)-2-naphthalenyl]carbonyl}amino)cyclooctanecarboxylate

Methyl 1-{[(3-amino-2-naphthalenyl)carbonyl]amino}cyclooctanecarboxylate (40 mg, 0.11 mmol) was dissolved in pyridine (2 mL) and 2,4,6-trimethylphenylisocyanate (91 mg, 0.56 mmol) was added. The reaction was stirred for ca. 15 h and concentrated onto SiO₂. Chromatography on SiO₂ eluting with ethyl acetate/hexanes provided 58 mg of product.

### Step 2

### 1-({[3-({[(2,4,6-trimethylphenyl)amino]carbonyl}amino)-2-naphthalenyl]carbonyl}amino)cyclooctanecarboxylic acid

(65 mg, 0.11 mmol) was dissolved in 1:1 THF/MeOH (2 mL) and 1 M NaOH (1.1 mL) was added. The reaction was heated to 90° C. for 6 h, cooled to RT and stirred overnight. 1 M HCl (1.2 mL) was added and a solid precipitate formed. The solids were collected and dried under vacuum to provide 28 mg of product. ES MS m/z 502 (M+H)

### Example 305

### 1-({[3-({[(2,4,6-trimethylphenyl)amino]carbonyl}amino)-2-naphthalenyl]carbonyl}amino)cyclodecanecarboxylic acid

### Step 1

### Methyl 1-{[(3-amino-2-naphthalenyl)carbonyl]amino}cyclodecanecarboxylate

3-Amino-2-naphthoic acid (0.34 g, 1.54 mmol) was dissolved in DMF (10 mL) and diisopropylethylamine (0.45 g, 3.51 mmol) and HATU (0.59 g, 1.54 mmol) were added. The reaction was stirred for 20 min and methyl 1-aminocyclodecanecarboxylate hydrochloride (0.30 g, 1.40 mmol) was added. The solution was heated to 55° C. for 2 h, cooled, and diluted with ethyl acetate. The mixture was washed with water and the organics were dried (MgSO₄) and concentrated onto SiO₂. Chromatography on SiO₂ eluting with ethyl acetate/hexanes provided 0.34 g of product as a yellow solid.

### Step 2

### Methyl 1-({[3-({[(2,4,6-trimethylphenyl)amino]carbonyl}amino)-2-naphthalenyl]carbonyl}amino)cyclodecanecarboxylate

Methyl 1-{[(3-amino-2-naphthalenyl)carbonyl]amino}cyclodecanecarboxylate (0.34 g, 0.89 mmol) was dissolved in pyridine (6 mL) and 2,4,6-trimethylphenylisocyanate (0.72 g, 4.44 mmol) was added. The reaction was stirred for ca. 15 h and diluted with ethyl acetate. The solution was filtered and the filtrate was washed with 1 M HCl, dried (MgSO₄), and concentrated onto SiO₂. Chromatography on SiO₂ eluting with MeOH/CH₂Cl₂ provided a brown solid that was triturated with ethyl acetate to provide 0.28 g of product.

### Step 3

### 1-({[3-({[(2,4,6-trimethylphenyl)amino]carbonyl}amino)-2-naphthalenyl]carbonyl}amino)cyclodecanecarboxylic acid

Methyl 1-({[3-({[(2,4,6-trimethylphenyl)amino]carbonyl}amino)-2-naphthalenyl]carbonyl}amino)cyclodecanecarboxylate (0.28 g, 0.51 mmol) was suspended in 1:1 THF/MeOH and 2 M LiOH (1.3 mL) was added. The reaction was heated at 65° C. for 4 days, cooled, and acidified with 1 M HCl (2.6 mL). The solution was extracted with ethyl acetate and the organic layer was concentrated. The residue was suspended in ethyl acetate and concentrated onto SiO₂. Chromatography on SiO₂ eluting with ethyl acetate/hexanes provided 22 mg of product as a beige solid. ES MS m/z 530 (M+H).

### Example 306

### 1-({[3-({[(2,4,6-trimethylphenyl)amino]carbonyl}amino)-2-quinolinyl]carbonyl}amino)cycloheptanecarboxylic acid

### Step 1

### Methyl 1-{[(3-amino-2-quinolinyl)carbonyl]amino}cycloheptanecarboxylate

3-Amino-2-quinolinecarboxylic acid (0.25 g, 1.32 mmol) was dissolved in DMF (5 mL) and diisopropylethylamine (0.51 g, 3.98 mmol) and HATU (0.55 g, 1.46 mmol) were added. The reaction was stirred for 30 min and methyl 1-aminocycloheptanecarboxylate hydrochloride (0.30 g, 1.46 mmol) was added. The reaction was heated to 50° C. for 90 min and cooled. The reaction was diluted with ethyl acetate and washed with saturated NaHCO₃ solution and brine, dried (MgSO₄) and concentrated onto SiO₂. Chromatography on SiO₂ eluting with ethyl acetate/hexanes provided 0.21 g of product.

### Step 2

### Methyl 1-({[3-({[(2,4,6-trimethylphenyl)amino]carbonyl}amino)-2-quinolinyl]carbonyl}amino)cycloheptanecarboxylate

Methyl 1-{[(3-amino-2-quinolinyl)carbonyl]amino}cycloheptanecarboxylate (0.21 g, 0.61 mmol) was dissolved in pyridine (4 mL) and 2,4,6-trimethylphenylisocyanate (0.49 g, 3.07 mmol) was added. The reaction was stirred for 6 h, diluted with ethyl acetate and filtered. The filtrate was washed with 1 M HCl, dried (MgSO₄) and concentrated onto SiO₂. Chromatography on SiO₂ eluting with ethyl acetate/hexanes provided 198 mg of product.

### Step 3

### 1-({[3-({[(2,4,6-trimethylphenyl)amino]carbonyl}amino)-2-quinolinyl]carbonyl}amino)cycloheptanecarboxylic acid

Methyl 1-({[3-({[(2,4,6-trimethylphenyl)amino]carbonyl}amino)-2-quinolinyl]carbonyl}amino)cycloheptanecarboxylate (190 mg, 0.38 mmol) was dissolved in 1:1 THF/MeOH (2 mL) and 2 M LiOH (1.9 mL) was added. The reaction was heated to 55° C. for 4 h, cooled to RT and acidified with 5 M HCl (0.76 mL). The solution was extracted with ethyl acetate, dried (MgSO₄) and concentrated. The residue was redissolved in CH₂Cl₂ and concentrated onto SiO₂. Chromatography on SiO₂ eluting with ethyl acetate/hexanes provided 140 mg of product. ES MS m/z 489 (M+H)

### Example 307

### 1-({[3-({[(2,4,6-trimethylphenyl)amino]carbonyl}amino)-2-quinolinyl]carbonyl}amino)cyclooctanecarboxylic acid

### Step 1

### Methyl 1-{[(3-amino-2-quinolinyl)carbonyl]amino}cyclooctanecarboxylate

3-Amino-2-quinolinecarboxylic acid (0.14 g, 0.74 mmol) and methyl 1-aminocyclooctanecarboxylate hydrochloride (0.15 g, 0.81 mmol) were dissolved in DMF (5 mL) and diisopropylethylamine (0.34 g, 2.6 mmol) and HATU (0.31 g, 0.81 mmol) were added. The reaction was stirred for 3 h and diluted with ethyl acetate. The mixture was washed with water, dried (MgSO₄) and concentrated onto SiO₂. Chromatography on SiO₂ eluting with ethyl acetate/hexanes provided 0.19 g of product.

### Step 2

### Methyl 1-({[3-({[(2,4,6-trimethylphenyl)amino]carbonyl}amino)-2-quinolinyl]carbonyl}amino)cyclooctanecarboxylate

Methyl 1-{[(3-amino-2-quinolinyl)carbonyl]amino}cyclooctanecarboxylate (0.18 g, 0.50 mmol) was dissolved in pyridine (4 mL) and 2,4,6-trimethylphenylisocyanate (0.40 g, 2.5 mmol) was added. The reaction was stirred for 5 h, filtered, and the solids were washed with ethyl acetate. The filtrate was washed with 1 M HCl, dried (MgSO₄) and concentrated onto SiO₂. Chromatography on SiO₂ eluting with ethyl acetate/hexanes provided 0.25 g of product.

### Step 3

### 1-({[3-({[(2,4,6-trimethylphenyl)amino]carbonyl}amino)-2-quinolinyl]carbonyl}amino)cyclooctanecarboxylic acid

Methyl 1-({[3-({[(2,4,6-trimethylphenyl)amino]carbonyl}amino)-2-quinolinyl]carbonyl}amino)cyclooctanecarboxylate (230 mg, 0.44 mmol) was dissolved in 1:1 THF/MeOH (3.5 mL) and 2 M LiOH (2.2 mL) was added. The reaction was heated to 55° C. for 3 h, cooled to RT, diluted with water and acidified with 5 M HCl (0.89 mL). The solution was extracted with diethyl ether, dried (MgSO₄) and concentrated. The residue was triturated with diethyl ether to afford a solid, which was dried under vacuum to afford 197 mg of product. ES MS m/z 503 (M+H)

### Example 308

### 1-({[3-({[(4-bromo-2,6-dimethylphenyl)amino]carbonyl}amino)-2-naphthalenyl]carbonyl}amino)cycloheptanecarboxylic acid

### Step 1

### Methyl 1-({[3-({[(4-bromo-2,6-dimethylphenyl)amino]carbonyl}amino)-2-naphthalenyl]carbonyl}amino)cycloheptanecarboxylate

Methyl 1-{[(3-amino-2-naphthalenyl)carbonyl]amino}cycloheptanecarboxylate (0.31 g, 0.91 mmol) was dissolved in pyridine (7 mL) and 4-bromo-2,6-dimethylphenyl isocyanate (0.51 g, 2.27 mmol) was added. The solution was stirred overnight and then diluted with ethyl acetate, washed with 1 M HCl, dried (MgSO₄), and concentrated onto SiO₂. Chromatography on SiO₂ eluting with ethyl acetate/hexanes provided 0.48 g of product as a solid.

### Step 2

### 1-({[3-({[(4-bromo-2,6-dimethylphenyl)amino]carbonyl}amino)-2-naphthalenyl]carbonyl}amino)cycloheptanecarboxylic acid

Methyl 1-({[3-({[(4-bromo-2,6-dimethylphenyl)amino]carbonyl}amino)-2-naphthalenyl]carbonyl}amino)cycloheptanecarboxylate (83 mg, 0.14 mmol) was dissolved in 1:1 THF/MeOH (2 mL) and 2 M LiOH (0.73 mL) was added. The reaction was heated to 60° C. for 3 h, cooled to RT, acidified with 1 M HCl (1.46 mL) and extracted with ethyl acetate. The extracts were dried (MgSO₄) and concentrated. The residue was triturated with methanol to afford a solid, which was dried under vacuum to afford 58 mg of product. ES MS m/z 553 (M+H)

### Example 309

### 1-({[3-({[(2,6-dimethyl-4-propylphenyl)amino]carbonyl}amino)-2-naphthalenyl]carbonyl}amino)cycloheptanecarboxylic acid

### Step 1

### Methyl 1-[({3-[({[2,6-dimethyl-4-(2-propen-1-yl)phenyl]amino}carbonyl)amino]-2-naphthalenyl}carbonyl)amino]cycloheptanecarboxylate

Methyl 1-({[3-({[(4-bromo-2,6-dimethylphenyl)amino]carbonyl}amino)-2-naphthalenyl]carbonyl}amino)cycloheptanecarboxylate (0.2 g, 0.35 mmol) was suspended in CH₃CN (5 mL) and Pd(PPh₃)₄ (20 mg, 0.018 mmol) and allyltributylstannane (0.13 g, 0.38 mmol) were added. The reaction was purged with N₂ and heated to 150° C. for 30 min. The solution was cooled and concentrated onto SiO₂. Chromatography on SiO₂ eluting with ethyl acetate/hexanes provided 149 mg of product.

### Step 2

### Methyl 1-({[3-({[(2,6-dimethyl-4-propylphenyl)amino]carbonyl}amino)-2-naphthalenyl]carbonyl}amino)cycloheptanecarboxylate

Methyl 1-[({3-[({[2,6-dimethyl-4-(2-propen-1-yl)phenyl]amino}carbonyl)amino]-2-naphthalenyl}carbonyl)amino]cycloheptanecarboxylate (0.14 g, 0.26 mmol) was dissolved in ethyl acetate (5 mL) and 10% Pd/C (20 mg) was added. A H₂ atmosphere was established and the reaction was stirred overnight. The mixture was filtered through celite and washed with MeOH. The filtrate was concentrated onto SiO₂ and purified by chromatography on SiO₂ eluting with ethyl acetate/hexanes to afford 118 mg of product.

### Step 3

### 1-({[3-({[(2,6-dimethyl-4-propylphenyl)amino]carbonyl}amino)-2-naphthalenyl]carbonyl}amino)cycloheptanecarboxylic acid

Methyl 1-({[3-({[(2,6-dimethyl-4-propylphenyl)amino]carbonyl}amino)-2-naphthalenyl]carbonyl}amino)cycloheptanecarboxylate (118 mg, 0.22 mmol) was dissolved in 1:1 THF/MeOH (2 mL) and 2 M LiOH (1.1 mL) was added. The reaction was heated to 60° C. for 3 h, cooled to RT, acidified with 1 M HCl (2.2 mL) and extracted with ethyl acetate. The extracts were dried (MgSO₄) and concentrated. The residue was purified by chromatography on SiO₂ eluting with ethyl acetate/hexanes to provide 20 mg of product. ES MS m/z 516 (M+H)

### Example 310

### 2-({[3-({[(2,4,6-trimethylphenyl)amino]carbonyl}amino)-2-naphthalenyl]carbonyl}amino)-2,3-dihydro-1H-indene-2-carboxylic acid

### Step 1

### Methyl 2-({[(1,1-dimethylethyl)oxy]carbonyl}amino)-2,3-dihydro-1H-indene-2-carboxylate

2-({[(1,1-dimethylethyl)oxy]carbonyl}amino)-2,3-dihydro-1H-indene-2-carboxylic acid (0.26 g, 0.93 mmol) was dissolved in MeOH (6 mL) and a solution of trimethylsilyidiazomethane (2.5 mL) was added dropwise until a yellow color persisted. The solution was then concentrated to provide product as a solid which was used without further purification.

### Step 2

### Methyl 2-amino-2,3-dihydro-1H-indene-2-carboxylate trifluoroacetate

Methyl 2-({[(1 μl-dimethylethyl)oxy]carbonyl}amino)-2,3-dihydro-1H-indene-2-carboxylate (0.27 g, 0.93 mmol) was dissolved in CH₂Cl₂ (5 mL) and TFA (0.5 mL) was added and stirred overnight. The solution was concentrated to provide product as the TFA salt.

### Step 3

### Methyl 2-{[(3-amino-2-naphthalenyl)carbonyl]amino}-2,3-dihydro-1H-indene-2-carboxylate

3-Amino-2-quinolinecarboxylic acid (0.22 g, 1.0 mmol) was dissolved in DMF (6 mL) and diisopropylethylamine (0.41 g, 3.2 mmol) and HATU (0.38 g, 1.0 mmol) were added and stirred 20 min. Methyl 2-amino-2,3-dihydro-1H-indene-2-carboxylate trifluoroacetate (0.28 g, 0.92 mmol) was added and the reaction was heated to 55° C. for 1 h and cooled. The mixture was diluted with ethyl acetate, washed with water and brine, dried (Na₂SO₄), and concentrated onto SiO₂. Chromatography on SiO₂ eluting with ethyl acetate/hexanes provided 0.30 g of product.

### Step 4

### Methyl 2-({[3-({[(2,4,6-trimethylphenyl)amino]carbonyl}amino)-2-naphthalenyl]carbonyl}amino)-2,3-dihydro-1H-indene-2-carboxylate

Methyl 2-{[(3-amino-2-naphthalenyl)carbonyl]amino}-2,3-dihydro-1H-indene-2-carboxylate (0.30 g, 0.83 mmol) was dissolved in pyridine (5 mL) and 2,4,6-trimethylphenylisocyanate (0.67 g, 4.1 mmol) was added and stirred overnight. The solution was diluted with ethyl acetate, washed with 1 M HCl, filtered, dried (Na₂SO₄) and concentrated onto SiO₂. Chromatography on SiO₂ eluting with ethyl acetate/hexanes afforded 0.35 g of product.

### Step 5

### 2-({[3-({[(2,4,6-Trimethylphenyl)amino]carbonyl}amino)-2-naphthalenyl]carbonyl}amino)-2,3-dihydro-1H-indene-2-carboxylic acid

Methyl 2-({[3-({[(2,4,6-trimethylphenyl)amino]carbonyl}amino)-2-naphthalenyl]carbonyl}amino)-2,3-dihydro-1H-indene-2-carboxylate (0.35 g, 0.67 mmol) was dissolved in 1:1 THF/MeOH (3 mL) and 2 M LiOH (1.7 mL) was added. The reaction was heated to 55° C. for 2 h, cooled to RT and stirred overnight. The mixture was acidified with 5 M HCl (0.7 mL) and a solid formed. The solid was collected and dried under vacuum to provide 0.24 g of product. ES MS m/z 508 (M+H).

### Example 311

### 2-({[3-({[(2,4,6-trimethylphenyl)amino]carbonyl}amino)-2-naphthalenyl]carbonyl}amino)-1,2,3,4-tetrahydro-2-naphthalenecarboxylic acid

### Step 1

### Methyl 2-({[(1,1-dimethylethyl)oxy]carbonyl}amino)-1,2,3,4-tetrahydro-2-naphthalenecarboxylate

2-({[(1,1-Dimethylethyl)oxy]carbonyl}amino)-1,2,3,4-tetrahydro-2-naphthalenecarboxylic acid (1 g, 3.43 mmol) was dissolved in MeOH (30 mL) and a solution of trimethylsilyldiazomethane was added dropwise until a yellow color persisted and stirred 30 min. The solution was then concentrated to provide product as a solid which was used without further purification.

### Step 2

### Methyl 2-amino-1,2,3,4-tetrahydro-2-naphthalenecarboxylate trifluoroacetate

Methyl 2-({[(1,1-dimethylethyl)oxy]carbonyl}amino)-1,2,3,4-tetrahydro-2-naphthalenecarboxylate (I g, 3.4 mmol) was dissolved in CH₂Cl₂ and TFA (2 mL) was added and stirred overnight. The solution was concentrated and dried under vacuum to provide product as the TFA salt.

### Step 3

### Methyl 2-{[(3-amino-2-naphthalenyl)carbonyl]amino}-1,2,3,4-tetrahydro-2-naphthalenecarboxylate

3-Amino-2-quinolinecarboxylic acid (0.22 g, 0.96 mmol) and methyl 2-amino-1,2,3,4-tetrahydro-2-naphthalenecarboxylate trifluoroacetate (0.28 g, 0.88 mmol) were dissolved in DMF (5 mL) and diisopropylethylamine (0.40 g, 3.0 mmol) and HATU (0.37 g, 0.96 mmol) were added. The reaction was heated to 50° C. overnight and cooled. The mixture was diluted with ethyl acetate, washed with water, dried (MgSO₄) and concentrated onto SiO₂. Chromatography on SiO₂ eluting with ethyl acetate/hexanes provided 0.19 g of product.

### Step 4

### Methyl 2-({[3-({[(2,4,6-trimethylphenyl)amino]carbonyl}amino)-2-naphthalenyl]carbonyl}amino)-1,2,3,4-tetrahydro-2-naphthalenecarboxylate

Methyl 2-{[(3-amino-2-naphthalenyl)carbonyl]amino}-1,2,3,4-tetrahydro-2-naphthalenecarboxylate (0.19 g, 0.50 mmol) was dissolved in pyridine (5 mL) and 2,4,6-trimethylphenylisocyanate (0.42 g, 2.5 mmol) was added and stirred overnight. The solution was diluted with ethyl acetate, filtered, and concentrated onto SiO₂. Chromatography on SiO₂ eluting with ethyl acetate/hexanes afforded 0.24 g of product.

### Step 5

### 2-({[3-({[(2,4,6-trimethylphenyl)amino]carbonyl}amino)-2-naphthalenyl]carbonyl}amino)-1,2,3,4-tetrahydro-2-naphthalenecarboxylic acid

Methyl 2-({[3-({[(2,4,6-trimethylphenyl)amino]carbonyl}amino)-2-naphthalenyl]carbonyl}amino)-1,2,3,4-tetrahydro-2-naphthalenecarboxylate (0.24 g, 0.45 mmol) was dissolved in 1:1 THF/MeOH (4 mL) and 2 M LiOH (2.2 mL) was added. The reaction was heated to 55° C. for 3 h, cooled to RT, acidified with 1 M HCl (4.4 mL) and extracted with ethyl acetate. The extracts were dried (MgSO₄) and concentrated. The residue was purified by reverse-phase HPLC to provide 136 mg of product. ES MS m/z 522 (M+H)

### Example 312

### 2-Cyclohexyl-N-{[3-({[(2,4,6-trimethylphenyl)amino]carbonyl}amino)-2-quinolinyl]carbonyl}-D-alanine

### Step 1

### Methyl N-[(3-amino-2-quinolinyl)carbonyl]-2-cyclohexyl-D-alaninate

3-Amino-2-quinolinecarboxylic acid (0.25 g, 1.32 mmol) was dissolved in DMF (6 mL) and diisopropylethylamine (0.60 g, 4.64 mmol) and HATU (0.55 g, 1.46 mmol) were added. The reaction was stirred for 20 min and methyl 2-cyclohexyl-D-alaninate hydrochloride (0.32 g, 1.46 mmol) was added. The reaction was heated to 55° C. for 60 min and cooled. The reaction was diluted with ethyl acetate and washed with water and brine, dried (MgSO₄) and concentrated onto SiO₂. Chromatography on SiO₂ eluting with ethyl acetate/hexanes provided 0.32 g of product.

### Step 2

### Methyl 2-cyclohexyl-N-{[3-({[(2,4,6-trimethylphenyl)amino]carbonyl}amino)-2-quinolinyl]carbonyl}-D-alaninate

Methyl N-[(3-amino-2-quinolinyl)carbonyl]-2-cyclohexyl-D-alaninate (0.32 g, 0.90 mmol) was dissolved in pyridine (2 mL) and 2,4,6-trimethylphenylisocyanate (0.72 g, 4.5 mmol) was added. The reaction was stirred overnight, diluted with ethyl acetate, washed with 1 M HCl, and filtered. The filtrate was concentrated onto SiO₂ and purified by chromatography on SiO₂ eluting with ethyl acetate/hexanes provided 0.28 g of product.

### Step 3

### 2-Cyclohexyl-N-{[3-({[(2,4,6-trimethylphenyl)amino]carbonyl}amino)-2-quinolinyl]carbonyl}-D-alanine

Methyl 2-cyclohexyl-N-{[3-({[(2,4,6-trimethylphenyl)amino]carbonyl}amino)-2-quinolinyl]carbonyl}-D-alaninate (0.28 g, 0.54 mmol) was dissolved in 1:1 THF/MeOH (4 mL) and 2 M LiOH (2.7 mL) was added. The reaction was heated to 50° C. for 1 h, cooled to RT, acidified with 5 M HCl (1 mL) and extracted with ethyl acetate. The extracts were dried (Na₂SO₄) and concentrated onto SiO₂. Chromatography on SiO₂ eluting with ethyl acetate/hexanes afforded 130 mg of product. ES MS m/z 503 (M+H).

### Example 313

### N-{[3-({[(2,4,6-trimethylphenyl)amino]carbonyl}amino)-2-quinolinyl]carbonyl}-L-norleucine

### Step 1

### Methyl N-[(3-amino-2-quinolinyl)carbonyl]-L-norleucinate

3-Amino-2-quinolinecarboxylic acid (0.12 g, 0.66 mmol) was dissolved in DMF (6 mL) and diisopropylethylamine (0.26 g, 1.99 mmol) and HATU (0.28 g, 0.73 mmol) were added. The reaction was stirred for 20 min and methyl L-norleucinate hydrochloride (0.13 g, 0.73 mmol) was added and stirred for 3 days. The reaction was diluted with water and extracted with ethyl acetate. The extracts were dried (MgSO₄) and concentrated onto SiO₂. Chromatography on SiO₂ eluting with ethyl acetate/hexanes provided 0.11 g of product.

### Step 2

### Methyl N-{[3-({[(2,4,6-trimethylphenyl)amino]carbonyl}amino)-2-quinolinyl]carbonyl}-L-norleucinate

Methyl N-[(3-amino-2-quinolinyl)carbonyl]-L-norleucinate (50 mg, 0.16 mmol) was dissolved in pyridine (3 mL) and 2,4,6-trimethylphenylisocyanate (0.13 g, 0.79 mmol) was added. The reaction was stirred overnight, diluted with ethyl acetate and concentrated onto SiO₂. Chromatography on SiO₂ eluting with ethyl acetate/hexanes provided 46 mg of product.

### Step 3

### N-{[3-({[(2,4,6-trimethylphenyl)amino]carbonyl}amino)-2-quinolinyl]carbonyl}-L-norleucine

Methyl N-{[3-({[(2,4,6-trimethylphenyl)amino]carbonyl}amino)-2-quinolinyl]carbonyl}-L-norleucinate (46 mg, 0.096 mmol) was dissolved in 1:1 THF/MeOH (1 mL) and 2 M LiOH (0.48 mL) was added. After 5 min an additional 1 mL of MeOH was added and the reaction was stirred overnight. The reaction was acidified with 1 M HCl (1 mL) and a precipitate formed. The solid was collected and dried to provide 27 mg of product. ES MS m/z 463 (M+H).

### Example 314

### N-{[3-({[(2,6-dichlorophenyl)amino]carbonyl}amino)-2-quinolinyl]carbonyl}-L-norleucine

### Step 1

### Methyl N-{[3-({[(2,6-dichlorophenyl)amino]carbonyl}amino)-2-quinolinyl]carbonyl}-L-norleucinate

Methyl N-[(3-amino-2-quinolinyl)carbonyl]-L-norleucinate (56 mg, 0.18 mmol) was dissolved in pyridine (3 mL) and 2,6-dichlorophenylisocyanate (0.17 g, 0.88 mmol) was added. The reaction was stirred overnight, diluted with ethyl acetate and concentrated onto SiO₂. Chromatography on SiO₂ eluting with ethyl acetate/hexanes provided 90 mg of product.

### Step 2

### N-{[3-({[(2,6-dichlorophenyl)amino]carbonyl}amino)-2-quinolinyl]carbonyl}-L-norleucine

Methyl N-{[3-({[(2,6-dichlorophenyl)amino]carbonyl}amino)-2-quinolinyl]carbonyl}-L-norleucinate (90 mg, 0.18 mmol) was dissolved in 1:1 THF/MeOH (1 mL) and 2 M LiOH (0.93 mL) was added and the reaction was stirred overnight. The reaction was acidified with 1 M HCl (1.86 mL) and a precipitate formed. The solid was collected and dried to provide 74 mg of product. ES MS m/z 489 (M+H).

### Example 315

### 2-Propyl-N-{[3-({[(2,4,6-trimethylphenyl)amino]carbonyl}amino)-2-naphthalenyl]carbonyl}norvaline

### Step 1

### Methyl N-[(3-amino-2-naphthalenyl)carbonyl]-2-propylnorvalinate

3-Amino-2-napthoic acid (0.27 g, 1.44 mmol) was dissolved in DMF (5 mL) and diisopropylethylamine (0.56 g, 4.32 mmol) and HATU (0.60 g, 1.58 mmol) were added and stirred 15 min. Methyl 2-propylnorvalinate hydrochloride (0.27 g, 1.58 mmol) was added and the reaction was stirred overnight. The mixture was diluted with water and extracted with ethyl acetate. The extracts were dried (MgSO₄) and concentrated onto SiO₂. Chromatography on SiO₂ eluting with ethyl acetate/hexanes provided 0.28 g of product.

### Step 2

### Methyl 2-propyl-N-{[3-({[(2,4,6-trimethylphenyl)amino]carbonyl}amino)-2-naphthalenyl]carbonyl}norvalinate

Methyl N-[(3-amino-2-naphthalenyl)carbonyl]-2-propylnorvalinate (53 mg, 0.15 mmol) was dissolved in DMF (2 mL) and triethylamine (31 mg, 0.30 mmol) and 2,4,6-trimethylphenyl isocyanate (41 mg, 0.25 mmol) were added. The reaction was heated to 70° C. for 3 h and then stirred at RT overnight. The reaction was filtered and the filtrate was diluted with ethyl acetate, washed with water, dried (MgSO₄), and concentrated onto SiO₂. Chromatography on SiO₂ eluting with ethyl acetate/hexanes afforded 37 mg of product.

### Step 3

### 2-Propyl-N-{[3-({[(2,4,6-trimethylphenyl)amino]carbonyl}amino)-2-naphthalenyl]carbonyl}norvaline

Methyl 2-propyl-N-{[3-({[(2,4,6-trimethylphenyl)amino]carbonyl}amino)-2-naphthalenyl]carbonyl}norvalinate (37 mg, 0.073 mmol) was dissolved in 1:1 THF/MeOH (1 mL) and 2 M LiOH (0.53 mL) was added and the reaction was heated to 60° C. overnight. The reaction was cooled, diluted with water, acidified with 1 M HCl (1.86 mL) and extracted with ethyl acetate. The extracts were concentrated and the residue was dissolved in MeOH (1 mL) and purified by reverse-phase HPLC to afford 27 mg of product. ES MS m/z 490 (M+H).

### Example 316

### N-{[3-({[(2,6-dichlorophenyl)amino]carbonyl}amino)-2-naphthalenyl]carbonyl}-2-propylnorvaline

### Step 1

### Methyl N-{[3-({[(2,6-dichlorophenyl)amino]carbonyl}amino)-2-naphthalenyl]carbonyl}-2-propylnorvalinate

Methyl N-[(3-amino-2-naphthalenyl)carbonyl]-2-propylnorvalinate (53 mg, 0.15 mmol) was dissolved in DMF (2 mL) and triethylamine (31 mg, 0.30 mmol) and 2,6-dichlorophenyl isocyanate (35 mg, 0.18 mmol) were added. The reaction was heated to 70° C. for 3 h and then stirred at RT overnight. The reaction was diluted with ethyl acetate, washed with water, dried (MgSO₄), and concentrated onto SiO₂. Chromatography on SiO₂ eluting with ethyl acetate/hexanes afforded 60 mg of product.

### Step 2

### N-{[3-({[(2,6-dichlorophenyl)amino]carbonyl}amino)-2-naphthalenyl]carbonyl}-2-propylnorvaline

Methyl N-{[3-({[(2,6-dichlorophenyl)amino]carbonyl}amino)-2-naphthalenyl]carbonyl}-2-propylnorvalinate (60 mg, 0.11 mmol) was dissolved in 1:1 THF/MeOH (1 mL) and 2 M LiOH (0.35 mL) was added and the reaction was heated to 60° C. overnight. The reaction was cooled, diluted with water, acidified with 1 M HCl (1.86 mL) and extracted with ethyl acetate. The extracts were concentrated and the residue was dissolved in MeOH (1 mL) and purified by reverse-phase HPLC to afford 15 mg of product. ES MS m/z 516 (M+H).

### Example 317

### (2S)-({[5-chloro-3-({[(2,4,6-trimethylphenyl)amino]carbonyl}amino)-2-pyridinyl]carbonyl}amino)(cyclohexyl)ethanoic acid

### Step 1

### 2-Bromo-5-chloro-3-nitropyridine according to the literature

2-Amino-5-chloro-3-nitropyridine (25.5 g, 147 mmol) was added to a solution of 48% HBr (83 mL) at 0° C. Bromine (25.1 mL) was added dropwise to the solution, maintaining the reaction temperature below 0° C. A solution of NaNO₂ (35.3 g, 511 mmol) in water (48 mL) was added, again maintaining the reaction temperature below 0° C. After the addition was complete, the reaction was stirred 45 min and then a solution of NaOH (53.8 g) in water (80 mL) was added, maintaining the reaction temperature below 20° C. The mixture was stirred an additional 1 h and the resulting product was filtered off and dried to afford 26 g of product.

### Step 2

### 5-Chloro-3-nitro-2-pyridinecarbonitrile

2-Bromo-5-chloro-3-nitropyridine (1.5 g, 6.31 mmol) and CuCN (1.13 g, 12.63 mmol) were dissolved in NMP (12 mL) and heated to 170° C. for 10 min and cooled. The solution was poured onto water and ethyl acetate was added. The mixture was filtered through celite and the organic layer was separated, washed with brine, dried (MgSO₄) and concentrated onto SiO₂. Chromatography on SiO₂ eluting with ethyl acetate/hexanes afforded 0.97 go of product. This reaction was repeated to provide additional product.

### Step 3

### 3-Amino-5-chloro-2-pyridinecarboxamide

5-Chloro-3-nitro-2-pyridinecarbonitrile (0.97 g, 5.28) was dissolved in EtOH (20 mL) and Raney-nickel (100 mg, prewashed with water, 5% AcOH, water and EtOH) was added. The mixture was placed under 50 psi H₂ and shaken for 3 h. The mixture was then filtered through celite and concentrated to afford 0.76 g of product as a brown solid. This reaction was repeated to provide additional product.

### Step 4

### 3-Amino-5-chloro-2-pyridinecarboxylic acid

3-Amino-5-chloro-2-pyridinecarboxamide (2.5 g, 14.5 mmol) was suspended in concentrated HCl (25 mL) and heated to reflux for 15 h and cooled in an ice bath. Precipitated solid was filtered off to provide 1.0 g of the product as the hydrochloride salt and the pH of the filtrate was adjusted to 6 with 5 M NaOH and extracted with ethyl acetate. The extracts were concentrated to afford 1.27 g of the product.

### Step 5

### Methyl(2S)-{[(3-amino-5-chloro-2-pyridinyl)carbonyl]amino}(cyclohexyl)ethanoate

3-Amino-5-chloro-2-pyridinecarboxylic acid hydrochloride (0.21 g, 1.0 mmol) was dissolved in DMF (5 mL) and diisopropylethylamine (0.52 g, 4.01 mmol) and HATU (0.42 g, 1.10 mmol) were added and stirred 20 min. Methyl(2S)-amino(cyclohexyl)ethanoate hydrochloride (0.23 g, 1.10 mmol) was added and the reaction was stirred for 20 min and then diluted with water and extracted with ethyl acetate. The extracts were dried (MgSO₄) and concentrated onto SiO₂. Chromatography on SiO2 eluting with ethyl acetate/hexanes provided 0.28 g of product.

### Step 6

### Methyl(2S)-({[5-chloro-3-({[(2,4,6-trimethylphenyl)amino]carbonyl}amino)-2-pyridinyl]carbonyl}amino)(cyclohexyl)ethanoate

Methyl(2S)-{[(3-amino-5-chloro-2-pyridinyl)carbonyl]amino}(cyclohexyl)ethanoate (0.28 g, 0.86 mmol) was dissolved in pyridine (5 mL) and 2,4,6-trimethylphenylisocyanate (0.69 g, 4.29 mmol) was added. The reaction was stirred overnight, diluted with ethyl acetate and concentrated onto SiO₂. Chromatography on SiO₂ eluting with ethyl acetate/hexanes provided product contaminated with an impurity. The mixture was repurified by reverse-phase HPLC to afford 176 mg of product as a colorless foam.

### Step 7

### (2S)-({[5-chloro-3-({[(2,4,6-trimethylphenyl)amino]carbonyl}amino)-2-pyridinyl]carbonyl}amino)(cyclohexyl)ethanoic acid

Methyl(2S)-({[5-chloro-3-({[(2,4,6-trimethylphenyl)amino]carbonyl}amino)-2-pyridinyl]carbonyl}amino)(cyclohexyl)ethanoate (60 mg, 0.1 mmol) was dissolved in 1:1 THF/MeOH (1 mL) and 2 M LiOH (0.5 mL) was added and the reaction was stirred 5 min and a solid precipitate formed. The reaction was acidified with 1 M HCl (1.0 mL) and a colorless solid resulted. The solid was collected and dried under vacuum to afford 45 mg of product. ES MS m/z 473 (M+H).

### Example 318

### N-{[5-chloro-3-({[(2,4,6-trimethylphenyl)amino]carbonyl}amino)-2-pyridinyl]carbonyl}-O-(1,1-dimethylethyl)-L-threonine

### Step 1

### Methyl N-[(3-amino-5-chloro-2-pyridinyl)carbonyl]-O-(1,1-dimethylethyl)-L-threoninate

3-Amino-5-chloro-2-pyridinecarboxylic acid (0.22 g, 1.27 mmol) and methyl O-(1,1-dimethylethyl)-L-threoninate (0.35 g, 1.52 mmol) were dissolved in DMF (4 mL) and diisopropylethylamine (0.58 g, 4.46 mmol) and HATU (0.58 g, 1.52 mmol) were added and stirred 3 days. The reaction was diluted with ethyl acetate and washed with water. The organic layer was dried (MgSO₄) and concentrated onto SiO₂. Chromatography on SiO₂ eluting with ethyl acetate/hexanes provided 0.28 g of product.

### Step 2

### Methyl N-{[5-chloro-3-({[(2,4,6-trimethylphenyl)amino]carbonyl}amino)-2-pyridinyl]carbonyl}-O-(1,1-dimethylethyl)-L-threoninate

Methyl N-[(3-amino-5-chloro-2-pyridinyl)carbonyl]-O-(1,1-dimethylethyl)-L-threoninate hydrochloride (0.26 g, 0.75 mmol) was dissolved in pyridine (5 mL) and 2,4,6-trimethylphenylisocyanate (0.60 g, 3.78 mmol) was added. The reaction was stirred 5 h, diluted with ethyl acetate, washed with 1 M HCl, and concentrated onto SiO₂. Chromatography on SiO₂ eluting with ethyl acetate/hexanes provided 170 mg of product.

### Step 3

### N-{[5-chloro-3-({[(2,4,6-trimethylphenyl)amino]carbonyl}amino)-2-pyridinyl]carbonyl}-O-(1,1-dimethylethyl)-L-threonine

Methyl N-{[5-chloro-3-({[(2,4,6-trimethylphenyl)amino]carbonyl}amino)-2-pyridinyl]carbonyl}-O-(1,1-dimethylethyl)-L-threoninate (0.17 g, 0.33 mmol) was dissolved in 1:1 THF/MeOH (2 mL) and 2 M LiOH (0.84 mL) was added and the reaction was stirred 2 h and acidified with 5 M HCl (0.33 mL) and extracted with ethyl acetate. The extracts were dried (MgSO₄) and concentrated to provide 154 mg of product as a pale yellow foam. ES MS m/z 491 (M+H).

### Example 319

### 1-({[5-chloro-3-({[(2,4,6-trimethylphenyl)amino]carbonyl}amino)-2-pyridinyl]carbonyl}amino)cycloheptanecarboxylic acid

### Step 1

### Methyl 1-{[(3-amino-5-chloro-2-pyridinyl)carbonyl]amino}cycloheptanecarboxylate

3-Amino-5-chloro-2-pyridinecarboxylic acid (0.22 g, 1.27 mmol) was dissolved in DMF (10 mL) and diisopropylethylamine (0.55 g, 4.29 mmol) and HATU (0.51 g, 1.34 mmol) were added and stirred 30 min. Methyl 1-aminocycloheptanecarboxylate hydrochloride (0.28 g, 1.34 mmol) was added and the mixture was heated to 55° C. for 2 h and cooled. The reaction was diluted with ethyl acetate and washed with water and brine. The organic layer was dried (MgSO₄) and concentrated onto SiO₂. Chromatography on SiO₂ eluting with ethyl acetate/hexanes provided 0.55 g of product.

### Step 2

### Methyl 1-({[5-chloro-3-({[(2,4,6-trimethylphenyl)amino]carbonyl}amino)-2-pyridinyl]carbonyl}amino)cycloheptanecarboxylate

Methyl 1-{[(3-amino-5-chloro-2-pyridinyl)carbonyl]amino}cycloheptanecarboxylate (0.55 g, 1.69 mmol) was dissolved in pyridine (5 mL) and 2,4,6-trimethylphenylisocyanate (1.4 g, 8.44 mmol) was added. The reaction was stirred overnight, diluted with ethyl acetate, filtered, washed with 1 M HCl and brine, and concentrated onto SiO₂. Chromatography on SiO₂ eluting with ethyl acetate/hexanes provided 170 mg of product.

### Step 3

### 1-({[5-chloro-3-({[(2,4,6-trimethylphenyl)amino]carbonyl}amino)-2-pyridinyl]carbonyl}amino)cycloheptanecarboxylic acid

Methyl 1-({[5-chloro-3-({[(2,4,6-trimethylphenyl)amino]carbonyl}amino)-2-pyridinyl]carbonyl}amino)cycloheptanecarboxylate (0.17 g, 0.35 mmol) was dissolved in 1:1 THF/MeOH (2 mL) and 2 M LiOH (1.7 mL) was added. The reaction was heated to 55° C. for 3 h, cooled, acidified with 5 M HCl (0.7 mL) and extracted with ethyl acetate. The extracts were dried (MgSO₄) and concentrated to provide 130 mg of product. ES MS m/z 473 (M+H).

### Example 320

### 1-({[5-Chloro-3-({[(2,6-dimethyl-4-propylphenyl)amino]carbonyl}amino)-2-pyridinyl]carbonyl}amino)cyclooctanecarboxylic acid

### Step 1

### Methyl 1-{[(3-amino-5-chloro-2-pyridinyl)carbonyl]amino}cyclooctanecarboxylate

3-Amino-5-chloro-2-pyridinecarboxylic acid (0.53 g, 2.53 mmol) and methyl 1-aminocycloheptanecarboxylate (0.52 g, 2.78 mmol) were dissolved in DMF (10 mL) and diisopropylethylamine (1.14 g, 8.87 mmol) and HATU (1.06 g, 2.78 mmol) were added and stirred for 3 h. The mixture was diluted with ethyl acetate, washed with water, dried (MgSO₄), and concentrated onto SiO₂. Chromatography on SiO₂ eluting with ethyl acetate/hexanes provided 0.68 g of product.

### Step 2

### Methyl 1-({[3-({[(4-bromo-2,6-dimethylphenyl)amino]carbonyl}amino)-5-chloro-2-pyridinyl]carbonyl}amino)cyclooctanecarboxylate

Methyl 1-{[(3-amino-5-chloro-2-pyridinyl)carbonyl]amino}cyclooctanecarboxylate (0.2 g, 0.59 mmol) was dissolved in pyridine (5 mL) and 4-bromo-2,6-dimethylphenylisocyanate (0.27 g, 1.17 mmol) was added. The reaction was stirred 4 h, diluted with ethyl acetate, washed with 1 M HCl, dried (MgSO₄), and concentrated to a solid. The solid was triturated with MeOH to provide 0.27 g of product

### Step 3

### Methyl 1-[({5-chloro-3-[({[2,6-dimethyl-4-(2-propen-1-yl)phenyl]amino}carbonyl)amino]-2-pyridinyl}carbonyl)amino]cyclooctanecarboxylate

Methyl 1-({[3-({[(4-bromo-2,6-dimethylphenyl)amino]carbonyl}amino)-5-chloro-2-pyridinyl]carbonyl}amino)cyclooctanecarboxylate (143 mg, 0.25 mmol) was suspended in CH₃CN (5 mL) and Pd(PPh₃)₄ (15 mg, 0.012 mmol) and allyltributylstannane (0.10 g, 0.30 mmol) were added. The reaction was purged with N₂ and heated to 150° C. for 20 min. The solution was cooled and concentrated onto SiO₂. Chromatography on SiO₂ eluting with ethyl acetate/hexanes provided 100 mg of product.

### Step 4

### Methyl 1-({[5-chloro-3-({[(2,6-dimethyl-4-propylphenyl)amino]carbonyl}amino)-2-pyridinyl]carbonyl}amino)cyclooctanecarboxylate

Methyl 1-[({5-chloro-3-[({[2,6-dimethyl-4-(2-propen-1-yl)phenyl]amino}carbonyl)amino]-2-pyridinyl}carbonyl)amino]cyclooctanecarboxylate (100 mg, 0.19 mmol) was dissolved in ethyl acetate (3 mL) and 10% Pd/C (20 mg) was added. A H₂ atmosphere was established and the reaction was stirred overnight. The mixture was filtered through celite and concentrated to afford 64 mg of product.

### Step 5

### 1-({[5-Chloro-3-({[(2,6-dimethyl-4-propylphenyl)amino]carbonyl}amino)-2-pyridinyl]carbonyl}amino)cyclooctanecarboxylic acid

Methyl 1-({[5-chloro-3-({[(2,6-dimethyl-4-propylphenyl)amino]carbonyl}amino)-2-pyridinyl]carbonyl}amino)cyclooctanecarboxylate (0.64 mg, 0.12 mmol) was dissolved in 1:1 THF/MeOH (2 mL) and 2 M LiOH (0.6 mL) was added. The reaction was heated to 60° C. for 4 h, cooled, acidified with 1 M HCl (1.2 mL) and extracted with ethyl acetate. The extracts were dried (MgSO₄) and concentrated and the residue was redissolved in MeOH. After standing 2 days, a solid film resulted which was sonicated in MeOH (1 mL) to provide a colorless solid which was dried under vacuum to afford 18 mg of product. ES MS m/z 515 (M+H).

### Example 321

### (2S)-Cyclohexyl({[5-phenyl-3-({[(2,4,6-trimethylphenyl)amino]carbonyl}amino)-2-pyridinyl]carbonyl}amino)ethanoic acid

Methyl(2S)-({[5-chloro-3-({[(2,4,6-trimethylphenyl)amino]carbonyl}amino)-2-pyridinyl]carbonyl}amino)(cyclohexyl)ethanoate (48 mg, 0.08 mmol), phenyl boronic acid (11 mg, 0.09 mmol), and PdCl₂(PCy₃)₂ (3 mg, 0.004 mmol) were dissolved in CH₃CN (1.8 mL) and 2 M Na₂CO₃ (0.16 mL) was added. The mixture was heated to 150° C. for 10 min and cooled. 2 M LiOH (1.0 mL) was added and the mixture was stirred overnight. 5 M HCl (0.45 ml) was added and the mixture was stirred vigorously until a solid resulted, which was filtered off and dissolved in MeOH. Reverse-phase HPLC purification provided 29 mg of product as the TFA salt. ES MS m/z 515 (M+H).

### Example 322

### (2S)-Cyclohexyl({[5-[4-(methyloxy)phenyl]-3-({[(2,4,6-trimethylphenyl)amino]carbonyl}amino)-2-pyridinyl]carbonyl}amino)ethanoic acid

Methyl(2S)-({[5-chloro-3-({[(2,4,6-trimethylphenyl)amino]carbonyl}amino)-2-pyridinyl]carbonyl}amino)(cyclohexyl)ethanoate (48 mg, 0.08 mmol), 4-methoxyphenyl boronic acid (13 mg, 0.09 mmol), and PdCl₂(PCy₃)₂ (3 mg, 0.004 mmol) were dissolved in CH₃CN (1.8 mL) and 2 M Na₂CO₃ (0.16 mL) was added. The mixture was heated to 150° C. for 10 min and cooled. 2 M LiOH (1.0 mL) was added and the mixture was heated to 50° C. for 90 min and cooled. 5 M HCl (0.55 ml) was added and the mixture was stirred vigorously until a solid resulted, which was filtered off and triturated with MeOH to provide 20 mg of product. ES MS m/z 545 (M+H).

### Example 323

### O-(1,1-dimethylethyl)-N-{[5-[4-(methyloxy)phenyl]-3-({[(2,4,6-trimethylphenyl)amino]carbonyl}amino)-2-pyridinyl]carbonyl}-L-threonine

N-{[5-chloro-3-({[(2,4,6-trimethylphenyl)amino]carbonyl}amino)-2-pyridinyl]carbonyl}-O-(1,1-dimethylethyl)-L-threonine (50 mg, 0.10 mmol), 4-methoxyphenylboronic acid (19 mg, 0.12 mmol), and PdCl₂(PCy₃)₂ (4 mg, 0.005 mmol) were dissolved in CH₃CN (2.5 mL) and 2 M Na₂CO₃ (0.15 mL) was added. The reaction was heated to 160° C. for 15 min and cooled. The reaction was diluted with ethyl acetate and water and 1 M HCl (0.30 mL) was added. The organic layer was separated, dried (MgSO₄), and concentrated onto SiO₂. Chromatography on SiO₂ eluting with ethyl acetate/hexanes provided 7 mg of product. ES MS m/z 563 (M+H)

### Example 324

### N-{[5-(3,4-Difluorophenyl)-3-({[(2,4,6-trimethylphenyl)amino]carbonyl}amino)-2-pyridinyl]carbonyl}-O-(1,1-dimethylethyl)-L-threonine

N-{[5-chloro-3-({[(2,4,6-trimethylphenyl)amino]carbonyl}amino)-2-pyridinyl]carbonyl}-O-(1,1-dimethylethyl)-L-threonine (78 mg, 0.16 mmol), 3,4-difluorophenylboronic acid (30 mg, 0.19 mmol), and PdCl2(PCy3)2 (6 mg, 0.008 mmol) were dissolved in CH3CN (3 mL) and 2 M Na2CO3 (0.23 mL) was added. The reaction was heated to 160° C. for 10 min and cooled. The reaction was diluted with water, 1 M HCl (0.48 mL) was added, and the mixture was extracted with ethyl acetate. The extracts were dried (MgSO₄) and concentrated. The residue was redissolved in MeOH (1 mL) and purified by reverse-phase HPLC to afford 18 mg of product. ES MS m/z 569 (M+H).

### Example 325

### 1-({[5-[4-(methyloxy)phenyl]-3-({[(2,4,6-trimethylphenyl)amino]carbonyl}amino)-2-pyridinyl]carbonyl}amino)cycloheptanecarboxylic acid

1-({[5-chloro-3-({[(2,4,6-trimethylphenyl)amino]carbonyl}amino)-2-pyridinyl]carbonyl}amino)cycloheptanecarboxylic acid (0.11 g, 0.23 mmol), 4-methoxyphenylboronic acid (42 mg, 0.28 mmol), and PdCl₂(PCy₃)₂ (9 mg, 0.01 mmol) were dissolved in CH₃CN (4 mL) and 2 M Na₂CO₃ (0.46 mL) was added. The reaction was heated to 150° C. for 15 min and cooled. The reaction was acidified with 5 M HCl (0.18 mL) and extracted with ethyl acetate. The extracts were dried (MgSO₄) and concentrated onto SiO₂. Chromatography on SiO₂ eluting with ethyl acetate/hexanes provided a solid which was triturated with MeOH to afford 18 mg of product. ES MS m/z 545 (M+H).

### Example 326

### (2S)-({[6-Bromo-3-({[(2,4,6-trichlorophenyl)amino]carbonyl}amino)-1-benzofuran-2-yl]carbonyl}amino)(cyclohexyl)ethanoic acid

### Step 1

### 3-Amino-6-bromo-1-benzofuran-2-carboxylic acid (U22318-11)

Ethyl 3-amino-6-bromo-1-benzofuran-2-carboxylate (1.05 g, 5.11 mmol) was dissolved in 1:1 THF/MeOH (20 mL) and 2 M LiOH (5.1 mL) was added. The reaction was stirred overnight. The reaction was acidified with 1 M HCl (10 mL) and ethyl acetate was added. The organic layer was separated and concentrated to afford 1.0 g of product.

### Step 2

### Methyl(2S)-{[(3-amino-6-bromo-1-benzofuran-2-yl)carbonyl]amino}(cyclohexyl)ethanoate

3-Amino-6-bromo-1-benzofuran-2-carboxylic acid (0.5 g, 1.95 mmol) was dissolved in DMF (10 mL) and diisopropylethylamine (0.55 g, 4.19 mmol) and HATU (0.89 g, 2.34 mmol) were added and stirred for 15 min. Methyl(2S)-amino(cyclohexyl)ethanoate hydrochloride (0.49 g, 2.34 mmol) was added and stirred overnight. Water was added and the mixture was extracted with ethyl acetate. The extracts were dried (MgSO₄) and concentrated onto SiO₂. Chromatography on SiO₂ eluting with ethyl acetate/hexanes afforded 0.58 g of product.

### Step 3

### Methyl(2S)-({[6-bromo-3-({[(2,4,6-trichlorophenyl)amino]carbonyl}amino)-1-benzofuran-2-yl]carbonyl}amino)(cyclohexyl)ethanoate

Methyl(2S)-{[(3-amino-6-bromo-1-benzofuran-2-yl)carbonyl]amino}(cyclohexyl)ethanoate (50 mg, 0.12 mmol) was dissolved in pyridine (1 mL) and 2,4,6-trichlorophenylisocyanate (30 mg, 0.13 mmol) was added. The reaction was heated to 50° C. for 15 h and then an additional 60 mg of 2,4,6-trichlorophenylisocyanate was added and stirred for 15 min at 50° C. and then cooled and stirred for 24 h. The reaction mixture was then concentrated onto SiO₂ and purified by chromatography on SiO₂ eluting with ethyl acetate/hexanes provided 77 mg of product as a solid.

### Step 4

### (2S)-({[6-Bromo-3-({[(2,4,6-trichlorophenyl)amino]carbonyl}amino)-1-benzofuran-2-yl]carbonyl}amino)(cyclohexyl)ethanoic acid

Methyl(2S)-({[⁶-bromo-3-({[(2,4,6-trichlorophenyl)amino]carbonyl}amino)-1-benzofuran-2-yl]carbonyl}amino)(cyclohexyl)ethanoate (77 mg, 0.12 mmol) was dissolved in 1:1 THF/MeOH (5 mL) and 2 M LiOH (0.6 mL) was added. The reaction was stirred for 4 h, diluted with water, acidified with 1 M HCl (1.2 mL) and a solid formed. The solid was collected and dried under vacuum to provide 62 mg of product. MS m/z 618 (M+H).

### Example 327

### (2S)-({[6-Bromo-3-({[(2,6-dichlorophenyl)amino]carbonyl}amino)-1-benzofuran-2-yl]carbonyl}amino)(cyclohexyl)ethanoic acid

### Step 1

### Methyl(2S)-({[6-bromo-3-({[(2,6-dichlorophenyl)amino]carbonyl}amino)-1-benzofuran-2-yl]carbonyl}amino)(cyclohexyl)ethanoate

Methyl(2S)-{[(3-amino-6-bromo-1-benzofuran-2-yl)carbonyl]amino}(cyclohexyl)ethanoate (50 mg, 0.12 mmol) was dissolved in DMF (1 mL) and triethylamine (19 mg, 0.14 mmol) and 2,6-dichlorophenylisocyanate (28 mg, 0.14 mmol) was added. The reaction was heated to 60° C. for 4 h and stirred overnight. The reaction mixture was then diluted with water and extracted with ethyl acetate. The extracts were dried (MgSO₄) and concentrated onto SiO₂. Chromatography on SiO₂ eluting with ethyl acetate/hexanes provided 40 mg of product.

### Step 2

### (2S)-({[⁶-Bromo-3-({[(2,6-dichlorophenyl)amino]carbonyl}amino)-1-benzofuran-2-yl]carbonyl}amino)(cyclohexyl)ethanoic acid

Methyl(2S)-({[6-bromo-3-({[(2,6-dichlorophenyl)amino]carbonyl}amino)-1-benzofuran-2-yl]carbonyl}amino)(cyclohexyl)ethanoate (40 mg, 0.066 mmol) was dissolved in 1:1 THF/MeOH (2 mL) and 2 M LiOH (0.33 mL) was added. The reaction was stirred for 4 h, diluted with water, acidified with 1 M HCl (0.7 mL) and extracted with ethyl acetate. The extracts were dried (MgSO₄) and concentrated to a solid. The solid was triturated with warm MeOH to provide 9 mg of product. MS m/z 584 (M+H).

### Example 328

### O-(phenylmethyl)-N-{[3-({[(2,4,6-trimethylphenyl)amino]carbonyl}amino)-2-naphthalenyl]carbonyl}-L-threonine

### Step 1

### Methyl N-(triphenylmethyl)-L-threoninate

To a cooled (0° C.) solution of methyl L-threoninate hydrochloride (5.0 g, 29.48 mmol) and triethylamine (5.97 g, 58.97 mmol) in chloroform (100 ml) was added trityl chloride as a solid (8.22 g, 29.49 mmol). The reaction was stirred for 12 hours and allowed to come to RT. The reaction was concentrated in vacuo and then dissolved in ethyl acetate and washed with saturated sodium chloride, 10% citric acid, saturated NaHCO₃, and saturated sodium chloride. The organic layer was dried over MgSO₄, filtered and stripped to give 10.16 g of product as a fluffy cream solid.

### Step 2

### Methyl(2R,3S)-3-methyl-1-(triphenylmethyl)-2-aziridinecarboxylate

To a cooled (0° C.) solution of methyl N-(triphenylmethyl)-L-threoninate (10.16 g, 27.95 mmol) in anhydrous pyridine was added methanesulfonyl chloride (9.61 g, 83.85 mmol) and the reaction was allowed to stir for 12 hours and allowed to come to RT. The solvent was removed in vacuo and the residue dissolved in ethyl acetate. The organic layer was washed with saturated sodium chloride and then dried over MgSO₄, filtered and stripped to 12.33 g of amber oil which was then dissolved in 80 ml of anhydrous THF and to which was added triethylamine (8.50 g, 84.01 mmol) and heated to 80° C. and allowed to reflux for 48 hours. The heat was removed and the reaction was concentrated in vacuo and the residue dissolved in ethyl acetate and washed successively with saturated sodium chloride, 10% citric acid, saturated NaHCO₃ and saturated sodium chloride. The ethyl acetate layer was dried over MgSO₄, filtered and stripped to give 9.04 g of amber oil. Chromatography on silica gel with hexane/ethyl acetate gave 5.26 g of product fluffy cream solid.

### Step 3

### 2-methyl 1-(phenylmethyl) (2R,3S)-3-methyl-1,2-aziridinedicarboxylate

To a solution of methyl(2R,3S)-3-methyl-1-(triphenylmethyl)-2-aziridinecarboxylate (5.26 g, 14.72 mmol) in CHCl₃ (12 ml) and MeOH (12 ml) cooled to 0° C. was added 11.6 ml of TFA and allowed to stir at 0° C. for 2.5 hours. The reaction was then concentrated in vacuo and evaporated with ether newly added several times to remove TFA. The residue was dissolved in ether which was extracted with water three times. To the aqueous extract at 0° C. was added NaHCO₃ (5.84 g, 69.52 mmol), benzyl chloroformate (2.51 g, 14.71 mmol) and 50 ml of ethyl acetate under vigorous stirring for 1.5 hours. The ethyl acetate layer was separated and the water layer back-extracted. The organics were dried over MgSO₄, filtered and concentrated to give 2.96 g of light yellow oil. Chromatography on silica gel with hexane/ethyl acetate gave 2.45 g of product as a clear oil.

### Step 4

### Methyl O-(phenylmethyl)-N-{[(phenylmethyl)oxy]carbonyl}-L-allothreoninate

To a solution of 2-methyl 1-(phenylmethyl) (2R,3S)-3-methyl-1,2-aziridinedicarboxylate (0.5 g, 2.06 mmol) in CHCl₃ (10 ml) was added benzyl alcohol (2.16 g, 20.00 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 2.66 g of product as a clear oil.

### Step 5

### Methyl O-(phenylmethyl)-L-allothreoninate

Palladium (10% weight on activated carbon, catalytic amount) was added to a solution of methyl O-(phenylmethyl)-N-{[(phenylmethyl)oxy]carbonyl}-L-allothreoninate (2.66 g, 7.44 mmol) in 10 ml of EtOH in a flask under nitrogen. A balloon of H₂ was then affixed to the reaction flask and the reaction was stirred for 3 hours at RT. The reaction was then filtered through a filter paper and the solvent evaporated to give 1.96 g of clear oil.

### Step 6

### Methyl N-[(3-amino-2-naphthalenyl)carbonyl]-O-(phenylmethyl)-L-threoninate

HATU (0.76 g, 2.00 mmol) was added to a solution of 3-amino-2-naphthalenecarboxylic acid (0.31 g, 1.66 mmol), methyl O-(phenylmethyl)-L-allothreoninate (0.45 g, 2.01 mmol) and diisopropylethylamine (0.26 g, 2.01 mmol) in 10 mL of DMF. The mixture was stirred at RT for ca. 15 h. The reaction was diluted with ethyl acetate and washed with water. The organic layer was dried over magnesium sulfate, filtered, and the solvent evaporated. Chromatography on silica gel with hexane/ethyl acetate gave 0.476 g of yellow oil.

### Step 7

### Methyl O-(phenylmethyl)-N-{[3-({[(2,4,6-trimethylphenyl)amino]carbonyl}amino)-2-naphthalenyl]carbonyl}-L-threoninate

Methyl N-[(3-amino-2-naphthalenyl)carbonyl]-O-(phenylmethyl)-L-threoninate (0.47 g, 1.19 mmol) was dissolved in pyridine (10 mL) and 2,4,6-trimethylphenylisocyanate (0.58 g, 3.59 mmol) was added. The reaction was stirred 4 h, diluted with ethyl acetate, and washed with 1 M HCl. The extracts were dried and concentrated onto SiO₂. Chromatography on SiO₂ eluting with ethyl acetate/hexanes provided 0.59 g of product.

### Step 8

### O-(phenylmethyl)-N-{[3-({[(2,4,6-trimethylphenyl)amino]carbonyl}amino)-2-naphthalenyl]carbonyl}-L-threonine

Methyl O-(phenylmethyl)-N-{[3-({[(2,4,6-trimethylphenyl)amino]carbonyl}amino)-2-naphthalenyl]carbonyl}-L-threoninate (0.59 g, 1.06 mmol) was dissolved in 1:1 THF/MeOH (10 mL) and 2 M LiOH (5.3 mL) was added. The reaction was stirred for 3 h, acidified with 1 M HCl (10.6 mL) and extracted with ethyl acetate. The extracts were dried (MgSO₄) and concentrated onto SiO₂. Chromatography on SiO₂ eluting with ethyl acetate/hexanes afforded an impure solid. Purification of 200 mg of the solid by reverse-phase HPLC afforded 53 mg of product. MS m/z 539 (M+H).

### Example 329

### (3R)-3-[(phenylmethyl)oxy]-N-{[3-({[(2,4,6-trimethylphenyl)amino]carbonyl}amino)-2-naphthalenyl]carbonyl}-L-norvaline

### Step 1

### (1R)-1-[(2S,5R)-5-(1-methylethyl)-3,6-bis(methyloxy)-2,5-dihydro-2-pyrazinyl]-1-propanol

(2R)-2-(1-methylethyl)-3,6-bis(methyloxy)-2,5-dihydropyrazine (1 g, 5.42 mmol) was dissolved in THF (30 mL) and cooled to −78° C. A solution of n-BuLi (3.6 mL of a 1.6 M solution) was added dropwise and stirred for 30 min. Propionaldehyde (0.35 mL, 5.97 mmol) was added and the reaction was stirred for 4 h and poured onto water and Et₂O. The organic layer was separated, dried (MgSO₄), and concentrated onto SiO₂. Chromatography on SiO₂ eluting with ethyl acetate/hexanes afforded 0.85 g of product as a clear oil.

### Step 2

### (2R,5S)-2-(1-methylethyl)-3,6-bis(methyloxy)-5-{(1R)-1-[(phenylmethyl)oxy]propyl}-2,5-dihydropyrazine

(1R)-1-[(2S,5R)-5-(1-methylethyl)-3,6-bis(methyloxy)-2,5-dihydro-2-pyrazinyl]-1-propanol (0.8 g, 3.30 mmol) was dissolved in DMF (20 mL) and the solution was cooled to 0° C. Sodium hydride (0.15 g, 3.80 mmol) was added and stirred 30 min and then benzyl bromide (0.62 g, 3.63 mmol) was added and stirred overnight. The reaction was diluted with water, extracted with ethyl acetate, and the extracts were dried (MgSO₄) and concentrated onto SiO₂. Chromatography on SiO₂ eluting with ethyl acetate/hexanes afforded 0.51 g of product.

### Step 3

### Methyl(3R)-3-[(phenylmethyl)oxy]-L-norvalinate

(2R,5S)-2-(1-Methylethyl)-3,6-bis(methyloxy)-5-{(1R)-1-[(phenylmethyl)oxy]propyl}-2,5-dihydropyrazine (0.51 g, 1.53 mmol) was dissolved in CH₃CN (6 mL) and 0.5 N HCl (6.1 mL) was added and the solution was stirred for 4 days. Sodium chloride and Et₂O were added to the solution and the pH was adjusted to 9 with ammonium hydroxide. The mixture was extracted with Et₂O, the extracts were combined and concentrated to afford 0.49 g of oil as a 1:1 mixture of desired product and methyl D-valinate.

### Step 4

### Methyl(3R)—N-[(3-amino-2-naphthalenyl)carbonyl]-3-[(phenylmethyl)oxy]-L-norvalinate

A 1:1 mixture of methyl(3R)-3-[(phenylmethyl)oxy]-L-norvalinate and methyl D-valinate (0.49 g, 1.32 mmol) and 3-amino-2-naphthalenecarboxylic acid (0.35 g, 1.59 mmol) was dissolved in DMF (10 mL) and diisopropylethylamine (0.51 g, 3.98 mmol) was added followed by HATU (0.60 g, 1.59 mmol). The solution was stirred overnight and then diluted with water and extracted with ethyl acetate. The extracts were dried (MgSO₄), concentrated onto SiO₂, and purified by chromatography on SiO₂ eluting with ethyl acetate/hexanes to afford 0.48 g of a 1:1 mixture of product and methyl N-[(3-amino-2-naphthalenyl)carbonyl]-D-valinate.

### Step 5

### Methyl(3R)-3-[(phenylmethyl)oxy]-N-{[3-({[(2,4,6-trimethylphenyl)amino]carbonyl}amino)-2-naphthalenyl]carbonyl}-L-norvalinate

A 1:1 mixture of methyl(3R)—N-[(3-amino-2-naphthalenyl)carbonyl]-3-[(phenylmethyl)oxy]-L-norvalinate and methyl N-[(3-amino-2-naphthalenyl)carbonyl]-D-valinate (0.48 g, 0.68 mmol) was dissolved in pyridine (7 mL) and 2,4,6-trimethylphenylisocyanate (0.33 g, 2.03 mmol) was added and stirred for 3 h. The solution was diluted with ethyl acetate, washed with 1 M HCl, dried (MgSO₄) and concentrated onto SiO₂. Chromatography on SiO2 eluting with ethyl acetate/hexanes afforded 0.48 g of a 1:1 mixture of product and methyl N-{[3-({[(2,4,6-trimethylphenyl)amino]carbonyl}amino)-2-naphthalenyl]carbonyl}-D-valinate.

### Step 6

### (3R)-3-[(phenylmethyl)oxy]-N-{[3-({[(2,4,6-trimethylphenyl)amino]carbonyl}amino)-2-naphthalenyl]carbonyl}-L-norvaline

A 1:1 mixture of methyl(3R)-3-[(phenylmethyl)oxy]-N-{[3-({[(2,4,6-trimethylphenyl)amino]carbonyl}amino)-2-naphthalenyl]carbonyl}-L-norvalinate and methyl N-{[3-({[(2,4,6-trimethylphenyl)amino]carbonyl}amino)-2-naphthalenyl]carbonyl}-D-valinate (0.48 g, 0.46 mmol) was dissolved in 1:1 THF/MeOH (3 mL) and 2 M LiOH (2.3 mL) was added. The reaction was stirred for 3 h, acidified with 1 M HCl (4.6 mL) and extracted with ethyl acetate. The extracts were dried (MgSO₄) and concentrated. A 100 mg sample of the residue was dissolved in MeOH (1 mL) and purified by reverse-phase HPLC to afford 34 mg of product. MS m/z 554 (M+H).

### Example 330

### N-(cyclohexylmethyl)-3-({[(2,4,6-trimethylphenyl)amino]carbonyl}amino)-2-naphthalenecarboxamide

### Step 1

### 3-({[(2,4,6-trimethylphenyl)amino]carbonyl}amino)-2-naphthalenecarboxylic acid

3-amino-2-naphthalenecarboxylic acid (5.00 g, 26.71 mmol) in 100 mL of DMF was treated with triethylamine (5.40 g, 53.37 mmol) and 2-isocyanato-1,3,5-trimethylbenzene (4.7 g, 29.16 mmol) and was heated to 70° C. for ca. 3 hours. The reaction was quenched with 1N HCl and extracted with ethyl acetate. The organic layer was dried over magnesium sulfate, filtered, and the solvent evaporated to give 7.95 g (84%) of product.

### Step 2

### N-(cyclohexylmethyl)-3-({[(2,4,6-trimethylphenyl)amino]carbonyl}amino)-2-naphthalenecarboxamide

3-({[(2,4,6-Trimethylphenyl)amino]carbonyl}amino)-2-naphthalenecarboxylic acid (0.1 g, 0.29 mmol) and (cyclohexylmethyl)amine (36 mg, 0.31 mmol) were dissolved in DMF (1.5 mL) and diisopropylethylamine (74 mg, 0.1 mL) and HATU (0.12 g, 0.31 mmol) were added and stirred ca. 18 h. The solution was diluted with ethyl acetate and washed with water. The extracts were dried (MgSO₄), concentrated onto SiO₂, and purified by chromatography on SiO₂ to afford 34 mg of product. ES MS m/z 444 (M+H).

### Example 331

### N-{[3-({[(2,6-dichlorophenyl)amino]carbonyl}amino)-2-naphthalenyl]carbonyl}-N-(phenylmethyl)-beta-alanine

### Step 1

### Ethyl N-[(3-amino-2-naphthalenyl)carbonyl]-N-(phenylmethyl)-beta-alaninate

3-Amino-2-naphthoic acid (0.2 g, 1.0 mmol) and ethyl N-(phenylmethyl)-beta-alaninate (0.24 g, 1.17 mmol) were dissolved in DMF (5 mL) and HATU (0.44 g, 1.17 mmol) and diisopropylethylamine (0.27 g, 2.13 mmol) were added. The solution was stirred ca. 90 min, diluted with ethyl acetate and washed with water and brine. The organic layer was dried (MgSO₄), concentrated onto SiO₂, and purified by chromatography on SiO₂ eluting with ethyl acetate/hexanes to afford 0.34 g of product as a brown solid.

### Step 2

### Ethyl N-{[3-({[(2,6-dichlorophenyl)amino]carbonyl}amino)-2-naphthalenyl]carbonyl}-N-(phenylmethyl)-beta-alaninate

Ethyl N-[(3-amino-2-naphthalenyl)carbonyl]-N-(phenylmethyl)-beta-alaninate (0.11 g, 0.30 mmol) was dissolved in DMF (2 mL) and triethylamine (62 mg, 0.61 mmol) was added followed by 2,6-dichlorophenyl isocyanate (69 mg, 0.36 mmol). The reaction was heated to 70° C. for ca. 60 min and cooled. The solution was diluted with ethyl acetate, washed with water, and dried (MgSO₄). The extracts were concentrated onto SiO₂ and purified by chromatography on SiO₂ eluting with ethyl acetate/hexanes to afford 0.11 g of product as an oil.

### Step 3

### N-{[3-({[(2,6-dichlorophenyl)amino]carbonyl}amino)-2-naphthalenyl]carbonyl}-N-(phenylmethyl)-beta-alanine

Ethyl N-{[3-({[(2,6-dichlorophenyl)amino]carbonyl}amino)-2-naphthalenyl]carbonyl}-N-(phenylmethyl)-beta-alaninate (0.1 g, 0.17 mmol) was dissolved in 1:1 THF/MeOH (2 mL) and 1 M NaOH (0.88 mL) was added. The solution was heated to 50° C. for 90 min and cooled. The reaction was diluted with water, acidified with 1 M HCl (1.1 mL), and extracted with ethyl acetate. The extracts were dried (MgSO₄) and concentrated to afford 45 mg of product as a solid. ES MS m/z 536 (M+H).

### Example 332

### N-(phenylmethyl)-N-{[3-({[(2,4,6-trichlorophenyl)amino]carbonyl}amino)-2-naphthalenyl]carbonyl}glycine

### Step 1

### Ethyl N-[(3-amino-2-naphthalenyl)carbonyl]-N-(phenylmethyl)glycinate

3-Amino-2-naphthoic acid (0.2 g, 1.0 mmol) and ethyl N-(phenylmethyl)glycinate (0.23 g, 1.17 mmol) were dissolved in DMF (5 mL) and diisopropylamine (0.41 g, 3.20 mmol) and HATU (0.45 g, 1.17 mmol) were added. The reaction was heated to 50° C. for 1 hour and then stirred ca. 18 h at RT. The reaction was diluted with ethyl acetate and washed with water and brine. The extracts were dried (MgSO₄), concentrated onto SiO₂, and purified by chromatography on SiO₂ eluting with ethyl acetate/hexanes to afford 0.35 g of product as a gold oil.

### Step 2

### Ethyl N-{[3-({[(2,4,6-trichlorophenyl)amino]carbonyl}amino)-2-naphthalenyl]carbonyl}-N-(phenylmethyl)glycinate

Ethyl N-[(3-amino-2-naphthalenyl)carbonyl]-N-(phenylmethyl)glycinate (0.1 g, 0.27 mmol) was dissolved in DMF (4 mL) and triethylamine (56 mg, 0.55 mmol) and 2,4,6-trichlorophenyl isocyanate (74 mg, 0.33 mmol) were added. The reaction was heated to 70° C. for 2 h and cooled. The reaction was diluted with water and extracted with ethyl acetate. The extracts were dried (MgSO₄) and concentrated onto SiO₂. Chromatography on SiO₂ eluting with ethyl acetate/hexanes provided 90 mg of product.

### Step 3

### N-(phenylmethyl)-N-{[3-({[(2,4,6-trichlorophenyl)amino]carbonyl}amino)-2-naphthalenyl]carbonyl}glycine

Ethyl N-{[3-({[(2,4,6-trichlorophenyl)amino]carbonyl}amino)-2-naphthalenyl]carbonyl}-N-(phenylmethyl)glycinate (90 mg, 0.15 mmol) was dissolved in 1:1 THF/MeOH (2 mL) and 1 M NaOH (0.77 mL) was added. The solution was heated to 50° C. for 2 h and stirred an additional 18 h. The reaction was diluted with water, acidified with 1 M HCl (0.8 mL), and extracted with ethyl acetate. The extracts were dried (MgSO4) and concentrated. The residue was dissolved in MeOH (1 mL) and purified by reverse phase HPLC. The fractions were concentrated to afford 47 mg of product as a tan solid. ES MS m/z 577 (M+H).

### Example 333

### 1-{[3-({[(2,6-Dichlorophenyl)amino]carbonyl}amino)-2-naphthalenyl]carbonyl}-3-piperidinecarboxylic acid

### Step 1

### Ethyl 1-[(3-amino-2-naphthalenyl)carbonyl]-3-piperidinecarboxylate

3-Amino-2-naphthoic acid (0.25 g, 1.33 mmol) and ethyl nipecotate (0.22 g, 1.47 mmol) were dissolved in DMF (5 mL) and HATU (0.56 g, 1.47 mmol) and diisopropylethylamine (0.34 g, 2.67 mmol) were added. The solution was stirred 90 min, diluted with ethyl acetate, and washed with water and brine. The organic layer was dried (MgSO4) and concentrated onto SiO2. Chromatography on SiO2 eluting with ethyl acetate/hexanes provided 0.35 g of product as an oil.

### Step 2

### Ethyl 1-{[3-({[(2,6-dichlorophenyl)amino]carbonyl}amino)-2-naphthalenyl]carbonyl}-3-piperidinecarboxylate

Ethyl 1-[(3-amino-2-naphthalenyl)carbonyl]-3-piperidinecarboxylate (0.1 g, 0.30 mmol) was dissolved in DMF (2 mL) and triethylamine (62 mg, 0.61 mmol) and 2,6-dichlorophenyl isocyanate (69 mg, 0.36 mmol) were added. The reaction was heated to 70° C. for 1 h and cooled. The reaction was diluted with water and extracted with ethyl acetate. The extracts were dried (MgSO₄) and concentrated onto SiO₂. Chromatography on SiO₂ eluting with ethyl acetate/hexanes provided 38 mg of product.

### Step 3

### 1-{[3-({[(2,6-Dichlorophenyl)amino]carbonyl}amino)-2-naphthalenyl]carbonyl}-3-piperidinecarboxylic acid

Ethyl 1-{[3-({[(2,6-dichlorophenyl)amino]carbonyl}amino)-2-naphthalenyl]carbonyl}-3-piperidinecarboxylate (32 mg, 0.062 mmol) was dissolved in 1:1 THF/MeOH (1 mL) and 1 M NaOH (0.31 mL) was added. The solution was heated to 50° C. for 90 min and cooled. The reaction was diluted with water, acidified with 1 M HCl (0.4 mL), and extracted with ethyl acetate. The extracts were dried (MgSO4) and concentrated to afford 23 mg of product as a colorless solid. ES MS m/z 486 (M+H).

### Example 334

### 1-{[3-({[(2,6-dimethylphenyl)amino]carbonyl}amino)-2-naphthalenyl]carbonyl}-L-proline

### Step 1

### 1,1-dimethylethyl 1-{[3-({[(2,6-dimethylphenyl)amino]carbonyl}amino)-2-naphthalenyl]carbonyl}-L-prolinate

A solution of HATU (0.37 g, 0.97 mmol) and 3-({[(2,6-dimethylphenyl)amino]carbonyl}amino)-2-naphthalenecarboxylic acid (0.29 g, 0.88 mmol) in DMF (5 mL) were stirred at RT for 5 minutes then 1,1-dimethylethyl L-prolinate (0.18 g, 1.06 mmol) was added. After 3 h, ethyl acetate and 1N HCl were added. The organic layer was washed with 1N HCl, water, brine solution, dried over MgSO₄, filtered and concentrated. The crude product was purified on silica gel using an ISCO chromatography system (gradient: 100% hexanes to 100% ethyl acetate over 25 minutes) to give 0.28 g (65%) of desired product.

### Step 2

### 1-{[3-({[(2,6-dimethylphenyl)amino]carbonyl}amino)-2-naphthalenyl]carbonyl}-L-proline

TFA (3 mL) was added to a solution of 1,1-dimethylethyl 1-{[3-({[(2,6-dimethylphenyl)amino]carbonyl}amino)-2-naphthalenyl]carbonyl}-L-prolinate (0.30 g, 0.62 mmol) in DCM (10 mL). The solution was stirred at RT with occasional heating until all the starting material was consumed as evident from TLC. The solution was concentrated to dryness and DCM (5 mL) and MeOH (1 mL) were added, followed by Et₂O (20 mL) and hexanes (5 mL). The resulting precipitate was filtered and dried under vacuum to give 0.026 (10%) of the title product as a white solid. ES MS m/z 430 (M−H).

### Example 335

### 3-methyl-N-{[3-({[(2,4,6-trimethylphenyl)amino]carbonyl}amino)-2-naphthalenyl]carbonyl}-L-valine

### Step 1

### N-[(3-amino-2-naphthalenyl)carbonyl]-3-methyl-L-valine

HATU (0.88 g, 2.33 mmol) was added to a mixture of 3-amino-2-naphthalenecarboxylic acid (0.36 g, 1.94 mmol), methyl 3-methyl-L-valinate hydrochloride (0.40 g, 2.14 mmol) and N,N-diisopropylethylamine (0.68 mL, 3.88 mmol) in DMF (10 mL). The solution was stirred at RT for 16 h then saturated NaHCO₃ solution and ethyl acetate were added. The organic layer was washed with water, brine, dried over MgSO₄, filtered and concentrated to give 0.40 g (66%) of the title compound as a brown oil.

### Step 2

### 3-methyl-N-{[3-({[(2,4,6-trimethylphenyl)amino]carbonyl}amino)-2-naphthalenyl]carbonyl}-L-valine

A solution of N-[(3-amino-2-naphthalenyl)carbonyl]-3-methyl-L-valine (0.23 g, 0.74 mmol) and 2-isocyanato-1,3,5-trimethylbenzene (0.13 g, 0.81 mmol) in dry pyridine (3 mL) was stirred at RT for 16 h, then concentrated to dryness. A 1N HCl solution and ethyl acetate were added. The organic layer was washed with brine solution, dried over MgSO₄, filtered and concentrated. The solid was dissolved in MeOH (2 mL) and THF (2 mL) to which LiOH (0.18 g, 7.40 mmol) in water (5 mL) was added. After 3 h, TLC shows no remaining starting material. A 1N HCl solution and ethyl acetate were added. The organic layer was dried over MgSO₄, filtered and concentrated. The crude material was purified by reverse-phase HPLC (Gilson: MeCN, 1% TFA/water) to give 0.060 g (18%) of the title compound as a white solid. ES MS m/z 460 (M−H).

### Example 336

### (3R)-3-phenyl-3-({[3-({[(2,4,6-trimethylphenyl)amino]carbonyl}amino)-2-naphthalenyl]carbonyl}amino)propanoic acid

### Step 1

### 1,1-dimethylethyl (3R)-3-{[(3-amino-2-naphthalenyl)carbonyl]amino}-3-phenylpropanoate

HATU (0.47 g, 1.24 mmol) was added to a mixture of 3-amino-2-naphthalenecarboxylic acid (0.21 g, 1.13 mmol), 1,1-dimethylethyl (3R)-3-amino-3-phenylpropanoate (0.30 g, 1.36 mmol) and N,N-diisopropylethylamine (0.40 mL, 2.26 mmol) in DMF (10 mL). The solution was stirred at RT for 2 h then saturated NaHCO₃ solution and ethyl acetate were added. The organic layer was washed with water, brine, dried over MgSO₄, filtered and concentrated to give 0.42 g (94%) of the title compound as a brown oil.

### Step 2

### (3R)-3-phenyl-3-({[3-({[(2,4,6-trimethylphenyl)amino]carbonyl}amino)-2-naphthalenyl]carbonyl}amino)propanoic acid

A solution of 1,1-dimethylethyl (3R)-3-{[(3-amino-2-naphthalenyl)carbonyl]amino}-3-phenylpropanoate (0.38 g, 0.97 mmol) and 2-isocyanato-1,3,5-trimethylbenzene (0.17 g, 1.07 mmol) in dry pyridine (5 mL) were stirred at RT for 16 h. Water and 1N HCl (3 mL) were added followed by ethyl acetate. The organic layer was washed with water, brine, dried over MgSO₄, filtered and concentrated. The crude material was purified on silica gel using an ISCO chromatography system (gradient: 100% hexanes to 100% ethyl acetate over 25 minutes) to give an orange solid. The solid was dissolved in DCM and TFA (2 mL) was added. The solution was heated to reflux until no starting material remained then the solvent was removed via rotary evaporation. The crude material was purified by reverse-phase HPLC (Gilson: MeCN, 1% TFA/water) to give 0.064 g (13% over two steps) of the title compound as a white solid. ES MS m/z 494 (M−H).

### Example 337

### 1,1-dimethylethyl (3R)-3-cyclohexyl-3-({[3-({[(2,4,6-trimethylphenyl)amino]carbonyl}amino)-2-naphthalenyl]carbonyl}amino)propanoate

### Step 1

### 1,1-dimethylethyl (3R)-3-amino-3-cyclohexylpropanoate

A mixture of 1,1-dimethylethyl (3R)-3-amino-3-phenylpropanoate (0.50 g, 2.26 mmol) and Rh/Al₂O₃ (0.10 g) in MeOH (10 mL) under hydrogen (60 psig) was heated to 80° C. for 24 h. The reaction was cooled to RT, carefully vented, and then filtered through Celite. The filtrate was concentrated to give 0.41 g (80%) of the title compound as a yellow oil.

### Step 2

### 1,1-dimethylethyl (3R)-3-{[(3-amino-2-naphthalenyl)carbonyl]amino}-3-cyclohexylpropanoate

HATU (0.96 g, 2.53 mmol) was added to a solution of 3-amino-2-naphthalenecarboxylic acid (0.34 g, 1.81 mmol), 1,1-dimethylethyl (3R)-3-amino-3-cyclohexylpropanoate (0.41 g, 1.81 mmol) and N,N-diisopropylethylamine (0.69 mL, 3.98 mmol) in DMF (10 mL). The solution was stirred at RT for 48 h then saturated NaHCO₃ solution and ethyl acetate were added. The organic layer was washed with saturated NaHCO₃ solution, brine, dried over MgSO₄, filtered and concentrated. The crude product was purified on silica gel using an ISCO chromatography system (gradient: 100% hexanes to 100% ethyl acetate over 25 minutes) to give 0.57 g (80%) of the title compound as a yellow solid.

### Step 3

### 1,1-dimethylethyl (3R)-3-cyclohexyl-3-({[3-({[(2,4,6-trimethylphenyl)amino]carbonyl}amino)-2-naphthalenyl]carbonyl}amino)propanoate

A solution of 1,1-dimethylethyl (3R)-3-{[(3-amino-2-naphthalenyl)carbonyl]amino}-3-cyclohexylpropanoate (0.17 g, 0.43 mmol) and 2-isocyanato-1,3,5-trimethylbenzene (0.084 g, 0.52 mmol) in dry pyridine (3 mL) were stirred at RT for 16 h then concentrated to dryness via rotary evaporation. 1N HCl and ethyl acetate were added. The organic layer was washed with 1N HCl, brine, dried over MgSO₄, filtered and concentrated. The crude material was purified on silica gel using an ISCO chromatography system (gradient: 100% hexanes to 100% ethyl acetate over 25 minutes) to give 0.13 g (54%) of the title compound as an orange solid. ES MS m/z 556 (M−H).

### Example 338

### (3R)-3-cyclohexyl-3-({[3-({[(2,4,6-trimethylphenyl)amino]carbonyl}amino)-2-naphthalenyl]carbonyl}amino)propanoic acid

1,1-dimethylethyl (3R)-3-cyclohexyl-3-({[3-({[(2,4,6-trimethylphenyl)amino]carbonyl}amino)-2-naphthalenyl]carbonyl}amino)propanoate (0.11 g, 0.19 mmol) was dissolved in DCM and TFA (1.5 mL) was added. The solution was stirred for 6 h then concentrated to dryness via rotary evaporation. The crude material was dissolved in a minimal amount of DCM then triturated with Et₂O and hexanes. The solid was filtered and dried under vacuum to give 0.077 g (81%) of the title compound as a white powder. ES MS m/z 500 (M−H).

### Example 339

### (3R)-4-methyl-3-({[3-({[(2,4,6-trimethylphenyl)amino]carbonyl}amino)-2-naphthalenyl]carbonyl}amino)pentanoic acid

### Step 1

### 1,1-dimethylethyl[(1S)-1-(iodomethyl)-2-methylpropyl]carbamate

Iodine (8.74 g, 34.7 mmol) was added to a suspension of polystyrene-supported diphenylphosphine (15.8 g, 34.7 mmol) in DCM (300 mL), After 15 min, imidazole (2.7 g, 39.5 mmol) was added and after 15 min a solution of 1,1-dimethylethyl[(1S)-1-(hydroxymethyl)-2-methylpropyl]carbamate (3.2 g, 15.8 mmol) in DCM (60 mL) was added. The mixture was stirred overnight then filtered through Celite. The solution was washed with 0.5M Na₂S₂O₃ solution, water, dried over MgSO₄, filtered and concentrated to give 2.3 g of the title compound with some unreacted 1,1-dimethylethyl[(1S)-1-(hydroxymethyl)-2-methylpropyl]carbamate impurity. The material was used without further purification.

### Step 2

### 1,1-dimethylethyl[(1R)-1-(cyanomethyl)-2-methylpropyl]carbamate

A solution of 1,1-dimethylethyl[(1S)-1-(iodomethyl)-2-methylpropyl]carbamate (2.3 g) and tetraethylammonium cyanide (1.26 g, 8.09 mmol) in DCM (100 mL) was heated to reflux for 4 h then concentrated to dryness. The crude material was purified on silica gel using an ISCO chromatography system (100% hexanes to 100% ethyl acetate over 25 min) to give 0.67 g (20% over two steps) of the title compound as a white solid.

### Step 3

### methyl(3R)-3-amino-4-methylpentanoate hydrochloride

HCl gas was bubbled into MeOH (10 mL) at 0° C. until saturated and then 1,1-dimethylethyl[(1R)-1-(cyanomethyl)-2-methylpropyl]carbamate (0.60 g, 2.83 mmol) was added. A little more HCl gas was bubbled in then the tube was sealed at stirred at RT for 16 h. The vessel was vented and water (ca. 7 drops) was added. The solution was stirred for 1 h then concentrated. Et₂O and MeOH were added and the solution was concentrated to dryness. Et₂O was again added and the solution concentrated to give 0.63 g of the title compound as a white solid.

### Step 4

### methyl(3R)-3-{[(3-amino-2-naphthalenyl)carbonyl]amino}-4-methylpentanoate

HATU (1.43 g, 3.77 mmol) was added to a suspension of 3-amino-2-naphthalenecarboxylic acid (0.59 g, 3.14 mmol), methyl(3R)-3-amino-4-methylpentanoate hydrochloride (0.63 g, 3.46 mmol) and N,N-diisopropylethylamine (1.20 mL, 6.91 mmol) in DMF (6 mL). The solution was stirred at RT for 6 h then ethyl acetate and saturated NaHCO₃ solution were added. The organic layer was washed with saturated NaHCO₃ solution, brine, dried over MgSO₄, filtered and concentrated. The crude material was purified on silica gel using an ISCO chromatography system (gradient: 100% hexanes to 100% ethyl acetate over 25 minutes) to give 0.25 g (25%) of the title compound as a yellow solid.

### Step 5

### (3R)-4-methyl-3-({[3-({[(2,4,6-trimethylphenyl)amino]carbonyl}amino)-2-naphthalenyl]carbonyl}amino)pentanoic acid

A solution of methyl(3R)-3-{[(3-amino-2-naphthalenyl)carbonyl]amino}-4-methylpentanoate (0.19 g, 0.60 mmol) and 2-isocyanato-1,3,5-trimethylbenzene (0.115 g, 0.71 mmol) in dry pyridine (3 mL) was stirred at RT for 24 h then concentrated to dryness via rotary evaporation. 1N HCl and ethyl acetate were added. The organic layer was washed with 1N HCl, brine, dried over MgSO₄, filtered and concentrated. The crude material was dissolved in THF (ca. 2 mL) and MeOH (ca. 2 mL) and a solution of LiOH (0.20 g, 8.33 mmol) in water (5 mL) was added. The mixture was stirred until no starting material remained as evident from TLC then 1N HCl and ethyl acetate were added. The organic layer was washed with brine, dried over MgSO₄, filtered and concentrated. The crude material was purified by reverse-phase HPLC (Gilson: MeCN, 1% TFA/water) to give 0.11 g (40% over two steps) of the title compound as a white solid. ES MS m/z 460 (M−H).

### Example 340

### N-[(1S)-2-methyl-1-(1H-tetrazol-5-yl)propyl]-3-({[(2,4,6-trimethylphenyl)amino]carbonyl}amino)-2-naphthalenecarboxamide

### Step 1

### [(1S)-2-methyl-1-(1H-tetrazol-5-yl)propyl]amine trifluoroacetate

A mixture of 1,1-dimethylethyl[(1S)-1-cyano-2-methylpropyl]carbamate (2.00 g, 10.09 mmol), sodium azide (1.30 g, 20.20 mmol) and ZnBr₂ (1.14 g, 5.05 mmol) in water (30 mL) and iPrOH (15 mL) were heated to 80° C. for 16 h then cooled to RT. Ethyl acetate (30 mL) and 3N HCl (5 mL) were added. The aqueous layer was extracted again with ethyl acetate and the organic layers were combined and concentrated to dryness via rotary evaporation to give a white solid. ¹H NMR showed a mixture of the desired product and starting material (nitrile). Therefore this material was re-dissolved in water (30 mL) and iPrOH (15 mL) then sodium azide (1.30 g, 20.20 mmol) and ZnBr₂ (1.14 g, 5.05 mmol) were added. The mixture was heated to 100° C. for 6 h then the mixture was cooled to RT. Ethyl acetate (30 mL) and 3N HCl (5 mL) were added. The aqueous layer was extracted again with ethyl acetate and the organic layers were combined and concentrated to dryness via rotary evaporation to give a white solid. The material (ca. 0.50 g) was dissolved in DCM and TFA (1 mL) was added. After 2 h, the solution was concentrated to dryness via rotary evaporation to 0.20 g (8%) the title compound.

### Step 2

### 3-amino-N-[(1S)-2-methyl-1-(1H-tetrazol-5-yl)propyl]-2-naphthalenecarboxamide

HATU (0.33 g, 0.86 mmol) was added to a suspension of 3-amino-2-naphthalenecarboxylic acid (0.15 g, 0.80 mmol), [(1S)-2-methyl-1-(1H-tetrazol-5-yl)propyl]amine trifluoroacetate (0.20 g, 0.78 mmol) and N,N-diisopropylethylamine (0.4 mL, 2.34 mmol) in DMF (10 mL). The solution was stirred at RT for 2 h then ethyl acetate and brine solution were added. The organic layer was dried over MgSO₄, filtered and concentrated. The crude material was stirred with 2-isocyanato-1,3,5-trimethylbenzene (0.15 g, 0.93 mmol) in dry pyridine (3 mL) at RT for 3 h then concentrated to dryness via rotary evaporation. 1N HCl and ethyl acetate were added. The organic layer was washed with brine, dried over MgSO₄, filtered and concentrated. The crude material was purified by reverse-phase HPLC (Gilson: MeCN, 1% TFA/water) to give 0.06 g (16% over two steps) of the title compound as a tan solid. ES MS m/z 470 (M−H).

### Example 341

### (2S)-(4,4-difluorocyclohexyl)({[3-({[(2,4,6-trimethylphenyl)amino]carbonyl}amino)-2-naphthalenyl]carbonyl}amino)ethanoic acid And

### Example 342

### N—[(S)-cyano(4,4-difluorocyclohexyl)methyl]-3-({[(2,4,6-trimethylphenyl)amino]carbonyl}amino)-2-naphthalenecarboxamide

### Step 1

### (4,4-difluorocyclohexyl)methanol

A solution of 4,4-difluorocyclohexanecarboxylic acid (2.00 g, 12.20 mmol) in THF (6 mL) was added dropwise to NaBH₄ (0.46 g, 12.20 mmol) in THF (10 mL) at 0° C. After stirring for 1 h, neat BF₃-Et₂O (1.54 mL, 12.20 mmol) was added dropwise and the resulting white slurry was stirred at RT overnight. EtOH (12 mL) was added slowly and the mixture was stirred for 0.5 h then concentrated via rotary evaporation. DCM (300 mL) and water (300 mL) were added. The aqueous layer was extracted again with DCM and the combined organic layers were dried over MgSO₄, filtered and concentrated to give 2.1 g (115%) of the title compound as a clear oil. Note: although some DCM still remained (as evident from ¹H NMR) it was not concentrated excessively due to the potential volatility of the product.

### Step 2

### 4,4-difluorocyclohexanecarbaldehyde

Dess-Martin periodinane (7.21 g, 17.00 mmol) was added as a solid to a solution of (4,4-difluorocyclohexyl)methanol (1.70 g, 11.30 mmol) in DCM (150 mL) at −780° C. The mixture was allowed to warm to RT and water (ca. 3 drops) was added. After 3 h at RT, the mixture was poured into a 1:1 mixture of saturated NaHCO₃ and Na₂S₂O₃ solutions (90 mL each) and then stirred for 0.5 h. The organic layer was separated, washed with brine, dried over MgSO₄, filtered and concentrated to give 1.70 g (100%) of the title compound.

### Step 3

### (S)—N-[(1E)-(4,4-difluorocyclohexyl)methylidene]-4-methylbenzenesulfinamide

Titanium (IV) ethoxide (12.0 mL, 57.5 mmol) was added to a solution of 4,4-difluorocyclohexanecarbaldehyde (1.7 g, 11.5 mmol) and (S)-4-methylbenzenesulfinamide (1.78 g, 11.5 mmol) in DCM (25 mL). The yellow solution was heated to reflux for 5 h then cooled to RT. Water (15 mL) was added and the thick slurry was stirred with a spatula. DCM was added and the solid was filtered off and washed with DCM. The combined organic layers were washed with water (3×'s), brine, dried over MgSO₄, filtered and concentrated via rotary evaporation at RT to give 2.44 g (75%) of the title compound.

### Step 4

### N—[(S)-cyano(4,4-difluorocyclohexyl)methyl]-(S)-4-methylbenzenesulfinamide

A 1N solution of diethylaluminum cyanide (12.9 mL, 12.9 mmol) in toluene was added to a solution of iPrOH (0.65 mL, 8.49 mmol) in THF (100 mL) at RT. After 0.5 h, the solution was cooled to −78° C. and (S)—N-[(1E)-(4,4-difluorocyclohexyl)methylidene]-4-methylbenzenesulfinamide (2.44 g, 8.59 mmol) in THF (200 mL) was added. The reaction was warmed to RT and stirred for 3.5 h, after which saturated NH₄Cl solution (4 mL) was added followed by water (200 mL) and ethyl acetate (200 mL). The mixture was filtered through Celite and the organic layer was separated, washed with brine, dried over MgSO₄, filtered and concentrated. The crude material was purified on silica gel using an ISCO chromatography system (gradient: 100% hexanes to 100% ethyl acetate over 25 minutes) to give 0.70 g (26%) of the title compound as a white solid. ¹H NMR shows a 13:1 mixture of diastereomers (84% d.e.).

### Step 5

### Mixture of methyl(2S)-amino(4,4-difluorocyclohexyl)ethanoate hydrochloride and (2S)-amino(4,4-difluorocyclohexyl)ethanenitrile hydrochloride

HCl gas was bubbled into a solution of N—[(S)-cyano(4,4-difluorocyclohexyl)methyl]-(S)-4-methylbenzenesulfinamide (0.70 g, 2.24 mmol) in MeOH (15 mL) and water (0.25 mL) in a sealable tube until saturated. The tube was sealed and heated to 100° C. for 24 h. The vessel was cooled to RT, carefully vented, and the solution was concentrated. Ethyl acetate and saturated NaHCO₃ solution were added. The organic layer was dried over MgSO₄, filtered and concentrated The crude material was converted to the HCl salt with 1N HCl in Et₂O and the solid was washed with hexanes/Et₂O and dried under vacuum to give 0.41 g (75%) of a mixture of the title compounds as a white solid.

### Step 6

### Mixture of methyl(2S)-{[(3-amino-2-naphthalenyl)carbonyl]amino}(4,4-difluorocyclohexyl)ethanoate and 3-amino-N—[(S)-cyano(4,4-difluorocyclohexyl)methyl]-2-naphthalenecarboxamide

A mixture of methyl(2S)-amino(4,4-difluorocyclohexyl)ethanoate hydrochloride and (2S)-amino(4,4-difluorocyclohexyl)ethanenitrile hydrochloride (0.35 g, 1.44 mmol) was dissolved in DMF (5 mL). Next, 3-amino-2-naphthalenecarboxylic acid (0.27 g, 1.44 mmol) and N,N-diisopropylethylamine (1.5 mL, 8.6 mmol) were added followed by HATU (0.77 g, 2.02 mmol). The solution was stirred at RT for 24 h then ethyl acetate and saturated NaHCO₃ solution were added. The organic layer was washed with brine, dried over MgSO₄, filtered and concentrated to give 0.66 g of a mixture of the title compounds.

### Step 7

### Mixture of methyl(2S)-(4,4-difluorocyclohexyl)({[3-({[(2,4,6-trimethylphenyl)amino]carbonyl}amino)-2-naphthalenyl]carbonyl}amino)ethanoate and N—[(S)-cyano(4,4-difluorocyclohexyl)methyl]-3-({[(2,4,6-trimethylphenyl)amino]carbonyl}amino)-2-naphthalenecarboxamide

A mixture of methyl(2S)-{[(3-amino-2-naphthalenyl)carbonyl]amino}(4,4-difluorocyclohexyl)ethanoate and 3-amino-N—[(S)-cyano(4,4-difluorocyclohexyl)methyl]-2-naphthalenecarboxamide (0.66 g, 1.76 mmol) was stirred with 2-isocyanato-1,3,5-trimethylbenzene (0.34 g, 2.11 mmol) in dry pyridine (3 mL) at RT for 4 h then concentrated to dryness via rotary evaporation. 1N HCl and ethyl acetate were added. The organic layer was washed with 1N HCl, brine, dried over MgSO₄, filtered and concentrated to give 0.40 g (42%) of a mixture of the title compounds.

### Step 8

### (2S)-(4,4-difluorocyclohexyl)({[3-({[(2,4,6-trimethylphenyl)amino]carbonyl}amino)-2-naphthalenyl]carbonyl}amino)ethanoic acid and

### N—[(S)-cyano(4,4-difluorocyclohexyl)methyl]-3-({[(2,4,6-trimethylphenyl)amino]carbonyl}amino)-2-naphthalenecarboxamide

A mixture of methyl(2S)-(4,4-difluorocyclohexyl)({[3-({[(2,4,6-trimethylphenyl)amino]carbonyl}amino)-2-naphthalenyl]carbonyl}amino)ethanoate and N—[(S)-cyano(4,4-difluorocyclohexyl)methyl]-3-({[(2,4,6-trimethylphenyl)amino]carbonyl}amino)-2-naphthalenecarboxamide (0.40 g, 0.74 mmol) was dissolved in THF (ca. 2 mL) and MeOH (ca. 2 mL) and a solution of LiOH (0.10 g, 4.17 mmol) in water (5 mL) was added. The mixture was stirred until no starting material remained as evident from LC/MS (ca. 4 h) then 1N HCl and ethyl acetate were added. The organic layer was washed with brine, dried over MgSO₄, filtered and concentrated. The crude material was purified on silica gel using an ISCO chromatography system (gradient: 100% hexanes to 100% ethyl acetate to 5% MeOH/ethyl acetate) to give two products: 0.26 g (68%) of (2S)-(4,4-difluorocyclohexyl)({[3-({[(2,4,6-trimethylphenyl)amino]carbonyl}amino)-2-naphthalenyl]carbonyl}amino)ethanoic acid. ES MS m/z 522 (M−H). and

0.15 g (40%) of N—[(S)-cyano(4,4-difluorocyclohexyl)methyl]-3-({[(2,4,6-trimethylphenyl)amino]carbonyl}amino)-2-naphthalenecarboxamide. ES MS m/z 503 (M−H).

### Example 343

### (2S)-cyclopentyl({[3-({[(2,4,6-trichlorophenyl)amino]carbonyl}amino)-2-naphthalenyl]carbonyl}amino)ethanoic acid

### Step 1

### methyl(2S)-amino(cyclopentyl)ethanoate trifluoroacetate

The dicyclohexylamine salt of (2S)-cyclopentyl({[(1,1-dimethylethyl)oxy]carbonyl}amino)ethanoic acid (2.0 g, 4.72 mmol) was dissolved in ethyl acetate and converted to the free acid with 1N HCl solution. The organic layer was separated, dried over MgSO₄, filtered and concentrated. This material (1.0 g, 4.1 mmol) was dissolved in ethyl acetate (15 mL) and methanol (15 mL) then a 2M solution of (trimethylsilyl)diazomethane in hexanes (4.11 mL, 8.23 mmol) was added dropwise. After 4 h, the yellow solution was concentrated to dryness. The crude material was dissolved in DCM and TFA (2 mL) was added. After 1.5 h, the solution was concentrated to dryness to give 0.82 (74%) of the title compound as a yellow oil.

### Step 2

### methyl(2S)-{[(3-amino-2-naphthalenyl)carbonyl]amino}(cyclopentyl)ethanoate

HATU (1.62 g, 4.24 mmol) was added to a suspension of 3-amino-2-naphthalenecarboxylic acid (0.57 g, 3.03 mmol), methyl(2S)-amino(cyclopentyl)ethanoate trifluoroacetate (0.82 g, 3.03 mmol) and N,N-diisopropylethylamine (2.0 mL, 11.5 mmol) in DMF (10 mL). The solution was stirred at RT for 16 h then ethyl acetate and saturated NaHCO₃ solution were added. The organic layer was washed with saturated NaHCO₃ solution, brine, dried over MgSO₄, filtered and concentrated. The crude material was purified on silica gel using an ISCO chromatography system (gradient: 100% hexanes to 50% ethyl acetate/hexanes) to give 0.60 g (61%) of the title compound as an orange solid.

### Step 3

### methyl(2S)-cyclopentyl({[3-({[(2,4,6-trichlorophenyl)amino]carbonyl}amino)-2-naphthalenyl]carbonyl}amino)ethanoate

A solution of methyl(2S)-{[(3-amino-2-naphthalenyl)carbonyl]amino}(cyclopentyl)ethanoate (0.21 g, 0.64 mmol) and 1,3,5-trichloro-2-isocyanatobenzene (0.157 g, 0.71 mmol) in dry pyridine (3 mL) were stirred at RT for 16 h then concentrated to dryness via rotary evaporation. 1N HCl and ethyl acetate were added. The organic layer was washed with water, brine, dried over MgSO₄, filtered and concentrated to give 0.31 g (89%) of the title compound as a white solid.

### Step 3

### (2S)-cyclopentyl({[3-({[(2,4,6-trichlorophenyl)amino]carbonyl}amino)-2-naphthalenyl]carbonyl}amino)ethanoic acid

LiOH (0.30 g, 12.50 mmol) in hot water (10 mL) was added to a solution of methyl(2S) -cyclopentyl({[3-({[(2,4,6-trichlorophenyl)amino]carbonyl}amino)-2-naphthalenyl]carbonyl}amino)ethanoate (0.26 g, 0.47 mmol) in THF (10 mL) and MeOH (5 mL). The mixture was stirred until no starting material remained as evident from TLC then 1N HCl and ethyl acetate were added. The organic layer was washed with brine, dried over MgSO₄, filtered and concentrated. The crude material was dissolved in hot EtOH and hexane was added. The solution was allowed to cool to RT overnight then the resulting solid was filtered and dried under vacuum to give 0.23 g (94%) of the title compound as a white solid. ES MS m/z 533 (M−H).

### Example 344

### (2S)-cyclopentyl({[3-({[(2,4,6-trimethylphenyl)amino]carbonyl}amino)-2-naphthalenyl]carbonyl}amino)ethanoic acid

### Step 1

### methyl(2S)-amino(cyclopentyl)ethanoate hydrochloride

(2S)-cyclopentyl({[(1,1-dimethylethyl)oxy]carbonyl}amino)ethanoic acid dicyclohexylamine (2.9 g, 6.84 mmol) was dissolved in ethyl acetate (400 mL) and washed with 1N HCl (200 mL). The organic layer was dried over MgSO₄, filtered and concentrated to give (2S)-cyclopentyl({[(1,1-dimethylethyl)oxy]carbonyl}amino)ethanoic acid as the free acid. The clear oil was dissolved in ethyl acetate (25 mL) and MeOH (25 mL) and a 2M solution of TMS-diazomethane in Et₂O (6.84 mL, 13.68 mmol) was added slowly. (Note: yellow color persists at the end of the addition). The solution was stirred overnight then concentrated to dryness to give a white solid which was dissolved in CH₂Cl₂ (30 mL) and TFA (5 mL). After 3 h the solution was concentrated to give 2.2 g (119%) of the title compound as a yellow oil. Some of the material was converted to the HCl salt in an attempt to solidify the compound.

### Step 2

### methyl(2S)-{[(3-amino-2-naphthalenyl)carbonyl]amino}(cyclopentyl)ethanoate

HATU (2.57 g, 6.77 mmol) was added to a suspension of 3-amino-2-naphthalenecarboxylic acid (0.99 g, 4.51 mmol), methyl(2S)-amino(cyclopentyl)ethanoate hydrochloride (0.87 g, 4.51 mmol), and N,N-diisopropylethylamine (3.25 mL, 13.53 mmol) in DMF (15 mL) at RT. After 3 h, saturated NaHCO₃ solution (100 mL) and ethyl acetate (200 mL) were added. The organic layer was washed with brine (100 mL), dried over MgSO₄, filtered and concentrated. The crude product was purified on silica gel using an ISCO chromatography system (increasing gradient from 100% hexanes to 90% ethyl acetate/hexanes over 40 min) to give 0.75 g (51%) of the title compound as a yellow solid (ca. 80% pure).

### Step 3

### methyl(2S)-cyclopentyl({[3-({[(2,4,6-trimethylphenyl)amino]carbonyl}amino)-2-naphthalenyl]carbonyl}amino)ethanoate

A solution of methyl(2S)-{[(3-amino-2-naphthalenyl)carbonyl]amino}(cyclopentyl)ethanoate (0.55 g, 1.69 mmol) and 2-isocyanato-1,3,5-trimethylbenzene (0.54 g, 3.37 mmol) in pyridine (5 mL) were stirred at RT for 4 h. The solvent was removed under reduced pressure and ethyl acetate (100 mL) and 1N HCl (100 mL) were added. The organic layer was washed with water (100 mL), brine (100 mL), dried over MgSO₄, filtered and concentrated to give a pale yellow solid (ca. 80% pure by ¹H NMR). The crude material was purified on silica gel (ISCO chromatography: 100% hexanes to 80% ethyl acetate/hexanes) to give 0.48 g (58%) of a yellow solid.

### Step 4

### (2S)-cyclopentyl({[3-({[(2,4,6-trimethylphenyl)amino]carbonyl}amino)-2-naphthalenyl]carbonyl}amino)ethanoic acid

LiOH (0.20 g, 8.33 mmol) in hot water (25 mL) was added to a solution of methyl(2S) -cyclopentyl({[3-({[(2,4,6-trimethylphenyl)amino]carbonyl}amino)-2-naphthalenyl]carbonyl}amino)ethanoate (0.48 g, 0.98 mmol) in MeOH/THF (1:1, 10 mL). The reaction was stirred until no starting material remained then 1N HCl and ethyl acetate were added. The organic layer was washed with brine, dried over MgSO₄, filtered and concentrated. The crude material was purified by reverse-phase HPLC (Gilson: MeCN, 1% TFA/water) to give 0.083 g (18%) of the title compound as a white solid. ES MS m/z 472 (M−H).

### Example 345

### 1,4-dioxaspiro[4.5]dec-8-yl({[3-({[(2,4,6-trimethylphenyl)amino]carbonyl}amino)-2-naphthalenyl]carbonyl}amino)acetic acid

### Step 1

### methyl 1,4-dioxaspiro[4.5]dec-8-ylidene({[(phenylmethyl)oxy]carbonyl}amino)acetate

DBU (3.8 mL, 25.4 mmol) was added dropwise to a solution of 1,4-dioxaspiro[4.5]decan-8-one (3.31 g, 21.2 mmol) and methyl[bis(methyloxy)phosphoryl]({[(phenylmethyl)oxy]carbonyl}amino)acetate (7.02 g, 21.20 mmol) in DCM (50 mL) at RT. After 3 days, the brown solution was concentrated to dryness and ethyl acetate and water were added. The organic layer was washed with water, brine, dried over MgSO₄, filtered and concentrated to give 5.6 g (73%) of the title compound as an orange oil.

### Step 2

### methyl amino(1,4-dioxaspiro[4.5]dec-8-yl)acetate

A mixture of methyl 1,4-dioxaspiro[4.5]dec-8-ylidene({[(phenylmethyl)oxy]carbonyl}amino)acetate (5.2 g, 14.4 mmol) and 10% Pd/C (0.2 g) in MeOH (50 mL) was stirred under H₂ (60 psig) overnight. The next day, the reaction was carefully vented, diluted with ethyl acetate, filtered through Celite, and concentrated to dryness. The crude material was dissolved in hot EtOH and hexane was added slowly until cloudy. The solution was allowed to cool slowly to RT and the resulting solid was filtered and dried under vacuum to give 2.6 g (79%) of the title compound as an off-white solid. ¹H NMR shows material is only ca. 70% pure.

### Step 3

### methyl {[(3-amino-2-naphthalenyl)carbonyl]amino}(1,4-dioxaspiro[4.5]dec-8-yl)acetate

HATU (1.53 g, 4.01 mmol) was added to a suspension of 3-amino-2-naphthalenecarboxylic acid (0.50 g, 2.67 mmol), methyl amino(1,4-dioxaspiro[4.5]dec-8-yl)acetate (0.61 g, 2.67 mmol), and N,N-diisopropylethylamine (1.9 mL, 8.01 mmol) in DMF (15 mL) at RT. After 5 h, saturated NaHCO₃ solution (100 mL) and ethyl acetate (200 mL) were added. The organic layer was washed with brine (100 mL), dried over MgSO₄, filtered and concentrated. The crude product was purified on silica gel using an ISCO chromatography system (increasing gradient from 100% hexanes to 90% ethyl acetate/hexanes over 40 min) to give 0.37 g (34%) of the title compound as a yellow solid. ¹H NMR shows material is only ca. 85% pure.

### Step 4

### 1,4-dioxaspiro[4.5]dec-8-yl({[3-({[(2,4,6-trimethylphenyl)amino]carbonyl}amino)-2-naphthalenyl]carbonyl}amino)acetic acid

A solution of methyl {[(3-amino-2-naphthalenyl)carbonyl]amino}(1,4-dioxaspiro[4.5]dec-8-yl)acetate (0.37 g, 0.92 mmol) and 2-isocyanato-1,3,5-trimethylbenzene (0.18 g, 1.11 mmol) in pyridine (5 mL) were stirred at RT for 5 h. The solvent was removed under reduced pressure and ethyl acetate (100 mL) and 0.1 N HCl (100 mL) were added. The organic layer was washed with water (100 mL), brine (100 mL), dried over MgSO₄, filtered and concentrated to give a pale yellow solid. The crude material was dissolved in THF (10 mL) and MeOH (10 mL) then a hot solution of LiOH (0.30 g, 12.50 mmol) in water (25 mL) was added. The yellow solution was stirred until no starting material remained as evident by TLC (100% ethyl acetate) then ethyl acetate (200 mL) and 1N HCl solution (100 mL) were added. The organic layer was washed with brine (100 mL), dried over MgSO₄, filtered and concentrated to give 0.46 g of yellow solid (ca.-85% purity). A portion of the crude material (−0.075 g) was purified by reverse phase HPLC (Gilson: MeCN, 1% TFA/water) to give 0.070 g of the title compound as a pinkish solid. ES MS m/z 544 (M−H).

### Example 346

### (4-oxocyclohexyl)({[3-({[(2,4,6-trimethylphenyl)amino]carbonyl}amino)-2-naphthalenyl]carbonyl}amino)acetic acid

Pyridinium p-toluenesulfonate (ca. 100 mg) was added to a solution of 1,4-dioxaspiro[4.5]dec-8-yl({[3-({[(2,4,6-trimethylphenyl)amino]carbonyl}amino)-2-naphthalenyl]carbonyl}amino)acetic acid (0.40 g, 0.73 mmol) in acetone (10 mL) and water (10 mL) and the solution was heated at 70° C. After 24 h, TLC and LCMS show no starting material remaining so 1N HCl (100 mL) and ethyl acetate (100 mL) were added. The organic layer was washed with brine (100 mL), dried over MgSO₄, filtered and concentrated to give 0.28 g of yellow solid (85% purity by ¹H NMR). Reverse phase HPLC (Gilson: MeCN, 1% TFA/water) gave 0.17 g (46%) of the title compound as a white solid. ES MS m/z 500 (M−H).

### Example 347

### (cis and trans)-[4-(cyclobutylamino)cyclohexyl]({[3-({[(2,4,6-trimethylphenyl)amino]carbonyl}amino)-2-naphthalenyl]carbonyl}amino)acetic acid

### Step 1

### methyl {[(3-amino-2-naphthalenyl)carbonyl]amino}(1,4-dioxaspiro[4.5]dec-8-yl)acetate

HATU (2.04 g, 5.37 mmol) was added to a suspension of 3-amino-2-naphthalenecarboxylic acid (0.67 g, 3.58 mmol), methyl amino(1,4-dioxaspiro[4.5]dec-8-yl)acetate (0.82 g, 3.58 mmol), and N,N-diisopropylethylamine (2.6 mL, 10.7 mmol) in DMF (50 mL) at RT. After 5 h, saturated NaHCO₃ solution (200 mL) and ethyl acetate (200 mL) were added. The organic layer was washed with brine (100 mL), dried over MgSO₄, filtered and concentrated. The crude product was purified on silica gel using an ISCO chromatography system (increasing gradient from 100% hexanes to 90% ethylacetate/hexanes over 40 min) to give 1.02 g (71%) of the title compound as a yellow solid.

### Step 2

### methyl 1,4-dioxaspiro[4.5]dec-8-yl({[3-({[(2,4,6-trimethylphenyl)amino]carbonyl}amino)-2-naphthalenyl]carbonyl}amino)acetate

A solution of methyl {[(3-amino-2-naphthalenyl)carbonyl]amino}(1,4-dioxaspiro[4.5]dec-8-yl)acetate (0.95 g, 2.38 mmol) and 2-isocyanato-1,3,5-trimethylbenzene (0.78 g, 4.77 mmol) in pyridine (20 mL) were stirred at RT for 5 h. The solvent was removed under reduced pressure and ethyl acetate (200 mL) and 0.1 N HCl (200 mL) were added. The organic layer was washed with water (100 mL), brine (100 mL), dried over MgSO₄, filtered and concentrated to give 1.17 g (88%) of a pale yellow solid.

### Step 3

### 1,4-dioxaspiro[4.5]dec-8-yl({[3-({[(2,4,6-trimethylphenyl)amino]carbonyl}amino)-2-naphthalenyl]carbonyl}amino)acetic acid

LiOH (0.80 g, 33.33 mmol) was dissolved in hot water (25 mL) and added while still hot to a solution of methyl 1,4-dioxaspiro[4.5]dec-8-yl({[3-({[(2,4,6-trimethylphenyl)amino]carbonyl}amino)-2-naphthalenyl]carbonyl}amino)acetate (0.69 g, 1.23 mmol) in THF (10 mL) and MeOH (10 mL). The yellow solution was stirred until no starting material remained as evident by TLC (100% ethyl acetate) then ethyl acetate (300 mL) and 1N HCl solution (100 mL) were added. The organic layer was washed with brine (100 mL), dried over MgSO₄, filtered and concentrated to give 0.66 g (98%) of the title compound as a yellow solid.

### Step 4

### (4-oxocyclohexyl)({[3-({[(2,4,6-trimethylphenyl)amino]carbonyl}amino)-2-naphthalenyl]carbonyl}amino)acetic acid

Pyridinium p-toluenesulfonate (ca. 200 mg) was added to a solution of 1,4-dioxaspiro[4.5]dec-8-yl({[3-({[(2,4,6-trimethylphenyl)amino]carbonyl}amino)-2-naphthalenyl]carbonyl}amino)acetic acid (0.65 g, 1.19 mmol) in acetone (20 mL) and water (20 mL) and the solution was heated at 70° C. for 24 h. 1N HCl (100 mL) and ethyl acetate (200 mL) added. The organic layer was washed with brine (200 mL), dried over MgSO₄, filtered and concentrated to give 0.56 g (94%) of the title compound as a yellow solid. ES MS m/z 500 (M−H).

### Step 5

### (cis and trans)-[4-(cyclobutylamino)cyclohexyl]({[3-({[(2,4,6-trimethylphenyl)amino]carbonyl}amino)-2-naphthalenyl]carbonyl}amino)acetic acid

NaB(OAc)₃H (0.057 g, 0.27 mmol) was added to a solution of (4-oxocyclohexyl)({[3-({[(2,4,6-trimethylphenyl)amino]carbonyl}amino)-2-naphthalenyl]carbonyl}amino)acetic acid (0.092 g, 0.18 mmol) and cyclobutylamine (0.12 g, 1.69 mmol) in DCE (5 mL). The solution was stirred for 16 h then ethyl acetate (100 mL) and water (50 mL) were added and the pH was adjusted to ca. 7 with 1N HCl. The organic layer was separated. The aqueous layer was acidified slightly (to ca. pH 3) with 1N HCl and extracted with ethyl acetate. The organic layer was dried over MgSO₄, filtered and concentrated to give 0.015 g (15%) of the title compound as a mixture of cis- and trans-isomers. APCI MS m/z 557 (M+H).

### Example 348

### (cis and trans)-[4-(4-morpholinyl)cyclohexyl]({[3-({[(2,4,6-trimethylphenyl)amino]carbonyl}amino)-2-naphthalenyl]carbonyl}amino)acetic acid

NaB(OAc)₃H (0.16 g, 0.78 mmol) was added to a solution of (4-oxocyclohexyl)({[3-({[(2,4,6-trimethylphenyl)amino]carbonyl}amino)-2-naphthalenyl]carbonyl}amino)acetic acid (0.13 g, 0.26 mmol) and morpholine (0.12 g, 1.38 mmol) in DCE (10 mL). The solution was stirred for 16 h then the solution was concentrated. MeOH (5 mL) and water (2 mL) were added. After stirring for 15 min, the mixture was concentrated to dryness and taken up in 3 mL of MeOH. The compound was purified by reverse phase HPLC (Gilson: MeCN, 1% TFA/water) to give 0.067 g (45%) of the title compound as a racemic mixture of cis- and trans-isomers. ES MS m/z 572 (M−H).

### Example 349

### (cis and trans)-methyl[4-({[(1,1-dimethylethyl)oxy]carbonyl}amino)cyclohexyl] ({[3-({[(2,4,6-trimethylphenyl)amino]carbonyl}amino)-2-naphthalenyl]carbonyl}amino)acetate

### Step 1

### methyl[4-({[(1,1-dimethylethyl)oxy]carbonyl}amino)cyclohexylidene]({[(phenylmethyl)oxy]carbonyl}amino)acetate

DBU (1.72 mL, 11.26 mmol) was added dropwise to a solution of methyl[bis(methyloxy)phosphoryl]({[(phenylmethyl)oxy]carbonyl}amino)acetate (3.11 g, 9.39 mmol) and 1,1-dimethylethyl (4-oxocyclohexyl)carbamate (2.00 g, 9.39 mmol) in DCM (25 mL). The solution was stirred at RT for 16 h then the solvent was removed on a rotary evaporator. Ethyl acetate (200 mL) and water (200 mL) were added followed by 1N HCl until the pH was acidic. The organic phase was separated, washed with water (100 mL), brine (100 mL), dried over MgSO₄, filtered and concentrated. Et₂O (150 mL) was added to the solid and the mixture was sonicated. The solid was filtered and dried to give 1.12 g (29%) of the title compound as a white powder.

### Step 2

### (cis and trans)-methyl amino[4-({[(1,1-dimethylethyl)oxy]carbonyl}amino)cyclohexyl]acetate

A mixture of methyl[4-({[(1,1-dimethylethyl)oxy]carbonyl}amino)cyclohexylidene]({[(phenylmethyl)oxy]carbonyl}amino)acetate (1.10 g, 2.63 mmol) and 10% Pd/C (0.15 g) in MeOH (75 mL) was stirred under hydrogen (60 psig) at RT for 24 h then carefully vented, filtered through Celite, and the solution was concentrated to give 0.80 g (106%) of the title compound as a racemic mixture of cis- and trans-isomers.

### Step 3

### (cis and trans)-methyl {[(3-amino-2-naphthalenyl)carbonyl]amino}[4-({[(1,1-dimethylethyl)oxy]carbonyl}amino)cyclohexyl]acetate

HATU (1.59 g, 4.19 mmol) was added to a suspension of 3-amino-2-naphthalenecarboxylic acid (0.52 g, 2.79 mmol), (cis and trans)-methyl amino[4-({[(1,1-dimethylethyl)oxy]carbonyl}amino)cyclohexyl]acetate (0.80 g, 2.79 mmol), and N,N-diisopropylethylamine (2.01 mL, 8.37 mmol) in DMF (15 mL) at RT. After 16 h, saturated NaHCO₃ solution (100 mL) and ethyl acetate (200 mL) were added. The organic layer was washed with brine (100 mL), dried over MgSO₄, filtered and concentrated. The crude product was purified on silica gel using an ISCO chromatography system (increasing gradient from 100% hexanes to 90% ethyl acetate/hexanes over 40 min) to give 0.96 g (76%) of the title compound as a yellow solid.

### Step 4

### (cis and trans)-methyl[4-({[(1,1-dimethylethyl)oxy]carbonyl}amino)cyclohexyl]({[3-({[(2,4,6-trimethylphenyl)amino]carbonyl}amino)-2-naphthalenyl]carbonyl}amino)acetate

A solution of (cis and trans)-methyl {[(3-amino-2-naphthalenyl)carbonyl]amino}[4-({[(1,1-dimethylethyl)oxy]carbonyl}amino)cyclohexyl]acetate (0.55 g, 1.21 mmol) and 2-isocyanato-1,3,5-trimethylbenzene (0.39 g, 2.41 mmol) in pyridine (10 mL) were stirred at RT for 72 h. The solvent was removed under reduced pressure and ethyl acetate (200 mL) and 1N HCl (200 mL) were added. The organic layer was washed with water (100 mL), brine (100 mL), dried over MgSO₄, filtered and concentrated to give 0.71 g (95%) of the title compound as a pale yellow solid. ES MS m/z 615 (M−H).

### Example 350

### (cis and trans)-[4-({[(1,1-dimethylethyl)oxy]carbonyl}amino)cyclohexyl]({[3-({[(2,4,6-trimethylphenyl)amino]carbonyl}amino)-2-naphthalenyl]carbonyl}amino)acetic acid

LiOH (0.12 g, 5.03 mmol) was dissolved in hot water (10 mL) and added while still hot to a solution of (cis and trans)-methyl[4-({[(1,1-dimethylethyl)oxy]carbonyl}amino)cyclohexyl]({[3-({[(2,4,6-trimethylphenyl)amino]carbonyl}amino)-2-naphthalenyl]carbonyl}amino)acetate (0.31 g, 0.50 mmol) in THF (5 mL) and MeOH (5 mL). The yellow solution was stirred for 3 h then ethyl acetate (300 mL) and 1N HCl solution (100 mL) were added. The organic layer was washed with brine (100 mL), dried over MgSO₄, filtered and concentrated to give 0.30 g (100%) of the title compound as a yellow solid. ES MS m/z 601 (M−H).

### Example 351

### (cis and trans)-(4-aminocyclohexyl)({[3-({[(2,4,6-trimethylphenyl)amino]carbonyl}amino)-2-naphthalenyl]carbonyl}amino)acetic acid trifluoroacetate

TFA (1.5 g, 13.2 mmol) was added to a solution of (cis and trans)-[4-({[(1,1-dimethylethyl)oxy]carbonyl}amino)cyclohexyl] ({[3-({[(2,4,6-trimethylphenyl)amino]carbonyl}amino)-2-naphthalenyl]carbonyl}amino)acetic acid (0.26 g, 0.43 mmol) in DCM (10 mL). The yellow solution was stirred for 16 h then concentrated to give a yellow oil (still has TFA remaining). The crude material was dissolved in DCM (5 mL) and Et₂O was added (50 mL). The solid was filtered, washed with Et₂O/hexane and dried under vacuum to give 0.26 g (98%) of the title compound as a white powder. ES MS m/z 501 (M−H).

### Example 352

### (cis and trans)-(4-{[(methylamino)carbonyl]amino}cyclohexyl)({[3-({[(2,4,6-trimethylphenyl)amino]carbonyl}amino)-2-naphthalenyl]carbonyl}amino)acetic acid

Methyl isocyanate (0.02 g, 0.35 mmol) was added to a solution of (cis and trans)-(4-aminocyclohexyl)({[3-({[(2,4,6-trimethylphenyl)amino]carbonyl}amino)-2-naphthalenyl]carbonyl}amino)acetic acid trifluoroacetate (0.125 g, 0.20 mmol) in pyridine (2 mL). After 2 h, LC/MS show ca. 1:1 mixture of starting material and product so more methyl isocyanate (0.025 g, 0.44 mmol) was added. The solution was stirred for 24 h then concentrated and ethyl acetate (100 mL) and 1N HCL (50 mL) were added. The organic layer was washed with brine, filtered to remove a terrible emulsion, dried over MgSO₄, filtered and concentrated. Reverse phase chromatography (Gilson: MeCN, 1% TFA/water) gave 0.026 g (23%) of the title compound as a white powder. ES MS m/z 558 (M−H).

### Example 353

### bis(1,1-dimethylethyl) N-{[3-({[(2,4,6-trimethylphenyl)amino]carbonyl}amino)-2-naphthalenyl]carbonyl}-L-aspartate

### Step 1

### bis(1,1-dimethylethyl) N-[(3-amino-2-naphthalenyl)carbonyl]-L-aspartate

HATU (3.24 g, 8.54 mmol) was added to a suspension of 3-amino-2-naphthalenecarboxylic acid (1.06 g, 5.69 mmol), bis(1,1-dimethylethyl) L-aspartate hydrochloride (1.60 g, 5.69 mmol), and N,N-diisopropylethylamine (2.05 mL, 8.54 mmol) in DMF (70 mL) at RT. After 16 h, saturated NaHCO₃ solution (100 mL) and ethyl acetate (300 mL) were added. The organic layer was washed with brine (100 mL), dried over MgSO₄, filtered and concentrated. The crude product was purified on silica gel using an ISCO chromatography system (increasing gradient from 100% hexanes to 90% ethyl acetate/hexanes over 20 min) to give 1.8 g (76%) of the title compound as a yellow solid.

### Step 2

### bis(1,1-dimethylethyl) N-{[3-({[(2,4,6-trimethylphenyl)amino]carbonyl}amino)-2-naphthalenyl]carbonyl}-L-aspartate

A solution of bis(1 μl-dimethylethyl) N-[(3-amino-2-naphthalenyl)carbonyl]-L-aspartate (0.41 g, 0.99 mmol) and 2-isocyanato-1,3,5-trimethylbenzene (0.32 g, 1.98 mmol) in pyridine (10 mL) were stirred at RT for 72 h. The solvent was removed under reduced pressure and ethyl acetate (200 mL) and water (200 mL) and 1N HCl (50 mL) were added. The organic layer was washed with water (100 mL), brine (100 mL), dried over MgSO₄, filtered and concentrated. The solid was purified on silica gel (ISCO: 10% ethyl acetate/hexanes to 100% ethyl acetate over 30 min) to give 0.55 g (97%) of the title compound as a pale orange solid. ES MS m/z 574 (M−H).

### Example 354

### N-{[3-({[(2,4,6-trimethylphenyl)amino]carbonyl}amino)-2-naphthalenyl]carbonyl}-L-aspartic acid

TFA (3 mL) was added to a solution of bis(1,1-dimethylethyl) N-{[3-({[(2,4,6-trimethylphenyl)amino]carbonyl}amino)-2-naphthalenyl]carbonyl}-L-aspartate (0.49 g, 0.85 mmol) in DCM (20 mL). The solution was occasionally heated to reflux with a heat gun then stirred overnight at RT. The solvent was removed under reduced pressure and DCM (5 mL) and Et₂O (60 mL) were added. The white solid was filtered and dried to give 0.26 g (66%) of the title compound as a white powder. ES MS m/z 462 (M−H).

### Example 355

### N-[(3-{[({2,6-dichloro-4-[(trifluoromethyl)oxy]phenyl}amino)carbonyl]amino}-2-naphthalenyl)carbonyl]-L-aspartic acid

### Step 1

### bis(1,1-dimethylethyl) N-[(3-{[({2,6-dichloro-4-[(trifluoromethyl)oxy]phenyl}amino)carbonyl]amino}-2-naphthalenyl)carbonyl]-L-aspartate

A solution of bis(1,1-dimethylethyl) N-[(3-amino-2-naphthalenyl)carbonyl]-L-aspartate (0.40 g, 0.97 mmol) and 1,3-dichloro-2-isocyanato-5-[(trifluoromethyl)oxy]benzene (0.52 g, 1.94 mmol) in pyridine (10 mL) was stirred at RT for 16 h. The solvent was removed under reduced pressure and ethyl acetate (100 mL) and water (50 mL) and 1N HCl (50 mL) were added. The organic layer was washed with water (100 mL), brine (100 mL), dried over MgSO₄, filtered and concentrated. The solid was purified on silica gel (ISCO: 10% ethyl acetate/hexanes to 100% ethyl acetate over 30 min) to give 0.43 g (65%) of the title compound as a yellow oil.

### Step 2

### N-[(3-{[({2,6-dichloro-4-[(trifluoromethyl)oxy]phenyl}amino)carbonyl]amino}-2-naphthalenyl)carbonyl]-L-aspartic acid

TFA (3 mL) was added to a solution of bis(1,1-dimethylethyl) N-[(3-{[({2,6-dichloro-4-[(trifluoromethyl)oxy]phenyl}amino)carbonyl]amino}-2-naphthalenyl)carbonyl]-L-aspartate (0.40 g, 0.58 mmol) in DCM (20 mL). The solution was heated occasionally to reflux with a heat gun then stirred at RT overnight. The solution was concentrated to give 0.33 g (99%) of the title compound as an off-white solid. ES MS m/z 572 (M−H).

### Example 356

### N-{[3-({[(2,4,6-trimethylphenyl)amino]carbonyl}amino)-2-naphthalenyl]carbonyl}-D-aspartic acid

### Step 1

### bis(phenylmethyl) N-[(3-amino-2-naphthalenyl)carbonyl]-D-aspartate

HATU (1.17 g, 3.09 mmol) was added to a suspension of 3-amino-2-naphthalenecarboxylic acid (0.38 g, 2.06 mmol), bis(phenylmethyl) D-aspartate tosylate (1.00 g, 2.06 mmol), and N,N-diisopropylethylamine (2.05 mL, 8.54 mmol) in DMF (40 mL) at RT. After 16 h, saturated NaHCO₃ solution (100 mL) and ethyl acetate (300 mL) were added. The organic layer was washed with brine (100 mL), dried over MgSO₄, filtered and concentrated. The crude product was purified on silica gel using an ISCO chromatography system (increasing gradient from 100% hexanes to 90% ethyl acetate/hexanes over 20 min) to give 0.77 g (77%) of the title compound as an orange oil.

### Step 2

### bis(phenylmethyl) N-{[3-({[(2,4,6-trimethylphenyl)amino]carbonyl}amino)-2-naphthalenyl]carbonyl}-D-aspartate

A solution of bis(phenylmethyl) N-[(3-amino-2-naphthalenyl)carbonyl]-D-aspartate (0.34 g, 0.97 mmol) and 2-isocyanato-1,3,5-trimethylbenzene (0.23 g, 1.40 mmol) in pyridine (5 mL) were stirred at RT for 5 h. The solvent was removed under reduced pressure and ethyl acetate (100 mL) and water (50 mL) and 1N HCl (50 mL) were added. The organic layer was washed with water (100 mL), brine (100 mL), dried over MgSO₄, filtered and concentrated. The solid was purified on silica gel (ISCO: 10% ethyl acetate/hexanes to 100% ethyl acetate over 30 min) to give 0.39 g (87%) of the title compound as an orange solid.

### Step 3

### N-{[3-({[(2,4,6-trimethylphenyl)amino]carbonyl}amino)-2-naphthalenyl]carbonyl}-D-aspartic acid

A solution of bis(phenylmethyl) N-{[3-({[(2,4,6-trimethylphenyl)amino]carbonyl}amino)-2-naphthalenyl]carbonyl}-D-aspartate (0.38 g, 0.59 mmol) and 10% Pd/C (0.15 g) in MeOH (25 mL) was stirred under hydrogen (60 psig) for 16 h. The reaction was carefully vented, filtered through Celite, and concentrated. The white residue was dissolved in hot ethyl acetate and hexane was added until a white solid formed. The solid was filtered and dried to give 0.071 g (26%) of the title compound as a white powder. ES MS m/z 462 (M−H).

### Example 357

### 1-{[3-({[(2,4,6-trimethylphenyl)amino]carbonyl}amino)-2-naphthalenyl]carbonyl}-D-proline

### Step 1

### 1,1-dimethylethyl 1-[(3-amino-2-naphthalenyl)carbonyl]-D-prolinate

HATU (1.83 g, 4.82 mmol) was added to a suspension of 3-amino-2-naphthalenecarboxylic acid (0.60 g, 3.21 mmol), 1,1-dimethylethyl D-prolinate (0.55 g, 3.21 mmol), and N,N-diisopropylethylamine (2.0 mL, 4.82 mmol) in DMF (15 mL) at RT. After 5 h, saturated NaHCO₃ solution (100 mL) and ethyl acetate (200 mL) were added. The organic layer was washed with brine (100 mL), dried over MgSO₄, filtered and concentrated. The crude product was purified on silica gel using an ISCO chromatography system (increasing gradient from 100% hexanes to 90% ethyl acetate/hexanes over 40 min) to give 0.88 g (81%) of the title compound as a white solid.

### Step 2

### 1,1-dimethylethyl 1-{[3-({[(2,4,6-trimethylphenyl)amino]carbonyl}amino)-2-naphthalenyl]carbonyl}-D-prolinate

A solution of 1,1-dimethylethyl 1-[(3-amino-2-naphthalenyl)carbonyl]-D-prolinate (0.40 g, 0.92 mmol) and 2-isocyanato-1,3,5-trimethylbenzene (0.38 g, 2.35 mmol) in pyridine (10 mL) were stirred at RT for 5 h. The solvent was removed under reduced pressure and ethyl acetate (100 mL) and 0.1 N HCl (100 mL) were added. The organic layer was washed with water (100 mL), brine (100 mL), dried over MgSO₄, filtered and concentrated to give a pale yellow oil. The product was purified on silica gel (ISCO: 5% ethyl acetate/hexanes to 100% ethyl acetate over 25 min) to give 0.50 g (85%) of the title compound as a white solid.

### Step 3

### 1-{[3-({[(2,4,6-trimethylphenyl)amino]carbonyl}amino)-2-naphthalenyl]carbonyl}-D-proline

TFA (3 mL) was added to a solution of 1,1-dimethylethyl 1-{[3-({[(2,4,6-trimethylphenyl)amino]carbonyl}amino)-2-naphthalenyl]carbonyl}-D-prolinate (0.48 g, 0.96 mmol) in DCM (20 mL). The solution was stirred overnight at RT then concentrated to give yellow solid. The crude material was dissolved in ethyl acetate (200 mL) and 1N NaOH added. The aqueous layer was separated, made acidic with 1N HCl, and extracted with ethyl acetate. The organic layer was dried over MgSO₄, filtered and concentrated to give 0.033 g of crude product. Reverse phase HPLC (Gilson: MeCN and 1% TFA/water) gave 0.026 g (6%) of the title compound as a white powder. ES MS m/z 444 (M−H).

### Example 358

### (2S)-[(1S)-3-oxocyclohexyl]({[3-({[(2,4,6-trimethylphenyl)amino]carbonyl}amino)-2-naphthalenyl]carbonyl}amino)ethanoic acid

### Step 1

### 1,1-dimethylethyl (2S)-[(diphenylmethylidene)amino][(1S)-3-oxocyclohexyl]ethanoate

A solution of 2-cyclohexen-1-one (1.04 g, 10.83 mmol) in DCM (10 mL) was added dropwise over ca. 10 min to a mixture of 1,1-dimethylethyl N-(diphenylmethylidene)glycinate (1.00 g, 3.39 mmol), Cinchonidine alkaloid (0.20 g, 0.34 mmol) and CsOH—H₂O (5.69 g, 33.9 mmol) in DCM (10 mL) at −78° C. After 2 h, the cold bath was removed and the solution was warmed to RT upon which the solution turned from yellow to dark brown. Ethyl acetate (200 mL) and water (200 mL) were added. The organic phase was washed with water (200 mL), brine (100 mL), dried over MgSO₄, filtered and concentrated to give 1.6 g of brown oil (¹H NMR shows product+alkaloid). The crude product was purified on silica gel (ISCO: 100% hexanes to 100% ethyl acetate over 30 min) to give 1.17 g (88%) of the title compound as a ca. 4:1 mixture of diastereomers.

### Step 2

### 1,1-dimethylethyl (2S)-amino[(1S)-3-oxocyclohexyl]ethanoate

A mixture of 1,1-dimethylethyl (2S)-[(diphenylmethylidene)amino][(1S)-3-oxocyclohexyl]ethanoate (1.10 g, 2.81 mmol) and 10% Pd/C (0.15 g) in MeOH (25 mL) was stirred under hydrogen (60 psig) for 16 h, then carefully vented, filtered through Celite and concentrated. ¹H NMR showed a new product but some aromatic protons still remained. Therefore the material was dissolved in MeOH and 10% Pd/C and 2 drops of concentrated HCl were added. The mixture was stirred under hydrogen (60 psig) for 4 h then carefully vented, filtered through Celite and concentrated to give 0.61 g of crude product. The material was used without further purification.

### Step 3

### 1,1-dimethylethyl (2S)-{[(3-amino-2-naphthalenyl)carbonyl]amino}[(1S)-3-oxocyclohexyl]ethanoate

HATU (1.52 g, 4.01 mmol) was added to a suspension of 3-amino-2-naphthalenecarboxylic acid (0.50 g, 2.67 mmol), 1,1-dimethylethyl (2S)-amino[(1S)-3-oxocyclohexyl]ethanoate (0.61 g, 2.67 mmol), and N,N-diisopropylethylamine (1.9 mL, 8.01 mmol) in DMF (10 mL) at RT. After 5 h, saturated NaHCO₃ solution (100 mL) and ethyl acetate (200 mL) were added. The organic layer was washed with brine (100 mL), dried over MgSO₄, filtered and concentrated. The crude product was partially purified on silica gel using an ISCO chromatography system (increasing gradient from 100% hexanes to 90% ethyl acetate/hexanes over 40 min) to give 0.37 g (35%) of the title compound as a yellow solid in ca. 60% purity.

### Step 4

### 1,1-dimethylethyl (2S)-[(1S)-3-oxocyclohexyl]({[3-({[(2,4,6-trimethylphenyl)amino]carbonyl}amino)-2-naphthalenyl]carbonyl}amino)ethanoate

A solution of 1,1-dimethylethyl (2S)-{[(3-amino-2-naphthalenyl)carbonyl]amino}[(1S)-3-oxocyclohexyl]ethanoate (0.45 g, 1.14 mmol) and 2-isocyanato-1,3,5-trimethylbenzene (0.22 g, 1.36 mmol) in pyridine (5 mL) were stirred at RT for 5 h. The solvent was removed under reduced pressure and ethyl acetate (100 mL) and 0.1N HCl (100 mL) were added. The organic layer was washed with water (100 mL), brine (100 mL), dried over MgSO₄, filtered and concentrated. The crude material was purified on silica gel (ISCO chromatography: 100% hexanes to 80% ethyl acetate/hexanes) to give 0.18 g (28%) of the title compound as a yellow solid.

### Step 5

### (2S)-[(1S)-3-oxocyclohexyl]({[3-({[(2,4,6-trimethylphenyl)amino]carbonyl}amino)-2-naphthalenyl]carbonyl}amino)ethanoic acid

TFA (3 mL) was added to a solution of 1,1-dimethylethyl (2S)-[(1S)-3-oxocyclohexyl]({[3-({[(2,4,6-trimethylphenyl)amino]carbonyl}amino)-2-naphthalenyl]carbonyl}amino)ethanoate (0.17 g, 0.30 mmol) in DCM (20 mL). The solution was heated occasionally to reflux with a heat gun then stirred at RT overnight. The solution was concentrated to give an off-white solid. The residue was dissolved in a small amount of DCM & MeOH then Et₂O was added followed by hexane. The solid was filtered and dried to give 0.041 g (27%) of the title compound as a white powder. ES MS m/z 500 (M−H).

### Example 359

### (2S)-[(1S)-3-hydroxycyclohexyl]({[3-({[(2,4,6-trimethylphenyl)amino]carbonyl}amino)-2-naphthalenyl]carbonyl}amino)ethanoic acid; and

### Example 360

### (2S)-{(1S)-3-[(trifluoroacetyl)oxy]cyclohexyl}({[3-({[(2,4,6-trimethylphenyl)amino]carbonyl}amino)-2-naphthalenyl]carbonyl}amino)ethanoic acid

### Step 1

### 1,1-dimethylethyl (2S)-[(diphenylmethylidene)amino][(1S)-3-oxocyclohexyl]ethanoate

A solution of 2-cyclohexen-1-one (2.58 g, 26.84 mmol) in DCM (20 mL) was added dropwise over ca. 10 min to a mixture of 1,1-dimethylethyl N-(diphenylmethylidene)glycinate (2.44 g, 8.26 mmol), Cinchonidine alkaloid (0.50 g, 0.82 mmol) and CsOH—H₂O (13.15 g, 78.32 mmol) in DCM (20 mL) at −780° C. The cold bath was allowed to warm to RT then ethyl acetate (200 mL) and water (200 mL) were added. The organic phase was washed with water (200 mL), brine (100 mL), dried over MgSO₄, filtered and concentrated. The crude product was purified on silica gel (ISCO: 100% hexanes to 100% ethyl acetate over 30 min) to give 1.79 g (55%) of the title compound as a clear oil.

### Step 2

### 1,1-dimethylethyl (2S)-[(diphenylmethyl)amino][(1S)-3-hydroxycyclohexyl]ethanoate

NaBH₄ (0.13 g, 3.54 mmol) was added to a solution of 1,1-dimethylethyl (2S)-[(diphenylmethylidene)amino][(1S)-3-oxocyclohexyl]ethanoate (0.63 g, 1.61 mmol) in MeOH (10 mL) at RT. The solution was stirred overnight then water (100 mL) and ethyl acetate (200 mL) added. Organic layer washed with brine, dried over MgSO₄, filtered and concentrated to give 0.63 g (99%) of the title compound as a clear oil.

### Step 3

### 1,1-dimethylethyl (2S)-amino[(1S)-3-hydroxycyclohexyl]ethanoate

A mixture of 1,1-dimethylethyl (2S)-[(diphenylmethyl)amino][(1S)-3-hydroxycyclohexyl]ethanoate (0.63 g, 1.59 mmol) and 10% Pd/C (0.10 g) in MeOH (25 mL) was stirred under hydrogen (60 psig) at RT for 16 h. The reaction was carefully vented, diluted with ethyl acetate, filtered through Celite and concentrated to give the title compound which was used without further purification.

### Step 4

### 1,1-dimethylethyl (2S)-{[(3-amino-2-naphthalenyl)carbonyl]amino}[(1S,3R)-3-hydroxycyclohexyl]ethanoate

HATU (1.52 g, 4.01 mmol) was added to a suspension of 3-amino-2-naphthalenecarboxylic acid (0.50 g, 2.67 mmol), 1,1-dimethylethyl (2S)-amino[(1S)-3-hydroxycyclohexyl]ethanoate (0.61 g, 2.67 mmol), and N,N-diisopropylethylamine (1.9 mL, 8.01 mmol) in DMF (10 mL) at RT. After 5 h, saturated NaHCO₃ solution (100 mL) and ethyl acetate (200 mL) were added. The organic layer was washed with brine (100 mL), dried over MgSO₄, filtered and concentrated. The crude product was purified on silica gel using an ISCO chromatography system (increasing gradient from 100% hexanes to 90% ethyl acetate/hexanes over 40 min) to give 0.37 g (34%) of the title compound as a yellow solid.

### Step 5

### (2S)-[(1S)-3-hydroxycyclohexyl]({[3-({[(2,4,6-trimethylphenyl)amino]carbonyl}amino)-2-naphthalenyl]carbonyl}amino)ethanoic acid and

### (2S)-{(1S)-3-[(trifluoroacetyl)oxy]cyclohexyl}({[3-({[(2,4,6-trimethylphenyl)amino]carbonyl}amino)-2-naphthalenyl]carbonyl}amino)ethanoic acid

A solution of 1,1-dimethylethyl (2S)-{[(3-amino-2-naphthalenyl)carbonyl]amino}[(1S,3R)-3-hydroxycyclohexyl]ethanoate (0.37 g, 0.93 mmol) and 2-isocyanato-1,3,5-trimethylbenzene (0.15 g, 0.93 mmol) in pyridine (5 mL) were stirred at RT for 16 h, then concentrated to dryness. 1N HCl (50 mL) and ethyl acetate (50 mL) were added. The organic layer was washed with brine, dried over MgSO₄, filtered and concentrated. The crude material was dissolved in DCM (10 mL) and TFA (3 mL) was added. The yellow solution was occasionally heated to reflux and stirred until no starting material remained. The solution was concentrated to dryness and dried under vacuum. Reverse-phase HPLC (Gilson: MeCN, 1% TFA/water) gave two products:

0.034 g (7% over two steps) of (2S)-[(1S)-3-hydroxycyclohexyl]({[3-({[(2,4,6-trimethylphenyl)amino]carbonyl}amino)-2-naphthalenyl]carbonyl}amino)ethanoic acid. ES MS m/z 502 (M−H). and

0.060 g (11% over two steps) of. (2S)-{(1S)-3-[(trifluoroacetyl)oxy]cyclohexyl}({[3-({[(2,4,6-trimethylphenyl)amino]carbonyl}amino)-2-naphthalenyl]carbonyl}amino)ethanoic acid. ES MS m/z 598 (M−H).

### Example 361

### bis(1 μl-dimethylethyl) N-{[4′-(methyloxy)-3-({[(2,4,6-trimethylphenyl)amino]carbonyl}amino)-4-biphenylyl]carbonyl}-L-aspartate

### Step 1

### 3-amino-4′-(methyloxy)-4-biphenylcarboxylic acid

A solution of 4′-(methyloxy)-3-nitro-4-biphenylcarboxylic acid (0.50 g, 1.83 mmol) and 10% Pd/C (100 mg) under H₂ (60 psig) in EtOH (20 mL) was stirred at RT for 16 h (a cloudy suspension formed). The suspension was dissolved in ethyl acetate (50 mL), filtered through Celite, and concentrated to give 0.43 g (98%) of the title compound as an orange solid.

### Step 2

### bis(1 μl-dimethylethyl) N-{[3-amino-4′-(methyloxy)-4-biphenylyl]carbonyl}-L-aspartate

HATU (0.40 g, 1.04 mmol) was added to a solution of 3-amino-4′-(methyloxy)-4-biphenylcarboxylic acid (0.23 g, 0.95 mmol), bis(1,1-dimethylethyl) L-aspartate hydrochloride (0.53 g, 1.89 mmol), and N,N-diisopropylethylamine (2 mL, 8.34 mmol) in DCM (10 mL) at RT. After 3 h, saturated NaHCO₃ solution (100 mL) and ethyl acetate (200 mL) were added. The organic layer was washed with saturated NaHCO₃ solution, brine, dried over MgSO₄, filtered and concentrated. The crude oil was purified on silica gel (hexanes to 90% ethylacetate/hexanes over 30 min) to give 0.63 g (71%) of the title compound as a white powder.

### Step 3

### bis(1,1-dimethylethyl) N-{[4′-(methyloxy)-3-({[(2,4,6-trimethylphenyl)amino]carbonyl}amino)-4-biphenylyl]carbonyl}-L-aspartate

A solution of bis(1,1-dimethylethyl) N-{[3-amino-4′-(methyloxy)-4-biphenylyl]carbonyl}-L-aspartate (0.32 g, 0.67 mmol) and 2-isocyanato-1,3,5-trimethylbenzene (0.54 g, 3.35 mmol) in pyridine (3 mL) were stirred at RT for 4 h then the pyridine was removed in vacuo. The residue was taken up in 1N HCl (50 mL), water (200 mL), and ethyl acetate (200 mL). The organic layer was washed with water, brine, dried over MgSO₄, filtered and concentrated. The crude product was purified on silica gel (ISCO: 100% hexanes to 90% ethyl acetate/hexanes over 25 min) to give 0.40 g (94%) of the title compound as a white powder. APCI MS m/z 630 (M−H).

### Example 362

### N-{[4′-(methyloxy)-3-({[(2,4,6-trimethylphenyl)amino]carbonyl}amino)-4-biphenylyl]carbonyl}-L-aspartic acid

TFA (3 mL) was added to a solution of bis(1,1-dimethylethyl) N-{[4′-(methyloxy)-3-({[(2,4,6-trimethylphenyl)amino]carbonyl}amino)-4-biphenylyl]carbonyl}-L-aspartate (0.40 g, 0.63) in DCM (10 mL). The solution was heated occasionally to reflux with a heat gun then stirred at RT for 2 days. The solution was concentrated and the resulting oil was dissolved in DCM (1 mL) and Et₂O (5 mL) then hexanes (5 mL) were added. The white precipitate was filtered and dried to give 0.18 g (55%) of the title compound as a white powder. ES MS m/z 518 (M−H).

### Example 363

### (2S)-4-(ethyloxy)-4-oxo-2-({[3-({[(2,4,6-trimethylphenyl)amino]carbonyl}amino)-2-naphthalenyl]carbonyl}amino)butanoic acid

### Step 1

### 4-ethyl 1-(phenylmethyl) N-{[(1,1-dimethylethyl)oxy]carbonyl}-L-aspartate

DMAP (0.30 g, 2.47 mmol) was added to a solution of (3S)-3-({[(1,1-dimethylethyl)oxy]carbonyl}amino)-4-oxo-4-[(phenylmethyl)oxy]butanoic acid (4.00 g, 12.37 mmol) and EDC (1.78 g, 9.28 mmol) in EtOH (14.11 g, 37.12 mmol) and DCM (40 mL) at RT. The solution was stirred for 2 days then sat NaHCO₃ solution and ethyl acetate were added. The organic layer was washed with sat NaHCO₃ solution, water, brine, dried MgSO₄, filtered and concentrated to give 4.41 g (101%) of the title compound as a colorless oil.

### Step 2

### 4-ethyl 1-(phenylmethyl) L-aspartate trifluoroacetate

TFA (10 mL) was added to a solution of 4-ethyl 1-(phenylmethyl) N-{[(1,1-dimethylethyl)oxy]carbonyl}-L-aspartate (4.41 g, 12.51 mmol) in DCM (20 mL). The yellow solution was stirred overnight then concentrated to give 5.2 g of the title compound as a yellow oil (note: excess TFA still present).

### Step 3

### 4-ethyl 1-(phenylmethyl) N-[(3-amino-2-naphthalenyl)carbonyl]-L-aspartate

HATU (1.63 g, 4.29 mmol) was added to a solution of 3-amino-2-naphthalenecarboxylic acid (0.54 g, 2.86 mmol), 4-ethyl 1-(phenylmethyl) L-aspartate trifluoroacetate (1.57 g, 4.30 mmol) and N,N-diisopropylethylamine (3.43 mL, 14.3 mmol) in DMF (10 mL) at RT under nitrogen. After 1 h, saturated NaHCO₃ soln (100 mL) and ethyl acetate (200 mL) were added. The organic layer was washed with brine, dried over MgSO₄, filtered and concentrated. The crude oil was purified on silica gel (ISCO: 100% hexanes to 90% ethyl acetate/hexanes over 30 minutes) to give 1.11 g (92%) of the title compound as an orange oil.

### Step 4

### 4-ethyl 1-(phenylmethyl) N-{[3-({[(2,4,6-trimethylphenyl)amino]carbonyl}amino)-2-naphthalenyl]carbonyl}-L-aspartate

A solution of 4-ethyl 1-(phenylmethyl) N-[(3-amino-2-naphthalenyl)carbonyl]-L-aspartate (0.61 g, 1.45 mmol) and 2-isocyanato-1,3,5-trimethylbenzene (0.70 g, 4.35 mmol) in pyridine (4 mL) were stirred at RT for 2 days. The pyridine was removed in vacuo and ethyl acetate (200 mL) and sat NaHCO₃ solution (150 mL) were added. The organic layer was washed with water, brine, dried over MgSO₄, filtered and concentrated to give 0.41 g (49%) of the title compound as a white powder.

### Step 5

### (2S)-4-(ethyloxy)-4-oxo-2-({[3-({[(2,4,6-trimethylphenyl)amino]carbonyl}amino)-2-naphthalenyl]carbonyl}amino)butanoic acid

A solution of 4-ethyl 1-(phenylmethyl) N-{[3-({[(2,4,6-trimethylphenyl)amino]carbonyl}amino)-2-naphthalenyl]carbonyl}-L-aspartate (0.20 g, 0.34 mmol) in EtOH (15 mL) and ethyl acetate (2 mL) was purged with nitrogen. Next, 10% Pd/C (0.10 g) was added and the suspension was stirred under one atmosphere of hydrogen (balloon) for 16 h. The next day TLC showed a little starting material remaining so the H₂ balloon was reinserted and stirred for another 3 h. The reaction was carefully vented and filtered through Celite. The solution was concentrated to give 0.14 g (83%) of the title compound as a white solid. ES MS m/z 490 (M−H).

### Example 364

### N-{[2-({[(2,6-dichlorophenyl)amino]carbonyl}amino)-4-fluorophenyl]carbonyl}-L-aspartic acid

### Step 1

### bis(1,1-dimethylethyl) N-[(4-fluoro-2-nitrophenyl)carbonyl]-L-aspartate

HATU (7.71 g, 20.29 mmol) was added to a suspension of 4-fluoro-2-nitrobenzoic acid (2.50 g, 13.51 mmol), bis(1,1-dimethylethyl) L-aspartate hydrochloride (6.08 g, 21.62 mmol), and N,N-diisopropylethylamine (9.7 mL, 40.53 mmol) in DCM (40 mL) at RT. After 2 days, 1N HCl (100 mL), water (100 mL) and ethyl acetate (400 mL) were added. The organic layer was washed with saturated NaHCO₃ solution (300 mL), brine (100 mL), dried over MgSO₄, filtered and concentrated to give 3.78 g (68%) of the title compound as an orange oil.

### Step 2

### bis(1,1-dimethylethyl) N-[(2-amino-4-fluorophenyl)carbonyl]aspartate hydrochloride

A mixture of bis(1,1-dimethylethyl) N-[(4-fluoro-2-nitrophenyl)carbonyl]-L-aspartate (3.78 g, 9.12 mmol) and 10% Pd/C (0.2 g) in EtOH (10 mL) were stirred under hydrogen (60 psig) for 3 h, then the reaction vessel was carefully vented. Ethyl acetate (50 mL) was added and the mixture was filtered through Celite and concentrated to give 3.31 g of yellow oil. ¹H NMR shows that the reduction was not complete. Therefore the material was dissolved in EtOH (10 mL) and 10% Pd/C (0.2 g) was added. The mixture was stirred under hydrogen (60 psig) for 5 h then carefully vented. Ethyl acetate (50 mL) was added and the mixture was filtered through Celite and concentrated. The yellow oil was dissolved in DCM (10 mL) and Et₂O (20 mL) then 1N HCl in Et₂O (10 mL) was added. The solvent was removed by rotary evaporation to give 2.65 g (69%) of the title compound as a pale orange solid.

### Step 3

### bis(1 μl-dimethylethyl) N-{[2-({[(2,6-dichlorophenyl)amino]carbonyl}amino)-4-fluorophenyl]carbonyl}-L-aspartate

A solution of bis(1,1-dimethylethyl) N-[(2-amino-4-fluorophenyl)carbonyl]aspartate hydrochloride (0.40 g, 0.96 mmol) and 1,3-dichloro-2-isocyanatobenzene (0.75 g, 3.99 mmol) in pyridine (4 mL) were stirred at RT for 16 h then the pyridine was removed in vacuo. The residue was taken up in 1N HCl (50 mL), water (200 mL), and ethyl acetate (200 mL). The organic layer was washed with water, brine, dried over MgSO₄, filtered and concentrated. The crude product was purified on silica gel (ISCO: 100% hexanes to 90% ethyl acetate/hexanes) to give 0.37 g (68%) of the title compound as a white powder.

### Step 4

### N-{[2-({[(2,6-dichlorophenyl)amino]carbonyl}amino)-4-fluorophenyl]carbonyl}-L-aspartic acid

TFA (5 mL) was added to a solution of bis(1,1-dimethylethyl) N-{[2-({[(2,6-dichlorophenyl)amino]carbonyl}amino)-4-fluorophenyl]carbonyl}-L-aspartate (0.30 g, 0.53) in DCM (10 mL). The solution was heated occasionally to reflux with a heat gun then stirred at RT for 24 h. The solution was concentrated to give 0.20 g (82%) of the title compound as a white powder. ES MS m/z 456 (M−H).

### Example 365

### N-{[4-fluoro-2-({[(2,4,6-trimethylphenyl)amino]carbonyl}amino)phenyl]carbonyl}-L-aspartic acid

### Step 1

### bis(1,1-dimethylethyl) N-{[4-fluoro-2-({[(2,4,6-trimethylphenyl)amino]carbonyl}amino)phenyl]carbonyl}-L-aspartate

A solution of bis(1,1-dimethylethyl) N-[(2-amino-4-fluorophenyl)carbonyl]-L-aspartate hydrochloride (0.40 g, 0.96 mmol) and 2-isocyanato-1,3,5-trimethylbenzene (0.77 g, 4.78 mmol) in pyridine (4 mL) were stirred at RT for 16 h then the pyridine was removed in vacuo. The residue was taken up in 1N HCl (50 mL), water (200 mL), and ethyl acetate (200 mL). The organic layer was washed with water, brine, dried over MgSO₄, filtered and concentrated. The crude product was purified on silica gel (ISCO: 100% hexanes to 90% ethyl acetate/hexanes) to give 0.36 g (69%) of the title compound as a white powder.

### Step 2

### N-{[4-fluoro-2-({[(2,4,6-trimethylphenyl)amino]carbonyl}amino)phenyl]carbonyl}-L-aspartic acid

TFA (5 mL) was added to a solution of bis(1,1-dimethylethyl) N-{[4-fluoro-2-({[(2,4,6-trimethylphenyl)amino]carbonyl}amino)phenyl]carbonyl}-L-aspartate (0.31 g, 0.57 mmol) in DCM (10 mL). The solution was heated occasionally to reflux with a heat gun then stirred at RT overnight. The solution was concentrated to give an oil which was dissolved in DCM (1 mL) and Et₂O (5 mL) then hexanes (5 mL) was added. The white precipitate was filtered and dried to give 0.16 g (55%) of the title compound as a white powder. ES MS m/z 430 (M−H).

### Example 366

### 4-ethyl 1-(phenylmethyl)N-{[4′-(methyloxy)-3-({[(2,4,6-trimethylphenyl)amino]carbonyl}amino)-4-biphenylyl]carbonyl}-L-aspartate

### Step 1

### methyl 4′-(methyloxy)-3-nitro-4-biphenylcarboxylate

Two microwave reaction vials were each charged with a mixture of methyl 4-chloro-2-nitrobenzoate (1.00 g, 4.64 mmol), [4-(methyloxy)phenyl]boronic acid (0.78 g, 5.10 mmol), cesium fluoride (2.11 g, 13.92 mmol) and Pd(Cy₃)₂Cl₂ (0.17 g, 0.23 mmol) in MeCN (10 mL) and water (4 mL). Each vial was heated to 150° C. for 5 min in a microwave reactor then the two reaction mixtures were combined in a separatory funnel, diluted with ethyl acetate (300 mL), washed with water (200 mL), brine (200 mL), dried over MgSO₄, filtered and concentrated. The crude oil was purified on silica gel (ISCO: eluting with 100% hexanes to 100% ethyl acetate over 50 min) to give 2.21 g (83%) of the title compound as a yellow oil.

### Step 2

### 4′-(methyloxy)-3-nitro-4-biphenylcarboxylic acid

A solution of LiOH (0.92 g, 38.33 mmol) in hot water (15 mL) was added to a solution of methyl 4′-(methyloxy)-3-nitro-4-biphenylcarboxylate (2.20 g, 7.67 mmol) in THF (15 mL) and MeOH (5 mL). The yellow solution was stirred at RT for 5 h then 1N HCl (100 mL) and ethyl acetate (250 mL) were added. The organic layer was separated, washed with brine, dried over MgSO₄, filtered and concentrated to give 2.02 g (96%) of the title compound as a pale yellow solid.

### Step 3

### 3-amino-4′-(methyloxy)-4-biphenylcarboxylic acid

A solution of 4′-(methyloxy)-3-nitro-4-biphenylcarboxylic acid (0.71 g, 2.60 mmol) and 10% Pd/C (0.15 g) under hydrogen (60 psig) in MeOH (20 mL) was stirred at RT for 16 h (a cloudy suspension formed). The suspension was dissolved in ethyl acetate (50 mL), filtered through Celite, and concentrated to give 0.63 g (100%) of the title compound as a yellow solid.

### Step 4

### 4-ethyl 1-(phenylmethyl) N-{[3-amino-4′-(methyloxy)-4-biphenylyl]carbonyl}-L-aspartate

HATU (1.48 g, 3.89 mmol) was added to a solution of 3-amino-4′-(methyloxy)-4-biphenylcarboxylic acid (0.63 g, 2.59 mmol), 4-ethyl 1-(phenylmethyl) L-aspartate trifluoroacetate (1.89 g, 5.19 mmol) and N,N-diisopropylethylamine (3 mL, 12.5 mmol) in DCM (10 mL) at RT. After 3 h, saturated NaHCO₃ solution (100 mL) and ethyl acetate (200 mL) were added. The organic layer was washed with saturated NaHCO₃ solution, brine, dried over MgSO₄, filtered and concentrated. The crude oil was purified on silica gel (100% hexanes to 90% ethyl acetate/hexanes over 30 min) to give 0.66 g (53%) of the title compound as a yellow oil.

### Step 5

### 4-ethyl 1-(phenylmethyl) N-{[4′-(methyloxy)-3-({[(2,4,6-trimethylphenyl)amino]carbonyl}amino)-4-biphenylyl]carbonyl}-L-aspartate

A solution of 4-ethyl 1-(phenylmethyl) N-{[3-amino-4′-(methyloxy)-4-biphenylyl]carbonyl}-L-aspartate (0.19 g, 0.40 mmol) and 2-isocyanato-1,3,5-trimethylbenzene (0.19 g, 1.20 mmol) in pyridine (3 mL) were stirred at RT for 4 h then the pyridine was removed in vacuo. The residue was taken up in 1N HCl (25 mL), water (100 mL), and ethyl acetate (100 mL). The organic layer was washed with water, brine, dried over MgSO₄, filtered and concentrated to give 0.23 g (90%) of the title compound as a yellow solid. ES MS m/z 638 (M+H).

### Example 367

### (2S)-4-(ethyloxy)-2-({[4′-(methyloxy)-3-({[(2,4,6-trimethylphenyl)amino]carbonyl}amino)-4-biphenylyl]carbonyl}amino)-4-oxobutanoic acid

A solution of 4-ethyl 1-(phenylmethyl) N-{[4′-(methyloxy)-3-({[(2,4,6-trimethylphenyl)amino]carbonyl}amino)-4-biphenylyl]carbonyl}-L-aspartate (0.21 g, 0.36 mmol) in EtOH (15 mL) and ethyl aceate (15 mL) was purged with nitrogen. Next, 10% Pd/C (0.10 g) was added and the suspension was stirred under one atmosphere of hydrogen (balloon) for 16 h. The next day, TLC showed that a little starting material still remained so the hydrogen balloon was re-inserted and stirred for another 3 h. The reaction was carefully vented and filtered through Celite. The solvent was removed to give 0.11 g (56%) of the title compound as a white solid. ES MS m/z 546 (M−H).

### Example 368

### 4-ethyl 1-(phenylmethyl) N-{[3-({[(4-bromo-2,6-dimethylphenyl)amino]carbonyl}amino)-4′-(methyloxy)-4-biphenylyl]carbonyl}-L-aspartate

A solution of 4-ethyl 1-(phenylmethyl) N-{[3-amino-4′-(methyloxy)-4-biphenylyl]carbonyl}-L-aspartate (0.46 g, 0.97 mmol) and 5-bromo-2-isocyanato-1,3-dimethylbenzene (0.66 g, 2.90 mmol) in pyridine (3 mL) were stirred at RT for 16 h then the pyridine was removed in vacuo. The residue was taken up in 1N HCl (25 mL), water (100 mL), and ethyl acetate (100 mL). The organic layer was washed with water, brine, dried over MgSO₄, filtered and concentrated to give 0.62 g (91%) of the title compound as a yellow powder. ES MS m/z 703 (M+H).

### Example 369

### (2S)-2-({[3-({[(2,6-dimethyl-4-propylphenyl)amino]carbonyl}amino)-4′-(methyloxy)-4-biphenylyl]carbonyl}amino)-4-(ethyloxy)-4-oxobutanoic acid

### Step 1

### 4-ethyl 1-(phenylmethyl) N-{[3-[({[2,6-dimethyl-4-(2-propen-1-yl)phenyl]amino}carbonyl)amino]-4′-(methyloxy)-4-biphenylyl]carbonyl}-L-aspartate

A mixture of 4-ethyl 1-(phenylmethyl) N-{[3-({[(4-bromo-2,6-dimethylphenyl)amino]carbonyl}amino)-4′-(methyloxy)-4-biphenylyl]carbonyl}-L-aspartate (0.58 g, 0.83 mmol), tributyl(2-propen-1-yl)stannane (0.30 g, 0.91 mmol) and Pd(PPh₃)₄ (0.057 g, 0.05 mmol) in MeCN (10 mL) were heated in a Microwave Reactor at 150° C. for 30 min. The mixture was extracted between ethyl acetate and water. The organic layer was washed with brine, dried over MgSO₄, filtered and concentrated. The crude material was purified on silica gel (ISCO: 100% hexanes to 80% ethyl acetate/hexanes) to give 0.39 g (72%) of the title compound as a white solid.

### Step 2

### (2S)-2-({[3-({[(2,6-dimethyl-4-propylphenyl)amino]carbonyl}amino)-4′-(methyloxy)-4-biphenylyl]carbonyl}amino)-4-(ethyloxy)-4-oxobutanoic acid

A solution of 4-ethyl 1-(phenylmethyl) N-{[3-[({[2,6-dimethyl-4-(2-propen-1-yl)phenyl]amino}carbonyl)amino]-4′-(methyloxy)-4-biphenylyl]carbonyl}-L-aspartate (0.38 g, 0.57 mmol) in EtOH (15 mL) and ethyl acetate (15 mL) was purged with nitrogen. Next, 10% Pd/C (0.10 g) was added and the suspension was stirred under one atmosphere of hydrogen (balloon) for 16 h. TLC showed a little starting material remaining so the hydrogen balloon was re-inserted. After stirring for 16 h, the flask was carefully vented and the mixture was filtered through Celite and concentrated. SFC purification gave 0.16 g (49%) of the title compound as a white powder. APCI m/z 574 (M−H).

### Example 370

### N-{[3-({[(2,6-dimethyl-4-propylphenyl)amino]carbonyl}amino)-4′-(methyloxy)-4-biphenylyl]carbonyl}-L-aspartic acid

LiOH (0.080 g, 3.33 mmol) was dissolved in hot water (5 mL) and added while still warm to a solution of (2S)-2-({[3-({[(2,6-dimethyl-4-propylphenyl)amino]carbonyl}amino)-4′-(methyloxy)-4-biphenylyl]carbonyl}amino)-4-(ethyloxy)-4-oxobutanoic acid (0.06 g, 0.10 mmol) in THF (5 mL) and MeOH (5 mL). The solution was stirred at RT for 6 h then 1N HCl was added until the pH<7. Ethyl acetate (100 mL) and water (50 mL) were added and the organic layer was washed with brine (50 mL), dried over MgSO₄, filtered and concentrated to give 0.045 g (79%) of the title compound as a white powder. APCI MS m/z 547 (M−H).

### Example 371

### 4-(1,1-dimethylethyl) 1-methyl N-{[4′-(methyloxy)-3-({[(2,4,6-trimethylphenyl)amino]carbonyl}amino)-4-biphenylyl]carbonyl}-L-aspartate

### Step 1

### 4-(1,1-dimethylethyl) 1-methyl N-{[4′-(methyloxy)-3-nitro-4-biphenylyl]carbonyl}-L-aspartate

HATU (0.94 g, 2.47 mmol) was added to a solution of 4′-(methyloxy)-3-nitro-4-biphenylcarboxylic acid (0.45 g, 1.65 mmol) and N,N-diisopropylethylamine (1 mL, 4.17 mmol) in DCM (10 mL) at RT. After 5 min, 4-(1,1-dimethylethyl) 1-methyl L-aspartate hydrochloride (0.48 g, 2.14 mmol) was added. The yellow solution was stirred at RT for 2 h then saturated NaHCO₃ solution (100 mL) and ethyl acetate (200 mL) were added. The organic layer was washed with saturated NaHCO₃ solution, brine, dried over MgSO₄, filtered and concentrated. The crude solid was purified on silica gel (100% hexanes to 90% ethyl acetate/hexanes over 30 min) to give 0.66 g (87%) of the title compound as a white solid.

### Step 2

### 4-(1,1-dimethylethyl) 1-methyl N-{[3-amino-4′-(methyloxy)-4-biphenylyl]carbonyl}-L-aspartate

A mixture of 4-(1,1-dimethylethyl) 1-methyl N-{[4′-(methyloxy)-3-nitro-4-biphenylyl]carbonyl}-L-aspartate (0.66 g, 1.44 mmol) and 10% Pd/C (0.10 g) under hydrogen (60 psig) in MeOH (15 mL) and ethyl acetate (15 mL) was stirred at RT for 16 h. Ethyl acetate (50 mL) was added and the mixture was filtered through Celite. The solution was concentrated to give 0.62 g (100%) of the title compound as a white solid.

### Step 3

### 4-(1,1-dimethylethyl) 1-methyl N-{[4′-(methyloxy)-3-({[(2,4,6-trimethylphenyl)amino]carbonyl}amino)-4-biphenylyl]carbonyl}-L-aspartate

A solution of 4-(1,1-dimethylethyl) 1-methyl N-{[3-amino-4′-(methyloxy)-4-biphenylyl]carbonyl}-L-aspartate (0.62 g, 1.45 mmol) and 2-isocyanato-1,3,5-trimethylbenzene (0.70 g, 4.35 mmol) in pyridine (3 mL) was stirred at RT for 5 h then the pyridine was removed in vacuo. The residue was taken up in 1N HCl (25 mL), water (200 mL), and ethyl acetate (200 mL). The organic layer was washed with water, brine, dried over MgSO₄, filtered and concentrated to give 0.81 g (95%) of the title compound as a yellow powder. ES MS m/z 588 (M−H).

### Example 372

### (3S)-4-(methyloxy)-3-({[4′-(methyloxy)-3-({[(2,4,6-trimethylphenyl)amino]carbonyl}amino)-4-biphenylyl]carbonyl}amino)-4-oxobutanoic acid

A solution of 4-(1,1-dimethylethyl) 1-methyl N-{[4′-(methyloxy)-3-({[(2,4,6-trimethylphenyl)amino]carbonyl}amino)-4-biphenylyl]carbonyl}-L-aspartate (0.79 g, 1.34 mmol) and TFA (4 mL) in DCM (5 mL) was stirred at RT for 16 h then concentrated to dryness. The residue was taken up in DCM (ca. 5 mL) and Et₂O (10 mL) and hexanes (30 mL) were added. The solid was filtered and dried under vacuum to give 0.64 g (90%) of the title compound as an off-white solid. ES MS m/z 532 (M−H).

### Example 373

### N-{[3′,4′-difluoro-3-({[(2,4,6-trimethylphenyl)amino]carbonyl}amino)-4-biphenylyl]carbonyl}-O-(1,1-dimethylethyl)-L-threonine

### Step 1

### methyl 3′,4′-difluoro-3-nitro-4-biphenylcarboxylate

Five separate microwave reaction vials were each charged with a mixture of methyl 4-chloro-2-nitrobenzoate (1.00 g, 5.64 mmol), (3,4-difluorophenyl)boronic acid (0.81 g, 5.10 mmol), Pd(Cy₃)₂Cl₂ (0.17 g, 0.23 mmol) and CsF (2.11 g, 13.90 mmol) in MeCN (10 mL) and water (5 mL). The vials were sealed then heated to 1500° C. for 7 min. The vials were vented, diluted with ethyl acetate and reaction mixtures were combined. The solids were filtered off and the solution was washed with water, dried over Na₂SO₄, filtered and concentrated to give 5.67 g (83%) of the title compound.

### Step 2

### 3′,4′-difluoro-3-nitro-4-biphenylcarboxylic acid

LiOH (1.39 g, 57.95 mmol) was dissolved in hot water (30 mL) and added while still warm to a solution of methyl 3′,4′-difluoro-3-nitro-4-biphenylcarboxylate (5.66 g, 19.32 mmol) in THF (100 mL) and MeOH (30 mL). The solution was stirred for 16 h then the reaction was concentrated to dryness. Water (50 mL) was added, followed by 1N HCl until the pH<7. The white solid was filtered then dissolved in ethyl acetate (150 mL), dried over MgSO₄, filtered and concentrated to give 5.25 g (97%) of the title compound as a white powder.

### Step 3

### methyl N-[(3′,4′-difluoro-3-nitro-4-biphenylyl)carbonyl]-O-(1,1-dimethylethyl)-L-threoninate

N,N-diisopropylethylamine (1.0 mL, 4.2 mmol) was added to a suspension of 3′,4′-difluoro-3-nitro-4-biphenylcarboxylic acid (0.50 g, 1.79 mmol), methyl O-(1,1-dimethylethyl)-L-threoninate hydrochloride (0.49 g, 2.15 mmol) and HATU (1.02 g, 2.69 mmol) in DCM (10 mL) and DMF (2 mL). The yellow solution was stirred for 1 h then sat NaHCO₃ solution (150 mL) and ethyl acetate (200 mL) were added. The organic layer was washed with sat NaHCO₃ solution (150 mL), brine (150 mL), dried over MgSO₄, filtered and concentrated. The crude material was purified on silica gel (ISCO: 100% hexanes to 80% ethyl acetate/hexanes over 20 min) to give 0.79 g (98%) of the title compound as a white solid.

### Step 4

### methyl N-[(3-amino-3′,4′-difluoro-4-biphenylyl)carbonyl]-O-(1,1-dimethylethyl)-L-threoninate

A solution of methyl N-[(3′,4′-difluoro-3-nitro-4-biphenylyl)carbonyl]-O-(1,1-dimethylethyl)-L-threoninate (0.78 g, 1.73 mmol) and 10% Pd/C (0.10 g) under hydrogen (60 psig) in MeOH (15 mL) and ethyl acetate (15 mL) was stirred at RT for 5 h. The flask was carefully vented and ethyl acetate (50 mL) was added. The mixture was filtered through Celite and concentrated to give 0.71 g (97%) of the title compound as an off-white solid.

### Step 5

### methyl N-{[3′,4′-difluoro-3-({[(2,4,6-trimethylphenyl)amino]carbonyl}amino)-4-biphenylyl]carbonyl}-O-(1,1-dimethylethyl)-L-threoninate

A solution of methyl N-[(3-amino-3′,4′-difluoro-4-biphenylyl)carbonyl]-O-(1,1-dimethylethyl)-L-threoninate (0.20 g, 0.48 mmol) and 2-isocyanato-1,3,5-trimethylbenzene (0.09 g, 0.57 mmol) in pyridine (3 mL) were stirred at RT for 16 h. The solvent was removed under reduced pressure and ethyl acetate (100 mL) and 0.1 N HCl (100 mL) were added. The organic layer was washed with water (100 mL), brine (100 mL), dried over MgSO₄, filtered and concentrated. The crude material was purified on silica gel (ISCO: 100% hexanes to 80% ethyl acetate/hexanes over 30 min) to give 0.25 g (90%) of the title compound as a white solid.

### Step 6

### N-{[3′,4′-difluoro-3-({[(2,4,6-trimethylphenyl)amino]carbonyl}amino)-4-biphenylyl]carbonyl}-O-(1,1-dimethylethyl)-L-threonine

LiOH (0.031 g, 1.29 mmol) was dissolved in hot water (5 mL) and added while still warm to a solution of methyl N-{[3′,4′-difluoro-3-({[(2,4,6-trimethylphenyl)amino]carbonyl}amino)-4-biphenylyl]carbonyl}-O-(1,1-dimethylethyl)-L-threoninate (0.25 g, 0.43 mmol) in THF (5 mL) and MeOH (5 mL). After 4 h, the reaction was concentrated to dryness and water (5 mL) was added, followed by 1N HCl until the pH<7. Ethyl acetate (100 mL) was added and the organic layer was washed with brine (50 mL), dried over MgSO₄, filtered and concentrated to give 0.21 g (86%) of the title compound as a white powder. ES MS m/z 566 (M−H).

### Example 374

### (2S)-2-({[3′,4′-difluoro-3-({[(2,4,6-trimethylphenyl)amino]carbonyl}amino)-4-biphenylyl]carbonyl}amino)-4-(ethyloxy)-4-oxobutanoic acid

### Step 1

### 4-ethyl 1-(phenylmethyl) N-[(3′,4′-difluoro-3-nitro-4-biphenylyl)carbonyl]-L-aspartate

N,N-diisopropylethylamine (1.00 mL, 4.17 mmol) was added to a suspension of 3′,4′-difluoro-3-nitro-4-biphenylcarboxylic acid (0.45 g, 1.61 mmol) and HATU (0.92 g, 2.42 mmol) in DCM (10 mL) and DMF (2 mL). After 5 min, 4-ethyl 1-(phenylmethyl) L-aspartate trifluoroacetate (1.18 g, 3.23 mmol) was added and the yellow solution was stirred for 16 h then sat NaHCO₃ solution (150 mL) and ethyl acetate (200 mL) were added. The organic layer was washed with sat NaHCO₃ solution (150 mL), brine (150 mL), dried over MgSO₄, filtered and concentrated. The crude material was purified on silica gel (ISCO: 100% hexanes to 80% ethyl acetate/hexanes over 20 min) to give 0.47 g (57%) of the title compound as a white solid.

### Step 2

### 4-ethyl 1-(phenylmethyl) N-[(3-amino-3′,4′-difluoro-4-biphenylyl)carbonyl]-L-aspartate

A mixture of 4-ethyl 1-(phenylmethyl) N-[(3′,4′-difluoro-3-nitro-4-biphenylyl)carbonyl]-L-aspartate (0.47 g, 0.92 mmol) and Pt on sulfided carbon (5 wt. %, 0.10 g) in MeOH (15 mL) were stirred under one atmosphere of hydrogen (balloon) at RT. After 4 h, the balloon was removed and ethyl acetate (100 mL) was added. The mixture was filtered through Celite and the solution was concentrated to give a yellow oil which was dissolved in Et₂O (25 mL) then filtered through Celite again. The solution was concentrated to give 0.44 g (99%) of the title compound as a yellow oil.

### Step 3

### 4-ethyl 1-(phenylmethyl) N-{[3′,4′-difluoro-3-({[(2,4,6-trimethylphenyl)amino]carbonyl}amino)-4-biphenylyl]carbonyl}-L-aspartate

A solution of 4-ethyl 1-(phenylmethyl) N-[(3-amino-3′,4′-difluoro-4-biphenylyl)carbonyl]-L-aspartate (0.44 g, 0.91 mmol) and 2-isocyanato-1,3,5-trimethylbenzene (0.29 g, 1.82 mmol) in pyridine (6 mL) were stirred at RT for 16 h. The solvent was removed under reduced pressure and ethyl acetate (200 mL) and 1N HCl (50 mL) and water (150 mL) were added. The organic layer was washed with saturated NaHCO₃ solution (100 mL), brine (100 mL), dried over MgSO₄, filtered and concentrated. The crude material was purified on silica gel (ISCO: 100% hexanes to 80% ethyl acetate/hexanes over 30 min) to give 0.44 g (75%) of the title compound as a white solid.

### Step 4

### (2S)-2-({[3′,4′-difluoro-3-({[(2,4,6-trimethylphenyl)amino]carbonyl}amino)-4-biphenylyl]carbonyl}amino)-4-(ethyloxy)-4-oxobutanoic acid

A solution of 4-ethyl 1-(phenylmethyl) N-{[3′,4′-difluoro-3-({[(2,4,6-trimethylphenyl)amino]carbonyl}amino)-4-biphenylyl]carbonyl}-L-aspartate (0.44 g, 0.68 mmol) in MeOH (15 mL) and ethyl acetate (5 mL) was purged with nitrogen. Next, 10% Pd/C (0.10 g) was added and the suspension was stirred under one atmosphere of hydrogen (balloon) for 16 h then carefully vented and filtered through Celite. The solvent was removed and the resulting orange solid was partially dissolved in hot ethyl acetate (10 mL), sonicated and allowed to cool to RT. The solid was filtered and dried to give 0.21 g (56%) of title compound as a white powder. APCI m/z 554.29 (M+H).

### Example 375

### N-{[3′,4′-difluoro-3-({[(2,4,6-trimethylphenyl)amino]carbonyl}amino)-4-biphenylyl]carbonyl}-L-aspartic acid

LiOH (0.041 g, 1.70 mmol) was dissolved in hot water (5 mL) and added while still warm to a solution of (2S)-2-({[3′,4′-difluoro-3-({[(2,4,6-trimethylphenyl)amino]carbonyl}amino)-4-biphenylyl]carbonyl}amino)-4-(ethyloxy)-4-oxobutanoic acid (0.092 g, 0.17 mmol) in THF (5 mL) and MeOH (5 mL). The solution was stirred at RT for 6 h then 1N HCl was added until the pH<7. Ethyl acetate (100 mL) and water (50 mL) were added and the organic layer was washed with brine (50 mL), dried over MgSO₄, filtered and concentrated to give 0.056 g (64%) of the title compound as a white powder. APCI MS m/z 524.33 (M−H).

### Example 376

### N-{[3′,4′-difluoro-3-({[(2,4,6-trimethylphenyl)amino]carbonyl}amino)-4-biphenylyl]carbonyl}-D-aspartic acid

### Step 1

### bis(phenylmethyl) N-[(3′,4′-difluoro-3-nitro-4-biphenylyl)carbonyl]-D-aspartate

Bis(phenylmethyl) D-aspartate 4-methylbenzenesulfonate (0.67 g, 1.38 mmol) was added to a suspension of HATU (0.61 g, 1.59 mmol), 3′,4′-difluoro-3-nitro-4-biphenylcarboxylic acid (0.30 g, 1.06 mmol) and N,N-diisopropylethylamine (0.76 mL, 3.17 mmol) in DCM (10 mL) and DMF (5 mL) at RT. After 16 h, saturated NaHCO₃ solution (100 mL) and ethyl acetate (200 mL) were added. The organic layer was washed with brine (100 mL), dried over MgSO₄, filtered and concentrated. The crude product was purified on silica gel using an ISCO chromatography system (increasing gradient from 100% hexanes to 90% ethyl acetate/hexanes over 20 min) to give 0.41 g (67%) of the title compound as a yellow solid.

### Step 2

### bis(phenylmethyl) N-[(3-amino-3′,4′-difluoro-4-biphenylyl)carbonyl]-D-aspartate

A mixture of bis(phenylmethyl) N-[(3′,4′-difluoro-3-nitro-4-biphenylyl)carbonyl]-D-aspartate (0.41 g, 0.71 mmol) and Pt on sulfided carbon (5 wt. %, 0.12 g) in MeOH (20 mL) under one atmosphere of hydrogen (balloon) was stirred at RT for 6 h. The balloon was removed, ethyl acetate (100 mL) was added and the mixture was filtered through Celite and concentrated. The brown oil was purified on silica gel using an ISCO chromatography system (gradient: 100% hexanes to 100% ethyl acetate over 25 minutes) to give 0.36 g (94%) of the title compound as a yellow oil.

### Step 3

### bis(phenylmethyl) N-{[3′,4′-difluoro-3-({[(2,4,6-trimethylphenyl)amino]carbonyl}amino)-4-biphenylyl]carbonyl}-D-aspartate

A solution of bis(phenylmethyl) N-[(3-amino-3′,4′-difluoro-4-biphenylyl)carbonyl]-D-aspartate (0.36 g, 0.66 mmol) and 2-isocyanato-1,3,5-trimethylbenzene (0.21 g, 1.32 mmol) in pyridine (6 mL) were stirred at RT for 72 h. The solvent was removed under reduced pressure and ethyl acetate (200 mL) and 1N HCl (50 mL) and water (150 mL) were added. The organic layer was washed with saturated NaHCO₃ solution (100 mL), brine (100 mL), dried over MgSO₄, filtered and concentrated. The crude material was dissolved in hot ethyl acetate (ca. 5 mL) and hexanes added until cloudy. The precipitate was filtered and dried to give 0.27 g (58%) of the title compound as a yellow solid.

### Step 4

### N-{[3′,4′-difluoro-3-({[(2,4,6-trimethylphenyl)amino]carbonyl}amino)-4-biphenylyl]carbonyl}-D-aspartic acid

A solution of bis(phenylmethyl) N-{[3′,4′-difluoro-3-({[(2,4,6-trimethylphenyl)amino]carbonyl}amino)-4-biphenylyl]carbonyl}-D-aspartate (0.27 g, 0.38 mmol) in MeOH (15 mL) was purged with nitrogen. Next, 10% Pd/C (0.10 g) was added and the suspension was stirred under one atmosphere of hydrogen (balloon) for 16 h then carefully vented and filtered through Celite. The solvent was removed under vacuum and the resulting material was dissolved in hot ethyl acetate (ca. 5 mL) and triturated with Et₂O and hexanes. The resulting solid was filtered and dried to give 0.12 g (60%) of the title compound as a white powder. APCI m/z 524 (M+H).

### Example 377

### methyl N²-{[4′-(methyloxy)-3-({[(2,4,6-trimethylphenyl)amino]carbonyl}amino)-4-biphenylyl]carbonyl}-L-asparaginate

A 30% aqueous solution of ammonium hydroxide (1 mL) was added to a suspension of (3S)-4-(methyloxy)-3-({[4′-(methyloxy)-3-({[(2,4,6-trimethylphenyl)amino]carbonyl}amino)-4-biphenylyl]carbonyl}amino)-4-oxobutanoic acid (0.13 g, 0.24 mmol) and HATU (0.14 g, 0.37 mmol) in DCM (5 mL). After stirring at RT for 3 h, the reaction was concentrated to dryness. Water (5 mL) was added and the resulting white precipitate was filtered and dried under vacuum to give 0.088 g (68%) of title compound as a white powder. APCI MS m/z 531 (M−H).

### Example 378

### N²-{[4′-(methyloxy)-3-({[(2,4,6-trimethylphenyl)amino]carbonyl}amino)-4-biphenylyl]carbonyl}-L-asparagine

LiOH (0.10 g, 4.17 mmol) was dissolved in hot water (3 mL) and added while still warm to a solution of methyl N²-{[4′-(methyloxy)-3-({[(2,4,6-trimethylphenyl)amino]carbonyl}amino)-4-biphenylyl]carbonyl}-L-asparaginate (0.060 g, 0.11 mmol) in THF (3 mL) and MeOH (3 mL). The solution was stirred at RT for 5 h then 1N HCl was added until the pH<7. The resulting white powder was filtered and dried. The solid was sonicated with MeOH, filtered, washed with Et₂O and dried under vacuum to give 0.020 g (35%) of the title compound as a white powder. APCI MS m/z 517 (M−H).

### Example 379

### N-{[3′,4′-difluoro-3-({[(2,4,6-trimethylphenyl)amino]carbonyl}amino)-4-biphenylyl]carbonyl}-L-glutamic acid

### Step 1

### bis(phenylmethyl) N-[(3′,4′-difluoro-3-nitro-4-biphenylyl)carbonyl]-L-glutamate

N,N-diisopropylethylamine (1.00 mL, 4.17 mmol) was added to a suspension of 3′,4′-difluoro-3-nitro-4-biphenylcarboxylic acid (0.30 g, 1.08 mmol) and HATU (0.61 g, 1.61 mmol) in DCM (10 mL) and DMF (2 mL). After 5 min, bis(phenylmethyl) L-glutamate 4-methylbenzenesulfonate (0.805 g, 1.61 mmol) was added and the yellow solution was stirred for 16 h at RT then 1N HCl solution (50 mL) and ethyl acetate (100 mL) were added. The organic layer was washed with saturated NaHCO₃ solution (100 mL), brine (100 mL), dried over MgSO₄, filtered and concentrated. The crude material was purified on silica gel (ISCO: 100% hexanes to 80% ethyl acetate/hexanes over 20 min) to give 0.53 g (84%) of the title compound as a clear oil.

### Step 2

### bis(phenylmethyl) N-[(3-amino-3′,4′-difluoro-4-biphenylyl)carbonyl]-L-glutamate

A mixture of bis(phenylmethyl) N-[(3′,4′-difluoro-3-nitro-4-biphenylyl)carbonyl]-L-glutamate (0.50 g, 0.85 mmol) and Pt on sulfided carbon (5 wt. %, 0.12 g) in MeOH (20 mL) was stirred under one atmosphere of hydrogen gas (balloon) at RT. After 4 h, the balloon was removed, ethyl acetate (100 mL) was added and the mixture was filtered through Celite then concentrated. The yellow oil was purified on silica gel using an ISCO chromatography system (gradient: 100% hexanes to 100% ethyl acetate over 25 minutes) to give a yellow solid that was a mixture of two compounds. The material was dissolved in hot ethyl acetate and hexane was slowly added until a precipitate just began to form, then the solution was allowed to cool overnight. The solid was filtered off and the resulting filtrate was concentrated to give 0.15 g (32%) of the title compound as a yellow solid.

### Step 3

### bis(phenylmethyl) N-{[3′,4′-difluoro-3-({[(2,4,6-trimethylphenyl)amino]carbonyl}amino)-4-biphenylyl]carbonyl}-L-glutamate

A solution of bis(phenylmethyl) N-[(3-amino-3′,4′-difluoro-4-biphenylyl)carbonyl]-L-glutamate (0.15 g, 0.27 mmol) and 2-isocyanato-1,3,5-trimethylbenzene (0.086 g, 0.54 mmol) in pyridine (2 mL) were stirred at RT for 16 h. The solvent was removed under reduced pressure and ethyl acetate (150 mL) and 1N HCl (50 mL) and water (100 mL) were added. The organic layer was washed with saturated NaHCO₃ solution (100 mL), brine (100 mL), dried over MgSO₄, filtered and concentrated. The crude material was dissolved in a minimal amount of MeOH/DCM (ca. 1 mL, 2 mL), then Et₂O (5 mL) and hexanes (5 mL) were added. The solid was filtered and dried under vacuum to give 0.090 (47%) of the title compound as a yellow solid.

### Step 4

### N-{[3′,4′-difluoro-3-({[(2,4,6-trimethylphenyl)amino]carbonyl}amino)-4-biphenylyl]carbonyl}-L-glutamic acid

A solution of bis(phenylmethyl) N-{[3′,4′-difluoro-3-({[(2,4,6-trimethylphenyl)amino]carbonyl}amino)-4-biphenylyl]carbonyl}-L-glutamate (0.09 g, 0.13 mmol) in MeOH (15 mL) and ethyl acetate (5 mL) was purged with nitrogen. Next, 10% Pd/C (0.06 g) was added and the suspension was stirred under one atmosphere of hydrogen (balloon) for 16 h then carefully vented and filtered through Celite. The solvent was removed to give 0.058 g (87%) of the title compound as a white powder. APCI m/z 538 (M−H).

### Example 380

### 4′-(methyloxy)-3-({[(2,4,6-trimethylphenyl)amino]carbonyl}amino)-4-biphenylcarboxylic acid

A solution of 3-amino-4′-(methyloxy)-4-biphenylcarboxylic acid (0.15 g, 0.62 mmol) and 2-isocyanato-1,3,5-trimethylbenzene (0.21 g, 0.93 mmol) were stirred in pyridine (5 mL) overnight. The next day, TLC showed some starting material still remaining so more 2-isocyanato-1,3,5-trimethylbenzene (ca. 0.2 g, 1.24 mmol) was added. When the starting material was gone as evident by TLC, 1N HCl was added followed by ethyl acetate (50 mL). The organic layer was washed with brine, dried over MgSO₄, filtered and concentrated to give a white solid. The crude material was purified on silica gel to give 0.015 g (6%) of the title compound. ES m/z 403 (M−H).

### Example 381

### 3-{[({2,6-dichloro-4-[(trifluoromethyl)oxy]phenyl}amino)carbonyl]amino}-4′-(methyloxy)-4-biphenylcarboxylic acid

A solution of 3-amino-4′-(methyloxy)-4-biphenylcarboxylic acid (0.15 g, 0.62 mmol) and 1,3-dichloro-2-isocyanato-5-[(trifluoromethyl)oxy]benzene (0.25 g, 0.93 mmol) were stirred in DCM (5 mL) and N,N-diisopropylethylamine (1 mL, 4.17 mmol)) at RT overnight. Next, 1N HCl was added followed by ethyl acetate (50 mL). The organic layer was washed with brine, dried over MgSO₄, filtered and concentrated to give a white solid. Recrystallization from hot ethyl acetate gave 0.030 g (9%) of the title compound. APCI m/z 617 (M+H).

### Example 382

### 2-({[(2,6-dichlorophenyl)amino]carbonyl}amino)-5-phenyl-3-thiophenecarboxylic acid

### Step 1

### 1,1-dimethylethyl 2-amino-5-phenyl-3-thiophenecarboxylate

To a mixture of phenylacetaldehyde 4.0 g (0.0333 mole) and 1,1-dimethylethyl cyanoacetate 4.69 g (0.0333 mole) and sulfur 1.17 g (0.0366 mole) in ethanol (50 mL) was added morpholine 3.33 mL (0.038 mole) and the contents heated under nitrogen at 50° C. for 18 h. After filtration, water was added to the reaction to precipitate the desired product. Filtration of the solid followed by washing with 30% aqueous ethanol and drying afforded 6.4 g (70%) of a yellow solid.

### Step 2

### 1,1-dimethylethyl 2-({[(2,6-dichlorophenyl)amino]carbonyl}amino)-5-phenyl-3-thiophenecarboxylate

To 1,1-dimethylethyl 2-amino-5-phenyl-3-thiophenecarboxylate 0.5 g (1.818 mmole) and 1,3-dichloro-2-isocyanatobenzene 0.342 g (1.818 mmole) was added DMF (3.0 mL), triethylamine 0.255 mL (1.818 mmole), and the contents heated at 80° C. for 2 h. The crude reaction was loaded onto an isco column and eluted with a gradient of EtOAc/Hexane (0-60%) over 35 min to afford 0.53 g (63%) of a white solid.

### Step 3

### 2-({[(2,6-dichlorophenyl)amino]carbonyl}amino)-5-phenyl-3-thiophenecarboxylic acid

To 1,1-dimethylethyl 2-({[(2,6-dichlorophenyl)amino]carbonyl}amino)-5-phenyl-3-thiophenecarboxylate 0.1 g (0.216 mmol) was added TFA 0.324 mL (4.32 mmol) and the contents heated at 50° C. for 3 h. Concentration of the reaction under vacuum afforded the desired product. ES MS m/z 407 (M+H).

### Example 383

### methyl(2S)-cyclohexyl({[2-({[(2,6-dichlorophenyl)amino]carbonyl}amino)-5-phenyl-3-thienyl]carbonyl}amino)ethanoate

To 2-({[(2,6-dichlorophenyl)amino]carbonyl}amino)-5-phenyl-3-thiophenecarboxylic acid 0.1 g (0.246 mmol) in DMF (3.0 mL) was added HATU 0.094 g (0.246 mmol) and Hunig's base (0.419 mmol), followed by the addition of methyl(2S)-amino(cyclohexyl)ethanoate hydrochloride 0.051 g (0.246 mmol) and the contents stirred at r.t. for 16 h. The crude reaction mixture was loaded onto an isco column and eluted with a gradient of EtOAc/Hexane (0-60%) over 35 min to afford 0.060 g (44%) of a colorless oil. ES MS m/z 560 (M+H).

### Example 384

### (2S)-cyclohexyl({[2-({[(2,6-dichlorophenyl)amino]carbonyl}amino)-5-phenyl-3-thienyl]carbonyl}amino)ethanoic acid

To methyl(2S)-cyclohexyl({[2-({[(2,6-dichlorophenyl)amino]carbonyl}amino)-5-phenyl-3-thienyl]carbonyl}amino)ethanoate 0.055 g (0.098 mmol) was added a 1.0 M solution of lithium hydroxide 0.108 mL (0.108 mmol) and the contents stirred for 16 h. The reaction was then acidified to pH=4.0, the precipitated product was then extracted with EtOAc. Drying of the organic layer with magnesium sulfate followed by concentration under vacuum afforded the desired product 0.030 g (57%) as a yellow solid. ES MS m/z 555 (M+H).

### Example 385

### 3-({[(2,6-dichlorophenyl)amino]carbonyl}amino)-5-(4-fluorophenyl)-2-thiophenecarboxylic acid

### Step 1

### 3-amino-5-(4-fluorophenyl)-2-thiophenecarboxylic acid

To methyl 3-amino-5-(4-fluorophenyl)-2-thiophenecarboxylate 3.0 g (0.0119 mole) in dioxane (40 mL) was added a 1 M solution of lithium hydroxide 14.3 mL (14.34 mmol) and the contents refluxed for 4 h. Acidification of the reaction mixture with 1N HCl to pH=4 gave a solid which was filtered and dried under vacuum.

### Step 2

### 3-({[(2,6-dichlorophenyl)amino]carbonyl}amino)-5-(4-fluorophenyl)-2-thiophenecarboxylic acid

To a solution of 3-amino-5-(4-fluorophenyl)-2-thiophenecarboxylic acid 0.5 g (2.10 mmol) in DMF (3 mL) was added 1,3-dichloro-2-isocyanatobenzene 0.395 g (2.10 mmol) and triethylamine and the contents heated to 500° C. for 16 h. After addition of sat. Na₂CO₃ (20 mL), water and EtOAc were added and the layers separated. The aqueous solution was acidified and then extracted with EtOAc and dried over magnesium sulfate followed by concentration under vacuum to afford the desired product as an off white solid 0.64 g (71%). ES MS m/z 425 (M+H).

### Example 386

### methyl(2S)-cyclohexyl({[3-({[(2,6-dichlorophenyl)amino]carbonyl}amino)-5-(4-fluorophenyl)-2-thienyl]carbonyl}amino)ethanoate

To 3-({[(2,6-dichlorophenyl)amino]carbonyl}amino)-5-(4-fluorophenyl)-2-thiophenecarboxylic acid 0.090 g (0.212 mmol) in DMF (2.0 mL) was added HATU 0.088 g (0.233 mmol) and Hunig's base 0.075 mL (0.424 mmol), followed by the addition of methyl(2S)-amino(cyclohexyl)ethanoate hydrochloride 0.044 g (0.212 mmol) and the contents stirred at r.t. for 16 h. The crude reaction mixture was loaded onto an isco column and eluted with a gradient of EtOAc/Hexane (0-60%) over 35 min to afford 0.100 g (82%) of a colorless oil. ES MS m/z 578 (M+H).

### Example 387

### (2S)-cyclohexyl({[3-({[(2,6-dichlorophenyl)amino]carbonyl}amino)-5-(4-fluorophenyl)-2-thienyl]carbonyl}amino)ethanoic acid

To methyl(2S)-cyclohexyl({[3-({[(2,6-dichlorophenyl)amino]carbonyl}amino)-5-(4-fluorophenyl)-2-thienyl]carbonyl}amino)ethanoate 0.090 g (0.156 mmol) was added a 1.0 M solution of lithium hydroxide 0.187 mL (0.187 mmol) and the contents stirred for 16 h. The reaction was then acidified to pH=4.0, the precipitated product was then extracted with EtOAc. Drying of the organic layer with magnesium sulfate followed by concentration under vacuum afforded to desired product 0.76 g (87%) as a yellow solid. (U22007-21-2). ES MS m/z 564 (M+H).

### Example 388

### 2-({[(2,6-dichlorophenyl)amino]carbonyl}amino)-5-methyl-3-thiophenecarboxylic acid

### Step 1

### 1,1-dimethylethyl 2-amino-5-methyl-3-thiophenecarboxylate

To a mixture of propanal 2.41 g (0.0333 mole) and 1,1-dimethylethyl cyanoacetate 4.69 g (0.0333 mole) and sulfur 1.17 g (0.0366 mole) in ethanol (50 mL) was added morpholine 3.33 mL (0.038 mole) and the contents heated under nitrogen at 500° C. for 18 h. After filtration, the contents were concentrated and loaded onto an isco column eluting with EtOAc/Hexane (0-100%) to afford 2.1 g (24%) of the desired product.

### Step 2

### 1,1-dimethylethyl 2-({[(2,6-dichlorophenyl)amino]carbonyl}amino)-5-methyl-3-thiophenecarboxylate

To 1,1-dimethylethyl 2-amino-5-methyl-3-thiophenecarboxylate 0.5 g (2.53 mmole) and 1,3-dichloro-2-isocyanatobenzene 0.475 g (2.53 mmole) was added DMF (4.0 mL) and triethylamine 0.354 mL (2.53 mmole) and the contents heated at 80° C. for 2 h. The crude reaction was loaded onto an isco column and eluted with a gradient of EtOAc/Hexane (0-60%) over 35 min to afford 0.54 g (53%) of a yellow solid.

### Step 3

### 2-({[(2,6-dichlorophenyl)amino]carbonyl}amino)-5-methyl-3-thiophenecarboxylic acid

To 1,1-dimethylethyl 2-({[(2,6-dichlorophenyl)amino]carbonyl}amino)-5-methyl-3-thiophenecarboxylate 0.150 g (0.375 mmol) was added TFA 0.144 mL (1.87 mmol) and dioxane (3.0 mL) and the contents heated at 50° C. for 3 h. Concentration of the reaction under vacuum afforded the desired product. ES MS m/z 345 (M+H).

### Example 389

### methyl(2S)-cyclohexyl({[2-({[(2,6-dichlorophenyl)amino]carbonyl}amino)-5-methyl-3-thienyl]carbonyl}amino)ethanoate

To 2-({[(2,6-dichlorophenyl)amino]carbonyl}amino)-5-methyl-3-thiophenecarboxylic acid 0.129 g (0.375 mmol) in DMF (2.0 mL) was added HATU 0.142 g (0.375 mmol) and Hunig's base 0.067 mL (0.375 mmol), followed by the addition of methyl(2S)-amino(cyclohexyl)ethanoate hydrochloride 0.078 g (0.375 mmol) and the contents stirred at r.t. for 16 h. The crude reaction mixture was loaded onto an isco column and eluted with a gradient of EtOAc/Hexane (0-60%) over 35 min to afford 0.11 g (59%) of a colorless oil. ES MS m/z 498 (M+H).

### Example 390

### (2S)-cyclohexyl({f[2-({[(2,6-dichlorophenyl)amino]carbonyl}amino)-5-methyl-3-thienyl]carbonyl}amino)ethanoic acid

To methyl (2S)-cyclohexyl({[2-({[(2,6-dichlorophenyl)amino]carbonyl}amino)-5-methyl-3-thienyl]carbonyl}amino)ethanoate 0.053 g (0.107 mmol) was added a 1.0 M solution of lithium hydroxide 0.127 mL (0.128 mmol) and the contents stirred for 16 h. The reaction was then acidified to pH=4.0, the precipitated product was then extracted with EtOAc. Drying of the organic layer with magnesium sulfate followed by concentration under vacuum afforded to desired product 0.042 g (%) as a yellow solid. ES MS m/z 484 (M+H).

### Example 391

### 5-phenyl-2-({[(2,4,6-trichlorophenyl)amino]carbonyl}amino)-3-thiophenecarboxylic acid

### Step 1

### 1,1-dimethylethyl 5-phenyl-2-({[(2,4,6-trichlorophenyl)amino]carbonyl}amino)-3-thiophenecarboxylate

To 1,1-dimethylethyl 2-amino-5-phenyl-3-thiophenecarboxylate 0.5 g (1.818 mmole) and 1,3,5-trichloro-2-isocyanatobenzene 0.398 g (1.818 mmole) was added DMF (3.0 mL) and triethylamine 0.255 mL (1.818 mmole) and the contents heated at 80° C. for 2 h. The crude reaction was loaded onto an isco column and eluted with a gradient of EtOAc/Hexane (0-60%) over 35 min to afford 0.560 g (70%) of a white solid.

### Step 2

### 5-phenyl-2-({[(2,4,6-trichlorophenyl)amino]carbonyl}amino)-3-thiophenecarboxylic acid

To 1,1-dimethylethyl 5-phenyl-2-({[(2,4,6-trichlorophenyl)amino]carbonyl}amino)-3-thiophenecarboxylate 0.150 g (0.301 mmol) was added TFA 0.30 mL (3.92 mmol) and chloroform (1.2 mL), the contents were heated at 50° C. for 4 h. Concentration of the reaction under vacuum afforded the desired product. ES MS m/z 441 (M+H).

### Example 392

### methyl(2S)-cyclohexyl({[5-phenyl-2-({[(2,4,6-trichlorophenyl)amino]carbonyl}amino)-3-thienyl]carbonyl}amino)ethanoate

To 5-phenyl-2-({[(2,4,6-trichlorophenyl)amino]carbonyl}amino)-3-thiophenecarboxylic acid 0.132 g (0.302 mmol) in DMF (3.0 mL) was added HATU 0.114 g (0.302 mmol) and Hunig's base 0.0527 mL (0.132 mmol), followed by the addition of methyl(2S)-amino(cyclohexyl)ethanoate hydrochloride 0.0627 g (0.302 mmol) and the contents stirred at r.t. for 16 h. The crude reaction mixture was loaded onto an isco column and eluted with a gradient of EtOAc/Hexane (0-60%) over 35 min to afford 0.070 g (40%) of a yellow solid. ES MS m/z 594 (M+H).

### Example 393

### (2S)-cyclohexyl({[5-phenyl-2-({[(2,4,6-trichlorophenyl)amino]carbonyl}amino)-3-thienyl]carbonyl}amino)ethanoic acid

To methyl(2S)-cyclohexyl({[5-phenyl-2-({[(2,4,6-trichlorophenyl)amino]carbonyl}amino)-3-thienyl]carbonyl}amino)ethanoate 0.049 g (0.083 mmol) was added a 1.0 M solution of lithium hydroxide 0.10 mL (0.10 mmol) and the contents stirred for 16 h. The reaction was then acidified to pH=4.0, the precipitated product was then extracted with EtOAc. Drying of the organic layer with magnesium sulfate followed by concentration under vacuum afforded the desired product 0.035 g (73%) as a yellow solid. ES MS m/z 580 (M+H).

### Example 394

### 2-({[(2,6-dichlorophenyl)amino]carbonyl}amino)-5-(1-methylethyl)-3-thiophenecarboxylic acid

### Step 1

### 1,1-dimethylethyl 2-amino-5-(1-methylethyl)-3-thiophenecarboxylate

To a mixture of 3-methylbutanal 2.86 g (0.0333 mole) and 1,1-dimethylethyl cyanoacetate 4.69 g (0.0333 mole) and sulfur 1.17 g (0.0366 mole) in ethanol (50 mL) was added morpholine 3.33 mL (0.038 mole) and the contents heated under nitrogen at 50° C. for 18 h. After filtration, water was added to the reaction to precipitate the desired product. Filtration of the solid followed by washing with 30% aqueous ethanol and drying afforded 2.3 g (28%) of a yellow solid.

### Step 2

### 1,1-dimethylethyl 2-({[(2,6-dichlorophenyl)amino]carbonyl}amino)-5-(1-methylethyl)-3-thiophenecarboxylate

To 1,1-dimethylethyl 2-amino-5-(1-methylethyl)-3-thiophenecarboxylate 0.5 g (2.07 mmole) and 1,3-dichloro-2-isocyanatobenzene 0.429 g (2.27 mmole) was added DMF (3.0 mL) and triethylamine 0.354 mL (2.07 mmole) and the contents heated at 80° C. for 2 h. The crude reaction was loaded onto an isco column and eluted with a gradient of EtOAc/Hexane (0-60%) over 35 min to afford 0.38 g (43%) of a white solid.

### Step 3

### 2-({[(2,6-dichlorophenyl)amino]carbonyl}amino)-5-(1-methylethyl)-3-thiophenecarboxylic acid

To 1,1-dimethylethyl 2-({[(2,6-dichlorophenyl)amino]carbonyl}amino)-5-(l-methylethyl)-3-thiophenecarboxylate 0.150 g (0.350 mmol) was added TFA 0.30 mL (3.90 mmol) and chloroform (1.5 mL), the contents were heated at 50° C. for 4 h. Concentration of the reaction under vacuum afforded the desired product. ES MS m/z 373 (M+H).

### Example 395

### methyl(2S)-cyclohexyl({[2-({[(2,6-dichlorophenyl)amino]carbonyl}amino)-5-(1-methylethyl)-3-thienyl]carbonyl}amino)ethanoate

To 2-({[(2,6-dichlorophenyl)amino]carbonyl}amino)-5-(1-methylethyl)-3-thiophenecarboxylic acid 0.130 g (0.350 mmol) in DMF (3.0 mL) was added HATU 0.133 g (0.350 mmol) and Hunig's base 0.125 mL (0.700 mmol), followed by the addition of methyl(2S)-amino(cyclohexyl)ethanoate hydrochloride 0.073 g (0.350 mmol) and the contents stirred at r.t. for 16 h. The crude reaction mixture was loaded onto an isco column and eluted with a gradient of EtOAc/Hexane (0-100%) over 35 min to afford 0.110 g (60%) of a yellow solid. ES MS m/z 526 (M+H).

### Example 396

### (2S)-cyclohexyl({[2-({[(2,6-dichlorophenyl)amino]carbonyl}amino)-5-(1-methylethyl)-3-thienyl]carbonyl}oxy)ethanoic acid

To methyl(2S)-cyclohexyl({[2-({[(2,6-dichlorophenyl)amino]carbonyl}amino)-5-(1-methylethyl)-3-thienyl]carbonyl}amino)ethanoate 0.095 g (0.181 mmol) was added a 1.0 M solution of lithium hydroxide 0.217 mL (0.217 mmol) and the contents stirred for 16 h. The reaction was then acidified to pH=4.0, the precipitated product was then extracted with EtOAc. Drying of the organic layer with magnesium sulfate followed by concentration under vacuum afforded the desired product 0.060 g (65%) as a yellow solid. ES MS m/z 580 (M+H).

### Example 397

### 2-({[(2,6-dichlorophenyl)amino]carbonyl}amino)-5-(phenylmethyl)-3-thiophenecarboxylic acid

### Step 1

### 1,1-dimethylethyl 2-amino-5-(phenylmethyl)-3-thiophenecarboxylate

To a mixture of 3-phenylpropanal 2.23 g (0.0166 mole) and 1,1-dimethylethyl cyanoacetate 2.34 g (0.0166 mole) and sulfur 0.585 g (0.018 mole) in ethanol (30 mL) was added morpholine 1.66 mL (0.019 mole) and the contents heated under nitrogen at 50° C. for 18 h. After filtration, water followed by EtOAc was added to the reaction mixture. Separation of the organic layer followed by drying with magnesium sulfate and concentration under vacuum gave the crude product which was columned using an isco column eluting with EtOAc/Hexane (0-60%) to afford 2.2 g (46%) a yellow oil.

### Step 2

### 1,1-dimethylethyl 2-({[(2,6-dichlorophenyl)amino]carbonyl}amino)-5-(phenylmethyl)-3-thiophenecarboxylate

To 1,1-dimethylethyl 2-amino-5-(phenylmethyl)-3-thiophenecarboxylate 0.5 g (1.73 mmol) and 1,3-dichloro-2-isocyanatobenzene 0.357 g (1.90 mmole) was added DMF (3.0 mL) and triethylamine 0.266 mL (1.90 mmole) an the contents heated at 80° C. for 2 h. The crude reaction was loaded onto an isco column and eluted with a gradient of EtOAc/Hex (0-60%) over 35 min to afford 0.41 g (50%) of a white solid.

### Step 3

### 2-({[(2,6-dichlorophenyl)amino]carbonyl}amino)-5-(phenylmethyl)-3-thiophenecarboxylic acid

To 1,1-dimethylethyl 2-({[(2,6-dichlorophenyl)amino]carbonyl}amino)-5-(phenylmethyl)-3-thiophenecarboxylate 0.150 g (0.315 mmol) was added TFA 0.30 mL (3.90 mmol) and chloroform (1.5 mL), the contents were heated at 50° C. for 3 h. Concentration of the reaction under vacuum afforded the desired product. ES MS m/z 421 (M+H).

### Example 398

### methyl(2S)-cyclohexyl({[2-({[(2,6-dichlorophenyl)amino]carbonyl}amino)-5-(phenylmethyl)-3-thienyl]carbonyl}amino)ethanoate

To 2-({[(2,6-dichlorophenyl)amino]carbonyl}amino)-5-(phenylmethyl)-3-thiophenecarboxylic acid 0.132 g (0.315 mmol) in DMF (3.0 mL) was added HATU 0.119 g (0.315 mmol) and Hunig's base 0.112 mL (0.630 mmol), followed by the addition of methyl(2S)-amino(cyclohexyl)ethanoate hydrochloride 0.065 g (0.315 mmol) and the contents stirred at r.t. for 16 h. The crude reaction mixture was loaded onto an isco column and eluted with a gradient of EtOAc/Hex (0-60%) over 35 min to afford 0.113 g (63%) of a white solid. ES MS m/z 575 (M+H).

### Example 399

### (2S)-cyclohexyl({[2-({[(2,6-dichlorophenyl)amino]carbonyl}amino)-5-(phenylmethyl)-3-thienyl]carbonyl}oxy)ethanoic acid

To methyl(2S)-cyclohexyl({[2-({[(2,6-dichlorophenyl)amino]carbonyl}amino)-5-(phenylmethyl)-3-thienyl]carbonyl}amino)ethanoate 0.075 g (0.130 mmol) was added a 1.0 M solution of lithium hydroxide 0.169 mL (0.169 mmol) and the contents stirred for 16 h. The reaction was then acidified to pH=4.0, the precipitated product was then extracted with EtOAc. Drying of the organic layer with magnesium sulfate followed by concentration under vacuum afforded to desired product 0.050 g (68%) as a yellow solid. ES MS m/z 561 (M+H).

### Example 400

### 3-({[(2,6-dichlorophenyl)amino]carbonyl}amino)-5-(4-pyridinyl)-2-thiophenecarboxylic acid

### Step 1

### (2E)-3-chloro-3-(4-pyridinyl)-2-propenenitrile

To DMF (16.4 mL) cooled to 0° C. was added phosphorousoxychloride 9.89 mL (0.106 mole) and the contents stirred for 10 mins. To the cooled reaction was added 1-(4-pyridinyl)ethanone 4.0 g (0.0244 mole). After warming to r.t., the reaction mixture was heated at 500° C. for 10 mins. The reaction was then cooled to 0° C. and hydroxylamine hydrochloride 11.78 g (0.169 mole) was added slowly. After stirring for 5 mins., the contents were heated at 120° C. for 15 mins. On cooling to r.t., EtOAc was added followed by neutralization with satd. NaHCO₃. The organic layer was separated and the aqueous layer was extracted with EtOAc. The organic layer was dried, and then concentrated under vacuum to afford a brown oil that was carried on to the next step.

### Step 2

### methyl 3-amino-5-(4-pyridinyl)-2-thiophenecarboxylate

To methanol (60 mL) was added a 25% solution of sodium methoxide in methanol 6.85 mL (0.0317 mole) and methyl mercaptoacetate 2.19 mL (0.0244 mole) under nitrogen followed by the addition of (2E)-3-chloro-3-(4-pyridinyl)-2-propenenitrile 4.0 g (0.0244 mole) in DMF (20 mL) at 0° C. After stirring for 30 mins., water was added to precipitate the desired product. After filtration and washing with water, the product was dried under vacuum 2.1 g (37%).

### Step 3

### 3-amino-5-(4-pyridinyl)-2-thiophenecarboxylic acid

To methyl 3-amino-5-(4-pyridinyl)-2-thiophenecarboxylate 1.0 g (4.27 mmol) was added 1.0 M solution of lithium hydroxide 5.46 mL (5.55 mmol) and dioxane 10 mL. After refluxing for 2 h, the reaction was cooled and then acidified with 1N HCl. The precipitated product was filtered, dried and carried on to the next step.

### Step 4

### 3-({[(2,6-dichlorophenyl)amino]carbonyl}amino)-5-(4-pyridinyl)-2-thiophenecarboxylic acid

To a solution of 3-amino-5-(4-pyridinyl)-2-thiophenecarboxylic acid 0.3 g (1.36 mmol) in DMF (2.0 mL) was added 1,3-dichloro-2-isocyanatobenzene 0.282 g (1.49 mmole) and triethylamine 0.209 mL (1.36 mmol). After heating at 80° C. for 2 h, the reaction mixture was acidified with 1N HCl to pH=6 and then filtered. The filtered solid was washed with water, ether and EtOAc and then dried under vacuum to afford 0.513 g (100%) of the desired product as a white solid. ES MS m/z 408 (M+H).

### Example 401

### methyl(2S)-cyclohexyl({[3-({[(2,6-dichlorophenyl)amino]carbonyl}amino)-5-(4-pyridinyl)-2-thienyl]carbonyl}amino)ethanoate

To 3-({[(2,6-dichlorophenyl)amino]carbonyl}amino)-5-(4-pyridinyl)-2-thiophenecarboxylic acid 0.150 g (0.368 mmol) in DMF (3.0 mL) was added HATU 0.153 g (0.404 mmol) and Hunig's base 0.079 mL (0.441 mmol), followed by the addition of methyl(2S)-amino(cyclohexyl)ethanoate hydrochloride 0.077 g (0.368 mmol) and the contents stirred at r.t. for 16 h. The crude reaction mixture was loaded onto an isco column and eluted with a gradient of EtOAc/Hex (0-100%) over 35 min to afford 0.120 g (58%) of a white solid. ES MS m/z 562 (M+H).

### Example 402

### (2S)-cyclohexyl({[3-({[(2,6-dichlorophenyl)amino]carbonyl}amino)-5-(4-pyridinyl)-2-thienyl]carbonyl}oxy)ethanoic acid

To methyl(2S)-cyclohexyl({[3-({[(2,6-dichlorophenyl)amino]carbonyl}amino)-5-(4-pyridinyl)-2-thienyl]carbonyl}amino)ethanoate 0.075 g (0.133 mmol) was added a 1.0 M solution of lithium hydroxide 0.173 mL (0.173 mmol) and the contents stirred for 16 h. The reaction was then acidified to pH=4.0, the precipitated product was then extracted with EtOAc. Drying of the organic layer with magnesium sulfate followed by concentration under vacuum afforded to desired product 0.051 g (70%) as a yellow solid. ES MS m/z 548 (M+H).

### Example 403

### 3-({[(2,6-dichlorophenyl)amino]carbonyl}amino)-5-(3-pyridinyl)-2-thiophenecarboxylic acid

### Step 1

### (2E)-3-chloro-3-(3-pyridinyl)-2-propenenitrile

To DMF (16.4 mL) cooled to 0° C. was added phosphorousoxychloride 9.89 mL (0.106 mole) and the contents stirred for 10 mins. To the cooled reaction was added 1-(3-pyridinyl)ethanone 4.0 g (0.0244 mole). After warming to r.t., the reaction mixture was heated at 50° C. for 10 mins. The reaction was then cooled to 0° C. and hydroxylamine hydrochloride 11.78 g (0.169 mole) was added slowly. After stirring for 5 mins., the contents were heated at 120° C. for 15 mins. On cooling to r.t., EtOAc was added followed by neutralization with satd. NaHCO₃. The organic layer was separated and the aqueous layer was extracted with EtOAc. The organic layer was dried, and then concentrated under vacuum to afford a brown oil that was carried on to the next step.

### Step 2

### methyl 3-amino-5-(3-pyridinyl)-2-thiophenecarboxylate

To methanol (50 mL) was added a 25% solution of sodium methoxide in methanol 6.40 mL (0.0296 mole) and methyl mercaptoacetate 2.04 mL (0.0228 mole) under nitrogen followed by the addition of (2E)-3-chloro-3-(3-pyridinyl)-2-propenenitrile 3.74 g (0.0228 mole) in DMF (20 mL) at 0° C. After stirring for 30 mins., water was added to precipitate the desired product. After filtration and washing with water, the product was dried under vacuum 2.8 g (53%).

### Step 3

### 3-amino-5-(3-pyridinyl)-2-thiophenecarboxylic acid

To methyl 3-amino-5-(3-pyridinyl)-2-thiophenecarboxylate 1.0 g (4.27 mmol) was added 1.0 M solution of lithium hydroxide 5.46 mL (5.55 mmol) and dioxane 10 mL. After refluxing for 2 h, the reaction was cooled and then acidified with 1N HCl. The precipitated product was filtered, dried and carried on to the next step.

### Step 4

### 3-({[(2,6-dichlorophenyl)amino]carbonyl}amino)-5-(3-pyridinyl)-2-thiophenecarboxylic acid

To a solution of 3-amino-5-(3-pyridinyl)-2-thiophenecarboxylic acid 0.3 g (1.36 mmol) in DMF (2.0 mL) was added 1,3-dichloro-2-isocyanatobenzene 0.282 g (1.49 mmole) and triethylamine 0.209 mL (1.36 mmol). After heating at 800° C. for 2 h, the reaction mixture was acidified with 1N HCl to pH=6 and then filtered. The filtered solid was washed with water, ether and EtOAc and then dried under vacuum to afford 0.563 g of the desired product as a yellow solid. ES MS m/z 408 (M+H).

### Example 404

### methyl(2S)-cyclohexyl({[3-({[(2,6-dichlorophenyl)amino]carbonyl}amino)-5-(3-pyridinyl)-2-thienyl]carbonyl}amino)ethanoate

To 3-({[(2,6-dichlorophenyl)amino]carbonyl}amino)-5-(3-pyridinyl)-2-thiophenecarboxylic acid 0.150 g (0.368 mmol) in DMF (3.0 mL) was added HATU 0.153 g (0.404 mmol) and Hunig's base 0.079 mL (0.441 mmol), followed by the addition of methyl(2S)-amino(cyclohexyl)ethanoate hydrochloride 0.077 g (0.368 mmol) and the contents stirred at r.t. for 16 h. The crude reaction mixture was loaded onto an isco column and eluted with a gradient of EtOAc/Hexane (0-100%) over 35 min to afford 0.090 g (44%) of a white solid. ES MS m/z 562 (M+H).

### Example 405

### (2S)-cyclohexyl({[3-({[(2,6-dichlorophenyl)amino]carbonyl}amino)-5-(3-pyridinyl)-2-thienyl]carbonyl}oxy)ethanoic acid

To methyl(2S)-cyclohexyl({[3-({[(2,6-dichlorophenyl)amino]carbonyl}amino)-5-(3-pyridinyl)-2-thienyl]carbonyl}amino)ethanoate 0.075 g (0.133 mmol) was added a 1.0 M solution of lithium hydroxide 0.173 mL (0.173 mmol) and the contents stirred for 16 h. The reaction was then acidified to pH=4.0, the precipitated product was then extracted with EtOAc. Drying of the organic layer with magnesium sulfate followed by concentration under vacuum afforded to desired product 0.046 g (63%) as a yellow solid. ES MS m/z 548 (M+H).

### Example 406

### 5-(4-cyanophenyl)-3-({[(2,6-dichlorophenyl)amino]carbonyl}amino)-2-thiophenecarboxylic acid

### Step 1

### 4-[(E)-1-chloro-2-cyanoethenyl]benzonitrile

To DMF (16.4 mL) cooled to 0° C. was added phosphorousoxychloride 9.89 mL (0.106 mole) and the contents stirred for 10 mins. To the cooled reaction was added 4-acetylbenzonitrile 6.15 g (0.0424 mole). After warming to r.t., the reaction mixture was heated at 500° C. for 10 mins. The reaction was then cooled to 0° C. and hydroxylamine hydrochloride 11.78 g (0.169 mole) was added slowly. After stirring for 5 mins., the contents were heated at 120° C. for 15 mins. On cooling to r.t., EtOAc was added followed by neutralization with satd. NaHCO₃. The organic layer was separated and the aqueous layer was extracted with EtOAc. The organic layer was dried, and then concentrated under vacuum to afford a brown oil that was carried on to the next step.

### Step 2

### methyl 3-amino-5-(4-cyanophenyl)-2-thiophenecarboxylate

To methanol (60 mL) was added a 25% solution of sodium methoxide in methanol 4.77 mL (0.0222 mole) and methyl mercaptoacetate 1.53 mL (0.0187 mole) under nitrogen followed by the addition of 4-[(E)-1-chloro-2-cyanoethenyl]benzonitrile 3.20 g (0.017 mole) in DMF (20 mL) at 0° C. After stirring for 30 mins., water was added to precipitate the desired product. After filtration and washing with water, the product was dried under vacuum 2.6 g (59%).

### Step 3

### 3-amino-5-(4-cyanophenyl)-2-thiophenecarboxylic acid

To methyl 3-amino-5-(4-cyanophenyl)-2-thiophenecarboxylate 1.5 g (5.81 mmol) was added 1.0 M solution of lithium hydroxide 6.39 mL (6.39 mmol) and dioxane 10 mL. After refluxing for 2 h, the reaction was cooled and then acidified with 1N HCl. The precipitated product was filtered, dried and carried on to the next step.

### Step 4

### 5-(4-cyanophenyl)-3-({[(2,6-dichlorophenyl)amino]carbonyl}amino)-2-thiophenecarboxylic acid

To a solution of 3-amino-5-(4-cyanophenyl)-2-thiophenecarboxylic acid 0.5 g (2.04 mmol) in DMF (3.0 mL) was added 1,3-dichloro-2-isocyanatobenzene 0.385 g (2.04 mmole) and triethylamine 0.314 mL (2.24 mmol). After heating at 80° C. for 2 h, the reaction mixture was acidified with 1N HCl to pH=6 and then filtered. The filtered solid was washed with water, ether and EtOAC and then dried under vacuum to afford 0.521 g of the desired product as a yellow solid. ES MS m/z 432 (M+H).

### Example 407

### (2S)-({[5-(4-cyanophenyl)-3-({[(2,6-dichlorophenyl)amino]carbonyl}amino)-2-thienyl]carbonyl}amino)(cyclohexyl)ethanoic acid

### Step 1

### methyl(2S)-({[5-(4-cyanophenyl)-3-({[(2,6-dichlorophenyl)amino]carbonyl}amino)-2-thienyl]carbonyl}amino)(cyclohexyl)ethanoate

To 5-(4-cyanophenyl)-3-({[(2,6-dichlorophenyl)amino]carbonyl}amino)-2-thiophenecarboxylic acid 0.195 g (0.452 mmol) in DMF (3.0 mL) was added HATU 0.189 g (0.497 mmol) and Hunig's base 0.241 mL (1.35 mmol), followed by the addition of methyl(2S)-amino(cyclohexyl)ethanoate hydrochloride 0.094 g (0.452 mmol) and the contents stirred at r.t. for 16 h. The crude reaction mixture was loaded onto an isco column and eluted with a gradient of EtOAc/Hexane (0-100%) over 35 min to afford 0.171 g (65%) of a yellow solid.

### Step 2

### (2S)-({[5-(4-cyanophenyl)-3-({[(2,6-dichlorophenyl)amino]carbonyl}amino)-2-thienyl]carbonyl}amino)(cyclohexyl)ethanoic acid

To methyl(2S)-({[5-(4-cyanophenyl)-3-({[(2,6-dichlorophenyl)amino]carbonyl}amino)-2-thienyl]carbonyl}amino)(cyclohexyl)ethanoate 0.020 g (0.0342 mmol) was added a 1.0 M solution of lithium hydroxide 0.064 mL (0.064 mmol) and the contents stirred for 16 h. The reaction was then acidified to pH=4.0, the precipitated product was then extracted with EtOAc. Drying of the organic layer with magnesium sulfate followed by concentration under vacuum afforded to desired product 0.012 g (61%) as a yellow solid. ES MS m/z 571 (M+H).

### Example 408

### 3-({[(2,6-dichlorophenyl)amino]carbonyl}amino)-5-[4-(methyloxy)phenyl]-2-thiophenecarboxylic acid

### Step 1

### (2E)-3-chloro-3-[4-(methyloxy)phenyl]-2-propenenitrile

To DMF (16.4 mL) cooled to 0° C. was added phosphorousoxychloride 9.89 mL (0.106 mole) and the contents stirred for 10 mins. To the cooled reaction was added 1-[4-(methyloxy)phenyl]ethanone 6.36 g (0.0424 mole). After warming to r.t., the reaction mixture was heated at 50° C. for 10 mins. The reaction was then cooled to 0° C. and hydroxylamine hydrochloride 11.78 g (0.169 mole) was added slowly. After stirring for 5 mins., the contents were heated at 120° C. for 15 mins. On cooling to r.t., EtOAc was added followed by neutralization with satd. NaHCO₃. The organic layer was separated and the aqueous layer was extracted with EtOAc. The organic layer was dried, and then concentrated under vacuum to afford a brown oil that was carried on to the next step.

### Step 2

### methyl 3-amino-5-[4-(methyloxy)phenyl]-2-thiophenecarboxylate

To methanol (60 mL) was added a 25% solution of sodium methoxide in methanol 9.42 mL (0.0436 mole) and methyl mercaptoacetate 3.02 mL (0.0336 mole) under nitrogen followed by the addition of (2E)-3-chloro-3-[4-(methyloxy)phenyl]-2-propenenitrile 6.5 g (0.0336 mole) in DMF (30 mL) at 0° C. After stirring for 30 mins., water was added to precipitate the desired product. After filtration and washing with water, the product was dried under vacuum 4.8 g (55%).

### Step 3

### 3-amino-5-[4-(methyloxy)phenyl]-2-thiophenecarboxylic acid

To methyl 3-amino-5-[4-(methyloxy)phenyl]-2-thiophenecarboxylate 1.5 g (5.70 mmol) was added 1.0 M solution of lithium hydroxide 6.84 mL (6.84 mmol) and dioxane 10 mL. After refluxing for 2 h, the reaction was cooled and then acidified with 1N HCl. The precipitated product was filtered, dried and carried on to the next step.

### Step 4

### 3-({[(2,6-dichlorophenyl)amino]carbonyl}amino)-5-[4-(methyloxy)phenyl]-2-thiophenecarboxylic acid

To a solution of 3-amino-5-[4-(methyloxy)phenyl]-2-thiophenecarboxylic acid 0.5 g (2.0 mmol) in DMF (3.0 mL) was added 1,3-dichloro-2-isocyanatobenzene 0.377 g (2.0 mmole) and triethylamine 0.280 mL (2.0 mmol). After heating at 800° C. for 2 h, the reaction mixture was acidified with 1N HCl to pH=6 and then filtered. The filtered solid was washed with water, ether and EtOAc and then dried under vacuum to afford 0.521 g of the desired product as a yellow solid. ES MS m/z 437 (M+H).

### Example 409

### (2S)-cyclohexyl[({3-({[(2,6-dichlorophenyl)amino]carbonyl}amino)-5-[4-(methyloxy)phenyl]-2-thienyl}carbonyl)amino]ethanoic acid

### Step 1

### methyl(2S)-cyclohexyl[({3-({[(2,6-dichlorophenyl)amino]carbonyl}amino)-5-[4-(methyloxy)phenyl]-2-thienyl}carbonyl)amino]ethanoate

To 3-({[(2,6-dichlorophenyl)amino]carbonyl}amino)-5-[4-(methyloxy)phenyl]-2-thiophenecarboxylic acid 0.167 g (0.383 mmol) in DMF (3.0 mL) was added HATU 0.146 g (0.383 mmol) and Hunig's base 0.068 mL (0.383 mmol), followed by the addition of methyl(2S)-amino(cyclohexyl)ethanoate hydrochloride 0.080 g (0.383 mmol) and the contents stirred at r.t. for 16 h. The crude reaction mixture was loaded onto an isco column and eluted with a gradient of EtOAc/Hexane (0-100%) over 35 min to afford 0.120 g (53%) of a yellow solid.

### Step 2

### (2S)-cyclohexyl[({3-({[(2,6-dichlorophenyl)amino]carbonyl}amino)-5-[4-(methyloxy)phenyl]-2-thienyl}carbonyl)amino]ethanoic acid

To methyl(2S)-cyclohexyl[({3-({[(2,6-dichlorophenyl)amino]carbonyl}amino)-5-[4-(methyloxy)phenyl]-2-thienyl}carbonyl)amino]ethanoate 0.10 g (0.170 mmol) was added a 1.0 M solution of lithium hydroxide 0.338 mL (0.338 mmol) and the contents stirred for 16 h. The reaction was then acidified to pH=4.0, the precipitated product was then extracted with EtOAc. Drying of the organic layer with magnesium sulfate followed by concentration under vacuum afforded to desired product 0.012 g (61%) as a yellow solid. ES MS m/z 576 (M+H).

### Example 410

### 3-({[(2,6-dichlorophenyl)amino]carbonyl}amino)-5-phenyl-2-thiophenecarboxylic acid

To a solution of 3-amino-5-phenyl-2-thiophenecarboxylic acid 0.5 g (2.28 mmol) in DMF (3 mL) was added 1,3-dichloro-2-isocyanatobenzene 0.428 g (2.28 mmol) and triethylamine 0.319 mL (2.28 mmol) and the contents heated to 80° C. for 2 h. After addition of sat. Na₂CO₃ (20 mL), water and EtOAc were added and the layers separated. The aqueous solution was acidified and then extracted with EtOAc and dried over magnesium sulfate followed by concentration under vacuum to afford the desired product as an off white solid 0.64 g (71%). ES MS m/z 407 (M+H).

### Example 411

### (2S)-cyclohexyl({[3-({[(2,6-dichlorophenyl)amino]carbonyl}amino)-5-phenyl-2-thienyl]carbonyl}amino)ethanoic acid

### Step 1

### methyl(2S)-cyclohexyl({[3-({[(2,6-dichlorophenyl)amino]carbonyl}amino)-5-phenyl-2-thienyl]carbonyl}amino)ethanoate

To 3-({[(2,6-dichlorophenyl)amino]carbonyl}amino)-5-phenyl-2-thiophenecarboxylic acid 0.20 g (0.492 mmol) in DMF (3.0 mL) was added HATU 0.187 g (0.492 mmol) and Hunig's base 0.171 mL (0.984 mmol), followed by the addition of methyl(2S)-amino(cyclohexyl)ethanoate hydrochloride 0.102 g (0.492 mmol) and the contents stirred at r.t. for 16 h. The crude reaction mixture was loaded onto an isco column and eluted with a gradient of EtOAc/Hexane (0-100%) over 35 min to afford 0.145 g (53%) of a yellow solid.

### Step 2

### (2S)-cyclohexyl({[3-({[(2,6-dichlorophenyl)amino]carbonyl}amino)-5-phenyl-2-thienyl]carbonyl}amino)ethanoic acid

To methyl(2S)-cyclohexyl({[3-({[(2,6-dichlorophenyl)amino]carbonyl}amino)-5-phenyl-2-thienyl]carbonyl}amino)ethanoate 0.10 g (0.178 mmol) was added a 1.0 M solution of lithium hydroxide 0.534 mL (0.534 mmol) and the contents stirred for 16 h. The reaction was then acidified to pH=4.0, the precipitated product was then extracted with EtOAc. Drying of the organic layer with magnesium sulfate followed by concentration under vacuum afforded the desired product 0.080 g (82%) as a yellow solid. ES MS m/z 546 (M+H).

### Example 412

### 2-({[(2,6-dichlorophenyl)amino]carbonyl}amino)-5-[4-(methyloxy)phenyl]-3-thiophenecarboxylic acid

### Step 1

### 1,1-dimethylethyl 2-amino-3-thiophenecarboxylate

To 1,1-dimethylethyl cyanoacetate 19.8 g (140 mmol) and methyl mercaptoacetate 10.66 g (70 mmol) in DMF (100 mL) was added triethylamine 19.72 mL (140 mmol) dropwise over 15 mins and the contents heated at 450° C. for 45 mins. After the addition of water, EtOAc was added. Washing of the organic layer with water followed by drying with magnesium sulfate and concentration under vacuum afforded the crude product. The crude product was purified using an isco column eluting with EtOAc/Hexane (0-60%) to afford 21 g (75%) of a white solid.

### Step 2

### 1,1-dimethylethyl 2-[(trifluoroacetyl)amino]-3-thiophenecarboxylate

To 1,1-dimethylethyl 2-amino-3-thiophenecarboxylate 9.86 g (0.049 mole) in DCM (100 mL) was added Hunig's base 11.21 mL (0.064 mole). After cooling the contents to 0° C., TFAA 7.73 mL (0.054 mmol) was added dropwise. After stirring for 2 h, the reaction mixture was washed with water, dried with magnesium sulfate and then concentrated under vacuum to afford the desired product in quantative yield.

### Step 3

### 1,1-dimethylethyl 5-bromo-2-[(trifluoroacetyl)amino]-3-thiophenecarboxylate

To 1,1-dimethylethyl 2-[(trifluoroacetyl)amino]-3-thiophenecarboxylate 4.0 g (0.005 mole) in dioxane (80 mL) cooled to 0° C. was added, bromine 0.68 mL (0.005 mole) dropwise over 15 mins. After stirring for 15 mins, EtOAc was added (200 mL) followed by the addition of water. The organic layer was washed with brine and then dried with magnesium sulfate. Concentration of the organic layer under vacuum afforded 4.2 g (84%) of the bromide.

### Step 4

### 1,1-dimethylethyl 2-amino-5-bromo-3-thiophenecarboxylate

To 1,1-dimethylethyl 5-bromo-2-[(trifluoroacetyl)amino]-3-thiophenecarboxylate 4.2 g (0.01126 mole) was added MeOH (40 mL) and water (20 mL). To the reaction mixture was then added K₂CO₃ 4.67 g (0.0337 mole) and the contents stirred under nitrogen for 8 h. After adding EtOAc, the organic layer was separated, dried with magnesium sulfate and concentrated to afford the crude product that was carried to the next step.

### Step 5

### 1,1-dimethylethyl 5-bromo-2-({[(2,6-dichlorophenyl)amino]carbonyl}amino)-3-thiophenecarboxylate

To a solution of 1,1-dimethylethyl 2-amino-5-bromo-3-thiophenecarboxylate 2.21 g (7.98 mmole) in DMF (15 mL) was added 1,3-dichloro-2-isocyanatobenzene 1.5 g (7.98 mole) and triethylamine 1.23 mL (8.77 mmol) and the contents heated to 80° C. for 2 h. The crude mixture was loaded onto an isco column and eluted with EtOAc/Hexane (0-60%) to afford 2.1 g (57%) of a white solid.

### Step 6

### 1,1-dimethylethyl 2-({[(2,6-dichlorophenyl)amino]carbonyl}amino)-5-[4-(methyloxy)phenyl]-3-thiophenecarboxylate

To a solution of 1,1-dimethylethyl 5-bromo-2-({[(2,6-dichlorophenyl)amino]carbonyl}amino)-3-thiophenecarboxylate 0.3 g (0.645 mmol) in DME (5 mL) was added [4-(methyloxy)phenyl]boronic acid 0.147 g (0.967 mmol) followed by a solution of 2M Na₂CO₃ 1.29 mL and Dichlorobis(triphenylphosphine)palladium(II) 0.05 g (10 mole %) and the contents refluxed under nitrogen for 6 h. Filtration of the reaction mixture through celite followed by washing with EtOAc and concentration afforded the crude product which was loaded onto an isco column and eluted with EtOAc/Hexane (0-80%) to afford 0.210 g (66%) of a white solid.

### Step 7

### 2-({[(2,6-dichlorophenyl)amino]carbonyl}amino)-5-[4-(methyloxy)phenyl]-3-thiophenecarboxylic acid

To 1,1-dimethylethyl 2-({[(2,6-dichlorophenyl)amino]carbonyl}amino)-5-[4-(methyloxy)phenyl]-3-thiophenecarboxylate 0.145 g (0.294 mmol) was added TFA 0.3 mL (3.90 mmol) and chloroform (1.0 mL) and the contents heated at 500° C. for 2 h. Concentration of the reaction under vacuum afforded the desired product. ES MS m/z 437 (M+H).

### Example 413

### 5-bromo-2-({[(2,6-dichlorophenyl)amino]carbonyl}amino)-3-thiophenecarboxylic acid

To a solution of 1,1-dimethylethyl 5-bromo-2-({[(2,6-dichlorophenyl)amino]carbonyl}amino)-3-thiophenecarboxylate 0.035 g (0.075 mmol) was added TFA 0.3 mL (3.89 mmol) and the contents heated at 60° C. for 2 h. Concentration of the reaction under vacuum gave the desired product. ES MS m/z 410 (M+H).

### Example 414

### 2-({[(2,6-dichlorophenyl)amino]carbonyl}amino)-5-(3-pyridinyl)-3-thiophenecarboxylic acid

### Step 1

### 1,1-dimethylethyl 2-({[(2,6-dichlorophenyl)amino]carbonyl}amino)-5-(3-pyridinyl)-3-thiophenecarboxylate

To a solution of 1,1-dimethylethyl 5-bromo-2-({[(2,6-dichlorophenyl)amino]carbonyl}amino)-3-thiophenecarboxylate 0.3 g (0.645 mmol) in DME (5 mL) was added 3-pyridinylboronic acid 0.118 g (0.967 mmol) followed by a solution of 2M Na₂CO₃ 1.29 mL and Dichlorobis(triphenylphosphine)palladium(II) 0.05 g (10 mole %) and the contents refluxed under nitrogen for 6 h. Filtration of the reaction mixture through celite followed by washing with EtOAc and concentration afforded the crude product which was loaded onto an isco column and eluted with EtOAc/Hexane (0-80%) to afford 0.210 g (43%) of a white solid.

### Step 2

### 2-({[(2,6-dichlorophenyl)amino]carbonyl}amino)-5-(3-pyridinyl)-3-thiophenecarboxylic acid

To 1,1-dimethylethyl 2-({[(2,6-dichlorophenyl)amino]carbonyl}amino)-5-[3-(methyloxy)phenyl]-3-thiophenecarboxylate 0.10 g (0.215 mmol) was added TFA 0.3 mL (3.98 mmol) and chloroform (1.0 mL) and the contents heated at 50° C. for 2 h. Concentration of the reaction under vacuum afforded the desired product. ES MS m/z 408 (M+H).

### Example 415

### (2S)-cyclohexyl({[2-({[(2,6-dichlorophenyl)amino]carbonyl}amino)-5-(3-pyridinyl)-3-thienyl]carbonyl}amino)ethanoic acid

### Step 1

### methyl(2S)-cyclohexyl({[2-({[(2,6-dichlorophenyl)amino]carbonyl}amino)-5-(3-pyridinyl)-3-thienyl]carbonyl}amino)ethanoate

To 2-({[(2,6-dichlorophenyl)amino]carbonyl}amino)-5-(3-pyridinyl)-3-thiophenecarboxylic acid 0.080 g (0.196 mmol) in DMF (2.0 mL) was added HATU 0.082 g (0.215 mmol) and Hunig's base 0.101 mL (0.588 mmol), followed by the addition of methyl(2S)-amino(cyclohexyl)ethanoate hydrochloride 0.041 g (0.196 mmol) and the contents stirred at r.t. for 16 h. The crude reaction mixture was loaded onto an isco column and eluted with a gradient of EtOAc/Hexane (0-100%) over 35 min to afford 0.045 g (41%) of a yellow solid.

### Step 2

### (2S)-cyclohexyl({[2-({[(2,6-dichlorophenyl)amino]carbonyl}amino)-5-(3-pyridinyl)-3-thienyl]carbonyl}amino)ethanoic acid

To methyl(2S)-cyclohexyl({[2-({[(2,6-dichlorophenyl)amino]carbonyl}amino)-5-(3-pyridinyl)-3-thienyl]carbonyl}amino)ethanoate 0.100 g (0. mmol) was added a 1.0 M solution of lithium hydroxide 1.0 mL (1.06 mmol) and THF (1.0 mL) and the contents stirred for 16 h. The reaction was then acidified to pH=4.0, the precipitated product was then extracted with EtOAc. Drying of the organic layer with magnesium sulfate followed by concentration under vacuum afforded to desired product 0.096 g (99%) as a yellow solid (U22007-80-2). ES MS m/z 546 (M+H).

### Example 416

### 2-({[(2,6-dichlorophenyl)amino]carbonyl}amino)-5-[3-(methyloxy)phenyl]-3-thiophenecarboxylic acid

### Step 1

### 1,1-dimethylethyl 2-({[(2,6-dichlorophenyl)amino]carbonyl}amino)-5-[3-(methyloxy)phenyl]-3-thiophenecarboxylate

To a solution of 1,1-dimethylethyl 5-bromo-2-({[(2,6-dichlorophenyl)amino]carbonyl}amino)-3-thiophenecarboxylate 0.4 g (0.860 mmol) in DME (7 mL) was added [3-(methyloxy)phenyl]boronic acid 0.196 g (1.29 mmol) followed by a solution of 2M Na₂CO₃ 1.72 mL and Dichlorobis(triphenylphosphine)palladium(II) 0.06 g (10 mole %) and the contents refluxed under nitrogen for 6 h. Filtration of the reaction mixture through celite followed by washing with EtOAc and concentration afforded the crude product which was loaded onto an isco column and eluted with EtOAc/Hexane (0-80%) to afford 0.210 g (50%) of a white solid.

### Step 2

### 2-({[(2,6-dichlorophenyl)amino]carbonyl}amino)-5-[3-(methyloxy)phenyl]-3-thiophenecarboxylic acid

To 1,1-dimethylethyl 2-({[(2,6-dichlorophenyl)amino]carbonyl}amino)-5-[4-(methyloxy)phenyl]-3-thiophenecarboxylate 0.180 g (0.365 mmol) was added TFA 0.5 mL (6.51 mmol) and chloroform (2.0 mL) and the contents heated at 500° C. for 2 h. Concentration of the reaction under vacuum afforded the desired product in quantative yield. ES MS m/z 437 (M+H).

### Example 417

### (2S)-cyclohexyl[({2-({[(2,6-dichlorophenyl)amino]carbonyl}amino)-5-[3-(methyloxy)phenyl]-3-thienyl}carbonyl)amino]ethanoic acid

### Step 1

### methyl(2S)-cyclohexyl[({2-({[(2,6-dichlorophenyl)amino]carbonyl}amino)-5-[3-(methyloxy)phenyl]-3-thienyl}carbonyl)amino]ethanoate

To 2-({[(2,6-dichlorophenyl)amino]carbonyl}amino)-5-[3-(methyloxy)phenyl]-3-thiophenecarboxylic acid 0.159 g (0.365 mmol) in DMF (3.0 mL) was added HATU 0.138 g (0.365 mmol) and Hunig's base 0.254 mL (1.46 mmol), followed by the addition of methyl(2S)-amino(cyclohexyl)ethanoate hydrochloride 0.076 g (0.365 mmol) and the contents stirred at r.t. for 16 h. The crude reaction mixture was loaded onto an isco column and eluted with a gradient of EtOAc/Hex (0-60%) over 35 min to afford 0.161 g (75%) of a yellow solid.

### Step 2

### (2S)-cyclohexyl[({2-({[(2,6-dichlorophenyl)amino]carbonyl}amino)-5-[3-(methyloxy)phenyl]-3-thienyl}carbonyl)amino]ethanoic acid

To methyl(2S)-cyclohexyl[({2-({[(2,6-dichlorophenyl)amino]carbonyl}amino)-5-[3-(methyloxy)phenyl]-3-thienyl}carbonyl)amino]ethanoate 0.100 g (0. mmol) was added a 1.0 M solution of lithium hydroxide 1.96 mL (1.96 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded to desired product 0.10 g (64%) as a yellow solid. ES MS m/z 576 (M+H)

### Example 418

### 2-({[(2,6-dichlorophenyl)amino]carbonyl}amino)-5-(4-fluorophenyl)-3-thiophenecarboxylic acid

### Step 1

### 1,1-dimethylethyl 2-({[(2,6-dichlorophenyl)amino]carbonyl}amino)-5-(4-fluorophenyl)-3-thiophenecarboxylate

To a solution of 1,1-dimethylethyl 5-bromo-2-({[(2,6-dichlorophenyl)amino]carbonyl}amino)-3-thiophenecarboxylate 0.4 g (0.860 mmol) in DME (7 mL) was added (4-fluorophenyl)boronic acid 0.180 g (1.29 mmol) followed by a solution of 2M Na₂CO₃ 1.72 mL and Dichlorobis(triphenylphosphine)palladium(II) 0.06 g (10 mole %) and the contents refluxed under nitrogen for 6 h. Filtration of the reaction mixture through celite followed by washing with EtOAc and concentration afforded the crude product which was loaded onto an isco column and eluted with EtOAc/Hexane (0-80%) to afford 0.230 g (56%) of a white solid.

### Step 2

### 2-({[(2,6-dichlorophenyl)amino]carbonyl}amino)-5-(4-fluorophenyl)-3-thiophenecarboxylic acid

To 1,1-dimethylethyl 2-({[(2,6-dichlorophenyl)amino]carbonyl}amino)-5-(4-fluorophenyl)-3-thiophenecarboxylate 0.207 g (0.431 mmol) was added TFA 0.3 mL (3.91 mmol) and chloroform (2.0 mL) and the contents heated at 500° C. for 2 h. Concentration of the reaction under vacuum afforded the desired product in quantative yield. ES MS m/z 425 (M+H).

### Example 419

### (2S)-cyclohexyl({[2-({[(2,6-dichlorophenyl)amino]carbonyl}amino)-5-(4-fluorophenyl)-3-thienyl]carbonyl}amino)ethanoic acid

### Step 1

### methyl(2S)-cyclohexyl({[2-({[(2,6-dichlorophenyl)amino]carbonyl}amino)-5-(4-fluorophenyl)-3-thienyl]carbonyl}amino)ethanoate

To 2-({[(2,6-dichlorophenyl)amino]carbonyl}amino)-5-(4-fluorophenyl)-3-thiophenecarboxylic acid 0.206 g (0.488 mmol) in DMF (3.0 mL) was added HATU 0.185 g (0.488 mmol) and Hunig's base 0.255 mL (1.46 mmol), followed by the addition of methyl(2S)-amino(cyclohexyl)ethanoate hydrochloride 0.101 g (0.488 mmol) and the contents stirred at r.t. for 16 h. The crude reaction mixture was loaded onto an isco column and eluted with a gradient of EtOAc/Hex (0-60%) over 35 min to afford 0.20 g (71%) of a yellow solid.

### Step 2

### (2S)-cyclohexyl({[2-({[(2,6-dichlorophenyl)amino]carbonyl}amino)-5-(4-fluorophenyl)-3-thienyl]carbonyl}amino)ethanoic acid

To methyl(2S)-cyclohexyl({[2-({[(2,6-dichlorophenyl)amino]carbonyl}amino)-5-(4-fluorophenyl)-3-thienyl]carbonyl}amino)ethanoate 0.210 g (0.365 mmol) was added a 1.0 M solution of lithium hydroxide 1.83 mL (1.83 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.10 g (49%) as a yellow solid. ES MS m/z 564 (M+H).

### Example 420

### 2-({[(2,6-dichlorophenyl)amino]carbonyl}amino)-5-(4-fluoro-2-methylphenyl)-3-thiophenecarboxylic acid

### Step 1

### 1,1-dimethylethyl 2-({[(2,6-dichlorophenyl)amino]carbonyl}amino)-5-(4-fluoro-2-methylphenyl)-3-thiophenecarboxylate

To a solution of 1,1-dimethylethyl 5-bromo-2-({[(2,6-dichlorophenyl)amino]carbonyl}amino)-3-thiophenecarboxylate 0.4 g (0.860 mmol) in DME (7 mL) was added (4-fluoro-2-methylphenyl)boronic acid 0.198 g (1.29 mmol) followed by a solution of 2M Na₂CO₃ 1.72 mL and Dichlorobis(triphenylphosphine)palladium(II) 0.06 g (10 mole %) and the contents refluxed under nitrogen for 6 h. Filtration of the reaction mixture through celite followed by washing with EtOAc and concentration afforded the crude product which was loaded onto an isco column and eluted with EtOAc/Hexane (0-80%) to afford 0.251 g (59%) of a white solid.

### Step 2

### 2-({[(2,6-dichlorophenyl)amino]carbonyl}amino)-5-(4-fluoro-2-methylphenyl)-3-thiophenecarboxylic acid

To 1,1-dimethylethyl 2-({[(2,6-dichlorophenyl)amino]carbonyl}amino)-5-(4-fluoro-2-methylphenyl)-3-thiophenecarboxylate 0.190 g (0.384 mmol) was added TFA 0.3 mL (3.97 mmol) and chloroform (2.0 mL) and the contents heated at 50° C. for 2 h. Concentration of the reaction under vacuum afforded the desired product in quantative yield. ES MS m/z 439 (M+H).

### Example 421

### (2S)-cyclohexyl({[2-({[(2,6-dichlorophenyl)amino]carbonyl}amino)-5-(4-fluoro-2-methylphenyl)-3-thienyl]carbonyl}amino)ethanoic acid

### Step 1

### methyl(2S)-cyclohexyl({[2-({[(2,6-dichlorophenyl)amino]carbonyl}amino)-5-(4-fluoro-2-methylphenyl)-3-thienyl]carbonyl}amino)ethanoate

To 2-({[(2,6-dichlorophenyl)amino]carbonyl}amino)-5-[3-(methyloxy)phenyl]-3-thiophenecarboxylic acid 0.168 g (0.384 mmol) in DMF (3.0 mL) was added HATU 0.146 g (0.384 mmol) and Hunig's base 0.201 mL (1.15 mmol), followed by the addition of methyl(2S)-amino(cyclohexyl)ethanoate hydrochloride 0.080 g (0.384 mmol) and the contents stirred at r.t. for 16 h. The crude reaction mixture was loaded onto an isco column and eluted with a gradient of EtOAc/Hexane (0-60%) over 35 min to afford 0.20 g (88%) of a yellow solid.

### Step 2

### (2S)-cyclohexyl({[2-({[(2,6-dichlorophenyl)amino]carbonyl}amino)-5-(4-fluoro-2-methylphenyl)-3-thienyl]carbonyl}amino)ethanoic acid

To methyl(2S)-cyclohexyl({[2-({[(2,6-dichlorophenyl)amino]carbonyl}amino)-5-(4-fluoro-2-methylphenyl)-3-thienyl]carbonyl}amino)ethanoate 0.215 g (0.365 mmol) was added a 1.0 M solution of lithium hydroxide 1.83 mL (1.83 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded to desired product 0.127 g (61%) as a yellow solid. ES MS m/z 564 (M+H)

### Example 422

### 2-({[(2,6-dichlorophenyl)amino]carbonyl}amino)-5-(1-methyl-1H-pyrazol-4-yl)-3-thiophenecarboxylic acid

### Step 1

### 1,1-dimethylethyl 2-({[(2,6-dichlorophenyl)amino]carbonyl}amino)-5-(1-methyl-1H-pyrazol-4-yl)-3-thiophenecarboxylate

To a solution of 1,1-dimethylethyl 5-bromo-2-({[(2,6-dichlorophenyl)amino]carbonyl}amino)-3-thiophenecarboxylate 0.4 g (0.860 mmol) in DME (7 mL) was added 1-methyl-4-(4,4,5,5-tetramethyl-1,3,2-dioxaborolan-2-yl)-1H-pyrazole 0.268 g (1.29 mmol) followed by a solution of 2M Na₂CO₃ 1.72 mL and Dichlorobis(triphenylphosphine)palladium(l I) 0.06 g (10 mole %) and the contents refluxed under nitrogen for 6 h. Filtration of the reaction mixture through celite followed by washing with EtOAc and concentration afforded the crude product which was loaded onto an isco column and eluted with EtOAc/Hexane (0-80%) to afford 0.251 g (56%) of a white solid.

### Step 2

### 2-({[(2,6-dichlorophenyl)amino]carbonyl}amino)-5-(1-methyl-I H-pyrazol-4-yl)-3-thiophenecarboxylic acid

To 1,1-dimethylethyl 2-({[(2,6-dichlorophenyl)amino]carbonyl}amino)-5-(1-methyl-1H-pyrazol-4-yl)-3-thiophenecarboxylate 0.171 g (0.367 mmol) was added TFA 0.3 mL (3.97 mmol) and chloroform (2.0 mL) and the contents heated at 50° C. for 2 h. Concentration of the reaction under vacuum afforded the desired product in quantative yield. ES MS m/z 411 (M+H).

### Example 423

### (2S)-cyclohexyl({[2-({[(2,6-dichlorophenyl)amino]carbonyl}amino)-5-(1-methyl-1H-pyrazol-4-yl)-3-thienyl]carbonyl}amino)ethanoic acid

### Step 1

### methyl(2S)-cyclohexyl({[2-({[(2,6-dichlorophenyl)amino]carbonyl}amino)-5-(1-methyl-1H-pyrazol-4-yl)-3-thienyl]carbonyl}amino)ethanoate

To 2-({[(2,6-dichlorophenyl)amino]carbonyl}amino)-5-(1-methyl-1H-pyrazol-4-yl)-3-thiophenecarboxylic acid 0.150 g (0.365 mmol) in DMF (3.0 mL) was added HATU 0.138 g (0.365 mmol) and Hunig's base 0.254 mL (1.46 mmol), followed by the addition of methyl(2S)-amino(cyclohexyl)ethanoate hydrochloride 0.076 g (0.365 mmol) and the contents stirred at r.t. for 16 h. The crude reaction mixture was loaded onto an isco column and eluted with a gradient of EtOAc/Hexane (0-60%) over 35 min to afford 0.158 g (77%) of a yellow solid.

### Step 2

### (2S)-cyclohexyl({[2-({[(2,6-dichlorophenyl)amino]carbonyl}amino)-5-(1-methyl-1H-pyrazol-4-yl)-3-thienyl]carbonyl}amino)ethanoic acid

To methyl(2S)-cyclohexyl({[2-({[(2,6-dichlorophenyl)amino]carbonyl}amino)-5-(1-methyl-1H-pyrazol-4-yl)-3-thienyl]carbonyl}amino)ethanoate 0.158 g (0.280 mmol) was added a 1.0 M solution of lithium hydroxide 1.4 mL (1.40 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.129 g (84%) as a yellow solid. ES MS m/z 550 (M+H)

### Example 424

### 5-[4-(methyloxy)phenyl]-2-({[(2,4,6-trimethylphenyl)amino]carbonyl}amino)-3-thiophenecarboxylic acid

### Step 1

### 1,1-dimethylethyl 5-bromo-2-({[(2,4,6-trimethylphenyl)amino]carbonyl}amino)-3-thiophenecarboxylate

To a solution of 1,1-dimethylethyl 2-amino-5-bromo-3-thiophenecarboxylate 4.38 g (13.5 mmole) in DMF (20 mL) was added 2-isocyanato-1,3,5-trimethylbenzene 2.82 g (17.55 mmole) and triethylamine 3.78 mL (27 mmol) and the contents heated to 60° C. for 3 h. The crude mixture was loaded onto an isco column and eluted with EtOAc/Hexane (0-60%) to afford 6.48 g (88%) of a white solid.

### Step 2

### 1,1-dimethylethyl 5-[4-(methyloxy)phenyl]-2-({[(2,4,6-trimethylphenyl)amino]carbonyl}amino)-3-thiophenecarboxylate

1,1-dimethylethyl 5-bromo-2-({[(2,4,6-trimethylphenyl)amino]carbonyl}amino)-3-thiophenecarboxylate 0.319 g (0.728 mmol) in DME (5 mL) was added [4-(methyloxy)phenyl]boronic acid 0.143 g (0.946 mmol) followed by a solution of 2M Na₂CO₃ 1.46 mL and Dichlorobis(triphenylphosphine)palladium(II) 0.06 g (10 mole %) and the contents refluxed under nitrogen for 6 h. Filtration of the reaction mixture through celite followed by washing with EtOAc and concentration afforded the crude product which was loaded onto an isco column and eluted with EtOAc/Hexane (0-50%) to afford 0.128 g (38%) of a white solid.

### Step 3

### 5-[4-(methyloxy)phenyl]-2-({[(2,4,6-trimethylphenyl)amino]carbonyl}amino)-3-thiophenecarboxylic acid

To 1,1-dimethylethyl 5-[4-(methyloxy)phenyl]-2-({[(2,4,6-trimethylphenyl)amino]carbonyl}amino)-3-thiophenecarboxylate 0.128 g (0.274 mmol) was added TFA 1.0 mL (13 mmol) and chloroform (2.0 mL) and the contents heated at 50° C. for 2 h. Concentration of the reaction under vacuum afforded the desired product in quantative yield. ES MS m/z 411 (M+H).

### Example 425

### (2S)-cyclohexyl({[5-[4-(methyloxy)phenyl]-2-({[(2,4,6-trimethylphenyl)amino]carbonyl}amino)-3-thienyl]carbonyl}amino)ethanoic acid

### Step 1

### methyl(2S)-cyclohexyl({[5-[4-(methyloxy)phenyl]-2-({[(2,4,6-trimethylphenyl)amino]carbonyl}amino)-3-thienyl]carbonyl}amino)ethanoate

To 5-[4-(methyloxy)phenyl]-2-({[(2,4,6-trimethylphenyl)amino]carbonyl}amino)-3-thiophenecarboxylic acid 0.112 g (0.274 mmol) in DMF (3.0 mL) was added HATU 0.104 g (0.274 mmol) and Hunig's base 0.141 mL (0.822 mmol), followed by the addition of methyl(2S)-amino(cyclohexyl)ethanoate hydrochloride 0.057 g (0.274 mmol) and the contents stirred at r.t. for 16 h. The crude reaction mixture was loaded onto an isco column and eluted with a gradient of EtOAc/Hexane (0-60%) over 35 min to afford 0.120 g (78%) of a yellow solid.

### Step 2

### (2S)-cyclohexyl({[5-[4-(methyloxy)phenyl]-2-({[(2,4,6-trimethylphenyl)amino]carbonyl}amino)-3-thienyl]carbonyl}amino)ethanoic acid

To methyl(2S)-cyclohexyl({[5-[4-(methyloxy)phenyl]-2-({[(2,4,6-trimethylphenyl)amino]carbonyl}amino)-3-thienyl]carbonyl}amino)ethanoate 0.105 g (0.186 mmol) was added a 1.0 M solution of lithium hydroxide 0.746 mL (0.746 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.129 g (84%) as a yellow solid. ES MS m/z 550 (M+H).

### Example 426

### 2-{[({2,6-dichloro-4-[(trifluoromethyl)oxy]phenyl}amino)carbonyl]amino}-5-[4-(methyloxy)phenyl]-3-thiophenecarboxylic acid

### Step 1

### 1,1-dimethylethyl 5-bromo-2-{[({2,6-dichloro-4-[(trifluoromethyl)oxy]phenyl}amino)carbonyl]amino}-3-thiophenecarboxylate

To a solution of 1,1-dimethylethyl 2-amino-5-bromo-3-thiophenecarboxylate 2.5 g (8.99 mmole) in DMF (20 mL) was added 1,3-dichloro-2-isocyanato-5-[(trifluoromethyl)oxy]benzene 4.87 g (18.0 mmole) and triethylamine 2.53 mL (18.0 mmol) and the contents heated to 600° C. for 3 h. The crude mixture was loaded onto an isco column and eluted with EtOAc/Hexane (0-60%) to afford 3.1 g (79%) of a white solid.

### Step 2

### 1,1-dimethylethyl 2-{[({2,6-dichloro-4-[(trifluoromethyl)oxy]phenyl}amino)carbonyl]amino}-5-[4-(methyloxy)phenyl]-3-thiophenecarboxylate

To 1,1-dimethylethyl 5-bromo-2-{[({2,6-dichloro-4-[(trifluoromethyl)oxy]phenyl}amino)carbonyl]amino}-3-thiophenecarboxylate 0.40 g (0.728 mmol) in DME (5 mL) was added [4-(methyloxy)phenyl]boronic acid 0.143 g (0.946 mmol) followed by a solution of 2M Na₂CO₃ 1.46 mL and Dichlorobis(triphenylphosphine)palladium(II) 0.06 g (10 mole %) and the contents refluxed under nitrogen for 6 h. Filtration of the reaction mixture through celite followed by washing with EtOAc and concentration afforded the crude product which was loaded onto an isco column and eluted with EtOAc/Hexane (0-50%) to afford 0.278 g (66%) of a white solid.

### Step 3

### 2-{[({2,6-dichloro-4-[(trifluoromethyl)oxy]phenyl}amino)carbonyl]amino}-5-[4-(methyloxy)phenyl]-3-thiophenecarboxylic acid

To 1,1-dimethylethyl 2-{[({2,6-dichloro-4-[(trifluoromethyl)oxy]phenyl}amino)carbonyl]amino}-5-[4-(methyloxy)phenyl]-3-thiophenecarboxylate 0.278 g (0.482 mmol) was added TFA 1.0 mL (13 mmol) and chloroform (2.0 mL) and the contents heated at 500° C. for 2 h. Concentration of the reaction under vacuum afforded the desired product in quantative yield. ES MS m/z 521 (M+H).

### Example 427

### (2S)-cyclohexyl[({2-{[({2,6-dichloro-4-[(trifluoromethyl)oxy]phenyl}amino)carbonyl]amino}-5-[4-(methyloxy)phenyl]-3-thienyl}carbonyl)amino]ethanoic acid

### Step 1

### methyl(2S)-cyclohexyl[({2-{[({2,6-dichloro-4-[(trifluoromethyl)oxy]phenyl}amino)carbonyl]amino}-5-[4-(methyloxy)phenyl]-3-thienyl}carbonyl)amino]ethanoate

To 2-{[({2,6-dichloro-4-[(trifluoromethyl)oxy]phenyl}amino)carbonyl]amino}-5-[4-(methyloxy)phenyl]-3-thiophenecarboxylic acid 0.250 g (0.482 mmol) in DMF (3.0 mL) was added HATU 0.183 g (0.482 mmol) and Hunig's base 0.251 mL (1.45 mmol), followed by the addition of methyl(2S)-amino(cyclohexyl)ethanoate hydrochloride 0.100 g (0.482 mmol) and the contents stirred at r.t. for 16 h. The crude reaction mixture was loaded onto an isco column and eluted with a gradient of EtOAc/Hexane (0-60%) over 35 min to afford 0.189 g (58%) of a yellow solid.

### Step 2

### (2S)-cyclohexyl[({2-{[({2,6-dichloro-4-[(trifluoromethyl)oxy]phenyl}amino)carbonyl]amino}-5-[4-(methyloxy)phenyl]-3-thienyl}carbonyl)amino]ethanoic acid

To methyl(2S)-cyclohexyl[({2-{[({2,6-dichloro-4-[(trifluoromethyl)oxy]phenyl}amino)carbonyl]amino}-5-[4-(methyloxy)phenyl]-3-thienyl}carbonyl)amino]ethanoate 0.189 g (0.186 mmol) was added a 1.0 M solution of lithium hydroxide 1.12 mL (1.12 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.129 g (70%) as a yellow solid. ES MS m/z 660 (M+H).

### Example 428

### 2-{[({2,6-dichloro-4-[(trifluoromethyl)oxy]phenyl}amino)carbonyl]amino}-5-{4-[(trifluoromethyl)oxy]phenyl}-3-thiophenecarboxylic acid

### Step 1

### 1,1-dimethylethyl 2-{[({2,6-dichloro-4-[(trifluoromethyl)oxy]phenyl}amino)carbonyl]amino}-5-{4-[(trifluoromethyl)oxy]phenyl}-3-thiophenecarboxylate

To 1,1-dimethylethyl 5-bromo-2-{[({2,6-dichloro-4-[(trifluoromethyl)oxy]phenyl}amino)carbonyl]amino}-3-thiophenecarboxylate 0.40 g (0.728 mmol) in DME (5 mL) was added {4-[(trifluoromethyl)oxy]phenyl}boronic acid 0.194 g (0.946 mmol) followed by a solution of 2M Na₂CO₃ 1.46 mL and Dichlorobis(triphenylphosphine)palladium(II) 0.06 g (10 mole %) and the contents refluxed under nitrogen for 6 h. Filtration of the reaction mixture through celite followed by washing with EtOAc and concentration afforded the crude product which was loaded onto an isco column and eluted with EtOAc/Hexane (0-30%) to afford 0.182 g (40%) of a white solid.

### Step 2

### 2-{[({2,6-dichloro-4-[(trifluoromethyl)oxy]phenyl}amino)carbonyl]amino}-5-{4-[(trifluoromethyl)oxy]phenyl}-3-thiophenecarboxylic acid

To 1,1-dimethylethyl 2-{[({2,6-dichloro-4-[(trifluoromethyl)oxy]phenyl}amino)carbonyl]amino}-5-{4-[(trifluoromethyl)oxy]phenyl}-3-thiophenecarboxylate 0.182 g (0.288 mmol) was added TFA 1.0 mL (13.3 mmol) and chloroform (2.0 mL) and the contents heated at 50° C. for 2 h. Concentration of the reaction under vacuum afforded the desired product in quantative yield. ES MS m/z 575 (M+H).

### Example 429

### (2S)-cyclohexyl{[(2-{[({2,6-dichloro-4-[(trifluoromethyl)oxy]phenyl}amino)carbonyl]amino}-5-{4-[(trifluoromethyl)oxy]phenyl}-3-thienyl)carbonyl]amino}ethanoic acid

### Step 1

### methyl(2S)-cyclohexyl{[(2-{[({2,6-dichloro-4-[(trifluoromethyl)oxy]phenyl}amino)carbonyl]amino}-5-{4-[(trifluoromethyl)oxy]phenyl}-3-thienyl)carbonyl]amino}ethanoate

To 2-{[({2,6-dichloro-4-[(trifluoromethyl)oxy]phenyl}amino)carbonyl]amino}-5-{4-[(trifluoromethyl)oxy]phenyl}-3-thiophenecarboxylic acid 0.165 g (0.288 mmol) in DMF (3.0 mL) was added HATU 0.109 g (0.288 mmol) and Hunig's base 0.150 mL (0.864 mmol), followed by the addition of methyl(2S)-amino(cyclohexyl)ethanoate hydrochloride 0.060 g (0.288 mmol) and the contents stirred at r.t. for 16 h. The crude reaction mixture was loaded onto an isco column and eluted with a gradient of EtOAc/Hexane (0-40%) over 35 min to afford 0.085 g (41%) of a yellow solid.

### Step 2

### (2S)-cyclohexyl{[(2-{[({2,6-dichloro-4-[(trifluoromethyl)oxy]phenyl}amino)carbonyl]amino}-5-{4-[(trifluoromethyl)oxy]phenyl}-3-thienyl)carbonyl]amino}ethanoic acid

To methyl(2S)-cyclohexyl{[(2-{[({2,6-dichloro-4-[(trifluoromethyl)oxy]phenyl}amino)carbonyl]amino}-5-{4-[(trifluoromethyl)oxy]phenyl}-3-thienyl)carbonyl]amino}ethanoate 0.085 g (0.116 mmol) was added a 1.0 M solution of lithium hydroxide 0.467 mL (0.467 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.061 g (73%) as a yellow solid. ES MS m/z 714 (M+H).

### Example 430

### 5-{4-[(trifluoromethyl)oxy]phenyl}-2-({[(2,4,6-trimethylphenyl)amino]carbonyl}amino)-3-thiophenecarboxylic acid

### Step 1

### 1,1-dimethylethyl 5-{4-[(trifluoromethyl)oxy]phenyl}-2-({[(2,4,6-trimethylphenyl)amino]carbonyl}amino)-3-thiophenecarboxylate

1,1-dimethylethyl 5-bromo-2-({[(2,4,6-trimethylphenyl)amino]carbonyl}amino)-3-thiophenecarboxylate 0.319 g (0.728 mmol) in DME (5 mL) was added {4-[(trifluoromethyl)oxy]phenyl}boronic acid 0.194 g (0.946 mmol) followed by a solution of 2M Na₂CO₃ 1.46 mL and Dichlorobis(triphenylphosphine)palladium(II) 0.06 g (10 mole %) and the contents refluxed under nitrogen for 6 h. Filtration of the reaction mixture through celite followed by washing with EtOAc and concentration afforded the crude product which was loaded onto an isco column and eluted with EtOAc/Hexane (0-30%) to afford 0.086 g (23%) of a white solid.

### Step 2

### 5-{4-[(trifluoromethyl)oxy]phenyl}-2-({[(2,4,6-trimethylphenyl)amino]carbonyl}amino)-3-thiophenecarboxylic acid

To 1,1-dimethylethyl 5-{4-[(trifluoromethyl)oxy]phenyl}-2-({[(2,4,6-trimethylphenyl)amino]carbonyl}amino)-3-thiophenecarboxylate 0.080 g (0.153 mmol) was added TFA 1.0 mL (13.42 mmol) and chloroform (2.0 mL) and the contents heated at 500° C. for 2 h. Concentration of the reaction under vacuum afforded the desired product in quantative yield. ES MS m/z 465 (M+H).

### Example 431

### (2S)-cyclohexyl({[5-{4-[(trifluoromethyl)oxy]phenyl}-2-({[(2,4,6-trimethylphenyl)amino]carbonyl}amino)-3-thienyl]carbonyl}amino)ethanoic acid

### Step 1

### methyl(2S)-cyclohexyl({[5-{4-[(trifluoromethyl)oxy]phenyl}-2-({[(2,4,6-trimethylphenyl)amino]carbonyl}amino)-3-thienyl]carbonyl}amino)ethanoate

To 5-{4-[(trifluoromethyl)oxy]phenyl}-2-({[(2,4,6-trimethylphenyl)amino]carbonyl}amino)-3-thiophenecarboxylic acid 0.080 g (0.172 mmol) in DMF (3.0 mL) was added HATU 0.071 g (0.189 mmol) and Hunig's base 0.089 mL (0.516 mmol), followed by the addition of methyl(2S)-amino(cyclohexyl)ethanoate hydrochloride 0.040 g (0.189 mmol) and the contents stirred at r.t. for 16 h. The crude reaction mixture was loaded onto an isco column and eluted with a gradient of EtOAc/Hex (0-60%) over 35 min to afford 0.050 g (75%) of a yellow solid.

### Step 2

### (2S)-cyclohexyl({[5-{4-[(trifluoromethyl)oxy]phenyl}-2-({[(2,4,6-trimethylphenyl)amino]carbonyl}amino)-3-thienyl]carbonyl}amino)ethanoic acid

To methyl(2S)-cyclohexyl({[5-{4-[(trifluoromethyl)oxy]phenyl}-2-({[(2,4,6-trimethylphenyl)amino]carbonyl}amino)-3-thienyl]carbonyl}amino)ethanoate 0.050 g (0.081 mmol) was added a 1.0 M solution of lithium hydroxide 0.324 mL (0.324 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.042 g (87%) as a yellow solid. ES MS m/z 630 (M+H).

### Example 432

### 3-{[({2,6-dichloro-4-[(trifluoromethyl)oxy]phenyl}amino)carbonyl]amino}-5-[4-(methyloxy)phenyl]-2-thiophenecarboxylic acid

To a solution of 3-amino-5-(4-methoxyphenyl)-2-thiophenecarboxylic acid 2.52 g (10.15 mmol) in DMF (20 mL) was added 1,3-dichloro-2-isocyanato-5-[(trifluoromethyl)oxy]benzene 2.75 g (10.15 mmol) and triethylamine 1.85 mL (13.19 mmol) and the contents heated to 700° C. for 1.5 h. After addition of water, the reaction was acidified to pH=4 and the precipitated product filtered, washed with EtOAc and dried under vacuum to afford a yellow solid 3.1 g (71%). ES MS m/z 521 (M+H).

### Example 433

### (2S)-cyclohexyl[({3-{[({2,6-dichloro-4-[(trifluoromethyl)oxy]phenyl}amino)carbonyl]amino}-5-[4-(methyloxy)phenyl]-2-thienyl}carbonyl)amino]ethanoic acid

### Step 1

### methyl(2S)-cyclohexyl[({3-{[({2,6-dichloro-4-[(trifluoromethyl)oxy]phenyl}amino)carbonyl]amino}-5-[4-(methyloxy)phenyl]-2-thienyl}carbonyl)amino]ethanoate

To 3-{[({2,6-dichloro-4-[(trifluoromethyl)oxy]phenyl}amino)carbonyl]amino}-5-[4-(methyloxy)phenyl]-2-thiophenecarboxylic acid 0.248 g (0.478 mmol) in DMF (3.0 mL) was added HATU 0.219 g (0.573 mmol) and Hunig's base 0.085 mL (0.478 mmol), followed by the addition of methyl(2S)-amino(cyclohexyl)ethanoate hydrochloride 0.099 g (0.478 mmol) and the contents stirred at r.t. for 16 h. The crude reaction mixture was loaded onto an isco column and eluted with a gradient of EtOAc/Hexane (0-50%) over 40 min to afford 0.108 g (34%) of a yellow solid.

### Step 2

### (2S)-cyclohexyl[({3-{[({2,6-dichloro-4-[(trifluoromethyl)oxy]phenyl}amino)carbonyl]amino}-5-[4-(methyloxy)phenyl]-2-thienyl}carbonyl)amino]ethanoic acid

To methyl(2S)-cyclohexyl[({3-{[({2,6-dichloro-4-[(trifluoromethyl)oxy]phenyl}amino)carbonyl]amino}-5-[4-(methyloxy)phenyl]-2-thienyl}carbonyl)amino]ethanoate 0.108 g (0.160 mmol) was added a 1.0 M solution of lithium hydroxide 0.480 mL (0.480 mmol) and THF (1.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.091 g (87%) as a yellow solid. ES MS m/z 660 (M+H).

### Example 434

### 3-({[(2,6-dichlorophenyl)amino]carbonyl}amino)-5-{4-[(trifluoromethyl)oxy]phenyl}-2-thiophenecarboxylic acid

### Step 1

### (2E)-3-chloro-3-{4-[(trifluoromethyl)oxy]phenyl}-2-propenenitrile

To DMF (60 mL) cooled to 0° C. was added phosphorousoxychloride 6.72 mL (72.0 mmole) and the contents stirred for 10 mins. To the cooled reaction was added 1-{4-[(trifluoromethyl)oxy]phenyl}ethanone 5.88 g (72 mmole). After warming to r.t., the reaction mixture was heated at 50° C. for 10 mins. The reaction was then cooled to 0° C. and hydroxylamine hydrochloride 8.0 g (115.2 mole) was added slowly. After stirring for 5 mins., the contents were heated at 120° C. for 15 mins. After cooling to r.t., EtOAc was added followed by neutralization with satd. NaHCO₃. The organic layer was separated and the aqueous layer was extracted with EtOAc. The organic layer was dried, and then concentrated under vacuum to afford a brown oil that was carried on to the next step.

### Step 2

### methyl 3-amino-5-{4-[(trifluoromethyl)oxy]phenyl}-2-thiophenecarboxylate

To methanol (75 mL) was added a 25% solution of sodium methoxide in methanol 8.06 mL (0.037 mole) and methyl mercaptoacetate 2.58 mL (0.0288 mole) under nitrogen followed by the addition of (2E)-3-chloro-3-{4-[(trifluoromethyl)oxy]phenyl}-2-propenenitrile 7.42 g (0.0288 mole) in DMF (30 mL) at 0° C. After stirring for 30 mins., water was added to precipitate the desired product. After filtration and washing with water, the product was dried under vacuum to afford 6.4 g (67%).

### Step 3

### 3-amino-5-{4-[(trifluoromethyl)oxy]phenyl}-2-thiophenecarboxylic acid

To methyl 3-amino-5-{4-[(trifluoromethyl)oxy]phenyl}-2-thiophenecarboxylate 1.8 g (5.70 mmol) was added 1.0 M solution of lithium hydroxide 6.84 mL (6.84 mmol) and dioxane 10 mL. After refluxing for 2 h, the reaction was cooled and then acidified with 1N HCl. The precipitated product was filtered, dried and carried on to the next step.

### Step 4

### 3-({[(2,6-dichlorophenyl)amino]carbonyl}amino)-5-{4-[(trifluoromethyl)oxy]phenyl}-2-thiophenecarboxylic acid

To a solution of 3-amino-5-{4-[(trifluoromethyl)oxy]phenyl}-2-thiophenecarboxylic acid 0.250 g (0.825 mmol) in DMF (3.0 mL) was added 1,3-dichloro-2-isocyanatobenzene 0.186 g (0.99 mmol) and triethylamine 0.150 mL (1.07 mmol) and the contents heated to 70° C. for 1.5 h. After addition of water, the reaction was acidifies to pH=4 and the precipitated product filtered, washed with EtOAc and dried under vacuum to afford a yellow solid 0.31 g (77%). ES MS m/z 491 (M+H).

### Example 435

### (2S)-cyclohexyl{[(3-({[(2,6-dichlorophenyl)amino]carbonyl}amino)-5-{4-[(trifluoromethyl)oxy]phenyl}-2-thienyl)carbonyl]amino}ethanoic acid

### Step 1

### methyl(2S)-cyclohexyl{[(3-({[(2,6-dichlorophenyl)amino]carbonyl}amino)-5-{4-[(trifluoromethyl)oxy]phenyl}-2-thienyl)carbonyl]amino}ethanoate

To 3-({[(2,6-dichlorophenyl)amino]carbonyl}amino)-5-{4-[(trifluoromethyl)oxy]phenyl}-2-thiophenecarboxylic acid 0.207 g (0.422 mmol) in DMF (3.0 mL) was added HATU 0.176 g (0.464 mmol) and Hunig's base 0.220 mL (1.27 mmol), followed by the addition of methyl(2S)-amino(cyclohexyl)ethanoate hydrochloride 0.096 g (0.464 mmol) and the contents stirred at r.t. for 16 h. The crude reaction mixture was loaded onto an isco column and eluted with a gradient of EtOAc/Hex (0-50%) over 35 min to afford 0.210 g (77%) of a yellow solid.

### Step 2

### (2S)-cyclohexyl{[(3-({[(2,6-dichlorophenyl)amino]carbonyl}amino)-5-{4-[(trifluoromethyl)oxy]phenyl}-2-thienyl)carbonyl]amino}ethanoic acid

To methyl(2S)-cyclohexyl{[(3-({[(2,6-dichlorophenyl)amino]carbonyl}amino)-5-{4-[(trifluoromethyl)oxy]phenyl}-2-thienyl)carbonyl]amino}ethanoate 0.210 g (0.326 mmol) was added a 1.0 M solution of lithium hydroxide 0.979 mL (0.979 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.142 g (69%) as a yellow solid. ES MS m/z 630 (M+H).

### Example 436

### 5-{4-[(trifluoromethyl)oxy]phenyl}-3-({[(2,4,6-trimethylphenyl)amino]carbonyl}amino)-2-thiophenecarboxylic acid

To a solution of 3-amino-5-{4-[(trifluoromethyl)oxy]phenyl}-2-thiophenecarboxylic acid 0.250 g (0.825 mmol) in DMF (3.0 mL) was added 2-isocyanato-1,3,5-trimethylbenzene 0.159 g (0.99 mmol) and triethylamine 0.150 mL (1.07 mmol) and the contents heated to 700° C. for 1.5 h. After addition of water, the reaction was acidifies to pH=4 and the precipitated product filtered, washed with EtOAc and dried under vacuum to afford a yellow solid 0.294 g (77%). ES MS m/z 465 (M+H).

### Example 437

### (2S)-cyclohexyl({[5-{4-[(trifluoromethyl)oxy]phenyl}-3-({[(2,4,6-trimethylphenyl)amino]carbonyl}amino)-2-thienyl]carbonyl}amino)ethanoic acid

### Step 1

### methyl(2S)-cyclohexyl({[5-{4-[(trifluoromethyl)oxy]phenyl}-3-({[(2,4,6-trimethylphenyl)amino]carbonyl}amino)-2-thienyl]carbonyl}amino)ethanoate

To 5-{4-[(trifluoromethyl)oxy]phenyl}-3-({[(2,4,6-trimethylphenyl)amino]carbonyl}amino)-2-thiophenecarboxylic acid 0.191 g (0.411 mmol) in DMF (3.0 mL) was added HATU 0.171 g (0.452 mmol) and Hunig's base 0.215 mL (1.23 mmol), followed by the addition of methyl(2S)-amino(cyclohexyl)ethanoate hydrochloride 0.093 g (0.452 mmol) and the contents stirred at r.t. for 16 h. The crude reaction mixture was loaded onto an isco column and eluted with a gradient of EtOAc/Hexane (0-50%) over 35 min to afford 0.230 g (91%) of a yellow solid.

### Step 2

### (2S)-cyclohexyl({[5-{4-[(trifluoromethyl)oxy]phenyl}-3-({[(2,4,6-trimethylphenyl)amino]carbonyl}amino)-2-thienyl]carbonyl}amino)ethanoic acid

To methyl(2S)-cyclohexyl({[5-{4-[(trifluoromethyl)oxy]phenyl}-3-({[(2,4,6-trimethylphenyl)amino]carbonyl}amino)-2-thienyl]carbonyl}amino)ethanoate 0.230 g (0.372 mmol) was added a 1.0 M solution of lithium hydroxide 1.11 mL (1.11 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.184 g (82%) as a yellow solid. ES MS m/z 604 (M+H).

### Example 438

### 5-[4-(methyloxy)phenyl]-3-({[(2,4,6-trimethylphenyl)amino]carbonyl}amino)-2-thiophenecarboxylic acid

To a solution of 3-amino-5-(4-methoxyphenyl)-2-thiophenecarboxylic acid 4.0 g (16.0 mmol) in DMF (40 mL) was added 2-isocyanato-1,3,5-trimethylbenzene 2.84 g (17.6 mmol) and triethylamine 2.71 mL (19.2 mmol) and the contents heated to 70° C. for 2 h. After concentration of the reaction mixture, the contents were loaded onto an isco column eluting with EtOAc/Hexane (0-100%) to afford 1.9 g (29%) of a white solid. ES MS m/z 411 (M+H).

### Example 439

### (2S)-cyclohexyl({[5-[4-(methyloxy)phenyl]-3-({[(2,4,6-trimethylphenyl)amino]carbonyl}amino)-2-thienyl]carbonyl}amino)ethanoic acid

### Step 1

### methyl(2S)-cyclohexyl({[5-[4-(methyloxy)phenyl]-3-({[(2,4,6-trimethylphenyl)amino]carbonyl}amino)-2-thienyl]carbonyl}amino)ethanoate

To 5-[4-(methyloxy)phenyl]-3-({[(2,4,6-trimethylphenyl)amino]carbonyl}amino)-2-thiophenecarboxylic acid 0.150 g (0.344 mmol) in DMF (3.0 mL) was added HATU 0.143 g (0.378 mmol) and Hunig's base 0.179 mL (1.03 mmol), followed by the addition of methyl(2S)-amino(cyclohexyl)ethanoate hydrochloride 0.071 g (0.344 mmol) and the contents stirred at r.t. for 16 h. The crude reaction mixture was loaded onto an isco column and eluted with a gradient of EtOAc/Hexane (0-50%) over 35 min to afford 0.124 g (64%) of a yellow solid.

### Step 2

### (2S)-cyclohexyl({[5-[4-(methyloxy)phenyl]-3-({[(2,4,6-trimethylphenyl)amino]carbonyl}amino)-2-thienyl]carbonyl}amino)ethanoic acid

To methyl(2S)-cyclohexyl({[5-[4-(methyloxy)phenyl]-3-({[(2,4,6-trimethylphenyl)amino]carbonyl}amino)-2-thienyl]carbonyl}amino)ethanoate 0.10 g (0.178 mmol) was added a 1.0 M solution of lithium hydroxide 0.532 mL (0.532 mmol) and THF (0.5 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.093 g (96%) as a yellow solid. ES MS m/z 550 (M+H).

### Example 440

### (2S)-3-methyl-2-({[5-[4-(methyloxy)phenyl]-3-({[(2,4,6-trimethylphenyl)amino]carbonyl}amino)-2-thienyl]carbonyl}oxy)butanoic acid

### Step 1

### methyl N-{[5-[4-(methyloxy)phenyl]-3-({[(2,4,6-trimethylphenyl)amino]carbonyl}amino)-2-thienyl]carbonyl}-L-valinate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl L-valinate hydrochloride 0.122 g (0.731 mmol) and the contents stirred at r.t. for 16 h. The crude reaction mixture was loaded onto an isco column and eluted with a gradient of EtOAc/Hexane (0-50%) over 35 min to afford 0.20 g (78%) of a yellow solid.

### Step 2

### (2S)-3-methyl-2-({[5-[4-(methyloxy)phenyl]-3-({[(2,4,6-trimethylphenyl)amino]carbonyl}amino)-2-thienyl]carbonyl}oxy)butanoic acid

To methyl N-{[5-[4-(methyloxy)phenyl]-3-({[(2,4,6-trimethylphenyl)amino]carbonyl}amino)-2-thienyl]carbonyl}-L-valinate 0.20 g (0.382 mmol) was added a 1.0 M solution of lithium hydroxide 1.53 mL (1.53 mmol) and THF (3.5 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.168 g (86%) as a yellow solid. ES MS m/z 510 (M+H).

### Example 441

### N-{[5-[4-(methyloxy)phenyl]-3-({[(2,4,6-trimethylphenyl)amino]carbonyl}amino)-2-thienyl]carbonyl}-D-valine

### Step 1

### methyl N-{[5-[4-(methyloxy)phenyl]-3-({[(2,4,6-trimethylphenyl)amino]carbonyl}amino)-2-thienyl]carbonyl}-D-valinate

To 5-[4-(methyloxy)phenyl]-3-({[(2,4,6-trimethylphenyl)amino]carbonyl}amino)-2-thiophenecarboxylic acid 0.10 g (0.243 mmol) in DMF (2.0 mL) was added HATU 0.101 g (0.267 mmol) and Hunig's base 0.084 mL (0.486 mmol), followed by the addition of methyl D-valinate hydrochloride 0.045 g (0.267 mmol) and the contents stirred at r.t. for 16 h. The crude reaction mixture was loaded onto an isco column and eluted with a gradient of EtOAc/Hexane (0-60%) over 35 min to afford 0.067 g (53%) of a yellow oil.

### Step 2

### N-{[5-[4-(methyloxy)phenyl]-3-({[(2,4,6-trimethylphenyl)amino]carbonyl}amino)-2-thienyl]carbonyl}-D-valine

To methyl N-{[5-[4-(methyloxy)phenyl]-3-({[(2,4,6-trimethylphenyl)amino]carbonyl}amino)-2-thienyl]carbonyl}-D-valinate 0.067 g (0.128 mmol) was added a 1.0 M solution of lithium hydroxide 0.512 mL (0.512 mmol) and THF (1.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.051 g (78%) as a yellow solid. ES MS m/z 510 (M+H).

### Example 442

### N-{[5-[4-(methyloxy)phenyl]-3-({[(2,4,6-trimethylphenyl)amino]carbonyl}amino)-2-thienyl]carbonyl}-L-isoleucine

### Step 1

### methyl N-{[5-[4-(methyloxy)phenyl]-3-({[(2,4,6-trimethylphenyl)amino]carbonyl}amino)-2-thienyl]carbonyl}-L-isoleucinate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl L-isoleucinate hydrochloride 0.116 g (0.804 mmol) and the contents stirred at r.t. for 16 h. The crude reaction mixture was loaded onto an isco column and eluted with a gradient of EtOAc/Hexane (0-50%) over 35 min to afford 0.277 g (71%) of a yellow solid.

### Step 2

### N-{[5-[4-(methyloxy)phenyl]-3-({[(2,4,6-trimethylphenyl)amino]carbonyl}amino)-2-thienyl]carbonyl}-L-isoleucine

To methyl N-{[5-[4-(methyloxy)phenyl]-3-({[(2,4,6-trimethylphenyl)amino]carbonyl}amino)-2-thienyl]carbonyl}-L-isoleucinate 0.277 g (0.515 mmol) was added a 1.0 M solution of lithium hydroxide 2.06 mL (2.06 mmol) and THF (3.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.210 g (78%) as a yellow solid. ES MS m/z 524 (M+H).

### Example 443

### N-{[5-[4-(methyloxy)phenyl]-3-({[(2,4,6-trimethylphenyl)amino]carbonyl}amino)-2-thienyl]carbonyl}-L-norleucine

### Step 1

### methyl N-{[5-[4-(methyloxy)phenyl]-3-({[(2,4,6-trimethylphenyl)amino]carbonyl}amino)-2-thienyl]carbonyl}-L-norleucinate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl L-norleucinate hydrochloride 0.134 g (0.804 mmol) and the contents stirred at r.t. for 16 h. The crude reaction mixture was loaded onto an isco column and eluted with a gradient of EtOAc/Hexane (0-50%) over 35 min to afford 0.389 g (99%) of a yellow solid.

### Step 2

### N-{[5-[4-(methyloxy)phenyl]-3-({[(2,4,6-trimethylphenyl)amino]carbonyl}amino)-2-thienyl]carbonyl}-L-norleucine

To methyl N-{[5-[4-(methyloxy)phenyl]-3-({[(2,4,6-trimethylphenyl)amino]carbonyl}amino)-2-thienyl]carbonyl}-L-norleucinate 0.413 g (0.769 mmol) was added a 1.0 M solution of lithium hydroxide 3.84 mL (3.84 mmol) and THF (4.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.298 g (74%) as a yellow solid. ES MS m/z 524 (M+H).

### Example 444

### 3-cyclohexyl-N-{[5-[4-(methyloxy)phenyl]-3-({[(2,4,6-trimethylphenyl)amino]carbonyl}amino)-2-thienyl]carbonyl}-L-alanine

### Step 1

### methyl 3-cyclohexyl-N-{[5-[4-(methyloxy)phenyl]-3-({[(2,4,6-trimethylphenyl)amino]carbonyl}amino)-2-thienyl]carbonyl}-L-alaninate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl 3-cyclohexyl-L-alaninate hydrochloride 0.178 g (0.804 mmol) and the contents stirred at r.t. for 16 h. The crude reaction mixture was loaded onto an isco column and eluted with a gradient of EtOAc/Hexane (0-50%) over 35 min to afford 0.350 g (83%) of a yellow solid.

### Step 2

### 3-cyclohexyl-N-{[5-[4-(methyloxy)phenyl]-3-({[(2,4,6-trimethylphenyl)amino]carbonyl}amino)-2-thienyl]carbonyl}-L-alanine

To methyl 3-cyclohexyl-N-{[5-[4-(methyloxy)phenyl]-3-({[(2,4,6-trimethylphenyl)amino]carbonyl}amino)-2-thienyl]carbonyl}-L-alaninate 0.340 g (0.589 mmol) was added a 1.0 M solution of lithium hydroxide 1.5 mL (1.5 mmol) and THF (3.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.182 g (55%) as a yellow solid. ES MS m/z 564 (M+H).

### Example 445

### O-(1,1-dimethylethyl)-N-{[5-[4-(methyloxy)phenyl]-3-({[(2,4,6-trimethylphenyl)amino]carbonyl}amino)-2-thienyl]carbonyl}-L-serine

### Step 1

### methyl O-(1,1-dimethylethyl)-N-{[5-[4-(methyloxy)phenyl]-3-({[(2,4,6-trimethylphenyl)amino]carbonyl}amino)-2-thienyl]carbonyl}-L-serinate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl O-(1,1-dimethylethyl)-L-serinate hydrochloride 0.168 g (0.804 mmol) and the contents stirred at r.t. for 16 h. The crude reaction mixture was loaded onto an isco column and eluted with a gradient of EtOAc/Hexane (0-50%) over 35 min to afford 0.350 g (85%) of a yellow solid.

### Step 2

### O-(1,1-dimethylethyl)-N-{[5-[4-(methyloxy)phenyl]-3-({[(2,4,6-trimethylphenyl)amino]carbonyl}amino)-2-thienyl]carbonyl}-L-serine

To methyl O-(1,1-dimethylethyl)-N-{[5-[4-(methyloxy)phenyl]-3-({[(2,4,6-trimethylphenyl)amino]carbonyl}amino)-2-thienyl]carbonyl}-L-serinate 0.350 g (0.617 mmol) was added a 1.0 M solution of lithium hydroxide 2.46 mL (2.4 mmol) and THF (3.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.298 g (87%) as a yellow solid. ES MS m/z 554 (M+H).

### Example 446

### O-(1 μl-dimethylethyl)-N-{[5-[4-(methyloxy)phenyl]-3-({[(2,4,6-trimethylphenyl)amino]carbonyl}amino)-2-thienyl]carbonyl}-L-threonine

### Step 1

### methyl O-(1,1-dimethylethyl)-N-{[5-[4-(methyloxy)phenyl]-3-({[(2,4,6-trimethylphenyl)amino]carbonyl}amino)-2-thienyl]carbonyl}-L-threoninate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl O-(1,1-dimethylethyl)-L-threoninate hydrochloride 0.180 g (0.804 mmol) and the contents stirred at r.t. for 16 h. The crude reaction mixture was loaded onto an isco column and eluted with a gradient of EtOAc/Hexane (0-50%) over 35 min to afford 0.348 g (82%) of a yellow solid.

### Step 2

### O-(1,1-dimethylethyl)-N-{[5-[4-(methyloxy)phenyl]-3-({[(2,4,6-trimethylphenyl)amino]carbonyl}amino)-2-thienyl]carbonyl}-L-threonine

To methyl O-(1,1-dimethylethyl)-N-{[5-[4-(methyloxy)phenyl]-3-({[(2,4,6-trimethylphenyl)amino]carbonyl}amino)-2-thienyl]carbonyl}-L-threoninate 0.48 g (0.60 mmol) was added a 1.0 M solution of lithium hydroxide 2.40 mL (2.40 mmol) and THF (3.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.258 g (76%) as a yellow solid. ES MS m/z 568 (M+H).

### Example 447

### 1-{[5-[4-(methyloxy)phenyl]-3-({[(2,4,6-trimethylphenyl)amino]carbonyl}amino)-2-thienyl]carbonyl}-L-proline

### Step 1

### 1,1-dimethylethyl 1-{[5-[4-(methyloxy)phenyl]-3-({[(2,4,6-trimethylphenyl)amino]carbonyl}amino)-2-thienyl]carbonyl}-L-prolinate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of 1,1-dimethylethyl L-prolinate 0.125 g (0.731 mmol) and the contents stirred at r.t. for 16 h. The crude reaction mixture was loaded onto an isco column and eluted with a gradient of EtOAc/Hexane (0-60%) over 40 min to afford 0.301 g (73%) of a yellow solid.

### Step 2

### 1-{[5-[4-(methyloxy)phenyl]-3-({[(2,4,6-trimethylphenyl)amino]carbonyl}amino)-2-thienyl]carbonyl}-L-proline

To 1,1-dimethylethyl 1-{[5-[4-(methyloxy)phenyl]-3-({[(2,4,6-trimethylphenyl)amino]carbonyl}amino)-2-thienyl]carbonyl}-L-prolinate 0.301 g (0.534 mmol) was added TFA 1 mL (12.96 mmol) and chloroform (3 mL). The reaction mixture was refluxed for 1 h and then concentrated under vacuum to afford the product 0.268 g (99%) as a grey solid. ES MS m/z 508 (M+H).

### Example 448

### (2S)-1-{[5-[4-(methyloxy)phenyl]-3-({[(2,4,6-trimethylphenyl)amino]carbonyl}amino)-2-thienyl]carbonyl}-2-piperidinecarboxylic acid

### Step 1

### methyl(2S)-1-{[5-[4-(methyloxy)phenyl]-3-({[(2,4,6-trimethylphenyl)amino]carbonyl}amino)-2-thienyl]carbonyl}-2-piperidinecarboxylate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl(2S)-2-piperidinecarboxylate hydrochloride 0.131 g (0.731 mmol) and the contents stirred at r.t. for 16 h. The crude reaction mixture was loaded onto an isco column and eluted with a gradient of EtOAc/Hexane (0-50%) over 40 min to afford 0.312 g (80%) of a white solid.

### Step 2

### (2S)-1-{[5-[4-(methyloxy)phenyl]-3-({[(2,4,6-trimethylphenyl)amino]carbonyl}amino)-2-thienyl]carbonyl}-2-piperidinecarboxylic acid

To methyl(2S)-1-{[5-[4-(methyloxy)phenyl]-3-({[(2,4,6-trimethylphenyl)amino]carbonyl}amino)-2-thienyl]carbonyl}-2-piperidinecarboxylate 0.312 g (0.583 mmol) was added a 1.0 M solution of lithium hydroxide 1.75 mL (1.75 mmol) and THF (2.0 mL) and the contents stirred for 16 h. The reaction was then acidified to pH=4.0, the precipitated product was then extracted with EtOAc. Drying of the organic layer with magnesium sulfate followed by concentration under vacuum afforded the desired product 0.216 g (71%) as a yellow solid. ES MS m/z 522 (M+H).

### Example 449

### 1-({[5-[4-(methyloxy)phenyl]-3-({[(2,4,6-trimethylphenyl)amino]carbonyl}amino)-2-thienyl]carbonyl}amino)cyclopropanecarboxylic acid

### Step 1

### methyl 1-({[5-[4-(methyloxy)phenyl]-3-({[(2,4,6-trimethylphenyl)amino]carbonyl}amino)-2-thienyl]carbonyl}amino)cyclopropanecarboxylate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl 1-aminocyclopropanecarboxylate hydrochloride 0.110 g (0.731 mmol) and the contents stirred at r.t. for 16 h. The crude reaction mixture was loaded onto an isco column and eluted with a gradient of EtOAc/Hexane (0-50%) over 40 min to afford 0.217 g (59%) of a yellow solid.

### Step 2

### 1-({[5-[4-(methyloxy)phenyl]-3-({[(2,4,6-trimethylphenyl)amino]carbonyl}amino)-2-thienyl]carbonyl}amino)cyclopropanecarboxylic acid

To methyl 1-({[5-[4-(methyloxy)phenyl]-3-({[(2,4,6-trimethylphenyl)amino]carbonyl}amino)-2-thienyl]carbonyl}amino)cyclopropanecarboxylate 0.217 g (0.428 mmol) was added a 1.0 M solution of lithium hydroxide 2.14 mL(2.14 mmol) and dioxane (5.0 mL) and the contents refluxed for 2 h. The reaction was then acidified to pH=4.0, the precipitated product was then extracted with EtOAc. Drying of the organic layer with magnesium sulfate followed by concentration under vacuum afforded the desired product 0.178 g (84%) as a yellow solid. ES MS m/z 494 (M+H).

### Example 450

### 1-({[5-[4-(methyloxy)phenyl]-3-({[(2,4,6-trimethylphenyl)amino]carbonyl}amino)-2-thienyl]carbonyl}amino)cyclobutanecarboxylic acid

### Step 1

### methyl 1-({[5-[4-(methyloxy)phenyl]-3-({[(2,4,6-trimethylphenyl)amino]carbonyl}amino)-2-thienyl]carbonyl}amino)cyclobutanecarboxylate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl 1-aminocyclobutanecarboxylate 0.103 g (0.731 mmol) and the contents stirred at r.t. for 16 h. The crude reaction mixture was loaded onto an isco column and eluted with a gradient of EtOAc/Hexane (0-50%) over 40 min to afford 0.350 g (92%) of a yellow solid.

### Step 2

### 1-({[5-[4-(methyloxy)phenyl]-3-({[(2,4,6-trimethylphenyl)amino]carbonyl}amino)-2-thienyl]carbonyl}amino)cyclobutanecarboxylic acid

To methyl 1-({[5-[4-(methyloxy)phenyl]-3-({[(2,4,6-trimethylphenyl)amino]carbonyl}amino)-2-thienyl]carbonyl}amino)cyclobutanecarboxylate 0.349 g (0.671 mmol) was added a 1.0 M solution of lithium hydroxide 3.35 mL (3.35 mmol) and dioxane (5.0 mL) and the contents refluxed for 2 h. The reaction was then acidified to pH=4.0, the precipitated product was then extracted with EtOAc. Drying of the organic layer with magnesium sulfate followed by concentration under vacuum afforded the desired product 0.281 g (83%) as a yellow solid. ES MS m/z 508 (M+H).

### Example 451

### 1-({[5-[4-(methyloxy)phenyl]-3-({[(2,4,6-trimethylphenyl)amino]carbonyl}amino)-2-thienyl]carbonyl}amino)cyclopentanecarboxylic acid

### Step 1

### methyl 1-({[5-[4-(methyloxy)phenyl]-3-({[(2,4,6-trimethylphenyl)amino]carbonyl}amino)-2-thienyl]carbonyl}amino)cyclopentanecarboxylate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl 1-aminocyclopentanecarboxylate hydrochloride 0.143 g (0.804 mmol) and the contents stirred at r.t. for 16 h. The crude reaction mixture was loaded onto an isco column and eluted with a gradient of EtOAc/Hexane (0-60%) over 40 min to afford 0.350 g (90%) of a yellow solid.

### Step 2

### 1-({[5-[4-(methyloxy)phenyl]-3-({[(2,4,6-trimethylphenyl)amino]carbonyl}amino)-2-thienyl]carbonyl}amino)cyclopentanecarboxylic acid

To methyl 1-({[5-[4-(methyloxy)phenyl]-3-({[(2,4,6-trimethylphenyl)amino]carbonyl}amino)-2-thienyl]carbonyl}amino)cyclopentanecarboxylate 0.350 g (0.654 mmol) was added a 1.0 M solution of lithium hydroxide 3.27 mL (3.27 mmol) and dioxane (5.0 mL) and the contents refluxed for 2 h. The reaction was then acidified to pH=4.0, the precipitated product was then extracted with EtOAc. Drying of the organic layer with magnesium sulfate followed by concentration under vacuum afforded the desired product 0.294 g (87%) as a yellow solid. ES MS m/z 522 (M+H).

### Example 452

### 1-({[5-[4-(methyloxy)phenyl]-3-({[(2,4,6-trimethylphenyl)amino]carbonyl}amino)-2-thienyl]carbonyl}amino)cyclohexanecarboxylic acid

### Step 1

### methyl 1-({[5-[4-(methyloxy)phenyl]-3-({[(2,4,6-trimethylphenyl)amino]carbonyl}amino)-2-thienyl]carbonyl}amino)cyclohexanecarboxylate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl 1-aminocyclohexanecarboxylate 0.143 g (0.913 mmol) and the contents stirred at r.t. for 16 h. The crude reaction mixture was loaded onto an isco column and eluted with a gradient of EtOAc/Hexane (0-50%) over 35 min to afford 0.280 g (70%) of a yellow solid.

### Step 2

### 1-({[5-[4-(methyloxy)phenyl]-3-({[(2,4,6-trimethylphenyl)amino]carbonyl}amino)-2-thienyl]carbonyl}amino)cyclohexanecarboxylic acid

To methyl 1-({[5-[4-(methyloxy)phenyl]-3-({[(2,4,6-trimethylphenyl)amino]carbonyl}amino)-2-thienyl]carbonyl}amino)cyclohexanecarboxylate 0.280 g (0.510 mmol) was added a 1.0 M solution of lithium hydroxide 2.55 mL (2.55 mmol) and dioxane (5.0 mL) and the contents refluxed for 2 h. The reaction was then acidified to pH=4.0, the precipitated product was then extracted with EtOAc. Drying of the organic layer with magnesium sulfate followed by concentration under vacuum afforded the desired product 0.226 g (83%) as a yellow solid. ES MS m/z 536 (M+H).

### Example 453

### 1-({[5-[4-(methyloxy)phenyl]-3-({[(2,4,6-trimethylphenyl)amino]carbonyl}amino)-2-thienyl]carbonyl}amino)cycloheptanecarboxylic acid

### Step 1

### methyl 1-({[5-[4-(methyloxy)phenyl]-3-({[(2,4,6-trimethylphenyl)amino]carbonyl}amino)-2-thienyl]carbonyl}amino)cycloheptanecarboxylate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl 1-aminocycloheptanecarboxylate 0.155 g (0.913 mmol) and the contents stirred at r.t. for 16 h. The crude reaction mixture was loaded onto an isco column and eluted with a gradient of EtOAc/Hexane (0-50%) over 35 min to afford 0.282 g (69%) of a yellow solid.

### Step 2

### 1-({[5-[4-(methyloxy)phenyl]-3-({[(2,4,6-trimethylphenyl)amino]carbonyl}amino)-2-thienyl]carbonyl}amino)cycloheptanecarboxylic acid

To methyl 1-({[5-[4-(methyloxy)phenyl]-3-({[(2,4,6-trimethylphenyl)amino]carbonyl}amino)-2-thienyl]carbonyl}amino)cycloheptanecarboxylate 0.280 g (0.497 mmol) was added a 1.0 M solution of lithium hydroxide 2.48 mL (2.48 mmol) and dioxane (5.0 mL) and the contents refluxed for 2 h. The reaction was then acidified to pH=4.0, the precipitated product was then extracted with EtOAc. Drying of the organic layer with magnesium sulfate followed by concentration under vacuum afforded the desired product 0.210 g (77%) as a yellow solid. ES MS m/z 550 (M+H).

### Example 454

### 1-({[5-[4-(methyloxy)phenyl]-3-({[(2,4,6-trimethylphenyl)amino]carbonyl}amino)-2-thienyl]carbonyl}amino)cyclooctanecarboxylic acid

### Step 1

### methyl 1-({[5-[4-(methyloxy)phenyl]-3-({[(2,4,6-trimethylphenyl)amino]carbonyl}amino)-2-thienyl]carbonyl}amino)cyclooctanecarboxylate

To 5-[4-(methyloxy)phenyl]-3-({[(2,4,6-trimethylphenyl)amino]carbonyl}amino)-2-thiophenecarboxylic acid 0.30 g (0.731 mmol) in DMF (3.0 mL) was added HATU 0.305 g (0.804 mmol) and Hunig's base 0.255 mL (1.46 mmol), followed by the addition of methyl 1-aminocyclooctanecarboxylate 0.135 g (0.731 mmol) and the contents stirred at r.t. for 16 h. The crude reaction mixture was loaded onto an isco column and eluted with a gradient of EtOAc/Hexane (0-50%) over 40 min to afford 0.310 g (73%) of a yellow solid.

### Step 2

### 1-({[5-[4-(methyloxy)phenyl]-3-({[(2,4,6-trimethylphenyl)amino]carbonyl}amino)-2-thienyl]carbonyl}amino)cyclooctanecarboxylic acid

To methyl 1-({[5-[4-(methyloxy)phenyl]-3-({[(2,4,6-trimethylphenyl)amino]carbonyl}amino)-2-thienyl]carbonyl}amino)cyclooctanecarboxylate 0.30 g (0.519 mmol) was added a 1.0 M solution of lithium hydroxide 1.57 mL (1.57 mmol) and dioxane (3.0 mL) and the contents refluxed for 2 h. The reaction was then acidified to pH=4.0, the precipitated product was then extracted with EtOAc. Drying of the organic layer with magnesium sulfate followed by concentration under vacuum afforded the desired product 0.268 g (92%) as a yellow solid. ES MS m/z 564 (M+H).

### Example 455

### (2S)-cyclohexyl({[3-({[(2,6-dichloro-4-fluorophenyl)amino]carbonyl}amino)-2-naphthalenyl]carbonyl}amino)ethanoic acid

### Step 1

### methyl(2S)-cyclohexyl({[3-({[(2,6-dichloro-4-fluorophenyl)amino]carbonyl}amino)-2-naphthalenyl]carbonyl}amino)ethanoate

Methyl(2S)-[(3-amino-2-naphthoyl) amino](cyclohexyl)ethanoate hydrochloride (0.05 g, 0.133 mmol) in 5 mL of pyridine was treated 1,3-dichloro-5-fluoro-2-isocyanatobenzene (0.139 g, 0.67 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.051 g of product.

### Step 2

### (2S)-cyclohexyl({[3-({[(2,6-dichloro-4-fluorophenyl)amino]carbonyl}amino)-2-naphthalenyl]carbonyl}amino)ethanoic acid

Lithium hydroxide monohydrate (0.020 g, 0.48 mmol) was added to a solution of methyl(2S)-cyclohexyl({[3-({[(2,6-dichloro-4-fluorophenyl)amino]carbonyl}amino)-2-naphthalenyl]carbonyl}amino)ethanoate (0.050 g, 0.09 mmol) in THF:MeOH:water-3:1:1 mL. The mixture was stirred at RT overnight. The reaction mixture was acidified with 1N aqueous HCl and extracted with ethyl acetate. The organic phase was dried over sodium sulfate and concentrated to dryness to give 45 mg (92% yield) of desired product as a white solid. ES MS m/z 530 (M−H).

### Example 456

### 2-cyclohexyl-N-{[3-({[(2,4,6-trichlorophenyl)amino]carbonyl}amino)-2-naphthalenyl]carbonyl}-L-alanine

### Step 1

### methyl 2-cyclohexyl-N-{[(9H-fluoren-9-ylmethyl)oxy]carbonyl}-L-alaninate

To a solution of 2-cyclohexyl-N-{[(9H-fluoren-9-ylmethyl)oxy]carbonyl}-L-alanine (1.0 g, 2.54 mmol) in 25 mL of dichloromethane was added a dilute solution of diazomethane in methylene chloride until a yellow color remained. This was stirred for ca. 15 min and then acetic acid was added to remove the yellow color. The mixture was wash with aqueous NaHCO₃, dried over sodium sulfate and concentrated to dryness to give 1.1 g (100% yield) of desired product as a white solid.

### Step 2

### methyl 2-cyclohexyl-L-alaninate hydrochloride

To methyl 2-cyclohexyl-N-{[(9H-fluoren-9-ylmethyl)oxy]carbonyl}-L-alaninate (0.65 g, 1.60 mmol) in 20 mL of dioxane was added polymer bound piperizine (Aldrich Chemical catalog number 54,754-9, 5.3 g). The mixture was stirred at RT for 72 h, and then filtered and concentrated to dryness. To the crude product was added HCl in ether (1 M) followed by hexanes to give 0.205 g (57% yield) of the desired product as a white solid.

### Step 3

### methyl 2-cyclohexyl-N-{[3-({[(1,1-dimethylethyl)oxy]carbonyl}amino)-2-naphthalenyl]carbonyl}-L-alaninate

HATU (0.189 g, 0.50 mmol) was added to a solution of 3-[(tert-butoxycarbonyl)amino]-2-naphthoic acid (0.129 g, 0.45 mmol), methyl 2-cyclohexyl-L-alaninate hydrochloride (0.10 g, 0.45 mmol) and diisopropylethylamine (0.09 g, 0.68 mmol) in 5 mL of DMF. The mixture was stirred at RT for ca. 5 h. The DMF was removed under reduced pressure and the residue was diluted with ethyl acetate and water. The organic layer was washed with sodium hydrogen sulfate, sodium bicarbonate and brine, dried over sodium sulfate, filtered, and the solvent evaporated. Chromatography on silica gel with hexane/ethyl acetate gave 0.147 g (72% yield) of product.

### Step 4

### methyl N-[(3-amino-2-naphthalenyl)carbonyl]-2-cyclohexyl-L-alaninate hydrochloride

To methyl 2-cyclohexyl-N-{[3-({[(1,1-dimethylethyl)oxy]carbonyl}amino)-2-naphthalenyl]carbonyl}-L-alaninate (0.145 g, 0.318 mmol) in 10 mL of dichloromethane was added 5 mL of HCl in dioxane (4 N). The reaction was stirred at RT for ca. 3 h, and concentrated to dryness to give 0.139 g of the desire product.

### Step 5

### methyl 2-cyclohexyl-N-{[3-({[(2,4,6-trichlorophenyl)amino]carbonyl}amino)-2-naphthalenyl]carbonyl}-L-alaninate

Methyl N-[(3-amino-2-naphthalenyl)carbonyl]-2-cyclohexyl-L-alaninate hydrochloride (0.130 g, 0.33 mmol) in 10 mL of pyridine was treated 1,3,5-trichloro-2-isocyanatobenzene (0.37 g, 1.66 mmol) at RT for 6 h. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.15 g (80% yield) of product.

### Step 6

### 2-cyclohexyl-N-{[3-({[(2,4,6-trichlorophenyl)amino]carbonyl}amino)-2-naphthalenyl]carbonyl}-L-alanine

Lithium hydroxide monohydrate (0.095 g, 2.26 mmol) was added to a solution of methyl 2-cyclohexyl-N-{[3-({[(2,4,6-trichlorophenyl)amino]carbonyl}amino)-2-naphthalenyl]carbonyl}-L-alaninate (0.150 g, 0.26 mmol) in THF:MeOH: water-9:3:3 mL. The mixture was stirred at 70 C overnight. The reaction mixture was acidified with 1N aqueous HCl and extracted with ethyl acetate. The organic phase was dried over sodium sulfate and concentrated to dryness. Chromatography on silica gel with hexane/ethyl acetate gave to give 83 mg (55% yield) of desired product as a white solid. ES MS m/z 560 (M−H).

### Example 457

### (2S)-cyclohexyl{[(3-{[(2,4,6-trimethylphenyl)acetyl]amino}-2-naphthalenyl)carbonyl]amino}ethanoic acid

### Step 1

### methyl(2S)-cyclohexyl{[(3-{[(2,4,6-trimethylphenyl)acetyl]amino}-2-naphthalenyl)carbonyl]amino}ethanoate

HATU (0.14 g, 0.37 mmol) was added to a solution of methyl(2S)-[(3-amino-2-naphthoyl) amino](cyclohexyl)ethanoate hydrochloride (0.12 g, 0.32 mmol), (2,4,6-trimethylphenyl)acetic acid (0.062 g, 0.35 mmol), and diisopropylethylamine (0.062 g, 0.48 mmol) in 5 mL of DMF. The mixture was stirred at RT overnight. The DMF was removed under reduced pressure and the residue was diluted with ethyl acetate. The organic layer was washed with sodium bicarbonate and brine, dried over sodium sulfate, filtered, and concentrated to dryness. Chromatography on silica gel with hexane/ethyl acetate gave 0.072 g (45% yield) of product.

### Step 2

### (2S)-cyclohexyl{[(3-{[(2,4,6-trimethylphenyl)acetyl]amino}-2-naphthalenyl)carbonyl]amino}ethanoic acid

Lithium hydroxide monohydrate (0.025 g, 0.60 mmol) was added to a solution of methyl(2S)-cyclohexyl{[(3-{[(2,4,6-trimethylphenyl)acetyl]amino}-2-naphthalenyl)carbonyl]amino}ethanoate (0.068 g, 0.14 mmol) in THF:MeOH: water-3:1:1 mL. The mixture was stirred at RT overnight. The reaction mixture was acidified with 1N aqueous HCl and extracted with ethyl acetate. The organic phase was dried over sodium sulfate and concentrated to dryness to give 57 mg (86% yield) of desired product as a white solid. ES MS m/z 485 (M−H).

### Example 458

### (2S)-({[3-({[(4-bromo-2,6-dimethylphenyl)amino]carbonyl}amino)-2-naphthalenyl]carbonyl}amino)(cyclohexyl)ethanoic acid

### Step 1

### methyl(2S)-({[3-({[(4-bromo-2,6-dimethylphenyl)amino]carbonyl}amino)-2-naphthalenyl]carbonyl}amino)(cyclohexyl)ethanoate (u2208712611)

Methyl(2S)-[(3-amino-2-naphthoyl) amino](cyclohexyl)ethanoate hydrochloride (0.11 g, 0.29 mmol) in 10 mL of pyridine was treated 5-bromo-2-isocyanato-1,3-dimethylbenzene (0.139 g, 0.67 mmol) for 5 h at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.115 g (70% yield) of product.

### Step 2

### (2S)-({[3-({[(4-bromo-2,6-dimethylphenyl)amino]carbonyl}amino)-2-naphthalenyl]carbonyl}amino)(cyclohexyl)ethanoic acid

Lithium hydroxide monohydrate (0.017 g, 0.40 mmol) was added to a solution of methyl(2S)-({[3-({[(4-bromo-2,6-dimethylphenyl)amino]carbonyl}amino)-2-naphthalenyl]carbonyl}amino)(cyclohexyl)ethanoate (0.020 g, 0.035 mmol) in THF:MeOH:water-3:1:1 mL. The mixture was stirred at RT overnight. The reaction mixture was acidified with 1N aqueous HCl and extracted with ethyl acetate. The organic phase was dried over sodium sulfate and concentrated to dryness to give 19 mg (97% yield) of desired product as a white solid. ES MS m/z 550 (M−H).

### Example 459

### (2S)-cyclohexyl{[(3-{[(2,3,6-trichlorophenyl)acetyl]amino}-2-naphthalenyl)carbonyl]amino}ethanoic acid

### Step 1

### methyl(2S)-cyclohexyl{[(3-{[(2,3,6-trichlorophenyl)acetyl]amino}-2-naphthalenyl)carbonyl]amino}ethanoate

HATU (0.12 g, 0.31 mmol) was added to a solution of methyl(2S)-[(3-amino-2-naphthoyl) amino](cyclohexyl)ethanoate hydrochloride (0.100 g, 0.27 mmol), (2,3,6-trichlorophenyl)acetic acid (0.070 g, 0.29 mmol), and diisopropylethylamine (0.069 g, 0.53 mmol) in 6 mL of DMF. The mixture was stirred at RT overnight. The DMF was removed under reduced pressure and the residue was diluted with ethyl acetate. The organic layer was washed with sodium bicarbonate and brine, dried over sodium sulfate, filtered, and concentrated to dryness. Chromatography on silica gel with hexane/ethyl acetate gave 0.068 g (45% yield) of product.

### Step 2

### (2S)-cyclohexyl{[(3-{[(2,3,6-trichlorophenyl)acetyl]amino}-2-naphthalenyl)carbonyl]amino}ethanoic acid

Lithium hydroxide monohydrate (0.030 g, 0.71 mmol) was added to a solution of methyl(2S)-cyclohexyl{[(3-{[(2,3,6-trichlorophenyl)acetyl]amino}-2-naphthalenyl)carbonyl]amino}ethanoate (0.065 g, 0.12 mmol) in THF:MeOH: water-5:1.5:1.5 mL. The mixture was stirred at RT overnight. The reaction mixture was acidified with 1N aqueous HCl and extracted with ethyl acetate. The organic phase was dried over sodium sulfate and concentrated to dryness to give 56 mg (88% yield) of desired product as a white solid. ES MS m/z 545 (M−H).

### Example 460

### (2S)-cyclohexyl({[3-({[(4-ethyl-2,6-dimethylphenyl)amino]carbonyl}amino)-2-naphthalenyl]carbonyl}amino)ethanoic acid

### Step 1

### methyl(2S)-cyclohexyl({[3-({[(4-ethenyl-2,6-dimethylphenyl)amino]carbonyl}amino)-2-naphthalenyl]carbonyl}amino)ethanoate

Tetrakis(triphenylphosphine)palladium(0) (0.012 g, 0.01 mmol) was added to methyl(2S)-({[3-({[(4-bromo-2,6-dimethylphenyl)amino]carbonyl}amino)-2-naphthalenyl]carbonyl}amino)(cyclohexyl)ethanoate (0.088 g, 0.16 mmol) and tributyl(ethenyl)stannane (0.056 g, 0.18 mmol) in ca. 4 mL of toluene. The mixture was heated at reflux overnight. The solvent was removed under reduced pressure and chromatography on silica gel with hexane/ethyl acetate gave 0.046 g (56% yield) of product.

### Step 2

### methyl(2S)-cyclohexyl({[3-({[(4-ethyl-2,6-dimethylphenyl)amino]carbonyl}amino)-2-naphthalenyl]carbonyl}amino)ethanoate

A mixture of methyl(2S)-cyclohexyl({[3-({[(4-ethenyl-2,6-dimethylphenyl)amino]carbonyl}amino)-2-naphthalenyl]carbonyl}amino)ethanoate (0.046 g, 0.09 mmol) and palladium (10% on carbon, 0.04 g) in ca. 6 mL of ethyl acetate was stirred under 1 atm of hydrogen overnight. The mixture was flushed with nitrogen, filtered, and the solvent evaporated. Chromatography on silica gel with hexane/ethyl acetate gave 0.038 g (83% yield) of product.

### Step 3

### (2S)-cyclohexyl({[3-({[(4-ethyl-2,6-dimethylphenyl)amino]carbonyl}amino)-2-naphthalenyl]carbonyl}amino)ethanoic acid

Lithium hydroxide monohydrate (0.020 g, 0.48 mmol) was added to a solution of methyl(2S)-cyclohexyl({[3-({[(4-ethyl-2,6-dimethylphenyl)amino]carbonyl}amino)-2-naphthalenyl]carbonyl}amino)ethanoate (0.032 g, 0.06 mmol) in THF:MeOH: water-3:1:1 mL. The mixture was stirred at RT overnight. The reaction mixture was acidified with 1N aqueous HCl and extracted with ethyl acetate. The organic phase was dried over sodium sulfate and concentrated to dryness. Crystallization from ethyl acetate and hexanes gave 22 mg (71% yield) of desired product as a white solid. ES MS m/z 500 (M−H).

### Example 461

### (2S)-cyclohexyl{[(3-{[({2,6-dichloro-4-[(trifluoromethyl)oxy]phenyl}amino)carbonyl]amino}-2-naphthalenyl)carbonyl]amino}ethanoic acid

### Step 1

### Methyl(2S)-({3-[(tert-butoxycarbonyl)amino]-2-naphthoyl}amino)(cyclohexyl)ethanoate

HATU (0.875 g, 2.30 mmol) was added to a solution of 3-[(tert-butoxycarbonyl)amino]-2-naphthoic acid (0.575 g, 2.00 mmol), methyl(2S)-amino(cyclohexyl)ethanoate hydrochloride (0.479 g, 2.30 mmol) and diisopropylethylamine (0.387 g, 3.00 mmol) in 20 mL of DMF. The mixture was stirred at RT overnight. The DMF was removed under reduced pressure and the residue was diluted with ethyl acetate. The organic layer was washed with sodium hydrogensulfate, sodium bicarbonate, and brine, dried over sodium sulfate, and the solvent evaporated. Chromatography on silica gel with hexane/ethyl acetate gave 0.715 g of product.

### Step 2

### Methyl(2S)-[(3-amino-2-naphthoyl)amino](cyclohexyl)ethanoate hydrochloride

Methyl(2S)-({3-[(tert-butoxycarbonyl)amino]-2-naphthoyl}amino)(cyclohexyl)ethanoate (0.700 g, 1.0 mmol) in 20 mL of CH₂Cl₂ was treated with 20 mL of 4 N HCl in dioxane. The mixture as stirred at RT for ca. 3 h and the solvents were removed under reduced pressure to give the 0.675 g of the product.

### Step 3

### 1,3-dichloro-2-isocyanato-5-[(trifluoromethyl)oxy]benzene

2,6-dichloro-4-[(trifluoromethyl)oxy]aniline (0.50 g, 2.03 mmol) in 8 mL of dichloromethane was added to a mixture of phosgene (20% solution in toluene, 4 g) and PS-DIEA (Argonaut Technologies, 2.1 g, 3.9 mmol/g) in 25 mL of dichloromethane. After stirring at RT overnight the mixture was filtered and concentrated to give 0.52 g (94% yield) of the product.

### Step 4

### methyl(2S)-cyclohexyl{[(3-{[({2,6-dichloro-4-[(trifluoromethyl)oxy]phenyl}amino)carbonyl]amino}-2-naphthalenyl)carbonyl]amino}ethanoate

Methyl(2S)-[(3-amino-2-naphthoyl) amino](cyclohexyl)ethanoate hydrochloride (0.100 g, 0.265 mmol) in 7 mL of pyridine was treated 1,3-dichloro-2-isocyanato-5-[(trifluoromethyl)oxy]benzene (0.360 g, 0.1.33 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.101 g (62% yield) of product.

### Step 5

### (2S)-cyclohexyl{[(3-{[({2,6-dichloro-4-[(trifluoromethyl)oxy]phenyl}amino)carbonyl]amino}-2-naphthalenyl)carbonyl]amino}ethanoic acid

Lithium hydroxide monohydrate (0.05 g, 0.1.19 mmol) was added to a solution of methyl(2S)-cyclohexyl{[(3-{[({2,6-dichloro-4-[(trifluoromethyl)oxy]phenyl}amino)carbonyl]amino}-2-naphthalenyl)carbonyl]amino}ethanoate (0.095 g, 0.155 mmol) in THF:MeOH: water-9:3:3 mL. The mixture was stirred at RT overnight. The reaction mixture was acidified with 1N aqueous HCl and extracted with ethyl acetate. The organic phase was dried over sodium sulfate and concentrated to dryness to give 73 mg (79% yield) of desired product as a white solid. ES MS m/z 596 (M−H).

### Example 462

### (2S)-(trans-4-methylcyclohexyl)({[3-({[(2,4,6-trichlorophenyl)amino]carbonyl}amino)-2-naphthalenyl]carbonyl}amino)ethanoic acid

### Step 1

### (trans-4-methylcyclohexyl)methanol

Trans-4-methylcyclohexanecarboxylic acid (1.00 g, 7.03 mmol) in 8 mL of THF was added to an ice cold solution of lithium aluminum hydride (1N in THF, 7 mL, 7 mmol). The mixture was stirred at RT for 2.5 h and then returned to an ice bath. To this mixture was then added in order dropwise, water (0.28 mL), 15% NaOH (0.28 mL), and water (0.80 mL). After stirring 10 min, sodium sulfate was added, and the mixture was diluted with ether and filtered. Removal of the solvent gave 0.958 g of the desired product.

### Step 2

### trans-4-methylcyclohexanecarbaldehyde

Dess-Martin periodinane (4.45 g, 10.5 mmol) was added to (trans-4-methylcyclohexyl)methanol (7.0 mmol) in 60 mL of dichloromethane in 2 portions. The reaction was stirred at RT for 2.5 h, then was washed with sodium bicarbonate and dried over sodium sulfate. The solvent was removed under reduced pressure and the residue was diluted with ether, filter to remove solids and concentrate to give the product as an oil (0.95 g).

### Step 3

### (S)-4-methyl-N-[(1E)-(trans-4-methylcyclohexyl)methylidene]benzenesulfinamide

A mixture of (S)-4-methylbenzenesulfinamide (0.30 g, 1.93 mmol), trans-4-methylcyclohexanecarbaldehyde (0.37 g, 2.90 mmol) and titanium (IV) ethoxide (1.32 g, 5.8 mmol) in 20 mL of dichloromethane was refluxed overnight. The reaction was cooled to RT and 15 mL of water was added slowly. The resulting mixture was diluted with dichloromethane and filtered through a pad of celite. The phases were separated and the dichloromethane phase was washed with water and brine, dried over sodium sulfate, filtered, and the solvent evaporated to give 0.475 g (93% yield) of the product.

### Step 4

### N—[(S)-cyano(trans-4-methylcyclohexyl)methyl]-(S)-4-methylbenzenesulfinamide

To diethylaluminum cyanide (1N in THF, 2.35 mL, 2.35 mmol) in 7 mL of THF at −78 C was added isopropyl alcohol (0.94 g, 1.57 mmol). The mixture was stirred at RT for 30 min. This mixture was then cannulated into a −78 C solution of (S)-4-methyl-N-[(1E)-(trans-4-methylcyclohexyl)methylidene]benzenesulfinamide (0.42 g, 1.57 mmol) in 20 mL of THF. The mixture was warmed to RT over 2 h and stirred at RT overnight. The mixture was cooled to −78 C and 10 mL of a saturated ammonium chloride solution was added and the mixture was warmed to RT. The mixture was filtered through celite and extract with ethyl acetate. The combined organics were washed with brine, dried over sodium sulfate, filtered, and the solvent evaporated. Chromatography on silica gel with hexane/ethyl acetate gave 0.275 g (62% yield) of product.

### Step 5

### (2S)-amino(trans-4-methylcyclohexyl)ethanenitrile hydrochloride

A mixture of N—[(S)-cyano(trans-4-methylcyclohexyl)methyl]-(S)-4-methylbenzenesulfinamide (0.20 g, 0.69 mmol), hydrogen chloride (4 N in dioxane, 10 mL, 40 mmol) in 10 mL of methanol was heated at reflux for 6 h. The mixture was cooled to RT and the solvent evaporated. To the residue was added 10 mL of water and sodium bicarbonate, and this was extracted with ethyl acetate, dried over sodium sulfate, filtered and concentrated. The residue was dissolved in dichloromethane and hydrogen chloride (4 N in dioxane, 5 mL) was added and the solvent was removed. The residue was washed with ether to give 0.132 g of the product.

### Step 6

### methyl(2S)-amino(trans-4-methylcyclohexyl)ethanoate hydrochloride

Hydrogen chloride gas was bubbled into a mixture of (2S)-amino(trans-4-methylcyclohexyl)ethanenitrile hydrochloride (0.128 g, 0.68 mmol) in 15 mL of methanol until saturated. The mixture was refluxed overnight. The solvent was removed and the residue dissolved in water. Sodium bicarbonate was added and the mixture was extracted with ethyl acetate, dried over sodium sulfate and the solvent removed. The resulting residue was dissolved in methanol (15 mL) and was saturated with hydrogen chloride gas, and the mixture was refluxed for 6 h. Removal of the solvent gave the product.

### Step 7

### methyl(2S)-({[3-({[(1,1-dimethylethyl)oxy]carbonyl}amino)-2-naphthalenyl]carbonyl}amino)(trans-4-methylcyclohexyl)ethanoate

HATU (0.219 g, 0.575 mmol) was added to a solution of 3-[(tert-butoxycarbonyl)amino]-2-naphthoic acid (0.15 g, 0.523 mmol), (2S)-amino(trans-4-methylcyclohexyl)ethanoate hydrochloride (0.116 g, 0.523 mmol) and diisopropylethylamine (0.101 g, 0.784 mmol) in 7 mL of DMF. The mixture was stirred at RT for ca. 4.5 h. The DMF was removed under reduced pressure and the residue was diluted with ethyl acetate. The organic layer was washed with sodium hydrogensulfate, sodium bicarbonate and brine, dried over sodium sulfate, filtered, and the solvent evaporated. Chromatography on silica gel with hexane/ethyl acetate gave 0.197 g of ca. 60% pure product.

### Step 8

### methyl(2S)-{[(3-amino-2-naphthalenyl)carbonyl]amino}(trans-4-methylcyclohexyl)ethanoate hydrochloride

Methyl(2S)-({[3-({[(1,1-dimethylethyl)oxy]carbonyl}amino)-2-naphthalenyl]carbonyl}amino)(trans-4-methylcyclohexyl)ethanoate (0.195 g, 1.0 mmol) in 6 mL of CH₂Cl₂ was treated with 6 mL of 4 N HCl in dioxane. The mixture as stirred at RT for ca. 2 h and the solvents were removed under reduced pressure to give the 0.190 g of the product.

### Step 9

### methyl(2S)-(trans-4-methylcyclohexyl)({[3-({[(2,4,6-trichlorophenyl)amino]carbonyl}amino)-2-naphthalenyl]carbonyl}amino)ethanoate

Methyl(2S)-{[(3-amino-2-naphthalenyl)carbonyl]amino}(trans-4-methylcyclohexyl)ethanoate hydrochloride (0.19 g, 0.53 mmol) in 10 mL of pyridine was treated 1,3,5-trichloro-2-isocyanatobenzene (0.49 g, 2.10 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.089 g of product.

### Step 10

### (2S)-(trans-4-methylcyclohexyl)({[3-({[(2,4,6-trichlorophenyl)amino]carbonyl}amino)-2-naphthalenyl]carbonyl}amino)ethanoic acid

Lithium hydroxide monohydrate (0.050 g, 1.19 mmol) was added to a solution of methyl(2S)-(trans-4-methylcyclohexyl)({[3-({[(2,4,6-trichlorophenyl)amino]carbonyl}amino)-2-naphthalenyl]carbonyl}amino)ethanoate (0.084 g, 0.145 mmol) in THF:MeOH: water-9:3:3 mL. The mixture was stirred at RT overnight. The reaction mixture was acidified with 1N aqueous HCl and extracted with ethyl acetate. The organic phase was dried over sodium sulfate and concentrated to dryness to give 0.081 g (99% yield) of desired product as a white solid. ES MS m/z 560 (M−H).

### Example 463

### (2S)-[trans-4-(1,1-dimethylethyl)cyclohexyl]({[3-({[(2,4,6-trichlorophenyl)amino]carbonyl}amino)-2-naphthalenyl]carbonyl}amino)ethanoic acid

### Step 1

### [trans-4-(1,1-dimethylethyl)cyclohexyl]methanol

Trans-4-(1,1-dimethylethyl)cyclohexanecarboxylic acid (1.00 g, 5.43 mmol) in 8 mL of THF was added to an ice cold solution of lithium aluminum hydride (1N in THF, 6 mL, 6 mmol). The mixture was stirred at RT for 2.5 h and then returned to an ice bath. To this mixture was then added in order dropwise, water (0.23 mL), 15% NaOH (0.23 mL), and water (0.69 mL). After stirring 10 min, sodium sulfate was added, and the mixture was diluted with ether and filtered. Removal of the solvent gave 0.953 g of the desired product.

### Step 2

### trans-4-(1,1-dimethylethyl)cyclohexanecarbaldehyde

Dess-Martin periodinane (3.45 g, 8.14 mmol) was added to [trans-4-(1,1-dimethylethyl)cyclohexyl]methanol (5.43 mmol) in 50 mL of dichloromethane in 3 portions. The reaction was stirred at RT for 2 h, then was washed with sodium bicarbonate and dried over sodium sulfate. The solvent was removed under reduced pressure and the residue was diluted with ether, filter to remove solids and concentrate to give the product as an oil (0.822 g).

### Step 3

### (S)—N-{(1E)-[trans-4-(1,1-dimethylethyl)cyclohexyl]methylidene}benzenesulfinamide

A mixture of (S)-4-methylbenzenesulfinamide (0.60 g, 3.87 mmol), trans-4-(1,1-dimethylethyl)cyclohexanecarbaldehyde (0.714 g, 4.25 mmol) and titanium (IV) ethoxide (2.65 g, 11.61 mmol) in 40 mL of dichloromethane was refluxed overnight. The reaction was cooled to RT and 50 mL of water was added slowly. The resulting mixture was diluted with dichloromethane and filtered through a pad of celite. The phases were separated and the dichloromethane phase was washed with water and brine, dried over sodium sulfate, filtered, and the solvent evaporated. Chromatography on silica gel with hexane/ethyl acetate gave 0.49 g (41% yield) of product.

### Step 4

### N-{(S)-cyano[trans-4-(1,1-dimethylethyl)cyclohexyl]methyl}benzenesulfinamide

To diethylaluminum cyanide (1N in THF, 2.37 mL, 2.37 mmol) in 7 mL of THF at −78 C was added isopropyl alcohol (0.95 g, 1.58 mmol). The mixture was stirred at RT for 30 min. This mixture was then cannulated into a −78 C solution of (S)—N-{(1E)-[trans-4-(1,1-dimethylethyl)cyclohexyl]methylidene}benzenesulfinamide (0.485 g, 1.58 mmol) in 20 mL of THF. The mixture was warmed to RT overnight. The mixture was cooled to −78 C and 10 mL of a saturated ammonium chloride solution was added and the mixture was warmed to RT. The mixture was filtered through celite and extract with ethyl acetate. The combined organics were washed with brine, dried over sodium sulfate, filtered, and the solvent evaporated. Chromatography on silica gel with hexane/ethyl acetate gave 0.428 g (81% yield) of product.

### Step 5

### (2S)-amino[trans-4-(1,1-dimethylethyl)cyclohexyl]ethanoic acid hydrochloride

Hydrogen chloride gas was bubbled into a solution of N-{(S)-cyano[trans-4-(1,1-dimethylethyl)cyclohexyl]methyl}benzenesulfinamide (0.42 g, 1.26 mmol) in 15 mL of methanol in a high pressure tube until the solution was saturated. The tube was sealed and heated at 100 C for 24 h. The tube was cooled to RT then on ice and the cap carefully removed. The solvent was removed and water and sodium bicarbonate was added and the mixture was extracted with ethyl acetate. The mixture was concentrated and 15 mL of hydrochloric acid (6 N) was added and the solution refluxed overnight. The mixture was cooled to RT and 30 mL of ether was added. The resulting solid was collect by filtration and dried under reduced pressure to give 0.159 g of the product.

### Step 6

### methyl(2S)-amino[trans-4-(1,1-dimethylethyl)cyclohexyl]ethanoate hydrochloride

(2S)-amino[trans-4-(1,1-dimethylethyl)cyclohexyl]ethanoic acid hydrochloride (0.155 g, 0.72 mmol), concentrated hydrochloric acid (0.8 mL) and 2,2-Dimethoxypropane (10 mL) was stirred at RT overnight. The solvent was removed under reduced pressure and methanol was added. The methanol was removed under reduced pressure, and this was repeated. The resulting solid was triturated with ether to give 0.153 g of the product.

### Step 7

### methyl(2S)-[trans-4-(1,1-dimethylethyl)cyclohexyl]({[3-({[(1,1-dimethylethyl)oxy]carbonyl}amino)-2-naphthalenyl]carbonyl}amino)ethanoate

HATU (0.259 g, 0.682 mmol) was added to a solution of 3-[(tert-butoxycarbonyl)amino]-2-naphthoic acid (0.163 g, 0.568 mmol), methyl(2S)-amino[trans-4-(1,1-dimethylethyl)cyclohexyl]ethanoate hydrochloride (0.150 g, 0.568 mmol) and diisopropylethylamine (0.147 g, 1.14 mmol) in 7 mL of DMF. The mixture was stirred at RT overnight. The DMF was removed under reduced pressure and the residue was diluted with ethyl acetate. The organic layer was washed with sodium hydrogensulfate, sodium bicarbonate and brine, dried over sodium sulfate, filtered, and the solvent evaporated. Chromatography on silica gel with hexane/ethyl acetate gave 0.139 g (49% yield) of product.

### Step 8

### methyl(2S)-{[(3-amino-2-naphthalenyl)carbonyl]amino}[trans-4-(1,1-dimethylethyl)cyclohexyl]ethanoate hydrochloride

Methyl(2S)-[trans-4-(1,1-dimethylethyl)cyclohexyl]({[3-({[(1,1-dimethylethyl)oxy]carbonyl}amino)-2-naphthalenyl]carbonyl}amino)ethanoate (0.135 g, 0.27 mmol) in 8 mL of CH₂Cl₂ was treated with 8 mL of 4 N HCl in dioxane. The mixture as stirred at RT for ca. 2.5 h and the solvents were removed under reduced pressure to give the 0.132 g (100%) of the product.

### Step 9

### methyl(2S)-[trans-4-(1,1-dimethylethyl)cyclohexyl]({[3-({[(2,4,6-trichlorophenyl)amino]carbonyl}amino)-2-naphthalenyl]carbonyl}amino)ethanoate

Methyl(2S)-{[(3-amino-2-naphthalenyl)carbonyl]amino}[trans-4-(1,1-dimethylethyl)cyclohexyl]ethanoate hydrochloride (0.131 g, 0.33 mmol) in 10 mL of pyridine was treated 1,3,5-trichloro-2-isocyanatobenzene (0.30 g, 1.32 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.120 g (59% yield) of product.

### Step 10

### (2S)-[trans-4-(1,1-dimethylethyl)cyclohexyl]({[3-({[(2,4,6-trichlorophenyl)amino]carbonyl}amino)-2-naphthalenyl]carbonyl}amino)ethanoic acid

Lithium hydroxide monohydrate (0.075 g, 1.79 mmol) was added to a solution of methyl(2S)-[trans-4-(1,1-dimethylethyl)cyclohexyl]({[3-({[(2,4,6-trichlorophenyl)amino]carbonyl}amino)-2-naphthalenyl]carbonyl}amino)ethanoate (0.115 g, 0.186 mmol) in THF:MeOH: water-12:4:4 mL. The mixture was stirred at RT overnight. The reaction mixture was acidified with 1N aqueous HCl and extracted with ethyl acetate. The organic phase was dried over sodium sulfate and concentrated to dryness to give 0.105 g (93% yield) of desired product as a white solid. ES MS m/z 602 (M−H).

### Example 464

### 2-cyclohexyl-N-{[3-({[(2,4,6-trimethylphenyl)amino]carbonyl}amino)-2-naphthalenyl]carbonyl}-L-alanine

### Step 1

### methyl 2-cyclohexyl-N-{[(9H-fluoren-9-ylmethyl)oxy]carbonyl}-L-alaninate

To a solution of 2-cyclohexyl-N-{[(9H-fluoren-9-ylmethyl)oxy]carbonyl}-L-alanine (1.0 g, 2.54 mmol) in 15 mL of ethyl acetate and 15 mL of methanol was added (trimethylsilyl)diazomethane (2.0 M in hexanes, 3.74 mL). This was stirred for ca. 30 min and concentrated to dryness to give 1.15 g of desired product as a tacky white solid.

### Step 2

### methyl 2-cyclohexyl-L-alaninate hydrochloride

To methyl 2-cyclohexyl-N-{[(9H-fluoren-9-ylmethyl)oxy]carbonyl}-L-alaninate (1.1 g, 2.73 mmol) in 25 mL of dioxane was added polymer bound piperizine (Aldrich Chemical catalog number 54,754-9, 5.0 g). The mixture was stirred at RT for 72 h, then at ca. 60 C for 18 h. The mixture was filtered and concentrated to dryness. The residue was dissolved in 20 mL of dichloromethane and 5 mL of hydrogen chloride (4 N in dioxane) was added. Remove solvent and crystallize from dichloromethane and hexanes to give 0.493 g (81% yield) of the product.

### Step 3

### methyl 2-cyclohexyl-N-{[3-({[(1,1-dimethylethyl)oxy]carbonyl}amino)-2-naphthalenyl]carbonyl}-L-alaninate

HATU (0.880 g, 2.32 mmol) was added to a solution of 3-[(tert-butoxycarbonyl)amino]-2-naphthoic acid (0.606 g, 2.11 mmol), methyl 2-cyclohexyl-L-alaninate hydrochloride (0.468 g, 2.11 mmol) and diisopropylethylamine (0.408 g, 3.16 mmol) in 20 mL of DMF. The mixture was stirred at RT overnight. The DMF was removed under reduced pressure and the residue was diluted with ethyl acetate. The organic layer was washed with sodium hydrogensulfate, sodium bicarbonate and brine and dried over sodium sulfate, filtered, and the solvent evaporated. Chromatography on silica gel with hexane/ethyl acetate gave 0.667 g (70% yield) of product.

### Step 4

### methyl N-[(3-amino-2-naphthalenyl)carbonyl]-2-cyclohexyl-L-alaninate hydrochloride

To methyl 2-cyclohexyl-N-{[3-({[(1,1-dimethylethyl)oxy]carbonyl}amino)-2-naphthalenyl]carbonyl}-L-alaninate (0.667 g, 1.47 mmol) in 20 mL of methylene chloride was added 10 mL of HCl in dioxane (4 N). The reaction was stirred at RT for ca. 3.5 h, and concentrated to dryness to give 0.575 g of the desire product.

### Step 5

### methyl 2-cyclohexyl-N-{[3-({[(2,4,6-trimethylphenyl)amino]carbonyl}amino)-2-naphthalenyl]carbonyl}-L-alaninate

Methyl N-[(3-amino-2-naphthalenyl)carbonyl]-2-cyclohexyl-L-alaninate hydrochloride (0.155 g, 0.397 mmol) in 7 mL of pyridine was treated 2-isocyanato-1,3,5-trimethylbenzene (0.30 g, 1.98 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.171 g (83% yield) of product.

### Step 6

### 2-cyclohexyl-N-{[3-({[(2,4,6-trimethylphenyl)amino]carbonyl}amino)-2-naphthalenyl]carbonyl}-L-alanine

Lithium hydroxide monohydrate (0.150 g, 3.6 mmol) was added to a solution of methyl 2-cyclohexyl-N-{[3-({[(2,4,6-trimethylphenyl)amino]carbonyl}amino)-2-naphthalenyl]carbonyl}-L-alaninate (0.170 g, 0.33 mmol) in THF:MeOH: water-15:5:5 mL. The mixture was stirred at RT overnight. The reaction mixture was acidified with 1N aqueous HCl and extracted with ethyl acetate. The organic phase was dried over sodium sulfate and concentrated to dryness to give 147 mg (88% yield) of desired product as a white solid. ES MS m/z 500 (M−H).

### Example 465

### 2-cyclohexyl-N-[(3-{[({2,6-dichloro-4-[(trifluoromethyl)oxy]phenyl}amino)carbonyl]amino}-2-naphthalenyl)carbonyl]-L-alanine

### Step 1

### 1,3-dichloro-2-isocyanato-5-[(trifluoromethyl)oxy]benzene

2,6-dichloro-4-[(trifluoromethyl)oxy]aniline (4.00 g, 16.26 mmol) in 20 mL of dichloromethane was added to a mixture of phosgene (20% solution in toluene, 20.1 g) and PS-DIEA (Argonaut Technologies, 10.4 g, 3.9 mmol/g) in 150 mL of dichloromethane. After stirring at RT overnight the mixture was filtered and concentrated to give 4.8 g of the product (some toluene remaining).

### Step 2

### methyl 2-cyclohexyl-N-[(3-{[({2,6-dichloro-4-[(trifluoromethyl)oxy]phenyl}amino)carbonyl]amino}-2-naphthalenyl)carbonyl]-L-alaninate

Methyl N-[(3-amino-2-naphthalenyl)carbonyl]-2-cyclohexyl-L-alaninate hydrochloride (0.20 g, 0.512 mmol) in 10 mL of pyridine was treated 1,3-dichloro-2-isocyanato-5-[(trifluoromethyl)oxy]benzene (0.70 g, 2.56 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.273 g (85% yield) of product.

### Step 3

### 2-cyclohexyl-N-[(3-{[({2,6-dichloro-4-[(trifluoromethyl)oxy]phenyl}amino)carbonyl]amino}-2-naphthalenyl)carbonyl]-L-alanine

Lithium hydroxide monohydrate (0.150 g, 3.57 mmol) was added to a solution of methyl 2-cyclohexyl-N-[(3-{[({2,6-dichloro-4-[(trifluoromethyl)oxy]phenyl}amino)carbonyl]amino}-2-naphthalenyl)carbonyl]-L-alaninate (0.270 g, 0.431 mmol) in THF:MeOH: water-I 5:5:5 mL. The mixture was stirred at RT overnight. The reaction mixture was acidified with 1N aqueous HCl and extracted with ethyl acetate. The organic phase was dried over sodium sulfate and concentrated to dryness. Chromatography on silica gel with hexane/ethyl acetate gave 0.164 g (62% yield) of product. ES MS m/z 610 (M−H).

### Example 466

### {[(3-{[({2,6-dichloro-4-[(trifluoromethyl)oxy]phenyl}amino)carbonyl]amino}-2-naphthalenyl)carbonyl]amino}[trans-4-(trifluoromethyl)cyclohexyl]acetic acid

### Step 1

### methyl ({[(phenylmethyl)oxy]carbonyl}amino)[4-(trifluoromethyl)cyclohexylidene]acetate

DBU was added to a mixture of 4-(trifluoromethyl)cyclohexanone (1.00 g, 6.02 mmol), and methyl[bis(methyloxy)phosphoryl]({[(phenylmethyl)oxy]carbonyl}amino)acetate (1.99 g, 6.02 mmol) in 25 mL of dichloromethane. The mixture was stirred at RT overnight, diluted with 20 mL of dichloromethane and was washed with 1N hydrochloric acid, sodium bicarbonate, and brine. The organic phase was dried over sodium sulfate and concentrated to dryness. Chromatography on silica gel with hexane/ethyl acetate gave 1.38 g (62% yield) of product.

### Step 2

### methyl amino[4-(trifluoromethyl)cyclohexyl]acetate

Methyl ({[(phenylmethyl)oxy]carbonyl}amino)[4-(trifluoromethyl)cyclohexylidene]acetate (1.35 g, 3.64 mmol) and Pd on carbon (10%, 1.0 g) in 75 mL of methanol was stirred at RT overnight under 40 psi of hydrogen. The mixture was flushed with nitrogen, filtered through celite, and concentrated to dryness to give 0.875 g (100% yield) of the product as a 2:1 mixture of isomers.

### Step 3

### methyl ({[3-({[(1,1-dimethylethyl)oxy]carbonyl}amino)-2-naphthalenyl]carbonyl}amino)[trans-4-(trifluoromethyl)cyclohexyl]acetate and methyl ({[3-({[(1,1-dimethylethyl)oxy]carbonyl}amino)-2-naphthalenyl]carbonyl}amino)[cis-4-(trifluoromethyl)cyclohexyl]acetate

HATU (0.456 g, 1.20 mmol) was added to a solution of 3-[(tert-butoxycarbonyl)amino]-2-naphthoic acid (0.313 g, 1.09 mmol), methyl amino[4-(trifluoromethyl)cyclohexyl]acetate (0.261 g, 1.09 mmol) and diisopropylethylamine (0.211 g, 1.64 mmol) in 15 mL of DMF. The mixture was stirred at RT overnight. The DMF was removed under reduced pressure and the residue was diluted with ethyl acetate. The organic layer was washed with sodium hydrogensulfate, sodium bicarbonate and brine, dried over sodium sulfate, filtered, and the solvent evaporated. Chromatography on silica gel with hexane/ethyl acetate gave 0.242 g of methyl ({[3-({[(1,1-dimethylethyl)oxy]carbonyl}amino)-2-naphthalenyl]carbonyl}amino)[trans-4-(trifluoromethyl)cyclohexyl]acetate and 0.100 g of methyl ({[3-({[(1,1-dimethylethyl)oxy]carbonyl}amino)-2-naphthalenyl]carbonyl}amino)[cis-4-(trifluoromethyl)cyclohexyl]acetate.

### Step 4

### methyl {[(3-amino-2-naphthalenyl)carbonyl]amino}[trans-4-(trifluoromethyl)cyclohexyl]acetate hydrochloride

Methyl ({[3-({[(1,1-dimethylethyl)oxy]carbonyl}amino)-2-naphthalenyl]carbonyl}amino)[trans-4-(trifluoromethyl)cyclohexyl]acetate (0.240 g, 0.472 mmol) in 20 mL of CH₂Cl₂ was treated with 15 mL of 4 N HCl in dioxane. The mixture as stirred at RT for ca. 3 h and the solvents were removed under reduced pressure to give the 0.243 g of the product.

### Step 5

### methyl {[(3-{[({2,6-dichloro-4-[(trifluoromethyl)oxy]phenyl}amino)carbonyl]amino}-2-naphthalenyl)carbonyl]amino}[trans-4-(trifluoromethyl)cyclohexyl]acetate

Methyl {[(3-amino-2-naphthalenyl)carbonyl]amino}[trans-4-(trifluoromethyl)cyclohexyl]acetate hydrochloride (0.472 mmol) in 10 mL of pyridine was treated 1,3-dichloro-2-isocyanato-5-[(trifluoromethyl)oxy]benzene (0.385 g, 1.42 mmol) overnight at RT. The pyridine was removed under reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.275 g (86% yield) of product.

### Step 6

### {[(3-{[({2,6-dichloro-4-[(trifluoromethyl)oxy]phenyl}amino)carbonyl]amino}-2-naphthalenyl)carbonyl]amino}[trans-4-(trifluoromethyl)cyclohexyl]acetic acid

Lithium hydroxide monohydrate (0.15 g, 3.57 mmol) was added to a solution of methyl {[(3-{[({2,6-dichloro-4-[(trifluoromethyl)oxy]phenyl}amino)carbonyl]amino}-2-naphthalenyl)carbonyl]amino}[trans-4-(trifluoromethyl)cyclohexyl]acetate (0.270 g, 0.4 mmol) in THF:MeOH: water-15:5:5 mL. The mixture was stirred at RT overnight. The reaction mixture was acidified with 1N aqueous HCl and extracted with ethyl acetate. The organic phase was dried over sodium sulfate and concentrated to dryness to give 0.265 g (99% yield) of desired product as a white solid. ES MS m/z 664 (M−H).

### Example 467

### {[(3-{[({2,6-dichloro-4-[(trifluoromethyl)oxy]phenyl}amino)carbonyl]amino}-2-naphthalenyl)carbonyl]amino}[cis-4-(trifluoromethyl)cyclohexyl]acetic acid

### Step 1

### methyl {[(3-amino-2-naphthalenyl)carbonyl]amino}[cis-4-(trifluoromethyl)cyclohexyl]acetate hydrochloride

Methyl ({[3-({[(1,1-dimethylethyl)oxy]carbonyl}amino)-2-naphthalenyl]carbonyl}amino)[cis-4-(trifluoromethyl)cyclohexyl]acetate (0.10 g, 0.197 mmol) in 15 mL of CH₂Cl₂ was treated with 10 mL of 4 N HCl in dioxane. The mixture as stirred at RT for ca. 4 h and the solvents were removed under reduced pressure to give the 0.09 g of the product.

### Step 2

### methyl {[(3-{[({2,6-dichloro-4-[(trifluoromethyl)oxy]phenyl}amino)carbonyl]amino}-2-naphthalenyl)carbonyl]amino}[cis-4-(trifluoromethyl)cyclohexyl]acetate

Methyl {[(3-amino-2-naphthalenyl)carbonyl]amino}[cis-4-(trifluoromethyl)cyclohexyl]acetate hydrochloride (0.197 mmol) in 5 mL of pyridine was treated with 1,3-dichloro-2-isocyanato-5-[(trifluoromethyl)oxy]benzene (0.165 g, 0.727 mmol) overnight at RT. The pyridine was removed under reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.123 g (92% yield) of product.

### Step 3

### {[(3-{[({2,6-dichloro-4-[(trifluoromethyl)oxy]phenyl}amino)carbonyl]amino}-2-naphthalenyl)carbonyl]amino}[cis-4-(trifluoromethyl)cyclohexyl]acetic acid

Lithium hydroxide monohydrate (0.075 g, 1.79 mmol) was added to a solution of methyl{[(3-{[({2,6-dichloro-4-[(trifluoromethyl)oxy]phenyl}amino)carbonyl]amino}-2-naphthalenyl)carbonyl]amino}[cis-4-(trifluoromethyl)cyclohexyl]acetate (0.118 g, 0.173 mmol) in THF:MeOH: water-9:3:3 mL. The mixture was stirred at RT overnight. The reaction mixture was acidified with 1N aqueous HCl and extracted with ethyl acetate. The organic phase was dried over sodium sulfate and concentrated to dryness to give 0.090 g (78% yield) of desired product as a white solid. ES MS m/z 664 (M−H).

### Example 468

### {[(3-{[({2,6-dichloro-4-[(trifluoromethyl)oxy]phenyl}amino)carbonyl]amino}-2-naphthalenyl)carbonyl]amino}(tetrahydro-2H-pyran-4-yl)acetic acid

### Step 1

### methyl ({[(phenylmethyl)oxy]carbonyl}amino)(tetrahydro-4H-pyran-4-ylidene)acetate

DBU (0.909 g, 5.98 mmol) was added to a mixture of tetrahydro-4H-pyran-4-one (0.498 g, 4.98 mmol), and methyl[bis(methyloxy)phosphoryl]({[(phenylmethyl)oxy]carbonyl}amino)acetate (1.647 g, 4.98 mmol) in 20 mL of dichloromethane. The mixture was stirred at RT overnight, diluted with 10 mL of dichloromethane and wash with 1N hydrochloric acid, sodium bicarbonate, and brine. The organic phase was dried over sodium sulfate and concentrated to dryness. Chromatography on silica gel with hexane/ethyl acetate gave 0.855 g (56% yield) of product.

### Step 2

### methyl amino(tetrahydro-2H-pyran-4-yl)acetate

methyl({[(phenylmethyl)oxy]carbonyl}amino)(tetrahydro-4H-pyran-4-ylidene)acetate (0.430 g, 1.40 mmol) and Pd on carbon (10%, 0.35 g) in 30 mL of methanol was stirred at RT overnight under 50 psi of hydrogen. The mixture was flushed with nitrogen, filtered through celite and concentrated to dryness to give 0.225 g (92% yield) of the product.

### Step 3

### ({[3-({[(1,1-dimethylethyl)oxy]carbonyl}amino)-2-naphthalenyl]carbonyl}amino)(tetrahydro-2H-pyran-4-yl)acetic acid

HATU (0.484 g, 1.27 mmol) was added to a solution of 3-[(tert-butoxycarbonyl)amino]-2-naphthoic acid (0.317 g, 1.11 mmol), methyl amino(tetrahydro-2H-pyran-4-yl)acetate (0.220 g, 1.27 mmol) and diisopropylethylamine (0.214 g, 1.66 mmol) in 12 mL of DMF. The mixture was stirred at RT overnight. The DMF was removed under reduced pressure and the residue was diluted with ethyl acetate and water. The organic layer was washed with water and brine, dried over sodium sulfate, filtered, and the solvent evaporated. Chromatography on silica gel with hexane/ethyl acetate gave 0.412 g (84% yield) of product.

### Step 4

### {[(3-amino-2-naphthalenyl)carbonyl]amino}(tetrahydro-2H-pyran-4-yl)acetic acid hydrochloride

({[3-({[(1,1-dimethylethyl)oxy]carbonyl}amino)-2-naphthalenyl]carbonyl}amino)(tetrahydro-2H-pyran-4-yl)acetic acid (0.410 g, 0.93 mmol) in 10 mL of CH₂Cl₂ was treated with 10 mL of 4 N HCl in dioxane. The mixture as stirred at RT for ca. 3 h and the solvents were removed under reduced pressure to give the 0.405 g of the product.

### Step 5

### methyl {[(3-{[({2,6-dichloro-4-[(trifluoromethyl)oxy]phenyl}amino)carbonyl]amino}-2-naphthalenyl)carbonyl]amino}(tetrahydro-2H-pyran-4-yl)acetate

{[(3-amino-2-naphthalenyl)carbonyl]amino}(tetrahydro-2H-pyran-4-yl)acetic acid hydrochloride (0.200 g, 0.528 mmol) in 12 mL of pyridine was treated 1,3-dichloro-2-isocyanato-5-[(trifluoromethyl)oxy]benzene (0.431 g, 1.58 mmol) at RT overnight. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.297 g (92% yield) of product.

### Step 6

### {[(3-{[({2,6-dichloro-4-[(trifluoromethyl)oxy]phenyl}amino)carbonyl]amino}-2-naphthalenyl)carbonyl]amino}(tetrahydro-2H-pyran-4-yl)acetic acid

Lithium hydroxide monohydrate (0.150 g, 3.57 mmol) was added to a solution of methyl {[(3-{[({2,6-dichloro-4-[(trifluoromethyl)oxy]phenyl}amino)carbonyl]amino}-2-naphthalenyl)carbonyl]amino}(tetrahydro-2H-pyran-4-yl)acetate (0.285 g, 0.464 mmol) in THF:MeOH: water-15:5:5 mL. The mixture was stirred at RT overnight. The reaction mixture was acidified with 1N aqueous HCl and extracted with ethyl acetate. The organic phase was dried over sodium sulfate and concentrated to dryness to give 262 mg (90% yield) of desired product as a white solid. ES MS m/z 558 (M−H).

### Example 469

### tetrahydro-2H-pyran-4-yl({[3-({[(2,4,6-trimethylphenyl)amino]carbonyl}amino)-2-naphthalenyl]carbonyl}amino)acetic acid

### Step 1

### methyl tetrahydro-2H-pyran-4-yl({[3-({[(2,4,6-trimethylphenyl)amino]carbonyl}amino)-2-naphthalenyl]carbonyl}amino)acetate

{[(3-amino-2-naphthalenyl)carbonyl]amino}(tetrahydro-2H-pyran-4-yl)acetic acid hydrochloride (0.200 g, 0.528 mmol) in 12 mL of pyridine was treated 2-isocyanato-1,3,5-trimethylbenzene (0.255 g, 1.58 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.210 g (79% yield) of product.

### Step 2

### tetrahydro-2H-pyran-4-yl({[3-({[(2,4,6-trimethylphenyl)amino]carbonyl}amino)-2-naphthalenyl]carbonyl}amino)acetic acid

Lithium hydroxide monohydrate (0.100 g, 2.38 mmol) was added to a solution of methyl tetrahydro-2H-pyran-4-yl({[3-({[(2,4,6-trimethylphenyl)amino]carbonyl}amino)-2-naphthalenyl]carbonyl}amino)acetate (0.205 g, 0.407 mmol) in THF:MeOH: water-12:4:4 mL. The mixture was stirred at RT overnight. The reaction mixture was acidified with 1N aqueous HCl and extracted with ethyl acetate. The organic phase was dried over sodium sulfate and concentrated to dryness to give 0.183 g (92% yield) of desired product as a white solid. ES MS m/z 488 (M−H).

### Example 470

### (2S)-cyclohexyl({[3-({[(2,6-dimethyl-4-propylphenyl)amino]carbonyl}amino)-2-naphthalenyl]carbonyl}amino)ethanoic acid

### Step 1

### methyl(2S)-cyclohexyl({[3-({[(1,1-dimethylethyl)oxy]carbonyl}amino)-2-naphthalenyl]carbonyl}amino)ethanoate

HATU (2.287 g, 6.01 mmol) was added to a solution of 3-[(tert-butoxycarbonyl)amino]-2-naphthoic acid (1.50 g, 5.23 mmol), methyl(2S)-amino(cyclohexyl)ethanoate hydrochloride (1.25 g, 1.56 mmol) and diisopropylethylamine (1.01 g, 7.84 mmol) in 50 mL of DMF. The mixture was stirred at RT for ca. 3 h. The DMF was removed under reduced pressure and the residue was diluted with ethyl acetate. The organic layer was washed with sodium hydrogensulfate, sodium bicarbonate and brine, dried over sodium sulfate, filtered, and the solvent evaporated. Chromatography on silica gel with hexane/ethyl acetate gave 1.73 g (75% yield) of product.

### Step 2

### methyl(2S)-{[(3-amino-2-naphthalenyl)carbonyl]amino}(cyclohexyl)ethanoate hydrochloride

Methyl(2S)-cyclohexyl({[3-({[(1,1-dimethylethyl)oxy]carbonyl}amino)-2-naphthalenyl]carbonyl}amino)ethanoate

(1.725 g, 3.92 mmol) in 50 mL of dichloromethane was treated with 35 mL of 4 N HCl in dioxane. The mixture as stirred at RT for ca. 4 h and the solvents were removed under reduced pressure to give the 1.51 g of the product.

### Step 3

### methyl(2S)-({[3-({[(4-bromo-2,6-dimethylphenyl)amino]carbonyl}amino)-2-naphthalenyl]carbonyl}amino)(cyclohexyl)ethanoate

Methyl(2S)-{[(3-amino-2-naphthalenyl)carbonyl]amino}(cyclohexyl)ethanoate hydrochloride (0.700 g, 1.86 mmol) in 40 mL of pyridine was treated 5-bromo-2-isocyanato-1,3-dimethylbenzene (1.05 g, 4.65 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.825 g (80% yield) of product.

### Step 4

### methyl(2S)-cyclohexyl[({3-[({[2,6-dimethyl-4-(2-propen-1-yl)phenyl]amino}carbonyl)amino]-2-naphthalenyl}carbonyl)amino]ethanoate

Tetrakis(triphenylphosphine)palladium(0) (0.025 g, 0.022 mmol) was added to methyl(2S)-({[3-({[(4-bromo-2,6-dimethylphenyl)amino]carbonyl}amino)-2-naphthalenyl]carbonyl}amino)(cyclohexyl)ethanoate (0.200 g, 0.363 mmol) and tributyl(2-propen-1-yl)stannane (0.132 g, 0.399 mmol) in ca. 10 mL of toluene. The mixture was heated at reflux overnight. The solvent was removed under reduced pressure and chromatography on silica gel with hexane/ethyl acetate gave 0.090 g (46% yield) of product.

### Step 5

### methyl(2S)-cyclohexyl({[3-({[(2,6-dimethyl-4-propylphenyl)amino]carbonyl}amino)-2-naphthalenyl]carbonyl}amino)ethanoate

A mixture of methyl(2S)-cyclohexyl[({3-[({[2,6-dimethyl-4-(2-propen-1-yl)phenyl]amino}carbonyl)amino]-2-naphthalenyl}carbonyl)amino]ethanoate (0.090 g, 0.171 mmol) and palladium (10% on carbon, 0.090 g) in ca. 15 mL of ethyl acetate was stirred under 50 psi of hydrogen for 4 h. The mixture was flushed with nitrogen, filtered, and the solvent evaporated to give 0.075 g (83% yield) of product.

### Step 6

### (2S)-cyclohexyl({[3-({[(2,6-dimethyl-4-propylphenyl)amino]carbonyl}amino)-2-naphthalenyl]carbonyl}amino)ethanoic acid

Lithium hydroxide monohydrate (0.065 g, 1.55 mmol) was added to a solution of methyl(2S)-cyclohexyl({[3-({[(2,6-dimethyl-4-propylphenyl)amino]carbonyl}amino)-2-naphthalenyl]carbonyl}amino)ethanoate (0.075 g, 0.14 mmol) in THF:MeOH: water-9:3:3 mL. The mixture was stirred at RT overnight. The reaction mixture was acidified with 1N aqueous HCl and extracted with ethyl acetate. The organic phase was dried over sodium sulfate and concentrated to dryness. Chromatography on silica gel with hexane/ethyl acetate gave 0.037 g (51% yield) of desired product as a white solid. ES MS m/z 514 (M−H).

### Example 471

### (2S)-cyclohexyl[({3-[({[2,6-dimethyl-4-(2-propyn-1-yl)phenyl]amino}carbonyl)amino]-2-naphthalenyl}carbonyl)amino]ethanoic acid

### Step 1

### methyl(2S)-cyclohexyl[({3-[({[2,6-dimethyl-4-(2-propyn-1-yl)phenyl]amino}carbonyl)amino]-2-naphthalenyl}carbonyl)amino]ethanoate

Tetrakis(triphenylphosphine)palladium(0) (0.013 g, 0.01 mmol) was added to methyl(2S)-({[3-({[(4-bromo-2,6-dimethylphenyl)amino]carbonyl}amino)-2-naphthalenyl]carbonyl}amino)(cyclohexyl)ethanoate (0.100 g, 0.181 mmol) and tributyl(2-propyn-1-yl)stannane (0.065 g, 0.199 mmol) in ca. 2.5 mL of acetonitrile. The mixture was heated in a microwave reactor at 150 C for 30 min. The solvent was removed under reduced pressure and chromatography on silica gel with hexane/ethyl acetate gave 0.039 g (41% yield) of product.

### Step 2

### (2S)-cyclohexyl[({3-[({[2,6-dimethyl-4-(2-propyn-1-yl)phenyl]amino}carbonyl)amino]-2-naphthalenyl}carbonyl)amino]ethanoic acid

Lithium hydroxide monohydrate (0.025 g, 0.60 mmol) was added to a solution of methyl(2S)-cyclohexyl[({3-[({[2,6-dimethyl-4-(2-propyn-1-yl)phenyl]amino}carbonyl)amino]-2-naphthalenyl}carbonyl)amino]ethanoate (0.038 g, 0.07 mmol) in THF:MeOH:water-3:1:1 mL. The mixture was stirred at RT overnight. The reaction mixture was acidified with 1N aqueous HCl and extracted with ethyl acetate. The organic phase was dried over sodium sulfate and concentrated to dryness to give 0.036 g (98% yield) of desired product as a white solid. ES MS m/z 510 (M−H).

### Example 472

### 2-cyclohexyl-N-{[4-fluoro-2-({[(2,4,6-trimethylphenyl)amino]carbonyl}amino)phenyl]carbonyl}-L-alanine

### Step 1

### Methyl 2-cyclohexyl-N-{[(9H-fluoren-9-ylmethyl)oxy]carbonyl}-L-alaninate

To a solution of 2-cyclohexyl-N-{[(9H-fluoren-9-ylmethyl)oxy]carbonyl}-L-alanine (1.0 g, 2.54 mmol) in 15 mL of ethyl acetate and 15 mL of methanol was added (trimethylsilyl)diazomethane (2.0 M in hexanes, 3.50 mL). This was stirred for ca. 45 min and concentrated to dryness to give 1.15 g of desired product as a tacky white solid.

### Step 2

### methyl 2-cyclohexyl-L-alaninate hydrochloride

To methyl 2-cyclohexyl-N-{[(9H-fluoren-9-ylmethyl)oxy]carbonyl}-L-alaninate (1.1 g, 2.73 mmol) in 25 mL of dioxane was added polymer bound piperizine (Aldrich Chemical catalog number 54,754-9, 5.0 g). The mixture was heated at 60 C for 36 h. The mixture was filtered and concentrated to dryness. The residue was dissolved in 20 mL of dichloromethane and 5 mL of hydrogen chloride (4 N in dioxane) was added. The solvent was removed and the residue was crystallize from dichloromethane and hexanes. The mother liquors were concentrated to dryness and triturated with ethyl acetate and hexanes. Solids were combined to give 0.545 g (90% yield) of the product.

### Step 3

### methyl 2-cyclohexyl-N-[(4-fluoro-2-nitrophenyl)carbonyl]-L-alaninate

HATU (0.514 g, 1.35 mmol) was added to a solution of 4-fluoro-2-nitrobenzoic acid (0.208 g, 1.13 mmol), methyl 2-cyclohexyl-L-alaninate hydrochloride (0.250 g, 1.13 mmol) and diisopropylethylamine (0.218 g, 1.69 mmol) in 10 mL of DMF. The mixture was stirred at RT overnight. The DMF was removed under reduced pressure and the residue was diluted with ethyl acetate. The organic layer was washed with sodium hydrogensulfate, sodium bicarbonate and brine, dried over sodium sulfate, filtered, and the solvent evaporated. Chromatography on silica gel with hexane/ethyl acetate gave 0.308 g (78% yield) of product.

### Step 4

### methyl N-[(2-amino-4-fluorophenyl)carbonyl]-2-cyclohexyl-L-alaninate

A mixture of methyl 2-cyclohexyl-N-[(4-fluoro-2-nitrophenyl)carbonyl]-L-alaninate (0.305 g, 0.866 mmol) and palladium (10% on carbon, 0.200 g) in ca. 15 mL of ethanol was stirred under 60 psi of hydrogen for 2 h. The mixture was flushed with nitrogen, filtered, and the solvent evaporated to give 0.219 g (78% yield) of product.

### Step 5

### methyl 2-cyclohexyl-N-{[4-fluoro-2-({[(2,4,6-trimethylphenyl)amino]carbonyl}amino)phenyl]carbonyl}-L-alaninate

Methyl N-[(2-amino-4-fluorophenyl)carbonyl]-2-cyclohexyl-L-alaninate (0.100 g, 0.310 mmol) in 6 mL of pyridine was treated 2-isocyanato-1,3,5-trimethylbenzene (0.151 g, 0.932 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.132 g (88% yield) of product.

### Step 6

### 2-cyclohexyl-N-{[4-fluoro-2-({[(2,4,6-trimethylphenyl)amino]carbonyl}amino)phenyl]carbonyl}-L-alanine

Lithium hydroxide monohydrate (0.075 g, 0.48 mmol) was added to a solution of methyl 2-cyclohexyl-N-{[4-fluoro-2-({[(2,4,6-trimethylphenyl)amino]carbonyl}amino)phenyl]carbonyl}-L-alaninate (0.130 g, 0.269 mmol) in THF:MeOH:water-9:3:3 mL. The mixture was stirred at RT overnight. The reaction mixture was acidified with 1N aqueous HCl and extracted with ethyl acetate. The organic phase was dried over sodium sulfate and concentrated to dryness to give 0.128 g (100% yield) of desired product as a white solid. ES MS m/z 468 (M−H).

### Example 473

### (2S)-cyclohexyl[({3-[({[2,6-dimethyl-4-(propyloxy)phenyl]amino}carbonyl)amino]-2-naphthalenyl}carbonyl)amino]ethanoic acid

### Step 1

### 3,5-dimethyl-4-nitrophenol

To 3,5-dimethylphenol (9.00 g, 75 mmol) in 10 mL of ether was add a few drops of nitric acid (5 mL of 75% diluted with 20 mL of water). The reaction was cooled on ice and the remainder of the nitric acid solution was added dropwise. After 2 h the mixture was diluted with ether, washed with water, dried over sodium sulfate and the solvent evaporated. Chromatography on silica gel with hexane/ethyl acetate gave 3.4 g (27% yield) of product.

### Step 2

### 3,5-dimethyl-4-nitrophenyl 2-propyn-1-yl ether

3-bromo-1-propyne (0.375 g, 3.15 mmol) was added dropwise to 3,5-dimethyl-4-nitrophenol (0.50 g, 3.0 mmol) and potassium carbonate (0.517 g, 3.75 mmol) in 20 mL of DMF and the reaction was stirred at RT overnight. The DMF was removed under reduced pressure and the residue was dissolved in ethyl acetate, wash with water and brine, dried over sodium sulfate and the solvent evaporated. Chromatography on silica gel with hexane/ethyl acetate gave 0.50 g (81% yield) of product.

### Step 3

### 2,6-dimethyl-4-(propyloxy)aniline

A mixture 3,5-dimethyl-4-nitrophenyl 2-propyn-1-yl ether (0.250 g, 1.22 mmol) and palladium (10% on carbon, 0.200 g) in ca. 12 mL of ethanol was stirred under 60 psi of hydrogen for 20 h. The mixture was flushed with nitrogen, filtered, and the solvent evaporated to give 0.185 g (85% yield) of product.

### Step 4

### 2-isocyanato-1,3-dimethyl-5-(propyloxy)benzene

Phosgene (20% solution in toluene, 1.208 g) was added to a mixture of 2,6-dimethyl-4-(propyloxy)aniline (0.175 g, 0.976 mmol) and PS-DIEA (Argonaut Technologies, 0.626 g, 3.9 mmol/g) in 10 mL of dichloromethane. After stirring at RT overnight the mixture was filtered and concentrated to give 0.202 g (99% yield) of the product.

### Step 5

### methyl(2S)-cyclohexyl[({3-[({[2,6-dimethyl-4-(propyloxy)phenyl]amino}carbonyl)amino]-2-naphthalenyl}carbonyl)amino]ethanoate

Methyl(2S)-[(3-amino-2-naphthoyl) amino](cyclohexyl)ethanoate hydrochloride (0.184 g, 0.488 mmol) in 7 mL of pyridine was treated 2-isocyanato-1,3-dimethyl-5-(propyloxy)benzene (0.200 g, 0.97 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.125 g (47% yield) of product.

### Step 6

### (2S)-cyclohexyl[({3-[({[2,6-dimethyl-4-(propyloxy)phenyl]amino}carbonyl)amino]-2-naphthalenyl}carbonyl)amino]ethanoic acid

Lithium hydroxide monohydrate (0.075 g, 1.79 mmol) was added to a solution of methyl(2S)-cyclohexyl[({3-[({[2,6-dimethyl-4-(propyloxy)phenyl]amino}carbonyl)amino]-2-naphthalenyl}carbonyl)amino]ethanoate (0.120 g, 0.22 mmol) in THF:MeOH:water-9:3:3 mL. The mixture was stirred at RT overnight. The reaction mixture was acidified with 1N aqueous HCl and extracted with ethyl acetate. The organic phase was dried over sodium sulfate and concentrated to dryness to give 0.105 g (90% yield) of desired product as a white solid. ES MS m/z 530 (M−H).

### Example 474

### 2-cyclohexyl-N-[(2-{[({2,6-dichloro-4-[(trifluoromethyl)oxy]phenyl}amino)carbonyl]amino}-4-fluorophenyl)carbonyl]-L-alanine

### Step 1

### methyl 2-cyclohexyl-N-[(4-fluoro-2-nitrophenyl)carbonyl]-L-alaninate

HATU (0.498 g, 1.31 mmol) was added to a solution of 4-fluoro-2-nitrobenzoic acid (0.202 g, 1.09 mmol), methyl 2-cyclohexyl-L-alaninate hydrochloride (0.290 g, 1.31 mmol) and diisopropylethylamine (0.211 g, 1.63 mmol) in 10 mL of DMF. The mixture was stirred at RT overnight. The DMF was removed under reduced pressure and the residue was diluted with ethyl acetate. The organic layer was washed with sodium hydrogensulfate, sodium bicarbonate and brine, dried over sodium sulfate, filtered, and the solvent evaporated. Chromatography on silica gel with hexane/ethyl acetate gave 0.390 g (100% yield) of product.

### Step 2

### methyl N-[(2-amino-4-fluorophenyl)carbonyl]-2-cyclohexyl-L-alaninate

A mixture of methyl 2-cyclohexyl-N-[(4-fluoro-2-nitrophenyl)carbonyl]-L-alaninate (0.385 g, 1.09 mmol) and palladium (10% on carbon, 0.225 g) in ca. 25 mL of ethanol was stirred under 60 psi of hydrogen for 2 h. The mixture was flushed with nitrogen, filtered, and the solvent evaporated to give 0.362 g of product.

### Step 3

### methyl 2-cyclohexyl-N-[(2-{[({2,6-dichloro-4-[(trifluoromethyl)oxy]phenyl}amino)carbonyl]amino}-4-fluorophenyl)carbonyl]-L-alaninate

Methyl N-[(2-amino-4-fluorophenyl)carbonyl]-2-cyclohexyl-L-alaninate (0.155 g, 0.481 mmol) in 8 mL of pyridine was treated 1,3-dichloro-2-isocyanato-5-[(trifluoromethyl)oxy]benzene (0.261 g, 0.961 mmol) at RT overnight. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.158 g (55% yield) of product.

### Step 4

### 2-cyclohexyl-N-[(2-{[({2,6-dichloro-4-[(trifluoromethyl)oxy]phenyl}amino)carbonyl]amino}-4-fluorophenyl)carbonyl]-L-alanine

Lithium hydroxide monohydrate (0.100 g, 2.38 mmol) was added to a solution of methyl 2-cyclohexyl-N-[(2-{[({2,6-dichloro-4-[(trifluoromethyl)oxy]phenyl}amino)carbonyl]amino}-4-fluorophenyl)carbonyl]-L-alaninate (0.155 g, 0.26 mmol) in THF:MeOH:water-12:4:4 mL. The mixture was stirred at 60 C overnight. The reaction mixture was acidified with 1N aqueous HCl and extracted with ethyl acetate. The organic phase was dried over sodium sulfate and concentrated to dryness to give 0.136 g (90% yield) of desired product as a white solid. ES MS m/z 578 (M−H).

### Example 475

### (2S)-cyclohexyl({[2-({[(4-ethyl-2,6-dimethylphenyl)amino]carbonyl}amino)-4-fluorophenyl]carbonyl}amino)ethanoic acid

### Step 1

### methyl(2S)-cyclohexyl{[(4-fluoro-2-nitrophenyl)carbonyl]amino}ethanoate

HATU (2.37 g, 6.22 mmol) was added to a solution of 4-fluoro-2-nitrobenzoic acid (1.00 g, 5.41 mmol), methyl(2S)-amino(cyclohexyl)ethanoate hydrochloride (0.1.292 g, 6.22 mmol) and diisopropylethylamine (1.05 g, 8.11 mmol) in 50 mL of DMF. The mixture was stirred at RT overnight. The DMF was removed under reduced pressure and the residue was diluted with ethyl acetate. The organic layer was washed with sodium hydrogensulfate, sodium bicarbonate and brine, dried over sodium sulfate, filtered, and the solvent evaporated. Chromatography on silica gel with hexane/ethyl acetate gave 1.39 g (76% yield) of product.

### Step 2

### methyl(2S)-{[(2-amino-4-fluorophenyl)carbonyl]amino}(cyclohexyl)ethanoate

A mixture of methyl(2S)-cyclohexyl{[(4-fluoro-2-nitrophenyl)carbonyl]amino}ethanoate (1.39 g, 4.11 mmol) and palladium (10% on carbon, 0.75 g) in ca. 60 mL of ethanol was stirred under 60 psi of hydrogen for 2 h. The mixture was flushed with nitrogen, filtered, and the solvent evaporated to give 1.19 g (94% yield) of product.

### Step 3

### methyl(2S)-({[2-({[(4-bromo-2,6-dimethylphenyl)amino]carbonyl}amino)-4-fluorophenyl]carbonyl}amino)(cyclohexyl)ethanoate

Methyl(2S)-{[(2-amino-4-fluorophenyl)carbonyl]amino}(cyclohexyl)ethanoate (0.600 g, 1.95 mmol) in 40 mL of pyridine was treated 5-bromo-2-isocyanato-1,3-dimethylbenzene (1.10 g, 4.86 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.958 g (92% yield) of product.

### Step 4

### methyl(2S)-cyclohexyl({[2-({[(4-ethenyl-2,6-dimethylphenyl)amino]carbonyl}amino)-4-fluorophenyl]carbonyl}amino)ethanoate

Tetrakis(triphenylphosphine)palladium(0) (0.025 g, 0.022 mmol)was added to methyl(2S)-({[2-({[(4-bromo-2,6-dimethylphenyl)amino]carbonyl}amino)-4-fluorophenyl]carbonyl}amino)(cyclohexyl)ethanoate (0.200 g, 0.374 mmol) and tributyl(ethenyl)stannane (0.130 g, 0.412 mmol) in 4 mL of acetonitrile. The mixture was heated at 150 C for 30 min in a microwave reactor. The solvent was removed under reduced pressure and chromatography on silica gel with hexane/ethyl acetate gave 0.083 g (46% yield) of product.

### Step 5

### methyl(2S)-cyclohexyl({[2-({[(4-ethyl-2,6-dimethylphenyl)amino]carbonyl}amino)-4-fluorophenyl]carbonyl}amino)ethanoate

### Methyl(2S)-cyclohexyl({[2-({[(4-ethenyl-2,6-dimethylphenyl)amino]carbonyl}amino)-4-fluorophenyl]carbonyl}amino)ethanoate

(0.080 g, 0.166 mmol) and palladium (10% on carbon, 0.050 g) in ca. 12 mL of ethyl acetate was stirred under 60 psi of hydrogen for 2 h. The mixture was flushed with nitrogen, filtered, and the solvent evaporated to give 0.075 g (94% yield) of product.

### Step 6

### (2S)-cyclohexyl({[2-({[(4-ethyl-2,6-dimethylphenyl)amino]carbonyl}amino)-4-fluorophenyl]carbonyl}amino)ethanoic acid

Lithium hydroxide monohydrate (0.060 g, 1.43 mmol) was added to a solution of methyl(2S)-cyclohexyl({[2-({[(4-ethyl-2,6-dimethylphenyl)amino]carbonyl}amino)-4-fluorophenyl]carbonyl}amino)ethanoate (0.075 g, 0.155 mmol) in THF:MeOH:water-9:3:3 mL. The mixture was stirred at RT overnight. The reaction mixture was acidified with 1N aqueous HCl and extracted with ethyl acetate. The organic phase was dried over sodium sulfate and concentrated to dryness. Chromatography on silica gel with hexane/ethyl acetate gave 0.045 g (62% yield) of product as a white solid. ES MS m/z 468 (M−H).

### Example 476

### (2S)-cyclohexyl[({2-[({[2,6-dimethyl-4-(2-propen-1-yl)phenyl]amino}carbonyl)amino]-4-fluorophenyl}carbonyl)amino]ethanoic acid

### Step 1

### methyl(2S)-cyclohexyl[({2-[({[2,6-dimethyl-4-(2-propen-1-yl)phenyl]amino}carbonyl)amino]-4-fluorophenyl}carbonyl)amino]ethanoate

Tetrakis(triphenylphosphine)palladium(0) (0.032 g, 0.028 mmol) was added to methyl(2S)-({[2-({[(4-bromo-2,6-dimethylphenyl)amino]carbonyl}amino)-4-fluorophenyl]carbonyl}amino)(cyclohexyl)ethanoate (0.250 g, 0.468 mmol) and tributyl(2-propen-1-yl)stannane (0.170 g, 0.515 mmol) in 4.5 mL of acetonitrile. The mixture was heated at 150 C for 30 min in a microwave reactor. The solvent was removed under reduced pressure and chromatography on silica gel with hexane/ethyl acetate gave 0.178 g (77% yield) of product.

### Step 2

### (2S)-cyclohexyl[({2-[({[2,6-dimethyl-4-(2-propen-1-yl)phenyl]amino}carbonyl)amino]-4-fluorophenyl}carbonyl)amino]ethanoic acid

Lithium hydroxide monohydrate (0.060 g, 1.42 mmol) was added to a solution of methyl(2S)-cyclohexyl[({2-[({[2,6-dimethyl-4-(2-propen-1-yl)phenyl]amino}carbonyl)amino]-4-fluorophenyl}carbonyl)amino]ethanoate (0.055 g, 0.11 mmol) in THF:MeOH:water-9:3:3 mL. The mixture was stirred at RT overnight. The reaction mixture was acidified with 1N aqueous HCl and extracted with ethyl acetate. The organic phase was dried over sodium sulfate and concentrated to dryness to give 0.053 g (99% yield) of desired product as a white solid. ES MS m/z 480 (M−H).

### Example 477

### (2S)-cyclohexyl({[2-({[(2,6-dimethyl-4-propylphenyl)amino]carbonyl}amino)-4-fluorophenyl]carbonyl}amino)ethanoic acid

### Step 1

### methyl(2S)-cyclohexyl({[2-({[(2,6-dimethyl-4-propylphenyl)amino]carbonyl}amino)-4-fluorophenyl]carbonyl}amino)ethanoate

Methyl(2S)-cyclohexyl[({2-[({[2,6-dimethyl-4-(2-propen-1-yl)phenyl]amino}carbonyl)amino]-4-fluorophenyl}carbonyl)amino]ethanoate (0.112 g, 0.226 mmol) and palladium (10% on carbon, 0.090 g) in ca. 15 mL of ethyl acetate was stirred under 60 psi of hydrogen for 2 h. The mixture was flushed with nitrogen, filtered, and the solvent evaporated to give 0.105 g (93% yield) of product.

### Step 2

### (2S)-cyclohexyl({[2-({[(2,6-dimethyl-4-propylphenyl)amino]carbonyl}amino)-4-fluorophenyl]carbonyl}amino)ethanoic acid

Lithium hydroxide monohydrate (0.075 g, 1.79 mmol) was added to a solution of methyl(2S)-cyclohexyl({[2-({[(2,6-dimethyl-4-propylphenyl)amino]carbonyl}amino)-4-fluorophenyl]carbonyl}amino)ethanoate (0.105 g, 0.211 mmol) in THF:MeOH:water-9:3:3 mL. The mixture was stirred at RT overnight. The reaction mixture was acidified with 1N aqueous HCl and extracted with ethyl acetate. The organic phase was dried over sodium sulfate and concentrated to dryness to give 0.099 g (97% yield) of desired product as a white solid. ES MS m/z 482 (M−H).

### Example 478

### (2S)-cyclohexyl({[2-({[(2,6-dimethyl-4-pentylphenyl)amino]carbonyl}amino)-4-fluorophenyl]carbonyl}amino)ethanoic acid

### Step 1

### methyl(2S)-cyclohexyl{[(2-{[({2,6-dimethyl-4-[(1E)-1-penten-1-yl]phenyl}amino)carbonyl]amino}-4-fluorophenyl)carbonyl]amino}ethanoate

A mixture of methyl(2S)-({[2-({[(4-bromo-2,6-dimethylphenyl)amino]carbonyl}amino)-4-fluorophenyl]carbonyl}amino)(cyclohexyl)ethanoate (0.100 g, 0.187 mmol), (1E)-1-penten-1-ylboronic acid (0.023 g, 0.206 mmol), cesium fluoride (0.085 g, 0.561 mmol) and trans-dichlorobis(tricyclohexylphosphine)palladium (II) (0.007 g, 0.009 mmol) in 3 mL of acetonitrile and 1 mL of water was heated at 150 C for 6 min in a microwave reactor. The reaction was diluted with ethyl acetate and washed with water and brine. The solvent was removed under reduced pressure and chromatography on silica gel with hexane/ethyl acetate gave 0.078 g (80% yield) of product.

### Step 2

### methyl(2S)-cyclohexyl({[2-({[(2,6-dimethyl-4-pentylphenyl)amino]carbonyl}amino)-4-fluorophenyl]carbonyl}amino)ethanoate

Methyl(2S)-cyclohexyl{[(2-{[({2,6-dimethyl-4-[(1E)-1-penten-1-yl]phenyl}amino)carbonyl]amino}-4-fluorophenyl)carbonyl]amino}ethanoate (0.078 g, 0.149 mmol) and palladium (10% on carbon, 0.050 g) in ca. 8 mL of ethyl acetate was stirred under 60 psi of hydrogen for 2 h. The mixture was flushed with nitrogen, filtered, and the solvent evaporated to give 0.069 g (88% yield) of product.

### Step 3

### (2S)-cyclohexyl({[2-({[(2,6-dimethyl-4-pentylphenyl)amino]carbonyl}amino)-4-fluorophenyl]carbonyl}amino)ethanoic acid

Lithium hydroxide monohydrate (0.060 g, 1.42 mmol) was added to a solution of methyl(2S)-cyclohexyl({[2-({[(2,6-dimethyl-4-pentylphenyl)amino]carbonyl}amino)-4-fluorophenyl]carbonyl}amino)ethanoate (0.067 g, 0.127 mmol) in THF:MeOH:water-9:3:3 mL. The mixture was stirred at RT for 7 h. The reaction mixture was acidified with 1N aqueous HCl and extracted with ethyl acetate. The organic phase was dried over sodium sulfate and concentrated to dryness. Chromatography on silica gel with hexane/ethyl acetate gave 0.047 g (72% yield) of product as a white solid. ES MS m/z 510 (M−H).

### Example 479

### 2-cyclohexyl-N-{[2-({[(2,6-dimethyl-4-propylphenyl)amino]carbonyl}amino)-4-fluorophenyl]carbonyl}-L-alanine

### Step 1

### methyl N-{[2-({[(4-bromo-2,6-dimethylphenyl)amino]carbonyl}amino)-4-fluorophenyl]carbonyl}-2-cyclohexyl-L-alaninate

Methyl N-[(2-amino-4-fluorophenyl)carbonyl]-2-cyclohexyl-L-alaninate (0.175 g, 0.543 mmol) in 10 mL of pyridine was treated 5-bromo-2-isocyanato-1,3-dimethylbenzene (0.370 g, 1.63 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.245 g (82% yield) of product.

### Step 2

### methyl 2-cyclohexyl-N-({2-[({[2,6-dimethyl-4-(2-propen-1-yl)phenyl]amino}carbonyl)amino]-4-fluorophenyl}carbonyl)-L-alaninate

Tetrakis(triphenylphosphine)palladium(0) (0.03 g, 0.026 mmol)was added to methyl N-{[2-({[(4-bromo-2,6-dimethylphenyl)amino]carbonyl}amino)-4-fluorophenyl]carbonyl}-2-cyclohexyl-L-alaninate (0.240 g, 0.438 mmol) and tributyl(2-propen-1-yl)stannane (0.166 g, 0.503 mmol) in 5 mL of acetonitrile. The mixture was heated at 150 C for 30 min in a microwave reactor. The solvent was removed under reduced pressure and chromatography on silica gel with hexane/ethyl acetate gave 0.169 g (76% yield) of product.

### Step 3

### methyl 2-cyclohexyl-N-{[2-({[(2,6-dimethyl-4-propylphenyl)amino]carbonyl}amino)-4-fluorophenyl]carbonyl}-L-alaninate

Methyl 2-cyclohexyl-N-({2-[({[2,6-dimethyl-4-(2-propen-1-yl)phenyl]amino}carbonyl)amino]-4-fluorophenyl}carbonyl)-L-alaninate (0.165 g, 0.324 mmol) and palladium (10% on carbon, 0.115 g) in ca. 15 mL of ethyl acetate was stirred under 60 psi of hydrogen for 2 h. The mixture was flushed with nitrogen, filtered, and the solvent evaporated to give 0.160 g (96% yield) of product.

### Step 4

### 2-cyclohexyl-N-{[2-({[(2,6-dimethyl-4-propylphenyl)amino]carbonyl}amino)-4-fluorophenyl]carbonyl}-L-alanine

Lithium hydroxide monohydrate (0.100 g, 2.38 mmol) was added to a solution of methyl 2-cyclohexyl-N-{[2-({[(2,6-dimethyl-4-propylphenyl)amino]carbonyl}amino)-4-fluorophenyl]carbonyl}-L-alaninate (0.160 g, 0.313 mmol) in THF:MeOH:water-12:4:4 mL. The mixture was stirred at RT overnight. The reaction mixture was acidified with 1N aqueous HCl and extracted with ethyl acetate. The organic phase was dried over sodium sulfate and concentrated to dryness. Chromatography on silica gel with hexane/ethyl acetate gave 0.121 g (75% yield) of product as a white solid. ES MS m/z 496 (M−H).

### Example 480

### (2S)-({[2-({[(4-butyl-2,6-dimethylphenyl)amino]carbonyl}amino)-4-fluorophenyl]carbonyl}amino)(cyclohexyl)ethanoic acid

### Step 1

### 2-[(1E)-1-buten-1-yl]-1,3,2-benzodioxaborole

Butyne (ca. 4 g) was condensed into a sealable pressure bottle cooled in a −78 C bath. To this was added catecholborane (1 M in THF, 65 mL). The bottle was capped and after warming to RT was heated at 75 C overnight (blast shield used). The reaction was cooled to −78 C and the cap was removed carefully and the solvent evaporated. Distillation under reduce pressure (ca. 1 torr) gave 7.0 g of the product as a clear liquid.

### Step 2

### methyl(2S)-{[(2-{[({4-[(1E)-1-buten-1-yl]-2,6-dimethylphenyl}amino)carbonyl]amino}-4-fluorophenyl)carbonyl]amino}(cyclohexyl)ethanoate

A mixture of methyl(2S)-({[2-({[(4-bromo-2,6-dimethylphenyl)amino]carbonyl}amino)-4-fluorophenyl]carbonyl}amino)(cyclohexyl)ethanoate (0.135 g, 0.253 mmol), 2-[(1E)-1-buten-1-yl]-1,3,2-benzodioxaborole (0.048 g, 0.278 mmol), cesium fluoride (0.115 g, 0.759 mmol) and trans-dichlorobis(tricyclohexylphosphine)palladium (II) (0.009 g, 0.012 mmol) in 3.5 mL of acetonitrile and 1.2 mL of water was heated at 150 C for 7 min in a microwave reactor. The reaction was diluted with ethyl acetate and washed with water and brine. The organic phase was dried over sodium sulfate, the solvent was removed under reduced pressure, and chromatography on silica gel with hexane/ethyl acetate gave 0.093 g (72% yield) of product.

### Step 3

### methyl(2S)-({[2-({[(4-butyl-2,6-dimethylphenyl)amino]carbonyl}amino)-4-fluorophenyl]carbonyl}amino)(cyclohexyl)ethanoate

Methyl(2S)-{[(2-{[({4-[(1E)-1-buten-1-yl]-2,6-dimethylphenyl}amino)carbonyl]amino}-4-fluorophenyl)carbonyl]amino}(cyclohexyl)ethanoate

(0.090 g, 0.177 mmol) and palladium (10% on carbon, 0.075 g) in ca. 10 mL of ethyl acetate was stirred under 60 psi of hydrogen for 2 h. The mixture was flushed with nitrogen, filtered, and the solvent evaporated to give 0.089 g (99% yield) of product.

### Step 4

### (2S)-({[2-({[(4-butyl-2,6-dimethylphenyl)amino]carbonyl}amino)-4-fluorophenyl]carbonyl}amino)(cyclohexyl)ethanoic acid

Lithium hydroxide monohydrate (0.075 g, 1.79 mmol) was added to a solution of methyl(2S)-({[2-({[(4-butyl-2,6-dimethylphenyl)amino]carbonyl}amino)-4-fluorophenyl]carbonyl}amino)(cyclohexyl)ethanoate (0.089 g, 0.174 mmol) in THF:MeOH:water-9:3:3 mL. The mixture was stirred at RT overnight. The reaction mixture was acidified with 1N aqueous HCl and extracted with ethyl acetate. The organic phase was dried over sodium sulfate and concentrated to dryness. Chromatography on silica gel with hexane/ethyl acetate gave 0.051 g (59% yield) of product as a white solid. ES MS m/z 496 (M−H).

### Example 481

### O-(1,1-dimethylethyl)-N-{[3-({[(2,6-dimethyl-4-propylphenyl)amino]carbonyl}amino)-3′,4′-difluoro-4-biphenylyl]carbonyl}-L-threonine

### Step 1

### methyl N-{[3-({[(4-bromo-2,6-dimethylphenyl)amino]carbonyl}amino)-3′,4′-difluoro-4-biphenylyl]carbonyl}-O-(1,1-dimethylethyl)-L-threoninate

Methyl N-[(3-amino-3′,4′-difluoro-4-biphenylyl)carbonyl]-O-(1,1-dimethylethyl)-L-threoninate (0.470 g, 1.12 mmol) in 30 mL of pyridine was treated 5-bromo-2-isocyanato-1,3-dimethylbenzene (0.633 g, 2.79 mmol) overnight at RT. The pyridine was removed at reduced pressure and the residue was diluted with ethyl acetate and filtered. The ethyl acetate phase was washed with aqueous NaHCO₃, dried over sodium sulfate, filtered, and the solvent evaporated. Chromatography on silica gel with hexane/ethyl acetate gave 0.721 g (99% yield) of product.

### Step 2

### methyl O-(1,1-dimethylethyl)-N-({3-[({[2,6-dimethyl-4-(2-propen-1-yl)phenyl]amino}carbonyl)amino]-3′,4′-difluoro-4-biphenylyl}carbonyl)-L-threoninate

Tetrakis(triphenylphosphine)palladium(0) (0.024 g, 0.021 mmol)was added to methyl N-{[3-({[(4-bromo-2,6-dimethylphenyl)amino]carbonyl}amino)-3′,4′-difluoro-4-biphenylyl]carbonyl}-O-(1,1-dimethylethyl)-L-threoninate (0.225 g, 0.348 mmol) and tributyl(2-propen-1-yl)stannane (0.132 g, 0.40 mmol) in 4.5 mL of acetonitrile. The mixture was heated at 150 C for 30 min in a microwave reactor. The solvent was removed under reduced pressure and chromatography on silica gel with hexane/ethyl acetate gave 0.157 g (74% yield) of product.

### Step 3

### methyl O-(1,1-dimethylethyl)-N-{[3-({[(2,6-dimethyl-4-propylphenyl)amino]carbonyl}amino)-3′,4′-difluoro-4-biphenylyl]carbonyl}-L-threoninate

Methyl O-(1,1-dimethylethyl)-N-({3-[({[2,6-dimethyl-4-(2-propen-1-yl)phenyl]amino}carbonyl)amino]-3′,4′-difluoro-4-biphenylyl}carbonyl)-L-threoninate (0.155 g, 0.254 mmol) and palladium (10% on carbon, 0.120 g) in ca. 10 mL of ethyl acetate was stirred under 60 psi of hydrogen for 3 h. The mixture was flushed with nitrogen, filtered, and the solvent evaporated to give 0.0142 g (91% yield) of product.

### Step 4

### O-(1,1-dimethylethyl)-N-{[3-({[(2,6-dimethyl-4-propylphenyl)amino]carbonyl}amino)-3′,4′-difluoro-4-biphenylyl]carbonyl}-L-threonine

Lithium hydroxide monohydrate (0.100 g, 2.38 mmol) was added to a solution of methyl O-(1,1-dimethylethyl)-N-{[3-({[(2,6-dimethyl-4-propylphenyl)amino]carbonyl}amino)-3′,4′-difluoro-4-biphenylyl]carbonyl}-L-threoninate (0.140 g, 0.230 mmol) in THF:MeOH:water-9:3:3 mL. The mixture was stirred at RT overnight. The reaction mixture was acidified with 1N aqueous HCl and extracted with ethyl acetate. The organic phase was dried over sodium sulfate and concentrated to dryness. Chromatography on silica gel with hexane/ethyl acetate gave 0.023 g (17% yield) of product as a white solid. ES MS m/z 594 (M−H).

### Example 482

### (2S)-cyclohexyl[({2-[({[4-(cyclopropylmethyl)-2,6-dimethylphenyl]amino}carbonyl)amino]-4-fluorophenyl}carbonyl)amino]ethanoic acid

### Step 1

### methyl(2S)-({[2-({[(4-bromo-2,6-dimethylphenyl)amino]carbonyl}amino)-4-fluorophenyl]carbonyl}amino)(cyclohexyl)ethanoate

Methyl(2S)-{[(2-amino-4-fluorophenyl)carbonyl]amino}(cyclohexyl)ethanoate (0.300 g, 0.97 mmol) in 20 mL of pyridine was treated 5-bromo-2-isocyanato-1,3-dimethylbenzene (0.552 g, 2.435 mmol) overnight at RT. The pyridine was removed at reduced pressure and the residue was partitioned between ethyl acetate and aqueous NaHCO₃. The organic layer was washed with brine, dried over sodium sulfate, filtered, and the solvent evaporated. Chromatography on silica gel with hexane/ethyl acetate gave 0.481 g (93% yield) of product.

### Step 2

### methyl(2S)-cyclohexyl[({2-[({[2,6-dimethyl-4-(2-propen-1-yl)phenyl]amino}carbonyl)amino]-4-fluorophenyl}carbonyl)amino]ethanoate

Tetrakis(triphenylphosphine)palladium(0) (0.062 g, 0.054 mmol)was added to methyl(2S)-({[2-({[(4-bromo-2,6-dimethylphenyl)amino]carbonyl}amino)-4-fluorophenyl]carbonyl}amino)(cyclohexyl)ethanoate (0.480 g, 0.898 mmol) and tributyl(2-propen-1-yl)stannane (0.327 g, 0.988 mmol) in 5 mL of acetonitrile. The mixture was heated at 150 C for 30 min in a microwave reactor. The solvent was removed under reduced pressure and chromatography on silica gel with hexane/ethyl acetate gave 0.309 g (69% yield) of product.

### Step 3

### methyl(2S)-cyclohexyl[({2-[({[4-(cyclopropylmethyl)-2,6-dimethylphenyl]amino}carbonyl)amino]-4-fluorophenyl}carbonyl)amino]ethanoate

Diazomethane (1.2 mmol in 10 mL dichloromethane, generated from N-methyl-N-nitrosourea) was added drop-wise to an ice cold solution of methyl(2S)-cyclohexyl[({2-[({[2,6-dimethyl-4-(2-propen-1-yl)phenyl]amino}carbonyl)amino]-4-fluorophenyl}carbonyl)amino]ethanoate (0.100 g, 0.202 mmol) and palladium (II) acetylacetonate (0.003 g, 0.01 mmol) in 10 mL of ether over ca. 20 min. The reaction was stirred on ice for 15 min, then warmed to RT over 30 min. The mixture was flushed with nitrogen, filtered through celite and concentrated to dryness. NMR showed this to be a mixture of product and starting material, so the material was subjected to the reaction procedure again. The resulting material was chromatographed on silica gel with hexane/ethyl acetate to give 0.080 g (78% yield) of the product.

### Step 4

### (2S)-cyclohexyl[({2-[({[4-(cyclopropylmethyl)-2,6-dimethylphenyl]amino}carbonyl)amino]-4-fluorophenyl}carbonyl)amino]ethanoic acid

Lithium hydroxide monohydrate (0.075 g, 1.79 mmol) was added to a solution of methyl(2S)-cyclohexyl[({2-[({[4-(cyclopropylmethyl)-2,6-dimethylphenyl]amino}carbonyl)amino]-4-fluorophenyl}carbonyl)amino]ethanoate (0.080 g, 0.157 mmol) in THF:MeOH:water-9:3:3 mL. The mixture was stirred at RT overnight. The reaction mixture was acidified with 1N aqueous HCl and extracted with ethyl acetate. The organic phase was dried over sodium sulfate and concentrated to dryness to give 80 mg (100% yield) of desired product as a white solid. ES MS m/z 494 (M−H).

### Example 483

### N-({3-[({[4-(cyclopropylmethyl)-2,6-dimethylphenyl]amino}carbonyl)amino]-3′,4′-difluoro-4-biphenyly}carbonyl)-0-(1,1-dimethylethyl)-L-threonine

### Step 1

### methyl O-(1,1-dimethylethyl)-N-({3-[({[2,6-dimethyl-4-(2-propen-1-yl)phenyl]amino}carbonyl)amino]-3′,4′-difluoro-4-biphenylyl}carbonyl)-L-threoninate

Tetrakis(triphenylphosphine)palladium(0) (0.024 g, 0.021 mmol)was added to methyl N-{[3-({[(4-bromo-2,6-dimethylphenyl)amino]carbonyl}amino)-3′,4′-difluoro-4-biphenylyl]carbonyl}-O-(1,1-dimethylethyl)-L-threoninate (0.225 g, 0.348 mmol) and tributyl(2-propen-1-yl)stannane (0.132 g, 0.40 mmol) in 4.5 mL of acetonitrile. The mixture was heated at 150 C for 20 min in a microwave reactor. The solvent was removed under reduced pressure and chromatography on silica gel with hexane/ethyl acetate gave 0.165 g (78% yield) of product.

### Step 2

### methyl N-({3-[({[4-(cyclopropylmethyl)-2,6-dimethylphenyl]amino}carbonyl)amino]-3′,4′-difluoro-4-biphenylyl}carbonyl)-O-(1,1-dimethylethyl)-L-threoninate

Diazomethane (2.42 mmol in 20 mL dichloromethane, generated from N-methyl-N-nitrosourea) was added drop-wise to an ice cold solution of methyl O-(1,1-dimethylethyl)-N-({3-[({[2,6-dimethyl-4-(2-propen-1-yl)phenyl]amino}carbonyl)amino]-3′,4′-difluoro-4-biphenylyl}carbonyl)-L-threoninate (0.125 g, 0.206 mmol) and palladium (II) acetylacetonate (0.003 g, 0.01 mmol) in 12 mL of ether over ca. 30 min. The reaction was stirred on ice for 10 min, then warmed to RT over 30 min. The mixture was flushed with nitrogen, filtered through celite and concentrated to dryness. Chromatography on silica gel with hexane/ethyl acetate to give 0.103 g (80% yield) of the product.

### Step 3

### N-({3-[({[4-(cyclopropylmethyl)-2,6-dimethylphenyl]amino}carbonyl)amino]-3′,4′-difluoro-4-biphenylyl}carbonyl)-O-(1,1-dimethylethyl)-L-threonine

Lithium hydroxide monohydrate (0.075 g, 1.79 mmol) was added to a solution of methyl N-({3-[({[4-(cyclopropylmethyl)-2,6-dimethylphenyl]amino}carbonyl)amino]-3′,4′-difluoro-4-biphenylyl}carbonyl)-O-(1,1-dimethylethyl)-L-threoninate (0.100 g, 0.161 mmol) in THF:MeOH:water-9:3:3 mL. The mixture was stirred at RT for 4 h. The reaction mixture was acidified with 1N aqueous HCl and extracted with ethyl acetate. The organic phase was dried over sodium sulfate and concentrated to dryness to give 0.091 g (93% yield) of desired product as a white solid. ES MS m/z 606 (M−H).

### Example 484

### (2S)-Cyclohexyl({[2-[4-(methyloxy)phenyl]-4-({[(2,4,6-trichlorophenyl)amino]carbonyl}amino)-1,3-thiazol-5-yl]carbonyl}amino)ethanoic acid

### Step 1

### Methyl 4-methoxybenzenecarbodithioate

Methyl 4-methoxybenzoate (10.15 g, 61.08 mmol) and Davy Reagent (17.37 g, 61.08 mmol) were combined in 1,2,4-trichlorobenzene. The reaction was heated to 200° C. in an oil bath and stirred for 1 h. Cooled to rt and poured onto a column of silica and eluted with hex/Ether (15/1). The solvent was stripped off and the residue was placed under vacuum until constant weight was obtained to give 12.08 g (61 mmol, 100%) of product as a red oil.

### Step 2

### Methyl 4-amino-2-(4-methoxyphenyl)-1,3-thiazole-5-carboxylate

Methyl 4-methoxybenzenecarbodithioate (5.43 g, 26.88 mmol), cyanamide (1.13 g, 26.88 mmol) and potassium methoxide (1.885 g, 26.88 mmol) were combined in dry methanol(100 mL) and stirred at rt for 4 h. The reaction was concentrated down to give a red solid. The residue was dissolved in dry DMF (100 mL) and Mel (5.723 g, 40.32 mmol) was added. The reaction went from dark red to light clear red immediately upon addition. Stirred for 2.5 h at RT and then diluted with EtOAc (300 mL), washed with water (3×500 mL) dried over MgSO₄, filtered and concentrated. The residue was taken up in dry methanol (100 mL) and then ethyl thioglycolate (26.88 mmol, 3.23 g) and triethylamine (80.64 mmol, 8.15 g) were added. The reaction was stirred for 1 h and precipitate was collected. Mother liquor was allowed to stir over night and a small second crop was collected. Total of 1.7 g (6.11 mmol, 23%) of the desired product, as a light yellow solid was collected.

### Step 3

### 4-Amino-2-[4-(methyloxy)phenyl]-1,3-thiazole-5-carboxylic acid

Methyl 4-amino-2-(4-methoxyphenyl)-1,3-thiazole-5-carboxylate (1.1 g, 3.96 mmol) was taken up in dioxane (20 mL). 1 M lithium hydroxide (20 mL) was added and the reaction stirred overnight at 80° C. The reaction was cooled to RT and neutralized with 1N HCl. Diluted with water (50 mL). Product was water soluble, so a large excess of sodium chloride was added. Precipitate was collected to give 1.1 g (4.4 mmol, 111%) of product as a light yellow solid.

### Step 4

### Methyl(2S)-[({4-amino-2-[4-(methyloxy)phenyl]-1,3-thiazol-5-yl}carbonyl)amino](cyclohexyl)ethanoate

(4-Amino-2-[4-(methyloxy)phenyl]-1,3-thiazole-5-carboxylic acid (0.56 g, 0.24 mmol) and methyl(2S)-amino(cyclohexyl)ethanoate (0.556 g, 2.69 mmol) were combined in DMF (10 mL). Triethylamine (0.67 mL, 4.93 mmol) was added followed by HATU (1.28 g, 3.36 mmol). The reaction was stirred for 2 d. The reaction was then diluted with EtOAc (50 mL) and washed with water (2×50 mL). The organics were dried over MgSO₄, filtered, concentrated and purified on a chromatatron (1:1 Hex:EtOAc) to afford 0.456 g (1.13 mmol, 51%) of the product as a yellow solid.

### Step 5

### (2S)-cyclohexyl({[2-[4-(methyloxy)phenyl]-4-({[(2,4,6-trichlorophenyl)amino]carbonyl}amino)-1,3-thiazol-5-yl]carbonyl}amino)ethanoic acid

Methyl(2S)-[({4-amino-2-[4-(methyloxy)phenyl]-1,3-thiazol-5-yl}carbonyl)amino](cyclohexyl)ethanoate (0.05 g, 0.12 mmol) was suspended in toluene (1 mL) and heated to 120° C. 1,3,5-Trichloro-2-isocyanatobenzene (0.03 g, 0.14 mmol) was added. The reaction was stirred overnight at 120° C. The reaction was concentrated to dryness and the residue purified on a chromatatron (3:1 Hex:EtOAc). The desired product and the starting thiazole coeluted. The mixture was dissolved in THF (1 mL) and 1 M lithium hydroxide was added and the reaction stirred overnight. Concentrated and purified on a Gilson to separate. Lyophilized to afford 0.009 g,(12%) of the product. ES MS m/z 611 (M+H).

### Example 485

### 1-({[2-[4-(methyloxy)phenyl]-5-({[(2,4,6-trimethylphenyl)amino]carbonyl}amino)-1,3-thiazol-4-yl]carbonyl}amino)cyclohexanecarboxylic acid

### Step 1

### Methyl 1-({[2-[4-(methyloxy)phenyl]-5-({[(2,4,6-trimethylphenyl)amino]carbonyl}amino)-1,3-thiazol-4-yl]carbonyl}amino)cyclohexanecarboxylate

Methyl(2S)-[({4-amino-2-[4-(methyloxy)phenyl]-1,3-thiazol-5-yl}carbonyl)amino](cyclohexyl)ethanoate (0.099 g, 0.25 mmol) was suspended in toluene (1 mL) and heated to 120° C. 2-Isocyanato-1,3,5-trimethylbenzene (0.119 g, 0.74 mmol) was added. The reaction was stirred overnight during which the reaction went dry. The residue was taken up in minimal methylene chloride and purified on a chromatatron (1:1 Hex:EtOAc) to give 0.06 g of an impure material. The impure material was repurified on a chromatatron (100% CH₂Cl₂) to give 0.04 g (0.071 mmol, 29%) of the desired product.

### Step 2

### 1-({[2-[4-(methyloxy)phenyl]-5-({[(2,4,6-trimethylphenyl)amino]carbonyl}amino)-1,3-thiazol-4-yl]carbonyl}amino)cyclohexanecarboxylic acid

Methyl(2S)-cyclohexyl({[2-[4-(methyloxy)phenyl]-4-({[(2,4,6-trimethylphenyl)amino]carbonyl}amino)-1,3-thiazol-5-yl]carbonyl}amino)ethanoate (0.04 g, 0.07 mmol) was taken up in dioxane (1 mL) and treated with 1 M lithium hydroxide (1 mL). The reaction was heated to 100° C. and monitored by LCMS, until all of the start material was gone. The reaction was cooled to RT and acidified with 1N HCl. The reaction was diluted with water (20 mL), and extracted with EtOAc (2×40 mL). The organics were dried over MgSO₄, filtered and concentrated to give 0.028 g (0.05 mol, 72%) of the product as a light brown gum. ES MS m/z 551 (M+H).

### Example 486

### (2S)-cyclohexyl({[2-[4-(methyloxy)phenyl]-5-({[(2,4,6-trimethylphenyl)amino]carbonyl}amino)-1,3-thiazol-4-yl]carbonyl}amino)ethanoic acid

### Step 1

### Methyl(2S)-cyclohexyl({[2-[4-(methyloxy)phenyl]-5-({[(2,4,6-trimethylphenyl)amino]carbonyl}amino)-1,3-thiazol-4-yl]carbonyl}amino)ethanoate

Methyl(2S)-[({5-amino-2-[4-(methyloxy)phenyl]-1,3-thiazol-4-yl}carbonyl)amino](cyclohexyl)ethanoate (0.069 g, 0.17 mmol) was suspended in toluene (1 mL) and heated to 120° C. 2-Isocyanato-1-methyl-3-(1-methylethyl)benzene (0.09 g, 0.51 mmol) was added. The reaction was stirred overnight during which the reaction went dry. The residue was taken up in minimal methylene chloride and purified on a chromatatron (1:1 Hex:EtOAc) to give 0.06 g of an impure material. The impure material was re-purified on a chromatatron (100% CH₂Cl₂) to give 0.019 g (0.0032 mmol, 19%) of the desired product.

### Step 2

### (2S)-cyclohexyl({[2-[4-(methyloxy)phenyl]-5-({[(2,4,6-trimethylphenyl)amino]carbonyl}amino)-1,3-thiazol-4-yl]carbonyl}amino)ethanoic acid

Methyl(2S)-cyclohexyl({[2-[4-(methyloxy)phenyl]-5-({[(2,4,6-trimethylphenyl)amino]carbonyl}amino)-1,3-thiazol-4-yl]carbonyl}amino)ethanoate (0.019 g, 0.03 mmol) was taken up in dioxane (1 mL) and treated with 1M lithium hydroxide (1 mL). The reaction was heated to 100° C. and monitored by LCMS, until all of the start material was gone. The reaction was cooled to rt and acidified with 1N HCl. Diluted with water (20 mL), and extracted with EtOAc (2×40 mL). Organics were dried over MgSO₄, filtered and concentrated to give 0.014 g (0.024 mmol, 76%) of the desired product as a light brown gum. ES MS m/z 565 (M+H).

### Example 487

### (2S)-Cyclohexyl[({5-({[(2,6-dichlorophenyl)amino]carbonyl}amino)-2-[4-(methyloxy)phenyl]-1,3-thiazol-4-yl}carbonyl)amino]ethanoic acid

### Step 1

### Methyl(2S)-cyclohexyl[({5-({[(2,6-dichlorophenyl)amino]carbonyl}amino)-2-[4-(methyloxy)phenyl]-1,3-thiazol-4-yl}carbonyl)amino]ethanoate

Methyl(2S)-[({5-amino-2-[4-(methyloxy)phenyl]-1,3-thiazol-4-yl}carbonyl)amino](cyclohexyl)ethanoate (0.089 g, 0.22 mmol) was suspended in toluene (1 mL) and heated to 120° C. 1,3-Dichloro-2-isocyanatobenzene (0.124 g, 0.66 mmol) was added. The reaction was stirred overnight during which the reaction went dry. The residue was taken up in minimal methylene chloride and purified on a chromatatron (3:1 Hex:EtOAc) to give 0.022 g (0.037 mmol, 17%) of the desired product.

### Step 2

### (2S)-Cyclohexyl[({5-({[(2,6-dichlorophenyl)amino]carbonyl}amino)-2-[4-(methyloxy)phenyl]-1,3-thiazol-4-yl}carbonyl)amino]ethanoic acid

Methyl(2S)-[({5-amino-2-[4-(methyloxy)phenyl]-1,3-thiazol-4-yl}carbonyl)amino](cyclohexyl)ethanoate (0.022 g, 0.04 mmol) was taken up in dioxane (1 mL) and treated with 1 M lithium hydroxide (1 mL). The reaction was heated to 100° C. and monitored by LCMS, until all of the start material was gone. The reaction was cooled to rt and acidified with 1N HCl. Diluted with water (20 mL), and extracted with EtOAc (2×40 mL). The organics were dried over MgSO₄, filtered and concentrated to give 0.019 g (0.032 mmol, 88%) of the desired product as a light brown solid. ES MS m/z 577 (M+H), 599 (M+Na).

### Example 488

### 2-[4-(Methyloxy)phenyl]-5-({[(2,4,6-trimethylphenyl)amino]carbonyl}amino)-1,3-thiazole-4-carboxylic acid

### Step 1

### Diethyl({[⁴-(methyloxy)phenyl]carbonyl}amino)propanedioate

4-Diethyl aminopropanedioate (25.32 g, 148.42 mmol) and sodium bicarbonate (15.73 g, 148.42 mmol) were combined in a biphasic mixture of water (200 mL) and methylene chloride (200 mL). (Methyloxy)benzoyl chloride (27.248, 148.42 mmol) was added and the reaction stirred overnight at rt. The organics were removed and then washed with water (2×100 mL), dried over MgSO₄, filtered and concentrated to give 42.35 g (150 mmol, 100%) of the product as a white solid.

### Step 2

### Ethyl 5-(ethyloxy)-2-[4-(methyloxy)phenyl]-1,3-oxazole-4-carboxylate

Diethyl({[4-(methyloxy)phenyl]carbonyl}amino)propanedioate (21.62 g, 76.94 mmol) was dissolved in chloroform (200 mL). Phosphorous pentachloride (16.022 g, 76.94 mmol) was added and the reaction heated to reflux. The reaction was stirred for 2.5 days. The reaction was concentrated and the residue taken up in Ether (500 mL). The reaction was then poured on to ice and then neutralized with solid Sodium Bicarb. The organics were separated and dried over MgSO₄, filtered and concentrated. The residue was divided into several portions and purified on ISCO to give 10.5 g of pure product.

### Step 3

### 5-(ethyloxy)-2-[4-(methyloxy)phenyl]-1,3-oxazole-4-carboxylic acid

Ethyl 5-(ethyloxy)-2-[4-(methyloxy)phenyl]-1,3-oxazole-4-carboxylate (10.50 g, 36.08 mmol) was taken up in THF (100 mL). 1 M lithium hydroxide (40 mL) was added and heated to 70° C. The reaction was stirred for 2 h until complete by TLC. The reaction was cooled and acidified with 1N HCl, white precipitate was formed. The precipitate was collected and dried to give 7.97 g (30.30 mmol, 84%) of the product as a white solid.

### Step 4

### 5-(Ethyloxy)-2-[4-(methyloxy)phenyl]-1,3-oxazole-4-carboxamide

5-[(Ethyloxy)carbonyl]-2-[4-(methyloxy)phenyl]-1,3-oxazole-4-carboxylic acid (1.78 g, 6.77 mmol) was suspended in methylene chloride (100 mL) and DMF (0.02 mL) was added. Oxalyl Chloride (2.64 mL, 6.77 mmol) was added dropwise and the reaction stirred overnight at rt. The reaction was concentrated and the excess oxalyl chloride was azetroped off with methylene chloride to give an off white solid. Aqueous ammonia (50 mL) was cooled in an ice bath. The acid chloride was suspended in minimal THF and then added slowly to the ammonia. The reaction was stirred for 6 h. The precipitate was collected to give 1.394 g (5.32 mmol, 79%) of the desired product as an off white solid.

### Step 5

### Ethyl 5-amino-2-[4-(methyloxy)phenyl]-1,3-thiazole-4-carboxylate

5-(Ethyloxy)-2-[4-(methyloxy)phenyl]-1,3-oxazole-4-carboxamide (4.37 g, 16.68 mmol) and Lawesson's Reagent (13.492 g, 33.36 mmol) were taken up in THF (100 mL) and heated to 70° C. and stirred overnight. The reaction was cooled to rt and filtered through celite. The reaction was concentrated and then purified on an ISCO (20% EtOAc in Hex) to give 1.6 g (5.75 mmol, 35%) of the product as a pink solid.

### Step 6

### 5-Amino-2-[4-(methyloxy)phenyl]-1,3-thiazole-4-carboxylic acid

Ethyl 5-amino-2-[4-(methyloxy)phenyl]-1,3-thiazole-4-carboxylate (1.08 g, 3.89 mmol) was taken up in THF (100 mL) and 1 M Lithium hydroxide (10 mL) was added. The reaction was heated to 70° C. and was stirred for 3d, until all start material was gone. The reaction was cooled and acidified with 1N HCl. The reaction was then extracted with EtOAc (2×200 mL). The combined organics were dried over MgSO₄, filtered and concentrated to give 1 g (4 mmol, 100%) of the product as an orange solid.

### Step 7

### 2-[4-(Methyloxy)phenyl]-5-({[(2,4,6-trimethylphenyl)amino]carbonyl}amino)-1,3-thiazole-4-carboxylic acid

4-Amino-2-[4-(methyloxy)phenyl]-1,3-thiazole-5-carboxylic acid (0.105 g, 0.42 mmol) and 2-Isocyanato-1,3,5-trimethylbenzene (0.203 g, 1.26 mmol) were combined in toluene and heated to 120° C. The reaction was stirred overnight and reaction went dry. The reaction was cooled and the residue was purified on a chromatatron (2.5% methanol in methylene chloride) to afford 0.028 g (0.07 mmol, 16%) of the product as a dull yellow solid.

### Example 489

### 1-({[2-[4-(Methyloxy)phenyl]-5-({[(2,4,6-trimethylphenyl)amino]carbonyl}amino)-1,3-thiazol-4-yl]carbonyl}amino)cyclohexanecarboxylic acid

### Step 1

### Methyl 1-[({5-amino-2-[4-(methyloxy)phenyl]-1,3-thiazol-4-yl}carbonyl)amino]cyclohexanecarboxylate

5-Amino-2-[4-(methyloxy)phenyl]-1,3-thiazole-4-carboxylic acid (0.088 g, 0.35 mmol) and methyl 1-aminocyclohexanecarboxylate (0.068 g, 0.35 mmol) were combined in DMF (5 mL). Triethylamine (0.105 mL, 0.77 mmol) and HATU (0.201 g, 0.53 mmol) were added. The reaction was stirred over night at rt. The reaction was partioned between water (50 mL) and EtOAc (50 mL). The aqueous fraction was removed and the organics were washed with water (2×50 mL), brine (1×50 mL), dried over MgSO₄, filtered and concentrated. The residue purified on an ISCO (1:1 Hex:EtOAc) to give 0.050 g (0.128 mmol, 37%).

### Step 2

### Methyl 1-({[2-[4-(methyloxy)phenyl]-5-({[(2,4,6-trimethylphenyl)amino]carbonyl}amino)-1,3-thiazol-4-yl]carbonyl}amino)cyclohexanecarboxylate

Methyl 1-[({5-amino-2-[4-(methyloxy)phenyl]-1,3-thiazol-4-yl}carbonyl)amino]cyclohexanecarboxylate (0.046 g, 0.12 mmol) was taken up in toluene and heated to 120° C. 2-Isocyanato-1,3,5-trimethylbenzene (0.038 g, 0.24 mmol) was add and the reaction stirred for 2 h. Additional isocyanate (0.057 g, 0.36 mmol) was added and the reaction stirred overnight. The reaction was concentrated and the residue purified on a chromatatron (100% CH₂Cl₂) to give 0.040 g (0.07 mmol, 62%) of the product as a light brown semisolid.

### Step 3

### 1-({[2-[4-(Methyloxy)phenyl]-5-({[(2,4,6-trimethylphenyl)amino]carbonyl}amino)-1,3-thiazol-4-yl]carbonyl}amino)cyclohexanecarboxylic acid

Methyl 1-({[2-[4-(methyloxy)phenyl]-5-({[(2,4,6-trimethylphenyl)amino]carbonyl}amino)-1,3-thiazol-4-yl]carbonyl}amino)cyclohexanecarboxylate (0.040, 0.08 mmol) was taken up in dioxane (1 mL) and 1 M Lithium hydroxide (1 mL) was added. The reaction was heated to 100° C. and stirred for 1 h. The reaction was cooled and neutralized with 1N HCl. The reaction was diluted with water (10 mL) and extracted with EtOAc (2×20 mL). The organics were washed with water (2×20 mL), brine (1×20 mL), dried over MgSO₄, filtered and concentrated. The residue was placed under vacuum, until a constant weight was obtained, to give 0.021 g (0.039 mmol, 47%) of the product as a light tan solid. ES MS m/z 537 (M+H).

### Example 490

### (2S)-({[2-(4-Chlorophenyl)-5-({[(2,4,6-trichlorophenyl)amino]carbonyl}amino)-1,3-thiazol-4-yl]carbonyl}amino)(cyclohexyl)ethanoic acid

### Step 1

### Diethyl {[(4-chlorophenyl)carbonyl]amino}propanedioate

4-Diethyl aminopropanedioate (20.72 g, 97.90 mmol) and sodium bicarbonate (10.37 g, 97.90 mmol) were combined in a biphasic mixture of water (200 mL) and methylene chloride (200 mL). 4-chlorobenzoyl chloride (17.13, 97.90 mmol) was added and the reaction stirred overnight at rt. The organics were removed and then washed with water (2×100 mL), dried over MgSO₄, filtered and concentrated to give 29.38 g (195.08 mmol 97%) of the product as a white solid.

### Step 2

### Ethyl 2-(4-chlorophenyl)-5-(ethyloxy)-1,3-oxazole-4-carboxylate

Diethyl {[(4-chlorophenyl)carbonyl]amino}propanedioate (29.38 g, 104.56 mmol) was dissolved in chloroform (200 mL). Phosphorous pentachloride (21.77 g, 104.56 mmol) was added and the reaction heated to reflux. The reaction was stirred for 4 days. The reaction was concentrated and the residue taken up in Ether (500 mL). The organics were poured on to ice and then neutralized with solid Sodium Bicarb. The organics were separated and dried over MgSO₄, filtered and concentrated to give 28.4 g (107 mmol, 100%) of product as a white solid.

### Step 3

### 2-(4-Chlorophenyl)-5-(ethyloxy)-1,3-oxazole-4-carboxylic acid

Ethyl 2-(4-chlorophenyl)-5-(ethyloxy)-1,3-oxazole-4-carboxylate (28.4 g, 96.27 mmol) was taken up in THF (200 mL). 1 M Lithium hydroxide (100 mL) was added and heated to 70° C. The reaction was stirred overnight, then cooled and neutralized with 1N HCl. A white precipitate was formed. The reaction was extracted with EtOAc (2×200 mL) and the combined organics were washed with water (1×200 mL), brine (1×200 mL), dried over MgSO₄, filtered and concentrated to give 2.56 g (8.67 mmol, 9%) of starting ester. The aqueous fraction was acidified with 1N HCl and reextracted with EtOAc (2×300 mL). The organics were washed with water (1×200 mL), brine (1×200 mL), dried over MgSO₄, filtered and concentrated to give 18.56 g (69.51 mmol, 72%) of the desired product as a fluffy white solid.

### Step 4

### 2-(4-Chlorophenyl)-5-(ethyloxy)-1,3-oxazole-4-carboxamide

2-(4-Chlorophenyl)-5-(ethyloxy)-1,3-oxazole-4-carboxylic acid (4.27 g, 15.99 mmol) was suspended in methylene chloride (50 mL) and DMF (0.02 mL) was added. Oxalyl Chloride (1.54 mL, 17.59 mmol) was added dropwise and reaction warmed to 50° C. and stirred overnight. The reaction was concentrated and the residue taken up in dioxane (50 mL). Ammonia in dioxane (68 mL of a 0.5M sol) was added via an addition funnel. The reaction was stirred at rt for 3 h. The reaction was concentrated and the residue taken up in minimal methylene chloride. The organics were triturated with ether and the precipitate removed. The mother liquor was concentrated to give 4.6 g (17.36 mmol, 108%) of product as a tan solid.

### Step 5

### 2-(4-Chlorophenyl)-5-(ethyloxy)-1,3-oxazole-4-carbothioamide

2-(4-Chlorophenyl)-5-(ethyloxy)-1,3-oxazole-4-carboxamide 2.83 g, 10.68 mmol) and Lawesson's Reagent were combined in THF (100 mL), heated to 70° C. and stirred for 3 h. The reaction was concentrated and charged to a flash column. The product was eluted with 1:1 Hex:EtOAc to obtain contaminated product. The crude material was triturated with hexane to afford 0.176 g (0.62 mmol, 6%) of the product.

### Step 6

### ethyl 5-amino-2-(4-chlorophenyl)-1,3-thiazole-4-carboxylate

### 2-(4-Chlorophenyl)-5-(ethyloxy)-1,3-oxazole-4-carbothioamide (0.190 g, 0.68 mmol) was taken up in toluene (5 mL), heated to 110° C. and stirred overnight. The reaction was concentrated to afford 0.19 g (0.68 mmol, 100%) of the product as a light tan solid.

### Step 7

### Methyl(2S)-({[5-amino-2-(4-chlorophenyl)-1,3-thiazol-4-yl]carbonyl}amino)(cyclohexyl)ethanoate

5-Amino-2-(4-chlorophenyl)-1,3-thiazole-4-carboxylic acid (0.149 g, 0.59 mmol) and methyl(2S)-amino(cyclohexyl)ethanoate (0.121 g, 0.59 mmol) were combined in DMF (50 mL). Triethylamine (0.13 g, 1.29 mmol) was added followed by HATU (0.334 g, 0.88 mmol). The reaction was stirred overnight at rt. The reaction was partioned between EtOAc (50 mL) and with water (50 mL). The organics were washed with water (2×50 mL), brine (1×50 mL), dried over MgSO₄, filtered and concentrated. The crude material was purified on a chromatatron (5:1 Hex:EtOAc) to afford 0.06 g (0.147 mmol, 25%) of the product as a yellow solid.

### Step 8

### Methyl(2S)-({[2-(4-chlorophenyl)-5-({[(2,4,6-trichlorophenyl)amino]carbonyl}amino)-1,3-thiazol-4-yl]carbonyl}amino)(cyclohexyl)ethanoate

Methyl(2S)-({[5-amino-2-(4-chlorophenyl)-1,3-thiazol-4-yl]carbonyl}amino)(cyclohexyl)ethanoate (0.056 g, 0.14 mmol) and 2-isocyanato-1,3,5-trimethylbenzene (0.092 g, 0.41 mmol) were combined in DMF and heated to 120° C. The reaction was stirred for 3 h and then concentrated. The residue was purified on a chromatatron (8:1 Hex:EtOAc) to afford a binary mixture. The crude material was repurified on a chromatatron (100% CH₂Cl₂) to give 0.02 g (0.032 mmol, 23%) of the desired product.

### Step 9

### (2S)-({[2-(4-Chlorophenyl)-5-({[(2,4,6-trichlorophenyl)amino]carbonyl}amino)-1,3-thiazol-4-yl]carbonyl}amino)(cyclohexyl)ethanoic acid

Methyl(2S)-({[2-(4-chlorophenyl)-5-({[(2,4,6-trimethylphenyl)amino]carbonyl}amino)-1,3-thiazol-4-yl]carbonyl}amino)(cyclohexyl)ethanoate (0.02 g, 0.03 mmol) was taken up in THF (1 mL) and 1 M lithium hydroxide (0.03 mL) was added. The reaction was heated to 70° C. and stirred for 1 h. The reaction was acidified with 1N HCl and diluted with EtOAc (10 mL). The organics were washed with water (1×20 mL), dried over MgSO4, filtered and concentrated. The residue was placed under vacuum and pumped to constant weight to afford 0.015 g (0.02 mmol, 77%) as an off white solid. ES MS m/z 615 (M+H).

### Example 491

### N-{[3-({[(2,6-dimethylphenyl)amino]carbonyl}amino)-2-naphthalenyl]carbonyl}-N-methylglycine

### Step 1

### methyl N-{[3-({[(2,6-dimethylphenyl)amino]carbonyl}amino)-2-naphthalenyl]carbonyl}-N-methylglycinate

HATU (0.34 g, 0.90 mmol) and N,N-diisopropylethylamine (0.16 mL, 0.66 mmol) were added to a solution of 3-({[(2,6-dimethylphenyl)amino]carbonyl}amino)-2-naphthalenecarboxylic acid (0.20 g, 0.60 mmol) in DMF (5 mL). After stirring for 30 min, a solution of methyl N-methylglycinate hydrochloride (0.12 g, 0.90 mmol) in DMF (2 mL) and N,N-diisopropylamine (0.21 mL, 0.90 mmol) were added. The solution was stirred for 12 h then poured into saturated NaHCO₃ and extracted into ethyl acetate. The organic layer was washed with water, dried over MgSO₄, filtered and concentrated. The crude solid was triturated with Et₂O, filtered, and dried under vacuum to give 0.17 g (68%) of the desired product as a white solid.

### Step 2

### N-{[3-({[(2,6-dimethylphenyl)amino]carbonyl}amino)-2-naphthalenyl]carbonyl}-N-methylglycine

A solution of LiOH (0.10 g, 4.17 mmol) in water (2 mL) was added to a solution of methyl N-{[3-({[(2,6-dimethylphenyl)amino]carbonyl}amino)-2-naphthalenyl]carbonyl}-N-methylglycinate (0.18 g, 0.41 mmol) in 1,4-dioxane (5 mL). After stirring for 30 min at RT, 1.0 M HCl and ethyl acetate were added. The organic layer was dried over MgSO₄, filtered and concentrated. The crude material was purified on silica gel (hexanes/ethyl acetate) to give 0.03 g (18%) of the title compound as a white solid. ¹H NMR (DMSO) 400 MHz δ 10.75 (s, 1H), 8.65 (d, 1H), 7.8 (d, 1H), 7.78 (d, 2H), 7.4 (t, 1H), 7.35 (t, 1H), 7.1-6.99 (m, 3H), 3.4-3.2 (m, 2H), 2.83 (s, 3H), 2.2 (s, 6H) ppm.

### Example 492

### 4-({[3-({[(2,6-dimethylphenyl)amino]carbonyl}amino)-2-naphthalenyl]carbonyl}amino)butanoic acid

### Step 1

### ethyl 4-({[3-({[(2,6-dimethylphenyl)amino]carbonyl}amino)-2-naphthalenyl]carbonyl}amino)butanoate

HATU (0.34 g, 0.90 mmol) and N,N-diisopropylethylamine (0.16 mL, 0.67 mmol were added to a solution of 3-({[(2,6-dimethylphenyl)amino]carbonyl}amino)-2-naphthalenecarboxylic acid (0.20 g, 0.60 mmol) in DMF (5 mL). After 30 min, a solution of ethyl 4-aminobutanoate hydrochloride (0.15 g, 0.90 mmol) in DMF (2 mL) and N,N-diisopropylethylamine (0.21 mL, 0.90 mmol) were added. The solution was stirred for 12 h then poured into saturated NaHCO₃ and extracted into ethyl acetate. The organic layer was washed with water, dried over MgSO₄, filtered and concentrated. The crude solid was triturated with Et₂O, filtered, and dried under vacuum to give 0.17 g (63%) of the title compound as a white solid.

### Step 2

### 4-({[3-({[(2,6-dimethylphenyl)amino]carbonyl}amino)-2-naphthalenyl]carbonyl}amino)butanoic acid

A solution of LiOH (0.08 g, 3.33 mmol) in water (2 mL) was added to a solution of ethyl 4-({[3-({[(2,6-dimethylphenyl)amino]carbonyl}amino)-2-naphthalenyl]carbonyl}amino)butanoate (0.15 g, 0.34 mmol) in 1,4-dioxane (5 mL). After stirring for 24 h at RT, 1.0 M HCl and ethyl acetate were added. The organic layer was dried over MgSO₄, filtered and concentrated. The crude solid was triturated with Et₂O, filtered and dried under vacuum to give 0.06 g (44%) of the title compound. ¹H NMR (DMSO) 400 MHz 6 9.85 (s, 1H), 8.9 (s, 1H), 8.6 (s, 1H), 8.2 (s, 1H), 7.8 (d, 1H), 7.7 (d, 1H), 7.45 (t, 1H), 7.39 (t, 1H), 7.1-6.98 (m, 3H), 2.39-2.20 (m, 2H), 2.20-2.0 (m, 8H), 1.8 (s, 2H) ppm.

### Example 493

### methyl N-{[3-({[(2,4,6-trimethylphenyl)amino]carbonyl}amino)-2-naphthalenyl]carbonyl}-beta-alaninate

### Step 1

### 3-({[(2,4,6-trimethylphenyl)amino]carbonyl}amino)-2-naphthalenecarboxylic acid

Triethylamine (0.74 mL, 5.34 mmol) was added to a solution of 3-amino-2-naphthalenecarboxylic acid (0.50 g, 2.67 mmol) in DMF (20 mL). After stirring for 30 min, 2-isocyanato-1,3,5-trimethylbenzene (0.47 g, 2.93 mmol) was added and the solution heated to 75° C. for 3 h. The reaction was cooled to RT and then 11.0M HCl and ethyl acetate were added. The organic layer was concentrated and the resulting solid was washed with Et₂O, filtered, and dried under vacuum to give 0.61 g (65% yield) of the title compound as a tan solid.

### Step 2

### methyl N-{[3-({[(2,4,6-trimethylphenyl)amino]carbonyl}amino)-2-naphthalenyl]carbonyl}-beta-alaninate

HATU (0.33 g, 0.87 mmol) and N,N-diisopropylethylamine (0.15 mL, 0.86 mmol) were added to a solution of 3-({[(2,4,6-trimethylphenyl)amino]carbonyl}amino)-2-naphthalenecarboxylic acid (0.20 g, 0.57 mmol) in DMF (5 mL). After 30 min, a solution of methyl beta-alaninate hydrochloride (0.15 g, 0.90 mmol) in DMF (2 mL) and N,N-diisopropylethylamine (0.16 mL, 0.90 mmol) were added. The solution was stirred for 3 h then poured into saturated NaHCO₃ and extracted into ethyl acetate. The organic layer was washed with water, dried over MgSO₄, filtered and concentrated. The solid was triturated with Et₂O, filtered and dried under vacuum to give 0.18 g (72%) of the title compound as a white solid. ¹H NMR (DMSO) 400 MHz delta 9.8 (s, 1H), 8.95 (s, 1H), 8.6 (s, I H), 8.1 (s, 1H), 7.8 (d, 1H), 7.75 (d, 1H), 7.5 (t, 1H), 7.39 (t, 1H), 6.9 (s, 2H), 3.6 (s, 2H), 3.3 (s, 3H), 2.65 (s, 2H), 2.22 (s, 3H), 2.15 (s, 6H) ppm.

### Example 494

### N-{[3-({[(2,4,6-trimethylphenyl)amino]carbonyl}amino)-2-naphthalenyl]carbonyl}-beta-alanine

A solution of LiOH (0.10 g, 3.46 mmol) in water (2 mL) was added to a solution of methyl N-{[3-({[(2,4,6-trimethylphenyl)amino]carbonyl}amino)-2-naphthalenyl]carbonyl}-beta-alaninate (0.15 g, 0.34 mmol) in 1,4-dioxane (5 mL). After stirring for 3 h at RT, 1.0 M HCl and ethyl acetate were added. The organic layer was dried over MgSO₄, filtered and concentrated. The crude product was purified on silica gel (hexanes/ethyl acetate) to give 0.025 g (18%) of the title compound as a white solid. ¹H NMR (DMSO) 400 MHz delta 12.3 (s, 1H), 9.8 (s, m 1H), 8.9 (s, 1H), 8.8 (s, 1H), 8.1 (s, 1H), 7.8 (d, 1H), 7.7 (d, 1H), 7.5 (t, 1H), 7.39 (t, 1H), 6.9 (s, 2H), 3.5 (s, 2H), 2.6 (s, 2H), 2.2 (s, 3H), 2.19 (s, 6H) ppm.

### Example 495

### 3-({[3-({[(2,4,6-trimethylphenyl)amino]carbonyl}amino)-2-naphthalenyl]carbonyl}amino)benzoic acid

### Step 1

### 1,1-dimethylethyl 3-({[3-({[(2,4,6-trimethylphenyl)amino]carbonyl}amino)-2-naphthalenyl]carbonyl}amino)benzoate

HATU (0.24 g, 0.64 mmol) and N,N-diisopropylethylamine (0.11 mL, 0.64 mmol) were added to a solution of 3-({[(2,4,6-trimethylphenyl)amino]carbonyl}amino)-2-naphthalenecarboxylic acid (0.15 g, 0.43 mmol) in DMF (5 mL). After 30 min, a solution of 1,1-dimethylethyl 3-aminobenzoate (0.15 g, 0.90 mmol) in DMF (2 mL) was added. The solution was stirred for 4 h at RT then poured into saturated NaHCO₃ and extracted into ethyl acetate. The organic layer was washed with water, dried over MgSO₄, filtered and concentrated. The solid was triturated with Et₂O, filtered and dried under vacuum to give 0.17 g (75%) of the title compound.

### Step 2

### 3-({[3-({[(2,4,6-trimethylphenyl)amino]carbonyl}amino)-2-naphthalenyl]carbonyl}amino)benzoic acid

TFA (2 mL) was added to a solution of 1,1-dimethylethyl 3-({[3-({[(2,4,6-trimethylphenyl)amino]carbonyl}amino)-2-naphthalenyl]carbonyl}amino)benzoate (0.17 g, 0.32 mmol) in DCM (5 mL). After stirring for 12 h at RT, the solution was concentrated to dryness. The resulting solid was triturated with Et₂O, filtered and dried under vacuum to give 0.025 g (18%) of the desired product as a white solid. ¹H NMR (DMSO) 400 MHz delta 12.8 (s, 1H), 10.85 (s, 1H), 9.4 (s, 1H), 8.6 (s, 1H), 8.37 (s, 1H), 7.98 (d, 2H), 7.8 (d, 2H), 7.7 (d, 2H), 7.58-7.4 (m, 3H), 6.9 (s, 2H), 2.2 (s, 3H), 2.15 (s, 6H) ppm.

### Example 496

### N-{[3-({[(2,4,6-trimethylphenyl)amino]carbonyl}amino)-2-naphthalenyl]carbonyl}-L-valine

### Step 1

### 3-({[(2,4,6-trimethylphenyl)amino]carbonyl}amino)-2-naphthalenecarboxylic acid

Triethylamine (4.46 mL, 32 mmol) was added to a solution of 3-amino-2-naphthalenecarboxylic acid (3.00 g, 16.04 mmol) in DMF (100 mL). After stirring for 30 min, 2-isocyanato-1,3,5-trimethylbenzene (2.83 g, 17.60 mmol) was added and the solution heated to 75° C. for 3 h. The reaction was cooled to RT and then 1.0M HCl and ethyl acetate were added. The organic layer was dried over MgSO₄, filtered and concentrated. The resulting crude residue was washed with Et₂O and the solid was filtered and dried under vacuum to give 4.2 g (75%) of the title compound as a cream solid.

### Step 2

### methyl N-{[3-({[(2,4,6-trimethylphenyl)amino]carbonyl}amino)-2-naphthalenyl]carbonyl}-L-valinate

HATU (0.26 g, 0.68 mmol) and N,N-diisopropylethylamine (0.12 mL, 0.68 mmol) were added to a solution of 3-({[(2,4,6-trimethylphenyl)amino]carbonyl}amino)-2-naphthalenecarboxylic acid (0.20 g, 0.57 mmol) in DMF (3 mL). After 30 min, a solution of methyl L-valinate hydrochloride(0.15 g, 0.90 mmol) in DMF (2 mL) and N,N-diisopropylethylamine (0.1 mL, 0.57 mmol) was added. The solution was stirred for 4 h at RT then poured into saturated NaHCO₃ and extracted into ethyl acetate. The organic layer was washed with water, dried over MgSO₄, filtered and concentrated. The crude material was purified on silica gel (hexanes/ethyl acetate) to give 0.18 g (70%) of the title compound.

### Step 3

### N-{[3-({[(2,4,6-trimethylphenyl)amino]carbonyl}amino)-2-naphthalenyl]carbonyl}-L-valine

A solution of LiOH (0.1 g, 3.9 mmol) in water (2 mL) was added to a solution of methyl N-{[3-({[(2,4,6-trimethylphenyl)amino]carbonyl}amino)-2-naphthalenyl]carbonyl}-L-valinate (0.18 g, 0.39 mmol) in 1,4-dioxane (5 mL). After stirring for 3 h at RT, 1.0 M HCl and ethyl acetate were added. The organic layer was dried over MgSO₄, filtered and concentrated to give 0.010 g (7%) of the title compound. ¹H NMR (DMSO) 400 MHz delta 12.8 (s, 1H), 9.6 (s, 1H), 8.9 (s, 1H), 8.8 (s, 1H), 8.2 (s, 1H), 7.9 (d, 1H), 7.79 (d, 1H), 7.5 (t, 1H), 7.4 (t, 1H), 6.9 (s, 2H), 4.5 (s, 1H), 2.3-2.0 (m, 10H), 1.0 (s, 6H) ppm.

### Example 497

### 4-({[3-({[(2,6-dichlorophenyl)amino]carbonyl}amino)-2-naphthalenyl]carbonyl}amino)-3-thiophenecarboxylic acid

### Step 1

### 3-({[(2,6-dichlorophenyl)amino]carbonyl}amino)-2-naphthalenecarboxylic acid

Triethylamine (4.4 mL, 32.0 mmol) was added to a solution of 3-amino-2-naphthalenecarboxylic acid (3.00 g, 16.04 mmol) in DMF (100 mL). After stirring for 30 min, 1,3-dichloro-2-isocyanatobenzene (3.3 g, 17.6 mmol) was added and the solution heated to 75° C. for 3 h. The reaction was cooled to RT and then 1.0M HCl was added. The resulting precipitate was filtered, washed with ethyl acetate and Et₂O then dried under vacuum to give 4.5 g (75%) of the title compound as a cream solid.

### Step 2

### methyl 4-({[3-({[(2,6-dichlorophenyl)amino]carbonyl}amino)-2-naphthalenyl]carbonyl}amino)-3-thiophenecarboxylate

HATU (0.38 g, 1.00 mmol) and N,N-diisopropylethylamine (0.73 mL, 4.20 mmol) were added to a solution of 3-({[(2,6-dichlorophenyl)amino]carbonyl}amino)-2-naphthalenecarboxylic acid (0.25 g, 0.67 mmol) in DCM (5 mL) and DMF (1 mL). After 30 min, a solution of methyl 4-amino-3-thiophenecarboxylate (0.17 g, 1.08 mmol) in DMF (3 mL) was added. The solution was stirred for 24 h at RT then poured into saturated NaHCO₃ and extracted into ethyl acetate. The organic layer was washed with water, dried over MgSO₄, filtered and concentrated. The crude material was purified on silica gel using an ISCO chromatography system (100% hexanes to 30% ethyl acetate/hexanes over 30 min) to give 0.17 g (50%) of the title compound.

### Step 3

### 4-({[3-({[(2,6-dichlorophenyl)amino]carbonyl}amino)-2-naphthalenyl]carbonyl}amino)-3-thiophenecarboxylic acid

A solution of LiOH (0.08 g, 3.33 mmol) in water (2 mL) was added to a solution of methyl 4-({[3-({[(2,6-dichlorophenyl)amino]carbonyl}amino)-2-naphthalenyl]carbonyl}amino)-3-thiophenecarboxylate (0.17 g, 0.33 mmol) in 1,4-dioxane (2 mL) and water (1 mL). After stirring for 3 h at RT, 1.0 M HCl and ethyl acetate were added. The organic layer was dried over MgSO₄, filtered and concentrated to give 0.06 g (37%) of the desired product as a white solid. ¹H NMR (DMSO) 400 MHz delta 11.25 (2, 1H), 9.8 (s, I H), 9.4 (s, I H), 8.67 (s, 1H), 8.4 (d, 2H), 8.0 (s, 1H), 7.97 (d, 1H), 7.8 (d, 1H), 7.6-7.5 (m, 2H), 7.5 (t, I H), 7.38 (t, I H) ppm.

### Example 498

### 5-({[3-({[(2,4,6-trimethylphenyl)amino]carbonyl}amino)-2-naphthalenyl]carbonyl}amino)-1,3-benzenedicarboxylic acid

### Step 1

### dimethyl 5-({[3-({[(2,4,6-trimethylphenyl)amino]carbonyl}amino)-2-naphthalenyl]carbonyl}amino)-1,3-benzenedicarboxylate

HATU (0.41 g, 1.08 mmol) and N,N-diisopropylethylamine (0.14 mL, 0.81 mmol) were added to a solution of 3-({[(2,4,6-trimethylphenyl)amino]carbonyl}amino)-2-naphthalenecarboxylic acid (0.25 g, 0.72 mmol) in DMF (1 mL). After 30 min, a solution of dimethyl 5-amino-1,3-benzenedicarboxylate (0.22 g, 1.08 mmol) in DCM (4 mL) was added. The solution was stirred for 4 h at RT then poured into saturated NaHCO₃ and extracted into ethyl acetate. The organic layer was washed with water, dried over MgSO₄, filtered and concentrated. The crude material was purified on silica gel using an ISCO chromatography system (ethyl acetate/hexanes) to give 0.08 g (21%) of the title compound.

### Step 2

### 5-({[3-({[(2,4,6-trimethylphenyl)amino]carbonyl}amino)-2-naphthalenyl]carbonyl}amino)-1,3-benzenedicarboxylic acid

A solution of LiOH (0.04 g, 1.50 mmol) in water (2 mL) was added to a solution of dimethyl 5-({[3-({[(2,4,6-trimethylphenyl)amino]carbonyl}amino)-2-naphthalenyl]carbonyl}amino)-1,3-benzenedicarboxylate (0.08 g, 0.15 mmol) in 1,4-dioxane (2 mL) and water (1 mL). After stirring for 3 h at RT, 1.0 M HCl and ethyl acetate were added. The organic layer was dried over MgSO₄, filtered and concentrated. The crude solid was triturated with Et₂O to give 0.01 g (13%) of the title compound. ES-MS M/Z 512 (M+H).

### Example 499

### 1-({[3-({[(2,4,6-trimethylphenyl)amino]carbonyl}amino)-2-naphthalenyl]carbonyl}amino)cyclopropanecarboxylic acid

### Step 1

### methyl 1-{[(3-amino-2-naphthalenyl)carbonyl]amino}cyclopropanecarboxylate

HATU (1.5 g, 3.9 mmol) and N,N-diisopropylethylamine (0.68 g, 4.05 mmol) were added to a solution of 3-amino-2-naphthalenecarboxylic acid (0.5 g, 2.7 mmol) in DMF (5 mL). After 30 min, a solution of methyl 1-aminocyclopropanecarboxylate (0.22 g, 1.08 mmol) in DCM (2 mL) was added. The solution was stirred for 4 h at RT then poured into saturated NaHCO₃ and extracted into ethyl acetate. The organic layer was washed with water, dried over MgSO₄, filtered and concentrated. The crude material was purified on silica gel using an ISCO chromatography system (ethyl acetate/hexanes) to give 0.45 g (60%) of the title compound.

### Step 2

### methyl 1-({[3-({[(2,4,6-trimethylphenyl)amino]carbonyl}amino)-2-naphthalenyl]carbonyl}amino)cyclopropanecarboxylate

To a pyridine solution (5 mL) containing methyl 1-{[(3-amino-2-naphthalenyl)carbonyl]amino}cyclopropanecarboxylate (0.22 g, 0.77 mmol) was added 2-isocyanato-1,3,5-trimethylbenzene (0.37 g, 2.31 mmol). The solution was stirred at RT for approximately 12 h then the pyridine was removed in vacuo. The residue was dissolved in ethyl acetate and the resulting precipitate was removed by filtration. The organic layer was washed with 1.0M HCl, dried over MgSO₄, and concentrated. The crude material was purified on silica gel using an ISCO chromatography system (0-40% hexane/ethyl acetate) to give 0.17 g (50%) of the title compound as a white solid.

### Step 3

### 2-({[3-({[(2,4,6-trimethylphenyl)amino]carbonyl}amino)-2-naphthalenyl]carbonyl}amino)cyclopropanecarboxylic acid (u21828/49)

A solution of LiOH (0.09 g, 3.80 mmol) in water (2 mL) was added to a solution of methyl 1-({[3-({[(2,4,6-trimethylphenyl)amino]carbonyl}amino)-2-naphthalenyl]carbonyl}amino)cyclopropanecarboxylate (0.17 g, 0.38 mmol) in 1,4-dioxane (2 mL) and water (1 mL). After stirring for 3 h at RT, 1.0 M HCl and ethyl acetate were added. The organic layer was dried over MgSO₄, filtered and concentrated. The crude solid was triturated with Et₂O to give 0.03 g (18%) of the title compound. ¹H NMR (DMSO) 400 MHz delta 12.6 (s, 1H), 10.2 (s, 1H), 9.4 (s, 1H), 8.63 (s, 1H), 8.25 (s, 1H), 7.80 (d, 1H), 7.78 (d, 1H), 7.5 (t, 1H), 7.4 (t, 1H), 6.9 (s, 2H), 2.2 (s, 3H), 2.18 (s, 6H), 1.5 (s, 2H), 1.2 (s, 2H) ppm.

### Example 500

### 3-({[3-({[(2,6-dimethylphenyl)amino]carbonyl}amino)-2-naphthalenyl]carbonyl}amino)butanoic acid

### Step 1

### ethyl 3-({[3-({[(2,6-dimethylphenyl)amino]carbonyl}amino)-2-naphthalenyl]carbonyl}amino)butanoate

HATU (0.26 g, 0.68 mmol) and N,N-diisopropylethylamine (0.11 mL, 0.67 mmol) were added to a solution of 3-({[(2,6-dimethylphenyl)amino]carbonyl}amino)-2-naphthalenecarboxylic acid (0.15 g, 0.45 mmol) in DMF (3 mL). After 30 min, a solution of ethyl 3-aminobutanoate (0.1 g, 0.7 mmol) in DMF (2 mL) was added. The solution was stirred for 4 h at RT then poured into saturated NaHCO₃ and extracted into ethyl acetate. The organic layer was washed with water, dried over MgSO₄, filtered and concentrated. The crude material was purified on silica gel using an ISCO chromatography system (ethyl acetate/hexanes) to give 0.06 g (30%) of the title compound.

### Step 2

### 3-({[3-({[(2,6-dimethylphenyl)amino]carbonyl}amino)-2-naphthalenyl]carbonyl}amino)butanoic acid

A solution of LiOH (0.03 g, 1.3 mmol) in water (1 mL) was added to a solution of ethyl 3-({[3-({[(2,6-dimethylphenyl)amino]carbonyl}amino)-2-naphthalenyl]carbonyl}amino)butanoate (0.06 g, 0.13 mmol) in 1,4-dioxane (2 mL) and water (1 mL). After stirring for 3 h at RT, 1.0 M HCl and ethyl acetate were added. The organic layer was dried over MgSO₄, filtered and concentrated. The crude solid was triturated with Et₂O and CHCl₃ to give 0.02 g (37%) of the title compound. ¹H NMR (DMSO) 400 MHz delta 8.5 (d, 1H), 8.0 (s, 1H), 7.74 (d, 1H), 7.52 (d, 1H), 7.36-7.23 (m, 1H), 7.19 (m, 1H), 6.97 (s, 1H), 6.08 (s, 2H), 4.42-4.29 (m, 1H), 4.14-3.95 (m, 2H), 3.38 (s, 3H), 2.5 (s, 6H) ppm.

### Example 501

### (2S)-(4-hydroxyphenyl)({[3-({[(2,4,6-trimethylphenyl)amino]carbonyl}amino)-2-naphthalenyl]carbonyl}amino)ethanoic acid

### Step 1

### methyl(2S)-{[(3-amino-2-naphthalenyl)carbonyl]amino}(4-hydroxyphenyl)ethanoate

HATU (0.76 g, 2.00 mmol) and N,N-diisopropylethylamine (0.34 mL, 1.96 mmol) were added to a solution of 3-amino-2-naphthalenecarboxylic acid (0.25 g, 1.33 mmol) in DMF (1.5 mL) and DCM (1.5 mL). After 30 min, a solution of methyl(2S)-amino(4-hydroxyphenyl)ethanoate (0.1 g, 0.7 mmol) in DMF (2 mL) was added. The solution was stirred for 4 h at RT then poured into saturated NaHCO₃ and extracted into DCM. The organic layer was washed with water, dried over MgSO₄, filtered and concentrated. The crude material was purified on silica gel using an ISCO chromatography system (ethyl acetate/hexanes) to give 0.15 g (32%) of the title compound.

### Step 2

### methyl(2S)-(4-hydroxyphenyl)({[3-({[(2,4,6-trimethylphenyl)amino]carbonyl}amino)-2-naphthalenyl]carbonyl}amino)ethanoate

To a pyridine solution (15 mL) containing methyl(2S)-{[(3-amino-2-naphthalenyl)carbonyl]amino}(4-hydroxyphenyl)ethanoate (0.15 g, 0.43 mmol) was added 2-isocyanato-1,3,5-trimethylbenzene (0.34 g, 2.11 mmol). The solution was stirred for 12 h at RT then the pyridine was removed in vacuo. The residue was dissolved in ethyl acetate and the resulting precipitate was removed by filtration. The organic layer was washed with 1.0M HCl, dried over MgSO₄, and concentrated. The crude material was purified on silica gel using an ISCO chromatography system (0-40% hexane/ethyl acetate) to give 0.05 g (23% yield) of the title compound.

### Step 3

### (2S)-(4-hydroxyphenyl)({[3-({[(2,4,6-trimethylphenyl)amino]carbonyl}amino)-2-naphthalenyl]carbonyl}amino)ethanoic acid

A solution of LiOH (0.02 g, 0.97 mmol) in water (2 mL) was added to a solution of methyl(2S)-(4-hydroxyphenyl)({[3-({[(2,4,6-trimethylphenyl)amino]carbonyl}amino)-2-naphthalenyl]carbonyl}amino)ethanoate (0.05 g, 0.10 mmol) in 1,4-dioxane (5 mL). After stirring for 3 h at RT, 1.0 M HCl and ethyl acetate were added. The organic layer was dried over MgSO₄, filtered and concentrated. The crude product was purified on a reverse phase HPLC (Gilson: MeCN, 1% TFA/water) to give 0.001 g (20%) of the title compound. ES-MS M/Z 496 (M−H).

### Example 502

### (2S)-(4-hydroxycyclohexyl)({[3-({[(2,4,6-trimethylphenyl)amino]carbonyl}amino)-2-naphthalenyl]carbonyl}amino)ethanoic acid

### Step 1

### methyl(2S)-{[(3-amino-2-naphthalenyl)carbonyl]amino}(4-hydroxycyclohexyl)ethanoate

HATU (1.21 g, 3.20 mmol) and N,N-diisopropylethylamine (0.56 mL, 3.21 mmol) were added to a solution of 3-amino-2-naphthalenecarboxylic acid (0.50 g, 2.67 mmol) in DMF (2.5 mL) and DCM (2.5 mL). After 30 min, a solution of (2S)-amino(4-hydroxycyclohexyl)ethanoic acid (0.6 g, 3.2 mmol) in DCM (2 mL) was added. The solution was stirred for 4 h at RT then poured into saturated NaHCO₃ and extracted into DCM. The organic layer was washed with water, dried over MgSO₄, filtered and concentrated. The crude material was purified on silica gel using an ISCO chromatography system (ethyl acetate/hexanes) to give 0.37 g (41%) of the title compound.

### Step 2

### methyl(2S)-(4-hydroxycyclohexyl)({[3-({[(2,4,6-trimethylphenyl)amino]carbonyl}amino)-2-naphthalenyl]carbonyl}amino)ethanoate

To a pyridine solution (5 mL) containing methyl(2S)-{[(3-amino-2-naphthalenyl)carbonyl]amino}(4-hydroxycyclohexyl)ethanoate (0.15 g, 0.42 mmol) was added 2-isocyanato-1,3,5-trimethylbenzene (0.2 g, 1.2 mmol). The solution was stirred for 12 h at RT then the pyridine was removed in vacuo. The residue was dissolved in ethyl acetate and the resulting precipitate was removed by filtration. The organic layer was washed with 1.0M HCl, dried over MgSO₄, and concentrated. The crude material was purified on silica gel using an ISCO chromatography system (0-40% ethyl acetate/hexanes) to give 0.065 g (30%) of the title compound.

### Step 3

### (2S)-(4-hydroxycyclohexyl)({[3-({[(2,4,6-trimethylphenyl)amino]carbonyl}amino)-2-naphthalenyl]carbonyl}amino)ethanoic acid

A solution of LiOH (0.03 g, 1.25 mmol) in water (1 mL) was added to a solution of methyl(2S)-(4-hydroxycyclohexyl)({[3-({[(2,4,6-trimethylphenyl)amino]carbonyl}amino)-2-naphthalenyl]carbonyl}amino)ethanoate (0.06 g, 0.12 mmol) in THF (5 mL) and MeOH (1 mL). After stirring for 12 h at RT, 1.0 M HCl and ethyl acetate were added. The organic layer was dried over MgSO₄, filtered and concentrated. The crude solid was triturated with Et₂O and dried to give 0.20 (34%) of the title compound. ES-MS M/Z 502 (M−H).

### Example 503

### methyl N⁴,N⁴-dimethyl-N²-{[4′-(methyloxy)-3-({[(2,4,6-trimethylphenyl)amino]carbonyl}amino)-4-biphenylyl]carbonyl}-L-asparaginate

A solution of HATU (0.61 g, 1.60 mmol) and (3S)-4-(methyloxy)-3-({[4′-(methyloxy)-3-({[(2,4,6-trimethylphenyl)amino]carbonyl}amino)-4-biphenylyl]carbonyl}amino)-4-oxobutanoic acid (0.28 g, 0.53 mmol) in DCM (10 mL) were stirred at RT for 5 min then a 2M solution of dimethylamine (1.6 mL, 3.2 mmol) in THF was added. After stirring at RT for 2 h, ethyl acetate (100 mL) and saturated aqueous NaHCO₃ solution (100 mL) were added. The organic layer was washed with saturated aqueous NaHCO₃ solution (100 mL), brine (200 mL), dried over MgSO₄, filtered and concentrated. The crude oil was purified on silica gel (ISCO: eluting with 100% hexanes to 100% ethyl acetate over 40 min) to give 0.23 g (77%) of the title compound as a white powder. APCI m/z 561 (M+H).

### Example 504

### N⁴,N⁴-dimethyl-N²-{[4′-(methyloxy)-3-({[(2,4,6-trimethylphenyl)amino]carbonyl}amino)-4-biphenylyl]carbonyl}-L-asparagine

A solution of LiOH (0.058 g, 2.43 mmol) in hot water (3 mL) was added to a solution of methyl N⁴,N⁴-dimethyl-N²-{[4′-(methyloxy)-3-({[(2,4,6-trimethylphenyl)amino]carbonyl}amino)-4-biphenylyl]carbonyl}-L-asparaginate (0.17 g, 0.30 mmol) in THF (5 mL) and MeOH (3 mL). After stirring at RT for 1 h, the THF was removed via rotary evaporation then 1N HCl (20 mL) was added to the remaining aqueous layer. The resulting white slurry was filtered, washed with water (20 mL), then dissolved in ethyl acetate (50 mL) and dried over MgSO₄. The organic layer was filtered, concentrated via rotary evaporation and dried under vacuum. The crude material was dissolved in DCM (2 mL) and Et₂O (5 ml) was added. The white solid was filtered and dried under vacuum to give 0.077 g (46%) of the title compound as a white powder. APCI m/z 547 (M+H).

### Example 505

### N-({3-[({[4-(cyclopropylmethyl)-2,6-dimethylphenyl]amino}carbonyl)amino]-3′-fluoro-4-biphenylyl}carbonyl)-O-(1,1-dimethylethyl)-L-threonine

### Step 1

### methyl 3′-fluoro-3-nitro-4-biphenylcarboxylate

Five 20 ml microwave reaction vials were each charged with methyl 4-chloro-2-nitrobenzoate (1.00 g, 4.64 mmol), (3-fluorophenyl)boronic acid (0.71 g, 5.10 mmol), cesium fluoride (2.12 g, 13.92 mmol) and Pd(Cy₃)₂Cl₂ (0.17 g, 0.23 mmol) in MeCN (12 mL) and water (2 mL). The vials were sealed and heated to 1500° C. for 5 min in a Microwave Reactor. The vials were carefully vented then combined in a separatory funnel, diluted with ethyl acetate (300 mL), washed with water (200 mL), brine (200 mL), dried over MgSO₄, filtered and concentrated. The crude oil was purified on silica gel (ISCO: eluting with 100% hexanes to 100% ethyl acetate over 50 min) to give 5.64 g (89%) of the title compound as a yellow oil.

### Step 2

### 3′-fluoro-3-nitro-4-biphenylcarboxylic acid

A solution of LiOH (1.48 g, 61.48 mmol) in hot water (40 mL) was added to a solution of methyl 3′-fluoro-3-nitro-4-biphenylcarboxylate (5.64 g, 20.49 mmol) in THF (100 mL) and MeOH (30 mL). After stirring at RT for 1 h, the THF was removed via rotary evaporation then 1N HCl (75 mL) was added to the remaining aqueous layer. The resulting white slurry was filtered, washed with water (20 mL) and dried under vacuum to give 4.30 g (80%) of the title compound as a white powder.

### Step 3

### methyl O-(1,1-dimethylethyl)-N-[(3′-fluoro-3-nitro-4-biphenylyl)carbonyl]-L-threoninate

HATU (2.91 g, 7.66 mmol) was added to a suspension of 3′-fluoro-3-nitro-4-biphenylcarboxylic acid (1.00 g, 3.83 mmol) in DCM (25 mL). After 5 min, N,N-diisopropylethylamine (1.33 mL, 7.66 mmol) was added, followed by methyl O-(1,1-dimethylethyl)-L-threoninate hydrochloride (1.29 g, 5.74 mmol). The solution was stirred at RT for 3 h then saturated aqueous NaHCO₃ solution (100 mL) and ethyl acetate (150 mL) were added. The organic layer was washed with saturated aqueous NaHCO₃ solution (100 mL), brine (200 mL), dried over MgSO₄, filtered and concentrated. The crude oil was purified on silica gel (ISCO: eluting with 100% hexanes to 60% ethyl acetate over 40 min) to give 1.58 g (95%) of the title compound as a white gummy powder. APCI m/z 433 (M+H).

### Step 4

### methyl N-[(3-amino-3′-fluoro-4-biphenylyl)carbonyl]-O-(1,1-dimethylethyl)-L-threoninate

A mixture of methyl O-(1,1-dimethylethyl)-N-[(3′-fluoro-3-nitro-4-biphenylyl)carbonyl]-L-threoninate (1.52 g, 3.51 mmol) and 10% Pd/C (0.15 g) in ethyl acetate (30 mL) and MeOH (15 mL) was stirred under hydrogen (60 psig) at RT for 16 h. The next day the reaction was carefully vented, diluted with ethyl acetate, and filtered through Celite to give 1.26 g (83%) of the title compound as a white gummy powder. APCI m/z 403 (M+H).

### Step 5

### methyl N-{[3-({[(4-bromo-2,6-dimethylphenyl)amino]carbonyl}amino)-3′-fluoro-4-biphenylyl]carbonyl}-O-(1,1-dimethylethyl)-L-threoninate

A solution of methyl N-[(3-amino-3′-fluoro-4-biphenylyl)carbonyl]-O-(1,1-dimethylethyl)-L-threoninate (0.59 g, 1.47 mmol) and 5-bromo-2-isocyanato-1,3-dimethylbenzene (0.67 g, 2.95 mmol) in pyridine (8 mL) were stirred at RT for 16 h then concentrated to dryness via rotary evaporation. An aqueous 1N HCl solution (50 mL) and ethyl acetate (150 mL) were added. The organic layer was washed with saturated aqueous NaHCO₃ solution (100 mL), brine (200 mL), dried over MgSO₄, filtered and concentrated to give 0.92 g (99%) of the title compound as a white solid. APCI m/z 626 (M−H).

### Step 6

### methyl O-(1,1-dimethylethyl)-N-({3-[({[2,6-dimethyl-4-(2-propen-1-yl)phenyl]amino}carbonyl)amino]-3′-fluoro-4-biphenylyl}carbonyl)-L-threoninate

A mixture of methyl N-{[3-({[(4-bromo-2,6-dimethylphenyl)amino]carbonyl}amino)-3′-fluoro-4-biphenylyl]carbonyl}-O-(1,1-dimethylethyl)-L-threoninate (0.92 g, 1.46 mmol), allyltributylstannane (0.53 g, 1.61 mmol) and tetrakispalladium(trphenylphosphine) (0.085 g, 0.070 mmol) in MeCN (10 mL) were heated to 1500° C. for 30 min in a microwave reactor. Upon cooling to RT, the mixture was poured into a separatory funnel containing water (100 mL) and ethyl acetate (150 mL). The organic layer was washed with brine (200 mL), dried over MgSO₄, filtered and concentrated. The crude material was purified on silica gel using an ISCO chromatography system (increasing solvent gradient from 100% hexanes to 90% ethyl acetate (hexanes over 30 min) to give 0.19 g (22%) of the title compound as a clear oil solid. APCI m/z 590 (M+H).

### Step 7

### methyl N-({3-[({[4-(cyclopropylmethyl)-2,6-dimethylphenyl]amino}carbonyl)amino]-3′-fluoro-4-biphenylyl}carbonyl)-O-(1,1-dimethylethyl)-L-threoninate

N-methyl-N-nitrosourea (0.33 g, 3.22 mmol) was added as a solid to a mixture of 30% aqueous KOH (32 mL) and DCM (25 mL) at 0° C. After stirring for 5 min, the mixture was poured into a smooth separatory funnel (ie no chips, cracks, etc) with a Teflon stopcock. The yellow organic layer was separated and dried over KOH pellets at 0° C. Half of the solution was added to a smooth addition funnel with a Teflon stopcock and added dropwise to a mixture of methyl O-(1,1-dimethylethyl)-N-({3-[({[2,6-dimethyl-4-(2-propen-1-yl)phenyl]amino}carbonyl)amino]-3′-fluoro-4-biphenylyl}carbonyl)-L-threoninate (0.19 g, 0.32 mmol) and Pd(acac)₂ (0.01 g, 0.03 mmol) in Et₂O at 0° C. Upon addition, the remaining diazomethane solution was added to the separatory funnel and added dropwise. Upon complete addition, the solution was warmed to RT and stirred for 0.5 h. The pale yellow solution was filtered through Celite and concentrated. The crude yellow oil was purified on silica gel using an ISCO chromatography system (increasing solvent gradient from 100% hexanes to 90% ethyl acetate/hexanes over 30 min) to give 0.153 (79%) of the title compound as a pale yellow oil. APCI m/z 604 (M+H).

### Step 8

### N-({3-[({[4-(cyclopropylmethyl)-2,6-dimethylphenyl]amino}carbonyl)amino]-3′-fluoro-4-biphenylyl}carbonyl)-O-(1,1-dimethylethyl)-L-threonine

A hot solution of LiOH (0.089 g, 3.73 mmol) in water (5 mL) was added to a solution of methyl N-({3-[({[4-(cyclopropylmethyl)-2,6-dimethylphenyl]amino}carbonyl)amino]-3′-fluoro-4-biphenylyl}carbonyl)-(1,1-dimethylethyl)-L-threoninate (0.15 g, 0.25 mmol) in THF (5 mL) and MeOH (5 mL). After 3 h, the organic solvent was removed under reduced pressure then 1N HCl solution (5 mL) and ethyl acetate (50 mL) were added. The organic layer was washed with brine, dried over MgSO₄, filtered and concentrated. The crude product was dissolved in a minimal amount of hot ethyl acetate (ca. 5 mL) then hexanes (50 mL) was added. The white precipitate was filtered (gummy solid), dissolved in ethyl acetate and DCM, and concentrated to give 0.105 g (72%) of the title compound as a white solid. APCI m/z 588 (M−H).

### Example 506

### (2S)-4-(ethyloxy)-2-({[3′-fluoro-3-({[(2,4,6-trimethylphenyl)amino]carbonyl}amino)-4-biphenylyl]carbonyl}amino)-4-oxobutanoic acid

### Step 1

### 4-ethyl 1-(phenylmethyl) N-[(3′-fluoro-3-nitro-4-biphenylyl)carbonyl]-L-aspartate

HATU (1.16 g, 3.06 mmol) was added to a suspension of 3′-fluoro-3-nitro-4-biphenylcarboxylic acid (0.40 g, 1.53 mmol) in DCM (10 mL). After 5 min, N,N-diisopropylethylamine (0.54 mL, 3.06 mmol) was added, followed by 4-ethyl 1-(phenylmethyl) L-aspartate trifluoroacetate (0.84 g, 2.30 mmol). The solution was stirred at RT for 16 h then saturated aqueous NaHCO₃ solution (100 mL) and ethyl acetate (150 mL) were added. The organic layer was washed with aqueous 1N HCl solution (2 x's 50 mL), saturated aqueous NaHCO₃ solution (100 mL), brine (200 mL), dried over MgSO₄, filtered and concentrated. The crude oil was purified on silica gel (ISCO: eluting with 100% hexanes to 100% ethyl acetate over 40 min) to give 0.227 g (30%) of the title compound as a white powder. APCI m/z 494 (M−H).

### Step 2

### 4-ethyl 1-(phenylmethyl) N-{[3′-fluoro-3-({[(2,4,6-trimethylphenyl)amino]carbonyl}amino)-4-biphenylyl]carbonyl}-L-aspartate

A mixture of 4-ethyl 1-(phenylmethyl) N-[(3′-fluoro-3-nitro-4-biphenylyl)carbonyl]-L-aspartate (0.23 g, 0.47 mmol) and Pt(sulfided) (0.10 g) in MeOH (15 mL) was stirred under a balloon of hydrogen (1 atmosphere) at RT for 16 h. The next day the reaction was carefully vented, diluted with ethyl acetate, and filtered through Celite to give the title compound as a white gummy powder. LC/MS shows multiple peaks; used as is in next step. The crude product was dissolved in pyridine (8 mL) and 2-isocyanato-1,3,5-trimethylbenzene (0.15 g, 0.94 mmol) was added. The mixture was stirred for 16 h then concentrated to dryness. An aqueous 1N HCL solution (50 mL) and ethyl acetate (100 mL) were added. The organic layer was washed with saturated NaHCO₃ solution (50 mL), brine (50 mL), dried over MgSO₄, filtered and concentrated. The crude product was purified on silica gel using an ISCO chromatography system (increasing solvent gradient from 100% hexanes to 90% ethyl acetate/hexanes over 30 min) to give 0.094 g (32%) of the title compound as a white solid. APCI m/z 626 (M+H).

### Step 3

### (2S)-4-(ethyloxy)-2-({[3′-fluoro-3-({[(2,4,6-trimethylphenyl)amino]carbonyl}amino)-4-biphenylyl]carbonyl}amino)-4-oxobutanoic acid

A mixture of 4-ethyl 1-(phenylmethyl) N-{[3′-fluoro-3-({[(2,4,6-trimethylphenyl)amino]carbonyl}amino)-4-biphenylyl]carbonyl}-L-aspartate (0.094 g, 0.15 mmol) and 10% Pd/C (0.060 g) in MeOH (5 mL) and ethyl acetate (5 mL) were stirred at RT under 1 atmosphere of hydrogen (balloon) for 5 h. The mixture was diluted with ethyl acetate (ca. 50 mL), filtered through Celite and concentrated to give 0.070 g (87%) of the title compound as a grey white solid. APCI m/z 536 (M+H).

### Example 507

### N-{[3′-fluoro-3-({[(2,4,6-trimethylphenyl)amino]carbonyl}amino)-4-biphenylyl]carbonyl}-L-aspartic acid

A hot solution of LiOH (0.034 g, 1.40 mmol) in water (5 mL) was added to a solution of (2S)-4-(ethyloxy)-2-({[3′-fluoro-3-({[(2,4,6-trimethylphenyl)amino]carbonyl}amino)-4-biphenylyl]carbonyl}amino)-4-oxobutanoic acid (0.05 g, 0.09 mmol) in THF (5 mL) and MeOH (5 mL). After 3 h, the organic solvent was removed under reduced pressure then 1N HCl solution (5 mL) was added. The grey precipitate was filtered then dissolved in ethyl acetate (ca. 25 mL), dried over MgSO₄, filtered and concentrated to give 0.035 g (74%) of the title compound as a white solid. APCI m/z 508 (M+H).

### Example 508

### 1-({[3-({[(2,4,6-trimethylphenyl)amino]carbonyl}amino)-2-naphthalenyl]carbonyl}amino)cyclopentanecarboxylic acid

### Step 1

### Methyl 1-{[(3-amino-2-naphthalenyl)carbonyl]amino}cyclopentanecarboxylate

3-Amino-2-naphthoic acid (0.25 g, 1.11 mmol) and methyl 1-aminocyclopentanecarboxylate hydrochloride (0.22 g, 1.22 mmol) were dissolved in DMF (10 mL) and diisopropylethylamine (0.50 g, 3.9 mmol) and HATU (0.46 g, 1.22 mmol) were added. The solution was stirred for 2 h. The reaction was diluted with ethyl acetate and washed with water. The extracts were dried (MgSO₄) and concentrated onto SiO₂. Chromatography on SiO₂ eluting with ethyl acetate/hexanes provided 0.47 g of product as a yellow oil.

### Step 2

### Methyl 1-({[3-({[(2,4,6-trimethylphenyl)amino]carbonyl}amino)-2-naphthalenyl]carbonyl}amino)cyclopentanecarboxylate

Methyl 1-{[(3-amino-2-naphthalenyl)carbonyl]amino}cyclopentanecarboxylate (0.41 g, 1.31 mmol) was dissolved in pyridine (10 mL) and 2,4,6-trimethylphenyl isocyanate (0.53 g, 3.28 mmol) was added. The reaction was stirred for 3 h, diluted with ethyl acetate, and washed with water. The organics were dried (MgSO₄) and concentrated onto SiO2. Chromatography on SiO2 eluting with ethyl acetate provided 0.44 g of product as a orange solid.

### Step 3

### 1-({[3-({[(2,4,6-trimethylphenyl)amino]carbonyl}amino)-2-naphthalenyl]carbonyl}amino)cyclopentanecarboxylic acid

Methyl 1-({[3-({[(2,4,6-trimethylphenyl)amino]carbonyl}amino)-2-naphthalenyl]carbonyl}amino)cyclopentanecarboxylate (0.24 g, 0.50 mmol) was dissolved in 1:1 THF/MeOH (5 mL) and 2 M LiOH (2.5 mL) was added. The reaction was heated to 50° C. for 4 h and cooled. The solution was acidified with 1 M HCl (5 mL) and extracted with ethyl acetate. The extracts were dried (MgSO₄) and concentrated. MeOH was added to the residue and a solid formed. The solid was filtered off and the MeOH filtrate was purified by reverse-phase HPLC to afford 21 mg of product as a beige solid. ES MS m/z 460 (M+H).

### Example 509

### O-(Phenylmethyl)-N-{[3-({[(2,4,6-trimethylphenyl)amino]carbonyl}amino)-2-naphthalenyl]carbonyl}-L-serine

### Step 1

### N-[(3-Amino-2-naphthalenyl)carbonyl]-O-(phenylmethyl)-L-serine

3-Amino-2-naphthoic acid (0.29 g, 1.31 mmol) and O-(phenylmethyl)-L-serine hydrochloride (0.36 g, 1.45 mmol) were dissolved in DMF (10 mL) and diisopropylethylamine (0.60 g, 4.60 mmol) and HATU (0.55 g, 1.45 mmol) were added. The solution was stirred for 3 h, diluted with water, and extracted with ethyl acetate. The extracts were dried (MgSO₄) and concentrated onto SiO₂. Chromatography on SiO₂ eluting with ethyl acetate/hexanes provided 0.48 g of product as an orange solid.

### Step 2

### Methyl O-(phenylmethyl)-N-{[3-({[(2,4,6-trimethylphenyl)amino]carbonyl}amino)-2-naphthalenyl]carbonyl}-L-serinate

N-[(3-Amino-2-naphthalenyl)carbonyl]-O-(phenylmethyl)-L-serine (0.2 g, 0.55 mmol) was dissolved in pyridine (10 mL) and 2,4,6-trimethylphenylisocyanate (0.22 g, 1.37 mmol) was added. The reaction was stirred 3 h, diluted with ethyl acetate, and washed with 1 M HCl. The organic layer was dried (MgSO₄) and concentrated onto SiO₂. Chromatography on SiO₂ eluting with ethyl acetate/hexanes provided 0.13 g of product as a colorless solid.

### Step 3

### O-(Phenylmethyl)-N-{[3-({[(2,4,6-trimethylphenyl)amino]carbonyl}amino)-2-naphthalenyl]carbonyl}-L-serine

Methyl O-(phenylmethyl)-N-{[3-({[(2,4,6-trimethylphenyl)amino]carbonyl}amino)-2-naphthalenyl]carbonyl}-L-serinate (0.13 g, 0.24 mmol) was dissolved in 1:1 THF/MeOH (2 mL) and 2 M LiOH (1.2 mL) was added. The reaction was heated to 60° C. for 2 h, cooled, and acidified with 1 M HCl (0.5 mL). The solution was extracted with ethyl acetate and the extracts were dried (MgSO₄) and concentrated. The residue was dissolved in MeOH and purified by reverse-phase HPLC. The resulting solid was triturated with MeOH to afford 16 mg of product as a colorless solid. ES MS m/z 526 (M+H).

### Example 510

### N-{[3′,4′-Difluoro-3-({[(2,4,6-trimethylphenyl)amino]carbonyl}amino)-4-biphenylyl]carbonyl}-O-(phenylmethyl)-L-serine

### Step 1

### Methyl N-[(3′,4′-difluoro-3-nitro-4-biphenylyl)carbonyl]-O-(phenylmethyl)-L-serinate

3′,4′-Difluoro-3-nitro-4-biphenylcarboxylic acid (0.2 g, 0.72 mmol) and O-(phenylmethyl)-L-serine hydrochloride (0.19 g, 0.78 mmol) were dissolved in DMF (5 mL) and diisopropylethylamine (0.32 g, 2.50 mmol) and HATU (0.30 g, 0.78 mmol) were added. The solution was stirred overnight, diluted with ethyl acetate, and washed with water. The organic layer was dried (MgSO₄) and concentrated. Chromatography on SiO₂ eluting with ethyl acetate/hexanes provided 0.3 g of product as a colorless solid.

### Step 2

### Methyl N-[(3-amino-3′,4′-difluoro-4-biphenylyl)carbonyl]-O-(phenylmethyl)-L-serinate

Methyl N-[(3′,4′-difluoro-3-nitro-4-biphenylyl)carbonyl]-O-(phenylmethyl)-L-serinate (0.3 g, 0.63 mmol) was dissolved in EtOH (7 mL) and saturated NH₄Cl (3 mL) and indium powder (0.6 g) was added. The reaction was heated to reflux for 5 h, cooled, diluted with water and extracted with ethyl acetate. The extracts were dried (MgSO₄) and concentrated. Chromatography on SiO₂ eluting with ethyl acetate/hexanes provided 0.19 g of product.

### Step 3

### Methyl N-{[3′,4′-difluoro-3-({[(2,4,6-trimethylphenyl)amino]carbonyl}amino)-4-biphenylyl]carbonyl}-O-(phenylmethyl)-L-serinate

Methyl N-[(3-amino-3′,4′-difluoro-4-biphenylyl)carbonyl]-O-(phenylmethyl)-L-serinate (0.19 g, 0.44 mmol) was dissolved in pyridine (5 mL) and 2,4,6-trimethylphenylisocyanate (0.25 g, 1.54 mmol) was added. The reaction was stirred for 4 h, diluted with ethyl acetate, and washed with 1 M HCl. The organics were dried (MgSO₄) and concentrated onto SiO₂. Chromatography on SiO₂ eluting with ethyl acetate/hexanes provided 0.19 g of product.

### Step 4

### N-{[3′,4′-Difluoro-3-({[(2,4,6-trimethylphenyl)amino]carbonyl}amino)-4-biphenylyl]carbonyl}-O-(phenylmethyl)-L-serine

Methyl N-{[3′,4′-difluoro-3-({[(2,4,6-trimethylphenyl)amino]carbonyl}amino)-4-biphenylyl]carbonyl}-O-(phenylmethyl)-L-serinate (0.19 g, 0.31 mmol) was dissolved in 1:1 THF/MeOH (5 mL) and 2 M LiOH (1.6 mL) was added. The reaction was stirred overnight, acidified with 1 M HCl (3.2 mL) and extracted with ethyl acetate. The extracts were dried (MgSO₄) and concentrated. The residue was dissolved in MeOH and a solid formed. The solid was collected to afford 24 mg of product as a colorless solid. ES MS m/z 588 (M+H).

### Example 511

### (3R)-5-Methyl-3-[(phenylmethyl)oxy]-N-{[3-({[(2,4,6-trimethylphenyl)amino]carbonyl}amino)-2-naphthalenyl]carbonyl}-L-norleucine

### Step 1

### (1R)-3-Methyl-1-[(2S,5R)-5-(1-methylethyl)-3,6-bis(methyloxy)-2,5-dihydro-2-pyrazinyl]-1-butanol

(2R)-2-(1-methylethyl)-3,6-bis(methyloxy)-2,5-dihydropyrazine (1 g, 5.42 mmol) was dissolved in THF (40 mL) and cooled to −78° C. A solution of n-BuLi (3.8 mL of a 1.6 M solution) was added dropwise and stirred for 30 min. 3-Methylbutanal (0.51 mL, 5.97 mmol) was added and the reaction was stirred overnight. The mixture was poured onto water and extracted with ethyl acetate. The extracts were separated, dried (MgSO₄), and concentrated onto SiO₂. Chromatography on SiO₂ eluting with ethyl acetate/hexanes afforded 1.13 g of product as a clear oil.

### Step 2

### (2R,5S)-2-(1-Methylethyl)-3,6-bis(methyloxy)-5-{(1R)-3-methyl-1-[(phenylmethyl)oxy]butyl}-2,5-dihydropyrazine

(1R)-3-Methyl-1-[(2S,5R)-5-(1-methylethyl)-3,6-bis(methyloxy)-2,5-dihydro-2-pyrazinyl]-1-butanol (1.13 g, 4.18 mmol) was dissolved in DMF (20 mL) and the solution was cooled to 0° C. Sodium hydride (0.20 g, 5.0 mmol) was added and stirred 20 min and then benzyl bromide (0.79 g, 4.59 mmol) was added and stirred 3 days. The reaction was diluted with ethyl acetate, washed with water, and the organics were dried (MgSO₄) and concentrated onto SiO₂. Chromatography on SiO₂ eluting with ethyl acetate/hexanes afforded 1.05 g of product as a colorless oil.

### Step 3

### Methyl(3R)-5-methyl-3-[(phenylmethyl)oxy]-L-norleucinate

(2R,5S)-2-(1-Methylethyl)-3,6-bis(methyloxy)-5-{(1R)-3-methyl-1-[(phenylmethyl)oxy]butyl}-2,5-dihydropyrazine (1.05 g, 2.91 mmol) was dissolved in CH₃CN (12 mL) and 0.5 N HCl (11.6 mL) was added and the solution was stirred for 2 days. Sodium chloride and Et₂O were added to the solution and the pH was adjusted to 9 with ammonium hydroxide. The mixture was extracted with Et₂O, the extracts were combined and concentrated to afford 0.33 g of oil as a 1:1 mixture of desired product and methyl D-valinate.

### Step 4

### Methyl(3R)—N-[(3-amino-2-naphthalenyl)carbonyl]-5-methyl-3-[(phenylmethyl)oxy]-L-norleucinate

A 1:1 mixture of Methyl(3R)-5-methyl-3-[(phenylmethyl)oxy]-L-norleucinate and methyl D-valinate (0.33 g, 0.83 mmol) and 3-amino-2-naphthalenecarboxylic acid (0.22 g, 1.0 mmol) was dissolved in DMF (5 mL) and diisopropylethylamine (0.32 g, 2.50 mmol) was added followed by HATU (0.38 g, 1.0 mmol). The solution was stirred overnight and then diluted with water and extracted with ethyl acetate. The extracts were dried (MgSO₄), concentrated onto SiO₂, and purified by chromatography on SiO₂ eluting with ethyl acetate/hexanes to afford 0.21 g of a 1:1 mixture of product and methyl N-[(3-amino-2-naphthalenyl)carbonyl]-D-valinate.

### Step 5

### Methyl(3R)-5-methyl-3-[(phenylmethyl)oxy]-N-{[3-({[(2,4,6-trimethylphenyl)amino]carbonyl}amino)-2-naphthalenyl]carbonyl}-L-norleucinate

A 1:1 mixture of methyl(3R)—N-[(3-amino-2-naphthalenyl)carbonyl]-5-methyl-3-[(phenylmethyl)oxy]-L-norleucinate and methyl N-[(3-amino-2-naphthalenyl)carbonyl]-D-valinate (0.21 g, 0.28 mmol) was dissolved in pyridine (5 mL) and 2,4,6-trimethylphenylisocyanate (0.27 g, 1.71 mmol) was added and stirred for 3 h. The solution was diluted with MeOH and filtered. The filtrate was concentrated and the resulting residue was dissolved in ethyl acetate, washed with 1 M HCl, and dried (MgSO₄). The solution was concentrated to afford an orange oil weighing 0.50 g, consisting of a 1:1 mixture of product and methyl N-{[3-({[(2,4,6-trimethylphenyl)amino]carbonyl}amino)-2-naphthalenyl]carbonyl}-D-valinate.

### Step 6

### (3R)-5-Methyl-3-[(phenylmethyl)oxy]-N-{[3-({[(2,4,6-trimethylphenyl)amino]carbonyl}amino)-2-naphthalenyl]carbonyl}-L-norleucine

A 1:1 mixture of methyl(3R)-5-methyl-3-[(phenylmethyl)oxy]-N-{[3-({[(2,4,6-trimethylphenyl)amino]carbonyl}amino)-2-naphthalenyl]carbonyl}-L-norleucinate and methyl N-{[3-({[(2,4,6-trimethylphenyl)amino]carbonyl}amino)-2-naphthalenyl]carbonyl}-D-valinate (0.50 g, 0.28 mmol) was dissolved in 1:1 THF/MeOH (5 mL) and 2 M LiOH (1.4 mL) was added. The reaction was stirred for 3 h, acidified with 1 M HCl (2.8 mL) and extracted with ethyl acetate. The extracts were dried (MgSO₄) and concentrated. A 200 mg sample of the residue was dissolved in MeOH (1 mL) and purified by reverse-phase HPLC to afford 46 mg of product as a brown solid. MS m/z 582 (M+H).

### Example 512

### O-cyclobutyl-N-{[3-({[(2,4,6-trimethylphenyl)amino]carbonyl}amino)-2-naphthalenyl]carbonyl}-L-threonine

### Step 1

### Methyl N-(triphenylmethyl)-L-threoninate

To a cooled (0° C.) solution of methyl L-threoninate hydrochloride (5.0 g, 29.48 mmol) and triethylamine (5.97 g, 58.97 mmol) in chloroform (100 ml) was added trityl chloride as a solid (8.22 g, 29.49 mmol). The reaction was stirred for 12 hours and allowed to come to RT. The reaction was concentrated in vacuo and then dissolved in ethyl acetate and washed with saturated sodium chloride, 10% citric acid, saturated NaHCO₃, and saturated sodium chloride. The organic layer was dried over MgSO₄, filtered and stripped to give 10.16 g of product as a fluffy cream solid. ES MS m/z 398 (M+Na).

### Step 2

### Methyl(2R,3S)-3-methyl-1-(triphenylmethyl)-2-aziridinecarboxylate

To a cooled (0° C.) solution of methyl N-(triphenylmethyl)-L-threoninate (10.16 g, 27.95 mmol) in anhydrous pyridine was added methanesulfonyl chloride (9.61 g, 83.85 mmol) and the reaction was allowed to stir for 12 hours and allowed to come to RT. The solvent was removed in vacuo and the residue dissolved in ethyl acetate. The organic layer was washed with saturated sodium chloride and then dried over MgSO₄, filtered and stripped to 12.33 g of amber oil which was then dissolved in 80 ml of anhydrous THF and to which was added triethylamine (8.50 g, 84.01 mmol) and heated to 80° C. and allowed to reflux for 48 hours. The heat was removed and the reaction was concentrated in vacuo and the residue dissolved in ethyl acetate and washed successively with saturated sodium chloride, 10% citric acid, saturated NaHCO₃ and saturated sodium chloride. The ethyl acetate layer was dried over MgSO₄, filtered and stripped to give 9.04 g of amber oil. Chromatography on silica gel with hexane/ethyl acetate gave 5.26 g of product fluffy cream solid. ES MS m/z 380 (M+Na).

### Step 3

### 2-methyl 1-(phenylmethyl) (2R,3S)-3-methyl-1,2-aziridinedicarboxylate

To a solution of methyl(2R,3S)-3-methyl-1-(triphenylmethyl)-2-aziridinecarboxylate (5.26 g, 14.72 mmol) in CHCl₃ (12 ml) and MeOH (12 ml) cooled to 0° C. was added 11.6 ml of TFA and allowed to stir at 0° C. for 2.5 hours. The reaction was then concentrated in vacuo and evaporated with ether newly added several times to remove TFA. The residue was dissolved in ether which was extracted with water three times. To the aqueous extract at 0° C. was added NaHCO₃ (5.84 g, 69.52 mmol), benzyl chloroformate (2.51 g, 14.71 mmol) and 50 ml of ethyl acetate under vigorous stirring for 1.5 hours. The ethyl acetate layer was separated and the water layer back-extracted. The organics were dried over MgSO₄, filtered and concentrated to give 2.96 g of light yellow oil. Chromatography on silica gel with hexane/ethyl acetate gave 2.45 g of product as a clear oil. ES MS m/z 250 (M+H).

### Step 4

### Methyl O-cyclobutyl-N-{[(phenylmethyl)oxy]carbonyl}-L-threoninate

To a solution of 2-methyl 1-(phenylmethyl) (2R,3S)-3-methyl-1,2-aziridinedicarboxylate (0.4 g, 1.60 mmol) in CHCl₃ (10 ml) was added cyclobutanol (1.16 g, 16.09 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 0.76 g of product as a rose oil.

### Step 5

### Methyl O-cyclobutyl-L-threoninate

Palladium (10% weight on activated carbon, catalytic amount) was added to a solution of methyl O-cyclobutyl-N-{[(phenylmethyl)oxy]carbonyl}-L-threoninate (0.76 g, 2.36 mmol) in 10 ml of EtOH in a flask under nitrogen. A balloon of H₂ was then affixed to the reaction flask and the reaction was stirred for 2 hours at RT. The reaction was then filtered through a filter paper and the solvent evaporated to give 0.37 g of clear oil.

### Step 6

### Methyl N-[(3-amino-2-naphthalenyl)carbonyl]-O-cyclobutyl-L-threoninate

HATU (0.76 g, 2.00 mmol) was added to a solution of 3-amino-2-naphthalenecarboxylic acid (0.31 g, 1.66 mmol), methyl O-cyclobutyl-L-threoninate (0.37 g, 1.98 mmol) and diisopropylethylamine (0.26 g, 2.01 mmol) in 15 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.27 g of yellow oil.

### Step 7

### Methyl O-cyclobutyl-N-{[3-({[(2,4,6-trimethylphenyl)amino]carbonyl}amino)-2-naphthalenyl]carbonyl}-L-threoninate

Methyl N-[(3-amino-2-naphthalenyl)carbonyl]-O-cyclobutyl-L-threoninate (0.27 g, 0.76 mmol) in 20 mL of pyridine was treated with 2-isocyanato-1,3,5-trimethylbenzene (0.61 g, 3.77 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.22 g of product as cream solid.

### Step 8

### O-cyclobutyl-N-{[3-({[(2,4,6-trimethylphenyl)amino]carbonyl}amino)-2-naphthalenyl]carbonyl}-L-threonine

Lithium hydroxide monohydrate (0.102 g, 4.26 mmol) was added to a solution of methyl O-cyclobutyl-N-{[3-({[(2,4,6-trimethylphenyl)amino]carbonyl}amino)-2-naphthalenyl]carbonyl}-L-threoninate (0.22 g, 0.425 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.066 g (30% yield) of product as a fluffy cream solid. ES MS m/z 504 (M+H).

### Example 513

### 1-{[(3-{[(4-biphenylylamino)carbonyl]amino}-2-naphthalenyl)carbonyl]amino}cyclohexanecarboxylic acid

### Step 1

### 1-{[(3-{[(4-biphenylylamino)carbonyl]amino}-2-naphthalenyl)carbonyl]amino}cyclohexanecarboxylic acid

1-{[(3-amino-2-naphthalenyl)carbonyl]amino}cyclohexanecarboxylic acid (0.04 g, 0.13 mmol) in 5 mL of pyridine was treated with 4-isocyanatobiphenyl (0.12 g, 0.61 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.038 g of product as cream solid. ES MS m/z 508 (M+H).

### Example 514

### N-{[3-({[(2,4,6-trimethylphenyl)amino]carbonyl}amino)-2-naphthalenyl]carbonyl}-L-phenylalanine

### Step 1

### Ethyl N-[(3-amino-2-naphthalenyl)carbonyl]-L-phenylalaninate

HATU (1.22 g, 3.21 mmol) was added to a solution of 3-amino-2-naphthalenecarboxylic acid (0.5 g, 2.67 mmol), ethyl L-phenylalaninate hydrochloride (0.74 g, 3.22 mmol) and diisopropylethylamine (0.41 g, 3.21 mmol) in 15 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.79 g of yellow oil.

### Step 2

### Ethyl N-{[3-({[(2,4,6-trimethylphenyl)amino]carbonyl}amino)-2-naphthalenyl]carbonyl}-L-phenylalaninate

Ethyl N-[(3-amino-2-naphthalenyl)carbonyl]-L-phenylalaninate (0.79 g, 2.18 mmol) in 10 mL of pyridine was treated with 2-isocyanato-1,3,5-trimethylbenzene (1.76 g, 10.89 mmol) for ca. 15 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.47 g of product as light pink semi-solid.

### Step 3

### N-{[3-({[(2,4,6-trimethylphenyl)amino]carbonyl}amino)-2-naphthalenyl]carbonyl}-L-phenylalanine

Lithium hydroxide monohydrate (0.215 g, 8.98 mmol) was added to a solution of ethyl N-{[3-({[(2,4,6-trimethylphenyl)amino]carbonyl}amino)-2-naphthalenyl]carbonyl}-L-phenylalaninate (0.47 g, 0.90 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.072 g (16% yield) of product as a white solid. ES MS m/z 496 (M+H).

### Example 515

### (2S)-4-({[(1,1-dimethylethyl)oxy]carbonyl}amino)-2-({[4-fluoro-2-({[(2,4,6-trimethylphenyl)amino]carbonyl}amino)phenyl]carbonyl}amino)butanoic acid

### Step 1

### Methyl(2S)-2-{[(2-amino-4-fluorophenyl)carbonyl]amino}-4-({[(1,1-dimethylethyl)oxy]carbonyl}amino)butanoate

HATU (1.48 g, 3.89 mmol) was added to a solution of 2-amino-4-fluorobenzoic acid (0.5 g, 3.22 mmol), methyl(2S)-2-amino-4-({[(1,1-dimethylethyl)oxy]carbonyl}amino)butanoate hydrochloride (1.04 g, 3.87 mmol) and diisopropylethylamine (0.50 g, 3.90 mmol) in 25 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated to give 1.6 g of fluffy cream solid.

### Step 2

### Methyl(2S)-4-({[(1,1-dimethylethyl)oxy]carbonyl}amino)-2-({[4-fluoro-2-({[(2,4,6-trimethylphenyl)amino]carbonyl}amino)phenyl]carbonyl}amino)butanoate

Methyl(2S)-2-{[(2-amino-4-fluorophenyl)carbonyl]amino}-4-({[(1,1-dimethylethyl)oxy]carbonyl}amino)butanoate (0.62 g, 1.68 mmol) in 20 mL of pyridine was treated with 2-isocyanato-1,3,5-trimethylbenzene (1.36 g, 8.42 mmol) for ca. 15 h at RT. The reaction was quenched with 1N HCl and extracted with ethyl acetate. The organic layer dried over magnesium sulfate, filtered, and the solvent evaporated to give 1.39 g of product as white semi-solid.

### Step 3

### (2S)-4-({[(1,1-dimethylethyl)oxy]carbonyl}amino)-2-({[4-fluoro-2-({[(2,4,6-trimethylphenyl)amino]carbonyl}amino)phenyl]carbonyl}amino)butanoic acid

Lithium hydroxide monohydrate (0.27 g, 11.27 mmol) was added to a solution of methyl(2S)-4-({[(1,1-dimethylethyl)oxy]carbonyl}amino)-2-({[4-fluoro-2-({[(2,4,6-trimethylphenyl)amino]carbonyl}amino)phenyl]carbonyl}amino)butanoate (0.6 g, 1.13 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.077 g (11% yield) of product as a light orange fluffy solid. ES MS m/z 517 (M+H).

### Example 516

### (2S)-4-({[(1,1-dimethylethyl)oxy]carbonyl}amino)-2-({[3-({[(2,4,6-trimethylphenyl)amino]carbonyl}amino)-2-naphthalenyl]carbonyl}amino)butanoic acid

### Step 1

### Methyl(2S)-2-{[(3-amino-2-naphthalenyl)carbonyl]amino}-4-({[(1,1-dimethylethyl)oxy]carbonyl}amino)butanoate

HATU (1.22 g, 3.21 mmol) was added to a solution of 3-amino-2-naphthalenecarboxylic acid (0.5 g, 2.67 mmol), methyl(2S)-2-amino-4-({[(1,1-dimethylethyl)oxy]carbonyl}amino)butanoate hydrochloride (0.86 g, 3.20 mmol) and diisopropylethylamine (0.41 g, 3.21 mmol) in 20 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.86 g of yellow oil.

### Step 2

### Methyl(2S)-4-({[(1,1-dimethylethyl)oxy]carbonyl}amino)-2-({[3-({[(2,4,6-trimethylphenyl)amino]carbonyl}amino)-2-naphthalenyl]carbonyl}amino)butanoate

Methyl(2S)-2-{[(3-amino-2-naphthalenyl)carbonyl]amino}-4-({[(1,1-dimethylethyl)oxy]carbonyl}amino)butanoate (0.3 g, 0.75 mmol) in 10 mL of pyridine was treated with 2-isocyanato-1,3,5-trimethylbenzene (0.6 g, 3.71 mmol) for ca. 3 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.15 g of product as a cream solid.

### Step 3

### (2S)-4-({[(1,1-dimethylethyl)oxy]carbonyl}amino)-2-({[3-({[(2,4,6-trimethylphenyl)amino]carbonyl}amino)-2-naphthalenyl]carbonyl}amino)butanoic acid

Lithium hydroxide monohydrate (0.06 g, 2.50 mmol) was added to a solution of methyl(2S)-4-({[(1,1-dimethylethyl)oxy]carbonyl}amino)-2-({[3-({[(2,4,6-trimethylphenyl)amino]carbonyl}amino)-2-naphthalenyl]carbonyl}amino)butanoate (0.15 g, 0.27 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.044 g (30% yield) of product as a fluffy white solid. ES MS m/z 549 (M+H).

### Example 517

### 5,5-dimethyl-N-{[3-({[(2,4,6-trimethylphenyl)amino]carbonyl}amino)-2-naphthalenyl]carbonyl}norleucine

### Step 1: Methyl(2E)-5,5-dimethyl-2-({[(phenylmethyl)oxy]carbonyl}amino)-2-hexenoate

To a solution of methyl[bis(methyloxy)phosphoryl]({[(phenylmethyl)oxy]carbonyl}amino)acetate (1.82 g, 5.49 mmol) in CH₂Cl₂ was added DBU (0.84 g, 5.52 mmol) and the resulting solution was stirred at RT for 10 minutes. To that was then added 3,3-dimethylbutanal (0.5 g, 4.99 mmol), and the reaction was stirred for 16 hours at RT. The reaction was quenched with 1N HCl and the CH₂Cl₂ layer dried over sodium sulfate, filtered, and the solvent evaporated. Chromatography on silica gel with hexane/ethyl acetate gave 1.6 g of product as clear oil.

### Step 2

### Methyl 5,5-dimethylnorleucinate

Palladium (10% weight on activated carbon, catalytic amount) was added to a solution of methyl(2E)-5,5-dimethyl-2-({[(phenylmethyl)oxy]carbonyl}amino)-2-hexenoate (1.6 g, 5.24 mmol) in 25 ml of EtOH in a flask under nitrogen. A balloon of H₂ was then affixed to the reaction flask and the reaction was stirred for 16 hours at RT. The reaction was then filtered through a filter paper and the solvent evaporated to give 0.50 g of clear oil.

### Step 3

### Methyl N-[(3-amino-2-naphthalenyl)carbonyl]-5,5-dimethyinorleucinate

HATU (1.10 g, 2.89 mmol) was added to a solution of 3-amino-2-naphthalenecarboxylic acid (0.45 g, 2.40 mmol), methyl 5,5-dimethyinorleucinate (0.5 g, 2.89 mmol) and diisopropylethylamine (0.38 g, 2.93 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated Chromatography on silica gel with hexane/ethyl acetate gave 0.74 g of yellow oil.

### Step 4

### Methyl 5,5-dimethyl-N-{[3-({[(2,4,6-trimethylphenyl)amino]carbonyl}amino)-2-naphthalenyl]carbonyl}norleucinate

Methyl N-[(3-amino-2-naphthalenyl)carbonyl]-5,5-dimethylnorleucinate (0.74 g, 2.16 mmol) in 10 mL of pyridine was treated with 2-isocyanato-1,3,5-trimethylbenzene (1.75 g, 10.83 mmol) for ca. 3 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.69 g of product as a cream solid.

### Step 5

### 5,5-dimethyl-N-{[3-({[(2,4,6-trimethylphenyl)amino]carbonyl}amino)-2-naphthalenyl]carbonyl}norleucine

Lithium hydroxide monohydrate (0.33 g, 13.78 mmol) was added to a solution of methyl 5,5-dimethyl-N-{[3-({[(2,4,6-trimethylphenyl)amino]carbonyl}amino)-2-naphthalenyl]carbonyl}norleucinate (0.69 g, 1.37 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.44 g (65% yield) of product as a fluffy amber solid. ES MS m/z 489 (M+H).

### Example 518

### 1-({[3-({[(3,5-dimethyl-4-biphenylyl)amino]carbonyl}amino)-2-naphthalenyl]carbonyl}amino)cycloheptanecarboxylic acid

### Step 1

### Methyl 1-({[3-({[(3,5-dimethyl-4-biphenylyl)amino]carbonyl}amino)-2-naphthalenyl]carbonyl}amino)cycloheptanecarboxylate

To a solution of methyl 1-({[3-({[(4-bromo-2,6-dimethylphenyl)amino]carbonyl}amino)-2-naphthalenyl]carbonyl}amino)cycloheptanecarboxylate (0.17 g, 0.30 mmol) in DME (5 ml) was added tetrakis(triphenylmethyl)palladium (0.01 g, 0.008 mmol), phenylboronic acid (0.055 g, 0.45 mmol) and 2M Na2CO3 (0.3 ml). The reaction was heated to 110° C. for 16 hours and then loaded directly onto silica. Chromatography on silica gel with hexane/ethyl acetate gave 0.36 g of product as yellow semi-solid.

### Step 2

### 1-({[3-({[(3,5-dimethyl-4-biphenylyl)amino]carbonyl}amino)-2-naphthalenyl]carbonyl}amino)cycloheptanecarboxylic acid

Lithium hydroxide monohydrate (0.13 g, 5.43 mmol) was added to a solution of methyl 1-({[3-({[(3,5-dimethyl-4-biphenylyl)amino]carbonyl}amino)-2-naphthalenyl]carbonyl}amino)cycloheptanecarboxylate (0.31 g, 0.55 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.024 g (8% yield) of product as a cream solid. ES MS m/z 550 (M+H).

### Example 519

### O-cyclobutyl-N-{[3′,4′-difluoro-3-({[(2,4,6-trimethylphenyl)amino]carbonyl}amino)-4-biphenylyl]carbonyl}-L-threonine

### Step 1

### Methyl N-(triphenylmethyl)-L-threoninate

To a cooled (0° C.) solution of methyl L-threoninate hydrochloride (4.0 g, 23.58 mmol) and triethylamine (4.78 g, 47.21 mmol) in chloroform (100 ml) was added trityl chloride as a solid (6.57 g, 23.57 mmol). The reaction was stirred for 12 hours and allowed to come to RT. The reaction was concentrated in vacuo and then dissolved in ethyl acetate and washed with saturated sodium chloride, 10% citric acid, saturated NaHCO₃, and saturated sodium chloride. The organic layer was dried over MgSO₄, filtered and stripped to give 9.14 g of product as an amber oil.

### Step 2

### Methyl(2R,3S)-3-methyl-1-(triphenylmethyl)-2-aziridinecarboxylate

To a cooled (0° C.) solution of methyl N-(triphenylmethyl)-L-threoninate (9.14 g, 25.15 mmol) in anhydrous pyridine was added methanesulfonyl chloride (8.64 g, 75.45 mmol) and the reaction was allowed to stir for 12 hours and allowed to come to RT. The solvent was removed in vacuo and the residue dissolved in ethyl acetate. The organic layer was washed with saturated sodium chloride and then dried over MgSO₄, filtered and stripped to a brown oil which was then dissolved in 80 ml of anhydrous THF and to which was added triethylamine (7.59 g, 74.97 mmol) and heated to 80° C. and allowed to reflux for 16 hours.

The heat was removed and the reaction was concentrated in vacuo and the residue dissolved in ethyl acetate and washed successively with saturated sodium chloride, 10% citric acid, saturated NaHCO₃ and saturated sodium chloride. The ethyl acetate layer was dried over MgSO₄, filtered and stripped. Chromatography on silica gel with hexane/ethyl acetate gave 4.6 g of product as a yellow oil.

### Step 3

### 2-methyl 1-(phenylmethyl) (2R,3S)-3-methyl-1,2-aziridinedicarboxylate

To a solution of methyl(2R,3S)-3-methyl-1-(triphenylmethyl)-2-aziridinecarboxylate (4.6 g, 12.87 mmol) in CHCl₃ (12 ml) and MeOH (12 ml) cooled to 0° C. was added 11.6 ml of TFA and allowed to stir at 0° C. for 2.5 hours. The reaction was then concentrated in vacuo and evaporated with ether newly added several times to remove TFA. The residue was dissolved in ether which was extracted with water three times. To the aqueous extract at 0° C. was added NaHCO₃ (5.12 g, 60.95 mmol), benzyl chloroformate (2.21 g, 12.96 mmol) and 50 ml of ethyl acetate under vigorous stirring for 1.5 hours. The ethyl acetate layer was separated and the water layer back-extracted. The organics were dried over MgSO₄, filtered and concentrated to give 3.45 g of light yellow oil. Chromatography on silica gel with hexane/ethyl acetate gave 2.22 g of product as a clear oil.

### Step 4

### Methyl O-cyclobutyl-N-{[(phenylmethyl)oxy]carbonyl}-L-threoninate

To a solution of 2-methyl 1-(phenylmethyl) (2R,3S)-3-methyl-1,2-aziridinedicarboxylate (0.58 g, 2.33 mmol) in CHCl₃ (10 ml) was added cyclobutanol (1.68 g, 23.25 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 0.89 g of product as clear oil.

### Step 5

### Methyl O-cyclobutyl-L-threoninate

Palladium (10% weight on activated carbon, catalytic amount) was added to a solution of methyl O-cyclobutyl-N-{[(phenylmethyl)oxy]carbonyl}-L-threoninate (0.89 g, 2.77 mmol) in 10 ml of EtOH in a flask under nitrogen. A balloon of H₂ was then affixed to the reaction flask and the reaction was stirred for 2 hours at RT. The reaction was then filtered through a filter paper and the solvent evaporated to give 0.33 g of clear oil.

### Step 6

### Methyl O-cyclobutyl-N-[(3′,4′-difluoro-3-nitro-4-biphenylyl)carbonyl]-L-threoninate

HATU (0.67 g, 1.76 mmol) was added to a solution of 3-amino-3′,4′-difluoro-4-biphenylcarboxylic acid (0.41 g, 1.47 mmol), methyl O-cyclobutyl-L-threoninate (0.33 g, 1.76 mmol) and diisopropylethylamine (0.23 g, 1.78 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 1.09 g of yellow oil.

### Step 7

### Methyl N-[(3-amino-3′,4′-difluoro-4-biphenylyl)carbonyl]-O-cyclobutyl-L-threoninate

To a solution of methyl O-cyclobutyl-N-[(3′,4′-difluoro-3-nitro-4-biphenylyl)carbonyl]-L-threoninate (1.90 g, 2.43 mmol) in 25 ml of ethanol was added 11 ml of saturated ammonium chloride and indium (2.18 g, 18.99 mmol). The reaction was heated to reflux for 16 hours and then diluted with water and ethyl acetate. The organic layer was dried over magnesium sulfate filtered and concentrated. Chromatography on silica gel with hexane/ethyl acetate gave 0.32 g of yellow residue.

### Step 8

### Methyl O-cyclobutyl-N-{[3′,4′-difluoro-3-({[(2,4,6-trimethylphenyl)amino]carbonyl}amino)-4-biphenylyl]carbonyl}-L-threoninate

Methyl N-[(3-amino-3′,4′-difluoro-4-biphenylyl)carbonyl]-O-cyclobutyl-L-threoninate (0.32 g, 0.76 mmol) in 10 mL of pyridine was treated with 2-isocyanato-1,3,5-trimethylbenzene (0.62 g, 3.84 mmol) for ca. 16 h at RT. The reaction was quenched with 1N HCl and extracted with ethyl acetate. The organic layer dried over magnesium sulfate, filtered, and the solvent evaporated to give 0.35 g of product as a light yellow solid.

### Step 9

### O-cyclobutyl-N-{[3′,4′-difluoro-3-({[(2,4,6-trimethylphenyl)amino]carbonyl}amino)-4-biphenylyl]carbonyl}-L-threonine

Lithium hydroxide monohydrate (0.14 g, 5.85 mmol) was added to a solution of methyl O-cyclobutyl-N-{[3′,4′-difluoro-3-({[(2,4,6-trimethylphenyl)amino]carbonyl}amino)-4-biphenylyl]carbonyl}-L-threoninate (0.35 g, 0.60 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.200 g (61% yield) of product as a fluffy orange solid. ES MS m/z 566 (M+H).

### Example 520

### O-(1-methylcyclopentyl)-N-{[3-({[(2,4,6-trimethylphenyl)amino]carbonyl}amino)-2-naphthalenyl]carbonyl}-L-threonine

### Step 1

### Methyl N-(triphenylmethyl)-L-threoninate

To a cooled (0° C.) solution of methyl L-threoninate hydrochloride (4.0 g, 23.58 mmol) and triethylamine (4.78 g, 47.21 mmol) in chloroform (100 ml) was added trityl chloride as a solid (6.57 g, 23.57 mmol). The reaction was stirred for 12 hours and allowed to come to RT. The reaction was concentrated in vacuo and then dissolved in ethyl acetate and washed with saturated sodium chloride, 10% citric acid, saturated NaHCO₃, and saturated sodium chloride. The organic layer was dried over MgSO₄, filtered and stripped to give 9.14 g of product as an amber oil.

### Step 2

### Methyl(2R,3S)-3-methyl-1-(triphenylmethyl)-2-aziridinecarboxylate

To a cooled (0° C.) solution of methyl N-(triphenylmethyl)-L-threoninate (9.14 g, 25.15 mmol) in anhydrous pyridine was added methanesulfonyl chloride (8.64 g, 75.45 mmol) and the reaction was allowed to stir for 12 hours and allowed to come to RT. The solvent was removed in vacuo and the residue dissolved in ethyl acetate. The organic layer was washed with saturated sodium chloride and then dried over MgSO₄, filtered and stripped to a brown oil which was then dissolved in 80 ml of anhydrous THF and to which was added triethylamine (7.59 g, 74.97 mmol) and heated to 80° C. and allowed to reflux for 16 hours.

The heat was removed and the reaction was concentrated in vacuo and the residue dissolved in ethyl acetate and washed successively with saturated sodium chloride, 10% citric acid, saturated NaHCO₃ and saturated sodium chloride. The ethyl acetate layer was dried over MgSO₄, filtered and stripped. Chromatography on silica gel with hexane/ethyl acetate gave 4.6 g of product as a yellow oil.

### Step 3

### 2-methyl 1-(phenylmethyl) (2R,3S)-3-methyl-1,2-aziridinedicarboxylate

To a solution of methyl(2R,3S)-3-methyl-1-(triphenylmethyl)-2-aziridinecarboxylate (4.6 g, 12.87 mmol) in CHCl₃ (12 ml) and MeOH (12 ml) cooled to 0° C. was added 11.6 ml of TFA and allowed to stir at 0° C. for 2.5 hours. The reaction was then concentrated in vacuo and evaporated with ether newly added several times to remove TFA. The residue was dissolved in ether which was extracted with water three times. To the aqueous extract at 0° C. was added NaHCO₃ (5.12 g, 60.95 mmol), benzyl chloroformate (2.21 g, 12.96 mmol) and 50 ml of ethyl acetate under vigorous stirring for 1.5 hours. The ethyl acetate layer was separated and the water layer back-extracted. The organics were dried over MgSO₄, filtered and concentrated to give 3.45 g of light yellow oil. Chromatography on silica gel with hexane/ethyl acetate gave 2.22 g of product as a clear oil.

### Step 4

### Methyl O-(1-methylcyclopentyl)-N-{[(phenylmethyl)oxy]carbonyl}-L-threoninate

To a solution of 2-methyl 1-(phenylmethyl) (2R,3S)-3-methyl-1,2-aziridinedicarboxylate (1.00 g, 4.01 mmol) in CHCl₃ (10 ml) was added cyclobutanol (4.02 g, 40.14 mmol) and boron trifluoride diethyl etherate (5 drops) and stirred for 16 hours. The reaction was quenched with H₂O and extracted with CH₂Cl₂. The CH₂Cl₂ layer was dried over magnesium sulfate, filtered and concentrated in vacuo to give 1.21 g of product as grey oil.

### Step 5

### Methyl O-(1-methylcyclopentyl)-L-threoninate

Palladium (10% weight on activated carbon, catalytic amount) was added to a solution of methyl O-cyclobutyl-N-{[(phenylmethyl)oxy]carbonyl}-L-threoninate (1.21 g, 3.46 mmol) in 10 ml of EtOH in a flask under nitrogen. A balloon of H₂ was then affixed to the reaction flask and the reaction was stirred for 2 hours at RT. The reaction was then filtered through a filter paper and the solvent evaporated to give 0.64 g of tan oil.

### Step 6

### Methyl N-[(3-amino-2-naphthalenyl)carbonyl]-O-(1-methylcyclopentyl)-L-threoninate

HATU (1.13 g, 2.97 mmol) was added to a solution of 3-amino-2-naphthalenecarboxylic acid (0.46 g, 2.46 mmol), methyl O-(1-methylcyclopentyl)-L-threoninate (0.64 g, 2.97 mmol) and diisopropylethylamine (0.38 g, 2.98 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated. Chromatography on silica gel with hexane/ethyl acetate gave 0.30 g of yellow oil.

### Step 7

### Methyl O-(1-methylcyclopentyl)-N-{[3-({[(2,4,6-trimethylphenyl)amino]carbonyl}amino)-2-naphthalenyl]carbonyl}-L-threoninate

Methyl N-[(3-amino-2-naphthalenyl)carbonyl]-O-(1-methylcyclopentyl)-L-threoninate (0.30 g, 0.78 mmol) in 10 mL of pyridine was treated with 2-isocyanato-1,3,5-trimethylbenzene (0.63 g, 3.90 mmol) for ca. 16 h at RT. The reaction was quenched with 1N HCl and extracted with ethyl acetate. The organic layer dried over magnesium sulfate, filtered, and the solvent evaporated to give 0.45 g of product as an amber semi-solid.

### Step 8

### O-(1-methylcyclopentyl)-N-{[3-({[(2,4,6-trimethylphenyl)amino]carbonyl}amino)-2-naphthalenyl]carbonyl}-L-threonine

Lithium hydroxide monohydrate (0.20 g, 8.35 mmol) was added to a solution of methyl O-(1-methylcyclopentyl)-N-{[3-({[(2,4,6-trimethylphenyl)amino]carbonyl}amino)-2-naphthalenyl]carbonyl}-L-threoninate (0.45 g, 0.824 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated in vacuo to give 0.084 g (19% yield) of product as a light orange solid. ES MS m/z 532 (M+H).

### Example 521

### N-{[4-fluoro-2-({[(2,4,6-trimethylphenyl)amino]carbonyl}amino)phenyl]carbonyl}-O-(phenylmethyl)-L-threonine

### Step 1

### Phenylmethyl N-[(2-amino-4-fluorophenyl)carbonyl]-O-(phenylmethyl)-L-threoninate

HATU (0.65 g, 1.71 mmol) was added to a solution of 2-amino-4-fluorobenzoic acid (0.22 g, 1.42 mmol), phenylmethyl O-(phenylmethyl)-L-threoninate (0.5 g, 1.67 mmol) and diisopropylethylamine (0.22 g, 1.72 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated to give 1.03 g of amber oil.

### Step 2

### Phenylmethyl N-{[4-fluoro-2-({[(2,4,6-trimethylphenyl)amino]carbonyl}amino)phenyl]carbonyl}-O-(phenylmethyl)-L-threoninate

Phenylmethyl N-[(2-amino-4-fluorophenyl)carbonyl]-O-(phenylmethyl)-L-threoninate (1.03 g, 2.36 mmol) in 10 mL of pyridine was treated with 2-isocyanato-1,3,5-trimethylbenzene (1.91 g, 11.82 mmol) for ca. 16 h at RT. The reaction was quenched with 1N HCl and extracted with ethyl acetate. The organic layer dried over magnesium sulfate, filtered, and the solvent evaporated to give 2.04 g of product as a light orange semi-solid.

### Step 3

### N-{[4-fluoro-2-({[(2,4,6-trimethylphenyl)amino]carbonyl}amino)phenyl]carbonyl}-O-(phenylmethyl)-L-threonine

Lithium hydroxide monohydrate (0.82 g, 34.24 mmol) was added to a solution of phenylmethyl N-{[4-fluoro-2-({[(2,4,6-trimethylphenyl)amino]carbonyl}amino)phenyl]carbonyl}-O-(phenylmethyl)-L-threoninate (2.04 g, 3.41 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated. Chromatography on silica gel with hexane/ethyl acetate gave 0.166 g of product. (10% yield) ES MS m/z 508 (M+H).

### Example 522

### N-{[3′,4′-difluoro-3-({[(2,4,6-trimethylphenyl)amino]carbonyl}amino)-4-biphenylyl]carbonyl}-O-(1,1-dimethylethyl)-D-threonine

### Step 1

### Methyl N-[(3′,4′-difluoro-3-nitro-4-biphenylyl)carbonyl]-O-(1,1-dimethylethyl)-D-threoninate

HATU (0.57 g, 1.50 mmol) was added to a solution of 3-amino-3′,4′-difluoro-4-biphenylcarboxylic acid (0.35 g, 1.25 mmol), methyl O-(1,1-dimethylethyl)-D-threoninate hydrochloride (0.34 g, 1.51 mmol) and diisopropylethylamine (0.19 g, 1.49 mmol) in 10 mL of DMF. The mixture was stirred at RT for ca. 15 h. The reaction was quenched with saturated sodium bicarbonate and diluted with ethyl acetate. The organic layer was dried over magnesium sulfate, filtered, and the solvent evaporated to give 0.88 g of yellow oil.

### Step 2

### Methyl N-[(3-amino-3′,4′-difluoro-4-biphenylyl)carbonyl]-O-(1,1-dimethylethyl)-D-threoninate

To a solution of methyl N-[(3′,4′-difluoro-3-nitro-4-biphenylyl)carbonyl]-O-(1,1-dimethylethyl)-D-threoninate (0.88 g, 1.95 mmol) in 20 ml of ethanol was added 8.8 ml of saturated ammonium chloride and indium (1.76 g, 15.33 mmol). The reaction was heated to reflux for 16 hours and then diluted with water and ethyl acetate. The organic layer was dried over magnesium sulfate filtered and concentrated. Chromatography on silica gel with hexane/ethyl acetate gave 0.33 g of yellow oil.

### Step 3

### Methyl N-{[3′,4′-difluoro-3-({[(2,4,6-trimethylphenyl)amino]carbonyl}amino)-4-biphenylyl]carbonyl}-O-(1,1-dimethylethyl)-D-threoninate

Methyl N-[(3-amino-3′,4′-difluoro-4-biphenylyl)carbonyl]-O-(1,1-dimethylethyl)-D-threoninate (0.33 g, 0.78 mmol) in 15 mL of pyridine was treated with 2-isocyanato-1,3,5-trimethylbenzene (0.63 g, 3.90 mmol) for ca. 16 h at RT. The reaction was quenched with 1N HCl and extracted with ethyl acetate. The organic layer dried over magnesium sulfate, filtered, and the solvent evaporated. Chromatography on silica gel with hexane/ethyl acetate gave 0.40 g of light yellow oil.

### Step 4

### N-{[3′,4′-difluoro-3-({[(2,4,6-trimethylphenyl)amino]carbonyl}amino)-4-biphenylyl]carbonyl}-O-(1,1-dimethylethyl)-D-threonine

Lithium hydroxide monohydrate (0.16 g, 6.68 mmol) was added to a solution of methyl N-{[3′,4′-difluoro-3-({[(2,4,6-trimethylphenyl)amino]carbonyl}amino)-4-biphenylyl]carbonyl}-O-(1,1-dimethylethyl)-D-threoninate (0.40 g, 0.69 mmol) in dioxane:water/10:1 (10 ml). The mixture was stirred at RT overnight. The reaction mixture was acidified with 1N aqueous HCl and extracted with ethyl acetate. The organic phase was dried over magnesium sulfate, filtered and concentrated to give 0.118 g of fluffy orange solid (30% yield) ES MS m/z 568 (M+H).

### Example 523

### (2S)-cyclohexyl({[2′-(methyloxy)-3-({[(2,4,6-trimethylphenyl)amino]carbonyl}amino)-4-biphenylyl]carbonyl}amino)ethanoic acid

### Step 1

### Methyl 2′-(methyloxy)-3-nitro-4-biphenylcarboxylate

A mixture of methyl 4-chloro-2-nitrobenzoate (0.500 g, 2.32 mmol), 2-methoxyphenylboronic acid (0.38 g, 2.55 mmol), trans-dichlorobis(tricyclohexylphosphine)palladium(II) (0.086 g, 0.12 mmol), cesium fluoride (1.05 g, 6.95 mmol), 1 mL of water and 6 mL of acetonitrile was heated in a microwave reactor at 150° C. for 5 minutes. The cooled reaction mixture was diluted with ethyl acetate, filtered through Celite, washed with water and dried over sodium sulfate. Chromatography on silica gel with hexane/ethyl acetate gave 0.571 g (86% yield) of desired product as a colorless oil

### Step 2

### 2′-(Methyloxy)-3-nitro-4-biphenylcarboxylic acid

Lithium hydroxide (0.457 g, 19.0 mmol) was added to a solution of methyl 2′-(methyloxy)-3-nitro-4-biphenylcarboxylate (0.547 g, 1.90 mmol) in 10 mL of THF: methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.517 g (99% yield) of desired product as a white solid.

### Step 3

### Methyl(2S)-cyclohexyl({[2′-(methyloxy)-3-nitro-4-biphenylyl]carbonyl}amino)ethanoate

HATU (0.473 g, 1.24 mmol) was added to a solution of 2′-(methyloxy)-3-nitro-4-biphenylcarboxylic acid (0.226 g, 0.83 mmol), methyl(2S)-amino(cyclohexyl)ethanoate hydrochloride (0.142 g, 0.83 mmol), and diisopropylethylamine (0.21 mL, 1.24 mmol) in 5 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.244 g (69% yield) g of desired product as a white solid.

### Step 4

### Methyl(2S)-({[3-amino-2′-(methyloxy)-4-biphenylyl]carbonyl}amino)(cyclohexyl)ethanoate

A mixture of methyl(2S)-cyclohexyl({[2′-(methyloxy)-3-nitro-4-biphenylyl]carbonyl}amino)ethanoate (0.242 g, 0.57 mmol) and 5% palladium on carbon (0.060 g, 0.028 mmol) in 20 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.219 g (97% yield) of desired product as an off-white solid.

### Step 5

### Methyl(2S)-cyclohexyl ({[2′-(methyl oxy)-3-({[(2,4,6-trimethylphenyl)amino]carbonyl}amino)-4-biphenylyl]carbonyl}amino)ethanoate

2,4,6-Trimethylphenylisocyanate (0.261 g, 0.54 mmol) was added to a solution of methyl(2S)-({[3-amino-2′-(methyloxy)-4-biphenylyl]carbonyl}amino)(cyclohexyl)ethanoate (0.214 g, 0.54 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.223 g (74% yield) of desired product as a white solid.

### Step 6

### (2S)-cyclohexyl({[2′-(methyloxy)-3-({[(2,4,6-trimethylphenyl)amino]carbonyl}amino)-4-biphenylyl]carbonyl}amino)ethanoic acid

Lithium hydroxide (0.091 g, 3.80 mmol) was added to a solution of methyl(2S)-cyclohexyl({[2′-(methyloxy)-3-({[(2,4,6-trimethylphenyl)amino]carbonyl}amino)-4-biphenylyl]carbonyl}amino)ethanoate (0.215 g, 0.38 mmol) in 5 mL of THF: methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.164 g (79% yield) of desired product as a white solid. ES MS m/z 544 (M+H)

### Example 524

### O-(1,1-Dimethylethyl)-N-{[2′-(methyloxy)-3-({[(2,4,6-trimethylphenyl)amino]carbonyl}amino)-4-biphenylyl]carbonyl}-L-threonine

### Step 1

### Methyl O-(1,1-dimethylethyl)-N-{[2′-(methyloxy)-3-nitro-4-biphenylyl]carbonyl}-L-threoninate

HATU (0.467 g, 1.23 mmol) was added to a solution of 2′-(methyloxy)-3-nitro-4-biphenylcarboxylic acid (0.224 g, 0.82 mmol), methyl O-(1,1-dimethylethyl)-L-threoninate hydrochloride (0.185 g, 0.82 mmol), and diisopropylethylamine (0.21 mL, 1.23 mmol) in 5 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.272 g (75% yield) g of desired product as a white solid.

### Step 2

### Methyl N-{[3-amino-2′-(methyloxy)-4-biphenylyl]carbonyl}-O-(1,1-dimethylethyl)-L-threoninate

A mixture of methyl O-(1,1-dimethylethyl)-N-{[2′-(methyloxy)-3-nitro-4-biphenylyl]carbonyl}-L-threoninate (0.268 g, 0.60 mmol) and 5% palladium on carbon (0.064 g, 0.030 mmol) in 20 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.178 g (72% yield) of desired product as a white solid.

### Step 3

### Methyl O-(1,1-dimethylethyl)-N-{[2′-(methyloxy)-3-({[(2,4,6-trimethylphenyl)amino]carbonyl}amino)-4-biphenylyl]carbonyl}-L-threoninate

2,4,6-Trimethylphenylisocyanate (0.204 g, 1.27 mmol) was added to a solution of methyl N-{[3-amino-2′-(methyloxy)-4-biphenylyl]carbonyl}-O-(1,1-dimethylethyl)-L-threoninate (0.175 g, 0.42 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.187 g (77% yield) of desired product as a white solid.

### Step 4

### O-(1,1-Dimethylethyl)-N-{[2′-(methyloxy)-3-({[(2,4,6-trimethylphenyl)amino]carbonyl}amino)-4-biphenylyl]carbonyl}-L-threonine

Lithium hydroxide (0.075 g, 3.13 mmol) was added to a solution of methyl O-(1,1-dimethylethyl)-N-{[2′-(methyloxy)-3-({[(2,4,6-trimethylphenyl)amino]carbonyl}amino)-4-biphenylyl]carbonyl}-L-threoninate (0.180 g, 0.31 mmol) in 5 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum. The residue was purified by chromatography on silica gel with dichloromethane:methanol to give 0.036 g (21% yield) of desired product. ES MS m/z 562 (M+H)

### Example 525

### N-{[3′,5′-Difluoro-3-({[(2,4,6-trimethylphenyl)amino]carbonyl}amino)-4-biphenylyl]carbonyl}-O-(1,1-dimethylethyl)-L-threonine

### Step 1

### Methyl 3′,5′-difluoro-3-nitro-4-biphenylcarboxylate

A mixture of methyl 4-chloro-2-nitrobenzoate (0.500 g, 2.32 mmol), 3,5-difluorophenylboronic acid (0.403 g, 2.55 mmol), trans-dichlorobis(tricyclohexylphosphine)palladium(II) (0.087 g, 0.12 mmol), cesium fluoride (1.06 g, 6.96 mmol), 1 mL of water and 6 mL of acetonitrile was heated in a microwave reactor at 150° C. for 5 minutes. The cooled reaction mixture was diluted with ethyl acetate, filtered through Celite, washed with water and dried over sodium sulfate. Chromatography on silica gel with hexane/ethyl acetate gave 0.562 g (83% yield) of desired product as a white solid.

### Step 2

### 3′,5′-Difluoro-3-nitro-4-biphenylcarboxylic acid

Lithium hydroxide (0.133 g, 5.53 mmol) was added to a solution of methyl 3′,5′-difluoro-3-nitro-4-biphenylcarboxylate (0.540 g, 1.84 mmol) in 10 mL of THF: methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.467 g (91% yield) of desired product as a white solid.

### Step 3

### Methyl N-[(3′,5′-difluoro-3-nitro-4-biphenylyl)carbonyl]-O-(1,1-dimethylethyl)-L-threoninate

HATU (0.471 g, 1.24 mmol) was added to a solution of 3′,5′-difluoro-3-nitro-4-biphenylcarboxylic acid (0.233 g, 0.83 mmol), methyl O-(1,1-dimethylethyl)-L-threoninate hydrochloride (0.188 g, 0.83 mmol), and diisopropylethylamine (0.22 mL, 1.24 mmol) in 5 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.273 g (73% yield) g of desired product as a white solid.

### Step 4

### Methyl N-[(3-amino-3′,5′-difluoro-4-biphenylyl)carbonyl]-O-(1,1-dimethylethyl)-L-threoninate

A mixture of methyl N-[(3′,5′-difluoro-3-nitro-4-biphenylyl)carbonyl]-O-(1,1-dimethylethyl)-L-threoninate (0.267 g, 0.59 mmol) and 5% palladium on carbon (0.063 g, 0.029 mmol) in 15 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.240 g (97% yield) of desired product as an off-white gum.

### Step 5

### Methyl N-{[3′,5′-difluoro-3-({[(2,4,6-trimethylphenyl)amino]carbonyl}amino)-4-biphenylyl]carbonyl}-O-(1,1-dimethylethyl)-L-threoninate

2,4,6-Trimethylphenylisocyanate (0.275 g, 1.71 mmol) was added to a solution of methyl N-[(3-amino-3′,5′-difluoro-4-biphenylyl)carbonyl]-O-(1,1-dimethylethyl)-L-threoninate (0.239 g, 0.57 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.259 g (78% yield) of desired product as a white solid.

### Step 6

### N-{[3′,5′-Difluoro-3-({[(2,4,6-trimethylphenyl)amino]carbonyl}amino)-4-biphenylyl]carbonyl}-O-(1,1-dimethylethyl)-L-threonine

Lithium hydroxide (0.105 g, 4.37 mmol) was added to a solution of methyl N-{[3′,5′-difluoro-3-({[(2,4,6-trimethylphenyl)amino]carbonyl}amino)-4-biphenylyl]carbonyl}-O-(1,1-dimethylethyl)-L-threoninate (0.254 g, 0.44 mmol) in 5 mL of THF: methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.252 g (100% yield) of desired product. ES MS m/z 566 (M−H)

### Example 526

### (2S)-Cyclohexyl({[3′,5′-difluoro-3-({[(2,4,6-trimethylphenyl)amino]carbonyl}amino)-4-biphenylyl]carbonyl}amino)ethanoic acid

### Step 1

### Methyl(2S)-cyclohexyl{[(3′,5′-difluoro-3-nitro-4-biphenylyl)carbonyl]amino}ethanoate

HATU (0.441 g, 1.16 mmol) was added to a solution of 3′,5′-difluoro-3-nitro-4-biphenylcarboxylic acid (0.215 g, 0.77 mmol), methyl(2S)-amino(cyclohexyl)ethanoate hydrochloride (0.160 g, 0.77 mmol), and diisopropylethylamine (0.20 mL, 1.16 mmol) in 5 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.287 g (86% yield) g of desired product as a white solid.

### Step 2

### Methyl(2S)-{[(3-amino-3′,5′-difluoro-4-biphenylyl)carbonyl]amino}(cyclohexyl)ethanoate

A mixture of methyl(2S)-cyclohexyl{[(3′,5′-difluoro-3-nitro-4-biphenylyl)carbonyl]amino}ethanoate (0.270 g, 0.62 mmol) and 5% palladium on carbon (0.067 g, 0.031 mmol) in 25 mL of ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.224 g (89% yield) of desired product as a beige gum.

### Step 3

### Methyl(2S)-cyclohexyl({[3′,5′-difluoro-3-({[(2,4,6-trimethylphenyl)amino]carbonyl}amino)-4-biphenylyl]carbonyl}amino)ethanoate

2,4,6-Trimethylphenylisocyanate (0.274 g, 1.64 mmol) was added to a solution of methyl(2S)-{[(3-amino-3′,5′-difluoro-4-biphenylyl)carbonyl]amino}(cyclohexyl)ethanoate (0.220 g, 0.55 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.278 g (90% yield) of desired product as a white solid.

### Step 4

### (2S)-Cyclohexyl({[3′,5′-difluoro-3-({[(2,4,6-trimethylphenyl)amino]carbonyl}amino)-4-biphenylyl]carbonyl}amino)ethanoic acid

Lithium hydroxide (0.115 g, 4.77 mmol) was added to a solution of methyl(2S)-cyclohexyl({[3′,5′-difluoro-3-({[(2,4,6-trimethylphenyl)amino]carbonyl}amino)-4-biphenylyl]carbonyl}amino)ethanoate (0.269 g, 0.48 mmol) in 5 mL of THF: methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.208 g (79% yield) of desired product as an off-white solid. APCI MS m/z 550 (M+H)

### Example 527

### O-(1,1-Dimethylethyl)-N-{[4′-fluoro-3-({[(2,4,6-trimethylphenyl)amino]carbonyl}amino)-4-biphenylyl]carbonyl}-L-threonine

### Step 1

### Methyl 4′-fluoro-3-nitro-4-biphenylcarboxylate

A mixture of methyl 4-chloro-2-nitrobenzoate (0.500 g, 2.32 mmol), 4-fluorophenylboronic acid (0.357 g, 2.55 mmol), trans-dichlorobis(tricyclohexylphosphine)palladium(II) (0.086 g, 0.12 mmol), cesium fluoride (1.06 g, 6.96 mmol), 1 mL of water and 6 mL of acetonitrile was heated in a microwave reactor at 150° C. for 5 minutes. The cooled reaction mixture was diluted with ethyl acetate, filtered through Celite, washed with water and dried over sodium sulfate. Chromatography on silica gel with hexane/ethyl acetate gave 0.537 g (84% yield) of desired product as a white solid.

### Step 2

### 4′-Fluoro-3-nitro-4-biphenylcarboxylic acid

Lithium hydroxide (0.137 g, 5.73 mmol) was added to a solution of methyl 4′-fluoro-3-nitro-4-biphenylcarboxylate (0.525 g, 1.91 mmol) in 10 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.465 g (93% yield) of desired product as a white solid.

### Step 3

### Methyl O-(1,1-dimethylethyl)-N-[(4′-fluoro-3-nitro-4-biphenylyl)carbonyl]-L-threoninate

HATU (0.502 g, 1.32 mmol) was added to a solution of 4′-Fluoro-3-nitro-4-biphenylcarboxylic acid (0.229 g, 0.88 mmol), methyl O-(1,1-dimethylethyl)-L-threoninate hydrochloride (0.198 g, 0.88 mmol), and diisopropylethylamine (0.23 mL, 1.32 mmol) in 5 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.300 g (79% yield) g of desired product as a white solid.

### Step 4

### Methyl N-[(3-amino-4-fluoro-4-biphenylyl)carbonyl]-O-(1,1-dimethylethyl)-L-threoninate

A mixture of methyl O-(1,1-dimethylethyl)-N-[(4′-fluoro-3-nitro-4-biphenylyl)carbonyl]-L-threoninate (0.294 g, 0.68 mmol) and 5% palladium on carbon (0.072 g, 0.034 mmol) in ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.264 g (96% yield) of desired product as a white solid.

### Step 5

### Methyl O-(1,1-dimethylethyl)-N-{[4′-fluoro-3-({[(2,4,6-trimethylphenyl)amino]carbonyl}amino)-4-biphenylyl]carbonyl}-L-threoninate

2,4,6-Trimethylphenylisocyanate (0.310 g, 1.92 mmol) was added to a solution of methyl N-[(3-amino-4′-fluoro-4-biphenylyl)carbonyl]-O-(1,1-dimethylethyl)-L-threoninate (0.258 g, 0.64 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.277 g (77% yield) of desired product as a white solid.

### Step 6

### O-(1,1-Dimethylethyl)-N-{[4′-fluoro-3-({[(2,4,6-trimethylphenyl)amino]carbonyl}amino)-4-biphenylyl]carbonyl}-L-threonine

Lithium hydroxide (0.115 g, 4.81 mmol) was added to a solution of methyl O-(1,1-dimethylethyl)-N-{[4′-fluoro-3-({[(2,4,6-trimethylphenyl)amino]carbonyl}amino)-4-biphenylyl]carbonyl}-L-threoninate (0.271 g, 0.48 mmol) in 5 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum. Chromatography on silica gel with hexane/ethyl acetate gave 0.131 g (50% yield) of desired product as a white solid. APCI MS m/z 550 (M+H).

### Example 528

### O-(1,1-Dimethylethyl)-N-{[3-({[(2,4,6-trimethylphenyl)amino]carbonyl}amino)-4-biphenylyl]carbonyl}-L-threonine

### Step 1

### Methyl 3-nitro-4-biphenylcarboxylate

A mixture of methyl 4-chloro-2-nitrobenzoate (1.00 g, 4.64 mmol), phenylboronic acid (0.623 g, 5.10 mmol), trans-dichlorobis(tricyclohexylphosphine)palladium(II) (0.171 g, 0.23 mmol), cesium fluoride (2.11 g, 13.9 mmol), 5 mL of water and 10 mL of acetonitrile was heated in a microwave reactor at 150° C. for 7 minutes. The cooled reaction mixture was diluted with ethyl acetate, filtered through Celite, washed with water and dried over sodium sulfate. Chromatography on silica gel with hexane/ethyl acetate gave 0.99 g (83% yield) of desired product as a white solid.

### Step 2

### 3-Nitro-4-biphenylcarboxylic acid

Lithium hydroxide (0.39 g, 16.4 mmol) was added to a solution of methyl 3-nitro-4-biphenylcarboxylate (0.423 g, 1.64 mmol) in 16 mL of THF:methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.383 g (96% yield) of desired product as a white solid.

### Step 3

### Methyl O-(1,1-dimethylethyl)-N-[(3-nitro-4-biphenylyl)carbonyl]-L-threoninate

HATU (0.448 g, 1.18 mmol) was added to a solution of 3-nitro-4-biphenylcarboxylic acid (0.192 g, 0.79 mmol), methyl O-(1,1-dimethylethyl)-L-threoninate hydrochloride (0.178 g, 0.79 mmol), and diisopropylethylamine (0.21 mL, 1.18 mmol) in 5 mL of DMF. The mixture was stirred at room temperature overnight, and then diluted with ethyl acetate, and washed with water and brine. The organic phase was dried over anhydrous sodium sulfate and the solvent was removed under vacuum to give 0.345 g of desired product as a white solid.

### Step 4

### Methyl N-[(3-amino-4-biphenylyl)carbonyl]-O-(1,1-dimethylethyl)-L-threoninate

A mixture of methyl O-(1,1-dimethylethyl)-N-[(3-nitro-4-biphenylyl)carbonyl]-L-threoninate (0.325 g, 0.78 mmol) and 5% palladium on carbon (0.083 g, 0.039 mmol) in ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.299 g (99% yield) of desired product as a white solid.

### Step 5

### Methyl O-(1,1-dimethylethyl)-N-{[3-({[(2,4,6-trimethylphenyl)amino]carbonyl}amino)-4-biphenylyl]carbonyl}-L-threoninate

2,4,6-Trimethylphenylisocyanate (0.368 g, 2.30 mmol) was added to a solution of methyl N-[(3-amino-4-biphenylyl)carbonyl]-O-(1,1-dimethylethyl)-L-threoninate (0.294 g, 0.76 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.292 g (70% yield) of desired product as a white solid.

### Step 6

### O-(1,1-Dimethylethyl)-N-{[3-({[(2,4,6-trimethylphenyl)amino]carbonyl}amino)-4-biphenylyl]carbonyl}-L-threonine

Lithium hydroxide (0.127 g, 0.53 mmol) was added to a solution of methyl O-(1,1-dimethylethyl)-N-{[3-({[(2,4,6-trimethylphenyl)amino]carbonyl}amino)-4-biphenylyl]carbonyl}-L-threoninate (0.288 g, 0.53 mmol) in 5 mL of THF: methanol:water/3:1:1. The mixture was stirred at room temperature overnight. The solvent was evaporated and 1N aqueous hydrochloric acid was added to the residue. The resulting suspension was extracted with ethyl acetate, dried over anhydrous sodium sulfate and the solvent removed under vacuum to give 0.227 g (81% yield) of desired product as a white solid. APCI MS m/z 532 (M+H).

### Example 529

### 1-({[3-({[(2,4,6-Trimethylphenyl)amino]carbonyl}amino)-4-biphenylyl]carbonyl}amino)cyclooctanecarboxylic acid

### Step 1

### Methyl 1-{[(3-nitro-4-biphenylyl)carbonyl]amino}cyclooctanecarboxylate

HATU (0.414 g, 1.09 mmol) was added to a solution of 3-nitro-4-biphenylcarboxylic acid (0.178 g, 0.73 mmol), methyl 1-aminocyclooctanecarboxylate hydrochloride (0.162 g, 0.73 mmol), and diisopropylethylamine (0.19 mL, 1.09 mmol) in 5 mL of DMF. The mixture was stirred at room temperature overnight, and then diluted with ethyl acetate, and washed with water and brine. The organic phase was dried over anhydrous sodium sulfate and the solvent was removed under vacuum. Chromatography on silica gel with hexane/ethyl acetate gave 0.163 g (54% yield) of desired product as a white solid.

### Step 2

### Methyl 1-{[(3-amino-4-biphenylyl)carbonyl]amino}cyclooctanecarboxylate

A mixture of methyl 1-{[(3-nitro-4-biphenylyl)carbonyl]amino}cyclooctanecarboxylate (0.159 g, 0.39 mmol) and 5% palladium on carbon (0.041 g, 0.019 mmol) in ethanol in a pressure reaction vessel was evacuated and flushed with nitrogen three times, then evacuated and filled with 50 psi of hydrogen and stirred for one hour. The reaction vessel was then evacuated and flushed with nitrogen. The mixture was filtered through Celite and the filtrate was evaporated to give 0.116 g (78% yield) of desired product as a colorless resin.

### Step 3

### Methyl 1-({[3-({[(2,4,6-trimethylphenyl)amino]carbonyl}amino)-4-biphenylyl]carbonyl}amino)cyclooctanecarboxylate

2,4,6-Trimethylphenylisocyanate (0.147 g, 0.91 mmol) was added to a solution of methyl 1-{[(3-amino-4-biphenylyl)carbonyl]amino}cyclooctanecarboxylate (0.116 g, 0.305 mmol) in 5 mL of anhydrous pyridine. The mixture was stirred at room temperature overnight. Pyridine was removed under vacuum and ethyl acetate was added to the residue. The insoluble material was filtered off, the filtrate was washed with 1N aqueous HCl, dried over anhydrous sodium sulfate and the solvent evaporated under reduced pressure. Chromatography on silica gel with hexane/ethyl acetate gave 0.129 g (78% yield) of desired product as a white solid.

### Step 4

### 1-({[3-({[(2,4,6-Trimethylphenyl)amino]carbonyl}amino)-4-biphenylyl]carbonyl}amino)cyclooctanecarboxylic acid

Lithium hydroxide (0.054 g, 2.24 mmol) was added to a solution of methyl 1-({[3-({[(2,4,6-trimethylphenyl)amino]carbonyl}amino)-4-biphenylyl]carbonyl}amino)cyclooctanecarboxylate (0.121 g, 0.22 mmol) in 5 mL of THF:methanol:water/3:1:1. The mixture was heated at 60° C. for 6 hours. The solvent was evaporated and 1N aqueous hydrochloric acid was added to the residue. The resulting suspension was extracted with ethyl acetate, dried over anhydrous sodium sulfate and the solvent removed under vacuum to give 0.085 g (73% yield) of desired product as a white solid. APCI MS m/z 528 (M+H).

### Example 530

### N-{[3-({[(4-Cyclopropyl-2,6-dimethylphenyl)amino]carbonyl}amino)-3′-fluoro-4-biphenylyl]carbonyl}-O-(1,1-dimethylethyl)-L-threonine

### Step 1

### Methyl N-{[3-({[(4-cyclopropyl-2,6-dimethylphenyl)amino]carbonyl}amino)-3′-fluoro-4-biphenylyl]carbonyl}-O-(1,1-dimethylethyl)-L-threoninate

A mixture of methyl N-[(3-amino-3′-fluoro-4-biphenylyl)carbonyl]-O-(1,1-dimethylethyl)-L-threoninate (0.216 g, 0.54 mmol), 5-cyclopropyl-2-isocyanato-1,3-dimethylbenzene (0.120 g, 0.64 mmol), and triethylamine (0.15 mL, 1.08 mmol) in 3 mL of DMF was heated at 70° C. for 3 hours. An additional 0.100 g of isocyanate was added and the mixture was heated for another hour. The reaction mixture was cooled to room temperature and the DMF was removed under vacuum. Chromatography on silica gel with hexane/ethyl acetate gave a mixture containing 65% of the desired product. This mixture was carried on to the next step without further purification.

### Step 2

### N-{[3-({[(4-Cyclopropyl-2,6-dimethylphenyl)amino]carbonyl}amino)-3′-fluoro-4-biphenylyl]carbonyl}-O-(1,1-dimethylethyl)-L-threonine

The product from Step 1 containing ca 65% of methyl N-{[3-({[(4-cyclopropyl-2,6-dimethylphenyl)amino]carbonyl}amino)-3′-fluoro-4-biphenylyl]carbonyl}-O-(1,1-dimethylethyl)-L-threoninate (0.165 g, 0.28 mmol) was dissolved in 5 mL of THF:methanol:water/3:1:1 and 0.067 g (2.80 mmol) of lithium hydroxide was added. The mixture was stirred at room temperature overnight. The solvent was evaporated and the residue was treated with aqueous 1N hydrochloric acid. The resulting suspension was extracted with ethyl acetate. The organic phase was dried over sodium sulfate and the solvent was evaporated. The residue was subjected to chromatography with dichloromethane/methanol, and hexane/ethyl acetate to give 0.052 g of a mixture containing ca 84% of desired product and an amine byproduct. To a solution of this material in 2 mL of tetrahydrofuran was added 0.090 g (0.12 mmol) of MP-isocyanate resin. The mixture was heated at 60° C. for 18 hours, cooled to room temperature and filtered. The filtrate was evaporated to dryness to give 0.032 g of desired product as a yellow solid. APCI MS m/z 576 (M+H).

### Example 531

### (2S)-cyclohexyl({[3-({[(4-cyclopropylphenyl)amino]carbonyl}amino)-2-naphthalenyl]carbonyl}amino)ethanoic acid

### Step 1

### N-(4-bromo-2,6-dimethylphenyl)-2,2,2-trifluoroacetamide

To a solution of 4-bromo-2,6-dimethylaniline (4.0 g, 20.0 mmol) in 50 mL CH₂Cl₂ cooled to 0° C. was added Hunig's base (6.97 mL, 40 mmol) followed by the addition of trifluoroacetic anhydride (0.294 mL, 2.1 mmol). After stirring for an hour, the contents were washed with water, dried (K₂CO₃) and then concentrated under vacuum to afford the crude product (4.5 g, 76% yield).

### Step 2

### N-(4-cyclopropyl-2,6-dimethylphenyl)-2,2,2-trifluoroacetamide

To a solution of N-(4-bromo-2,6-dimethylphenyl)-2,2,2-trifluoroacetamide (4.5 g, 15.2 mmol) in DME (90 mL) was added 2-cyclopropyl-4,4,5,5-tetramethyl-1,3,2-dioxaborolane (3.06 g, 18.24 mmol) followed by the addition of bistriphenylphosphine Pd (II) dichloride (1.1 g, 10 mol %) and 2M Na₂CO₃ (30 mL). The contents were refluxed for 48 h. Concentration followed by loading of the crude reaction onto an isco column eluting with EtOAc/Hexane (0-30%) gave a white solid (3.1 g, 80% yield).

### Step 3

### (4-cyclopropyl-2,6-dimethylphenyl)amine 4-cyclopropyl-2,6-dimethylaniline

To a solution of N-(4-cyclopropyl-2,6-dimethylphenyl)-2,2,2-trifluoroacetamide (1.0 g, 3.89 mmol) in dioxane (20 mL) was added 4N NaOH (5 mL) and then contents refluxed for 6 h. The reaction was cooled and then followed by the addition of EtOAc. Separation of the organic layer followed by drying (MgSO₄) and concentration under vacuum gave the amine which was taken crude to the next step.

### Step 4

### 5-cyclopropyl-2-isocyanato-1,3-dimethylbenzene

To a solution of (4-cyclopropyl-2,6-dimethylphenyl)amine 4-cyclopropyl-2,6-dimethylaniline (0.551 g, 3.42 mmol) in CH₂Cl₂ (10 mL) cooled to 0° C. was added pyridine (0.828 mL, 10.26 mmol) followed by 2.0M solution of phosgene (2.56 mL, 4.78 mmol) in toluene. After warming to rt. The contents were stirred for 16 h. Addition of 1N HCl (30 mL) followed by separation of the organic layer, drying (MgSO₄) and concentration under vacuum gave the desired product.

### Step 5

### methyl(2S)-cyclohexyl({[3-({[(4-cyclopropylphenyl)amino]carbonyl}amino)-2-naphthalenyl]carbonyl}amino)ethanoate

To a solution of methyl(2S)-{[(3-amino-2-naphthalenyl)carbonyl]amino}(cyclohexyl)ethanoate hydrochloride salt (0.187 g, 0.5 mmol) in DMF (3.0 mL) was added 5-cyclopropyl-2-isocyanato-1,3-dimethylbenzene (0.112 g, 0.6 mmol) followed by triethyl amine (0.210 mL, 1.5 mmol) and the contents heated at 70 C for 2 h. The reaction was loaded onto an isco column and leuted with EtOAc/Hexane (0-60%) to afford 0.250 g (93%) of the product as a yellow solid.

### Step 6

### (2S)-cyclohexyl({[3-({[(4-cyclopropylphenyl)amino]carbonyl}amino)-2-naphthalenyl]carbonyl}amino)ethanoic acid

To a solution of methyl(2S)-cyclohexyl({[3-({[(4-cyclopropylphenyl)amino]carbonyl}amino)-2-naphthalenyl]carbonyl}amino)ethanoate (0.30 g, 0.569 mmol) in THF (2.0 mL) was added 1.0 M LiOH (1.99 mL, 1.99 mmol) and the contents stirred at rt. For 16 h. The reaction mixture was acidified to pH=4.0 followed by extraction with EtOAc. Drying with MgSO₄ followed by concentration under vacuum gave the product as a yellow solid (0.250 g, 98%). ES m/z 514 (M+H).

### Example 532

### N-{[3-({[(4-cyclopropyl-2,6-dimethylphenyl)amino]carbonyl}amino)-4′-(methyloxy)-4-biphenylyl]carbonyl}-O-(1,1-dimethylethyl)-L-threonine

### Step 1

### methyl N-{[3-({[(4-cyclopropyl-2,6-dimethylphenyl)amino]carbonyl}amino)-4′-(methyloxy)-4-biphenylyl]carbonyl}-O-(1,1-dimethylethyl)-L-threoninate

To a solution of methyl N-{[3-amino-4′-(methyloxy)-4-biphenylyl]carbonyl}-O-(1,1-dimethylethyl)-L-threoninate (0.207 g, 0.5 mmol) in DMF (3.0 mL) was added 5-cyclopropyl-2-isocyanato-1,3-dimethylbenzene (0.112 g, 0.6 mmol) followed by triethyl amine (0.210 mL, 1.5 mmol) and the contents heated at 700° C. for 2 h. The reaction was loaded onto an isco column and leuted with EtOAc/Hexane (0-60%) to afford 0.160 g (53%) of the product as a yellow solid.

### Step 2

### (2S)-cyclohexyl({[3-({[(4-cyclopropyl-2,6-dimethylphenyl)amino]carbonyl}amino)-4′-(methyloxy)-4-biphenylyl]carbonyl}amino)ethanoic acid

To a solution of methyl N-{[3-({[(4-cyclopropyl-2,6-dimethylphenyl)amino]carbonyl}amino)-4′-(methyloxy)-4-biphenylyl]carbonyl}-O-(1,1-dimethylethyl)-L-threoninate (0.110 g, 0.183 mmol) in THF (2.0 mL) was added 1.0 M LiOH (0.640 mL, 0.640 mmol) and the contents stirred at rt. For 16 h. The reaction mixture was acidified to pH=4.0 followed by extraction with EtOAc. Drying with MgSO₄ followed by concentration under vacuum gave the product as a yellow solid (0.096 g, 90%). ES m/z 588 (M+H).

### Example 533

### 1-({[5-(4-Chlorophenyl)-3-({[(2,4,6-trimethylphenyl)amino]carbonyl}amino)-2-thienyl]carbonyl}amino)cyclohexanecarboxylic acid

### Step 1

### 3-Amino-5-(4-chlorophenyl)-2-thiophenecarboxylic acid

Methyl 3-amino-5-(4-chlorophenyl)-2-thiophenecarboxylate (3.28 g, 12.24 mmol) was dissolved in dioxane (50 mL) and 1 M lithium hydroxide was added. The reaction was heated to 100° C. and stirred overnight. Cooled to rt and acidified with 1N HCl. Precipitate was collected and triturated with ethyl acetate to afford 2.89 g (11.42 mmol, 93%) of product as a yellow solid.

### Step 2

### Methyl 1-({[3-amino-5-(4-chlorophenyl)-2-thienyl]carbonyl}amino)cyclohexanecarboxylate

3-Amino-5-(4-chlorophenyl)-2-thiophenecarboxylic acid (2.039 g, 8.06 mmol), methyl 1-aminocyclohexanecarboxylate (1.555 g, 8.06 mmol) and triethyl amine (4.2 mL, 24.18 mmol) were dissolved in DMF (50 mL). HATU (4.59 g, 12.09 mmol) was added and the reaction stirred overnight. Diluted with ethyl acetate (100 mL) and washed with water (2×100 mL), brine (1×100 mL), dried over MgSO₄, filtered and concentrated. Purified on an ISCO (0-25% EtOAc in Hexane over 20 min) to afford 0.300 g (0.765 mmol, 9%) of the product as a yellow foam.

### Step 3

### 1-({[5-(4-Chlorophenyl)-3-({[(2,4,6-trimethylphenyl)amino]carbonyl}amino)-2-thienyl]carbonyl}amino)cyclohexanecarboxylic acid

Methyl 1-({[3-amino-5-(4-chlorophenyl)-2-thienyl]carbonyl}amino)cyclohexanecarboxylate (0.30 g, 0.76 mmol) was suspended in pyridine. 2-Isocyanato-1,3,5-trimethylbenzene (0.369 g, 2.29 mmol) was added. The reaction was stirred overnight at rt. Methanol (10 mL) was added and the reaction stirred for 30 min. Reaction was filtered and organics diluted with EtOAc (50 mL) and 1N HCl (25 mL). A precipitate was formed and collected. Purified on a chromatatron (5% MeOH in CH₂Cl₂) to give an impure material. The material was taken up in dioxane (2 mL) and 1 M lithium hydroxide (2 mL) was added. Heated to 100° C. and stirred for 1 h. Cooled to rt and acidified with 1N HCl and diluted with EtOAc (40 mL). Organics were washed with water (2×50 mL), dried over MgSO₄, filtered and concentrated. Attempted to dissolve in methylene chloride, but and insoluble white solid remained. Collected solid to afford 0.123 g (0.228 mmol, 30%) of the titled product. ES MS m/z 540 (M+H), 538 (M−H).

### Example 534

### 1-({[5-(3,4-difluorophenyl)-3-({[(2,4,6-trimethylphenyl)amino]carbonyl}amino)-2-thienyl]carbonyl}amino)cyclohexanecarboxylic acid

### Step 1

### (2Z)-3-chloro-3-(3,4-difluorophenyl)-2-propenenitrile

DMF (30 mL) was cooled to 0° C. and phosphorousoxychloride (6.72 mL, 72 mmol) was added. The reaction was stirred at 0° C. for 10 min and then 1-(3,4-difluorophenyl)ethanone (6.61 g, 42.9 mmol) was added. The reaction was warmed to rt and then heated to 50° C. for 10 mins. The reaction was cooled to 0° C. and hydroxylamine hydrochloride (11.78 g, 170 mmol) was added slowly. After stirring at rt for 5 mins, the reaction was heated to 120° C. for 15 mins. The reaction was cooled to rt and diluted with EtOAc and neutralized with satd. NaHCO₃. The organics were removed and the aqueous layer extracted with EtOAc. The combined organics were dried and concentrated. Carried forward without further purification.

### Step 2

### Methyl 3-amino-5-(3,4-difluorophenyl)-2-thiophenecarboxylate

To methanol (80 mL) was added sodium methoxide (11.71 mL of a 25% sol in MeOH) and methyl mercaptoacetate (3.76 mL, 30 mmol). (2Z)-3-Chloro-3-(3,4-difluorophenyl)-2-propenenitrile (8.3 g, 41.7 mmol) in DMF (30 mL) was added. The reaction was stirred at rt for 30 min. Water was added and the precipitate was collected. Solids were dried under vacuum to give 2.747 g (10.21 mmol, 24%) of the product as a light brown solid.

### Step 3

### 3-Amino-5-(3,4-difluorophenyl)-2-thiophenecarboxylic acid

Methyl 3-amino-5-(3,4-difluorophenyl)-2-thiophenecarboxylate (1.126 g, 4.19 mmol) was dissolved in dioxane (25 mL) and 1 M lithium hydroxide was added. The reaction was heated to 100° C. and stirred overnight. Cooled to rt, diluted with EtOAc (100 mL) and acidified with 1N HCl. Organics were washed with water (2×100 mL), brine (1×100 mL), dried over MgSO₄, filtered and concentrated to give 0.9847 g (3.86 mmol, 92%) of product as a yellow solid.

### Step 4

### Methyl 1-({[3-amino-5-(3,4-difluorophenyl)-2-thienyl]carbonyl}amino)cyclohexanecarboxylate

3-Amino-5-(3,4-difluorophenyl)-2-thiophenecarboxylic acid (0.985 g, 3.86 mmol), methyl 1-aminocyclohexanecarboxylate (0.745 g, 3.86 mmol) and triethyl amine (2 mL, 11.58 mmol) were dissolved in DMF (10 mL). HATU (2.201 g, 5.79 mmol) was added and the reaction stirred overnight. Diluted with ethyl acetate (100 mL) and washed with water (2×100 mL), brine (1×100 mL), dried over MgSO₄, filtered and concentrated. Purified on an ISCO (0-25% EtOAc in Hexane over 20 min) to afford 0.383 g (0.97 mmol, 25%) of the product as a white solid.

### Step 5

### 1-({[5-(3,4-difluorophenyl)-3-({[(2,4,6-trimethylphenyl)amino]carbonyl}amino)-2-thienyl]carbonyl}amino)cyclohexanecarboxylic acid

Methyl 1-({[3-amino-5-(3,4-difluorophenyl)-2-thienyl]carbonyl}amino)cyclohexanecarboxylate (0.383 g, 0.97 mmol) was suspended in pyridine. 2-Isocyanato-1,3,5-trimethylbenzene (0.270 g, 2.92 mmol) was added. The reaction was stirred overnight at rt. Methanol (10 mL) was added and the reaction stirred for 30 min. Reaction was filtered and organics diluted with EtOAc (50 mL) and 1N HCl (25 mL). A precipitate was formed and collected. The crude material was taken up in dioxane (2 mL) and 1 M lithium hydroxide (2 mL) was added. Heated to 100° C. and stirred for 1 h. Cooled to rt and acidified with 1N HCl and diluted with EtOAc (40 mL). Organics were washed with water (2×50 mL), dried over MgSO₄, filtered and concentrated. Purified on a chromatatron (5% MeOH in CH₂Cl₂) to afford 0.163 g (0.301 mmol, 31%) of the titled product. ES MS m/z 542 (M+H), 540 (M−H).

### Example 535

### 1-({[5-(3,4,5-trifluorophenyl)-3-({[(2,4,6-trimethylphenyl)amino]carbonyl}amino)-2-thienyl]carbonyl}amino)cyclohexanecarboxylic acid

### Step 1

### (2Z)-3-chloro-3-(3,4,5-trifluorophenyl)-2-propenenitrile

DMF (30 mL) was cooled to 0° C. and phosphorous oxychloride (9.89 mL, 106 mmol) was added. The reaction was stirred at 0° C. for 10 min and then 1-(3,4,5-trifluorophenyl)ethanone (7.37 g, 62.69 mmol) was added. The reaction was warmed to rt and then heated to 50° C. for 10 mins. The reaction was cooled to 0° C. and hydroxylamine hydrochloride (11.78 g, 170 mmol) was added slowly. After stirring at rt for 5 mins, the reaction was heated to 120° C. for 15 mins. The reaction was cooled to rt and diluted with EtOAc and neutralized with satd. NaHCO₃. The organics were removed and the aqueous layer extracted with EtOAc. The combined organics were dried and concentrated. Carried forward without further purification.

### Step 2

### Methyl 3-amino-5-(3,4,5-trifluorophenyl)-2-thiophenecarboxylate

To methanol (80 mL) was added sodium methoxide (2.33 g, 43.32 mmol) and methyl mercaptoacetate (3.06 mL, 30 mmol). (2Z)-3-Chloro-3-(3,4-difluorophenyl)-2-propenenitrile (7.45 g, 36.1 mmol) in DMF (30 mL) was added. The reaction was stirred at rt for 30 min. Water was added and the precipitate was collected. Solids were dried under vacuum to give 2.46 g (8.571 mmol, 14%) of the product as a light brown solid.

### Step 3

### 3-Amino-5-(3,4,5-trifluorophenyl)-2-thiophenecarboxylic acid

Methyl 3-amino-5-(3,4,5-trifluorophenyl)-2-thiophenecarboxylate (0.45 g, 1.57 mmol) was dissolved in dioxane (25 mL) and 1 M lithium hydroxide was added. The reaction was heated to 100° C. and stirred overnight. Cooled to rt, diluted with EtOAc (100 mL) and acidified with 1N HCl. Organics were washed with water (2×100 mL), brine (1×100 mL), dried over MgSO4, filtered and concentrated to give 0.358 g (1.31 mmol, 83%) of product as a yellow solid.

### Step 4

### Methyl 1-({[3-amino-5-(3,4,5-trifluorophenyl)-2-thienyl]carbonyl}amino)cyclohexanecarboxylate

3-Amino-5-(3,4,5-trifluorophenyl)-2-thiophenecarboxylic acid (0.358 g, 1.31 mmol), methyl 1-aminocyclohexanecarboxylate (0.253 g, 1.31 mmol) and triethyl amine (0.68 mL, 3.93 mmol) were dissolved in DMF (10 mL). HATU (0.746 g, 1.96 mmol) was added and the reaction stirred overnight. Diluted with ethyl acetate (100 mL) and washed with water (2×100 mL), brine (1×100 mL), dried over MgSO₄, filtered and concentrated. Purified on an ISCO (0-25% EtOAc in Hexane over 20 min) to afford 0.245 g (0.59 mmol, 45%) of the product as a white solid.

### Step 5

### 1-({[5-(3,4,5-trifluorophenyl)-3-({[(2,4,6-trimethylphenyl)amino]carbonyl}amino)-2-thienyl]carbonyl}amino)cyclohexanecarboxylic acid

Methyl 1-({[3-amino-5-(3,4,5-trifluorophenyl)-2-thienyl]carbonyl}amino)cyclohexanecarboxylate (0.245 g, 0.59 mmol) was suspended in pyridine. 2-Isocyanato-1,3,5-trimethylbenzene (0.287 g, 1.78 mmol) was added. The reaction was stirred overnight at rt. Methanol (10 mL) was added and the reaction stirred for 30 min. Reaction was filtered and organics diluted with EtOAc (50 mL) and 1N HCl (25 mL). A precipitate was formed and collected. The crude material was taken up in dioxane (2 mL) and 1 M lithium hydroxide (2 mL) was added. Heated to 100° C. and stirred for 1 h. Cooled to rt and acidified with 1N HCl and diluted with EtOAc (40 mL). Organics were washed with water (2×50 mL), dried over MgSO₄, filtered and concentrated to afford 0.276 g (0.49 mmol, 84%) of the product as a yellow foam. ES MS m/z 560 (M+H), 582 (M+Na) 558 (M−H).

### Biological Protocols

The utility of the compounds of Formula 1, a salt, solvate, or physiologically functional derivative thereof, in the treatment or prevention of diseases (such as detailed herein) in animals, particularly mammals (e.g., humans) may be demonstrated by the activity in conventional assays known to one of ordinary skill in the relevant art, including the in vitro assays described below.

The purified glycogen phosphorylase (GP) enzyme, wherein glycogen phosphorylase is in the activated “a” state, referred to as human liver glycogen phosphoarylase a (HLGPa), can be obtained according to the following procedures.

Appropriate Cloning and Expression of Human Liver Glycogen Phosphorylase

Human liver glycogen phosphorylase cDNA was amplified by polymerase chain reaction (PCR) from a commercially available human liver cDNA library (BD Biosciences). The cDNA was amplified as 2 overlapping fragments using the primers 5′GGCGAAGCCCCTGACAGACCAGGAGAAG3′ with 5′CGATGTCTGAGTGGATTTTAGCCACGCC3′ and 5′GGATATAGAAGAGTTAGAAGAAATTG3′ with 5′GGAAGCTTATCAATTTCCATTGACTTTGTTAGATTCATTGG3′. PCR conditions were 94° C. 1 min., 55° C. 1 min., 72° C. 2 min. for 40 cycles using the enzyme Pfu Turbo (Stratagene), 0.5% DMSO, 250 uM each nucleotide triphosphate, and 0.4 uM each primer plus the buffer recommended by the polymerase manufacturer. Each PCR fragment was molecularly cloned and the DNA sequence of each insert was determined. The 2 DNA fragments of the glycogen phosphorylase cDNA were then joined together in a bacterial expression plasmid, pTXK1007LTev (GlaxoSmithKline), creating a full-length cDNA fused at the 5′ end to codons for methionine-glycine-alanine-histidine-histidine-histidine-histidine-histidine-histidine-glycine-glycine-glutamate-asparagine-leucine-tyrosine-phenylalanine-glutamine-glycine-glycine-. The protein product would have a 6Xhistidine tag followed by a Tev protease cleavage site. The DNA sequence of both strands of the cDNA in pTXK1007LTev was determined.

Purification of Human Liver Glycogen Phosphorylase

The frozen cell paste (100 g) was thawed and suspended in 1200 ml of 50 mM Tris, 100 mM NaCl, 15 mM imidazole, pH 8.0. The cells were disrupted gently with a Polytron (Brinkmann, PT10-35), and passed twice through an AVP homogenizer. The E. coli cell lysates were clarified by centrifugation at 27,500×g for 45 minutes and filtered through a 0.8 micron filter. The solution was applied to a 21 ml Ni-NTA Superflow (Qiagen) column (ID 26 mm×H 4.0 cm) pre-equilibrated with 50 mM Tris, 100 mM NaCl, and 15 mM imidazole, pH 8.0. The column was washed with equilibration buffer until the A280 returned to baseline. The weakly bound proteins were eluted from the column with 10 bed column volumes of 50 mM imidazole in the same buffer. The glycogen phosphorylase was eluted with steps of 100 mM and 250 mM imidizole. Both the 100 mM and 250 mM fractions were pooled and then diluted 5 fold with 50 mM Tris, pH 8.0 buffer. This solution was loaded on a 21 ml Q fast flow column (Amersham Pharmacia Biotech AB, ID 2.6 cm×H 4.0 cm) pre-equilibrated with 50 mM Tris, pH 8.0. Glycogen phosphorylase was eluted with a continuous gradient from 0-30% of 1 M NaCl in 50 mM Tris, pH 8.0 (buffer B). Fractions of purified glycogen phosphorylase between 15% and 20% buffer B were pooled, aliquoted into microfuge tubes, and stored at −80° C. The purified fraction formed a single ˜100 kd band on a SDS-PAGE gel.

Activation of Human Liver Glycogen Phosphorylase

The activation of human liver glycogen phosphorylase (i.e., conversion of the inactive HLGPb form to the activated HLGPa form) was achieved by phosphorylating HLGPb with immobilized phosphorylase kinase.

10 mg of phosphorylase kinase (Sigma, P-2014) was dissolved in 2.5 ml of 100 mM HEPES, 80 mM CaCl2 (pH 7.4) and gently mixed with 1 ml of Affi-Gel (Active Ester Agarose, BioRad # 153-6099) beads previously equilibrated in the same buffer. The mixture was rocked 4 hours at 4° C. The beads were washed once with the same buffer and blocked for 1 hour at room temperature with a solution of 50 mM HEPES, 1 M glycine methyl ester, pH 8.0. The beads were then washed with 50 mM HEPES, 1 mM β-mercaptoethanol, pH 7.4 and stored at 4° C.

Frozen purified glycogen phosphorylase (HLGPb) was thawed in at 4° C. then dialyzed overnight into 50 mM HEPES, 100 mM NaCl, pH 7.4.15 mg of the dialyzed HLGPb, 3 mM ATP and 5 mM MgCl2 was incubated with 500 ul of the prepared Affi-Gel immobilized phosphorylase kinase beads equilibrated with 50 mM HEPES, 100 mM NaCl, pH 7.4. The degree of phosphorylation was monitored by following the increase in activity at 10 minute intervals using a modification of the assay system outlined below. Briefly, the assay contained 0.1 uM human liver glycogen phosphorylase, 50 mM HEPES, 100 mM KCl, 2.5 mM EGTA, MgCl₂, 3.5 mM KH₂PO₄, 0.5 mM DTT, 0.4 mg/mL glycogen, 7.5 mM Glucose, 0.50 mM β-nicotinamide adenine dinucleotide (β-NAD), 3 U/mL phosphoglucomutase, and 5 U/mL glucose-6-phosphate dehydrogenase, Activity was monitored by following the reduction of NAD⁺ at 340 nm. The reaction was stopped by removal of the beads from the mixture when no further increase in activity was observed (30-60 minutes). Phosphorylation was further confirmed by analysis of the sample by mass spectroscopy. The supernatant containing the activated sample was dialyzed in 50 mM HEPES, 100 mM NaCl, pH 7.4 overnight. The final sample was mixed with an equal volume of glycerol, aliquoted into microfuge tubes and stored at −20° C.

Human Liver Glycogen Phosphorylase a Enzymatic Activity Assay

An enzymatic assay was developed to measure the response of the activated form of glycogen phosphorylase (HLGPa) to small molecule (<1000 Da.) compounds. The assay was configured to monitor the pharmacologically relevant glycogenolytic reaction by coupling the production of glucose-1-phosphate from glycogen and inorganic phosphate to phosphoglucomutase, glucose-6-phosphate dehydrogenase, NADH oxidase and horseradish peroxidase to produce the fluorescent product resorufin. The concentrations of the reagent components were as follows: 5-25 nM human liver glycogen phosphorylase a, 1 mg/mL glycogen, 5 mM K₂HPO₄, 20 U/mL phosphoglucomutase (Sigma), 20 U/mL glucose-6-phosphate dehydrogenase (Sigma), 200 nM Thermus thermophilus NADH oxidase (prepared as described in Park, H. J.; Kreutzer, R.; Reiser, C. O. A.; Sprinzl, M. Eur. J. Biochem. 1992, 205, 875-879.), 2 U/mL horseradish peroxidase (Sigma), 30 uM FAD, 250 uM NAD⁺, +/−0.05% casein and 0.05% CHAPS as indicated, 100 mM NaCl, 50 uM amplex red, 10 mM glucose. The base assay buffer used was 50 mM HEPES, pH 7.6. To aid in the identification of glucose-sensitive inhibitors of glycogen phosphorylase, the assay was performed with and without 10 mM glucose. In order to scrub the assay of contaminating components that may contribute to non-HLGPa specific resorufin production, the reagents were prepared as two 2× concentrated cocktails. A solution of catalase-coated agarose beads is prepared in the base assay buffer. The first cocktail (cocktail #1) consisted of Thermus thermophilus NADH oxidase, NAD⁺, glycogen, phosphoglucomutase, glucose-6-phosphate dehydrogenase, K₂HPO₄, FAD, and 50 U/mL catalase-coated agarose beads. Amplex red was added to this solution after incubation at 25 C for 30 minutes and the catalase-coated agarose beads were removed by centrifugation and retention of supernatant. The second cocktail (cocktail #2) contained human liver glycogen phosphorylase-a and horseradish peroxidase (with and without glucose). The assays were performed with preincubation of compounds of this invention with cocktail #2 for 15 minutes, followed by the addition of cocktail #1 to initiate the reaction. The assays were performed in duplicate in 96 (black ½ volume Costar) or 384-well microtiter plates (small volume black Greiner) and the change in fluorescence due to product formation was measured on a fluorescence plate reader (Viewlux, Perkin Elmer, Molecular Devices) using a 525 nm excitation filter and 595 emission filter (ex560/em590 nm for Molecular devices).

TABLE 1

| | | | ESMS | IC₅₀ |
|---|---|---|---|---|
| Ex # | Structure | Chemical Name | +m/z | (uM) |
| 1 | 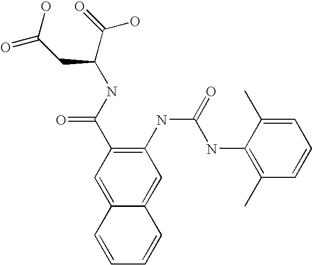 | N-[3-({[(2,6- dimethylphenyl)amino]carbonyl}amino)-2-naphthoyl]- L-aspartic acid | 450.4 | 0.46 |
| 2 | 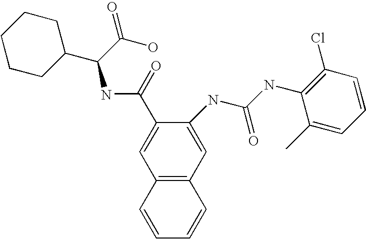 | (2S)-{[3-({[(2-chloro-6- methylphenyl) amino]carbonyl}amino)-2- naphthoyl]amino}(cyclohexyl) acetic acid | 493.2 | 0.12 |
| 3 | 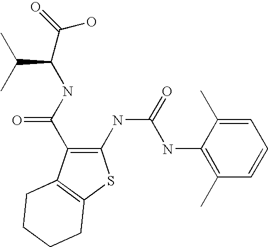 | N-{[2-({[(2,6- dimethylphenyl)amino]carbonyl}amino)-4,5,6,7- tetrahydro-1-benzothien-3- yl]carbonyl}-L-valine | 444.6 | 32.20 |
| 4 | 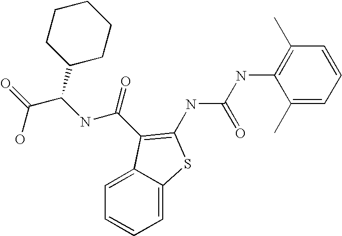 | (2S)-cyclohexyl({[2-({[(2,6- dimethylphenyl)amino]carbonyl}amino)-1-benzothien- 3-yl]carbonyl}amino)acetic acid | 480.4 | 0.73 |
| 5 | 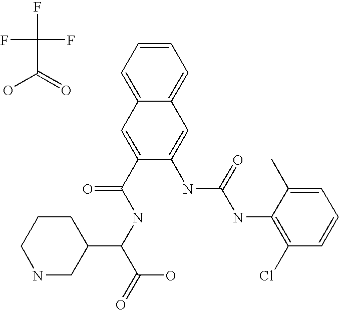 | {[3-({[(2-chloro-6- methylphenyl) amino]carbonyl}amino)-2- naphthoyl]amino}(piperidin-3-yl)acetic acid trifluoroacetate | 495.6 | 20.90 |
| 6 | 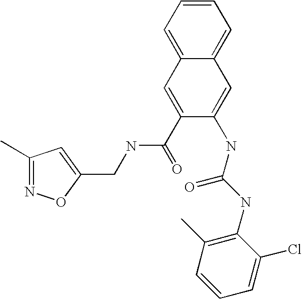 | 3-({[(2-chloro-6- methylphenyl) amino]carbonyl}amino)-N- [(3-methylisoxazol-5- yl)methyl]-2-naphthamide | 449.2 | 35.10 |
| 7 | 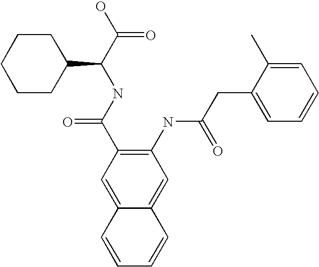 | (2S)-cyclohexyl{[(3-{[(2- methylphenyl) acetyl]amino}-2- naphthalenyl) carbonyl]amino}ethanoic acid | 459.6 | 17.00 |
| 8 | 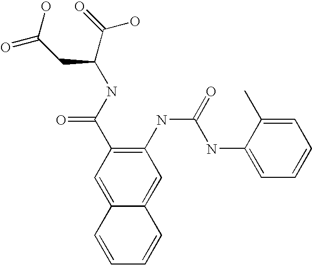 | N-[3-({[(2-methylphenyl) amino]carbonyl}amino)-2- naphthoyl]-L-aspartic acid | 436.4 | 3.91 |
| 9 | 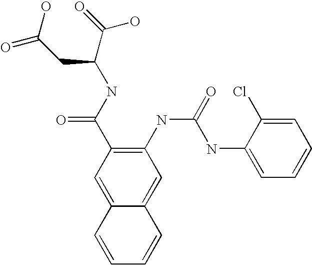 | N-[3-({[(2-chlorophenyl) amino]carbonyl}amino)-2- naphthoyl]-L-aspartic acid | 456.2 | 2.78 |
| 10 | 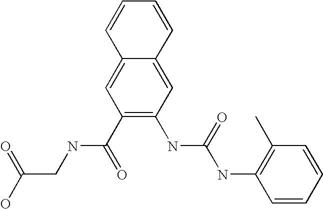 | N-[3-({[(2-methylphenyl) amino]carbonyl}amino)-2- naphthoyl]glycine | 378.2 | 6.50 |
| 11 | 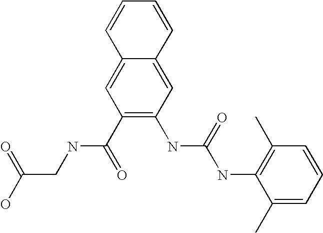 | N-[3-({[(2,6- dimethylphenyl)amino]carbonyl}amino)-2-naphthoyl]glycine | 392.2 | 0.09 |
| 12 | 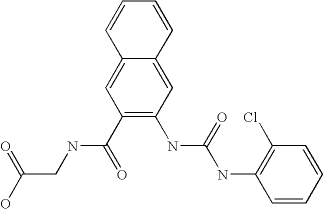 | N-[3-({[(2-chlorophenyl) amino]carbonyl}amino)-2- naphthoyl]glycine | 398.2 | 2.52 |
| 13 | 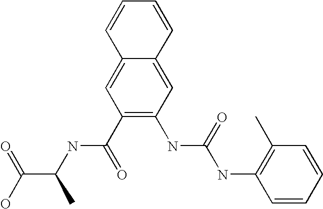 | N-[3-({[(2-methylphenyl)amino]carbonyl}amino)-2- naphthoyl]-L-alanine | 392.2 | 15.50 |
| 14 | 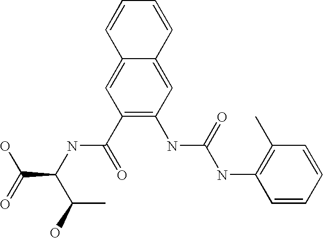 | N-[3-({[(2-methylphenyl)amino]carbonyl}amino)-2- naphthoyl]-L-threonine | 422.2 | 6.41 |
| 15 | 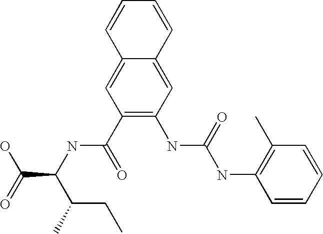 | N-[3-({[(2-methylphenyl)amino]carbonyl}amino)-2- naphthoyl]-L-isoleucine | 434.4 | 11.00 |
| 16 | 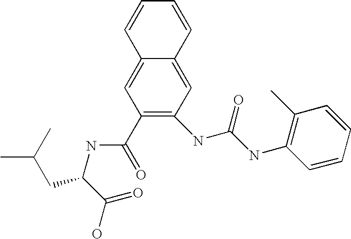 | N-[3-({[(2-methylphenyl)amino]carbonyl}amino)-2-naphthoyl]-L- leucine | 434.4 | 5.10 |
| 17 | 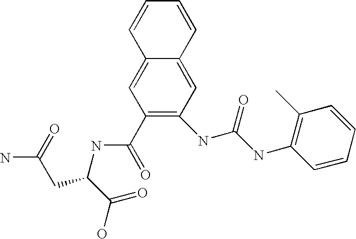 | N-[3-({[(2-methylphenyl)amino]carbonyl}amino)-2- naphthoyl]-L-asparagine | 435.4 | 4.72 |
| 18 | 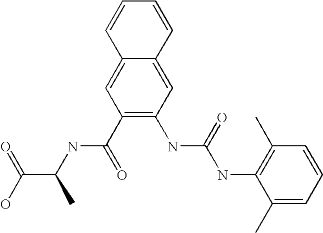 | N-[3-({[(2,6- dimethylphenyl)amino]carbonyl}amino)-2-naphthoyl]- L-alanine | 406.4 | 1.48 |
| 19 | 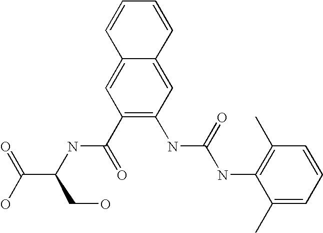 | N-[3-({[(2,6- dimethylphenyl)amino]carbonyl}amino)-2-naphthoyl]- L-serine | 422.2 | 2.00 |
| 20 | 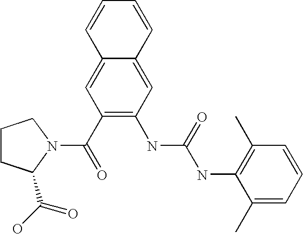 | 1-[3-({[(2,6- dimethylphenyl)amino]carbonyl}amino)-2-naphthoyl]- L-proline | 432.4 | 2.24 |
| 21 | 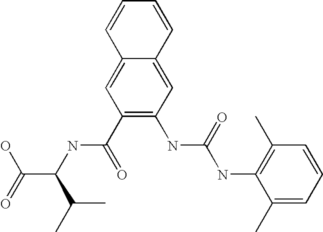 | N-[3-({[(2,6- dimethylphenyl)amino]carbonyl}amino)-2-naphthoyl]- L-valine | 434.4 | 1.96 |
| 22 | 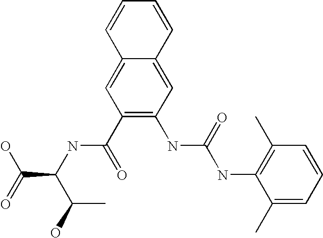 | N-[3-({[(2,6- dimethylphenyl)amino]carbonyl}amino)-2-naphthoyl]- L-threonine | 436.4 | 1.08 |
| 23 | 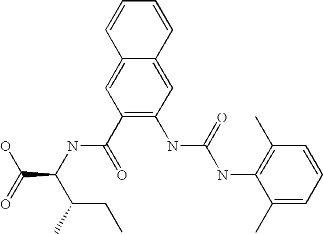 | N-[3-({[(2,6- dimethylphenyl)amino]carbonyl}amino)-2-naphthoyl]- L-isoleucine | 448.4 | 0.89 |
| 24 | 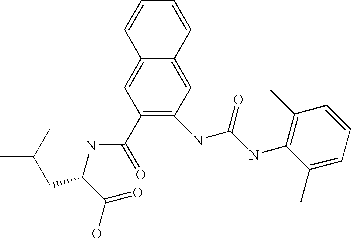 | N-[3-({[(2,6- dimethylphenyl)amino]carbonyl}amino)-2-naphthoyl]- L-leucine | 448.4 | 1.19 |
| 25 | 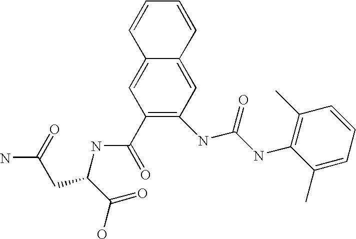 | N-[3-({[(2,6- dimethylphenyl)amino]carbonyl}amino)-2-naphthoyl]- L-asparagine | 469.2 | 0.67 |
| 26 | 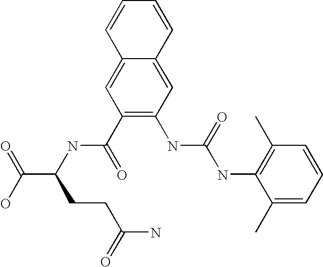 | N-[3-({[(2,6- dimethylphenyl)amino]carbonyl}amino)-2-naphthoyl]- L-glutamine | 463.4 | 3.01 |
| 27 | 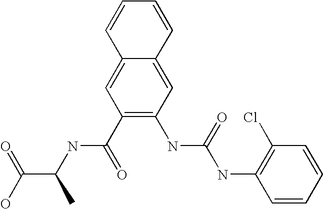 | N-[3-({[(2-chlorophenyl) amino]carbonyl}amino)-2- naphthoyl]-L-alanine | 412.2 | 13.50 |
| 28 | 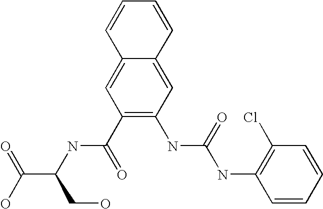 | N-[3-({[(2-chlorophenyl) amino]carbonyl}amino)-2- naphthoyl]-L-serine | 428.2 | 24.10 |
| 29 | 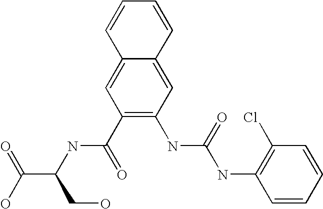 | N-[3-({[(2-chlorophenyl) amino]carbonyl}amino)-2- naphthoyl]-L-threonine | 442.2 | 7.26 |
| 30 | 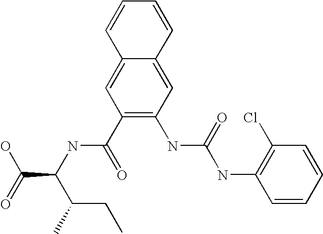 | N-[3-({[(2-chlorophenyl) amino]carbonyl}amino)-2- naphthoyl]-L-isoleucine | 454.2 | 22.10 |
| 31 | 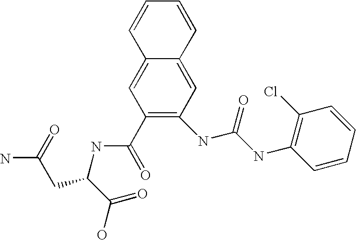 | N-[3-({[(2-chlorophenyl) amino]carbonyl}amino)-2- naphthoyl]-L-asparagine | 455.2 | 11.40 |
| 32 | 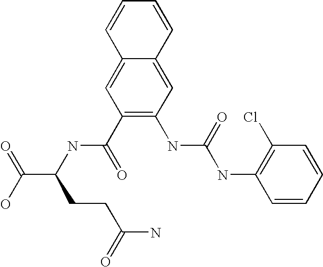 | N-[3-({[(2-chlorophenyl) amino]carbonyl}amino)-2- naphthoyl]-L-glutamine | 469.2 | 41.00 |
| 33 | 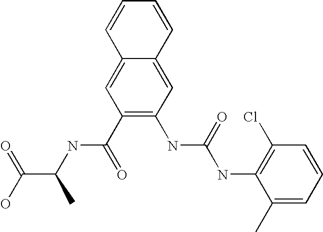 | N-[3-({[(2-chloro-6-methylphenyl) amino]carbonyl}amino)-2-naphthoyl]-L- alanine | 426.2 | 0.52 |
| 34 | 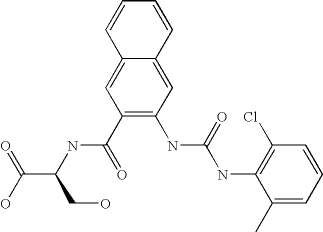 | N-[3-({[(2-chloro-6- methylphenyl) amino]carbonyl}amino)-2- naphthoyl]-L-serine | 442.2 | 0.64 |
| 35 | 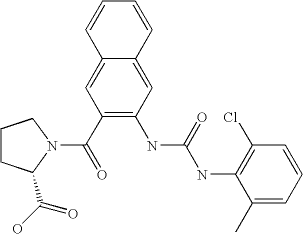 | 1-[3-({[(2-chloro-6- methylphenyl) amino]carbonyl}amino)-2- naphthoyl]-L-proline | 452.2 | 0.58 |
| 36 | 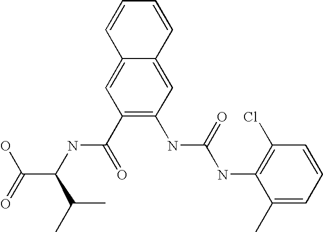 | N-[3-({[(2-chloro-6- methylphenyl) amino]carbonyl}amino)-2- naphthoyl]-L-valine | 454.2 | 1.71 |
| 37 | 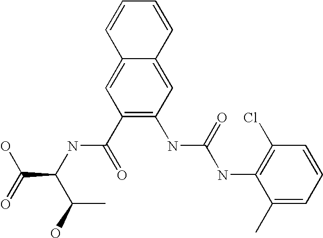 | N-[3-({[(2-chloro-6- methylphenyl) amino]carbonyl}amino)-2- naphthoyl]-L-threonine | 456.2 | 0.43 |
| 38 | 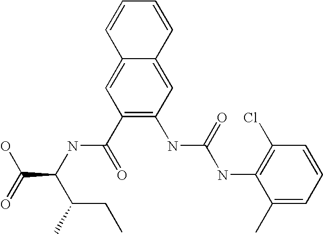 | N-[3-({[(2-chloro-6- methylphenyl) amino]carbonyl}amino)-2- naphthoyl]-L-isoleucine | 468.2 | 0.28 |
| 39 | 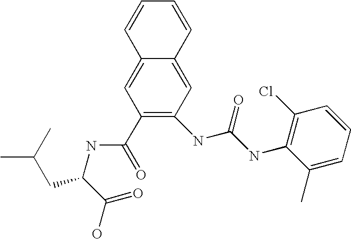 | N-[3-({[(2-chloro-6- methylphenyl) amino]carbonyl}amino)-2- naphthoyl]-L-leucine | 467.2 | 0.60 |
| 40 | 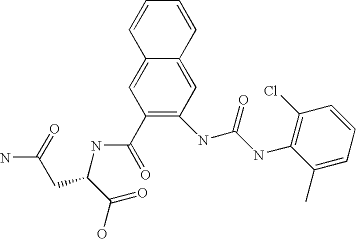 | N-[3-({[(2-chloro-6- methylphenyl) amino]carbonyl}amino)-2- naphthoyl]-L-asparagine | 469.2 | 0.42 |
| 41 | 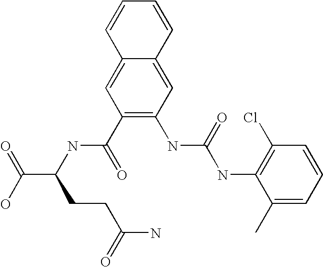 | N-[3-({[(2-chloro-6- methylphenyl) amino]carbonyl}amino)-2- naphthoyl]-L-glutamine | 483.2 | 1.32 |
| 42 | 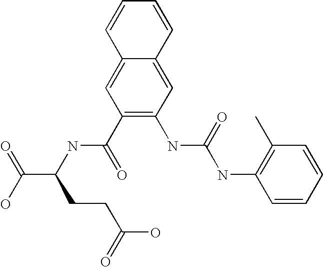 | N-[3-({[(2-methylphenyl) amino]carbonyl}amino)-2- naphthoyl]-L-glutamic acid | 450.2 | 28.30 |
| 43 | 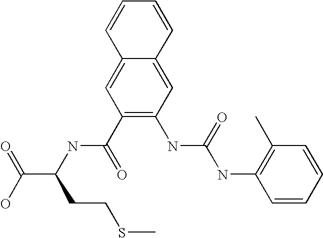 | N-[3-({[(2-methylphenyl) amino]carbonyl}amino)-2- naphthoyl]-L-methionine | 452.2 | 18.90 |
| 44 | 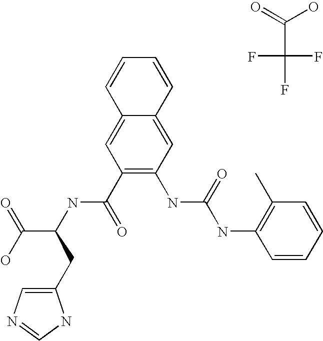 | N-[3-({[(2-methylphenyl) amino]carbonyl}amino)-2- naphthoyl]-L-histidine trifluoroacetate | 458.2 | 5.18 |
| 45 | 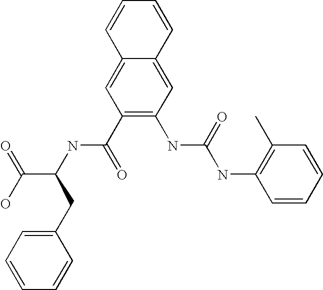 | N-[3-({[(2-methylphenyl) amino]carbonyl}amino)-2- naphthoyl]-L-phenylalanine | 468.2 | 16.90 |
| 46 | 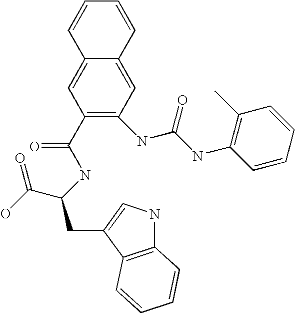 | N-[3-({[(2-methylphenyl) amino]carbonyl}amino)-2- naphthoyl]-L-tryptophan | 507.4 | 3.73 |
| 47 | 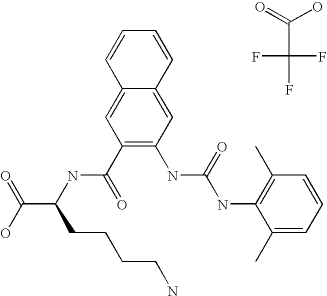 | N-[3-({[(2,6- dimethylphenyl)amino]carbonyl}amino)-2-naphthoyl]- L-lysine trifluoroacetate | 463.4 | 8.06 |
| 48 | 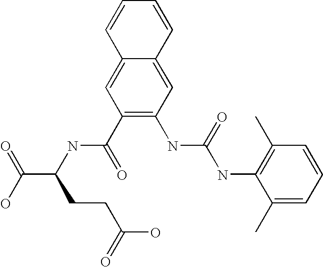 | N-[3-({[(2,6- dimethylphenyl)amino]carbonyl}amino)-2-naphthoyl]- L-glutamic acid | 464.2 | 1.73 |
| 49 | 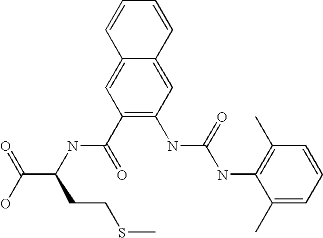 | N-[3-({[(2,6- dimethylphenyl)amino]carbonyl}amino)-2-naphthoyl]- L-methionine | 466.4 | 1.67 |
| 50 | 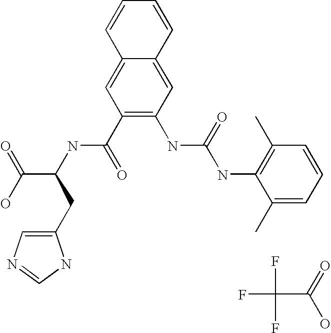 | N-[3-({[(2,6- dimethylphenyl)amino]carbonyl}amino)-2-naphthoyl]- L-histidine trifluoroacetate | 472.4 | 2.30 |
| 51 | 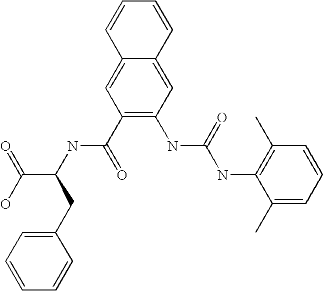 | N-[3-({[(2,6- dimethylphenyl)amino]carbonyl}amino)-2-naphthoyl]- L-phenylalanine | 482.4 | 0.68 |
| 52 | 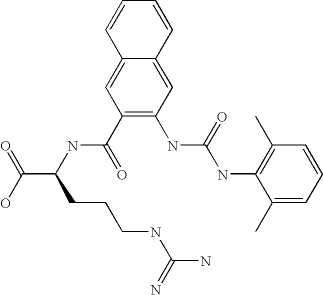 | N-[3-({[(2,6- dimethylphenyl)amino]carbonyl}amino)-2-naphthoyl]- L-arginine | 491.4 | 4.64 |
| 53 | 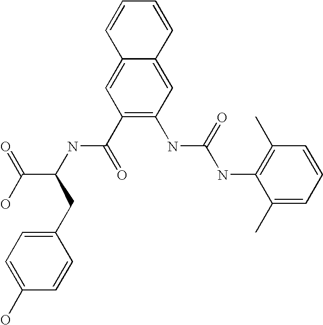 | N-[3-({[(2,6- dimethylphenyl)amino]carbonyl}amino)-2-naphthoyl]- L-tyrosine | 498.4 | 0.36 |
| 54 | 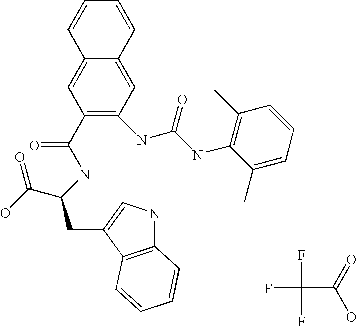 | N-[3-({[(2,6- dimethylphenyl)amino]carbonyl}amino)-2-naphthoyl]- L-tryptophan trifluoroacetate | 521.4 | 0.51 |
| 55 | 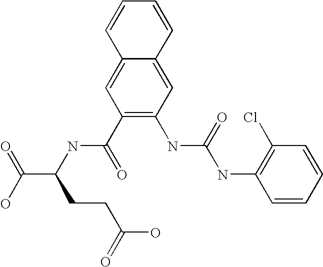 | N-[3-({[(2-chlorophenyl) amino]carbonyl}amino)-2- naphthoyl]-L-glutamic acid | 470.2 | 10.40 |
| 56 | 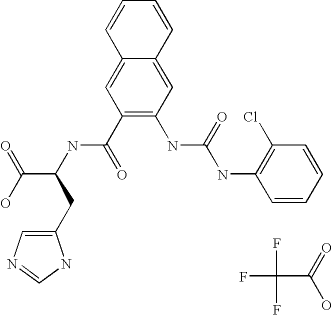 | N-[3-({[(2-chlorophenyl) amino]carbonyl)amino)-2- naphthoyl]-L-histidine trifluoroacetate | 478.2 | 3.08 |
| 57 | 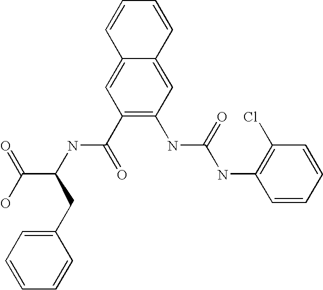 | N-[3-({[(2-chlorophenyl) amino]carbonyl}amino)-2- naphthoyl]-L-phenylalanine | 488.4 | 5.21 |
| 58 | 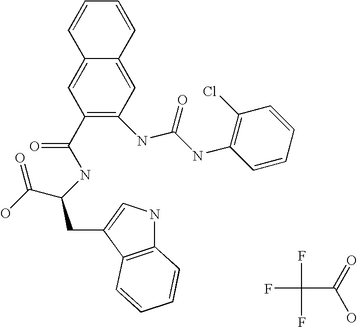 | N-[3-({[(2-chlorophenyl) amino]carbonyl}amino)-2- naphthoyl]-L-tryptophan trifluoroacetate | 527.4 | 4.70 |
| 59 | 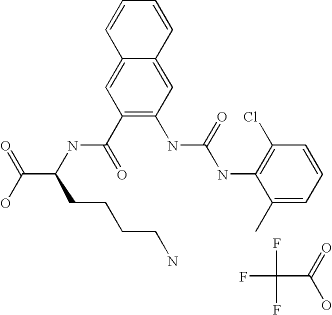 | N-[3-({[(2-chloro-6- methylphenyl) amino]carbonyl}amino)-2- naphthoyl]-L-lysine trifluoroacetate | 483.4 | 2.99 |
| 60 | 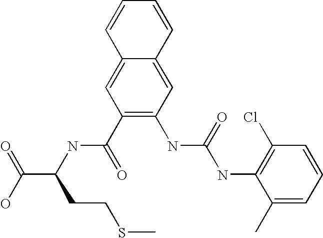 | N-[3-({[(2-chloro-6- methylphenyl) amino]carbonyl}amino)-2- naphthoyl]-L-methionine | 452.2 | 0.64 |
| 61 | 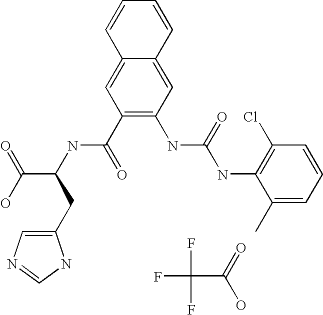 | N-[3-({[(2-chloro-6- methylphenyl) amino]carbonyl}amino)-2- naphthoyl]-L-histidine trifluoroacetate | 492.2 | 0.45 |
| 62 | 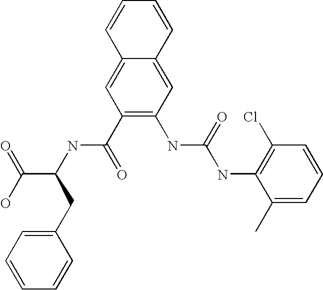 | N-[3-({[(2-chloro-6- methylphenyl) amino]carbonyl}amino)-2- naphthoyl]-L-phenylalanine | 502.4 | 0.26 |
| 63 | 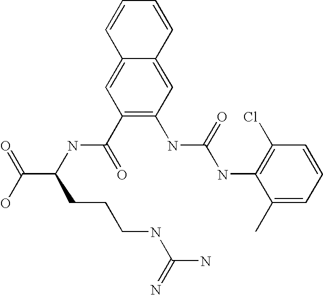 | N-[3-({[(2-chloro-6- methylphenyl) amino]carbonyl}amino)-2- naphthoyl]-L-arginine | 511.4 | 2.73 |
| 64 | 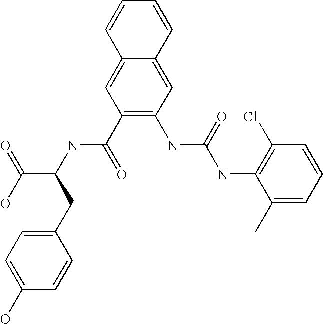 | N-[3-({[(2-chloro-6- methylphenyl) amino]carbonyl}amino)-2- naphthoyl]-L-tyrosine | 518.4 | 0.45 |
| 65 | 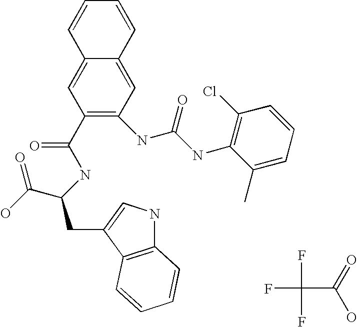 | N-[3-({[(2-chloro-6- methylphenyl) amino]carbonyl}amino)-2- naphthoyl]-L-tryptophan trifluoroacetate | 541.4 | 0.27 |
| 66 | 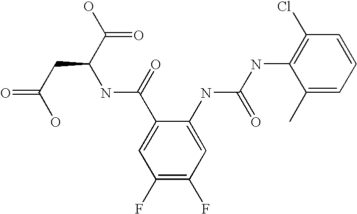 | N-[2-({[(2-chloro-6- methylphenyl) amino]carbonyl}amino)- 4,5-difluorobenzoyl]-L- aspartic acid | 456.4 | 7.16 |
| 67 | 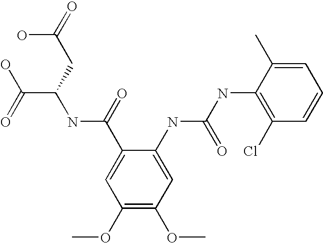 | N-[2-({[(2-chloro-6- methylphenyl) amino]carbonyl}amino)- 4,5-dimethoxy-benzoyl]-L- aspartic acid | 480.4 | 19.80 |
| 68 | 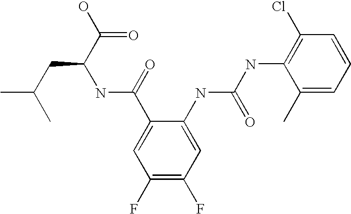 | N-[2-({[(2-chloro-6 methylphenyl) amino]carbonyl}amino)- 4,5-difluorobenzoyl]-L- leucine | 454.4 | 7.54 |
| 69 | 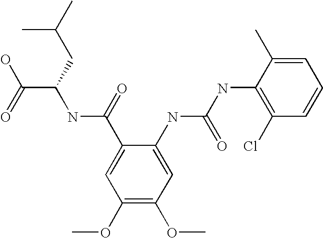 | N-[2-({[(2-chloro-6- methylphenyl) amino]carbonyl}amino)- 4,5-dimethoxy-benzoyl]-L- leucine | 478.4 | 13.80 |
| 70 | 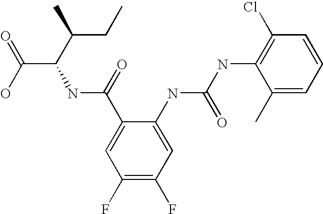 | N-[2-({[(2-chloro-6- methylphenyl) amino]carbonyl}amino)- 4,5-difluorobenzoyl]-L- isoleucine | 454.4 | 5.97 |
| 71 | 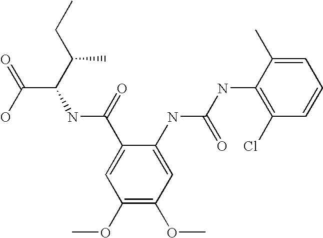 | N-[2-({[(2-chloro-6- methylphenyl) amino]carbonyl}amino)- 4,5-dimethoxy-benzoyl]-L- isoleucine | 478.4 | 22.30 |
| 72 | 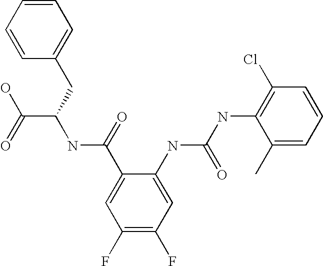 | N-[2-({[(2-chloro-6- methylphenyl) amino]carbonyl}amino)- 4,5-difluorobenzoyl]-L- phenylalanine | 488.4 | 10.90 |
| 73 | 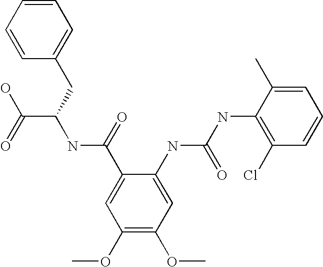 | N-[2-({[(2-chloro-6- methylphenyl) amino]carbonyl}amino)- 4,5-dimethoxy-benzoyl]-L- phenylalanine | 512.4 | 11.60 |
| 74 | 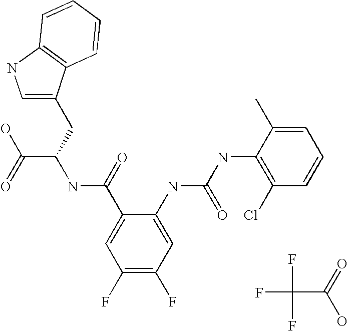 | N-[2-({[(2-chloro-6- methylphenyl) amino]carbonyl}amino)- 4,5-difluorobenzoyl]-L- tryptophan trifluoroacetate | 527.4 | 6.58 |
| 75 | 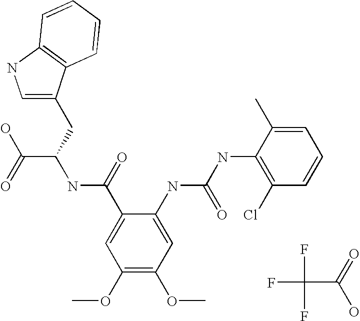 | N-[2-({[(2-chloro-6- methylphenyl) amino]carbonyl}amino)- 4,5-dimethoxy-benzoyl]-L- tryptophan trifluoroacetate | 551.4 | 0.76 |
| 76 | 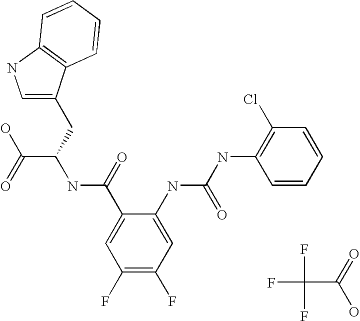 | N-[2-({[(2-chlorophenyl) amino]carbonyl}amino)- 4,5-difluorobenzoyl]-L- tryptophan trifluoroacetate | 513.4 | 3.74 |
| 77 | 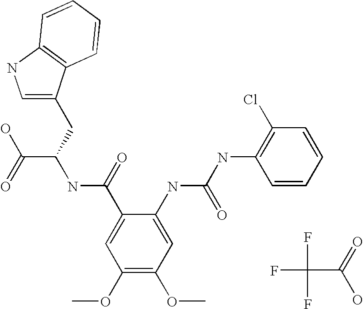 | N-[2-({[(2-chlorophenyl) amino]carbonyl}amino)- 4,5-dimethoxy-benzoyl]-L- tryptophan trifluoroacetate | 537.2 | 9.05 |
| 78 | 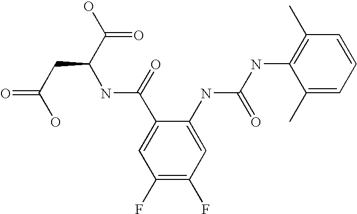 | N-[2-({[(2,6- dimethylphenyl)amino]carbonyl}amino)-4,5- difluorobenzoyl]-L-aspartic acid | 436.2 | 9.11 |
| 79 | 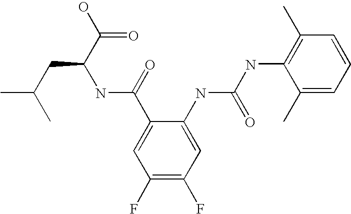 | N-[2-({[(2,6- dimethylphenyl)amino]carbonyl}amino)-4,5- difluorobenzoyl]-L-leucine | 434.4 | 18.70 |
| 80 | 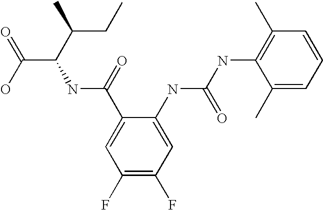 | N-[2-({[(2,6- dimethylphenyl)amino]carbonyl}amino)-4,5- difluorobenzoyl]-L- isoleucine | 434.4 | 8.73 |
| 81 | 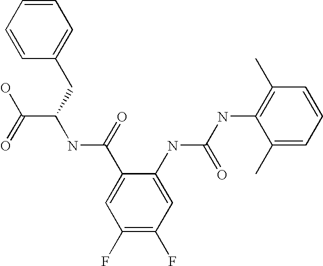 | N-[2-({[(2,6- dimethylphenyl)amino]carbonyl}amino)-4,5- difluorobenzoyl]-L- phenylalanine | 468.4 | 14.70 |
| 82 | 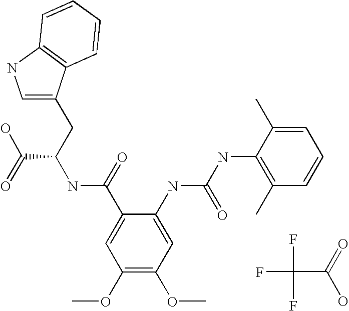 | N-[2-({[(2,6- dimethylphenyl)amino]carbonyl}amino)-4,5- dimethoxy-benzoyl]-L- tryptophan trifluoroacetate | 531.4 | 9.41 |
| 83 | 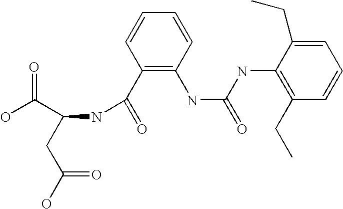 | N-[2-({[(2,6-diethylphenyl) amino]carbonyl}amino)benzoyl]- L-aspartic acid | 428.0 | 34.15 |
| 84 | 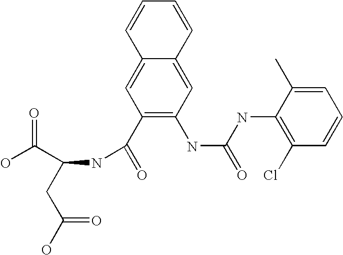 | N-[3-({[(2-chloro-6- methylphenyl) amino]carbonyl}amino)-2- naphthoyl]-L-aspartic acid | 470.2 | 0.39 |
| 85 | 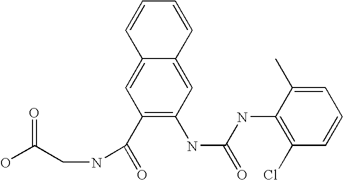 | N-[3-({[(2-chloro-6- methylphenyl) amino]carbonyl}amino)-2- naphthoyl]glycine | 412.2 | 0.57 |
| 86 | 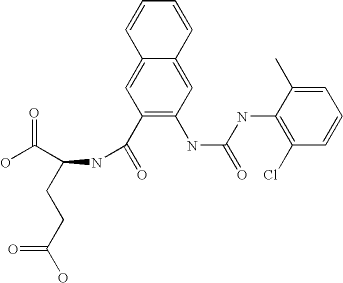 | N-[3-({[(2-chloro-6- methylphenyl) amino]carbonyl}amino)-2- naphthoyl]-L-glutamic acid | 464.2 | 0.70 |
| 87 | 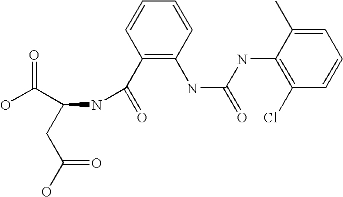 | N-[2-({[(2-chloro-6- methylphenyl) amino]carbonyl}amino) benzoyl]-L-aspartic acid | 419.8 | 24.50 |
| 88 | 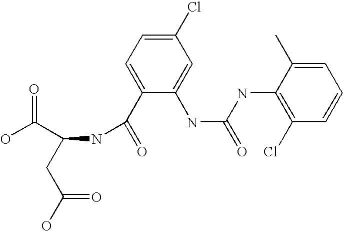 | N-[4-chloro-2-({[(2-chloro- 6-methylphenyl) amino]carbonyl}amino) benzoyl]-L-aspartic acid | 454.0 | 5.20 |
| 89 | 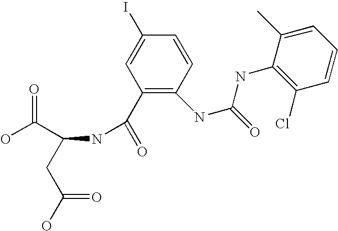 | N-[2-({[(2-chloro-6- methylphenyl) amino]carbonyl}amino)-5- iodobenzoyl]-L-aspartic acid | 456.0 | 17.45 |
| 90 | 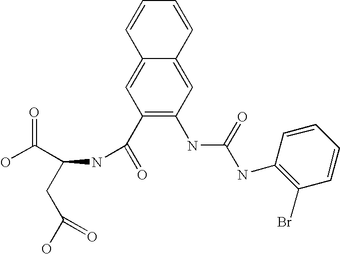 | N-[3-({[(2-bromophenyl) amino]carbonyl}amino)-2- naphthoyl]-L-aspartic acid | 500.1 | 22.95 |
| 91 | 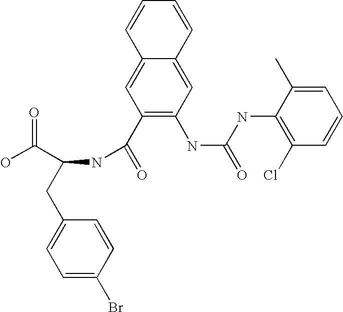 | 4-bromo-N-[3-({[(2-chloro- 6-methylphenyl) amino]carbonyl}amino)-2- naphthoyl]-L-phenylalanine | 580.0 | 7.88 |
| 92 | 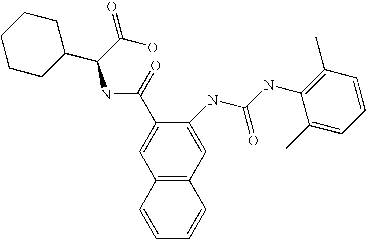 | (2S)-cyclohexyl{[3-({[(2,6- dimethylphenyl)amino]carbonyl}amino)-2- naphthoyl]amino}acetic acid | 474.4 | 0.12 |
| 93 | 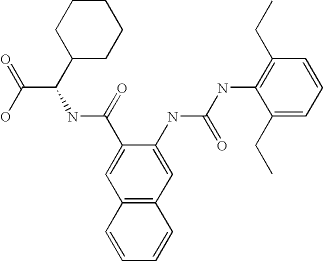 | (2S)-cyclohexyl({[3-({[(2,6- diethylphenyl) amino]carbonyl}amino)-2- naphthalenyl]carbonyl}amino) ethanoic acid | 502.4 | 0.59 |
| 94 | 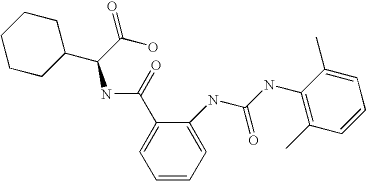 | (2S)-cyclohexyl{[2-({[(2,6- dimethylphenyl)amino]carbonyl}amino)benzoyl]amino}acetic acid | 424.2 | 0.53 |
| 95 | 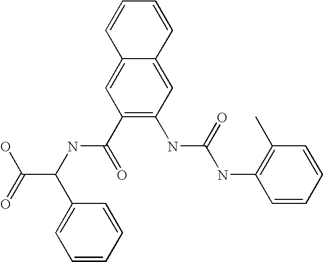 | {[3-({[(2-methylphenyl) amino]carbonyl}amino)-2- naphthoyl]amino}(phenyl) acetic acid | 454.2 | 9.89 |
| 96 | 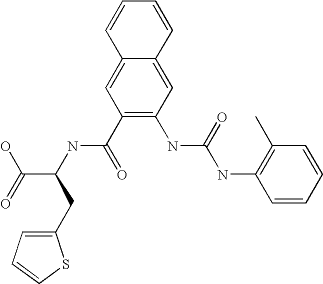 | N-{[3-({[(2-methylphenyl) amino]carbonyl}amino)-2- naphthalenyl]carbonyl}-3-(2-thienyl)-L- alanine | 474.4 | 5.07 |
| 97 | 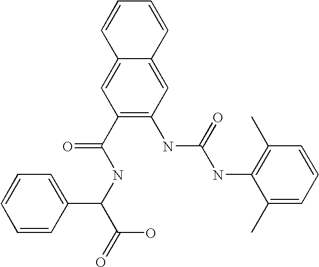 | {[3-({[(2,6- dimethylphenyl)amino]carbonyl}amino)-2-naphthoyl]amino}(phenyl) acetic acid | 468.2 | 0.66 |
| 98 | 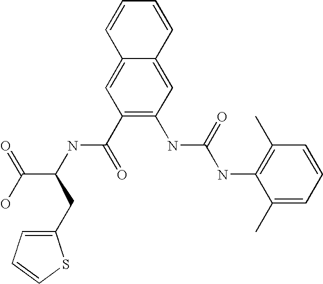 | N-{[3-({[(2,6- dimethylphenyl)amino]carbonyl}amino)-2- naphthalenyl]carbonyl}-3-(2-thienyl)-L- alanine | 488.4 | 0.54 |
| 99 | 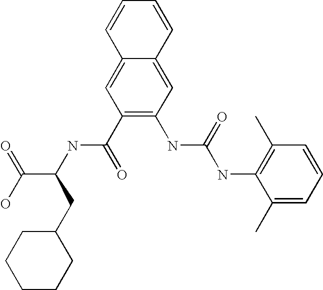 | 3-cyclohexyl-N-[3-({[(2,6- dimethylphenyl)amino]carbonyl}amino)-2-naphthoyl]- L-alanine | 488.6 | 0.49 |
| 100 | 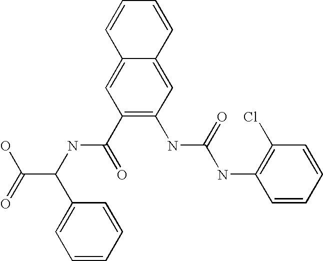 | {[3-({[(2-chlorophenyl) amino]carbonyl}amino)-2- naphthoyl]amino}(phenyl) acetic acid | 474.4 | 7.63 |
| 101 | 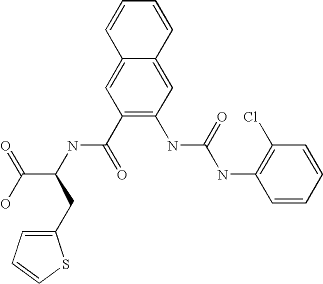 | N-{[3-({[(2-chlorophenyl) amino]carbonyl}amino)-2- naphthalenyl]carbonyl}-3-(2-thienyl)-L- alanine | 494.2 | 2.78 |
| 102 | 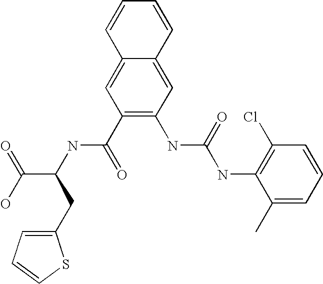 | N-{[3-({[(2-chloro-6- methylphenyl) amino]carbonyl}amino)-2- naphthalenyl]carbonyl}-3-(2-thienyl)-L- alanine | 508.4 | 0.36 |
| 103 | 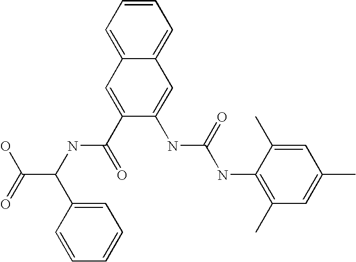 | phenyl({[3-({[(2,4,6- trimethylphenyl)amino]carbonyl}amino)-2- naphthalenyl]carbonyl}amino)acetic acid | 482.2 | 0.06 |
| 104 | 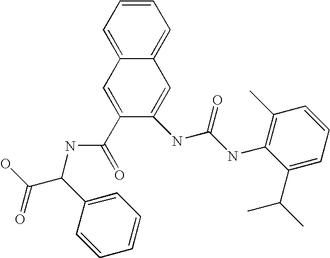 | {[3-({[(2-isopropyl-6- methylphenyl) amino]onyl}amino)-2- naphthoyl]amino}(phenyl) acetic acid | 496.4 | 1.04 |
| 105 | 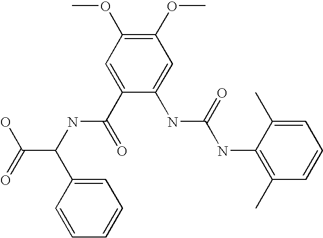 | {[2-({[(2,6- dimethylphenyl)amino]carbonyl}amino)-4,5- dimethoxybenzoyl]amino}(phenyl)acetic acid | 478.2 | 19.60 |
| 106 | 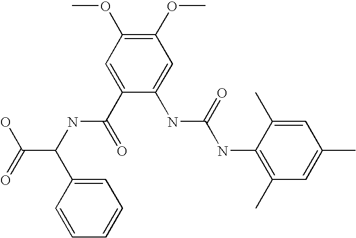 | [(2- {[(mesitylamino)carbonyl]amino}-4,5-dimethoxy- benzoyl)amino](phenyl)acetic acid | 492.4 | 4.23 |
| 107 | 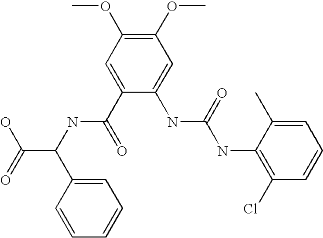 | {[2-({[(2-chloro-6- methylphenyl) amino]carbonyl}amino)- 4,5-dimethoxy- benzoyl]amino}(phenyl)acetic acid | 498.2 | 12.20 |
| 108 | 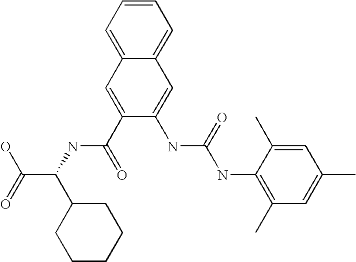 | (2R)-cyclohexyl[(3- {[(mesitylamino)carbonyl]amino}-2-naphthoyl) amino]acetic acid | 488.4 | 3.29 |
| 109 | 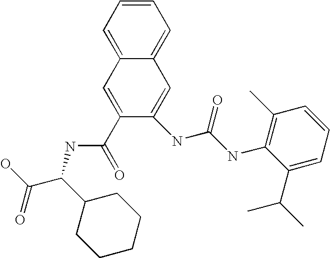 | (2R)-cyclohexyl{[3-({[(2- isopropyl-6-methylphenyl) amino]carbonyl}amino)-2- naphthoyl]amino}acetic acid | 502.4 | 19.80 |
| 110 | 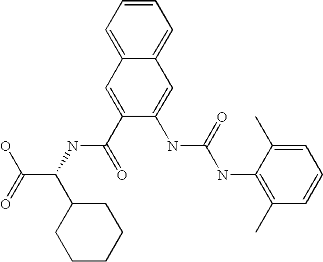 | (2R)-cyclohexyl{[3-({[(2,6- dimethylphenyl)amino]carbonyl}amino)-2-naphthoyl]amino}acetic acid | 474.4 | 12.00 |
| 111 | 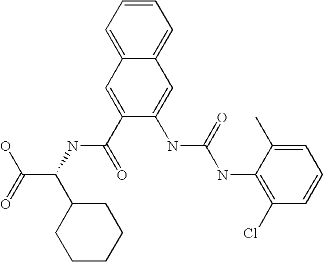 | (2R)-{[3-({[(2-chloro-6- methylphenyl) amino]carbonyl}amino)-2- naphthoyl]amino}(cyclohexyl) acetic acid | 494.2 | 4.15 |
| 112 | 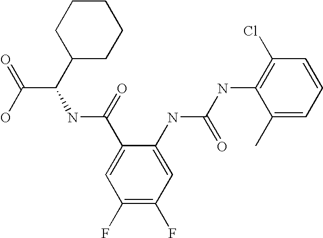 | (2S)-{[2-({[(2-chloro-6- methylphenyl) amino]carbonyl}amino)- 4,5-difluorobenzoyl]amino}(cyclohexyl) acetic acid | 480.4 | 0.86 |
| 113 | 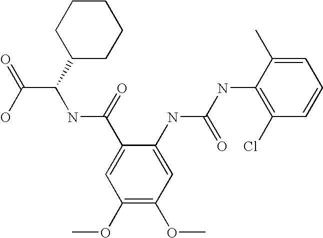 | (2S)-{[2-({[(2-chloro-6- methylphenyl) amino]carbonyl}amino)- 4,5-dimethoxy- benzoyl]amino}(cyclohexyl) acetic acid | 504.4 | 7.04 |
| 114 | 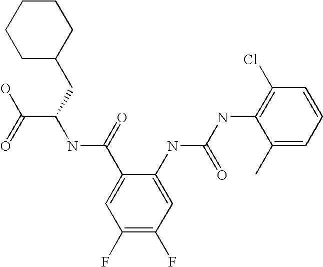 | N-[2-({[(2-chloro-6- methylphenyl) amino]carbonyl}amino)- 4,5-difluorobenzoyl]-3- cyclohexyl-L-alanine | 494.4 | 18.70 |
| 115 | 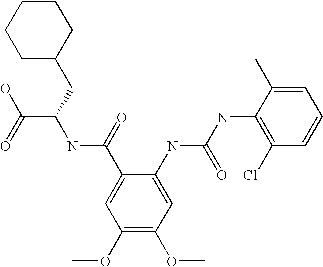 | N-[2-({[(2-chloro-6- methylphenyl) amino]carbonyl}amino)- 4,5-dimethoxy-benzoyl]-3- cyclohexyl-L-alanine | 518.4 | 13.00 |
| 116 | 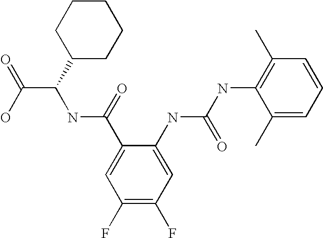 | (2S)-cyclohexyl{[2-({[(2,6- dimethylphenyl)amino]carbonyl}amino)-4,5- difluorobenzoyl]amino}acetic acid | 460.4 | 1.63 |
| 117 | 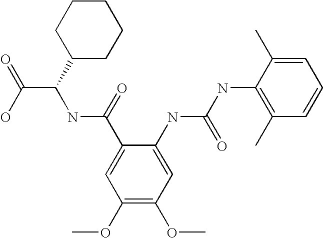 | (2S)-cyclohexyl{[2-({[(2,6- dimethylphenyl)amino]carbonyl}amino)-4,5- dimethoxy-benzoyl]amino}acetic acid | 484.4 | 14.70 |
| 118 | 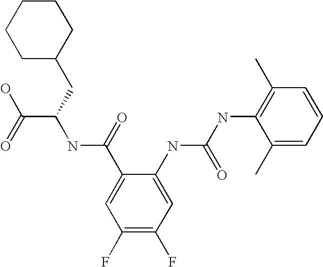 | 3-cyclohexyl-N-[2-({[(2,6- dimethylphenyl)amino]carbonyl}amino)-4,5- difluorobenzoyl]-L-alanine | 474.4 | 15.70 |
| 119 | 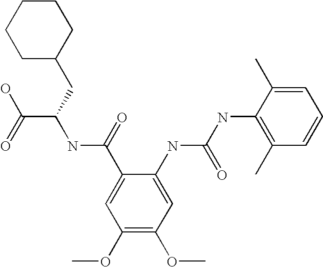 | 3-cyclohexyl-N-[2-({[(2,6- dimethylphenyl)amino]carbonyl}amino)-4,5- dimethoxy-benzoyl]-L- alanine | 498.4 | 13.60 |
| 120 | 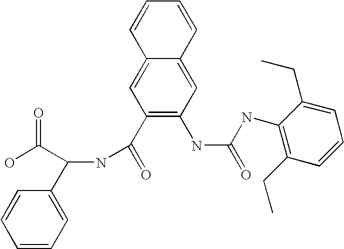 | {[3-({[(2,6-diethylphenyl) amino]carbonyl}amino)-2- naphthoyl]amino}(phenyl) acetic acid | 496.0 | 24.55 |
| 121 | 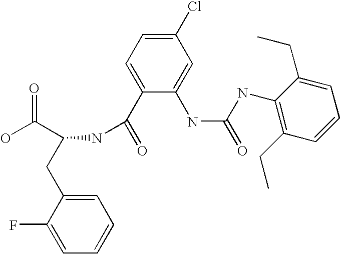 | N-[4-chloro-2-({[(2,6- diethylphenyl) amino]carbonyl}amino) benzoyl]-2-fluoro-D- phenylalanine | 512.2 | 3.44 |
| 122 | 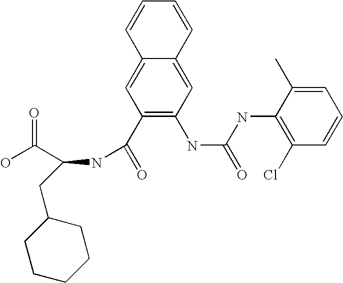 | N-[3-({[(2-chloro-6- methylphenyl) amino]carbonyl}amino)-2- naphthoyl]-3-cyclohexyl-L- alanine | 508.4 | 2.23 |
| 123 | 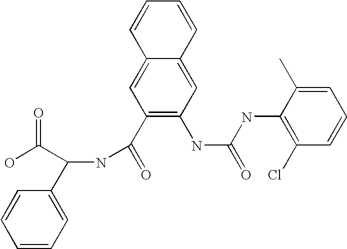 | ({[3-({[(2-chloro-6- methylphenyl) amino]carbonyl}amino)-2- naphthalenyl]carbonyl}amino)(phenyl) acetic acid | 488.4 | 0.27 |
| 124 | 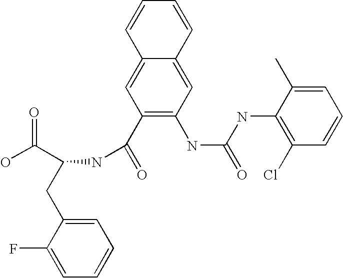 | N-[3-({[(2-chloro-6- methylphenyl) amino]carbonyl}amino)-2- naphthoyl]-2-fluoro-D- phenylalanine | 520.0 | 5.82 |
| 125 | 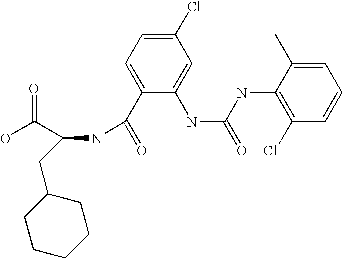 | N-[4-chloro-2-({[(2-chloro- 6-methylphenyl) amino]carbonyl}amino) benzoyl]-3-cyclohexyl-L- alanine | 492.0 | 28.60 |
| 126 | 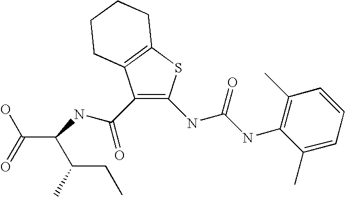 | N-{[2-({[(2,6- dimethylphenyl)amino]carbonyl}amino)-4,5,6,7- tetrahydro-1-benzothien-3- yl]carbonyl}-L-isoleucine | 458.2 | 13.90 |
| 127 | 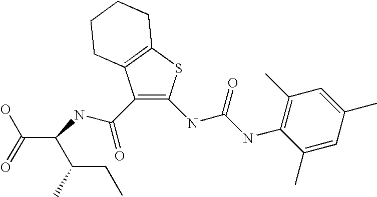 | N-[(2- {[(mesitylamino)carbonyl]amino}-4,5,6,7-tetrahydro-1- benzothien-3-yl)carbonyl]- L-isoleucine | 472.4 | 18.20 |
| 128 | 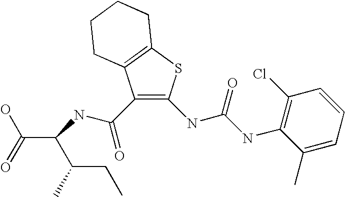 | N-{[2-({[(2-chloro-6- methylphenyl) amino]carbonyl}amino)- 4,5,6,7-tetrahydro-1- benzothien-3-yl]carbonyl}- L-isoleucine | 479 | 14.80 |
| 129 | 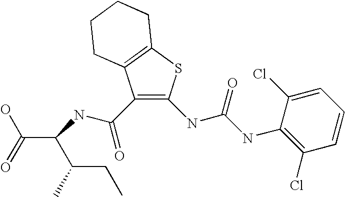 | N-{[2-({[(2,6- dichlorophenyl) amino]carbonyl}amino)- 4,5,6,7-tetrahydro-1- benzothien-3-yl]carbonyl}- L-isoleucine | 499.2 | 7.97 |
| 130 | 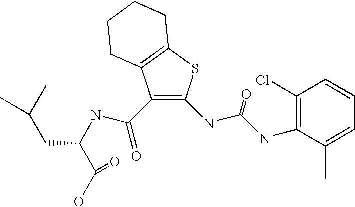 | N-{[2-({[(2-chloro-6- methylphenyl) amino]carbonyl}amino)- 4,5,6,7-tetrahydro-1- benzothien-3-yl]carbonyl}- L-leucine | 479.2 | 18.00 |
| 131 | 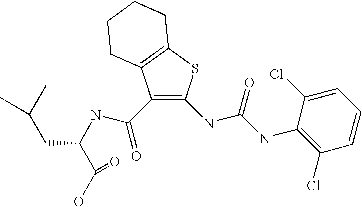 | N-{[2-({[(2,6- dichlorophenyl) amino]carbonyl}amino)- 4,5,6,7-tetrahydro-1- benzothien-3-yl]carbonyl}- L-leucine | 499.4 | 9.96 |
| 132 | 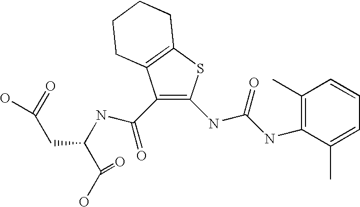 | N-{[2-({[(2,6- dimethylphenyl)amino]carbonyl}amino)-4,5,6,7- tetrahydro-1-benzothien-3- yl]carbonyl}-L-aspartic acid | 460.2 | 5.25 |
| 133 | 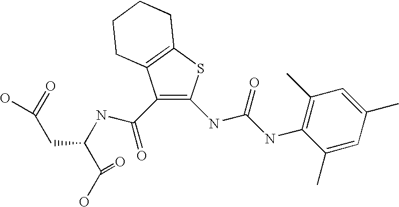 | N-[(2- {[(mesitylamino)carbonyl]amino}-4,5,6,7-tetrahydro-1- benzothien-3-yl)carbonyl]- L-aspartic acid | 474.4 | 2.38 |
| 134 | 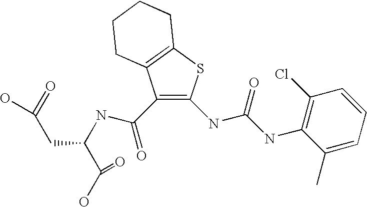 | N-{[2-({[(2-chloro-6- methylphenyl) amino]carbonyl}amino)- 4,5,6,7-tetrahydro-1- benzothien-3-yl]carbonyl}- L-aspartic acid | 480.4 | 3.66 |
| 135 | 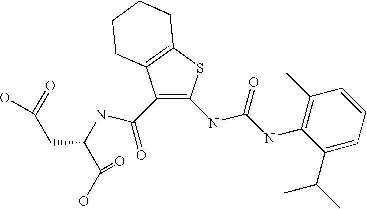 | N-{[2-({[(2-isopropyl-6- methylphenyl) amino]carbonyl}amino)- 4,5,6,7-tetrahydro-1- benzothien-3-yl]carbonyl}- L-aspartic acid | 488.2 | 17.90 |
| 136 | 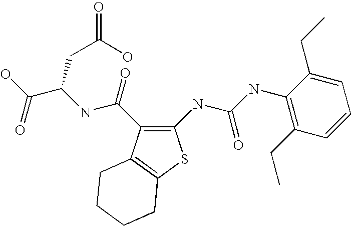 | N-{[2-({[(2,6-diethylphenyl) amino]carbonyl}amino)- 4,5,6,7-tetrahydro-1- benzothien-3-yl]carbonyl}- L-aspartic acid | 488.6 | 4.58 |
| 137 | 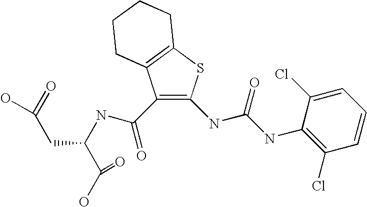 | N-{[2-({[2,6- dichlorophenyl) amino]carbonyl}amino)- 4,5,6,7-tetrahydro-1- benzothien-3-yl]carbonyl}- L-aspartic acid | 501.4 | 4.15 |
| 138 | 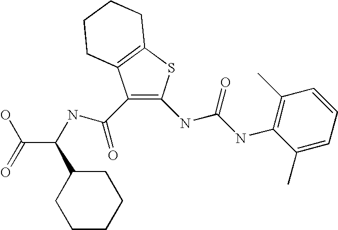 | (2S)-cyclohexyl({[2-({[(2,6- dimethylphenyl)amino]carbonyl}amino)-4,5,6,7- tetrahydro-1-benzothien-3- yl]carbonyl}amino)acetic acid | 484.4 | 5.56 |
| 139 | 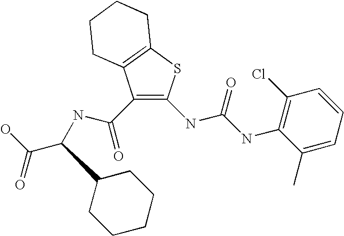 | (2S)-({[2-({[(2-chloro-6- methylphenyl) amino]carbonyl}amino)- 4,5,6,7-tetrahydro-1- benzothien-3-yl]carbonyl}amino) (cyclohexyl) acetic acid | 505.2 | 3.26 |
| 140 | 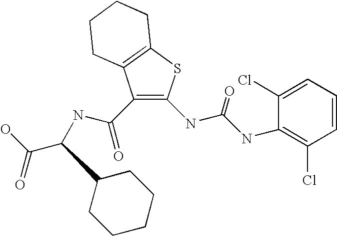 | (2S)-cyclohexyl({[2-({[(2,6- dichlorophenyl) amino]carbonyl}amino)- 4,5,6,7-tetrahydro-1- benzothien-3-yl]carbonyl}amino) acetic acid | 525.2 | 5.50 |
| 141 | 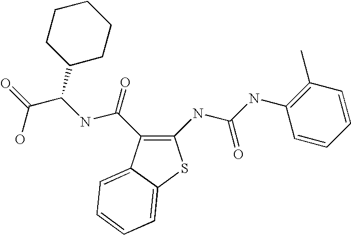 | (2S)-cyclohexyl({[2-({[(2- methylphenyl) amino]carbonyl}amino)-1- benzothien-3-yl]carbonyl}amino) acetic acid | 466.4 | 8.71 |
| 142 | 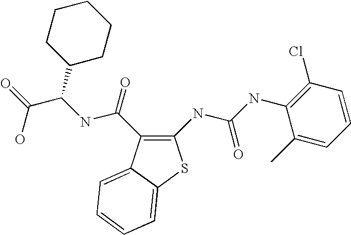 | (2S)-({[2-({[(2-chloro-6- methylphenyl) amino]carbonyl}amino)-1- benzothien-3-yl]carbonyl}amino) (cyclohexyl) acetic acid | 500.2 | 1.51 |
| 143 | 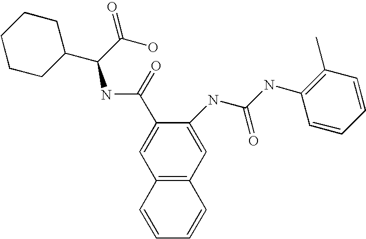 | (2S)-cyclohexyl{[3-({[(2- methylphenyl) amino]carbonyl}amino)-2- naphthoyl]amino}acetic acid | 460.8 | 30.34 |
| 144 | 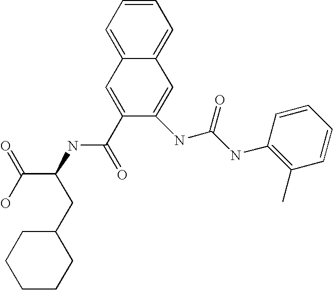 | 3-cyclohexyl-N-{[3-({[3-(2- methylphenyl) amino]carbonyl}amino)-2- naphthalenyl]carbonyl}-L-alanine | 474.6 | 16.80 |
| 145 | 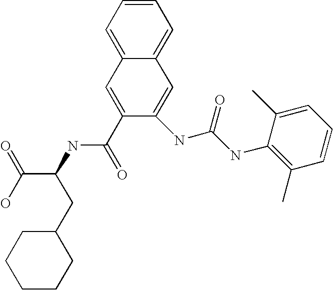 | 3-cyclohexyl-N-{[3-({[(2,6- dimethylphenyl)amino]carbonyl}amino)-2- naphthalenyl]carbonyl}-L-alanine | 488.8 | 0.59 |
| 146 | 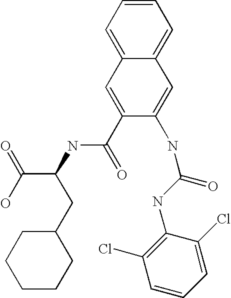 | 3-cyclohexyl-N-{[3-({[(2,6- dichlorophenyl) amino]carbonyl}amino)-2- naphthalenyl]carbonyl}-L-alanine | 528.4 | 0.54 |
| 147 | 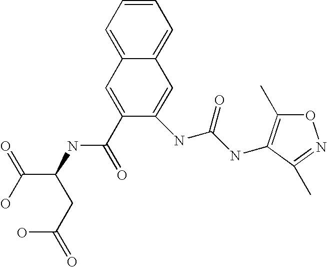 | N-{[3-({[(3,5-dimethyl-4- isoxazolyl) amino]carbonyl}amino)-2- naphthalenyl]carbonyl}-L-aspartic acid | 441.6 | 29.10 |
| 148 | 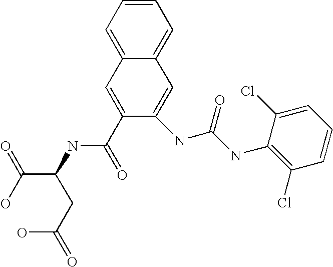 | N-{[3-({[(2,6- dichlorophenyl) amino]carbonyl}amino)-2- naphthalenyl]carbonyl}-L-aspartic acid | 490.4 | 0.23 |
| 149 | 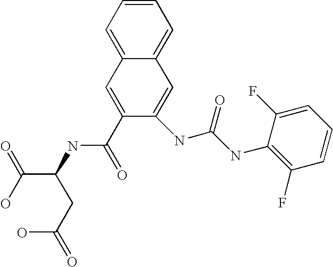 | N-{[3-({[(2,6- difluorophenyl) amino]carbonyl}amino)-2- naphthalenyl]carbonyl}-L-aspartic acid | 458.6 | 5.02 |
| 150 | 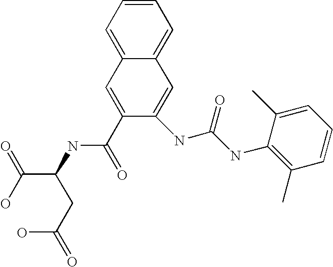 | N-{[3-({[(2,6- dimethylphenyl)amino]carbonyl}amino)-2- naphthalenyl]carbonyl}-L-aspartic acid | 450.4 | 0.12 |
| 151 | 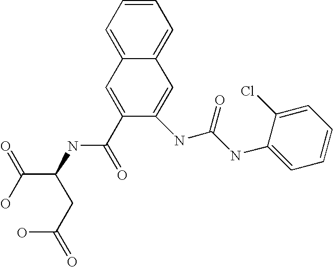 | N-{[3-({[(2-chlorophenyl) amino]carbonyl}amino)-2- naphthalenyl]carbonyl}-L-aspartic acid | 456.4 | 1.55 |
| 152 | 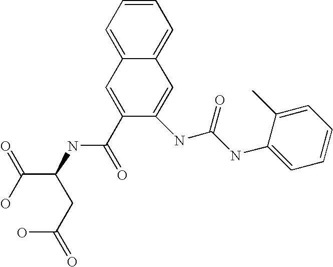 | N-{[3-({[(2-methylphenyl) amino]carbonyl}amino)-2- naphthalenyl]carbonyl}-L-aspartic acid | 436.4 | 1.23 |
| 153 | 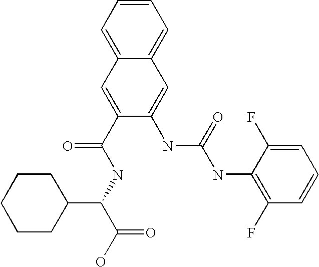 | (2S)-cyclohexyl({[3-({[(2,6- difluorophenyl) amino]carbonyl}amino)-2- naphthalenyl]carbonyl}amino)ethanoic acid | 482.2 | 4.85 |
| 154 | 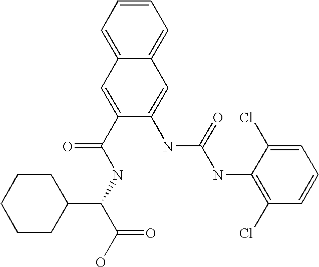 | (2S)-cyclohexyl({[3-({[(2,6- dichlorophenyl) amino]carbonyl}amino)-2- naphthalenyl]carbonyl}amino)acetic acid | 514.6 | 0.10 |
| 155 | 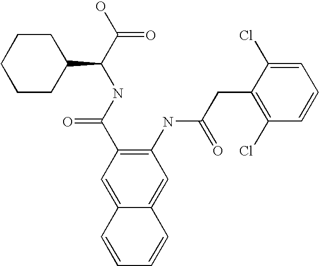 | (2S)-cyclohexyl{[(3-{[(2,6- dichlorophenyl) acetyl]amino}-2- naphthalenyl) carbonyl]amino}ethanoic acid | 513.6 | 6.80 |
| 156 | 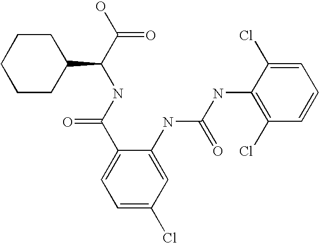 | (2S)({[4-chloro-2-({[(2,6- dichlorophenyl) amino]carbonyl}amino) phenyl]carbonyl}amino) (cyclohexyl) ethanoic acid | [M −H] =496 | 0.082 |
| 157 | 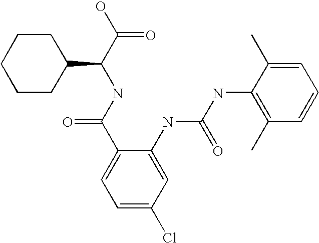 | (2S)-({[4-chloro-2-({[(2,6- dimethylphenyl)amino]carbonyl}amino)phenyl]carbonyl}amino)(cyclohexyl) ethanoic acid | [M −H] =456 | 0.242 |
| 158 | 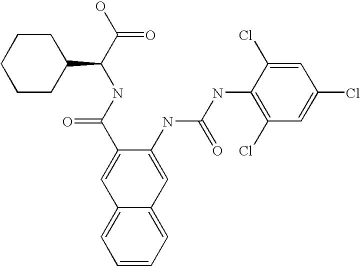 | (2S)-cyclohexyl{[3- ({[(2,4,6- trichlorophenyl)amino]carbonyl}amino)-2-naphthoyl]amino}ethanoic acid | [M −H] =546 | 0.079 |
| 159 | 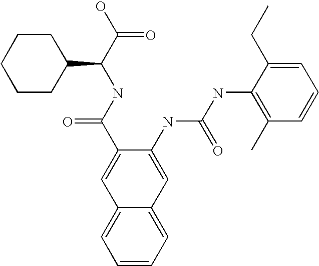 | (2S)-cyclohexyl{[3-({[(2- ethyl-6-methylphenyl) amino]carbonyl}amino)-2- naphthoyl]amino}ethanoic acid | [M −H] =486 | 0.058 |
| 160 | 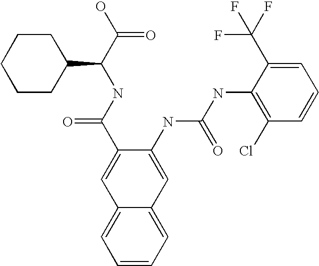 | (2S)-({3-[({[2-chloro-6- (trifluoromethyl)phenyl]amino}carbonyl)amino]-2- naphthoyl}amino) (cyclohexyl) ethanoic acid | [M −H] =546 | 0.068 |
| 161 | 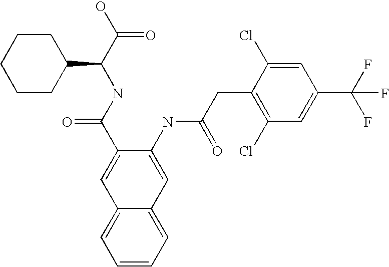 | (2S)-cyclohexyl{[3-({[2,6- dichloro-4- (trifluoromethyl)phenyl]acetyl}amino)-2-naphthoyl]amino}ethanoic acid | [M −H] =579 | 0.11 |
| 162 | 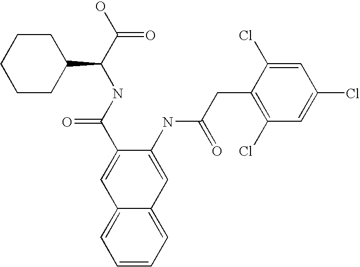 | (2S)-cyclohexyl[(3-{[(2,4,6- trichlorophenyl)acetyl]amino}-2-naphthoyl) amino]ethanoic acid | [M −H] =546 | 0.046 |
| 163 | 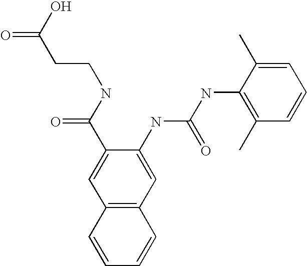 | N-[3-({[(2,6- dimethylphenyl)amino]carbonyl}amino)-2-naphthoyl]- beta-alanine | [M −H] =404 | 0.509 |
| 164 | 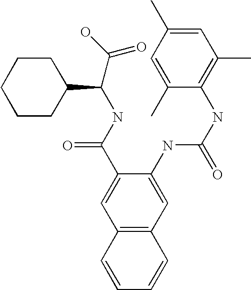 | (2S)-cyclohexyl[(3- {[(mesitylamino)carbonyl]amino}-2-naphthoyl) amino]ethanoic acid | [M −H] =486 | 0.006 |
| 165 | 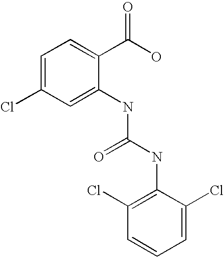 | 4-Chloro-2-({[(2,6- dichlorophenyl)amino]carbonyl}amino) benzoic acid | 358 (M −H) | 15.1 |
| 166 | 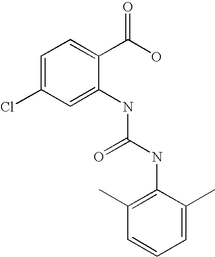 | 2-({[(2,6- dimethylphenyl)amino]carbonyl}amino)benzoic acid | 317 (M −H) | 8.64 |
| 167 | 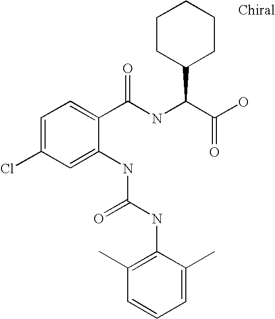 | (2S)-({[4-chloro-2-({[(2,6- dimethylphenyl)amino]carbonyl}amino)phenyl]carbonyl}amino)(cyclohexyl) ethanoic acid | 456 (M −H) | 0.39 |
| 168 | 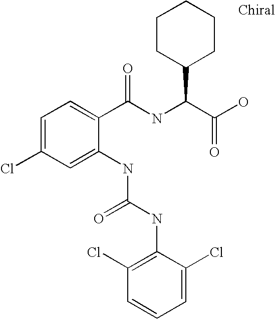 | (2S)-({[4-chloro-2-({[(2,6- dichlorophenyl)amino]carbonyl}amino)phenyl]carbonyl}amino)(cyclohexyl) ethanoic acid | 496 (M −H) | 0.2 |
| 169 | 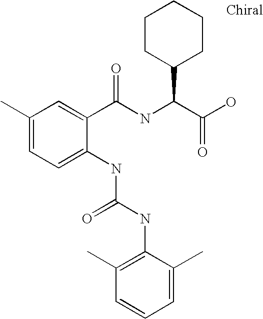 | (2S)Cyclohexyl({[2({[(2,6- methylphenyl)amino]carbonyl}amino)-5- methylphenyl]carbonyl}amino)ethanoic acid | 436 (M −H) | 0.68 |
| 170 | 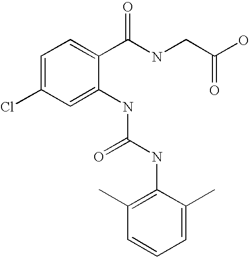 | N{[4chloro2({[(2,6 dimethylphenyl)amino]carbonyl}amino)phenyl]carbonyl}glycine | 374 (M −H) | 2.91 |
| 171 | 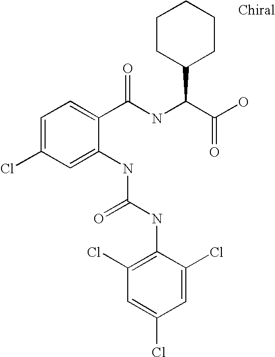 | (2S)-({[4-Chloro-2-({[(2,4,6- trichlorophenyl)amino]carbonyl}amino)phenyl]carbonyl}amino)(cyclohexyl) ethanoic acid | 532 (M −H) | 0.33 |
| 172 | 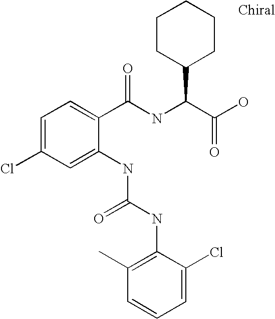 | (2S)-({[4-chloro-2-({[(2- chloro-6- methylphenyl)amino]carbonyl}amino)phenyl]carbonyl}amino)(cyclohexyl)ethanoic acid | 476 (M −H) | 0.18 |
| 173 | 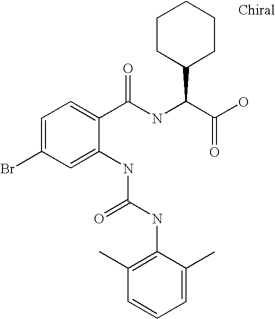 | (2S)-({[4-Bromo-2-({[(2,6- dimethylphenyl)amino]carbonyl}amino)phenyl]carbonyl}amino)(cyclohexyl) ethanoic acid | 502, 504 (M, M +2) | 0.31 |
| 174 | 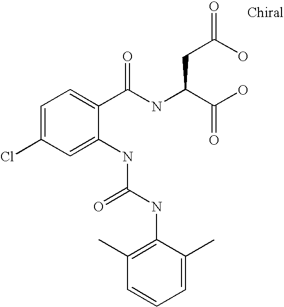 | N-{[4-Chloro-2-({[(2,6- dimethylphenyl)amino]carbonyl}amino)phenyl]carbonyl}-L-aspartic acid | 432 (M −H) | 0.77 |
| 175 | 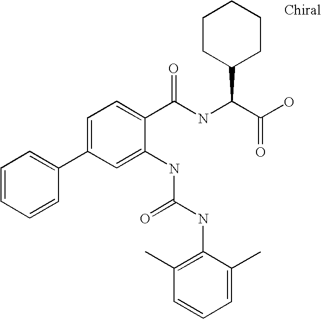 | (2S)-Cyclohexyl({[3-({[(2,6- dimethylphenyl)amino]carbonyl}amino)-4- biphenylyl]carbonyl}amino) ethanoic acid | 498 (M −H) | 0.163 |
| 176 | 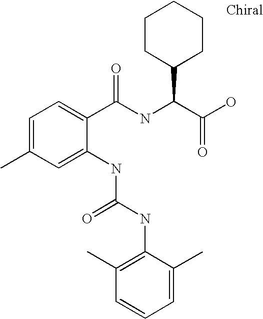 | (2S)-cyclohexyl({[2-({[(2,6- dimethylphenyl)amino]carbonyl}amino)-4- methylphenyl]carbonyl}amino)ethanoic acid | 436 (M −H) | 0.75 |
| 177 | 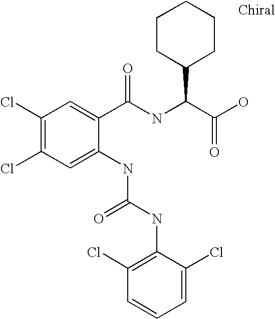 | (2S)-Cyclohexyl({[4,5- dichloro-2-({[(2,6- dichlorophenyl)amino]carbonyl}amino)phenyl]carbonyl}amino) ethanoic acid | 531 (M −H) | 0.1 |
| 178 | 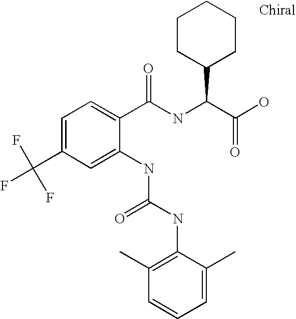 | (2S)-cyclohexyl({[2-({[(2,6- dimethylphenyl)amino]carbonyl}amino)-4- (trifluoromethyl)phenyl]carbonyl}amino)ethanoic acid | 490 (M −H) | 2.33 |
| 179 | 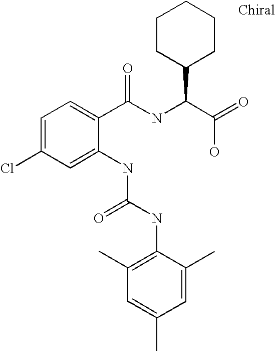 | (2S)-({[4-Chloro-2-({[(2,4,6- trimethylphenyl)amino]carbonyl}amino)phenyl]carbonyl}amino)(cyclohexyl)ethanoic acid | 470 (M −H) | 0.035 |
| 180 | 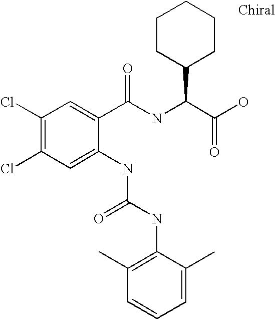 | (2S)-Cyclohexyl({[4,5- dichloro-2-({[(2,6- dimethylphenyl)amino]carbonyl}amino)phenyl]carbonyl}amino)ethanoic acid | 490 (M −H) | 0.094 |
| 181 | 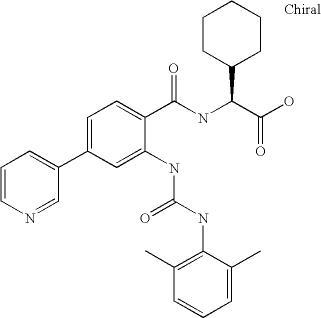 | (2S)-Cyclohexyl({[2-({[(2,6- dimethylphenyl)amino]carbonyl}amino)-4-(3- pyridinyl)phenyl]carbonyl}amino)ethanoic acid | 499 (M −H) | 0.099 |
| 182 | 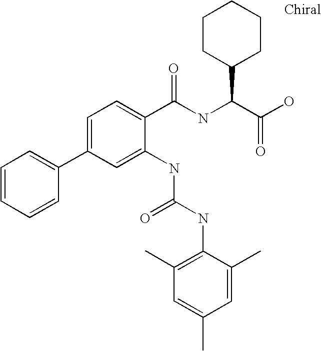 | (2S)-Cyclohexyl({[3- ({[(2,4,6- trimethylphenyl)amino]carbonyl}amino)-4- biphenylyl]carbonyl}amino) ethanoic acid | 512 (M −H) | 0.017 |
| 183 | 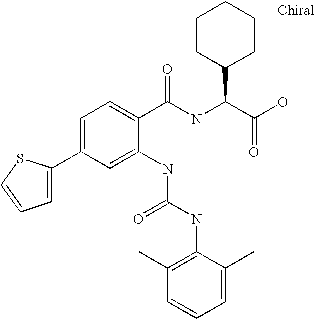 | (2S)-Cyclohexyl({[2-({[(2,6- dimethylphenyl)amino]carbonyl}amino)-4-(2- thienyl)phenyl]carbonyl}amino)ethanoic acid | 504 (M −H) | 0.1 |
| 184 | 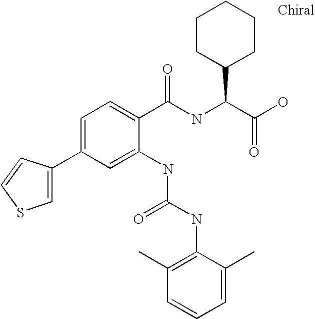 | (2S)-Cyclohexyl({[2-({[(2,6- dimethylphenyl)amino]carbonyl}amino)-4-(3- thienyl)phenyl]carbonyl}amino)ethanoic acid | 504 (M −H) | 0.22 |
| 185 | 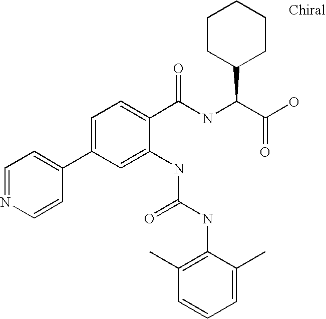 | (2S)-Cyclohexyl({[2-({[(2,6- dimethylphenyl)amino]carbonyl}amino)-4-(4- pyridinyl)phenyl]carbonyl}amino)ethanoic acid | 501 (M +H) | 0.17 |
| 186 | 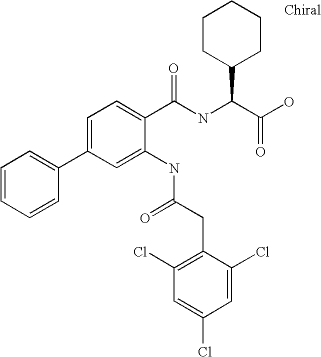 | (2S)-Cyclohexyl{[3- {[(2,4,6- trichlorophenyl)acetyl]amino}-4- biphenylyl)carbonyl]amino}ethanoic acid | 573 (M) | 0.341 |
| 187 | 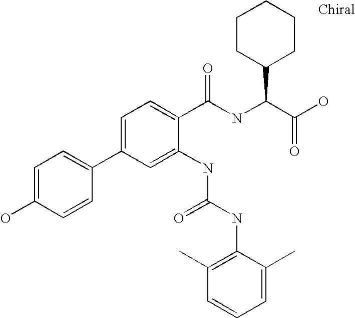 | (2S)-Cyclohexyl({[3-({[(2,6- dimethylphenyl)amino]carbonyl}amino)-4′-hydroxy-4- biphenylyl]carbonyl}amino) ethanoic acid | 516 (M +H) | 0.034 |
| 188 | 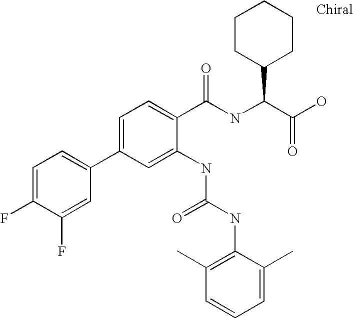 | (2S)-Cyclohexyl({[3-({[(2,6- dimethylphenyl)amino]carbonyl}amino)-3′,4′-difluoro- 4- biphenylyl]carbonyl}amino) ethanoic acid | 534 (M −H) | 0.074 |
| 189 | 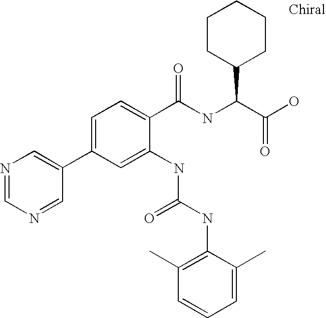 | (2S)-Cyclohexyl({[2-({[(2,6- dimethylphenyl)amino]carbonyl}amino)-4-(5- pyrimidinyl)phenyl]carbonyl}amino)ethanoic acid | 500 (M −H) | 0.33 |
| 190 | 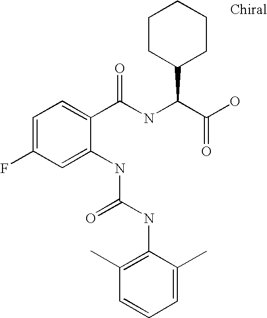 | (2S)-Cyclohexyl({[2-({[(2,6- dimethylphenyl)amino]carbonyl}amino)-4- fluorophenyl]carbonyl}amino) ethanoic acid | 440 (M −H) | 0.7 |
| 191 | 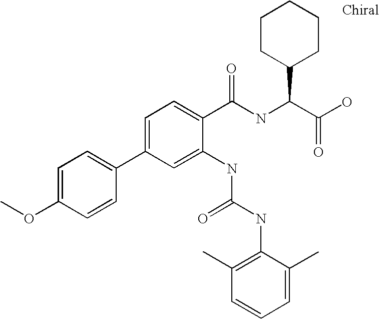 | (2S)-Cyclohexyl({[3-({[(2,6- dimethylphenyl)amino]carbonyl}amino)-4′- (methyloxy)-4- biphenylyl]carbonyl}amino) ethanoic acid | 530 (M +H) | 0.032 |
| 192 | 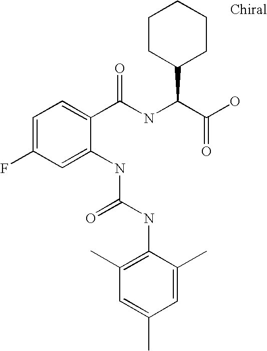 | (2S)-Cyclohexyl({[4-fluoro- 2-({[(2,4,6- trimethylphenyl)amino]carbonyl}amino)phenyl]carbonyl}amino)ethanoic acid | 456 (M +H) | 0.12 |
| 193 | 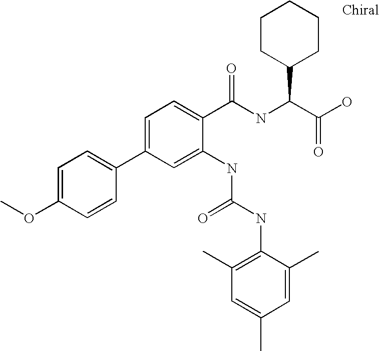 | (2S)-Cyclohexyl({[4′- (methyloxy)-3-({[(2,4,6- trimethylphenyl)amino]carbonyl}amino)-4- biphenylyl]carbonyl}amino) ethanoic acid | 542 (M −H) | 0.007 |
| 194 | 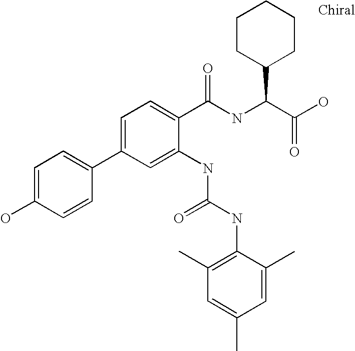 | 2S)-Cyclohexyl({[4′- hydroxy-3-({[(2,4,6- trimethylphenyl)amino]carbonyl}amino)-4- biphenylyl]carbonyl}amino) ethanoic acid | 530 (M +H) | 0.011 |
| 195 | 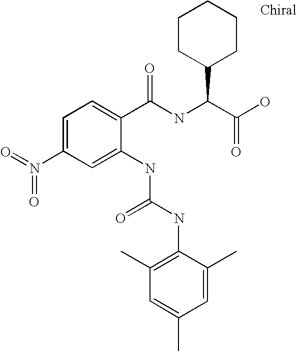 | (2S)-Cyclohexyl({[4-nitro-2- ({[(2,4,6- trimethylphenyl)amino]carbonyl}amino)phenyl]carbonyl}amino)ethanoic acid | 483 (M +H) | 0.55 |
| 196 | 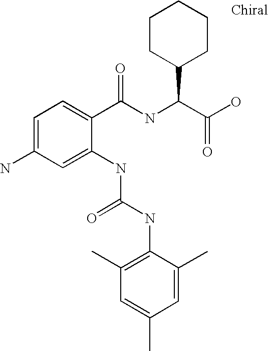 | (2S)-({[4-Amino-2-({[(2,4,6- trimethylphenyl)amino]carbonyl}amino)phenyl]carbonyl}amino)(cyclohexyl) ethanoic acid | 453 (M +H) | 0.11 |
| 197 | 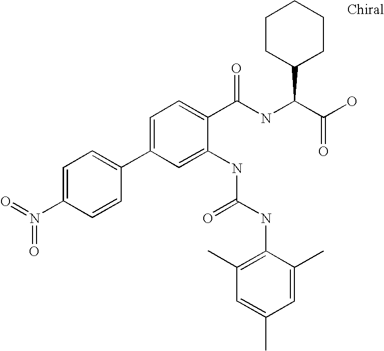 | (2S)-Cyclohexyl({[4′-nitro- 3-({[(2,4,6- trimethylphenyl)amino]carbonyl}amino)-4- biphenylyl]carbonyl}amino) ethanoic acid | 559 (M +H) | 0.014 |
| 198 | 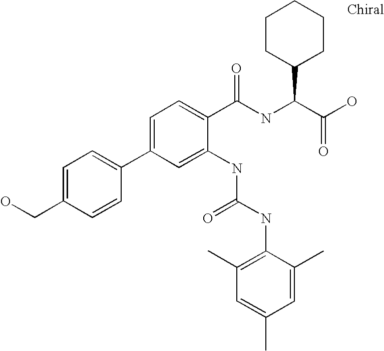 | (2S)-Cyclohexyl({[4′- (hydroxymethyl)-3- ({[(2,4,6- trimethylphenyl)amino]carbonyl}amino)-4- biphenylyl]carbonyl}amino) ethanoic acid | 542 (M −H) | 0.027 |
| 199 | 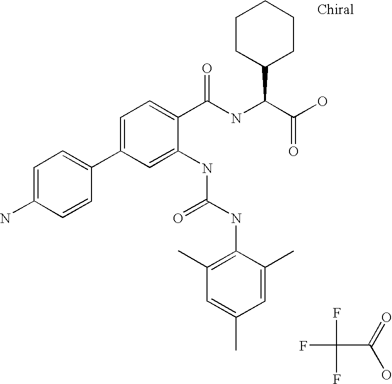 | (2S)-({[4′-Amino-3- ({[(2,4,6- trimethylphenyl)amino]carbonyl}amino)-4- biphenylyl]carbonyl}amino) (cyclohexyl)ethanoic acid | 529 (M +H) | 0.004 |
| 200 | 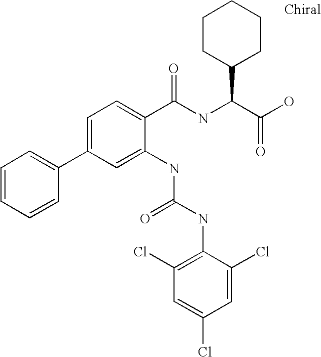 | (2S)-Cyclohexyl({[3- ({[(2,4,6- trichlorophenyl)amino]carbonyl}amino)-4- biphenylyl]carbonyl}amino) ethanoic acid | 574 (M) | 0.13 |
| 201 | 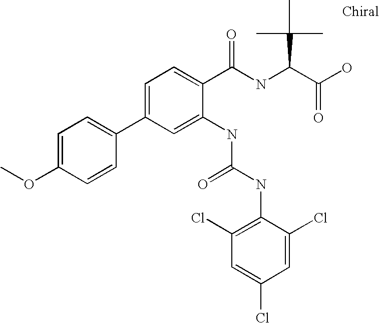 | 3-Methyl-N-{[4′- (methyloxy)-3-({[(2,4,6- trichlorophenyl)amino]carbonyl}amino)-4- biphenylyl]carbonyl}-L- valine | 577 (M −H) | 0.67 |
| 202 | 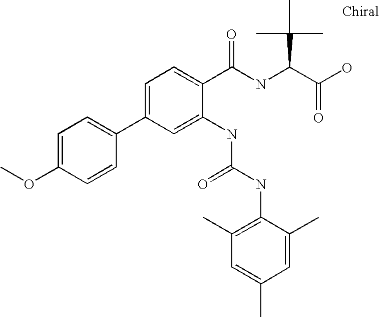 | 3-Methyl-N-{[4′- (methyloxy)-3-({[(2,4,6- trimethylphenyl)amino]carbonyl}amino)-4- biphenylyl]carbonyl}-L- valine | 518 (M +H) | 0.14 |
| 203 | 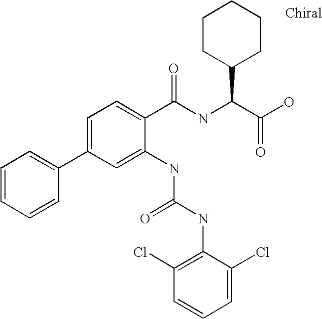 | (2S)-Cyclohexyl({[3-({[(2,6- dichlorophenyl)amino]carbonyl}amino)-4- biphenylyl]carbonyl}amino) ethanoic acid | 538 (M −H) | 0.075 |
| 204 | 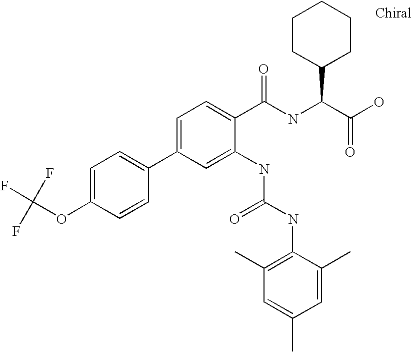 | (2S)-Cyclohexyl({[4′- [(trifluoromethyl)oxy]-3- ({[(2,4,6- trimethylphenyl)amino]carbonyl}amino)-4- biphenylyl]carbonyl}amino) ethanoic acid | 598 (M +H) | 0.19 |
| 205 | 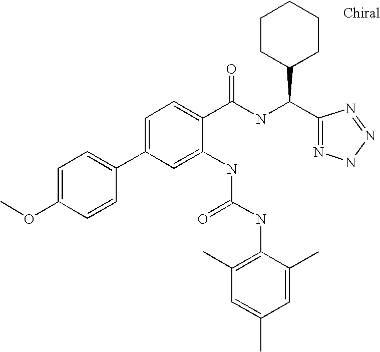 | N-[(S)-cyclohexyl(1H- tetrazol-5-yl)methyl]-4′- (methyloxy)-3-({[(2,4,6- trimethylphenyl)amino]carbonyl}amino)-4- biphenylcarboxamide | 566 (M −H) | 0.14 |
| 206 | 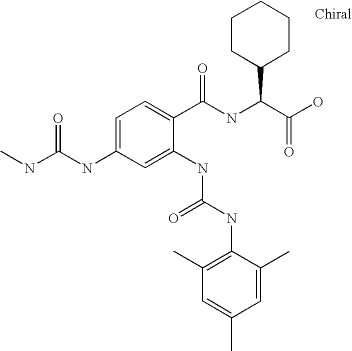 | (2S)-Cyclohexyl({[4- {[(methylamino)carbonyl]amino}-2-({[(2,4,6- trimethylphenyl)amino]carbonyl}amino)phenyl]carbonyl}amino)ethanoic acid | 510 (M +H) | 0.096 |
| 207 | 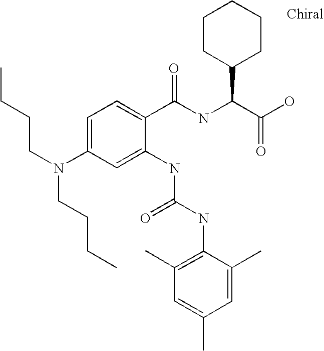 | (2S)-Cyclohexyl({[4- (dibutylamino)-2-({[(2,4,6- carbonyl}amino)phenyl]carbonyl}amino)ethanoic acid | 565 (M +H) | 16.1 |
| 208 | 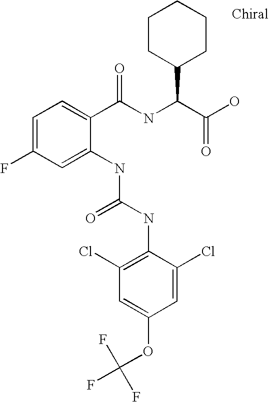 | (2S)-Cyclohexyl{[(2-{[({2,6- dichloro-4- [(trifluoromethyl)oxy]phenyl}amino)carbonyl]amino}-4- fluorophenyl)carbonyl]amino}ethanoic acid | 564 (M −H) | 0.13 |
| 209 | 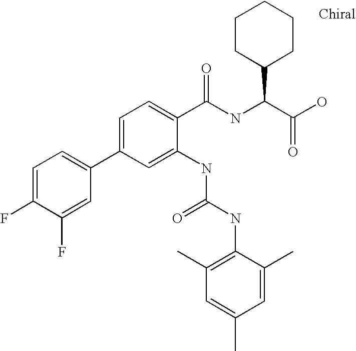 | (2S)-Cyclohexyl({[3′,4′- difluoro-3-({[(2,4,6- trimethylphenyl)amino]carbonyl}amino)-4- biphenylyl]carbonyl}amino) ethanoic acid | 550 (M +H) | 0.014 |
| 210 | 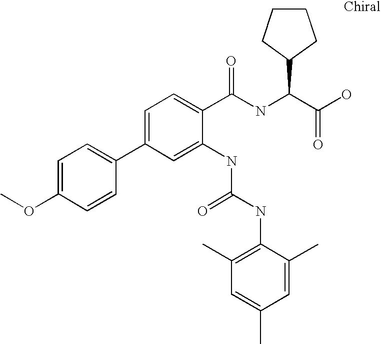 | (2S)-Cyclopentyl({[4′- (methyloxy)-3-({[(2,4,6- trimethylphenyl)amino]carbonyl}amino)-4- biphenylyl]carbonyl}amino) ethanoic acid | 528 (M −H) | 0.014 |
| 211 | 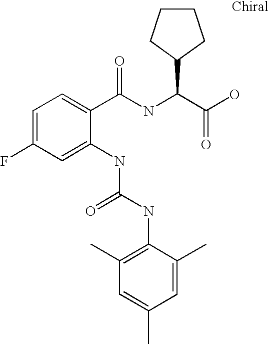 | (2S)-Cyclopentyl({[4-fluoro- 2-({[(2,4,6- trimethylphenyl)amino]carbonyl}amino)phenyl]carbonyl}amino)ethanoic acid | 440 (M −H) | 0.29 |
| 212 | 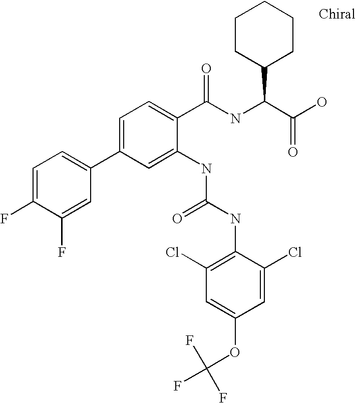 | (2S)-Cyclohexyl{[(3-{[({2,6- dichloro-4- [(trifluoromethyl)oxy]phenyl}amino)carbonyl]amino}- 3′,4′-difluoro-4- biphenylyl)carbonyl]amino}ethanoic acid | 658 (M −H) | 0.017 |
| 213 | 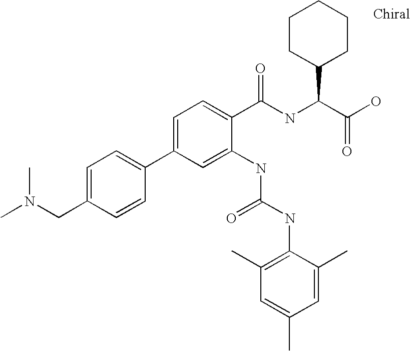 | (2S)-Cyclohexyl({[4′- [(dimethylamino)methyl]-3- ({[(2,4,6- trimethylphenyl)amino]carbonyl}amino)-4- biphenylyl]carbonyl}amino) ethanoic acid | 571 (M +H) | 0.024 |
| 214 | 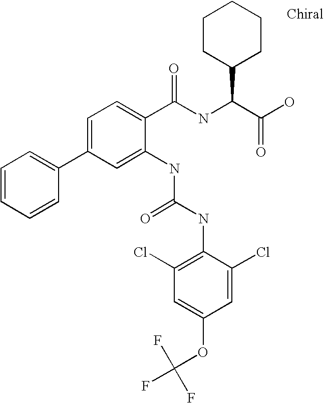 | (2S)-Cyclohexyl{[(3-{[({2,6- dichloro-4- [(trifluoromethyl)oxy]phenyl}amino)carbonyl]amino}-4- biphenylyl)carbonyl]amino}ethanoic acid | 622 (M −H) | 0.023 |
| 215 | 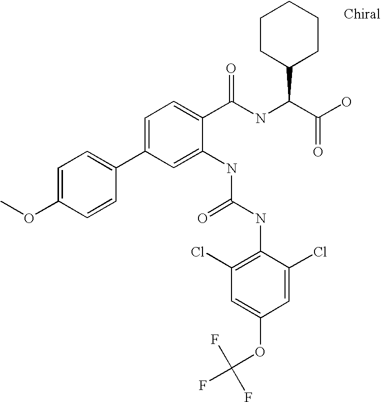 | (2S)-Cyclohexyl({[3-{[({2,6- dichloro-4- [(trifluoromethyl)oxy]phenyl}amino)carbonyl]amino}-4′- (methyloxy)-4- biphenylyl]carbonyl}amino) ethanoic acid | 652 (M −H) | 0.009 |
| 216 | 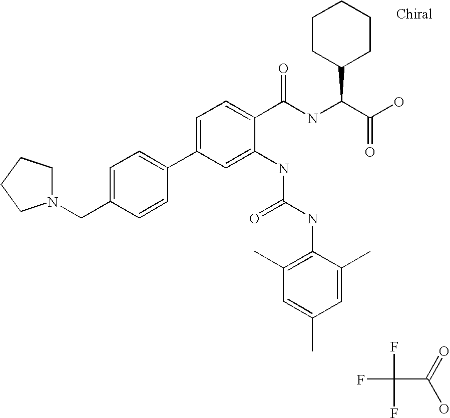 | (2S)-Cyclohexyl({[4′-(1- pyrrolidinylmethyl)-3- ({[(2,4,6- trimethylphenyl)amino]carbonyl}amino)-4- biphenylyl]carbonyl}amino) ethanoic acid | 595 (M −H) | 0.006 |
| 217 | 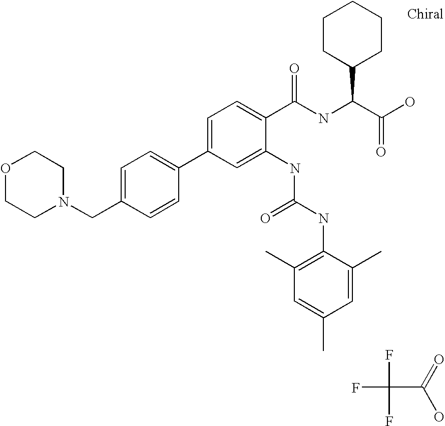 | (2S)-cyclohexyl({[4′-(4- morpholinylmethyl)-3- ({[(2,4,6- trimethylphenyl)amino]carbonyl}amino)-4- biphenylyl]carbonyl}amino) ethanoic acid | 611 (M −H) | 0.009 |
| 218 | 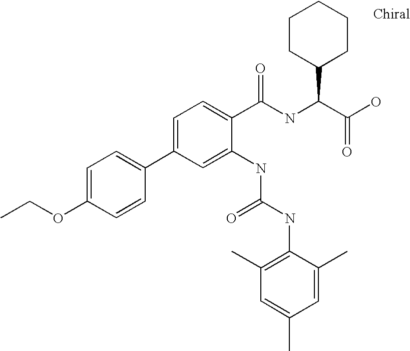 | (2S)-Cyclohexyl({[4′- (ethyloxy)-3-({[(2,4,6- trimethylphenyl)amino]carbonyl}amino)-4- biphenylyl]carbonyl}amino) ethanoic acid | 556 (M −H) | 0.007 |
| 219 | 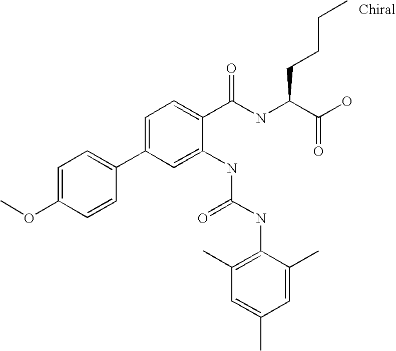 | N-{[4′-(methyloxy)-3- ({[(2,4,6- trimethylphenyl)amino]carbonyl}amino)-4- biphenylyl]carbonyl}-L- norleucine | 516 (M −H) | 0.015 |
| 220 | 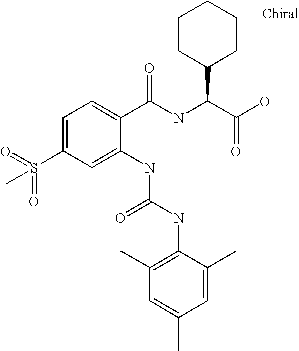 | (2S)-Cyclohexyl({[4- (methylsulfonyl)-2-({[(2,4,6- trimethylphenyl)amino]carbonyl}amino)phenyl]carbonyl}amino)ethanoic acid | 514 (M −H) | 1.96 |
| 221 | 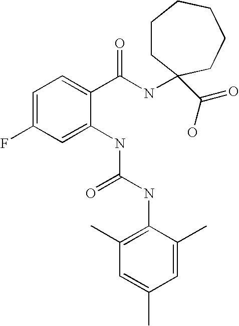 | -({[4-Fluoro-2-({[(2,4,6- trimethylphenyl)amino]carbonyl}amino)phenyl]carbonyl}amino)cycloheptanecarboxylic acid | 454 (M −H) | 0.3 |
| 222 | 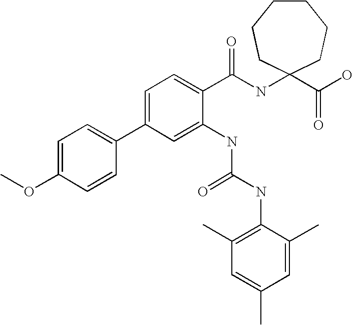 | 1-({[4′-(methyloxy)-3- ({[(2,4,6- trimethylphenyl)amino]carbonyl}amino)-4- biphenylyl]carbonyl}amino) cycloheptanecarboxylic acid | 542 (M −H) | 0.024 |
| 223 | 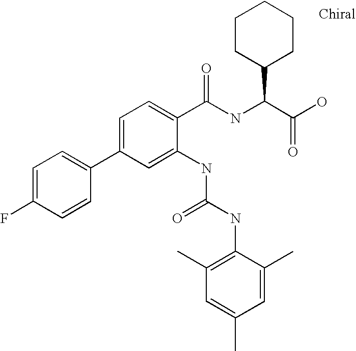 | (2S)-Cyclohexyl({[4′-fluoro- 3-({[(2,4,6- trimethylphenyl)amino]carbonyl}amino)-4- biphenylyl]carbonyl}amino) ethanoic acid | 530 (M −H) | 0.009 |
| 224 | 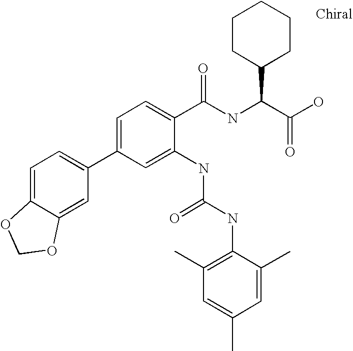 | (2S)-({[4-(1,3-Benzodioxol- 5-yl)-2-({[(2,4,6- trimethylphenyl)amino]carbonyl}amino)phenyl]carbonyl}amino)(cyclohexyl)ethanoic acid | 556 (M −H) | 0.003 |
| 225 | 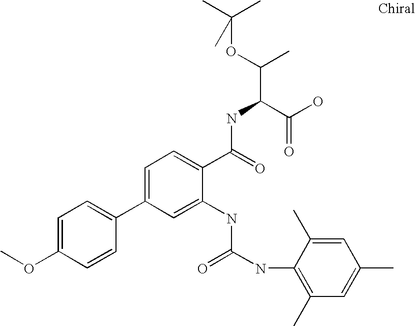 | O-(1,1-Dimethylethyl)-N- {[4′-(methyloxy)-3-({[(2,4,6- trimethylphenyl)amino]carbonyl}amino)-4- biphenylyl]carbonyl}-L- threonine | 560 (M −H) | 0.002 |
| 226 | 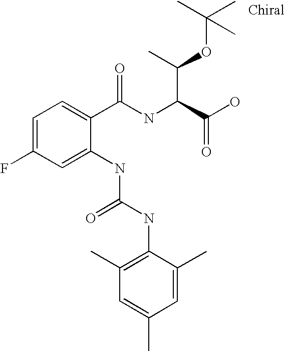 | O-(1,1-Dimethylethyl)-N- {[4-fluoro-2-({[(2,4,6- trimethylphenyl)amino]carbonyl}amino)phenyl]carbonyl}-L-threonine | 472 (M −H) (APCI) | 0.53 |
| 227 | 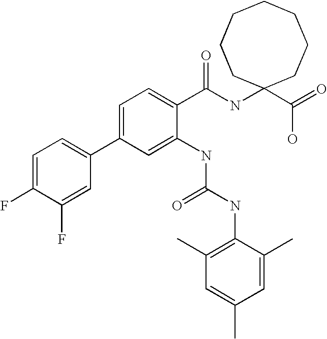 | 1-({[3′,4′-Difluoro-3- ({[(2,4,6- trimethylphenyl)amino]carbonyl}amino)-4- biphenylyl]carbonyl}amino) cyclooctanecarboxylic acid | 564 (M +H) | 0.001 |
| 228 | 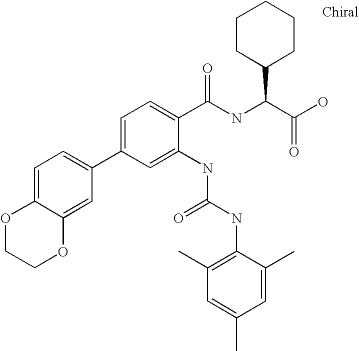 | (2S)-Cyclohexyl({[4-(2,3- dihydro-1,4-benzodioxin-6- yl)-2-({[2,4,6- trimethylphenyl)amino]carbonyl}amino)phenyl]carbonyl}amino)ethanoic acid | 572 (M +H) | 0.004 |
| 229 | 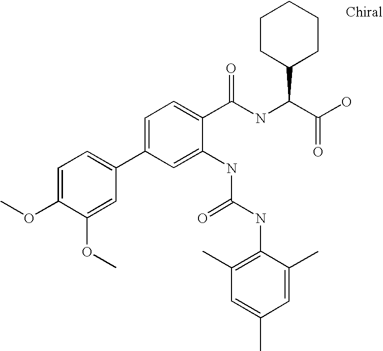 | (2S)-({[3′,4′- Bis(methyloxy)-3-({[(2,4,6- trimethylphenyl)amino]carbonyl}amino)-4- biphenylyl]carbonyl}amino) (cyclohexyl)ethanoic acid | 574 (M +H) | 0.003 |
| 230 | 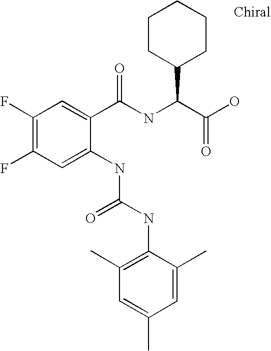 | (2S)-Cyclohexyl({[4,5- difluoro-2-({[(2,4,6- trimethylphenyl)amino]difluoro-2-({[(2,4,6- carbonyl}amino)phenyl]carbonyl}amino)ethanoic acid | 474 (M +H) | 0.058 |
| 231 | 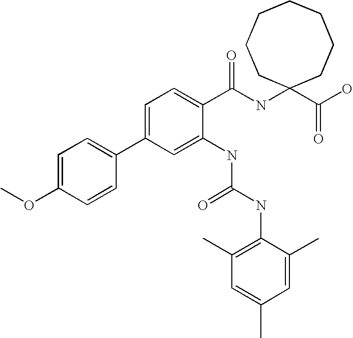 | 1-({[4′-(Methyloxy)-3- ({[(2,4,6- trimethylphenyl)amino]carbonyl}amino)-4- biphenylyl]carbonyl}amino) cyclooctanecarboxylic acid | 558 (M +H) | 0.001 |
| 232 | 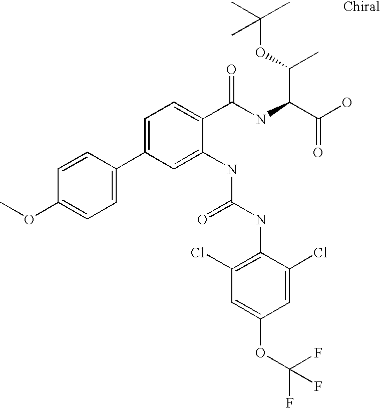 | N-{[3-{[({2,6-Dichloro-4- [(trifluoromethyl)oxy]phenyl}amino)carbonyl]amino}-4′- (methyloxy)-4- biphenylyl]carbonyl}-O- (1,1-dimethylethyl)-L- threonine | 672 (M +H) | 0.007 |
| 233 | 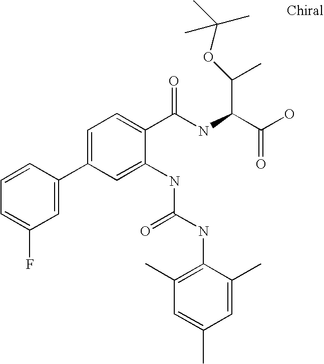 | O-(1,1-Dimethylethyl)-N- {[3′-fluoro-3-({[(2,4,6- trimethylphenyl)amino]carbonyl}amino)-4- biphenylyl]carbonyl}-L- threonine | 550 (M +H) | 0.023 |
| 234 | 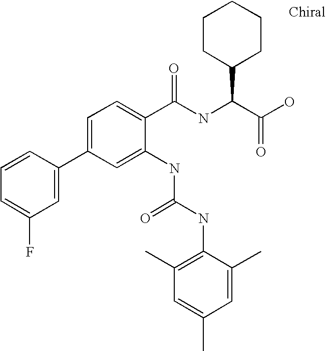 | (2S)-Cyclohexyl({[3′-fluoro- 3-({[(2,4,6- trimethylphenyl)amino]carbonyl}amino)-4- biphenylyl]carbonyl}amino) ethanoic acid | 532 (M +H) | 0.024 |
| 235 | 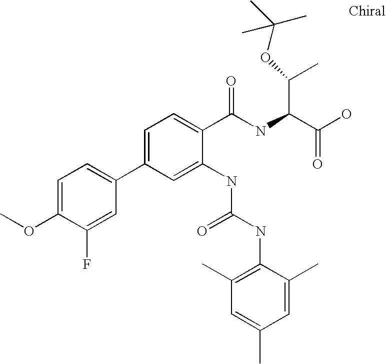 | O-(1,1-Dimethylethyl)-N- {[3′-fluoro-4′-(methyloxy)-3- ({[(2,4,6- trimethylphenyl)amino]carbonyl}amino)-4- biphenylyl]carbonyl}-L- threonine | 580 (M +H) | 0.013 |
| 236 | 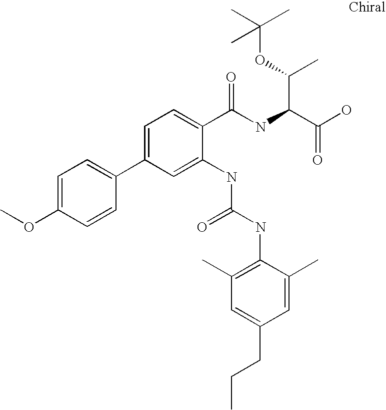 | O-(1,1-Dimethylethyl)-N- {[3-({[(2,6-dimethyl-4- propylphenyl)amino]carbonyl}amino)-4′-(methyloxy)-4- biphenylyl]carbonyl}-L- threonine | 590 (M +H) | 0.004 |
| 237 | 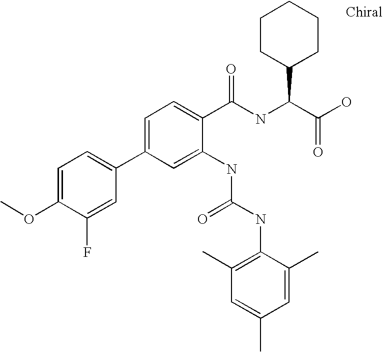 | (2S)-Cyclohexyl({[3′-fluoro- 4′-(methyloxy)-3-({[(2,4,6- trimethylphenyl)amino]carbonyl}amino)-4- biphenylyl]carbonyl}amino) ethanoic acid | 562 (M +H) (APCI) | 0.02 |
| 238 | 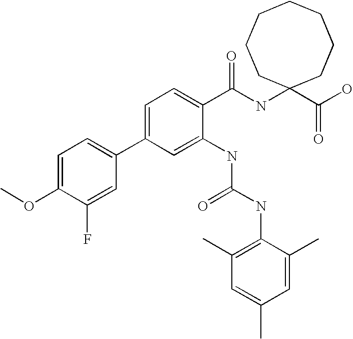 | 1-({[3′-Fluoro-4′- (methyloxy)-3-({[(2,4,6- trimethylphenyl)amino]carbonyl}amino)-4- biphenylyl]carbonyl}amino) cyclooctanecarboxylic acid | 574 (M −H) | 0.01 |
| 239 | 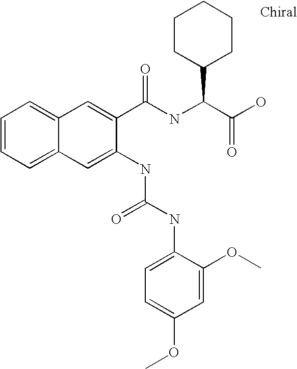 | (2S)-[({3-[({[2,4- (1 bis(methyloxy)phenyl]amino}carbonyl)amino]-2- naphthalenyl}carbonyl) amino](cyclohexyl)ethanoic acid | 506 (M +H) | 2.38 |
| 240 | 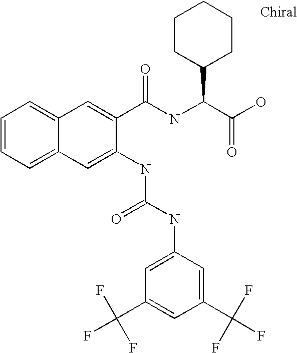 | (2S)-[({3-[({[3,5- bis(trifluoromethyl)phenyl]amino}carbonyl)amino]-2- naphthalenyl}carbonyl) amino](cyclohexyl)ethanoic acid | 582 (M +H) | 8.18 |
| 241 | 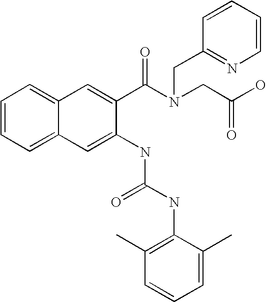 | N-{[3-({[(2,6- dimethylphenyl)amino]carbonyl}amino)-2- naphthalenyl]carbonyl}-N- (2-pyridinylmethyl)glycine | 483 (M +H) | 3.61 |
| 242 | 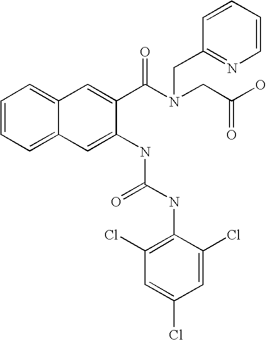 | N-(2-pyridinylmethyl)-N-{[3- ({[(2,4,6- trichlorophenyl)amino]carbonyl}amino)-2- naphthalenyl]carbonyl}glycine | 557 (M +H) | 2.86 |
| 243 | 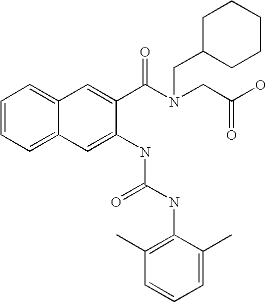 | N-(cyclohexylmethyl)-N- {[3-({[(2,6- dimethylphenyl)amino]carbonyl}amino)-2- naphthalenyl]carbonyl}glycine | 488 (M +H) | 1.77 |
| 244 | 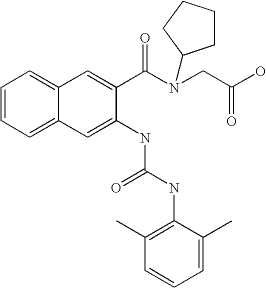 | N-cyclopentyl-N-{[3-({[(2,6- dimethylphenyl)amino]carbonyl}amino)-2- naphthalenyl]carbonyl}glycine | 460 (M +H) | 0.97 |
| 245 | 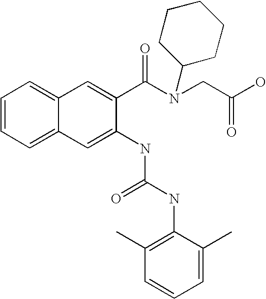 | N-cyclohexyl-N-{[3-({[(2,6- dimethylphenyl)amino]carbonyl}amino)-2- naphthalenyl]carbonyl}glycine | 474 (M +H) | 2.13 |
| 246 | 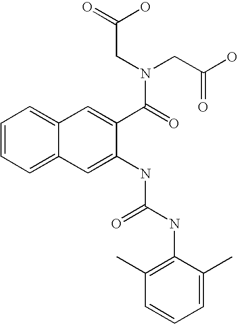 | 2,2′-({[3-({[(2,6- dimethylphenyl)amino]carbonyl}amino)-2- naphthalenyl]carbonyl}imino)diacetic acid (non- preferred name) | 450 (M +H) | 2.12 |
| 247 | 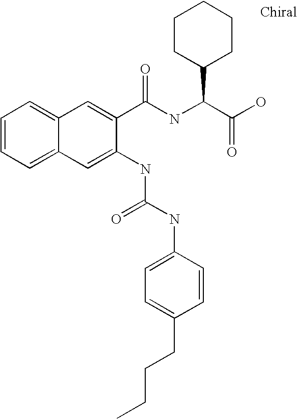 | (2S)-({[3-({[(4- butylphenyl)amino]carbonyl}amino)-2- naphthalenyl]carbonyl}amino)(cyclohexyl)ethanoic acid | 502 (M +H) | 10.42 |
| 248 | 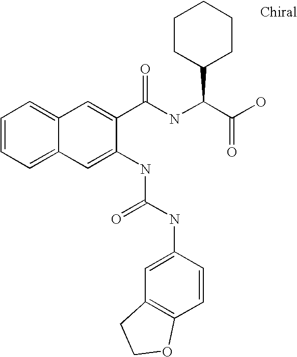 | (2S)-cyclohexyl{[(3-{[(2,3- dihydro-1-benzofuran-5- ylamino)carbonyl]amino}-2- naphthalenyl)carbonyl]amino}ethanoic acid | 488 (M +H) | 15.13 |
| 249 | 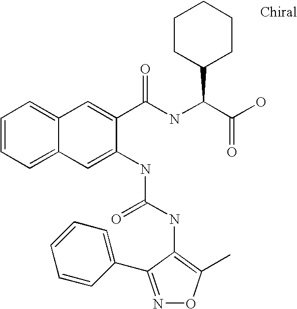 | (2S)-cyclohexyl({[3-({[(5- methyl-3-phenyl-4- isoxazolyl)amino]carbonyl}amino)-2- naphthalenyl]carbonyl}amino)ethanoic acid | 527 (M +H) | 17.4 |
| 250 | 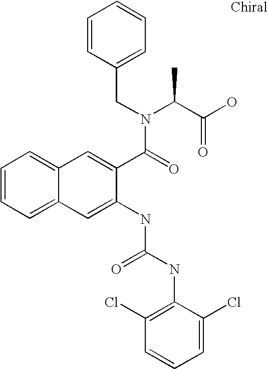 | N-{[3-({[(2,6- dichlorophenyl)amino]carbonyl}amino)-2- naphthalenyl]carbonyl}-N- (phenylmethyl)-L-alanine | 536 (M +H) | 7.77 |
| 251 | 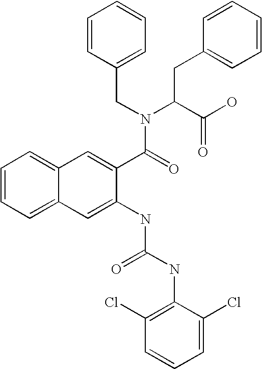 | N-{[3-({[(2,6- dichlorophenyl)amino]carbonyl}amino)-2- naphthalenyl]carbonyl}-N- (phenylmethyl)phenylalanine | 611 (M −H) | 12.2 |
| 252 | 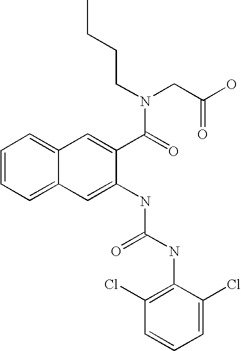 | N-butyl-N-{[3-({[(2,6- dichlorophenyl)amino]carbonyl}amino)-2- naphthalenyl]carbonyl}glycine | 488 (M +H) | 0.65 |
| 253 | 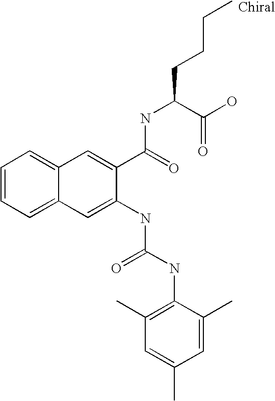 | N-{[3-({[(2,4,6- trimethylphenyl)amino]carbonyl}amino)-2- naphthalenyl]carbonyl}-L- norleucine | 462 (M +H) | 0.016 |
| 254 | 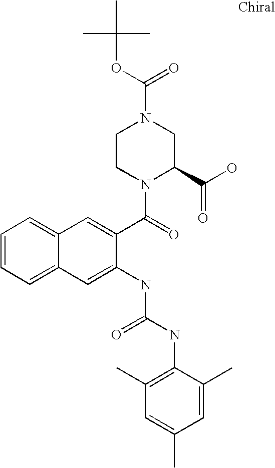 | (2S)-4-{[(1,1- dimethylethyl)oxy]carbonyl}- 1-{[3-({[(2,4,6- trimethylphenyl)amino]carbonyl}amino)-2- naphthalenyl]carbonyl}-2- piperazinecarboxylic acid | 561 (M +H) | 0.81 |
| 255 | 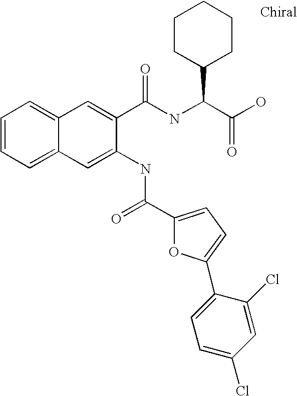 | (2S)-cyclohexyl({[3-({[5- (2,4-dichlorophenyl)-2- furanyl]carbonyl}amino)-2- naphthalenyl]carbonyl}amino)ethanoic acid | 563 (M −H) | 2.6 |
| 256 | 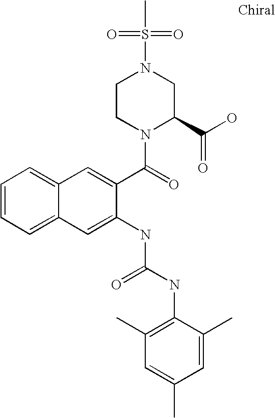 | (2S)-4-(methylsulfonyl)-1- {[3-({[(2,4,6- trimethylphenyl)amino]carbonyl}amino)-2- naphthalenyl]carbonyl}-2- piperazinecarboxylic acid | 539 (M +H) | 17.42 |
| 257 | 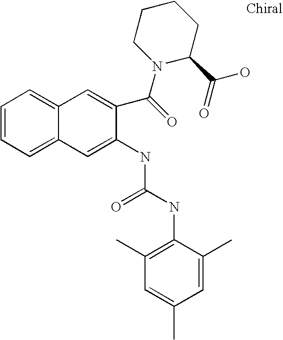 | (2S)-1-{[3-({[(2,4,6- trimethylphenyl)amino]carbonyl}amino)-2- naphthalenyl]carbonyl}-2- piperidinecarboxylic acid | 460 (M +H) | 18.43 |
| 258 | 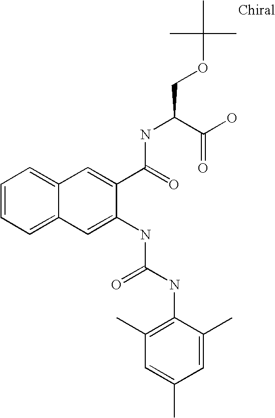 | O-(1,1-dimethylethyl)-N- {[3-({[(2,4,6- trimethylphenyl)amino]carbonyl}amino)-2- naphthalenyl]carbonyl}-L- serine | 492 (M +H) | 0.018 |
| 259 | 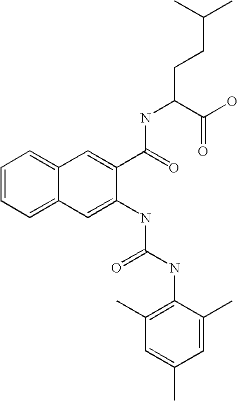 | 5-methyl-N-{[3-({[(2,4,6- trimethylphenyl)amino]carbonyl}amino)-2- naphthalenyl]carbonyl}norleucine | 476 (M +H) | 0.056 |
| 260 | 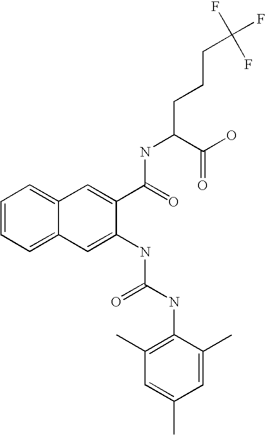 | 6,6,6-trifluoro-N-{[3- ({[(2,4,6- trimethylphenyl)amino]carbonyl}amino)-2- naphthalenyl]carbonyl}norleucine | 516 (M +H) | 0.067 |
| 261 | 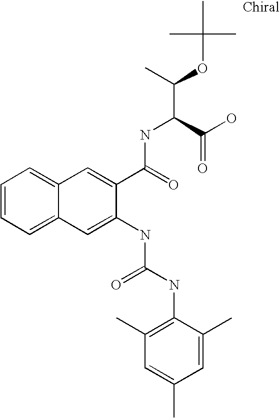 | O-(1,1-dimethylethyl)-N- {[3-({[(2,4,6- trimethylphenyl)amino]carbonyl}amino)-2- naphthalenyl]carbonyl}-L- threonine | 506 (M +H) | 0.005 |
| 262 | 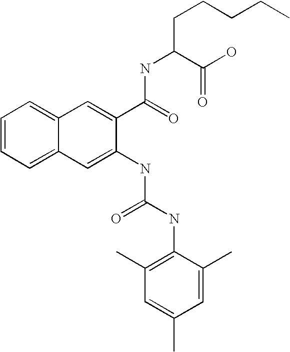 | 2-({[3-({[(2,4,6- trimethylphenyl)amino]carbony}amino)-2- naphthalenyl]carbonyl}amino)heptanoic acid | 476 (M +H) | 0.14 |
| 263 | 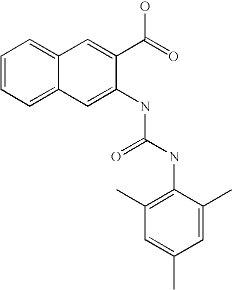 | 3-({[(2,4,6- trimethylphenyl)amino]carbonyl}amino)-2- naphthalenecarboxylic acid | 349 (M +H) | 3.46 |
| 264 | 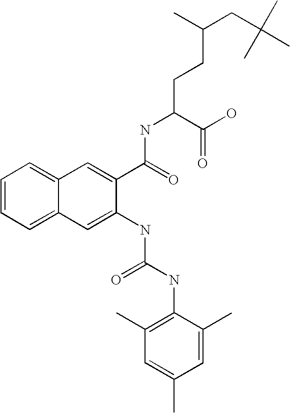 | 5,7,7-trimethyl-2-({[3- ({[(2,4,6- trimethylphenyl)amino]carbonyl}amino)-2- naphthalenyl]carbonyl}amino)octanoic acid | 532 (M +H) | 0.12 |
| 265 | 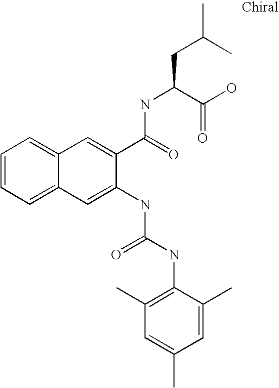 | N-{[3-({[(2,4,6- trimethylphenyl)amino]carbonyl}amino)-2- naphthalenyl]carbonyl}-L- leucine | 462 (M +H) | 0.07 |
| 266 | 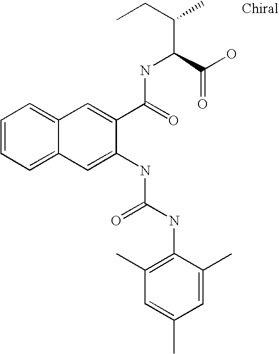 | N-{[3-({[(2,4,6- trimethylphenyl)amino]carbonyl}amino)-2- naphthalenyl]carbonyl}-L- isoleucine | 462 (M +H) | 0.052 |
| 267 | 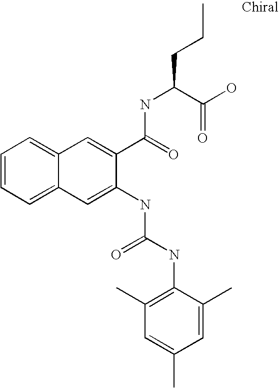 | N-{[3-({[(2,4,6- trimethylphenyl)amino]carbonyl}amino)-2- naphthalenyl]carbonyl}-L- norvaline | 448 (M +H) | 0.026 |
| 268 | 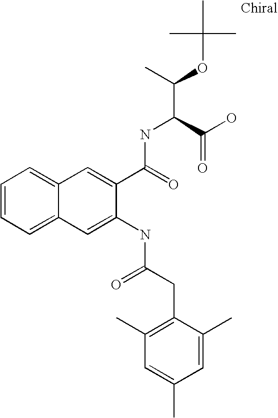 | O-(1,1-dimethylethyl)-N- [(3-{[(2,4,6- trimethylphenyl)acetyl]amino}-2- naphthalenyl)carbonyl]-L- threonine | 505 (M +H) | 0.05 |
| 269 | 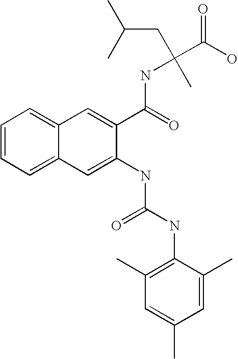 | 2-methyl-N-{[3-({[(2,4,6- trimethylphenyl)amino]carbonyl}amino)-2- naphthalenyl]carbonyl}leucine | 476 (M +H) | 0.21 |
| 270 | 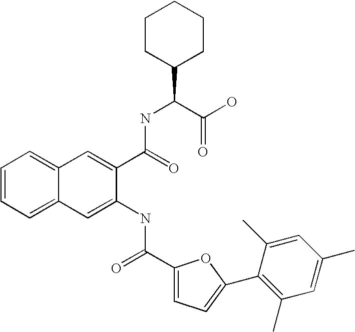 | (2S)-cyclohexyl({[3-({[5- (2,4,6-trimethylphenyl)-2- furanyl]carbonyl}amino)-2- naphthalenyl]carbonyl}amino)ethanoic acid | 539 (M +H) | 10.3 |
| 271 | 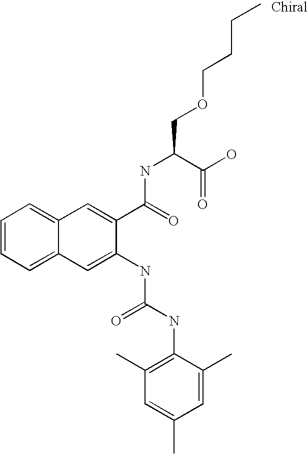 | O-butyl-N-{[3-({[(2,4,6- trimethylphenyl)amino]carbonyl}amino)-2- naphthalenyl]carbonyl}-L- serine | 492 (M +H) | 0.053 |
| 272 | 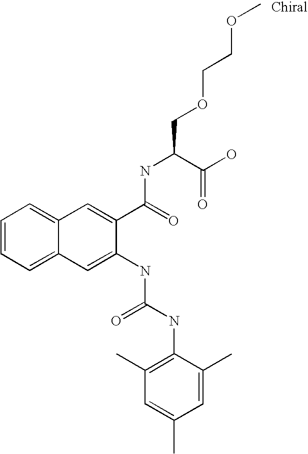 | O-[2-(methyloxy)ethyl]-N- {[3-({[(2,4,6- trimethylphenyl)amino]carbonyl}amino)-2- naphthalenyl]carbonyl}-L- serine | 494 (M +H) | 0.038 |
| 273 | 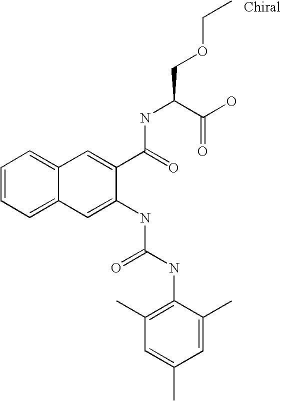 | O-ethyl-N-{[3-({[(2,4,6- trimethylphenyl)amino]carbonyl}amino)-2- naphthalenyl]carbonyl}-L- serine | 464 (M +H) | 0.056 |
| 274 | 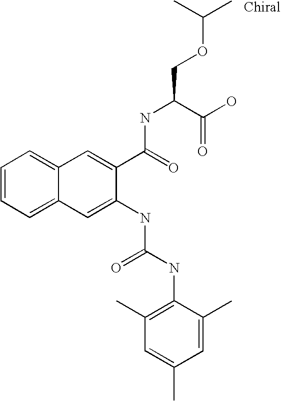 | O-(1-methylethyl)-N-{[3- ({[(2,4,6- trimethylphenyl)amino]carbonyl}amino)-2- naphthalenyl]carbonyl}-L- serine | 478 (M +H) | 0.04 |
| 275 | 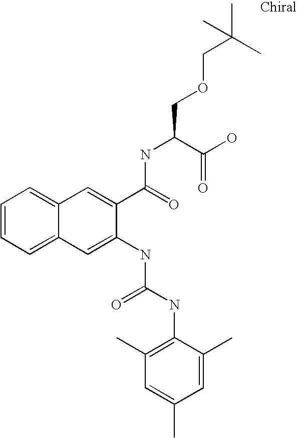 | O-(2,2-dimethylpropyl)-N- {[3-({[(2,4,6- trimethylphenyl)amino]carbonyl}amino)-2- naphthalenyl]carbonyl}-L- serine | 506 (M +H) | 0.041 |
| 276 | 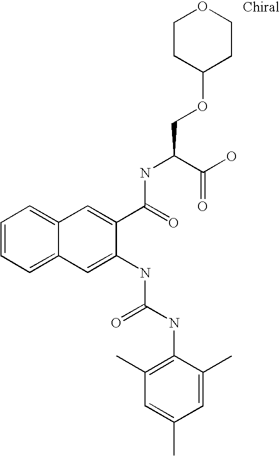 | O-(tetrahydro-2H-pyran-4- yl)-N-{[3-({[(2,4,6- trimethylphenyl)amino]carbonyl}amino)-2- naphthalenyl]carbonyl}-L- serine | 520 (M +H) | 0.045 |
| 277 | 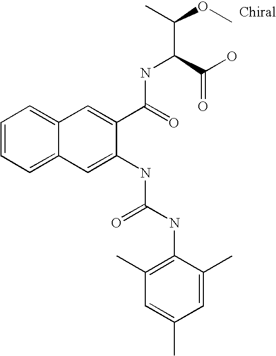 | O-methyl-N-{[3-({[(2,4,6- trimethylphenyl)amino]carbonyl}amino)-2- naphthalenyl]carbonyl}-L- threonine | 464 (M +H) | 0.24 |
| 278 | 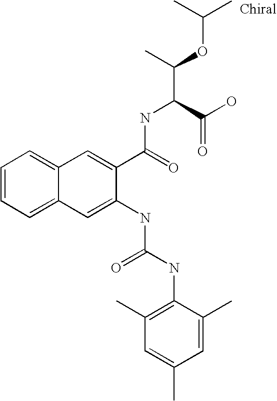 | O-(1-methylethyl)-N-{[3- ({[(2,4,6- trimethylphenyl)amino]carbonyl}amino)-2- naphthalenyl]carbonyl}-L- threonine | 492 (M +H) | 0.017 |
| 279 | 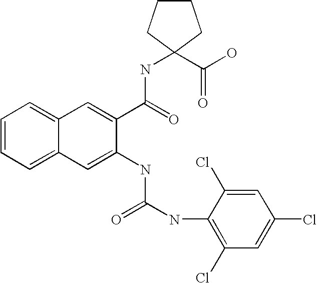 | 1-({[3-({[(2,4,6- Trichlorophenyl)amino]carbonyl}amino)-2- naphthalenyl]carbonyl}amino)cyclopentanecarboxylic acid | 521 | 3 |
| 280 | 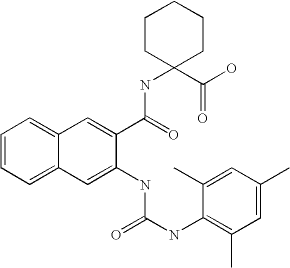 | 1-({[3-({[(2,4,6- trimethylphenyl)amino]carbonyl}amino)-2- naphthalenyl]carbonyl}amino)cyclohexanecarboxylic acid | 474 | 0.3 |
| 281 | 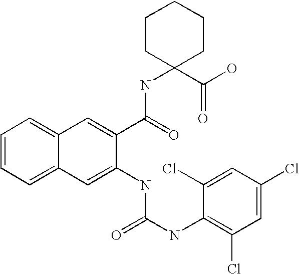 | 1-({[3-({[(2,4,6- trichlorophenyl)amino]carbonyl}amino)-2- naphthalenyl]carbonyl}amino)cyclohexanecarboxylic acid | 534 | 1.62 |
| 282 | 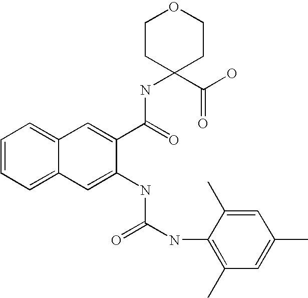 | 4-({[3-({[(2,4,6- Trimethylphenyl)amino]carbonyl}amino)-2- naphthalenyl]carbonyl}amino)tetrahydro-2H-pyran-4- carboxylic acid | 476 | 1.15 |
| 283 | 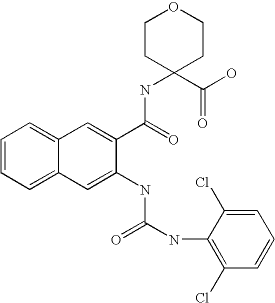 | 4-({[3-({[(2,6- Dichlorophenyl)amino]carbonyl}amino)-2- naphthalenyl]carbonyl}amino)tetrahydro-2H-pyran-4- carboxylic acid | 502 | 7.18 |
| 284 | 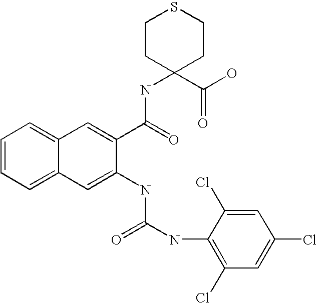 | 4-({[3-({[(2,4,6- Trichlorophenyl)amino]carbonyl}amino)-2- naphthalenyl]carbonyl}amino)tetrahydro-2H- thiopyran-4-carboxylic acid | 553 | 2.31 |
| 285 | 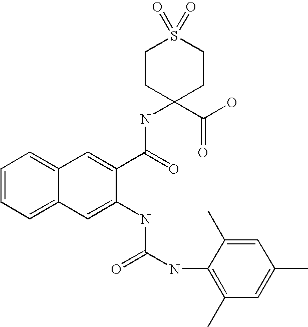 | 4-({[3-({[(2,4,6- Trimethylphenyl)amino]carbonyl}amino)-2- naphthalenyl]carbonyl}amino)tetrahydro-2H- thiopyran-4-carboxylic acid 1,1-dioxide | 524 | 3.31 |
| 286 | 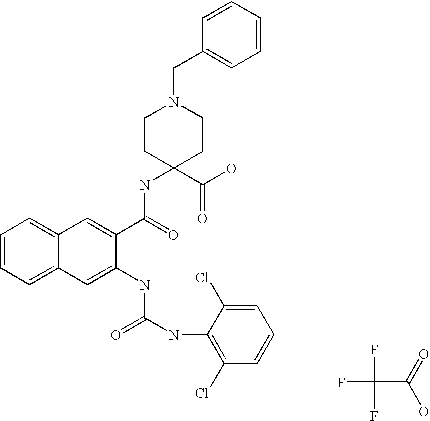 | 4-({[3-({[(2,6- Dichlorophenyl)amino]carbonyl}amino)-2- naphthalenyl]carbonyl}amino)-1-(phenylmethyl)-4- piperidinecarboxylic acid | 492 | 1.38 |
| 287 | | 1-{[(1,1- | 575 | 0.43 |
| | | Dimethylethyl)oxy]carbonyl}- | | |
| | | 4-({[3-({[(2,4,6- | | |
| | | trimethylphenyl)amino] | | |
| | | carbonyl}amino)-2- | | |
| | | naphthalenyl]carbonyl} | | |
| | | amino)-4-piperidinecarboxylic | | |
| | | acid | | |
| 288 | 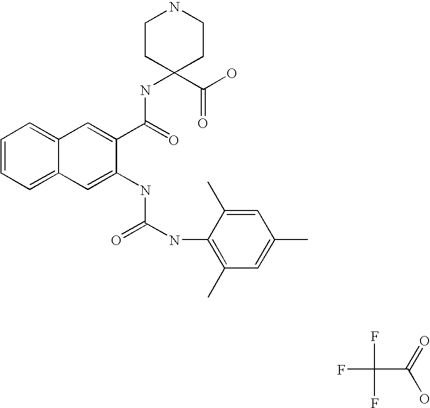 | 4-({[3-({[(2,4,6- trimethylphenyl)amino]carbonyl}amino)-2- naphthalenyl]carbonyl}amino)-4-piperidinecarboxylic acid trifluoroacetate | 475 | 6.17 |
| 289 | 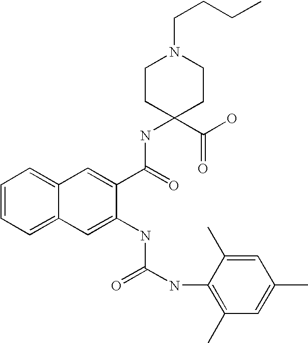 | 1-Butyl-4-({[3-({[(2,4,6- trimethylphenyl)amino]carbonyl}amino)-2- naphthalenyl]carbonyl}amino)-4-piperidinecarboxylic acid | 531 | 4.39 |
| 290 | 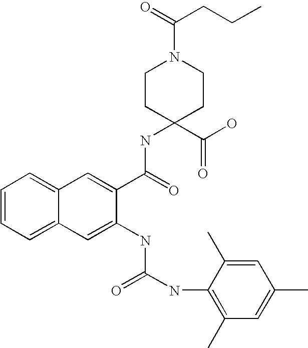 | 1-Butanoyl-4-({[3-({[(2,4,6- trimethylphenyl)amino]carbonyl}amino)-2- naphthalenyl]carbonyl}amino)-4-piperidinecarboxylic acid | 545 | 1.24 |
| 291 | 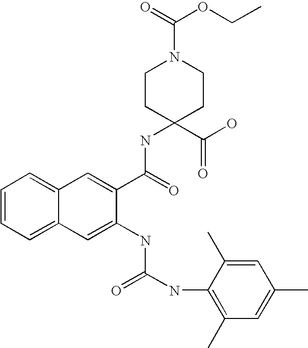 | 1-[(Ethyloxy)carbonyl]-4- ({[3-({[(2,4,6- trimethylphenyl)amino]carbonyl}amino)-2- naphthalenyl]carbonyl}amino)-4-piperidinecarboxylic acid | 547 | 0.61 |
| 292 | 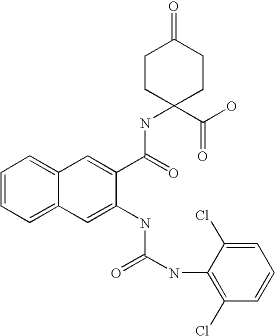 | 1-({[3-({[(2,6- dichlorophenyl)amino]carbonyl}amino)-2- naphthalenyl]carbonyl}amino)-4- oxocyclohexanecarboxylic acid | 515 | 6.59 |
| 293 | 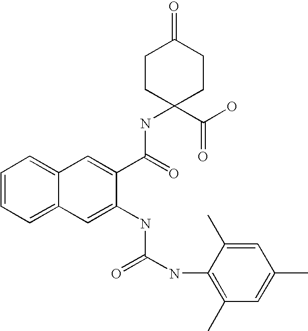 | 4-Oxo-1-({[3-({[(2,4,6- trimethylphenyl)amino]carbonyl}amino)-2- naphthalenyl]carbonyl}amino)cyclohexanecarboxylic acid | 488 | 1.67 |
| 294 | 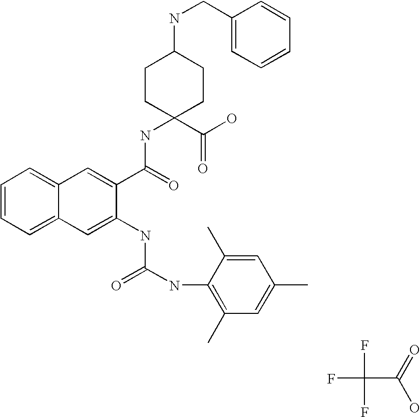 | 4-[(phenylmethyl)amino]-1- ({[3-({[(2,4,6- trimethylphenyl)amino]carbonyl}amino)-2- naphthalenyl]carbonyl}amino)cyclohexanecarboxylic acid | 579 | 2.53 |
| 295 | 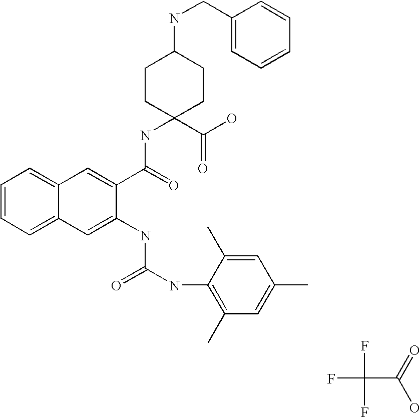 | 4-[(phenylmethyl)amino]-1- ({[3-({[(2,4,6- trimethylphenyl)amino]carbonyl}amino)-2- naphthalenyl]carbonyl}amino)cyclohexanecarboxylic acid | 579 | 5.56 |
| 296 | 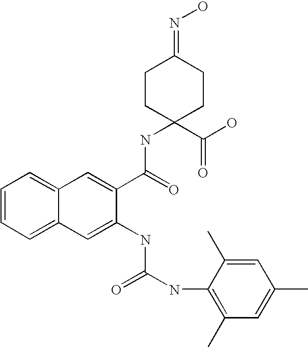 | 4-(hydroxyimino)-1-({[3- ({[(2,4,6- trimethylphenyl)amino]carbonyl}amino)-2- naphthalenyl]carbonyl}amino)cyclohexanecarboxylic acid | 503 | 0.54 |
| 297 | 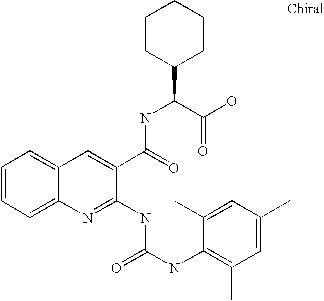 | (2S)-cyclohexyl({[2- ({[(2,4,6- trimethylphenyl)amino]carbonyl}amino)-3- quinolinyl]carbonyl}amino) ethanoic acid | 489 | 15.6 |
| 298 | 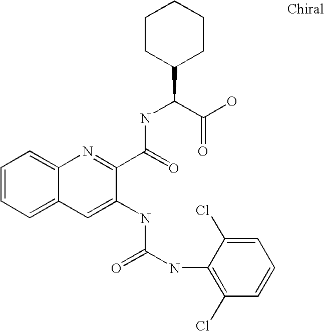 | (2S)-Cyclohexyl({[3-({[(2,6- dichlorophenyl)amino]carbonyl}amino)-2- quinolinyl]carbonyl}amino) ethanoic acid | 515 | 0.15 |
| 299 | 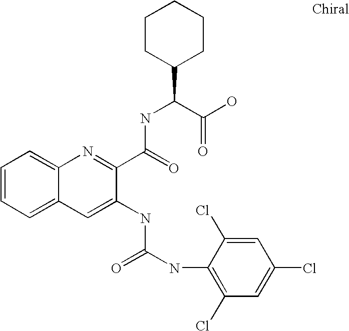 | (2S)-Cyclohexyl({[3- ({[(2,4,6- trichlorophenyl)amino]carbonyl}amino)-2- quinolinyl]carbonyl}amino) ethanoic acid | 551 | 0.48 |
| 300 | 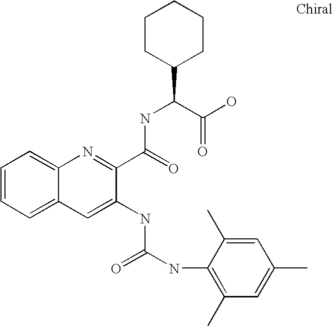 | (2S)-Cyclohexyl({[3- ({[(2,4,6- trimethylphenyl)amino]carbonyl}amino)-2- quinolinyl]carbonyl}amino) ethanoic acid | 489 | 0.027 |
| 301 | 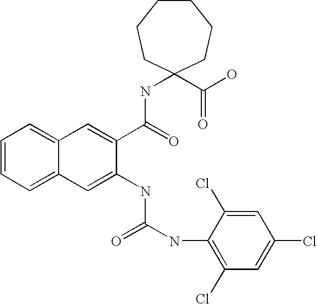 | 1-({[3-({[(2,4,6- Trichlorophenyl)amino]carbonyl}amino)-2- naphthalenyl]carbonyl}amino)cycloheptanecarboxylic acid | 549 | 0.35 |
| 302 | 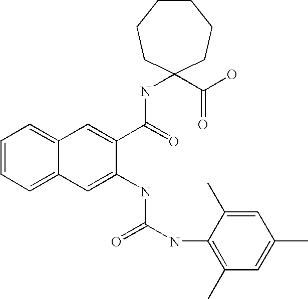 | 1-({[3-({[(2,4,6- trimethylphenyl)amino]carbonyl}amino)-2- naphthalenyl]carbonyl}amino)cycloheptanecarboxylic acid | 488 | 0.036 |
| 303 | 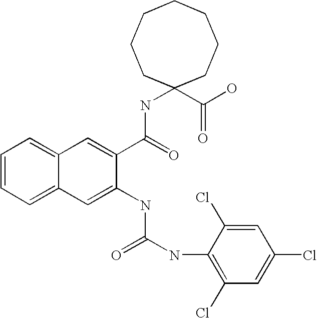 | 1-({[3-({[(2,4,6- trichlorophenyl)amino]carbonyl}amino)-2- naphthalenyl]carbonyl}amino)cyclooctanecarboxylic acid | 563 | 0.057 |
| 304 | 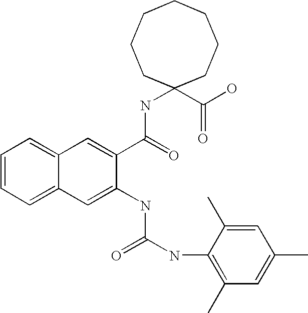 | 1-({[3-({[(2,4,6- trimethylphenyl)amino]carbonyl}amino)-2- naphthalenyl]carbonyl}amino)cyclooctanecarboxylic acid | 502 | 0.005 |
| 305 | 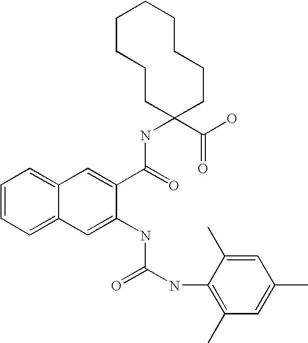 | 1-({[3-({[(2,4,6- trimethylphenyl)amino]carbonyl}amino)-2- naphthalenyl]carbonyl}amino)cyclodecanecarboxylic acid | 530 | 0.006 |
| 306 | 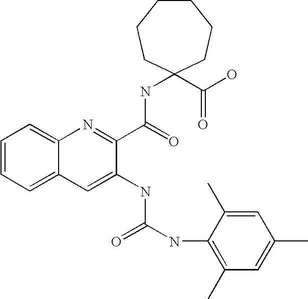 | 1-({[3-({[(2,4,6- trimethylphenyl)amino]carbonyl}amino)-2- quinolinyl]carbonyl}amino) cycloheptanecarboxylic acid | 489 | 0.011 |
| 307 | 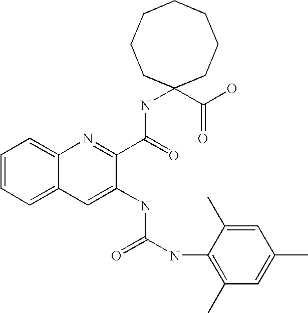 | 1-({[3-({[(2,4,6- trimethylphenyl)amino]carbonyl}amino)-2- quinolinyl]carbonyl}amino) cyclooctanecarboxylic acid | 503 | 0.002 |
| 308 | 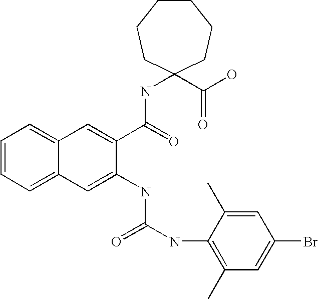 | 1-({[3-({[(4-bromo-2,6- dimethylphenyl)amino]carbonyl}amino)-2- naphthalenyl]carbonyl}amino)cycloheptanecarboxylic acid | 553 | 0.25 |
| 309 | 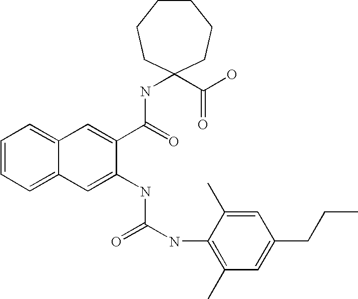 | 1-({[3-({[(2,6-dimethyl-4- propylphenyl)amino]carbonyl}amino)-2- naphthalenyl]carbonyl}amino)cycloheptanecarboxylic acid | 516 | 0.011 |
| 310 | 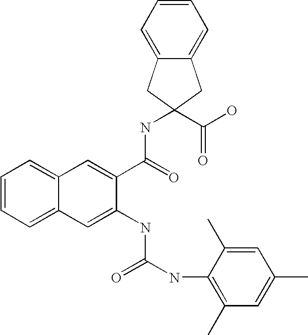 | 2-({[3-({[(2,4,6- trimethylphenyl)amino]carbonyl}amino)-2- naphthalenyl]carbonyl}amino)-2,3-dihydro-1H-indene- 2-carboxylic acid | 508 | 0.064 |
| 311 | 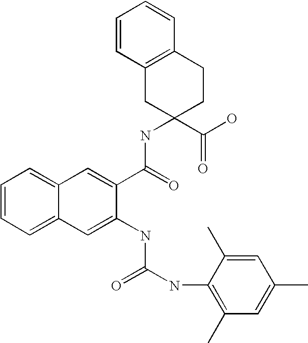 | 2-({[3-({[(2,4,6- trimethylphenyl)amino]carbonyl}amino)-2- naphthalenyl]carbonyl}amino)-1,2,3,4-tetrahydro-2- naphthalenecarboxylic acid | 522 | 0.037 |
| 312 | 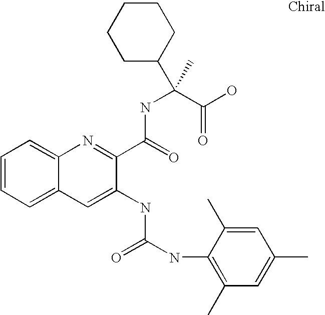 | 2-Cyclohexyl-N-{[3- ({[(2,4,6- trimethylphenyl)amino]carbonyl}amino)-2- quinolinyl]carbonyl}-D- alanine | 503 | 0.23 |
| 313 | 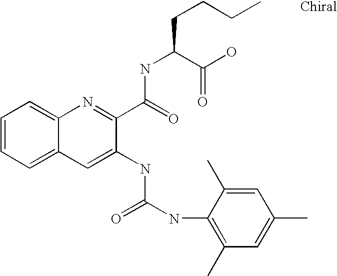 | N-{[3-({[(2,4,6- trimethylphenyl)amino]carbonyl}amino)-2- quinolinyl]carbonyl}-L- norleucine | 463 | 0.16 |
| 314 | 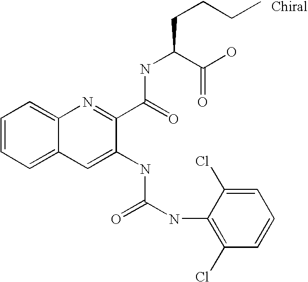 | 2-{[3-({[(2,6- dichlorophenyl)amino]carbonyl}amino)-2- quinolinyl]carbonyl}-L- norleucine | 489 | 1.8 |
| 315 | 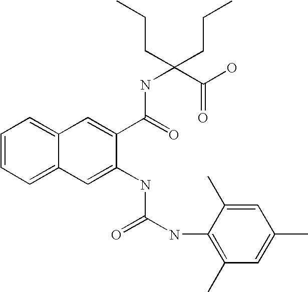 | 2-Propyl-N-{[3-({[(2,4,6- trimethylphenyl)amino]carbonyl}amino)-2- naphthalenyl]carbonyl}norvaline | 490 | 0.73 |
| 316 | 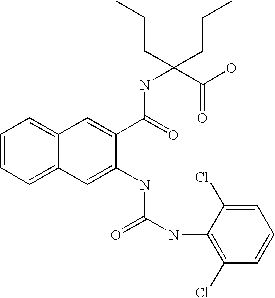 | N-{[3-({[(2,6- dichlorophenyl)amino]carbonyl}amino)-2- naphthalenyl]carbonyl}-2- propylnorvaline | 516 | 0.36 |
| 317 | 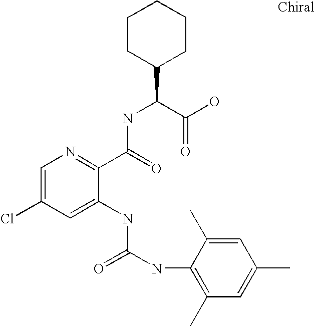 | (2S)-({[5-chloro-3-({[(2,4,6- trimethylphenyl)amino]carbonyl}amino)-2- pyridinyl]carbonyl}amino) cyclohexyl)ethanoic acid | 473 | 0.92 |
| 318 | 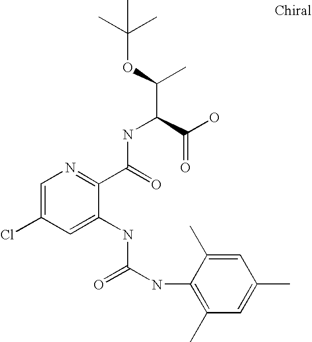 | N-{[5-chloro-3-({[(2,4,6- trimethylphenyl)amino]carbonyl}amino)-2- pyridinyl]carbonyl}-O-(1,1- dimethylethyl)-L-threonine | 491 | 4.28 |
| 319 | 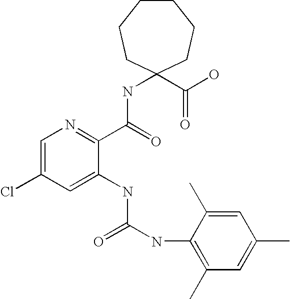 | 1-({[5-chloro-3-({[(2,4,6- trimethylphenyl)amino]carbonyl}amino)-2- pyridinyl]carbonyl}amino) cycloheptanecarboxylic acid | 473 | 0.47 |
| 320 | 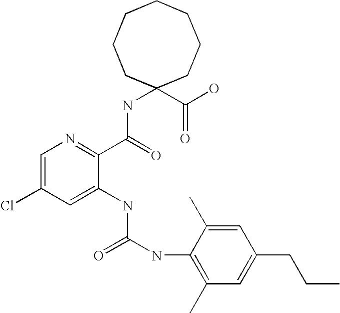 | 1-({[5-Chloro-3-({[(2,6- dimethyl-4- propylphenyl)amino]carbonyl}amino)-2- pyridinyl]carbonyl}amino) cyclooctanecarboxylic acid | 515 | 0.038 |
| 321 | 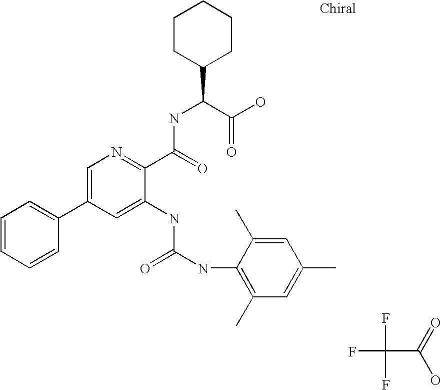 | (2S)-Cyclohexyl({[5- phenyl-3-({[(2,4,6- trimethylphenyl)amino]carbonyl}amino)-2- pyridinyl]carbonyl}amino) ethanoic acid | 515 | 0.15 |
| 322 | 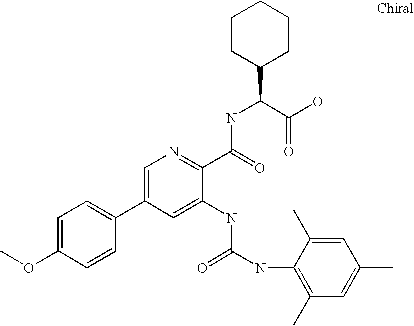 | (2S)-Cyclohexyl({[5-[4- (methyloxy)phenyl]-3- ({[(2,4,6- trimethylphenyl)amino]carbonyl}amino)-2- pyridinyl]carbonyl}amino) ethanoic acid | 545 | 0.083 |
| 323 | 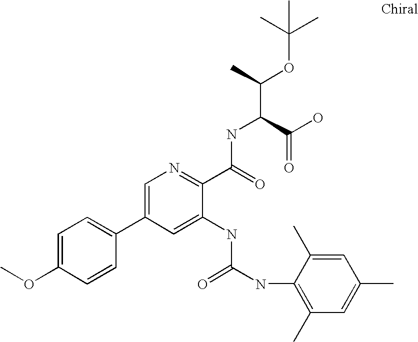 | O-(1,1-dimethylethyl)-N- {[5-[4-(methyloxy)phenyl]- 3-({[(2,4,6- trimethylphenyl)amino]carbonyl}amino)-2- pyridinyl]carbonyl}-L- threonine | 563 | 0.17 |
| 324 | 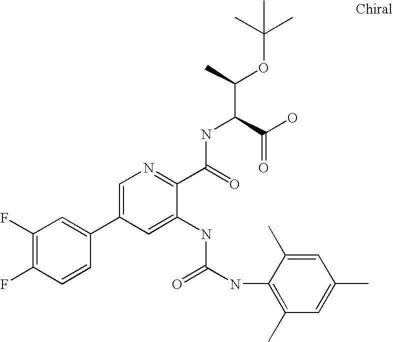 | N-{[5-(3,4-Difluorophenyl)- 3-({[(2,4,6- trimethylphenyl)amino]carbonyl}amino)-2- pyridinyl]carbonyl}-O-(1,1- dimethylethyl)-L-threonine | 569 | 0.47 |
| 325 | 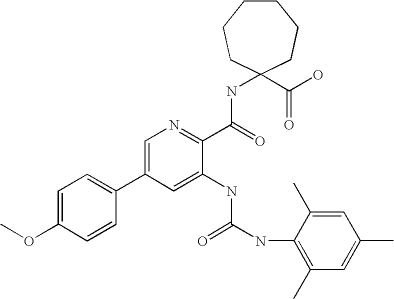 | 1-({[5-[4- (methyloxy)phenyl]-3- ({[(2,4,6- trimethylphenyl)amino]carbonyl}amino)-2- pyridinyl]carbonyl}amino) cycloheptanecarboxylic acid | 545 | 0.031 |
| 326 | 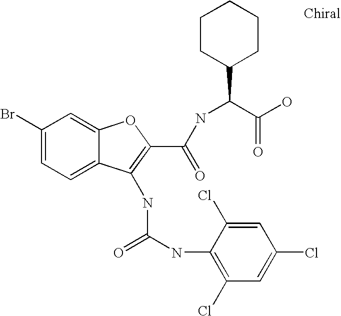 | (2S)-({[6-Bromo-3-({[(2,4,6- trichlorophenyl)amino]carbonyl}amino)-1-benzofuran- 2- yl]carbonyl}amino)(cyclohexyl) ethanoic acid | 618 | 7.39 |
| 327 | 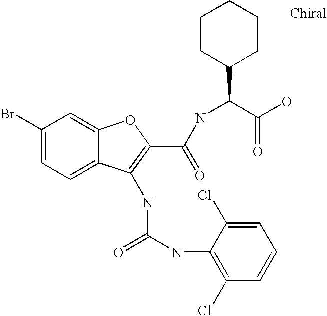 | (2S)-({[6-Bromo-3-({[(2,6- dichlorophenyl)amino]carbonyl}amino)-1-benzofuran- 2- yl]carbonyl}amino)(cyclohexyl) ethanoic acid | 584 | 9.87 |
| 328 | 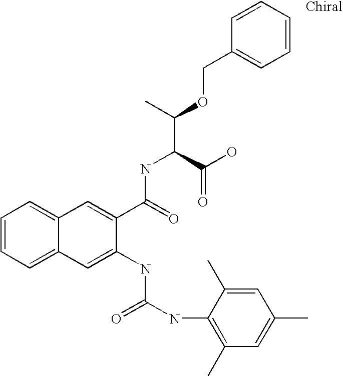 | O-(phenylmethyl)-N-{[3- ({[(2,4,6- trimethylphenyl)amino]carbonyl}amino)-2- naphthalenyl]carbonyl}-L- threonine | 539 | 0.022 |
| 329 | 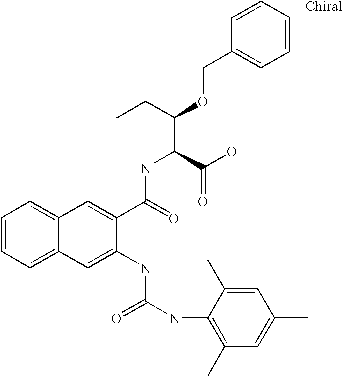 | (3R)-3- [(phenylmethyl)oxy]-N-{[3- ({[(2,4,6- trimethylphenyl)amino]carbonyl}amino)-2- naphthalenyl]carbonyl}-L- norvaline | 554 | 0.025 |
| 330 | 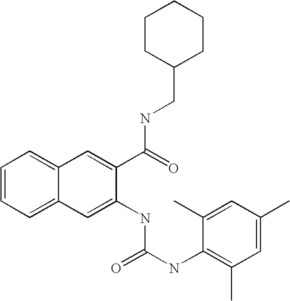 | N-(cyclohexylmethyl)-3- ({[(2,4,6- trimethylphenyl)amino]carbonyl}amino)-2- naphthalenecarboxamide | 444 | 14.4 |
| 331 | 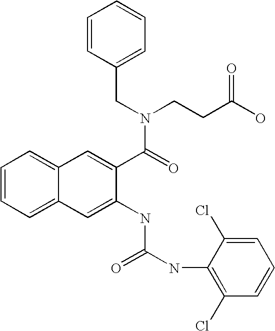 | N-{[3-({[(2,6- dichlorophenyl)amino]carbonyl}amino)-2- naphthalenyl]carbonyl}-N- (phenylmethyl)--alanine | 536 | 2.18 |
| 332 | 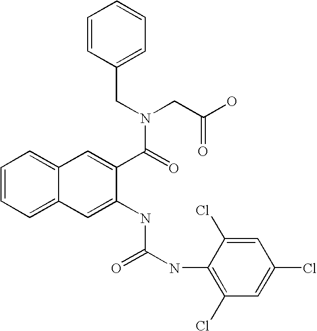 | N-(phenylmethyl)-N-{[3- ({[(2,4,6- trichlorophenyl)amino]carbonyl}amino)-2- naphthalenyl]carbonyl}glycine | 577 | 4.35 |
| 333 | 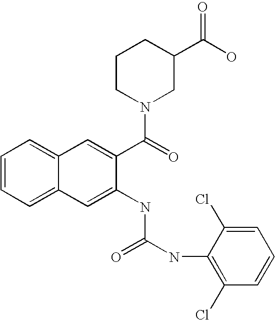 | 1-{[3-({[(2,6- Dichlorophenyl)amino]carbonyl}amino)-2- naphthalenyl]carbonyl}-3- piperidinecarboxylic acid | 486 | 9.27 |
| 334 | 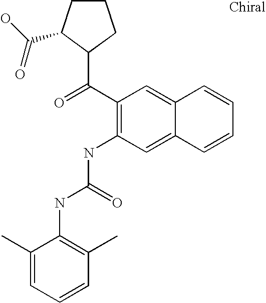 | 1-{[3-({[(2,6- dimethylphenyl)amino]carbonyl}amino)-2- naphthalenyl]carbonyl}-L- proline | 430 (M −H) | 0.42 |
| 335 | 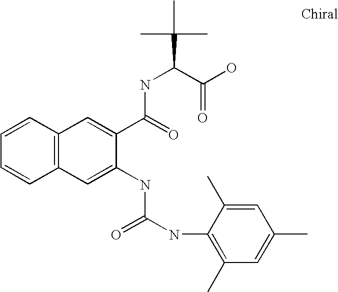 | 3-methyl-N-{[3-({[(2,4,6- trimethylphenyl)amino]carbonyl}amino)-2- naphthalenyl]carbonyl}-L- valine | 460 (M −H) | 0.3 |
| 336 | 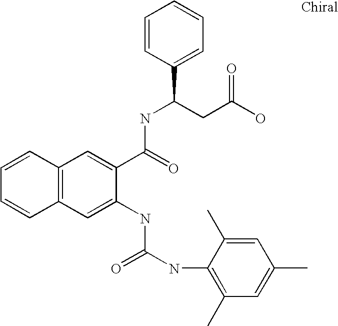 | (3R)-3-phenyl-3-({[3- ({[(2,4,6- trimethylphenyl)amino]carbonyl}amino)-2- naphthalenyl]carbonyl}amino)propanoic acid | 494 (M −H) | 0.61 |
| 337 | 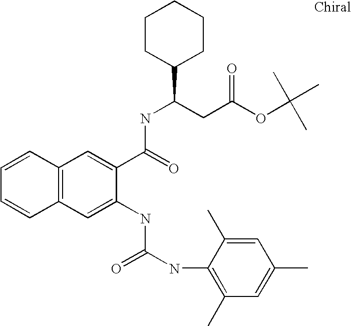 | 1,1-dimethylethyl (3R)-3- cyclohexyl-3-({[3-({[(2,4,6- trimethylphenyl)amino]carbonyl}amino)-2- naphthalenyl]carbonyl}amino)propanoate | 556 (M −H) | 5.26 |
| 338 | 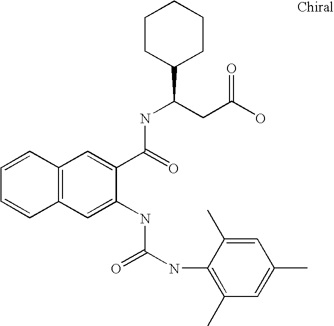 | (3R)-3-cyclohexyl-3-({[3- ({[(2,4,6- trimethylphenyl)amino]carbonyl}amino)-2- naphthalenyl]carbonyl}amino)propanoic acid | 500 (M −H) | 2.13 |
| 339 | 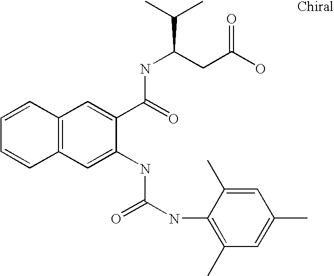 | (3R)-4-methyl-3-({[3- ({[(2,4,6- trimethylphenyl)amino]carbonyl}amino)-2- naphthalenyl]carbonyl}amino)pentanoic acid | 460 (M −H) | 1.56 |
| 340 | 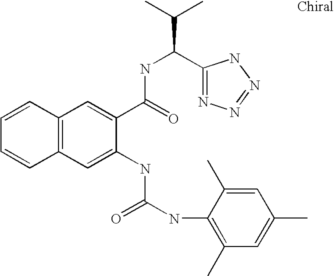 | N-[(1S)-2-methyl-1-(1H- tetrazol-5-yl)propyl]-3- ({[(2,4,6- trimethylphenyl)amino]carbonyl}amino)-2- naphthalenecarboxamide | 470 (M −H) | 0.34 |
| 341 | 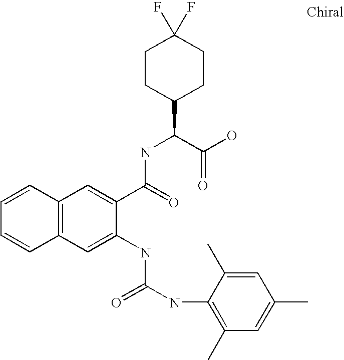 | (2S)-(4,4- difluorocyclohexyl)({[3- ({[(2,4,6- trimethylphenyl)amino]carbonyl}amino)-2- naphthalenyl]carbonyl}amino)ethanoic acid | 522 (M −H) | 0.032 |
| 342 | 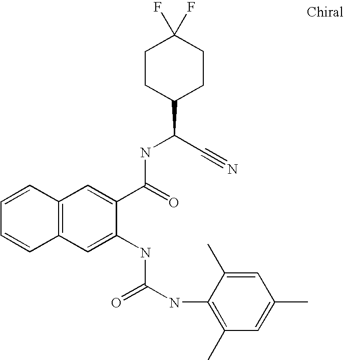 | N-[(S)-cyano(4,4- difluorocyclohexyl)methyl]- 3-({[(2,4,6- trimethylphenyl)amino]carbonyl}amino)-2- naphthalenecarboxamide | 503 (M −H) | 8.6 |
| 343 | 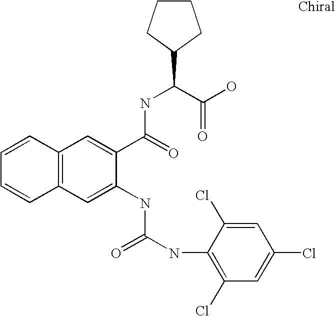 | (2S)-cyclopentyl({[3- ({[(2,4,6- trichlorophenyl)amino]carbonyl}amino)-2- naphthalenyl]carbonyl}amino)ethanoic acid | 533 (M −H) | 0.13 |
| 344 | 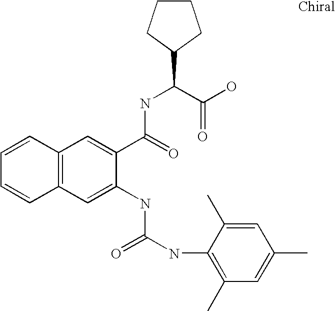 | (2S)-cyclopentyl({[3- ({[(2,4,6- trimethylphenyl)amino]carbonyl}amino)-2- naphthalenyl]carbonyl}amino)ethanoic acid | 472 (M −H) | 0.013 |
| 345 | 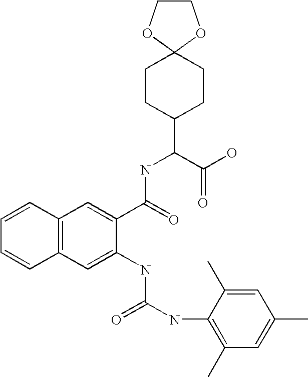 | 1,4-dioxaspiro[4.5]dec-8- yl({[3-({[(2,4,6- trimethylphenyl)amino]carbonyl}amino)-2- naphthalenyl]carbonyl}amino)acetic acid | 544 (M −H) | 0.034 |
| 346 | 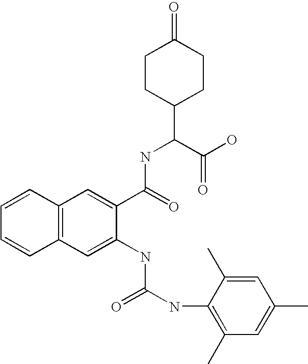 | (4-oxocyclohexyl)({[3- ({[(2,4,6- trimethylphenyl)amino]carbonyl}amino)-2- naphthalenyl]carbonyl}amino)acetic acid | 500 (M −H) | 0.17 |
| 347 | 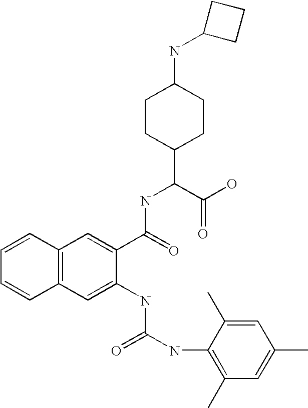 | (cis and trans)-[4- (cyclobutylamino)cyclohexyl]({[3-({[(2,4,6- trimethylphenyl)amino]carbonyl}amino)-2- naphthalenyl]carbonyl}amino)acetic acid | 577 (M +H) APCI | 0.81 |
| 348 | 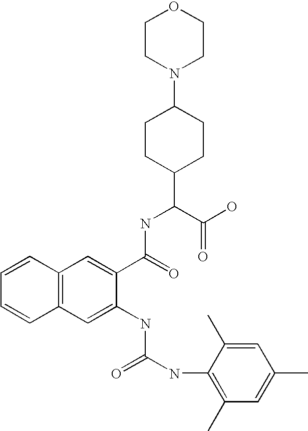 | (cis and trans)-[4-(4- morpholinyl)cyclohexyl]({[3- ({[(2,4,6- trimethylphenyl)amino]carbonyl}amino)-2- naphthalenyl]carbonyl}amino)acetic acid | 572 (M −H) | 1.86 |
| 349 | 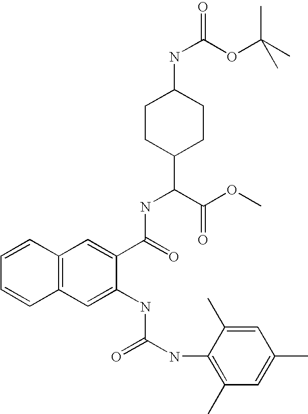 | (cis and trans)-methyl [4- ({[(1,1- dimethylethyl)oxy]carbonyl}amino)cyclohexyl]({[3- ({[(2,4,6- trimethylphenyl)amino]carbonyl}amino)-2- naphthalenyl]carbonyl}amino)acetate | 615 (M −H) | 6.89 |
| 350 | 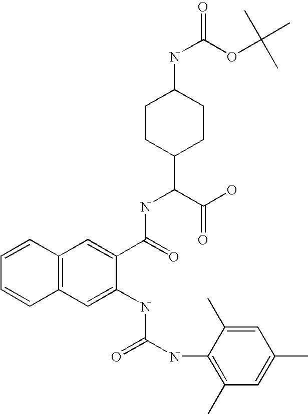 | (cis and trans)-[4-({[(1,1- dimethylethyl)oxy]carbonyl}amino)cyclohexyl]({[3- ({[(2,4,6- trimethylphenyl)amino]carbonyl}amino)-2- naphthalenyl]carbonyl}amino)acetic acid | 601 (M −H) | 0.044 |
| 351 | 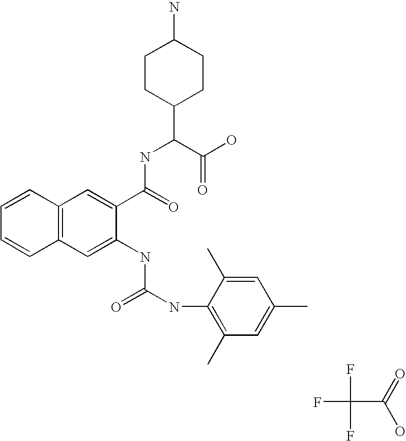 | (cis and trans)-(4- aminocyclohexyl)({[3- ({[(2,4,6- trimethylphenyl)amino]carbonyl}amino)-2- naphthalenyl]carbonyl}amino)acetic acid trifluoroacetate | 501 (M −H) | 2.37 |
| 352 | 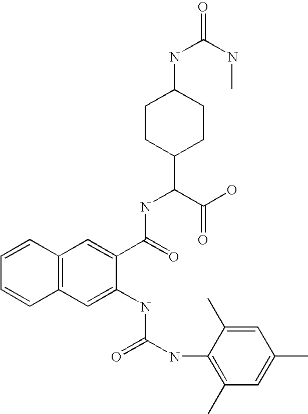 | (cis and trans)-(4- {[(methylamino)carbonyl]amino}cyclohexyl)({[3- ({[(2,4,6- trimethylphenyl)amino]carbonyl}amino)-2- naphthalenyl]carbonyl}amino)acetic acid | 558 (M −H) | 0.1 |
| 353 | 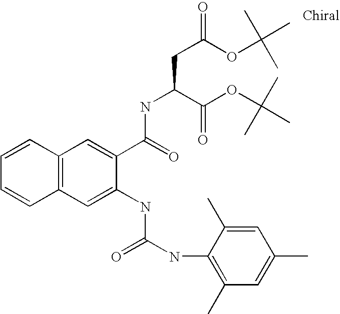 | bis(1,1-dimethylethyl) N- {[3-({[(2,4,6- trimethylphenyl)amino]carbonyl}amino)-2- naphthalenyl]carbonyl}-L- aspartate | 574 (M −H) | 5.08 |
| 354 | 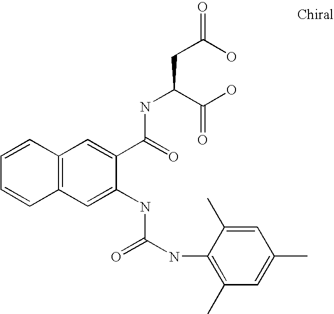 | N-{[3-({[(2,4,6- trimethylphenyl)amino]carbonyl}amino)-2- naphthalenyl]carbonyl}-L- aspartic acid | 462 (M −H) | 0.061 |
| 355 | 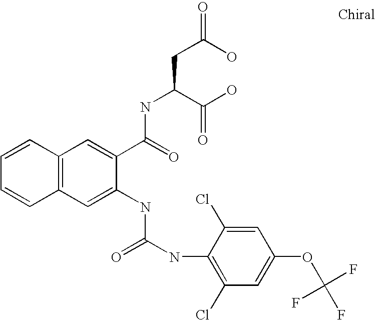 | N-[(3-{[({2,6-dichloro-4- [(trifluoromethyl)oxy]phenyl}amino)carbonyl]amino}-2- naphthalenyl)carbonyl]-L- aspartic acid | 572 (M −H) | 0.014 |
| 356 | 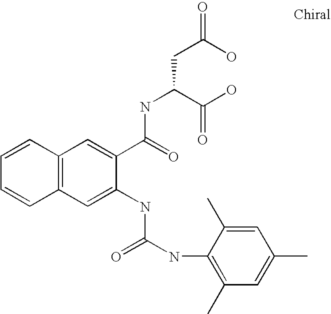 | N-{[3-({[(2,4,6- trimethylphenyl)amino]carbonyl}amino)-2- naphthalenyl]carbonyl}-D- aspartic acid | 462 (M −H) | 0.1 |
| 357 | 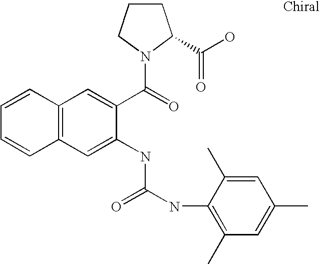 | 1-{[3-]{[(2,4,6- trimethylphenyl)amino]carbonyl}amino)-2- naphthalenyl]carbonyl}-D- proline | 444 (M −H) | 2.47 |
| 358 | 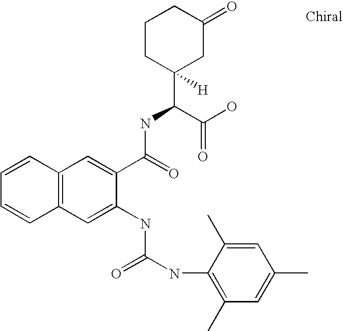 | (2S)-[(1S)-3- oxocyclohexyl]({[3- ({[(2,4,6- trimethylphenyl)amino]carbonyl}amino)-2- naphthalenyl]carbonyl}amino)ethanoic acid | 500 (M −H) | 0.075 |
| 359 | 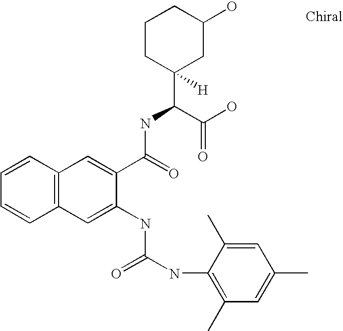 | (2S)-[(1S)-3- hydroxycyclohexyl]({[3- ({[(2,4,6- trimethylphenyl)amino]carbonyl}amino)-2- naphthalenyl]carbonyl}amino)ethanoic acid | 502 (M −H) | 0.006 |
| 360 | 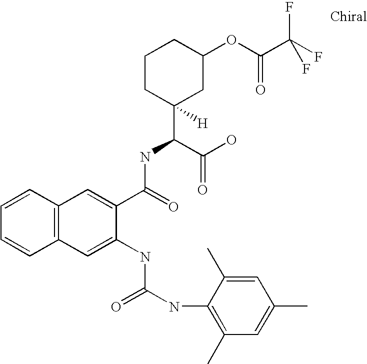 | (2S)-{(1S)-3- [(trifluoroacetyl)oxy]cyclohexyl}({[3-({[(2,4,6- trimethylphenyl)amino]carbonyl}amino-2- naphthalenyl]carbonyl}amino)ethanoic acid | 598 (M −H) | 0.011 |
| 361 | 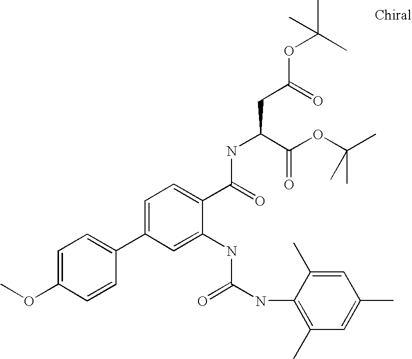 | bis(1,1-dimethylethyl) N- {[4′-(methyloxy)-3-({[(2,4,6- trimethylphenyl)amino]carbonyl}amino)-4- biphenylyl]carbonyl}-L- aspartate | 630 (M −H) APCI | 6.07 |
| 362 | 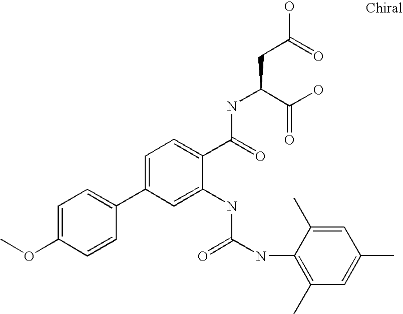 | N-{[4′-(methyloxy)-3- ({[(2,4,6- trimethylphenyl)amino]carbonyl}amino)-4- biphenylyl]carbonyl}-L- aspartic acid | 518 (M −H) | 0.006 |
| 363 | 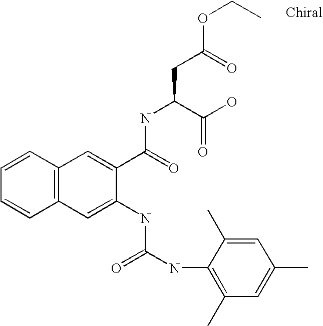 | (2S)-4-(ethyloxy)-4-oxo-2- ({[3-({[(2,4,6- trimethylphenyl)amino]carbonyl}amino)-2- naphthalenyl]carbonyl}amino)butanoic acid | 490 (M −H) | 0.36 |
| 364 | 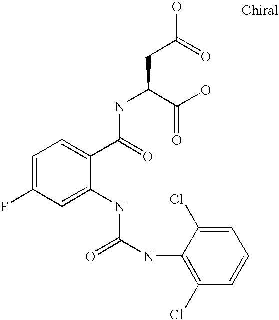 | N-{[2-({[(2,6- dichlorophenyl)amino]carbonyl}amino)-4- fluorophenyl]carbonyl}-L- aspartic acid | 456 (M −H) | 1.28 |
| 365 | 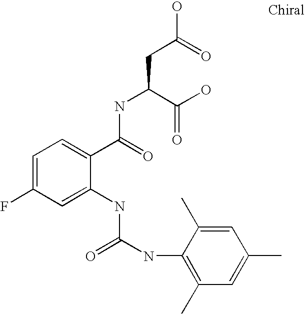 | N-{[4-fluoro-2-({[(2,4,6- trimethylphenyl)amino]carbonyl}amino)phenyl]carbonyll}-L-aspartic acid | 430 (M −H) | 1.68 |
| 366 | 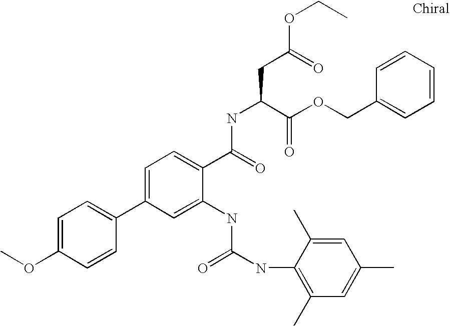 | 4-ethyl 1-(phenylmethyl) N- {[4′-(methyloxy)-3-({[(2,4,6- trimethylphenyl)amino]carbonyl}amino)-4- biphenylyl]carbonyl}-L- aspartate | 638 (M +H) | 11.1 |
| 367 | 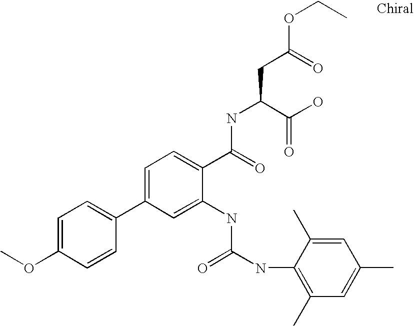 | (2S)-4-(ethyloxy)-2-({[4′- (methyloxy)-3-({[(2,4,6- trimethylphenyl)amino]carbonyl}amino)-4- biphenylyl]carbonyl}amino)- 4-oxobutanoic acid | 546 (M −H) | 0.64 |
| 368 | 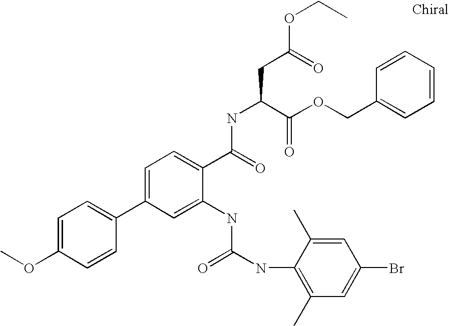 | 4-ethyl 1-(phenylmethyl) N- {[3-({[(4-bromo-2,6- dimethylphenyl)amino]carbonyl}amino)-4′- (methyloxy)-4- biphenylyl]carbonyl}-L- aspartate | 703 (M +H) | 5.58 |
| 369 | 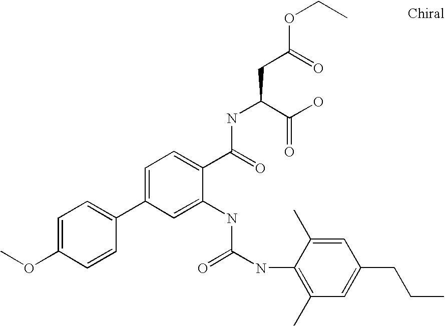 | (2S)-2-({[3-({[(2,6-dimethyl-4- propylphenyl)amino]carbonyl}amino)-4′-(methyloxy)-4- biphenylyl]carbonyl}amino)- 4-(ethyloxy)-4- oxobutanoic acid | 574 (M −H) APCI | 0.094 |
| 370 | 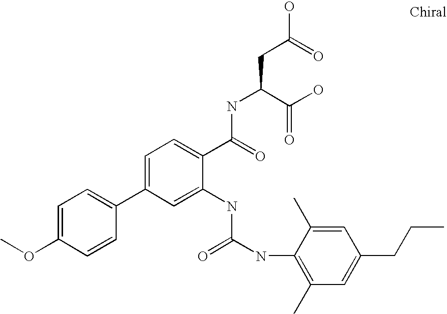 | N-{[3-({[(2,6-dimethyl-4- propylphenyl)amino]carbonyl}amino)-4′-(methyloxy)-4- biphenylyl]carbonyl}-L- aspartic acid | 547 (M −H) APCI | 0.002 |
| 371 | 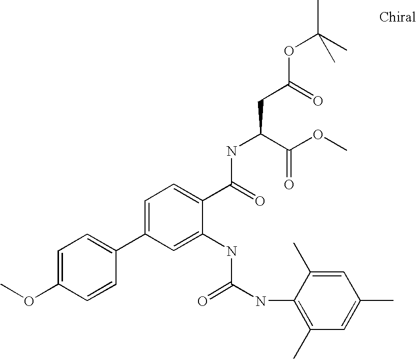 | 4-(1,1-dimethylethyl) 1- methyl N-{[4′-(methyloxy)- 3-({[(2,4,6- trimethylphenyl)amino]carbonyl}amino)-4- biphenylyl]carbonyl}-L- aspartate | 588 (M −H) | 13.54 |
| 372 | 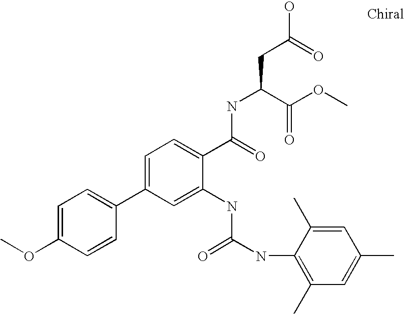 | (3S)-4-(methyloxy)-3-({[4′- (methyloxy)-3-({[(2,4,6- trimethylphenyl)amino]carbonyl}amino)-4- biphenylyl]carbonyl}amino)- 4-oxobutanoic acid | 532 (M −H) | 0.99 |
| 373 | 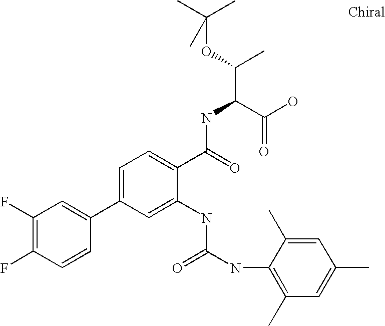 | N{[3′,4′-difluoro-3-({[(2,4,6- trimethylphenyl)amino]carbonyl}amino)-4- biphenylyl]carbonyl}-O- (1,1-dimethylethyl)-L- threonine | 566 (M −H) | 0.005 |
| 374 | 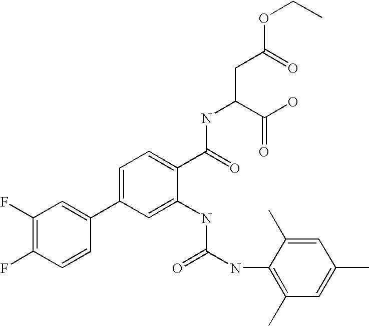 | (2S)-2-({[3′,4′-difluoro-3- ({[(2,4,6- trimethylphenyl)amino]carbonyl}amino)-4- biphenylyl]carbonyl}amino)- 4-(ethyloxy)-4- oxobutanoic acid | 554 (M +H) APCI | 0.82 |
| 375 | 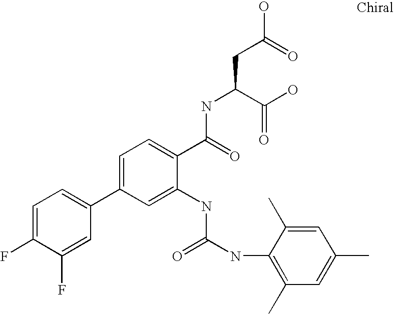 | N-{[3′,4′-difluoro-3-({[(2,4,6- trimethylphenyl)amino]carbonyl}amino)-4- biphenylyl]carbonyl}-L- aspartic acid | 524 (M −H) APCI | 0.043 |
| 376 | 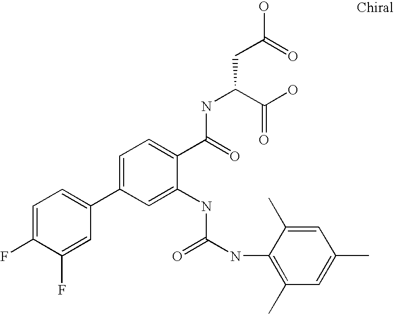 | N-{[3′,4′-difluoro-3-({[(2,4,6- trimethylphenyl)amino]carbonyl}amino)-4- biphenylyl]carbonyl}-D- aspartic acid | 524 (M −H) APCI | 0.11 |
| 377 | 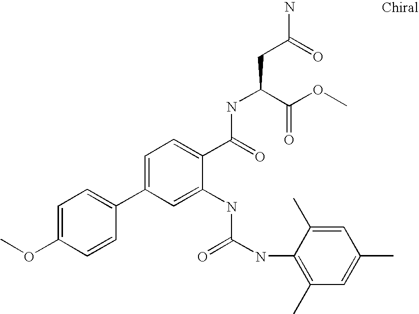 | methyl N²-{[4′-(methyloxy)- 3-({[(2,4,6- trimethylphenyl)amino]carbonyl}amino)-4- biphenylyl]carbonyl}-L- asparaginate | 531 (M −H) APCI | 9.13 |
| 378 | 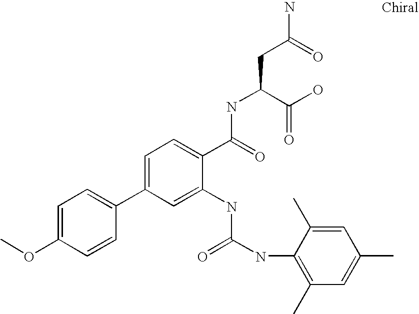 | N²-{[4′-(methyloxy)-3- ({[(2,4,6- trimethylphenyl)amino]carbonyl}amino)-4- biphenylyl]carbonyl}-L- asparagine | 517 (M −H) APCI | 0.1 |
| 379 | 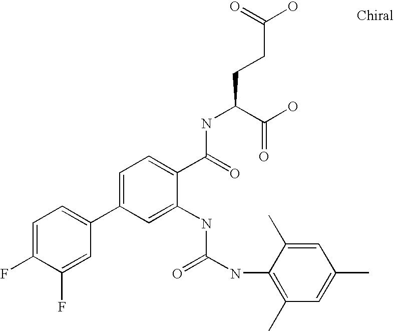 | N-{[3′,4′-difluoro-3-({[(2,4,6- trimethylphenyl)amino]carbonyl}amino)-4- biphenylyl]carbonyl}-L- glutamic acid | 538 (M −H) APCI | 0.084 |
| 380 | 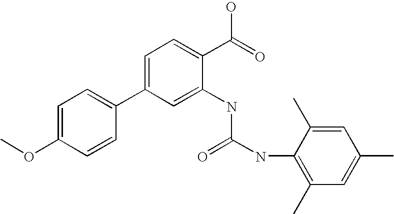 | 4′-(methyloxy)-3-({[(2,4,6- trimethylphenyl)amino]carbonyl}amino)-4- biphenylcarboxylic acid | 403 (M −H) | 1.99 |
| 381 | 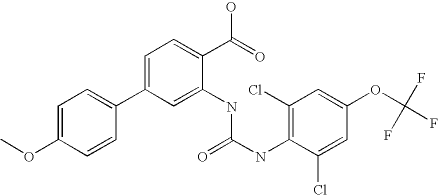 | 3-{[({2,6-dichloro-4- [(trifluoromethyl)oxy]phenyl}amino)carbonyl]amino}-4′- (methyloxy)-4- biphenylcarboxylic acid | 517 (M +H) | 3.42 |
| 382 | 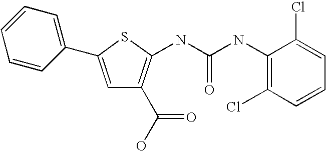 | 2-({[(2,6- dichlorophenyl)amino]carbonyl}amino)-5-phenyl-3- thiophenecarboxylic acid | 407 | 1.17 |
| 383 | 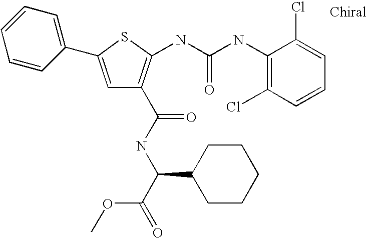 | methyl (2S)-cyclohexyl({[2- ({[(2,6- dichlorophenyl)amino]carbonyl}amino)-5-phenyl-3- thieny]carbonyl}amino) ethanoate | 560 | 2.37 |
| 384 | 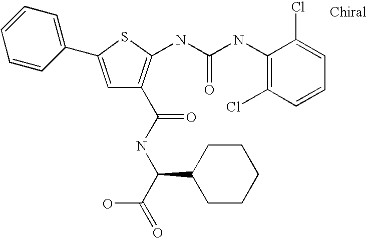 | (2S)-cyclohexyl({[2-({[(2,6- dichlorophenyl)amino]carbonyl}amino)-5-phenyl-3- thienyl]carbonyl}amino) ethanoic acid | 555 | 0.23 |
| 385 | 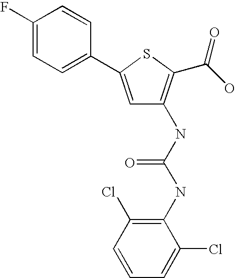 | 3-({[(2,6- dichlorophenyl)amino]carbonyl}amino)-5-(4- fluorophenyl)-2- thiophenecarboxylic acid | 425 | 1.14 |
| 386 | 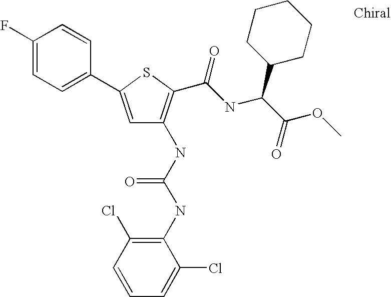 | methyl (2S)-cyclohexyl({[3- ({[(2,6- dichlorophenyl)amino]carbonyl}amino)-5-(4- fluorophenyl)-2- thienyl]carbonyl}amino) ethanoate | 578 | 4.3 |
| 387 | 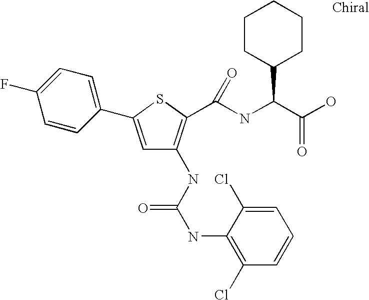 | (2S)-cyclohexyl({[3-({[(2,6- dichlorophenyl)amino]carbonyl}amino)-5-(4- fluorophenyl)-2- thienyl]carbonyl}amino) ethanoic acid | 564 | 0.43 |
| 388 | 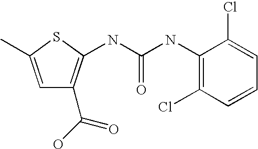 | 2-({[(2,6- dichlorophenyl)amino]carbonyl}amino)-5-methyl-3- thiophenecarboxylic acid | 345 | 19.69 |
| 389 | 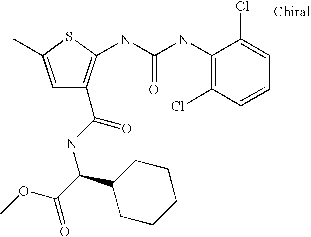 | methyl (2S)-cyclohexyl({[2- ({[(2,6- dichlorophenyl)amino]carbonyl}amino)-5-methyl-3- thienyl]carbonyl}amino) ethanoate | 498 | 15.6 |
| 390 | 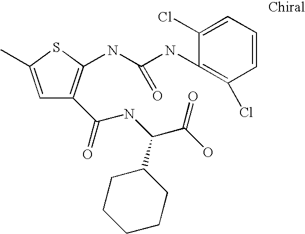 | (2S)-cyclohexyl({[2-({[(2,6- dichlorophenyl)amino]carbonyl}amino)-5-methyl-3- thienyl]carbonyl}amino) ethanoic acid | 484 | 17.51 |
| 391 | 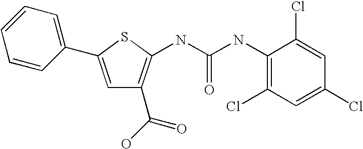 | 5-phenyl-2-({[(2,4,6- trichlorophenyl)amino]carbonyl}amino)-3- thiophenecarboxylic acid | 441 | 6.78 |
| 392 | 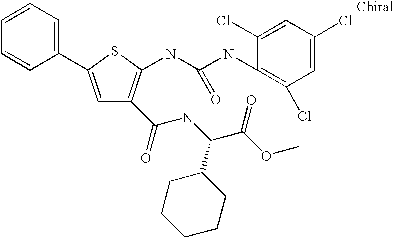 | methyl (2S)-cyclohexyl({[5- phenyl-2-({[(2,4,6- trichlorophenyl)amino]carbonyl}amino)-3- thienyl]carbonyl}amino) ethanoate | 594 | 1.32 |
| 393 | 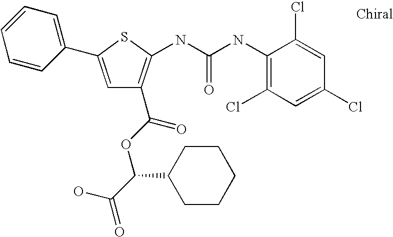 | (2R)-cyclohexyl({[5-phenyl- 2-({[(2,4,6- trichlorophenyl)amino]carbonyl}amino)-3- thienyl]carbonyl}amino) ethanoic acid | 580 | 0.84 |
| 394 | 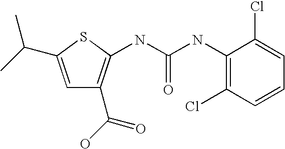 | 2-({[(2,6- dichlorophenyl)amino]carbonyl}amino)-5-(1- methylethyl)-3- thiophenecarboxylic acid | 373 | 19.26 |
| 395 | 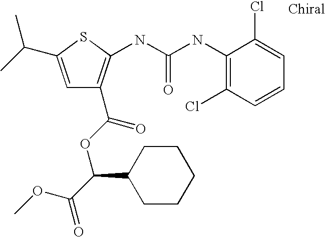 | methyl (2S)-cyclohexyl({[2- ({[(2,6- dichlorophenyl)amino]arbonyl}amino)-5-(1- methylethyl)-3- thienyl]carbonyl}amino) ethanoate | 526 | 4.49 |
| 396 | 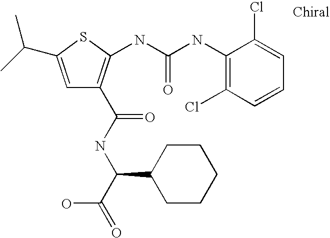 | (2S)-cyclohexyl({[2-({[(2,6- dichlorophenyl)amino]carbonyl}amino)-5-(1- methylethyl)-3- thienyl]carbonyl}amino) ethanoic acid | 580 | 1.75 |
| 397 | 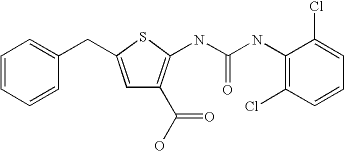 | 2-({[(2,6- dichlorophenyl)amino]carbonyl}amino)-5- (phenylmethyl)-3- thiophenecarboxylic acid | 421 | 14.28 |
| 398 | 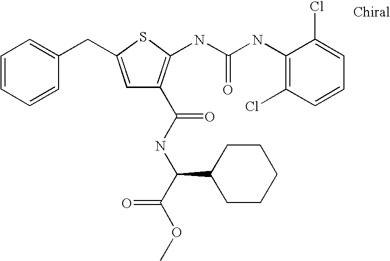 | methyl (2S)-cyclohexyl({[2- ({[(2,6- dichlorophenyl)amino]carbonyl}amino)-5- (phenylmethyl)-3- phenyl]carbonyl}amino) ethanoate | 575 | 3.06 |
| 399 | 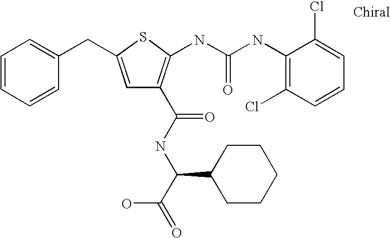 | (2S)-cyclohexyl({[2-({[(2,6- dichlorophenyl)amino]carbonyl}amino)-5- (phenylmethyl)-3- thienyl]carbonyl}amino) ethanoic acid | 561 | 6.7 |
| 400 | 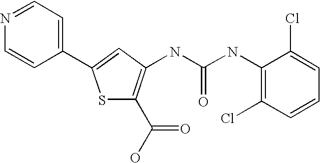 | 3-({[(2,6- dichlorophenyl)amino]carbonyl}amino)-5-(4-pyridinyl)- 2-thiophenecarboxylic acid | 408 | 1.83 |
| 401 | 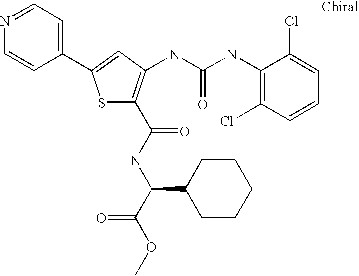 | methyl (2S)-cyclohexyl({[3- ({[(2,6- dichlorophenyl)amino]carbonyl}amino)-5-(4-pyridinyl)- 2- thienyl]carbonyl}amino) ethanoate | 562 | 14.88 |
| 402 | 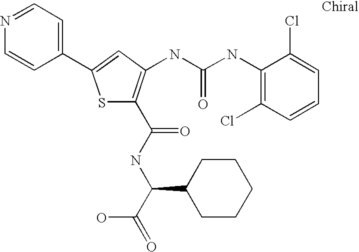 | (2S)-cyclohexyl({[3-({[(2,6- dichlorophenyl)amino]carbonyl}amino)-5-(4-pyridinyl)- 2- thienyl]carbonyl}amino) ethanoic acid | 548 | 0.17 |
| 403 | 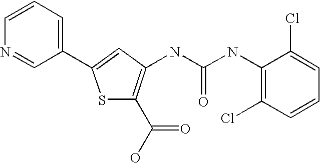 | 3-({[(2,6- dichlorophenyl)amino]carbonyl}amino)-5-(3-pyridinyl)- 2-thiophenecarboxyic acid | 408 | 4.22 |
| 404 | 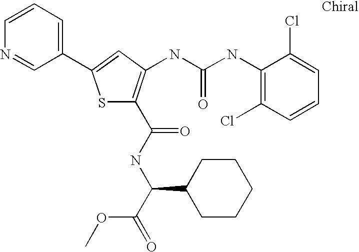 | methyl (2S)-cyclohexyl({[3- ({[(2,6- dichlorophenyl)amino]carbonyl}amino)-5-(3-pyridinyl)- 2- thienyl]carbonyl}amino) ethanoate | 562 | 13.18 |
| 405 | 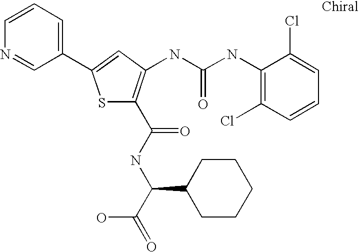 | (2S)-cyclohexyl({[3-({[(2,6- dichlorophenyl)amino]carbonyl}amino)-5-(3-pyridinyl)- 2- thienyl]carbonyl}amino) ethanoic acid | 548 | 0.23 |
| 406 | 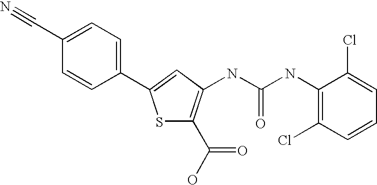 | 5-(4-cyanophenyl)-3- ({[(2,6- dichlorophenyl)amino]carbonyl}amino)-2- thiophenecarboxylic acid | 432 | 1.11 |
| 407 | 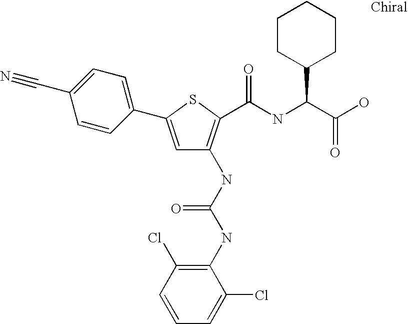 | (2S)-({[5-(4-cyanophenyl)- 3-({[(2,6- dichlorophenyl)amino]carbonyl}amino)-2- thienyl]carbonyl}amino) cyclohexyl)ethanoic acid | 571 | 0.37 |
| 408 | 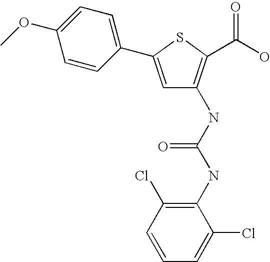 | 3-({[(2,6- dichlorophenyl)amino]carbonyl}amino)-5-[4- (methyloxy)phenyl]-2- thiophenecarboxylic acid | 437 | 1.15 |
| 409 | 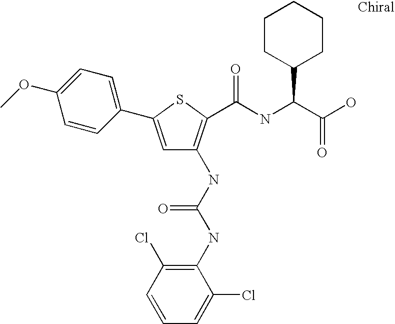 | (2S)-cyclohexyl[({3-({[(2,6- dichlorophenyl)amino]carbonyl}amino)-5-[4- (methyloxy)phenyl]-2- thienyl}carbonyl)amino]ethanoic acid | 576 | 0.093 |
| 410 | 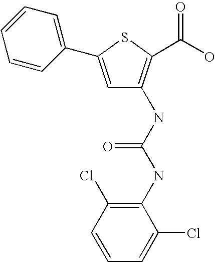 | 3-({[(2,6- dichlorophenyl)amino]carbonyl}amino)-5-phenyl-2- thiophenecarboxylic acid | 407 | 0.89 |
| 411 | 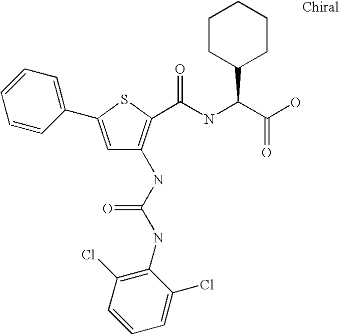 | (2S)-cyclohexyl({[3-({[(2,6- dichlorophenyl)amino]carbonyl}amino)-5-phenyl-2- thienyl]carbonyl}amino) ethanoic acid | 546 | 0.12 |
| 412 | 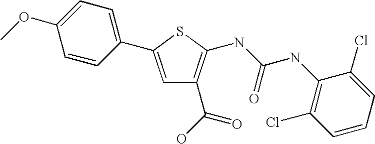 | 2-({[(2,6- dichlorophenyl)amino]carbonyl}amino)-5-[4- (methyloxy)phenyl]-3- thiophenecarboxylic acid | 437 | 1.04 |
| 413 | 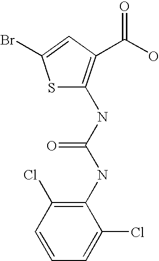 | 5-bromo-2-({[(2,6- dichlorophenyl)amino]carbonyl}amino)-3- thiophenecarboxylic acid | 410 | 10.6 |
| 414 | 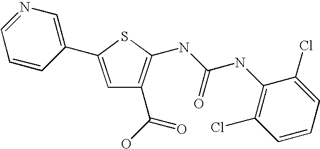 | 2-({[(2,6- dichlorophenyl)amino]carbonyl}amino)-5-(3-pyridinyl)- 3-thiophenecarboxylic acid | 408 | 2.72 |
| 415 | 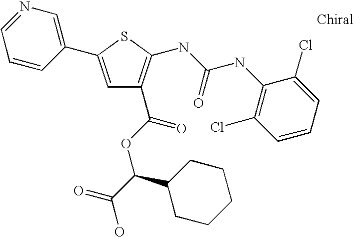 | (2S)-cyclohexyl({[2-({[(2,6- dichlorophenyl)amino]carbonyl}amino)-5-(3-pyridinyl)- 3- thienyl]carbonyl}amino) ethanoic acid | 546 | 0.77 |
| 416 | 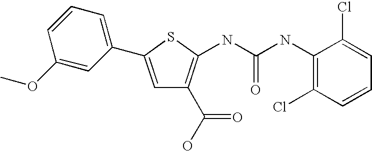 | 2-({[(2,6- dichlorophenyl)amino]carbonyl}amino)-5-[3- (methyloxy)phenyl]-3- thiophenecarboxylic acid | 437 | 4.13 |
| 417 | 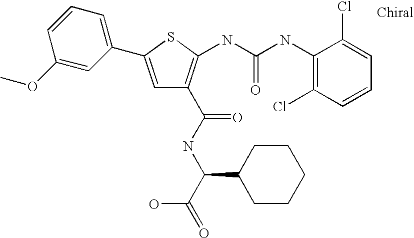 | (2S)-cyclohexyl[({2-({[(2,6- dichlorophenyl)amino]carbonyl}amino)-5-[3- (methyloxy)phenyl]-3- thienyl}carbonyl)amino]ethanoic acid | 576 | 0.36 |
| 418 | 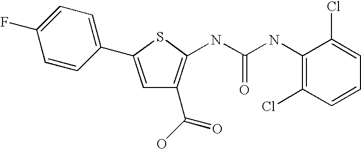 | 2-({[(2,6- dichlorophenyl)amino]carbonyl}amino)-5-(4- fluorophenyl)-3- thiophenecarboxylic acid | 425 | 2.97 |
| 419 | 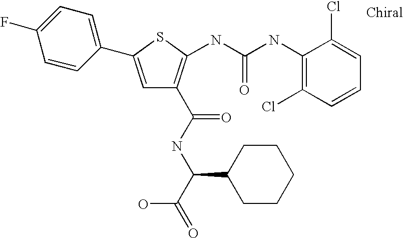 | (2S)-cyclohexyl({[2-({[(2,6- dichlorophenyl)amino]carbonyl}amino)-5-(4- fluorophenyl)-3- thienyl]carbonyl}amino) ethanoic acid | 564 | 0.6 |
| 420 | 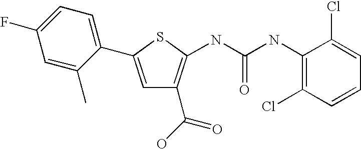 | 2-({[(2,6- dichlorophenyl)amino]carbonyl}amino)-5-(4-fluoro-2- methylphenyl)-3- thiophenecarboxylic acid | 439 | 5.64 |
| 421 | 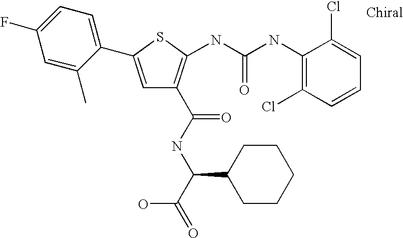 | (2S)-cyclohexyl({[2-({[(2,6- dichlorophenyl)amino]carbonyl}amino)-5-(4-fluoro-2- methylphenyl)-3- thienyl]carbonyl}amino) ethanoic acid | 564 | 1.39 |
| 422 | 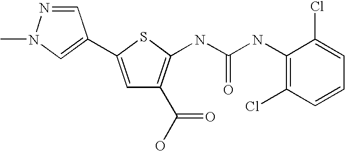 | 2-({[(2,6- dichlorophenyl)amino]carbonyl}amino)-5-(1-methyl- 1H-pyrazol-4-yl)-3- thiophenecarboxylic acid | 411 | 6.34 |
| 423 | 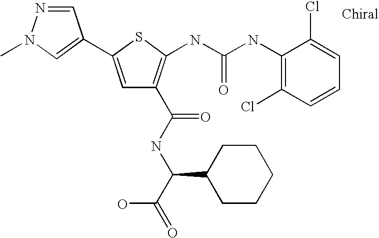 | (2S)-cyclohexyl({[2-({[(2,6- dichlorophenyl)amino]carbonyl}amino)-5-(1-methyl- 1H-pyrazol-4-yl)-3- thienyl]carbonyl}amino) ethanoic acid | 550 | 0.26 |
| 424 | 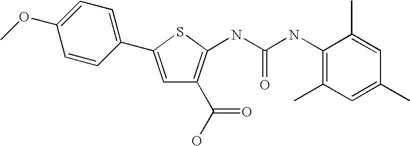 | 5-[4-(methyloxy)phenyl]-2- ({[(2,4,6- trimethylphenyl)amino]carbonyl}amino)-3- thiophenecarboxylic acid | 411 | 0.29 |
| 425 | 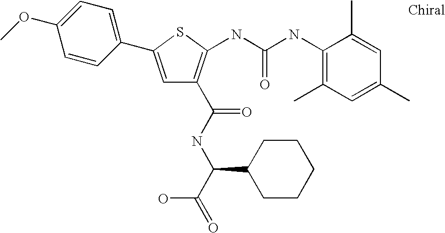 | (2S)-cyclohexyl({[5-[4- (methyloxy)phenyl]-2- ({[(2,4,6- trimethylphenyl)amino]carbonyl}amino)-3- thienyl]carbonyl}amino) ethanoic acid | 550 | 0.016 |
| 426 | 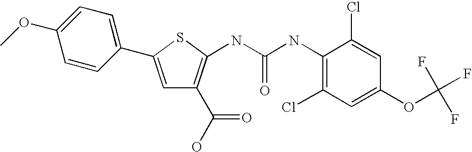 | 2-{[({2,6-dichloro-4- [(trifluoromethyl)oxy]phenyl}amino)carbonyl]amino}-5- [4-(methyloxy)phenyl]-3- thiophenecarboxylic acid | 521 | 1.31 |
| 427 | 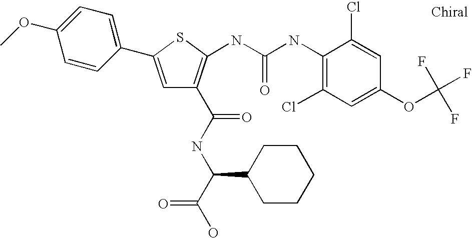 | (2S)-cyclohexyl[({2-{[({2,6- dichloro-4- [(trifluoromethyl)oxy]phenyl}amino)carbonyl]amino}-5- [4-(methyloxy)phenyl]-3- thienyl}carbonyl)amino]ethanoic acid | 660 | 0.027 |
| 428 | 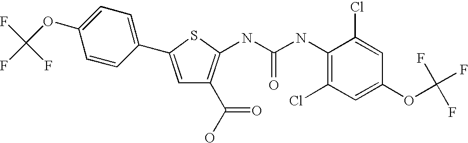 | 2-{[({2,6-dichloro-4- [(trifluoromethyl)oxy]phenyl}amino)carbonyl]mino}-5- {4- [(trifluoromethyl)oxy]phenyl}- 3-thiophenecarboxylic acid | 575 | 1.22 |
| 429 | 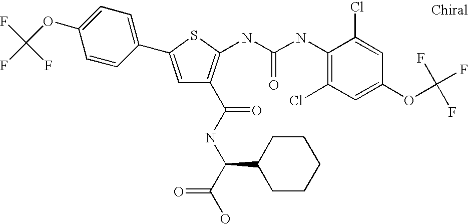 | (2S)-cyclohexyl{[(2-{[({2,6- dichloro-4- [(trifluoromethyl)oxy]phenyl}amino)carbonyl]amino}-5- {4- [(trifluoromethyl)oxy]phenyl}-3- thienyl)carbonyl]amino}ethanoic acid | 714 | 0.096 |
| 430 | 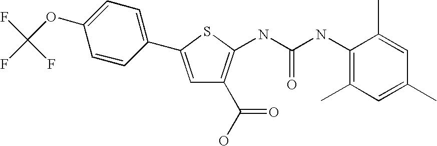 | 5-{4- [(trifluoromethyl)oxy]phenyl}- 2-({[(2,4,6- trimethylphenyl)amino]carbonyl}amino)-3- thiophenecarboxylic acid | 465 | 0.74 |
| 431 | 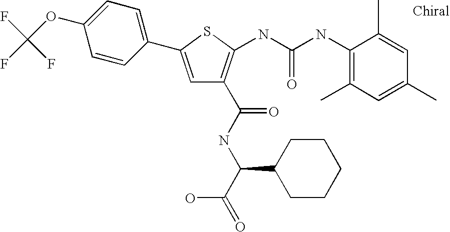 | (2S)-cyclohexyl({[5-{4- [(trifluoromethyl)oxy]phenyl}- 2-({[(2,4,6- trimethylphenyl)amino]carbonyl}amino)-3- thienyl]carbonyl}amino) ethanoic acid | 630 | 0.33 |
| 432 | 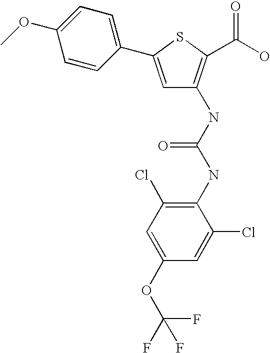 | 3-{[({2,6-dichloro-4- [(trifluoromethyl)oxy]phenyl}amino)carbonyl]amino}-5- [4-(methyloxy)phenyl]-2- thiophenecarboxylic acid | 521 | 2.7 |
| 433 | 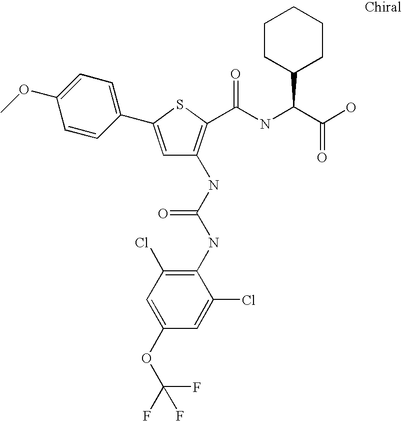 | (2S)-cyclohexyl[({3-{[({2,6- dichloro-4- [(trifluoromethyl)oxy]phenyl}amino)carbonyl]amino}-5- [4-(methyloxy)phenyl]-2- thienyl}carbonyl)amino]ethanoic acid | 660 | 0.08 |
| 434 | 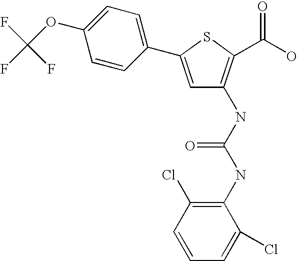 | 3-({[(2,6- dichlorophenyl)amino]carbonyl}amino)-5-{4- [(trifluoromethyl)oxy]phenyl}- 2-thiophenecarboxylic acid | 491 | 2.12 |
| 435 | 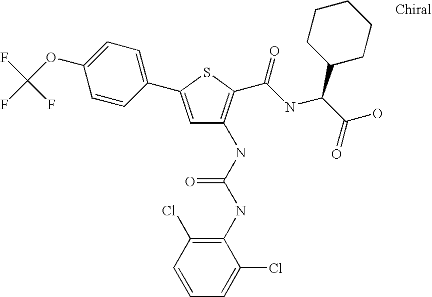 | (2S)-cyclohexyl{[(3-({[(2,6- dichlorophenyl)amino]carbonyl}amino)-5-{4- [(trifluoromethyl)oxy]phenyl}-2- thienyl)carbonyl]amino}ethanoic acid | 630 | 1.63 |
| 436 | 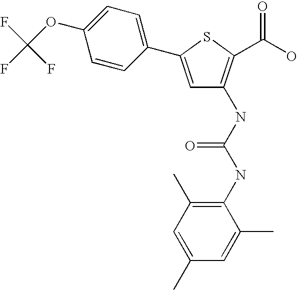 | 5-{4- [(trifluoromethyl)oxy]phenyl}-3- ({[(2,4,6- trimethylphenyl)amino]carbonyl}amino)-2- thiophenecarboxylic acid | 465 | 1.57 |
| 437 | 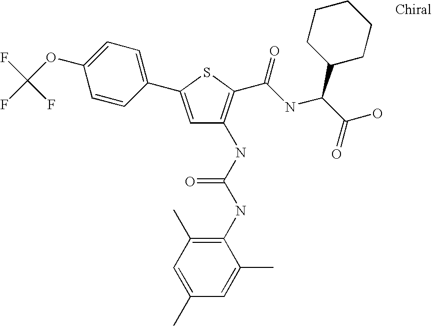 | (2S)-cyclohexyl({[5-{4- [(trifluoromethyl)oxy]phenyl}-3- ({[(2,4,6- trimethylphenyl)amino]carbonyl}amino)-2- thienyl]carbonyl}amino) ethanoic acid | 604 | 0.22 |
| 438 | 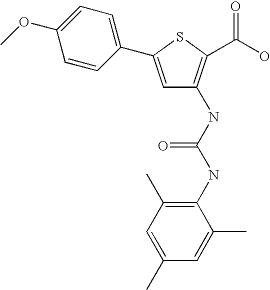 | 5-[4-(methyloxy)phenyl]-3- ({[(2,4,6- trimethylphenyl)amino]carbonyl}amino)-2- thiophenecarboxylic acid | 411 | 0.33 |
| 439 | 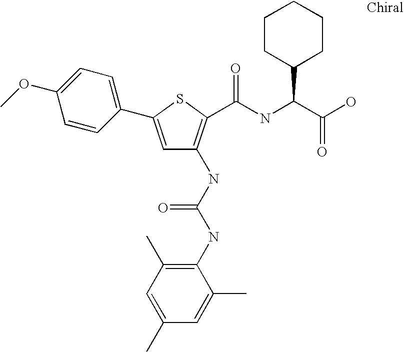 | (2S)-cyclohexyl({[5-[4- (methyloxy)phenyl]-3- ({[(2,4,6- trimethylphenyl)amino]carbonyl}amino)-2- thienyl]carbonyl}amino) ethanoic acid | 550 | 0.012 |
| 440 | 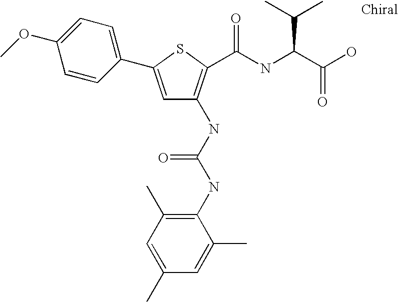 | N-{[5-[4- (methyloxy)phenyl]-3- ({[(2,4,6- trimethylphenyl)amino]carbonyl}amino)-2- thienyl]carbonyl}-L-valine | 510 | 0.1 |
| 441 | 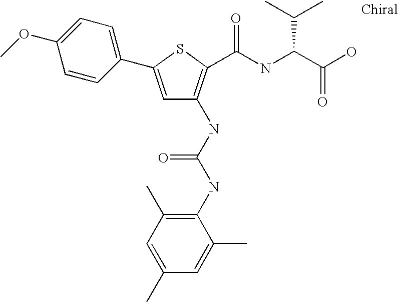 | N-{[5-[4- (methyloxy)phenyl]-3- ({[(2,4,6- trimethylphenyl)amino]carbonyl}amino)-2- thienyl]carbonyl}-D-valine | 510 | 2.11 |
| 442 | 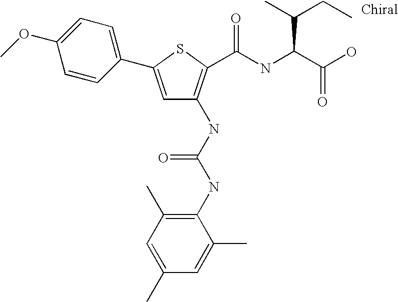 | N-{[5-[4- (methyloxy)phenyl]-3- ({[(2,4,6- trimethylphenyl)amino]carbonyl}amino)-2- thienyl]carbonyl}-L- isoleucine | 524 | 0.051 |
| 443 | 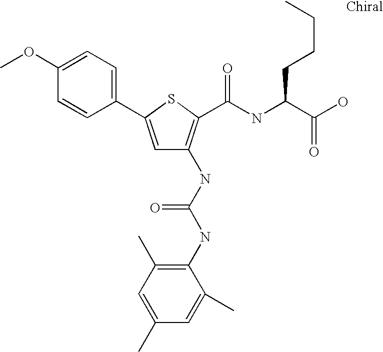 | N-{[5-[4- (methyloxy)phenyl]-3- ({[(2,4,6- trimethylphenyl)amino]carbonyl}amino)-2- thienyl]carbonyl}-L- norleucine | 524 | 0.043 |
| 444 | 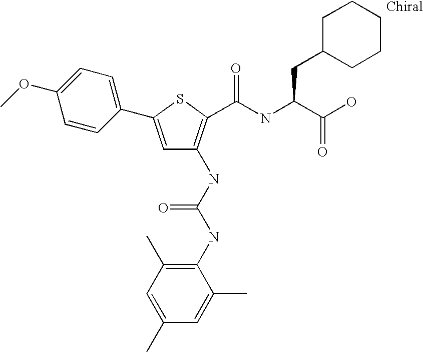 | 3-cyclohexyl-N-{[5-[4- (methyloxy)phenyl]-3- ({[(2,4,6- trimethylphenyl)amino]carbonyl}amino)-2- thienyl]carbonyl}-L-alanine | 564 | 0.11 |
| 445 | 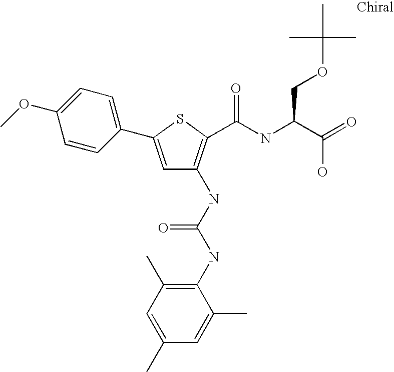 | O-(1,1-dimethylethyl)-N- {[5-[4-(methyloxy)phenyl]- 3-({[(2,4,6- trimethylphenyl)amino]carbonyl}amino)-2- thienyl]carbonyl}-L-serine | 554 | 0.082 |
| 446 | 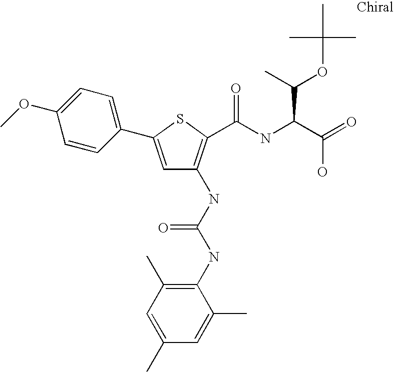 | O-(1,1-dimethylethyl)-N- {[5-[4-(methyloxy)phenyl]- 3-({[(2,4,6- trimethylphenyl)amino]carbonyl}amino)-2- thienyl]carbonyl}-L- threonine | 568 | 0.031 |
| 447 | 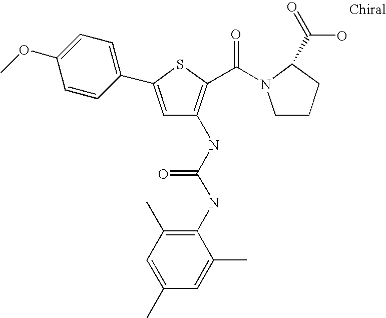 | 1-{[5-[4- (methyloxy)phenyl]-3- ({[(2,4,6- trimethylphenyl)amino]carbonyl}amino)-2- thienyl]carbonyl}-L-proline | 508 | 0.026 |
| 448 | 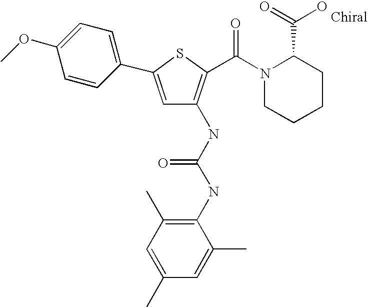 | (2S)-1-{[5-[4- (methyloxy)phenyl]-3- ({[(2,4,6- trimethylphenyl)amino]carbonyl}amino)-2- thienyl]carbonyl}-2- piperidinecarboxylic acid | 522 | 0.37 |
| 449 | 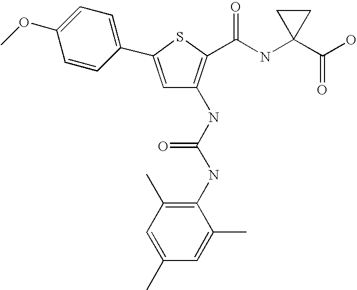 | 1-({[5-[4- (methyloxy)phenyl]-3- ({[(2,4,6- trimethylphenyl)amino]carbonyl}amino)-2- thienyl]carbonyl}amino) cyclopropanecarboxylic acid | 494 | 0.34 |
| 450 | 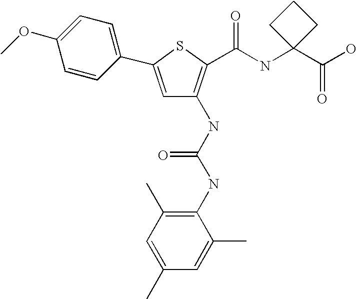 | 1-({[5-[4- (methyloxy)phenyl]-3- ({[(2,4,6- trimethylphenyl)amino]carbonyl}amino)-2- thienyl]carbonyl}amino) cyclobutanecarboxylic acid | 508 | 0.25 |
| 451 | 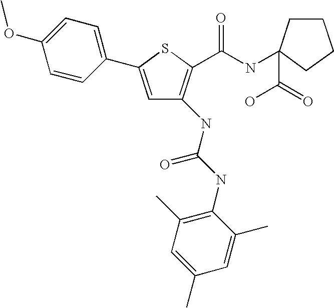 | 1-({[5-[4- (methyloxy)phenyl]-3- ({[(2,4,6- trimethylphenyl)amino]carbonyl}amino)-2- thienyl]carbonyl}amino) cyclopentanecarboxylic acid | 522 | 0.047 |
| 452 | 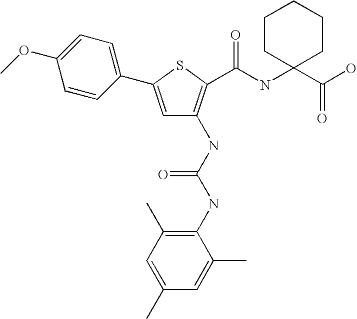 | 1-({[5-[4- (methyloxy)phenyl]-3- ({[(2,4,6- trimethylphenyl)amino]carbonyl}amino)-2- thienyl]carbonyl}amino) cyclohexanecarboxylic acid | 536 | 0.003 |
| 453 | 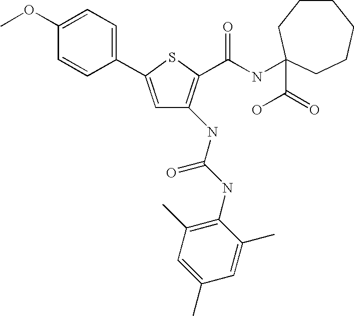 | 1-({[5-[4- (methyloxy)phenyl]-3- ({[(2,4,6- trimethylphenyl)amino]carbonyl}amino)-2- thienyl]carbonyl}amino) cycloheptanecarboxylic acid | 550 | 0.004 |
| 454 | 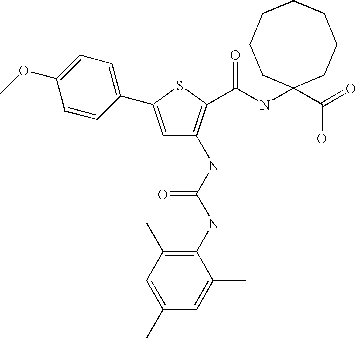 | 1-({[5-[4- (methyloxy)phenyl]-3- ({[(2,4,6- trimethylphenyl)amino]carbonyl}amino)-2- thienyl]carbonyl}amino) cycloctanecarboxylic acid | 564 | 0.001 |
| 455 | 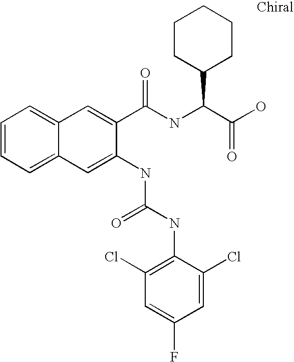 | (2S)-cyclohexyl({[3-({[(2,6- dichloro-4- fluorophenyl)amino]carbonyl}amino)-2- naphthalenyl]carbonyl}amino)ethanoic acid | 530 (M −H) | 0.086 |
| 456 | 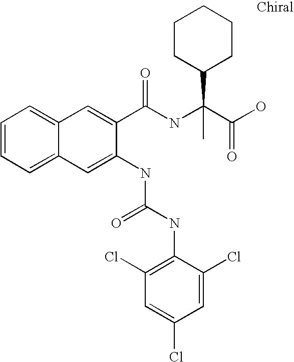 | 2-cyclohexyl-N-{[3- ({[(2,4,6- trichlorophenyl)amino]carbonyl}amino)-2- naphthalenyl]carbonyl}- L-alanine | 560 (M −H) | 0.23 |
| 457 | 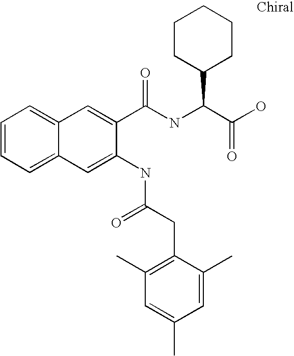 | (2S)-cyclohexyl{[(3- {[(2,4,6- trimethylphenyl)acetyl]amino}-2- naphthalenyl)carbonyl]amino}ethanoic acid | 485 (M −H) | 0.064 |
| 458 | 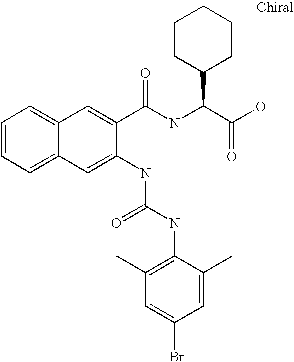 | (2S)-({[3-({[(4-bromo-2,6- dimethylphenyl)amino]carbonyl}amino)-2- naphthalenyl]carbonyl}amino)(cyclohexyl)ethanoic acid | 550 (M −H) | 0.22 |
| 459 | 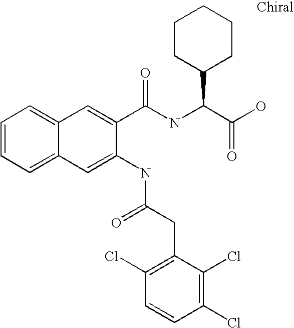 | (2S)-cyclohexyl{[(3- {[(2,3,6- trichlorophenyl)acetyl]amino}-2- naphthalenyl)carbonyl]amino}ethanoic acid | 545 (M −H) | 0.18 |
| 460 | 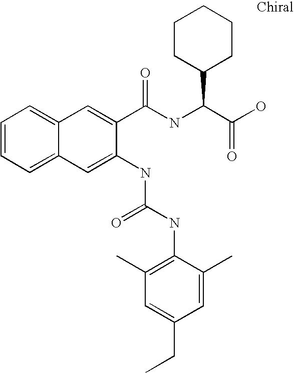 | (2S)-cyclohexyl({[3-({[(4- ethyl-2,6- dimethylphenyl)amino]carbonyl}amino)-2- naphthalenyl]carbonyl}amino)ethanoic acid | 500 (M −H) | 0.002 |
| 461 | 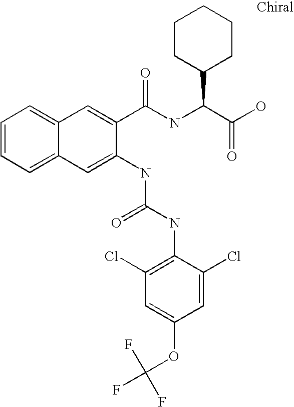 | (2S)-cyclohexyl{[(3-{[({2,6- dichloro-4- [(trifluoromethyl)oxy]phenyl}amino)carbonyl]amino}-2- naphthalenyl)carbonyl]amino}ethanoic acid | 596 (M −H) | 0.005 |
| 462 | 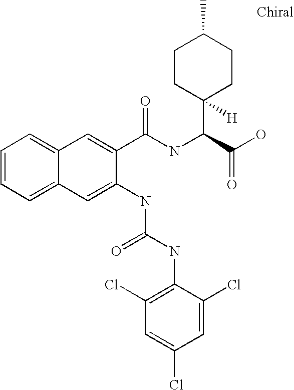 | (2S)-(trans-4- methylcyclohexyl)({[3- ({[(2,4,6- trichlorophenyl)amino]carbonyl}amino)-2- naphthalenyl]carbonyl}amino)ethanoic acid | 560 (M −H) | 0.056 |
| 463 | 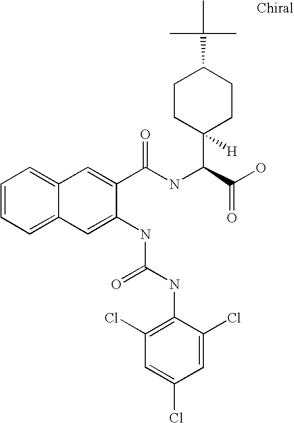 | (2S)-[trans-4-(1,1- dimethylethyl)cyclohexyl]({[3-({[(2,4,6- trichlorophenyl)amino]carbonyl}amino)-2- naphthalenyl]carbonyl}amino)ethanoic acid | 602 (M −H) | 0.56 |
| 464 | 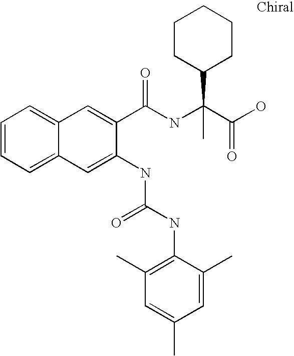 | 2-cyclohexyl-N-{[3- ({[(2,4,6- trimethylphenyl)amino]carbonyl}amino)-2- naphthalenyl]carbonyl}-L- alanine | 500 (M −H) | 0.008 |
| 465 | 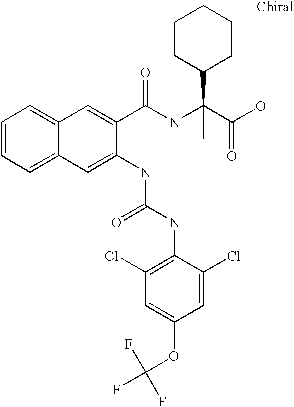 | 2-cyclohexyl-N-[(3-{[({2,6- dichloro-4- [(trifluoromethyl)oxy]phenyl}amino)carbonyl]amino}-2- naphthalenyl)carbonyl]-L- alanine | 610 (M −H) | 0.006 |
| 466 | 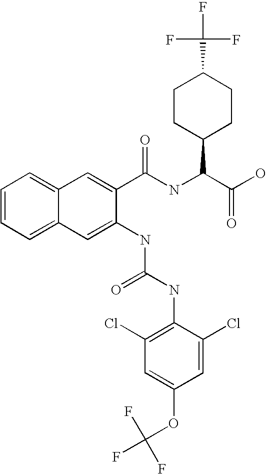 | {[(3-{[({2,6-dichloro-4- [(trifluoromethyl)oxy]phenyl}amino)carbonyl]amino}-2- naphthalenyl)carbonyl]amino}[trans-4- (trifluoromethyl)cyclohexyl]acetic acid | 664 (M −H) | 0.013 |
| 467 | 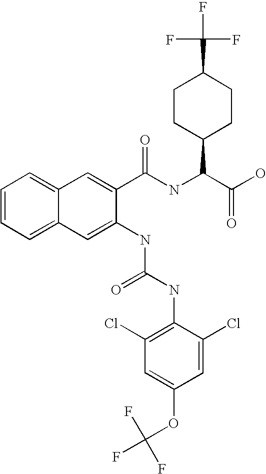 | {[(3-{[({2,6-dichloro-4- [(trifluoromethyl)oxy]phenyl}amino)carbonyl]amino}-2- naphthalenyl)carbonyl]amino}[cis-4- (trifluoromethyl)cyclohexyl]acetic acid | 664 (M −H) | 0.035 |
| 468 | 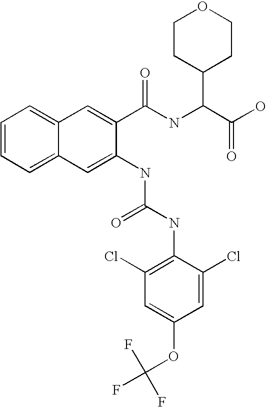 | {[(3-{[({2,6-dichloro-4- [(trifluoromethyl)oxy]phenyl}amino)carbonyl]amino}-2- naphthalenyl)carbonyl]amino}(tetrahydro-2H-pyran-4- yl)acetic acid | 558 (M −H) | 0.015 |
| 469 | 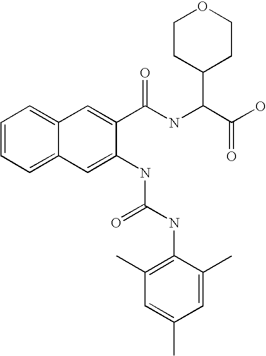 | tetrahydro-2H-pyran-4- yl({[3-({[(2,4,6- trimethylphenyl)amino]carbonyl}amino)-2- naphthalenyl]carbonyl}amino)acetic acid | 488 (M −H) | 0.059 |
| 470 | 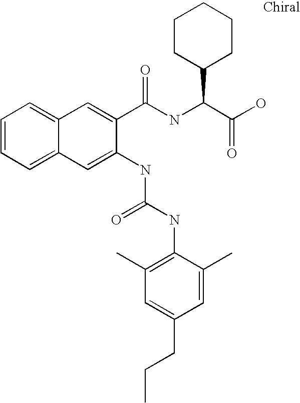 | (2S)-cyclohexyl({[3-({[(2,6- dimethyl-4- propylphenyl)amino]carbonyl}amino)-2- naphthalenyl]carbonyl}amino)ethanoic acid | 514 (M −H) | 0.002 |
| 471 | 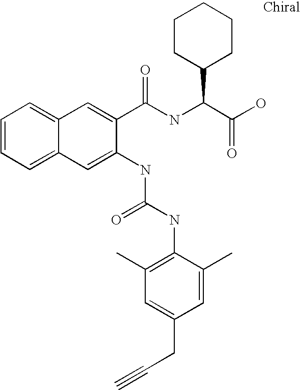 | (2S)-cyclohexyl[({3-[({[2,6- dimethyl-4-(2-propyn-1- yl)phenyl]amino}carbonyl) amino]-2- naphthalenyl}carbonyl) amino]ethanoic acid | 510 (M −H) | 0.003 |
| 472 | 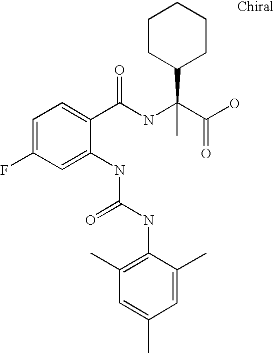 | 2-cyclohexyl-N-{[4-fluoro-2- ({[(2,4,6- trimethylphenyl)amino]carbonyl}amino)phenyl]carbonyl}- L-alanine | 468 (M −H) | 0.16 |
| 473 | 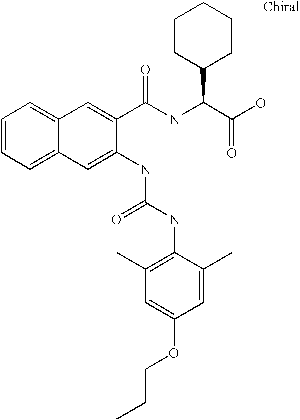 | (2S)-cyclohexyl[({3-[({[2,6- dimethyl-4- (propyloxy)phenyl]amino}carbonyl)amino]-2- naphthalenyl}carbonyl) amino]ethanoic acid | 530 (M −H) | 0.001 |
| 474 | 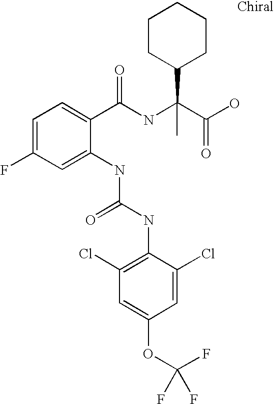 | 2-cyclohexyl-N-[(2-{[({2,6- dichloro-4- [(trifluoromethyl)oxy]phenyl}amino)carbonyl]amino}-4- fluorophenyl)carbonyl]-L- alanine | 578 (M −H) | 0.145 |
| 475 | 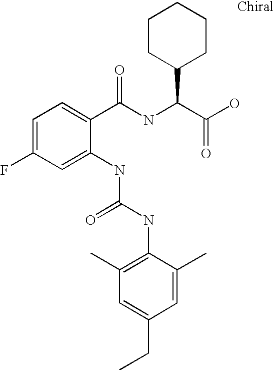 | (2S)-cyclohexyl({[2-({[(4- ethyl-2,6- dimethylphenyl)amino]carbonyl}amino)-4- fluorophenyl]carbonyl}amino)ethanoic acid | 468 (M −H) | 0.051 |
| 476 | 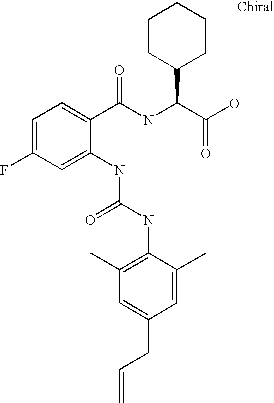 | (2S)-cyclohexyl[({2-[({[2,6- dimethyl-4-(2-propen-1- yl)phenyl]amino}carbonyl) amino]-4- fluorophenyl}carbonyl) amino]ethanoic acid | 480 (M −H) | 0.03 |
| 477 | 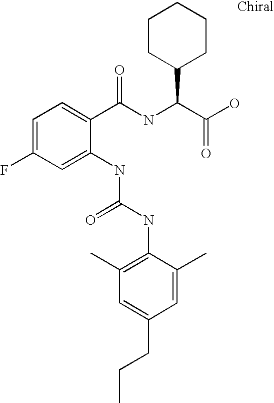 | (2S)-cyclohexyl({[2-({[(2,6- dimethyl-4- propylphenyl)amino]carbonyl}amino)-4- fluorophenyl]carbonyl}amino)ethanoic acid | 482 (M −H) | 0.008 |
| 478 | 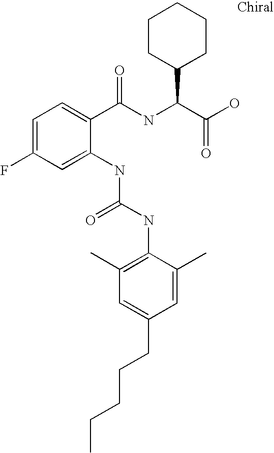 | (2S)-cyclohexyl({[2-({[(2,6- dimethyl-4- pentylphenyl)amino]carbonyl}amino)-4- fluorophenyl]carbonyl}amino)ethanoic acid | 510 (M −H) | 0.003 |
| 479 | 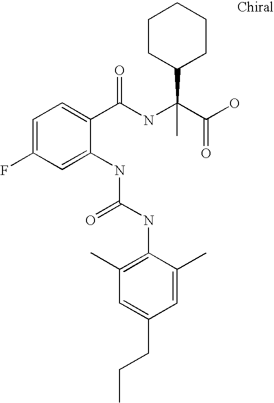 | 2-cyclohexyl-N-{[2-({[(2,6- dimethyl-4- propylphenyl)amino]carbonyl}amino)-4- fluorophenyl]carbonyl}-L- alanine | 496 (M −H) | 0.004 |
| 480 | 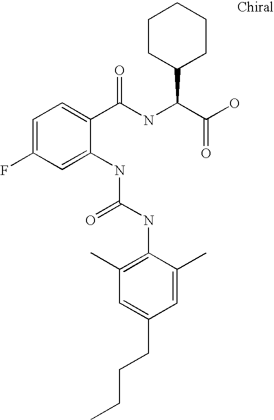 | (2S)-({[2-({[(4-butyl-2,6- dimethylphenyl)amino]carbonyl}amino)-4- fluorophenyl]carbonyl}amino)(cyclohexyl)ethanoic acid | 496 (M −H) | 0.02 |
| 481 | 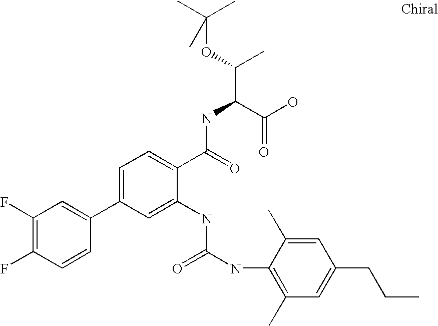 | O-(1,1-dimethylethyl)-N- {[3-({[(2,6-dimethyl-4- propylphenyl)amino]carbonyl}amino)-3′,4′-difluoro-4- biphenylyl]carbonyl}-L- threonine | 594 (M −H) | 0.0004 |
| 482 | 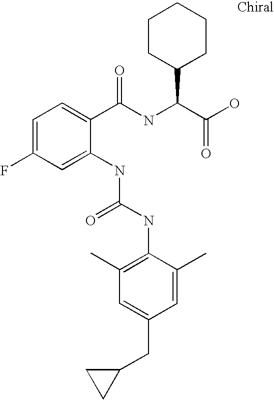 | (2S)-cyclohexyl[({2-[({[4- (cyclopropylmethyl)-2,6- dimethylphenyl]amino}carbonyl)amino]-4- fluorophenyl}carbonyl) amino]ethanoic acid | 494 (M −H) | 0.005 |
| 483 | 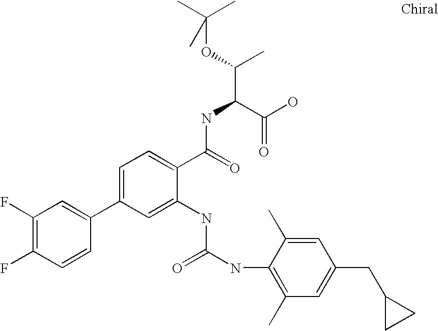 | N-({3-[({[4- (cyclopropylmethyl)-2,6- dimethylphenyl]amino}carbonyl)amino]-3′,4′-difluoro-4- biphenylyl}carbonyl)-O- (1,1-dimethylethyl)-L- threonine | 606 (M −H) | 0.001 |
| 484 | 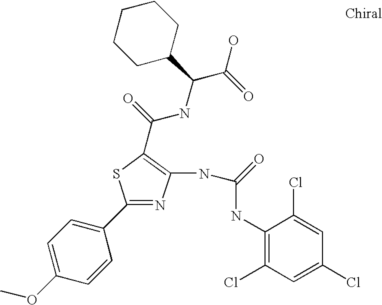 | (2S)-cyclohexyl({[2-[4- (methyloxy)phenyl]-4- ({[(2,4,6- trichlorophenyl)amino]carbonyl}amino)-1,3-thiazol-5- yl]carbonyl}amino)ethanoic acid | 611 (M +H) | 5 |
| 485 | 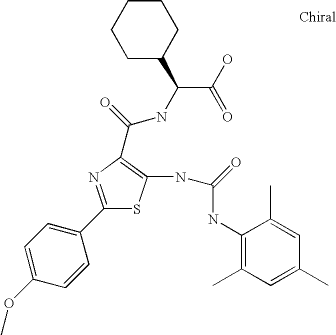 | (2S)-cyclohexyl({[2-[4- (methyloxy)phenyl]-5- ({[(2,4,6- trimethylphenyl)amino]carbonyl}amino)-1,3-thiazol-4- yl]carbonyl}amino)ethanoic acid | 551 (M +H), 573 (M +Na) | 0.7 |
| 486 | 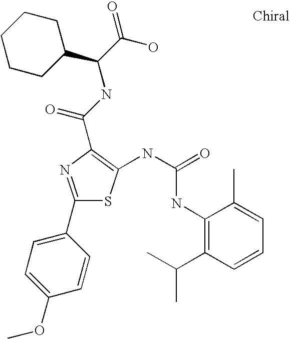 | (2S)-cyclohexyl[({5-[({[2- methyl-6-(1- methylethyl)phenyl]amino}carbonyl)amino]-2-[4- (methyloxy)phenyl]-1,3- thiazol-4- yl}carbonyl)amino]ethanoic acid | 565 (M +H), 587 (M +Na) | 1.78 |
| 487 | 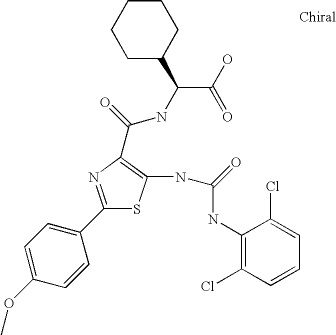 | (2S)-cyclohexyl[({5-({[(2,6- dichlorophenyl)amino]carbonyl}amino)-2-[4- (methyloxy)phenyl]-1,3- thiazol-4- yl}carbonyl)amino]ethanoic acid | 577 (M +H), 599 (M +Na) | 0.74 |
| 488 | 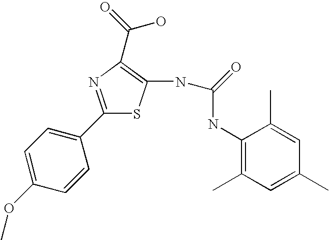 | 2-[4-(Methyloxy)phenyl]-5- ({[(2,4,6- trimethylphenyl)amino]carbonyl}amino)-1,3-thiazole-4- carboxylic acid | | 8.23 |
| 489 | 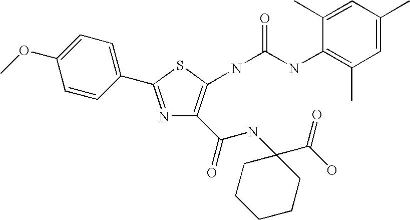 | 1-({[2-[4- (Methyloxy)phenyl]-5- ({[(2,4,6- trimethylphenyl)amino]carbonyl}amino)-1,3-thiazol-4- yl]carbonyl}amino) cyclohexanecarboxylic acid | 537 (M +H), 559 (M +Na) | 0.034 |
| 490 | 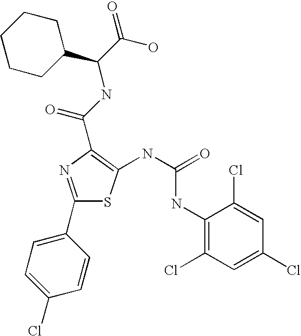 | (2S)-({[2-(4-chlorophenyl)- 5-({[(2,4,6- trichlorophenyl)amino]carbonyl}amino)-1,3-thiazol-4- yl]carbonyl}amino) (cyclohexyl)ethanoic acid | 615 (M +H) | 0.17 |
| 491 | 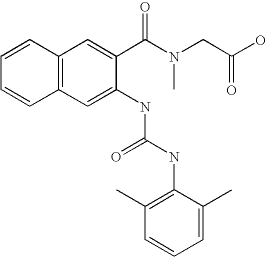 | N-{[3-({[(2,6- dimethylphenyl)amino]carbonyl}amino)-2- naphthalenyl]carbonyl}-N- methylglycine | NMR in example | 2.11 |
| 492 | 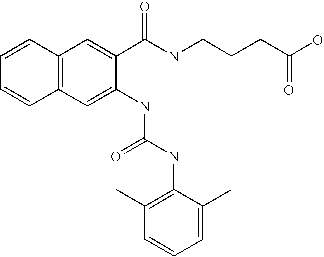 | 4-({[3-({[(2,6- dimethylphenyl)amino]carbonyl}amino)-2- naphthalenyl]carbonyl}amino)butanoic acid | NMR in example | 7.52 |
| 493 | 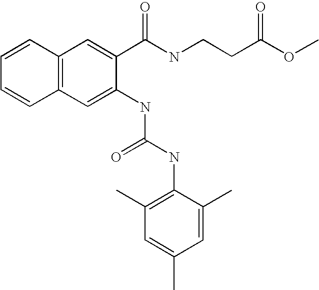 | methyl N-{[3-({[(2,4,6- trimethylphenyl)amino]carbonyl}amino)-2- naphthalenyl]carbonyl}- beta-alaninate | NMR in example | 9.86 |
| 494 | 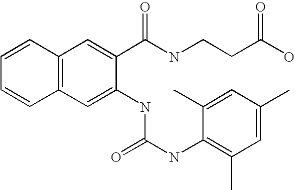 | N-{[3-({[(2,4,6- trimethylphenyl)amino]carbonyl}amino)-2- naphthalenyl]carbonyl}- beta-alanine | NMR in example | 0.36 |
| 495 | 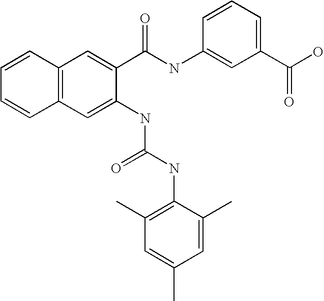 | 3-({[3-({[(2,4,6- trimethylphenyl)amino]carbonyl}amino)-2- naphthalenyl]carbonyl}amino)benzoic acid | NMR in example | 0.3 |
| 496 | 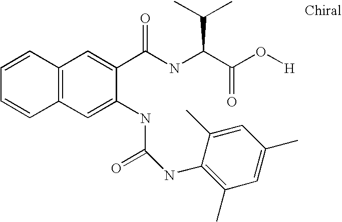 | N-{[3-({[(2,4,6- trimethylphenyl)amino]carbonyl}amino)-2- naphthalenyl]carbonyl}-L- valine | NMR in example | 0.29 |
| 497 | 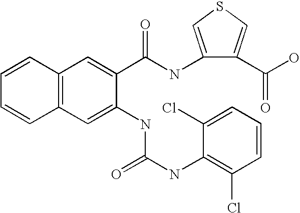 | 4-({[3-({[(2,6- dichlorophenyl)amino]carbonyl}amino)-2- naphthalenyl]carbonyl}amino)-3-thiophenecarboxylic acid | NMR in example | 17.66 |
| 498 | 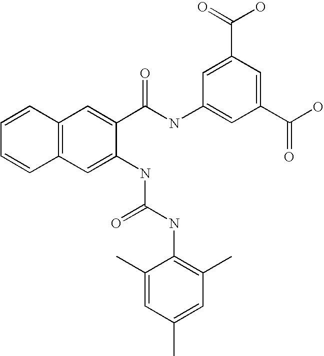 | 5-({[3-({[(2,4,6- trimethylphenyl)amino]carbonyl}amino)-2- naphthalenyl]carbonyl}amino)-1,3- benzenedicarboxylic acid | 512 (M +H) | 0.11 |
| 499 | 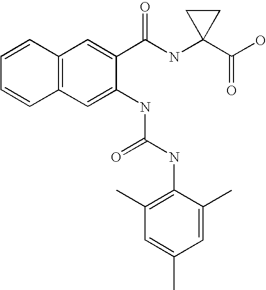 | 1-({[3-({[(2,4,6- trimethylphenyl)amino]carbonyl}amino)-2- naphthalenyl]carbonyl}amino)cyclopropanecarboxylic acid | NMR in example | 0.51 |
| 500 | 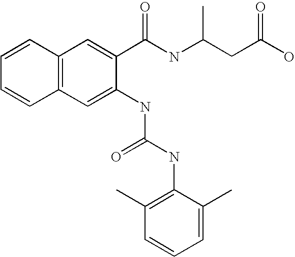 | 3-({[3-({[(2,6- dimethylphenyl)amino]carbonyl}amino)-2- naphthalenyl]carbonyl}amino)butanoic acid | NMR in example | 2.14 |
| 501 | 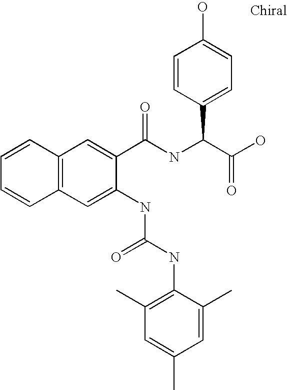 | (2S)-(4-hydroxyphenyl)({[3- ({[(2,4,6- trimethylphenyl)amino]carbonyl}amino)-2- naphthalenyl]carbonyl}amino)ethanoic acid | 496 (M −H) | 0.012 |
| 502 | 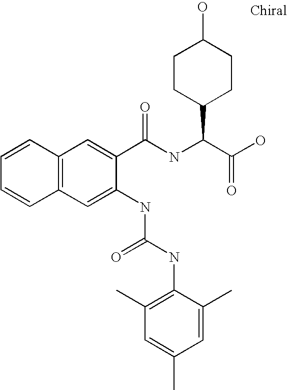 | (2S)-(4- hydroxycyclohexyl)({[3- ({[(2,4,6- trimethylphenyl)amino]carbonyl}amino)-2- naphthalenyl]carbonyl}amino)ethanoic acid | 502 (M −H) | 0.013 |
| 503 | 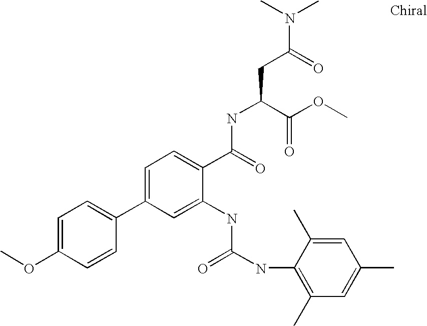 | Methyl N4,N4-dimethyl- N2-{[4′-(methyloxy)-3- ({[(2,4,6- trimethylphenyl)amino]carbonyl}amino)-4- biphenylyl]carbonyl}-L- asparaginate | ACPI 561 (M +H) | 11.1 |
| 504 | 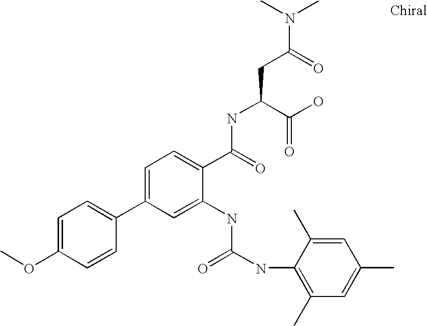 | N⁴,N⁴-dimethyl-N²-{[4′- (methyloxy)-3-({[(2,4,6- trimethylphenyl)amino]carbonyl}amino)-4- biphenylyl]carbonyl}-L- asparagine | APCI 547 (M +H) | 0.032 |
| 505 | 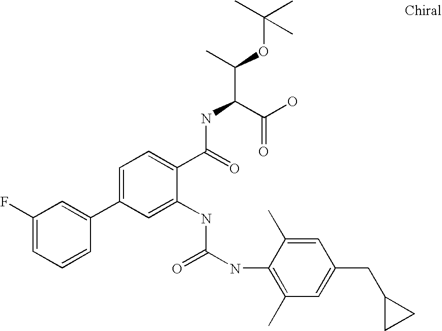 | N-({3-[({[4- (cyclopropylmethyl)-2,6- dimethylphenyl]amino}carbonyl)amino]-3′-fluoro-4- biphenylyl}carbonyl)-O- (1,1-dimethylethyl)-L- threonine | APCI 588 (M −H) | 0.003 |
| 506 | 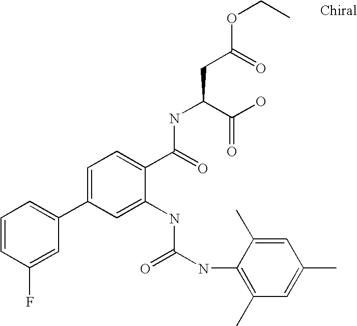 | (2S)-4-(ethyloxy)-2-({[3′- fluoro-3-({[(2,4,6- trimethylphenyl)amino]carbonyl}amino)-4- biphenylyl]carbonyl}amino)- 4-oxobutanoic acid | APCI 536 (M +H) | 1.09 |
| 507 | 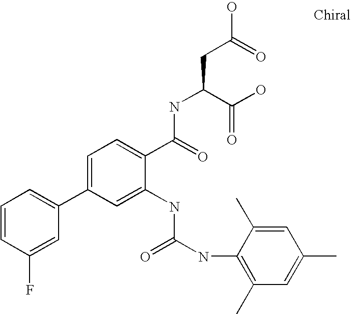 | N-{[3′-fluoro-3-({[(2,4,6- trimethylphenyl)amino]carbonyl}amino)-4- biphenylyl]carbonyl}-L- aspartic acid | APCI 508 (M +H) | 0.053 |
| 508 | 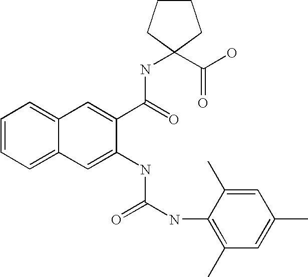 | 1-({[3-({[(2,4,6- trimethylphenyl)amino]carbonyl}amino)-2- naphthalenyl]carbonyl}amino)cyclopentanecarboxylic acid | 460 (M +H) | 0.279 |
| 509 | 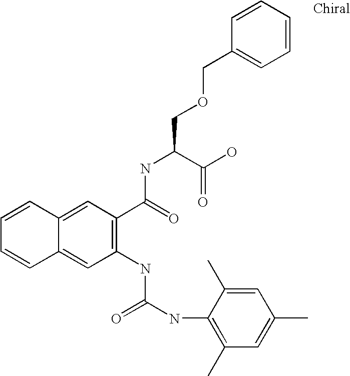 | O-(Phenylmethyl)-N-{[3- ({[(2,4,6- trimethylphenyl)amino]carbonyl}amino)-2- naphthalenyl]carbonyl}-L- serine | 526 (M +H) | 0.028 |
| 510 | 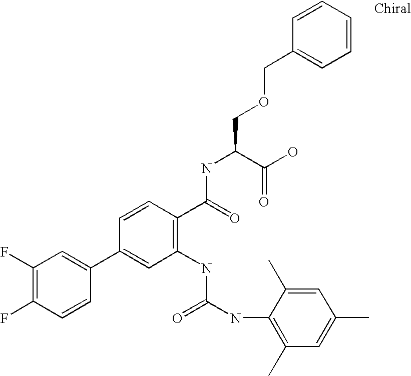 | N-{[3′,4′-Difluoro-3- ({[(2,4,6- trimethylphenyl)amino]carbonyl}amino)-4- biphenylyl]carbonyl}-O- (phenylmethyl)-L-serine | 588 (M +H) | 0.042 |
| 511 | 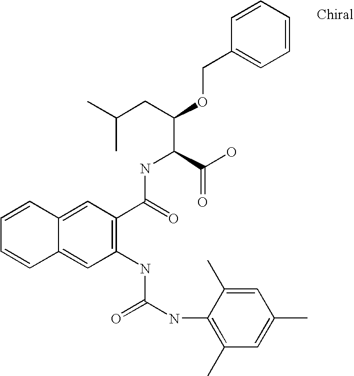 | (3R)-5-Methyl-3- [(phenylmethyl)oxy]-N-{[3- ({[(2,4,6- trimethylphenyl)amino]carbonyl}amino)-2- naphthalenyl]carbonyl}-L- norleucine | 582 (M +H) | 0.077 |
| 512 | 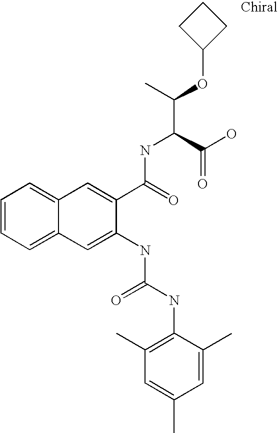 | O-cyclobutyl-N-{[3- ({[(2,4,6- trimethylphenyl)amino]carbonyl}amino)-2- naphthalenyl]carbonyl}-L- threonine | 504 (M +H) | 0.011 |
| 513 | 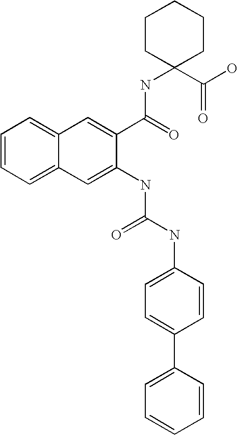 | 1-{[(3-{[(4- biphenylylamino)carbonyl]amino}-2- naphthalenyl)carbonyl]amino}cyclohexanecarboxylic acid | 508 (M +H) | 7.43 |
| 514 | 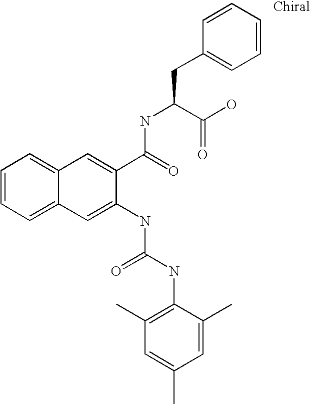 | N-{[3-({[(2,4,6- trimethylphenyl)amino]carbonyl}amino)-2- naphthalenyl]carbonyl}-L- phenylalanine | 496 (M +H) | 0.052 |
| 515 | 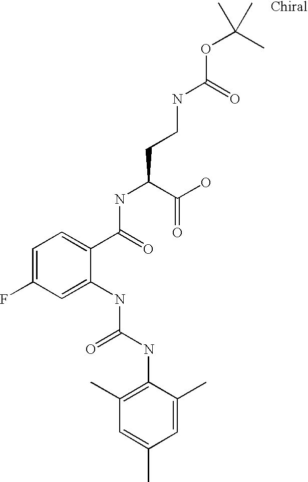 | (2S)-4-({[(1,1- dimethylethyl)oxy]carbonyl}amino)-2-({[4-fluoro-2- ({[(2,4,6- trimethylphenyl)amino]carbonyl}amino)phenyl]carbonyl}amino)butanoic acid | 517 (M +H) | 1.9 |
| 516 | 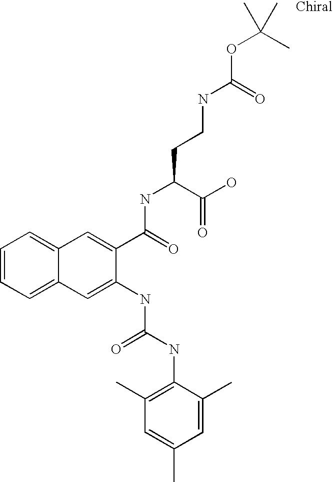 | (2S)-4-({[(1,1- dimethylethyl)oxy]carbonyl}amino)-2-({[3-({[(2,4,6- trimethylphenyl)amino]carbonyl}amino)-2- naphthalenyl]carbonyl}amino)butanoic acid | 549 (M +H) | 0.089 |
| 517 | 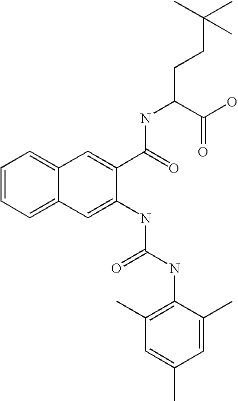 | 5,5-dimethyl-N-{[3- ({[(2,4,6- trimethylphenyl)amino]carbonyl}amino)-2- naphthalenyl]carbonyl}norleucine | 489 (M +H) | 0.019 |
| 518 | 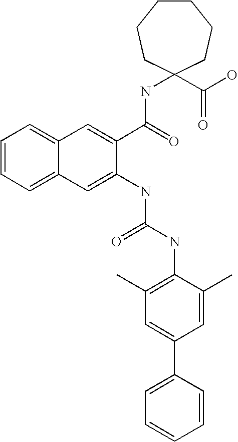 | 1-({[3-({[(3,5-dimethyl-4- biphenylyl)amino]carbonyl}amino)-2- naphthalenyl]carbonyl}amino)cycloheptanecarboxylic acid | 550 (M +H) | 0.34 |
| 519 | 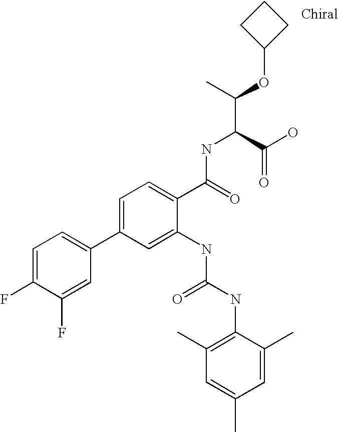 | O-cyclobutyl-N-{[3′,4′- difluoro-3-({[(2,4,6- trimethylphenyl)amino]carbonyl}amino)-4- biphenylyl]carbonyl}-L- threonine | 566 (M +H) | 0.007 |
| 520 | 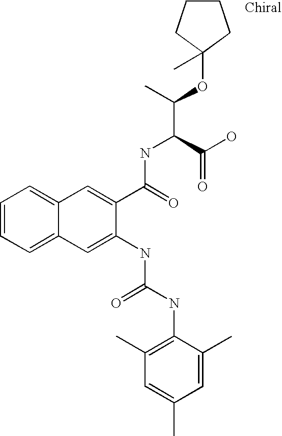 | O-(1-methylcyclopentyl)-N- {[3-({[(2,4,6- trimethylphenyl)amino]carbonyl}amino)-2- naphthalenyl]carbonyl}-L- threonine | 566 (M +H) | 0.006 |
| 521 | 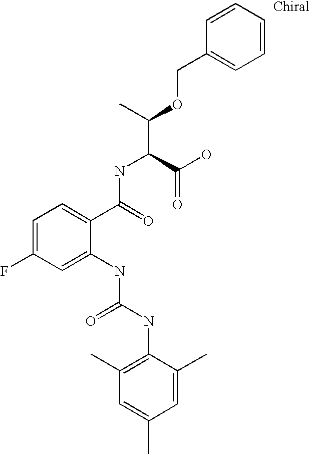 | N-{[4-fluoro-2-({[(2,4,6- trimethylphenyl)amino]carbonyl}amino)phenyl]carbonyl}-O-(phenylmethyl)-L- threonine | 532 (M +H) | 0.134 |
| 522 | 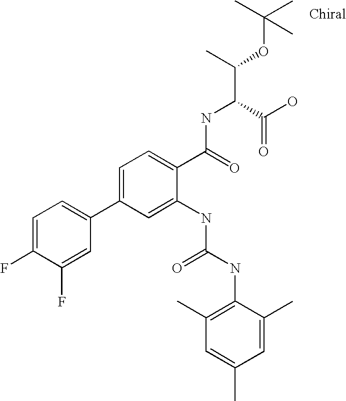 | N-{[3′,4′-difluoro-3-({[(2,4,6- trimethylphenyl)amino]carbonyl}amino)-4- biphenylyl]carbonyl}-O- (1,1-dimethylethyl)-D- threonine | 568 (M +H) | 0.675 |
| 523 | 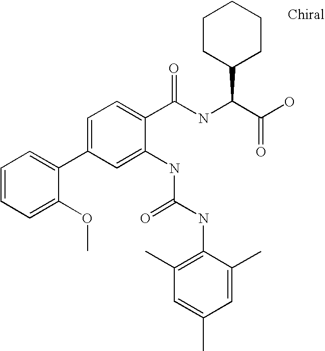 | (2S)-cyclohexyl({[2′- (methyloxy)-3-({[(2,4,6- trimethylphenyl)amino]carbonyl}amino)-4- biphenylyl]carbonyl}amino) ethanoic acid | 544 (M +H) | 0.008 |
| 524 | 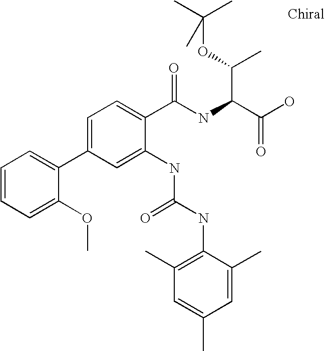 | O-(1,1-Dimethylethyl)-N- {[2′-(methyloxy)-3-({[(2,4,6- trimethylphenyl)amino]carbonyl}amino)-4- biphenylyl]carbonyl}-L- threonine | 562 (M +H) | 0.011 |
| 525 | 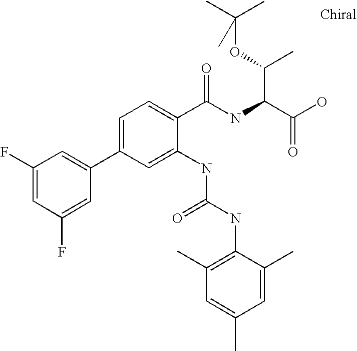 | N-{[3′,5′-Difluoro-3- ({[(2,4,6- trimethylphenyl)amino]carbonyl}amino)-4- biphenylyl]carbonyl}-O- (1,1-dimethylethyl)-L- threonine | 566 (M −H) | 0.007 |
| 526 | 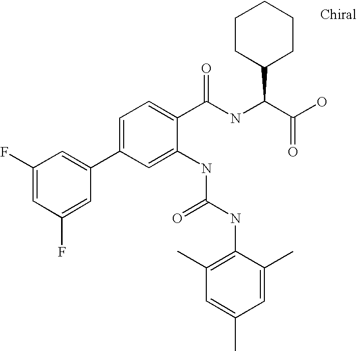 | (2S)-Cyclohexyl({[3′,5′- difluoro-3-({[(2,4,6- trimethylphenyl)amino]carbonyl}amino)-4- biphenylyl]carbonyl}amino) ethanoic acid | 550 (M +H) (APCI) | 0.005 |
| 527 | 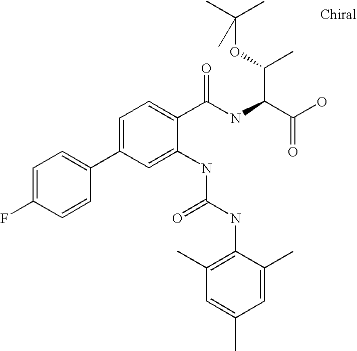 | O-(1,1-Dimethylethyl)-N- {[4′-fluoro-3-({[(2,4,6- trimethylphenyl)amino]carbonyl}amino)-4- biphenylyl]carbonyl}-L- threonine | 550 (M +H) (APCI) | 0.003 |
| 528 | 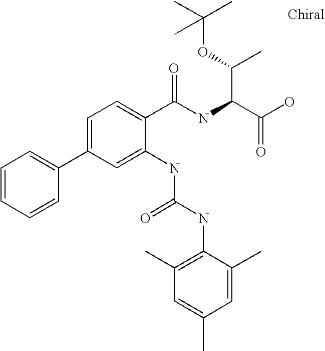 | O-(1,1-Dimethylethyl)-N- {[3-({[(2,4,6- trimethylphenyl)amino]carbonyl}amino)-4- biphenylyl]carbonyl}-L- threonine | 532 (M +H) (APCI) | 0.003 |
| 529 | 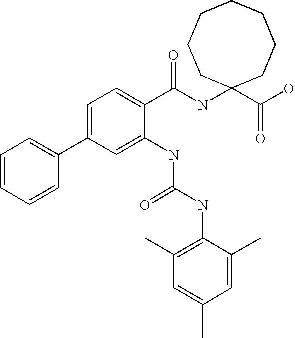 | 1-({[3-({[(2,4,6- Trimethylphenyl)amino]carbonyl}amino)-4- biphenylyl]carbonyl}amino) cyclooctanecarboxylic acid | 528 (M +H) (APCI | 0.002 |
| 530 | 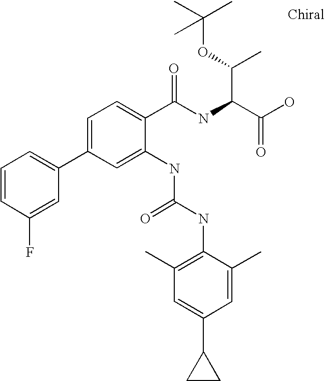 | N-{[3-({[(4-Cyclopropyl-2,6- dimethylphenyl)amino]carbonyl}amino)-3′-fluoro-4- biphenylyl]carbonyl}-O- (1,1-dimethylethyl)-L- threonine | 576 (M +H) (APCI | 0.021 |
| 531 | 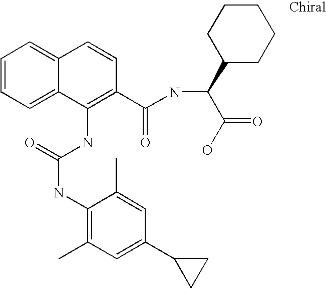 | (2S)-cyclohexyl({[3-({[(4- cyclopropylphenyl)amino]carbonyl}amino)-2- naphthalenyl]carbonyl}amino)ethanoic acid | 514 (M +H) | 0.019 |
| 532 | 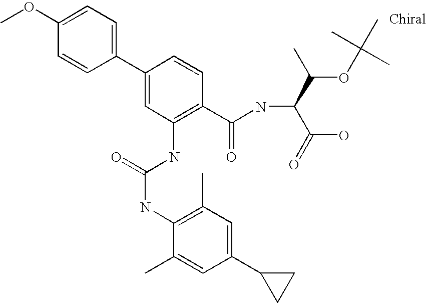 | N-{[3-({[(4-cyclopropyl-2,6- dimethylphenyl)amino]carbonyl}amino)-4′- (methyloxy)-4- biphenylyl]carbonyl}-O- (1,1-dimethylethyl)-L- threonine | 588 (M +H) | 0.011 |
| 533 | 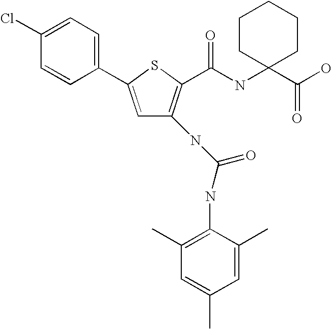 | 1-({[5-(4-chlorophenyl)-3- ({[(2,4,6- trimethylphenyl)amino]carbonyl}amino)-2- thienyl]carbonyl}amino) cyclohexanecarboxylic acid | 540 (M +H) | 0.003 |
| 534 | 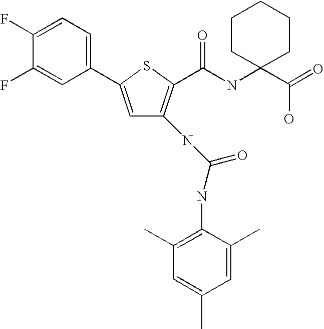 | 1-({[5-(3,4-difluorophenyl)- 3-({[(2,4,6- trimethylphenyl)amino]carbonyl}amino)-2- thienyl]carbonyl}amino) cyclohexanecarboxylic acid | 542 (M +H) | 0.104 |
| 535 | 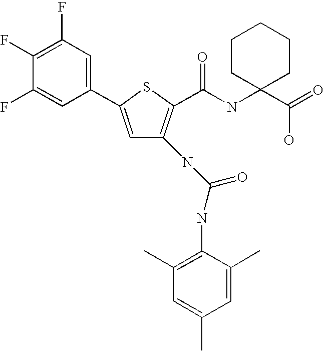 | 1-({[5-(3,4,5- trifluorophenyl)-3-({[(2,4,6- trimethylphenyl)amino]carbonyl}amino)-2- thienyl]carbonyl}amino) cyclohexanecarboxylic acid | 560 (M +H) | 0.206 |

What is claimed is:

1. A compound of Formula 1 comprising: 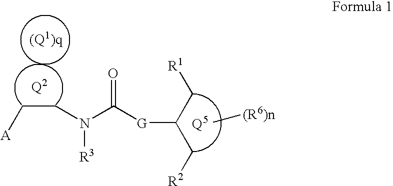
a pharmaceutically acceptable salt, solvate, or physiologically functional derivative thereof
wherein:
A is C(═O)NQ³Q⁴ or C(═O)OH;
Q¹ and Q² are fused together;
Q¹ is selected from the group consisting of (i) a 5- or 6-membered aromatic ring, (ii) a 5- or 6-membered cycloalkyl ring, (iii) a 5- or 6-membered heteroaromatic ring having at least one heteroatom selected from the group consisting of nitrogen, oxygen, or sulfur, and (iv) a 4- to 8-membered heterocyclic ring having at least one heteroatom selected from the group consisting of nitrogen, oxygen, or sulfur; and q is 0 or 1;
Q² is selected from the group consisting of (i) a 5- or 6-membered aromatic ring and (ii) a 5- or 6-membered heteroaromatic ring having at least one heteroatom selected from the group consisting of nitrogen, oxygen, or sulfur;
R¹ and R² are each independently selected from the group consisting of hydrogen, C₁₋₆ alkyl, halo, alkoxy, monoalkylamino, and dialkylamino;
R³ is hydrogen or a C₁₋₆ alkyl;
Q³ and Q⁴ are each independently selected from the group consisting of (i) hydrogen, (ii) C₁₋₆ alkyl, (iii) —CR⁴R⁵Z, where Z is a 5- or 6-membered heteroaryl having at least one heteroatom selected from the group consisting of nitrogen, oxygen, and sulfur, (iv) aryl, and (v) —CR⁴R⁵COOH;
R⁴ and R⁵ are each independently selected from the group consisting of (i) hydrogen, (ii) a C₁₋₆ alkyl, (iii) a 4- to 8-membered cycloalkyl, (iv) a 5- or 6-membered aryl, (v) a 5- or 6-membered heteroaryl, (vi) a 5- or 6-membered aralkyl, (vii) a 5- or 6-membered heteroaralkyl, having at least one heteroatom selected from the group consisting of nitrogen, oxygen and sulfur, (viii) a 4- to 8-membered cycloalkylalkyl, and (ix) a 4- to 8-membered heterocyclic ring;
R⁴ and R⁵ are taken together can form a (i) 3-10 membered cycloalkyl or (ii) a 4-8 membered heterocyclic ring;
G is selected from the group consisting of carbon, nitrogen, oxygen, and sulfur;
Q⁵ is selected from the group consisting of (i) a 5- or 6-membered aromatic ring and (ii) a 5- or 6-membered heteroaromatic ring having at least one heteroatom selected from the group consisting of nitrogen, oxygen, and sulfur;
R⁶ is selected from the group consisting of (i) C₁₋₆ alkyl, (ii) halogen, (iii) alkoxy, (iv) cyano, (v) hydroxyl, (vi) haloalkyl, (vii) a mono- or dialkyl- amino, (viii) 3-5 membered cycloalkyl, (ix) 3-5 membered cycloalkylalkyl, (x) alkenyl, (xi) alkynyl, and (xii) acyl; and n is 0 or 1.

2. The compound of claim 1 wherein q is 1.

3. The compound of claim 1 wherein Q¹ is cyclohexyl or phenyl.

4. The compound of claim 3 wherein Q¹ is phenyl.

5. The compound of claim 1 wherein Q² is substituted.

6. The compound of claim 5 wherein Q² is substituted with an alkoxy or a halo.

7. The compound of claim 1 wherein Q² is selected from the group consisting of an unsubstituted aromatic ring, a dimethoxy substituted aromatic ring, and a mono- or dihalosubstituted aromatic ring.

8. The compound of claim 1 wherein Q² is an unsubstituted phenyl.

9. The compounds of claim 1 wherein q is 0.

10. The compound of claim 9 wherein Q² is substituted phenyl, substituted thienyl, or substituted pyridyl ring.

11. The compound of claim 10 wherein Q² is substituted with mono- or di-halo, mono- or di-alkyl, or mono or di-alkoxy.

12. The compound of claim 10 wherein Q² is substituted with an aryl ring.

13. The compound of claim 12 wherein said aryl is a phenyl ring.

14. The compound of claim 13 wherein said phenyl ring is substituted.

15. The compound of claim 14 wherein said phenyl is substituted with a halo or alkoxy group.

16. The compound of claim 1 wherein R¹ and R² are each independently selected from the group consisting of halo and C₁₋₆ alkyl.

17. The compound of claim 1 wherein R¹ is chloro and R² is methyl or vice versa.

18. The compound of claim 1 wherein R¹ and R² are each chloro.

19. The compound of claim 1 wherein R¹ and R² are each methyl.

20. The compound of claim 1 wherein R³ is hydrogen.

21. The compound of claim 1 wherein Q³ and Q⁴ are each independently selected from the group consisting of (i) —CR⁴R⁵Z where Z is tetrazole, (ii) —CR⁴R⁵COOH, and (iii) hydrogen.

22. The compound of claim 1 wherein Q³ is —CR⁴R⁵COOH and Q⁴ is hydrogen.

23. The compound of claim 1 wherein R⁴ and R⁵ are selected from the group consisting of (i) hydrogen, (ii), cycloalkyl, (iii) aryl, (iv) substituted or unsubstituted C₁₋₆ alkyl, and (v) aralkyl.

24. The compound of claim 21 wherein R⁴ and R⁵ are selected from the group consisting of hydrogen, aryl, cycloalkyl, and substituted and unsubstituted C₁₋₆ alkyl.

25. The compound of claim 24 wherein said substituted C₁₋₆ alkyl is substituted with alkoxy or —COOH.

26. The compound of claim 1 wherein R⁴ and R⁵ taken together form a (i) 3-10 membered cycloalkyl or (ii) a 4-8 membered heterocyclic ring.

27. The compound of claim 1 wherein G is carbon or nitrogen.

28. The compound of claim 1 wherein Q⁵ is a substituted or unsubstituted 6-membered aromatic ring.

29. The compound of claim 28 wherein Q⁵ is phenyl, alkylphenyl, or halophenyl.

30. The compound of claim 1 wherein Q² is phenyl and q is 1.

31. The compound of claim 1 wherein R⁶ is C₁₋₅ alkyl, halomethyl, alkoxy, or halo.

32. The compound of claim 1 wherein R⁶ is methyl, ethyl, n-propyl, cyclopropylmethyl, chloro, or trifluoromethoxy.

33. The compound of claim 1 wherein Q² is a substituted or unsubstituted heteroaromatic ring.

34. The compound of claim 26 wherein Q² is a 5-membered heteroaromatic ring with one sulfur as the heteroatom.

35. The compound of claim 1 wherein said compound is selected from the group consisting of
N-[3-({[(2,6-dimethylphenyl)amino]carbonyl}amino)-2-naphthoyl]glycine;
Phenyl({[3-({[(2,4,6-trimethylphenyl)amino]carbonyl}amino)-2-naphthalenyl]carbonyl}amino)acetic acid;
(2S)-Cyclohexyl({[3-({[(2,6-dichlorophenyl)amino]carbonyl}amino)-2-naphthalenyl]carbonyl}amino)acetic acid;
(2S)({[4-chloro-2-({[(2,6-dichlorophenyl)amino]carbonyl}amino)phenyl]carbonyl}amino)(cyclohexyl) ethanoic acid;
(2S)-Cyclohexyl{[3-({[(2,4,6-trichlorophenyl)amino]carbonyl}amino)-2-naphthoyl]amino}ethanoic acid;
(2S)-Cyclohexyl{[3-({[(2-ethyl-6-methylphenyl)amino]carbonyl}amino)-2-naphthoyl]amino}ethanoic acid;
(2S)-({3-[({[2-Chloro-6-(trifluoromethyl)phenyl]amino}carbonyl)amino]-2-naphthoyl}amino)(cyclohexyl) ethanoic acid;
(2S)-Cyclohexyl[(3-{[(2,4,6-trichlorophenyl)acetyl]amino}-2-naphthoyl)amino]ethanoic acid;
(2S)-Cyclohexyl[(3-{[(mesitylamino)carbonyl]amino}-2-naphthoyl) amino]ethanoic acid;
(2S)-Cyclohexyl({[4,5-dichloro-2-({[(2,6-dichlorophenyl)amino]carbonyl}amino)phenyl]carbonyl}amino)ethanoic acid;
(2S)-({[4-Chloro-2-({[(2,4,6-trimethylphenyl)amino]carbonyl}aminophenyl]carbonyl}amino)(cyclohexyl)ethanoic acid;
(2S)-Cyclohexyl({[4,5-dichloro-2-({[(2,6-dimethylphenyl)amino]carbonyl}amino)phenyl]carbonyl}amino)ethanoic acid;
(2S)-Cyclohexyl({[2-({[(2,6-dimethylphenyl)amino]carbonyl}amino)-4-(3-pyridinyl)phenyl]carbonyl}amino)ethanoic acid;
(2S)-Cyclohexyl({[3-({[(2,4,6-trimethylphenyl)amino]carbonyl}amino)-4-biphenylyl]carbonyl}amino)ethanoic acid;
(2S)-Cyclohexyl({[2-({[(2,6-dimethylphenyl)amino]carbonyl}amino)-4-(2-thienyl)phenyl]carbonyl}amino)ethanoic acid;
(2S)-Cyclohexyl({[3-({[(2,6-dimethylphenyl)amino]carbonyl}amino)-4′-hydroxy-4-biphenylyl]carbonyl}amino)ethanoic acid;
(2S)-Cyclohexyl({[3-({[(2,6-dimethylphenyl)amino]carbonyl}amino)-3′ 74′-difluoro-4-biphenylyl]carbonyl}amino)ethanoic acid;
(2S)-Cyclohexyl({[3-({[(2,6-dimethylphenyl)amino]carbonyl}amino)-4′-(methyloxy)-4-biphenylyl]carbonyl}amino)ethanoic acid;
(2S)-Cyclohexyl({[4′-(methyloxy)-3-({[(2,4,6-trimethylphenyl)amino]carbonyl}amino)-4-biphenylyl]carbonyl}amino)ethanoic acid;
(2S)-Cyclohexyl({[4′-hydroxy-3-({[(2,4,6-trimethylphenyl)amino]carbonyl}amino)-4-biphenylyl]carbonyl}amino)ethanoic acid;
(2S)-Cyclohexyl({[4′-nitro-3-({[(2,4,6-trimethylphenyl)amino]carbonyl}amino)-4-biphenylyl]carbonyl}amino)ethanoic acid;
(2S)-Cyclohexyl({[4′-(hydroxymethyl)-3-({[(2,4,6-trimethylphenyl)amino]carbonyl}amino)-4-biphenyly]carbonyl}amino)ethanoic acid;
(2S)-({[4′-Amino-3-({[(2,4,6-trimethylphenyl)amino]carbonyl}amino)-4-biphenylyl]carbonyl}amino)(cyclohexyl)ethanoic acid;
(2S)-Cyclohexyl({[3-({[(2,6-dichlorophenyl)amino]carbonyl}amino)-4-biphenylyl]carbonyl}amino)ethanoic acid;
(2S)-Cyclohexyl({[4-{[(methylamino)carbonyl]amino}-2-({[(2,4,6-trimethylphenyl)amino]carbonyl}amino)phenyl]carbonyl}amino)ethanoic acid;
(2S)-Cyclohexyl({[3′,4′-difluoro-3-({[(2,4,6-trimethylphenyl)amino]carbonyl}amino)-4-biphenylyl]carbonyl}amino)ethanoic acid;
(2S)-Cyclopentyl({[4′-(methyloxy)-3-({[(2,4,6-trimethylphenyl)amino]carbonyl}amino)-4-biphenylyl]carbonyl}amino)ethanoic acid;
(2S)-Cyclohexyl{[(3-{[({2,6-dichloro-4-[(trifluoromethyl)oxy]phenyl}amino)carbonyl]amino}-3′,4′-difluoro-4-biphenylyl)carbonyl]amino}ethanoic acid;
(2S)-Cyclohexyl({[4′-[(dimethylamino)methyl]-3-({[(2,4,6-trimethylphenyl)amino]carbonyl}amino)-4-biphenylyl]carbonyl}amino)ethanoic acid;
(2S)-Cyclohexyl{[(3-{[({2,6-dichloro-4-[(trifluoromethyl)oxy]phenyl}amino)carbonyl]amino}-4-biphenylyl)carbonyl]amino}ethanoic acid;
(2S)-Cyclohexyl({[3-{[({2,6-dichloro-4-[(trifluoromethyl)oxy]phenyl}amino)carbonyl]amino}4′-(methyloxy)-4-biphenylyl]carbonyl}amino)ethanoic acid;
(2S)-Cyclohexyl({[4′-(1-pyrrolidinylmethyl)-3-({[(2,4,6-trimethyphenyl)amino]carbonyl}amino)-4-biphenylyl]carbonyl}amino)ethanoic acid;
(2S)-cyclohexyl({[4′-(4-morpholinylmethyl)-3-({[(2,4,6-trimethylphenyl)amino]carbonyl}amino)-4-biphenylyl]carbonyl}amino)ethanoic acid;
(2S)-Cyclohexyl({[4′-(ethyloxy)-3-({[(2,4,6-trimethylphenyl)amino]carbonyl}amino)-4-biphenylyl]carbonyl}amino)ethanoic acid;
N-{[4′-(methyloxy)-3-({[(2,4,6-trimethylphenyl)amino]carbonyl}amino)-4-biphenylyl]carbonyl}-L-norleucine;
1-({[4′-(methyloxy)-3-({[(2,4,6-trimethylphenyl)amino]carbonyl}amino)-4-biphenylyl]carbonyl}amino)cycloheptanecarboxylic acid;
(2S)-Cyclohexyl({[4′-fluoro-3-({[(2,4,6-trimethylphenyl)amino]carbonyl}amino)-4-biphenylyl]carbonyl}amino)ethanoic acid;
(2S)-({[4-(1,3-Benzodioxol-5-yl)-2-({[(2,4,6-trimethylphenyl)amino]carbonyl}amino)phenyl]carbonyl}amino)(cyclohexyl)ethanoic acid;
O-(1,1-Dimethylethyl)-N-{[4′-(methyloxy)-3-({[(2,4,6-trimethylphenyl)amino]carbonyl}amino)-4-biphenylyl]carbonyl}-L-threonine;
1-({[3′4′-Difluoro-3-({[(2,4,6-trimethylphenyl)amino]carbonyl}amino)-4-biphenylyl]carbonyl}amino)cyclooctanecarboxylic acid;
(2S)-Cyclohexyl({[4-(2,3-dihydro-1,4-benzodioxin-6-yl)-2-({[(2,4,6-trimethylphenyl)amino]carbonyl}amino)phenyl]carbonyl}amino)ethanoic acid;
(2S)-({[3′,4′-Bis(methyloxy)-3-({[(2,4,6-trimethylphenyl)amino]carbonyl}amino)-4-biphenylyl]carbonyl}amino)(cyclohexyl)ethanoic acid;
(2S)-Cyclohexyl({[4,5-difluoro-2-({[(2,4,6-trimethylphenyl)amino]carbonyl}amino)phenyl]carbonyl}amino)ethanoic acid;
1-({[4′-(Methyloxy)-3-({[(2,4,6-trimethylphenyl)amino]carbonyl}amino)-4-biphenylyl]carbonyl}amino)cyclooctanecarboxylic acid;
N-{[3-{[((2,6-Dichloro-4-[(trifluoromethyl)oxy]phenyl}amino)carbonyl]amino)-4′-(methyloxy)-4-biphenylyl]carbonyl}-O-(1,1-dimethylethyl)-L-threonine;
O-(1,1-Dimethylethyl)-N-{[3′-fluoro-3-({[(2,4,6-trimethylphenyl)amino]carbonyl}amino)-4-biphenylyl]carbonyl}-L-threonine;
(2S)-Cyclohexyl({[3′-fluoro-3-({[(2,4,6-trimethylphenyl)amino]carbonyl}amino)-4-biphenylyl]carbonyl}amino)ethanoic acid;
O-(1,1-Dimethylethyl)-N-{[3′-fluoro-4′-(methyloxy)-3-({[(2,4,6-trimethylphenyl)amino)carbonyl}amino)-4-biphenylyncarbonyl}-threonine;
O-(1,1-Dimethylethyl)-N-{[3-({[(2,6-dimethyl-4-propylphenyl)amino]carbonyl}amino)-4′-(methyloxy)-4-biphenylyl]carbonyl}-L-threonine;
(2S)-Cyclohexyl({[3′-fluoro-4′-(methyloxy)-3-({[(2,4,6-trimethylphenyl)amino]carbonyl}amino)-4-biphenylyl]carbonyl}amino)ethanoic acid;
1-({[3′-Fluoro-4′-(methyloxy)-3-({[(2,4,6-trimethylphenyl)amino]carbonyl}amino)-4-biphenylyl]carbonyl}amino)cyclooctanecarboxylic acid;
N-{[3-{[({2,4,6-trimethylphenyl)amino]carbonyl}amino)-2-naphthalenyl]carbonyl}-L-norleucine;
O-(1,1-dimethylethyl)-N-{[3-({[(2,4,6-trimethylphenyl)amino]carbonyl}amino)-2-naphthalenyl]carbonyl}-L-serine;
5-methyl-N-{[3-({[(2,4,6-trimethylphenyl)amino]carbonyl}amino)-2-naphthalenyl]carbonyl}norleucine;
6,6,6-trifluoro-N-{[3-({[(2,4,6-trimethylphenyl)amino]carbonyl}amino)-2-naphthalenyl]carbonyl}norleucine;
O-(1,1-dimethylethyl)-N-{[3-({[(2,4,6-trimethylphenyl)amino]carbonyl}amino)-2-naphthalenyl]carbonyl}-L-threonine;
N-{[3-({[(2,4,6-trimethylphenyl)amino]carbonyl}amino)-2-naphthalenyl]carbonyl}-L-leucine;
N-{[3-({[(2,4,6-trimethylphenyl)amino]carbonyl}amino)-2-naphthalenyl]carbonyl}-L-isoleucine;
N-{[3-({[(2,4,6-trimethylphenyl)amino]carbonyl}amino)-2-naphthalenyl]carbonyl}-L-norvaline;
O-(1,1-dimethylethyl)-N-[(3-{[(2,4,6-trimethylphenyl)acetyl]amino}-2-naphthalenyl)carbonyl]-L-threonine;
O-butyl-N-{[3-({[(2,4,6-trimethylphenyl)amino]carbonyl}amino)-2-naphthalenyl]carbonyl}-L-serine;
O-[2-(methyloxy)ethyl]-N-{[3-({[(2,4,6-trimethylphenyl)amino]carbonyl}amino)-2-naphthalenyl]carbonyl}-L-serine;
O-ethyl-N-{[3-({[(2,4,6-trimethylphenyl)amino]carbonyl}amino)-2-naphthalenyl]carbonyl}-L-serine;
O-(1-methylethyl)-N-{[3-({[(2,4,6-trimethylphenyl)amino]carbonyl}-amino)-2-naphthalenyl]carbonyl}-L-serine;
O-(2,2-dimethylpropyl)-N-{[3-({[(2,4,6-trimethylphenyl)amino]carbonyl}amino)-2-naphthalenyl]carbonyl}-L-serine;
O-(tetrahydro-2H-pyran-4-yl)-N-{[3-({[(2,4,6-trimethylphenyl)amino]carbonyl}amino)-2-naphthalenyl]carbonyl}-L-serine;
O-(1-methylethyl)-N-{[3-({[(2,4,6-trimethylphenyl)amino]carbonyl}amino)-2-naphthalenyl]carbonyl}-L-threonine;
(2S)-Cyclohexyl({[3-({[(2,4,6-trimethylphenyl)amino]carbonyl}amino)-2-quinolinyl]carbonyl}amino)ethanoic acid;
1-({[3-({[(2,4,6-trimethylphenyl)amino]carbonyl}amino)-2-naphthalenyl]carbonyl}amino)cycloheptanecarboxylic acid;
1-({[3-({[(2,4,6-trichlorophenyl)amino]carbonyl}amino)-2-naphthalenyl]carbonyl}amino)cyclooctanecarboxylic acid;
1-({[3-({[(2,4,6-trimethylphenyl)amino]carbonyl}amino)-2-naphthalenyl]carbonyl}amino)cyclooctanecarboxylic acid;
1-({[3-({[(2,4,6-trimethylphenyl)amino]carbonyl}amino)-2-naphthalenyl]carbonyl}amino)cyclodecanecarboxylic acid;
1-({[3-({[(2,4,6-trimethylphenyl)amino]carbonyl}amino)-2-quinolinyl]carbonyl}amino)cycloheptanecarboxylic acid;
1-({[3-({[(2,4,6-trimethylphenyl)amino]carbonyl}amino)-2-quinolinyl]carbonyl}amino)cyclooctanecarboxylic acid;
1-({[3-({[(2,6-dimethyl-4-propylphenyl)amino]carbonyl}amino)-2-naphthalenyl]carbonyl}amino)cycloheptanecarboxylic acid;
2-({[3-({[(2,4,6-trimethylphenyl)amino]carbonyl}amino)-2-naphthalenyl]carbonyl}amino)-2,3-dihydro-1H-indene-2-carboxylic acid;
2-({[3-({[(2,4,6-trimethylphenyl)amino]carbonyl}amino)-2-naphthalenyl]carbonyl}amino)-1,2,3,4-tetrahydro-2-naphthalenecarboxylic acid;
1-({[5-Chloro-3-({[(2,6-dimethyl-4-propylphenyl)amino]carbonyl}amino)-2-pyridinyl]carbonyl}amino)cyclooctanecarboxylic acid,
(2S)-Cyclohexyl({[5-[4-(methyloxy)phenyl]-3-({[(2,4,6-trimethylphenyl)amino]carbonyl}amino)-2-pyridinyl]carbonyl}amino)ethanoic acid;
1-({[5-[4-(methyloxy)phenyl]-3-({[(2,4,6-trimethylphenyl)amino]carbonyl}amino)-2-pyridinyl]carbonyl}amino)cycloheptanecarboxylic acid;
O-(phenylmethyl)-N-{[3-({[(2,4,6-trimethylphenyl)amino]carbonyl}amino)-2-naphthalenyl]carbonyl}-L-threonine;
(3R)-3-[(phenylmethyl)oxy]-N-{[3-({[(2,4,6-trimethylphenyl)amino]carbonyl}amino)-2-naphthalenyl]carbonyl}-L-norvaline;
(2S)-(4,4-difluorocyclohexyl)({[3-({[(2,4,6-trimethylphenyl)amino]carbonyl}amino)-2-naphthalenyl]carbonyl}amino)ethanoic acid;
(2S)-cyclopentyl({[3-({[(2,4,6-trimethylphenyl)amino]carbonyl}amino)-2-naphthalenyl]carbonyl}amino)ethanoic acid;
1,4-dioxaspiro[4.5]dec-8-yl({[3-({[(2,4,6-trimethylphenyl)amino]carbonyl}amino)-2-naphthalenyl]carbonyl}amino)acetic acid;
(cis and trans)-[4-({[(1,1-dimethylethyl)oxy]carbonyl}amino)cyclohexyl]({[3-({[(2,4,6-trimethylphenyl)amino]carbonyl}amino)-2-naphthalenyl]carbonyl}amino)acetic acid;
(cis and trans)-(4-{[(methylamino)carbonyl]amino}cyclohexyl)({[3-({[(2,4,6-trimethylphenyl)amino]carbonyl}amino)-2-naphthalenyl]carbonyl}amino)acetic acid;
N-{[3-({[(2,4,6-trimethylphenyl)amino]carbonyl}amino)-2-naphthalenyl]carbonyl}-L-aspartic acid;
N-[(3-{[({2,6-dichloro-4-[(trifluoromethyl)oxy]phenyl}amino)carbonyl]amino)-2-naphthalenyl)carbonyl]-L-aspartic acid;
N-{[3-({[(2,4,6-trimethylphenyl)amino]carbonyl}amino)-2-naphthalenyl]carbonyl}-D-aspartic acid;
(2S)-[(1S)-3-oxocyclohexyl]({[3-({[(2,4,6-trimethylphenyl)amino]carbonyl}am ino)-2-naphthalenyl]carbonyl}amino)ethanoic acid;
(2S)-[(1S)-3-hydroxycyclohexyl]({[3-({[(2,4,6-trimethylphenyl)amino]carbonyl}amino)-2-naphthalenyl]carbonyl}amino)ethanoic acid;
(2S)-{(1S)-3-[(trifluoroacetyl)oxy]cyclohexyl}({[3-({[(2,4,6-trimethylphenyl)amino]carbonyl}amino)-2-naphthalenyl]carbonyl}amino)ethanoic acid;
N-{[4′-(methyloxy)-3-({[(2,4,6-trimethylphenyl)amino]carbonyl}amino)-4-biphenylyl]carbonyl}-L-aspartic acid;
(2S)-2-({[3-({[(2,6-dimethyl-4-propylphenyl)amino]carbonyl}amino)-4′-(methyloxy)-4-biphenylyl]carbonyl}amino)-4-(ethyloxy)-4-oxobutanoic acid;
N-{[3-({[(2,6-dimethyl-4-propylphenyl)amino]carbonyl}amino)-4′-(methyloxy)-4-biphenylyl]carbonyl}-L-aspartic acid;
N-{[3′,4′-difluoro-3-({[(2,4,6-trimethylphenyl)amino]carbonyl}amino)-4-biphenylyl]carbonyl}-O-(1,1-dimethylethyl)-L-threonine;
N-{[3′,4′-difluoro-3-({[(2,4,6-trimethylphenyl)amino]carbonyl}amino)-4-biphenylyl]carbonyl}-L-aspartic acid;
N²-{[4′-(methyloxy)-3-({[(2,4,6-trimethylphenyl)amino]carbonyl}amino)-4-biphenylyl]carbonyl}-L-asparagine;
N-{[3′,4′-difluoro-3-({[(2,4,6-trimethylphenyl)amino]carbonyl}amino)-4-biphenylyl]carbonyl}-L-glutamic acid;
(2S)-cyclohexyl[({3-({[(2,6-dichlorophenyl)amino]carbonyl}amino)-5-[4-(methyloxy)phenyl]-2-thienyl}carbonyl)amino]ethanoic acid;
(2S)-cyclohexyl({[5-[4-(methyloxy)phenyl]-2-({[(2,4,6-trimethylphenyl)amino]carbonyl}amino)-3-thienyl]carbonyl}amino)ethanoic acid;
(2S)-cyclohexyl[({2-{[({2,6-dichloro-4-[(trifluoromethyl)oxy]phenyl}amino)carbonyl]amino}-5-[4-(methyloxy)phenyl]-3-thienyl}carbonyl)amino]ethanoic acid;
(2S)-cyclohexyl{[(2-{[({2,6-dichloro-4-[(trifluoromethyl)oxy]phenyl}amino)carbonyl]amino}-5-{4-[(trifluoromethyl)oxy]phenyl}-3-thienyl)carbonyl]amino}ethanoic acid;
(2S)-cyclohexyl[({3-{[({2,6-dichloro-4-[(trifluoromethyl)oxy]phenyl}amino)carbonyl]amino}-5-[4-(methyloxy)phenyl]-2-thienyl}carbonyl)amino]ethanoic acid;
(2S)-cyclohexyl({[5-[4-(methyloxy)phenyl]-3-({[(2,4,6-trimethylphenyl)amino]carbonyl}amino)-2-thienyl]carbonyl}amino)ethanoic acid;
N-{[5-[4-(methyloxy)phenyl]-3-({[(2,4,6-trimethylphenyl)amino]carbonyl}amino)-2-thienyl]carbonyl}-L-valine;
N-{[5-[4-(methyloxy)phenyl]-3-({[(2,4,6-trimethylphenyl)amino]carbonyl}amino)-2-thienyl]carbonyl}-L-isoleucine;
N-{[5-[4-(methyloxy)phenyl]-3-({[(2,4,6-trimethylphenyl)amino]carbonyl}amino)-2-thienyl]carbonyl}-L-norleucine;
O-(1,1-dimethylethyl)-N-{[5-[4-(methyloxy)phenyl]-3-({[(2,4,6-trimethylphenyl)amino]carbonyl}amino)-2-thienyl]carbonyl}-L-serine;
O-(1,1-dimethylethyl)-N-{[5-[4-(methyloxy)phenyl]-3-({[(2,4,6-trimethylphenyl)amino]carbonyl}amino)-2-thienyl]carbonyl}-L-threonine;
1-{[5-[4-(methyloxy)phenyl]-3-({[(2,4,6-trimethylphenyl)amino]carbonyl}amino)-2-thienyl]carbonyl}-L-proline;
1-({[5-[4-(methyloxy)phenyl]-3-({[(2,4,6-trimethylphenyl)amino]carbonyl}amino)-2-thienyl]carbonyl}amino)cyclopentanecarboxylic acid;
1-({[5-[4-(methyloxy)phenyl]-3-({[(2,4,6-trimethylphenyl)amino]carbonyl}amino)-2-thienyl]carbonyl}amino)cyclohexanecarboxylic acid;
1-({[5-[4-(methyloxy)phenyl]-3-({[(2,4,6-trimethylphenyl)amino]carbonyl}amino)-2-thienyl]carbonyl}amino)cycloheptanecarboxylic acid;
1-({[5-[4-(methyloxy)phenyl]-3-({[(2,4,6-trimethylphenyl)amino]carbonyl}amino)-2-thienyl]carbonyl}amino)cyclooctanecarboxylic acid;
(2S)-cyclohexyl({[3-({[(2,6-dichloro-4-fluorophenyl)amino]carbonyl}amino)-2-naphthalenyl]carbonyl}amino)ethanoic acid;
(2S)-cyclohexyl{[(3-{[(2,4,6-trimethylphenyl)acetyl]amino}-2-naphthalenyl)carbonyl]amino}ethanoic acid;
(2S)-cyclohexyl({[3-({[(4-ethyl-2,6-dimethylphenyl)amino]carbonyl}amino)-2-naphthalenyl]carbonyl}amino)ethanoic acid;
(2S)-cyclohexyl{[(3-{[({2,6-dichloro-4-[(trifluoromethyl)oxy]phenyl}amino)carbonyl]amino}-2-naphthalenyl)carbonyl]amino}ethanoic acid;
(2S)-(trans-4-methylcyclohexyl)({[3-({[(2,4,6-trichlorophenyl)amino]carbonyl}amino)-2-naphthalenyl]carbonyl}amino)ethanoic acid;
2-cyclohexyl-N-{[3-({[(2,4,6-trimethylphenyl)amino]carbonyl}amino)-2-naphthalenyl]carbonyl}-L-alanine;
2-cyclohexyl-N-[(3-{[({2,6-dichloro-4-[(trifluoromethyl)oxy]phenyl}amino)carbonyl]amino}-2-naphthalenyl)carbonyl]-L-alanine;
{[(3-{[({2,6-dichloro-4-[(trifluoromethyl)oxy]phenyl}amino)carbonyl]amino}-2-naphthalenyl)carbonyl]amino}[trans-4-(trifluoromethyl)cyclohexyl]acetic acid;
{[(3-{[({2,6-dichloro-4-[(trifluoromethyl)oxy]phenyl}amino)carbonyl]amino}-2-naphthalenyl)carbonyl]amino}[cis-4-(trifluoromethyl)cyclohexyl]acetic acid;
{[(3-{[({2,6-dichloro-4-[(trifluoromethyl)oxy]phenyl}amino)carbonyl]amino}-2-naphthalenyl)carbonyl]amino}(tetrahydro-2H-pyran-4-yl)acetic acid;
tetrahydro-2H-pyran-4-yl({[3-({[(2,4,6-trimethylphenyl)amino]carbonyl}amino)-2-naphthalenyl]carbonyl}amino)acetic acid;
(2S)-cyclohexyl({[3-({[(2,6-dimethyl-4-propylphenyl)amino]carbonyl}amino)-2-naphthalenyl]carbonyl}amino)ethanoic acid;
(2S)-cyclohexyl[({3-[({[2,6-dimethyl-4-(2-propyn-1-yl)phenyl]amino}carbonyl)amino]-2-naphthalenyl}carbonyl)amino]ethanoic acid;
(2S)-cyclohexyl[({3-[({[2,6-dimethyl-4-(propyloxy)phenyl]amino}carbonyl)amino]-2-naphthalenyl}carbonyl)amino]ethanoic acid;
(2S)-cyclohexyl({[2-({[(4-ethyl-2,6-dimethylphenyl)amino]carbonyl}amino)-4-fluorophenyl]carbonyl}amino)ethanoic acid;
(2S)-cyclohexyl[({2-[({[2,6-dimethyl-4-(2-propen-1-yl)phenyl]amino}carbonyl)amino]-4-fluorophenyl}carbonyl)amino]ethanoic acid;
(2S)-cyclohexyl({[2-({[(2,6-dimethyl-4-propylphenyl)amino]carbonyl}amino)-4-fluorophenyl]carbonyl}amino)ethanoic acid;
(2S)-cyclohexyl({[2-({[(2,6-dimethyl-4-pentylphenyl)amino]carbonyl}amino)-4-fluorophenyl]carbonyl}amino)ethanoic acid;
2-cyclohexyl-N-{[2-({[(2,6-dimethyl-4-propylphenyl)amino]carbonyl}amino)-4-fluorophenyl]carbonyl}-L-alanine;
(2S)-({[2-({[(4-butyl-2,6-dimethylphenyl)amino]carbonyl}amino)-4-fluorophenyl]carbonyl}amino)(cyclohexyl)ethanoic acid;
O-(1,1-dimethylethyl)-N-{[3-({[(2,6-dimethyl-4-propylphenyl)amino]carbonyl}amino)-3′,4′-difluoro-4-biphenylyl]carbonyl}-L-threonine;
(2S)-cyclohexyl[({2-[({[4-(cyclopropylmethyl)-2,6-dimethylphenyl]amino}carbonyl)amino]-4-fluorophenyl}carbonyl)amino]ethanoic acid;
N-({3-[({[4-(cyclopropylmethyl)-2,6-dimethylphenyl]amino}carbonyl)amino]-3′,4′-difluoro-4-biphenylyl}carbonyl)-O-(1,1-dimethylethyl)-L-threonine;
1-({[2-[4-(Methyloxy)phenyl]-5-({[(2,4,6-trimethylphenyl)amino]carbonyl}amino)-1,3-thiazol-4-yl]carbonyl}amino)cyclohexanecarboxylic acid;
(2S)-(4-hydroxyphenyl)({[3-({[(2,4,6-trimethylphenyl)amino]carbonyl}amino)-2-naphthalenyl]carbonyl}amino)ethanoic acid;
(2S)-(4-hydroxycyclohexyl)({[3-({[(2,4,6-trimethylphenyl)amino]carbonyl}amino)-2-naphthalenyl]carbonyl}amino)ethanoic acid;
N⁴,N⁴-dimethyl-N²-{[4′-(methyloxy)-3-({[(2,4,6-trimethylphenyl)amino]carbonyl}amino)-4-biphenylyl]carbonyl}-L-asparagine;
N-({3-[({[4-(cyclopropylmethyl)-2,6-dimethylphenyl]amino}carbonyl)amino]-3′-fluoro-4-biphenylyl}carbonyl)-O-(1,1-dimethylethyl)-L-threonine;
N-{[3′-fluoro-3-({[(2,4,6-trimethylphenyl)amino]carbonyl}amino)-4-biphenylyl]carbonyl}-L-aspartic acid;
O-(Phenylmethyl)-N-{[3-({[(2,4,6-trimethylphenyl)amino]carbonyl}amino)-2-naphthalenyl]carbonyl}-L-serine;
N-{[3′,4′-Difluoro-3-({[(2,4,6-trimethylphenyl)amino]carbonyl}amino)-4-biphenylyl]carbonyl}-O-(phenylmethyl)-L-serine;
(3R)-5-Methyl-3-[(phenylmethyl)oxy]-N-{[3-({[(2,4,6-trimethylphenyl)amino)carbonyl}amino)-2-naphthalenyl]carbonyl}-L-norleucine;
O-cyclobutyl-N-{[3-({[(2,4,6-trimethylphenyl)amino]carbonyl}amino)-2-naphthalenyl]carbonyl}-L-threonine;
N-{[3-({[(2,4,6-trimethylphenyl)amino]carbonyl}amino)-2-naphthalenyl]carbonyl}-L-phenylalanine;
(2S)-4-({[(1,1-dimethylethyl)oxy]carbonyl}amino)-2-({[3-({[(2,4,6-trimethylphenyl)amino]carbonyl}amino)-2-naphthalenyl]carbonyl}amino)butanoic acid;
5,5-dimethyl-N-{[3-({[(2,4,6-trimethylphenyl)amino]carbonyl}amino)-2-naphthalenyl]carbonyl}norleucine;
O-cyclobutyl-N-{[3′,4′-difluoro-3-({[(2,4,6-trimethylphenyl)amino]carbonyl}amino)-4-biphenylyl]carbonyl}-L-threonine;
O-(1-methylcyclopentyl)-N-{[3-({[(2,4,6-trimethylphenyl)amino]carbonyl}amino)-2-naphthalenyl]carbonyl}-L-threonine;
(2S)-cyclohexyl({[2′-(methyloxy)-3-({[(2,4,6-trimethylphenyl)amino]carbonyl}amino)-4-biphenylyl]carbonyl}amino)ethanoic acid;
O-(1,1-Dimethylethyl)-N-{[2′-(methyloxy)-3-({[(2,4,6-trimethylphenyl)amino]carbonyl}amino)-4-biphenylyl]carbonyl}-L-threonine;
N-{[3′,5′-Difluoro-3-({[(2,4,6-trimethylphenyl)amino]carbonyl}amino)-4-biphenylyl]carbonyl}-O-(1,1-dimethylethyl)-L-threonine;
(2S)-Cyclohexyl({[3′,5′-difluoro-3-({[(2,4,6-trimethylphenyl)amino]carbonyl}amino)-2-biphenylyl]carbonyl}amino)ethanoic acid;
O-(1,1-Dimethylethyl)-N-{[4′-fluoro-3-{[({2,4,6-trimethylphenyl)amino]carbonyl}amino)-4-biphenylyl]carbonyl}-L-threonine;
O-(1,1-Dimethylethyl)-N-{[3-({[(2,4,6-trimethylphenyl)amino]carbonyl}amino)-4-biphenylyncarbonyl}-L-threonine;
1-({[3-({[(2,4,6-Trimethylphenyl)amino]carbonyl}amino)-4-biphenylyl]carbonyl}amino)cyclooctanecarboxylic acid;
N-{[3-({[(4-Cyclopropyl-2,6-dimethylphenyl)amino)carbonyl}amino)-3′-fluoro-4-biphenylyl]carbonyl}-O-(1,1-dimethylethyl)-L-threonine;
(2S)-cyclohexyl({[3-({[(4-cyclopropylphenyl)amino]carbonyl}amino)-2-naphthalenyl]carbonyl}amino)ethanoic acid;
N-{[3-({[(4-cyclopropyl-2,6-dimethylphenyl)amino]carbonyl}amino)-4′-(methyloxy)-4-biphenylyl]carbonyl}-O-(1,1-dimethylethyl)-L-threonine;
1-({[5-(4-chlorophenyl)-3-({[(2,4,6-trimethylphenyl)amino]carbonyl}amino)-2-thienyl]carbonyl}amino)cyclohexanecarboxylic acid; and
1-({[5-(3,4-difluorophenyl)-3-({[(2,4,6-trimethylphenyl)amino]carbonyl}amino)-2-thienyl]carbonyl}amino)cyclohexanecarboxylic acid.

36. A pharmaceutical composition comprising a compound of claim 1, a pharmaceutically acceptable salt, solvate, or physiologically functional derivative thereof and at least one excipient.

37. A method of treating a mammal suffering from diabetes, a condition associated with diabetes, or both comprising the administration of a compound of claim 1, a pharmaceutically acceptable salt, solvate, or physiologically functional derivative thereof.

38. The method of claim 37 wherein said mammal is a human.

39. A method of treating a mammal suffering from diabetes, a condition associated with diabetes, or both comprising the administration to said mammal of a pharmaceutical composition comprising a compound of claim 1, a pharmaceutically acceptable salt, solvate, or physiologically functional derivative thereof and at least one excipient.

40. The method of claim 39 wherein said mammal is a human.

41. A method of treating a mammal suffering from tissue ischemia, myocardial ischemia, or both comprising the administration of a compound of claim 1, a pharmaceutically acceptable salt, solvate, or physiologically functional derivative thereof.

42. The method of claim 41 wherein said mammal is a human.

43. A method of treating a mammal suffering from tissue ischemia, myocardial ischemia, or both comprising the administration to said mammal of a pharmaceutical composition of a compound of claim 1, a pharmaceutically acceptable salt, solvate, or physiologically functional derivative thereof and at least one excipient.

44. The method of claim 43 wherein said mammal is a human.

45. A process of making a compound of claim 1 comprising a solid-phase synthesis using at least one isocyanate.

46. A process of making a compound of claim 1 comprising a solid-phase synthesis using at least one urea carboxylic acid.

47. A process of making a compound of claim 1 comprising a solution-phase synthesis using at least one urea carboxylic acid.

48. A process of making a compound of claim 1 comprising a solid-phase synthesis using at least one acid chloride.

49. A process of making a compound of claim 1 comprising a solution-phase synthesis using at least one isocyanate.

50. A process of making a compound of claim 1 comprising a solution-phase synthesis using at least one carboxylic acid.

51.-54. (canceled)
